(19) United States
(12) Patent Application Publication
(10) Pub. No.: US 2025/0297008 A1
(43) Pub. Date: Sep. 25, 2025

(54) MULTIFUNCTIONAL MOLECULES BINDING TO TCR AND USES THEREOF

(71) Applicant: Marengo Therapeutics, Inc., Cambridge, MA (US)

(72) Inventor: Andrew Bayliffe, London (GB)

(21) Appl. No.: 19/175,671

(22) Filed: Apr. 10, 2025

Related U.S. Application Data
(63) Continuation of application No. PCT/US23/34966, filed on Oct. 11, 2023.
(60) Provisional application No. 63/379,271, filed on Oct. 12, 2022.
(60) Provisional application No. 63/381,231, filed on Oct. 27, 2022.

(51) Int. Cl. C07K 16/28; A61K 47/02; A61P 35/00; C07K 14/55

(52) U.S. Cl. CPC C07K 16/2809; A61K 47/02; A61P 35/00; C07K 14/55; C07K 2319/00

(57) ABSTRACT

Provides herein are multifunctional polypeptide molecules comprising T cell receptor variable beta-binding moieties and cytokines and methods of treating conditions or diseases in a subject using the same.

### CROSS REFERENCE

This application is a continuation of International Application No. PCT/US2023/034966 filed Oct. 11, 2023, which claims the benefits of U.S. Provisional Application No. 63/379,271 filed Oct. 12, 2022, and U.S. Provisional Application No. 63/381,231 filed Oct. 27, 2022, each of which is incorporated herein by reference in its entirety.

### SEQUENCE LISTING

The instant application contains a Sequence listing which has been submitted electronically in XML format and is herein incorporated by reference in its entirety. Said XML copy, created on Jan. 9, 2024, is named 53676-757_601_SL.xml and is 1,187,512 bytes in size.

### BACKGROUND

Currently available molecules designed to redirect T cells to promote tumor cell lysis for cancer immunotherapy typically target the CD3 epsilon (CD3e) subunit of the T cell receptor (TCR). However, there are limitations to this approach. Previous studies have shown that, e.g., low doses of anti-CD3e monoclonal antibody (mAb) can cause T cell dysfunction and exert immunosuppressive effects. In addition, anti-CD3e mAbs bind to all T cells and thus activate a large number of T cells. Such non-physiological massive activation of T cells by these anti-CD3e mAbs can result in the production of proinflammatory cytokines such as IFN-gamma, IL-1-beta, IL-6, IL-10 and TNF-alpha, causing a “cytokine storm” known as the cytokine release syndrome (CRS), which is also associated with neurotoxicity (NT). Thus, there is a need for improved T cell receptor-binding molecules that redirect T cells for cancer immunotherapy.

### SUMMARY

Provided herein is a method of treating cancer in a human subject in need thereof comprising administering to the human subject a multifunctional molecule, wherein the multifunctional molecule comprises a TCRβV6-binding moiety, and an interleukin-2 (IL-2) or a functional fragment or a functional variant thereof, wherein the multifunctional molecule is administered to the human subject at a first dose of from about 0.001 mg/kg to about 10 mg/kg; thereby treating the cancer in the human subject.

Also provided herein is a method of treating cancer in a human subject in need thereof comprising administering to the human subject a multifunctional molecule, wherein the multifunctional molecule comprises a TCRβV6-binding moiety, and an interleukin-2 (IL-2) or a functional fragment or a functional variant thereof, wherein administering comprises administering multiple doses of the multifunctional molecule to the human subject.

Also provided herein is a method of treating cancer in a human subject in need thereof comprising administering to the human subject a first dose of a multifunctional molecule, wherein the multifunctional molecule comprises a TCRβV6-binding moiety, and an interleukin-2 (IL-2) or a functional fragment or a functional variant thereof, wherein the human subject is characterized as having a solid tumor, and wherein if the human subject had a symptomatic central nervous system (CNS) metastases, the human subject has previously been treated for the symptomatic central nervous system (CNS) metastases, has been asymptomatic for 14 days or more and is not currently receiving treatment for CNS disease, and does not currently have leptomeningeal disease or cord compression; and if the human subject had previously been treated with a checkpoint inhibitor therapy (CPI), the human subject has CPI immune-related toxicity resolved to either Grade ≤1 or baseline relative to before being treated with the CPI.

Also provided herein is a method of treating cancer in a human subject in need thereof comprising administering to the human subject a first dose of a multifunctional molecule, wherein the multifunctional molecule comprises a TCRβV6-binding moiety, and an interleukin-2 (IL-2) or a functional fragment or a functional variant thereof, wherein the human subject: does not have a history of autoimmune disease; does not have a major surgery or traumatic injury within 8 weeks before a first administration of the multifunctional molecule or the subject does not have an unhealed wound from surgery or injury; is not treated with >10 mg per day of an immune-suppressive drug within 7 days prior to a first administration of the multifunctional molecule; is not previously treated with a cytotoxic chemotherapy, a small molecule inhibitor, radiation, or an interventional radiology procedure with 2 weeks prior to a first administration of the multifunctional molecule; is not previously treated with a monoclonal antibody, an antibody-drug conjugate, a radioimmunoconjugate within 6 weeks prior to a first administration of the multifunctional molecule; does not have an inflammatory process that is not resolved within 4 weeks before a first administration of the multifunctional molecule; does not have a clinically significant pulmonary compromise; or does not have an active viral, bacterial, or systemic fungal infection requiring parenteral treatment within 7 days of a first administration of the multifunctional molecule.

In some embodiments, the first dose is the first of multiple doses. In some embodiments, the human subject is characterized as having a solid tumor.

In some embodiments, if the human subject had a symptomatic central nervous system (CNS) metastases, the human subject has previously been treated for the symptomatic central nervous system (CNS) metastases, has been asymptomatic for 14 days or more and is not currently receiving treatment for CNS disease, and does not currently have leptomeningeal disease or cord compression; and if the human subject had previously been treated with a checkpoint inhibitor therapy (CPI), the human subject has CPI immune-related toxicity resolved to either Grade ≤1 or baseline relative to before being treated with the CPI.

In some embodiments, the solid tumor is selected from the group consisting of high mutational burden (TMB-H), microsatellite instability/DNA mismatch repair (MSI-H/dMMR), virally associated tumor, metastatic triple-negative breast cancer (mTNBC), relapsed and refractory epithelial ovarian cancer, metastatic castration-resistant prostate cancer (mCRPC); K-Ras wild type CRC; K-Ras mutant CRC and primary stage IV or recurrent non-small cell lung cancer (NSCLC).

In some embodiments, the virally associated tumor comprises Merkel cell carcinoma, cervical cancer, oropharyngeal cancer, anal cancer, penile cancer, vaginal cancer, or vulvar cancer.

In some embodiments, the human subject is not concurrently accepting treatment for a CNS disease, the subject does not have leptomeningeal disease, or the subject does not have cord compression.

In some embodiments, a CPI immune-related toxicity of the subject is Grade ≤1 or baseline, the subject has experienced CPI-related endocrine abnormalities, or the subject has not experienced CPI-related Grade 3-4 pneumonitis, peri/myocarditis, colitis and bowel perforation, myositis, encephalitis, or peripheral neuropathy.

In some embodiments, the human subject: does not have a history of autoimmune disease other than: vitiligo; psoriasis, atopic dermatitis or other autoimmune skin condition not requiring systemic treatment; Graves' disease, now euthyroid for >4 weeks; hypothyroidism managed by thyroid replacement; Alopecia; Arthritis managed without systemic therapy beyond oral nonsteroidal anti-inflammatory drugs, and Adrenal insufficiency well controlled on replacement therapy; does not have a major surgery or traumatic injury within 8 weeks before a first administration of the multifunctional molecule or the subject does not have an unhealed wound from surgery or injury; is not treated with >10 mg per day of an immune-suppressive drug within 7 days prior to a first administration of the multifunctional molecule: is not previously treated with a cytotoxic chemotherapy, a small molecule inhibitor, radiation, or an interventional radiology procedure with 2 weeks prior to a first administration of the multifunctional molecule; is not previously treated with a monoclonal antibody, an antibody-drug conjugate, a radioimmunoconjugate within 6 weeks prior to a first administration of the multifunctional molecule; does not have an inflammatory process that is not resolved within 4 weeks before a first administration of the multifunctional molecule; does not have a clinically significant pulmonary compromise; or does not have an active viral, bacterial, or systemic fungal infection requiring parenteral treatment within 7 days of a first administration of the multifunctional molecule.

In some embodiments, the autoimmune disease does not comprise vitiligo; psoriasis, atopic dermatitis or other autoimmune skin condition not requiring systemic treatment; Graves' disease, now euthyroid for >4 weeks; hypothyroidism managed by thyroid replacement: Alopecia; Arthritis managed without systemic therapy beyond oral nonsteroidal anti-inflammatory drugs, and Adrenal insufficiency well controlled on replacement therapy.

In some embodiments, the human subject is at least 18 years old.

In some embodiments, the multifunctional molecule is administered to the human subject at a first dose of from about 0.001 mg/kg to about 10 mg/kg. In some embodiments, the multifunctional molecule is administered at the first dose of about 0.001 mg/kg to about 1 mg/kg. In some embodiments, the multifunctional molecule is administered at the first dose of about 0.001 mg/kg to about 5 mg/kg. In some embodiments, the multifunctional molecule is administered at the first dose of about 0.001 mg/kg to about 10 mg/kg. In some embodiments, the multifunctional molecule is administered at the first dose of about 0.005 mg/kg to about 1 mg/kg. In some embodiments, the multifunctional molecule is administered at the first dose of about 0.005 mg/kg to about 5 mg/kg. In some embodiments, the multifunctional molecule is administered at the first dose of about 0.005 mg/kg to about 10 mg/kg. In some embodiments, the multifunctional molecule is administered at the first dose of about 0.01 mg/kg to about 1 mg/kg. In some embodiments, the multifunctional molecule is administered at the first dose of about 0.01 mg/kg to about 5 mg/kg. In some embodiments, the multifunctional molecule is administered at the first dose of about 0.01 mg/kg to about 10 mg/kg. In some embodiments, the multifunctional molecule is administered at the first dose of about 0.05 mg/kg to about 1 mg/kg. In some embodiments, the multifunctional molecule is administered at the first dose of about 0.05 mg/kg to about 5 mg/kg. In some embodiments, the multifunctional molecule is administered at the first dose of about 0.05 mg/kg to about 10 mg/kg. In some embodiments, the multifunctional molecule is administered at the first dose of about 0.1 mg/kg to about 1 mg/kg. In some embodiments, the multifunctional molecule is administered at the first dose of about 0.1 mg/kg to about 5 mg/kg. In some embodiments, the multifunctional molecule is administered at the first dose of about 0.1 mg/kg to about 10 mg/kg.

In some embodiments, the multifunctional molecule is administered at the first dose of 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.11 mg/kg, 0.12 mg/kg, 0.13 mg/kg, 0.14 mg/kg, 0.15 mg/kg, 0.16 mg/kg, 0.17 mg/kg, 0.18 mg/kg, 0.19 mg/kg, 0.2 mg/kg, 0.21 mg/kg, 0.22 mg/kg, 0.23 mg/kg, 0.24 mg/kg, 0.25 mg/kg, 0.26 mg/kg, 0.27 mg/kg, 0.28 mg/kg, 0.29 mg/kg, 0.3 mg/kg, 0.31 mg/kg, 0.32 mg/kg, 0.33 mg/kg, 0.34 mg/kg, 0.35 mg/kg, 0.36 mg/kg, 0.37 mg/kg, 0.38 mg/kg, 0.39 mg/kg, 0.4 mg/kg, 0.41 mg/kg, 0.42 mg/kg, 0.43 mg/kg, 0.44 mg/kg, 0.45 mg/kg, 0.46 mg/kg, 0.47 mg/kg, 0.48 mg/kg, 0.49 mg/kg, 0.5 mg/kg, 0.51 mg/kg, 0.52 mg/kg, 0.53 mg/kg, 0.54 mg/kg, 0.55 mg/kg, 0.56 mg/kg, 0.57 mg/kg, 0.58 mg/kg, 0.59 mg/kg, 0.6 mg/kg, 0.61 mg/kg, 0.62 mg/kg, 0.63 mg/kg, 0.64 mg/kg, 0.65 mg/kg, 0.66 mg/kg, 0.67 mg/kg, 0.68 mg/kg, 0.69 mg/kg, 0.7 mg/kg, 0.71 mg/kg, 0.72 mg/kg, 0.73 mg/kg, 0.74 mg/kg, 0.75 mg/kg, 0.76 mg/kg, 0.77 mg/kg, 0.78 mg/kg, 0.79 mg/kg, 0.8 mg/kg, 0.81 mg/kg, 0.82 mg/kg, 0.83 mg/kg, 0.84 mg/kg, 0.85 mg/kg, 0.86 mg/kg, 0.87 mg/kg, 0.88 mg/kg, 0.89 mg/kg, 0.9 mg/kg, 0.91 mg/kg, 0.92 mg/kg, 0.93 mg/kg, 0.94 mg/kg, 0.95 mg/kg, 0.96 mg/kg, 0.97 mg/kg, 0.98 mg/kg, 0.99 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 16.5 mg/kg, 17 mg/kg, 17.5 mg/kg, 18 mg/kg, 18.5 mg/kg, 19 mg/kg, 19.5 mg/kg, 20 mg/kg, 20.5 mg/kg, 21 mg/kg, 21.5 mg/kg, 22 mg/kg, 22.5 mg/kg, 23 mg/kg, 23.5 mg/kg, 24 mg/kg, 24.5 mg/kg, 25 mg/kg, 25.5 mg/kg, 26 mg/kg, 26.5 mg/kg, 27 mg/kg, 27.5 mg/kg, 28 mg/kg, 28.5 mg/kg, 29 mg/kg, 29.5 mg/kg, 30 mg/kg, 30.5 mg/kg, 31 mg/kg, 31.5 mg/kg, 32 mg/kg, 32.5 mg/kg, 33 mg/kg, 33.5 mg/kg, 34 mg/kg, 34.5 mg/kg, 35 mg/kg, 35.5 mg/kg, 36 mg/kg, 36.5 mg/kg, 37 mg/kg, 37.5 mg/kg, 38 mg/kg, 38.5 mg/kg, 39 mg/kg, 39.5 mg/kg, 40 mg/kg, 40.5 mg/kg, 41 mg/kg, 41.5 mg/kg, 42 mg/kg, 42.5 mg/kg, 43 mg/kg, 43.5 mg/kg, 44 mg/kg, 44.5 mg/kg, 45 mg/kg, 45.5 mg/kg, 46 mg/kg, 46.5 mg/kg, 47 mg/kg, 47.5 mg/kg, 48 mg/kg, 48.5 mg/kg, 49 mg/kg, 49.5 mg/kg, or 50 mg/kg.

In some embodiments, the administering comprises administering multiple doses of the multifunctional molecule to the human subject.

In some embodiments, a subsequent dose of the multiple doses is lower than a previous dose immediately preceding the subsequent dose following an indication that administration of the previous dose is not tolerated. In some embodiments, a subsequent dose of the multiple doses is the same as a previous dose immediately preceding the subsequent dose following an indication that administration of the previous dose is tolerated. In some embodiments, a subsequent dose of the multiple doses is higher than a previous dose immediately preceding the subsequent dose following an indication that administration of the previous dose is tolerated.

In some embodiments, a subsequent dose of the multiple doses is the same as a previous dose immediately preceding the subsequent dose following an indication that administration of the previous dose is effective. In some embodiments, a subsequent dose of the multiple doses is lower than a previous dose immediately preceding the subsequent dose following an indication that administration of the previous dose is effective. In some embodiments, a subsequent dose of the multiple doses is higher than a previous dose immediately preceding the subsequent dose following an indication that administration of the previous dose is not effective.

In some embodiments, a subsequent dose of the multiple doses is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after administration of a previous dose immediately preceding the subsequent dose. In some embodiments, a subsequent dose of the multiple doses is administered at least 1, 2, 3, or 4 weeks after administration of a previous dose immediately preceding the subsequent dose. In some embodiments, a subsequent dose of the multiple doses is administered at least 1, 2, 3, 4, 5, 10, 11 or 12 months after administration of a previous dose immediately preceding the subsequent dose.

In some embodiments, dose frequency of the multiple doses is maintained or reduced following an indication that a previous dose immediately preceding the subsequent dose is effective. In some embodiments, dose frequency of the administering is increased following an indication that a dose of the multiple doses is not effective. In some embodiments, the method comprises administering the multifunctional molecule to the human subject once every week.

In some embodiments, the method comprises administering the multifunctional molecule to the human subject once every week for at least 1, 2, 3, or 4 weeks, or at least 1, 2, 3, 4, 5, 10, 11 or 12 months, or at least 1, 2, or 3 years. In some embodiments, the method comprises administering the multifunctional molecule to the human subject once every two weeks. In some embodiments, the method comprises administering the multifunctional molecule to the human subject once every two weeks for at least 1, 2, 3, or 4 weeks, or at least 1, 2, 3, 4, 5, 10, 11 or 12 months, or at least 1, 2, or 3 years. In some embodiments, the method comprises administering the multifunctional molecule to the human subject once every three weeks. In some embodiments, the method comprises administering the multifunctional molecule to the human subject once every three weeks for at least 1, 2, 3, or 4 weeks, or at least 1, 2, 3, 4, 5, 10, 11 or 12 months, or at least 1, 2, or 3 years. In some embodiments, the method comprises administering the multifunctional molecule to the human subject once every two weeks for 28 days within which the multifunctional molecule is administered to the human subject on day 1 and day 15.

In some embodiments, the multifunctional molecule is administered by intravenous infusion. In some embodiments, the multifunctional molecule is administered subcutaneously, intratumorally, intranodally, intramuscularly, intradermally, or intraperitoneally.

In some embodiments, the multifunctional molecule is administered by intravenous infusion over a time course of from about 25 minutes to about 240 minutes. In some embodiments, the multifunctional molecule is administered by intravenous infusion over a time course of from about 105 minutes to 120 minutes or from about 125 minutes to 145 minutes. In some embodiments, the multifunctional molecule is administered by intravenous infusion over a time course of from about 150 minutes to 200 minutes or from about 160 minutes to 190 minutes. In some embodiments, the multifunctional molecule is administered by intravenous infusion over a time course of from about 25 minutes to about 35 minutes or from about 55 minutes to about 65 minutes. In some embodiments, the multifunctional molecule is administered by intravenous infusion over a time course of from about 35 minutes to about 50 minutes or from about 85 minutes to about 95 minutes.

In some embodiments, the method further comprises administrating at least one additional therapeutic agent or therapy. In some embodiments, the at least one additional therapeutic agent or therapy is administered at the same time as a dose of the multifunctional molecule. In some embodiments, the at least one additional therapeutic agent or therapy is administered prior to administration a dose of the multifunctional molecule. In some embodiments, the at least one additional therapeutic agent or therapy is administered after administration of a dose of the multifunctional molecule.

In some embodiments, administering comprises administering a pharmaceutical composition comprising the multifunctional molecule, and wherein the pharmaceutical composition further comprises a pharmaceutically acceptable excipient, carrier or diluent. In some embodiments, the pharmaceutical composition is a liquid composition. In some embodiments, the pharmaceutical composition comprises a pharmaceutically acceptable diluent that is a saline solution. In some embodiments, the pharmaceutical composition comprises a pharmaceutically acceptable diluent that is a 0.9% saline solution.

In some embodiments, the multifunctional molecule is present in the pharmaceutical composition at a concentration of from about 0.02 mg/mL to about 15 mg/mL. In some embodiments, the multifunctional molecule is present in the pharmaceutical composition at a concentration of from about 0.2 mg/mL to about 15 mg/mL. In some embodiments, the multifunctional molecule is present in the pharmaceutical composition at a concentration of from about 0.02 mg/mL to about 1.5 mg/mL. In some embodiments, the multifunctional molecule is present in the pharmaceutical composition at a concentration of from about 0.2 mg/mL to about 1.5 mg/mL.

In some embodiments, the multifunctional molecule is present in the pharmaceutical composition at a concentration of about 0.02 mg/mL, 0.03 mg/mL, 0.04 mg/mL, 0.05 mg/mL, 0.06 mg/mL, 0.07 mg/mL, 0.08 mg/mL, 0.09 mg/mL, 0.1 mg/mL, 0.11 mg/mL, 0.12 mg/mL, 0.13 mg/mL, 0.14 mg/mL, 0.15 mg/mL, 0.16 mg/mL, 0.17 mg/mL, 0.18 mg/mL, 0.19 mg/mL, 0.2 mg/mL, 0.25 mg/mL, 0.3 mg/mL, 0.35 mg/mL, 0.4 mg/mL, 0.45 mg/mL, 0.5 mg/mL, 0.55 mg/mL, 0.6 mg/mL, 0.65 mg/mL, 0.7 mg/mL, 0.75 mg/mL, 0.8 mg/mL, 0.85 mg/mL, 0.9 mg/mL, 0.95 mg/mL, 1 mg/mL, 1.05 mg/mL, 1.1 mg/mL, 1.15 mg/mL, 1.2 mg/mL, 1.25 mg/mL, 1.3 mg/mL, 1.35 mg/mL, 1.4 mg/mL, 1.45 mg/mL, 1.5 mg/mL, 1.55 mg/mL, 1.6 mg/mL, 1.65 mg/mL, 1.7 mg/mL, 1.75 mg/mL, 1.8 mg/mL, 1.85 mg/mL, 1.9 mg/mL, 1.95 mg/mL, 2 mg/mL, 2.05 mg/mL, 2.1 mg/mL, 2.15 mg/mL, 2.2 mg/mL, 2.25 mg/mL, 2.3 mg/mL, 2.35 mg/mL, 2.4 mg/mL, 2.45 mg/mL, 2.5 mg/mL, 3 mg/mL, 3.5 mg/mL, 4 mg/mL, 4.5 mg/mL, 5 mg/mL, 5.5 mg/mL, 6 mg/mL, 6.5 mg/mL, 7 mg/mL, 7.5 mg/mL, 8 mg/mL, 8.5 mg/mL, 9 mg/mL, 9.5 mg/mL, 10 mg/mL, 10.5 mg/mL, 11 mg/mL, 11.5 mg/mL, 12 mg/mL, 12.5 mg/mL, 13 mg/mL, 13.5 mg/mL, 14 mg/mL, 14.5 mg/mL, or 15 mg/mL.

In some embodiments, the pharmaceutical composition comprises from about 0.5 mL to about 500 mL of a diluent.

Also provided herein is a dose of a pharmaceutical composition comprising a multifunctional molecule, wherein the multifunctional molecule comprises a TCRβV6-binding moiety, and an interleukin-2 (IL-2) or a functional fragment or a functional variant thereof, wherein the dose is from about 0.001 mg/kg to about 10 mg/kg of the multifunctional molecule.

In some embodiments, the dose is from about 0.001 mg/kg to about 1 mg/kg of the multifunctional molecule. In some embodiments, the dose is from about 0.001 mg/kg to about 5 mg/kg of the multifunctional molecule. In some embodiments, the dose is from about 0.001 mg/kg to about 10 mg/kg of the multifunctional molecule. In some embodiments, the dose is from about 0.005 mg/kg to about 1 mg/kg of the multifunctional molecule. In some embodiments, the multifunctional molecule is administered at a first dose of about 0.005 mg/kg to about 5 mg/kg of the multifunctional molecule. In some embodiments, the dose is from about 0.005 mg/kg to about 10 mg/kg of the multifunctional molecule. In some embodiments, the dose is from about 0.01 mg/kg to about 1 mg/kg of the multifunctional molecule. In some embodiments, the dose is from about 0.01 mg/kg to about 5 mg/kg of the multifunctional molecule. In some embodiments, the dose is from about 0.01 mg/kg to about 10 mg/kg of the multifunctional molecule. In some embodiments, the dose is from about 0.05 mg/kg to about 1 mg/kg of the multifunctional molecule. In some embodiments, the dose is from about 0.05 mg/kg to about 5 mg/kg of the multifunctional molecule. In some embodiments, the dose is from about 0.05 mg/kg to about 10 mg/kg of the multifunctional molecule. In some embodiments, the dose is from about 0.1 mg/kg to about 1 mg/kg of the multifunctional molecule. In some embodiments, the dose is from about 0.1 mg/kg to about 5 mg/kg of the multifunctional molecule. In some embodiments, the dose is from about 0.1 mg/kg to about 10 mg/kg of the multifunctional molecule.

In some embodiments, the dose is about 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.11 mg/kg, 0.12 mg/kg, 0.13 mg/kg, 0.14 mg/kg, 0.15 mg/kg, 0.16 mg/kg, 0.17 mg/kg, 0.18 mg/kg, 0.19 mg/kg, 0.2 mg/kg, 0.21 mg/kg, 0.22 mg/kg, 0.23 mg/kg, 0.24 mg/kg, 0.25 mg/kg, 0.26 mg/kg, 0.27 mg/kg, 0.28 mg/kg, 0.29 mg/kg, 0.3 mg/kg, 0.31 mg/kg, 0.32 mg/kg, 0.33 mg/kg, 0.34 mg/kg, 0.35 mg/kg, 0.36 mg/kg, 0.37 mg/kg, 0.38 mg/kg, 0.39 mg/kg, 0.4 mg/kg, 0.41 mg/kg, 0.42 mg/kg, 0.43 mg/kg, 0.44 mg/kg, 0.45 mg/kg, 0.46 mg/kg, 0.47 mg/kg, 0.48 mg/kg, 0.49 mg/kg, 0.5 mg/kg, 0.51 mg/kg, 0.52 mg/kg, 0.53 mg/kg, 0.54 mg/kg, 0.55 mg/kg, 0.56 mg/kg, 0.57 mg/kg, 0.58 mg/kg, 0.59 mg/kg, 0.6 mg/kg, 0.61 mg/kg, 0.62 mg/kg, 0.63 mg/kg, 0.64 mg/kg, 0.65 mg/kg, 0.66 mg/kg, 0.67 mg/kg, 0.68 mg/kg, 0.69 mg/kg, 0.7 mg/kg, 0.71 mg/kg, 0.72 mg/kg, 0.73 mg/kg, 0.74 mg/kg, 0.75 mg/kg, 0.76 mg/kg, 0.77 mg/kg, 0.78 mg/kg, 0.79 mg/kg, 0.8 mg/kg, 0.81 mg/kg, 0.82 mg/kg, 0.83 mg/kg, 0.84 mg/kg, 0.85 mg/kg, 0.86 mg/kg, 0.87 mg/kg, 0.88 mg/kg, 0.89 mg/kg, 0.9 mg/kg, 0.91 mg/kg, 0.92 mg/kg, 0.93 mg/kg, 0.94 mg/kg, 0.95 mg/kg, 0.96 mg/kg, 0.97 mg/kg, 0.98 mg/kg, 0.99 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 16.5 mg/kg, 17 mg/kg, 17.5 mg/kg, 18 mg/kg, 18.5 mg/kg, 19 mg/kg, 19.5 mg/kg, 20 mg/kg, 20.5 mg/kg, 21 mg/kg, 21.5 mg/kg, 22 mg/kg, 22.5 mg/kg, 23 mg/kg, 23.5 mg/kg, 24 mg/kg, 24.5 mg/kg, 25 mg/kg, 25.5 mg/kg, 26 mg/kg, 26.5 mg/kg, 27 mg/kg, 27.5 mg/kg, 28 mg/kg, 28.5 mg/kg, 29 mg/kg, 29.5 mg/kg, 30 mg/kg, 30.5 mg/kg, 31 mg/kg, 31.5 mg/kg, 32 mg/kg, 32.5 mg/kg, 33 mg/kg, 33.5 mg/kg, 34 mg/kg, 34.5 mg/kg, 35 mg/kg, 35.5 mg/kg, 36 mg/kg, 36.5 mg/kg, 37 mg/kg, 37.5 mg/kg, 38 mg/kg, 38.5 mg/kg, 39 mg/kg, 39.5 mg/kg, 40 mg/kg, 40.5 mg/kg, 41 mg/kg, 41.5 mg/kg, 42 mg/kg, 42.5 mg/kg, 43 mg/kg, 43.5 mg/kg, 44 mg/kg, 44.5 mg/kg, 45 mg/kg, 45.5 mg/kg, 46 mg/kg, 46.5 mg/kg, 47 mg/kg, 47.5 mg/kg, 48 mg/kg, 48.5 mg/kg, 49 mg/kg, 49.5 mg/kg, or 50 mg/kg of the multifunctional molecule.

Also provided herein is a pharmaceutical composition comprising a multifunctional molecule and a pharmaceutically acceptable diluent, wherein the multifunctional molecule comprises a TCRβV6-binding moiety, and an interleukin-2 (IL-2) or a functional fragment or a functional variant thereof, wherein the pharmaceutically acceptable diluent is a saline solution.

In some embodiments, the pharmaceutically acceptable diluent is a 0.9% saline solution.

In some embodiments, the multifunctional molecule is present in the pharmaceutical composition at a concentration of from about 0.02 mg/mL to about 15 mg/mL or from about 0.2 mg/mL to about 1.5 mg/mL. In some embodiments, the pharmaceutical composition has a total volume of from about 0.5 mL to about 500 mL. In some embodiments, the pharmaceutical composition has a total volume of from about 5 mL to about 500 mL. In some embodiments, the pharmaceutical composition has a total volume of from about 50 mL to about 500 mL. In some embodiments, the pharmaceutical composition has a total volume of from about 0.5 mL to about 350 mL. In some embodiments, the pharmaceutical composition has a total volume of from about 0.5 mL to about 250 mL. In some embodiments, the pharmaceutical composition has a total volume of from about 0.5 mL to about 150 mL. In some embodiments, the pharmaceutical composition has a total volume of from about 0.5 mL to about 50 mL.

Also provided herein is a pharmaceutical composition comprising a multifunctional molecule and a pharmaceutically acceptable diluent, wherein the multifunctional molecule comprises a TCRβV6-binding moiety, and an interleukin-2 (IL-2) or a functional fragment or a functional variant thereof, wherein the multifunctional molecule is present in the pharmaceutical composition at a concentration of from about 0.02 mg/mL to about 15 mg/mL.

Also provided herein is a pharmaceutical composition comprising a multifunctional molecule and a pharmaceutically acceptable diluent, wherein the multifunctional molecule comprises a TCRβV6-binding moiety, and an interleukin-2 (IL-2) or a functional fragment or a functional variant thereof, wherein the pharmaceutical composition comprises from about 0.1 mg to about 500 mg of the multifunctional molecule.

In some embodiments, the pharmaceutical composition comprises from about 0.5 mg to about 200 mg of the multifunctional molecule. In some embodiments, the pharmaceutical composition comprises from about 0.5 mg to about 100 mg of the multifunctional molecule. In some embodiments, the pharmaceutical composition comprises from about 1 mg to about 200 mg of the multifunctional molecule. In some embodiments, the pharmaceutical composition comprises from about 1 mg to about 100 mg of the multifunctional molecule.

Also provided herein is a pharmaceutical composition comprising a multifunctional molecule and a pharmaceutically acceptable excipient, wherein the multifunctional molecule comprises: a TCRβV6-binding moiety, and an interleukin-2 (IL-2) or a functional fragment or a functional variant thereof, wherein the pharmaceutically acceptable excipient comprises one or more of L-histidine/L-histidine monohydrochloride buffer, sucrose, or polysorbate.

In some embodiments, the pharmaceutical composition comprises from about 0.1 mg to about 500 mg of the multifunctional molecule. In some embodiments, the pharmaceutical composition comprises from about 0.5 mg to about 200 mg of the multifunctional molecule. In some embodiments, the pharmaceutical composition comprises from about 0.5 mg to about 100 mg of the multifunctional molecule. In some embodiments, the pharmaceutical composition comprises from about 1 mg to about 200 mg of the multifunctional molecule. In some embodiments, the pharmaceutical composition comprises from about 1 mg to about 100 mg of the multifunctional molecule.

In some embodiments, the pharmaceutical composition comprises about 1 mM to about 200 mM, about 2 mM to about 100 mM, about 10 mM to about 50 mM, about 15 mM to about 25 mM, or about 20 mM L-histidine/L-histidine monohydrochloride buffer.

In some embodiments, the pharmaceutical composition comprises about 1% (w/v) to about 20% (w/v), about 2% (w/v) to about 15% (w/v), 5% (w/v) to about 12% (w/v), about 6% (w/v) to about 10% (w/v), about 8% (w/v) sucrose.

In some embodiments, the pharmaceutical composition comprises about 0.001% (w/v) to about 0.1% (w/v), about 0.002% (w/v) to about 0.08% (w/v), 0.005% (w/v) to about 0.06% (w/v), about 0.008% (w/v) to about 0.04% (w/v), about 0.01% (w/v) to about 0.03% (w/v), about 0.02% (w/v) polysorbate-80.

In some embodiments, the pharmaceutical composition comprises the multifunctional molecule at a concentration of about 0.5 mg/mL to about 200 mg/mL, about 1 mg/mL to about 100 mg/mL, about 2 mg/mL to about 80 mg/mL, about 4 mg/mL to about 50 mg/mL, about 6 mg/mL to about 20 mg/mL, about 8 mg/mL to about 12 mg/mL, or about 10 mg/mL.

In some embodiments, the pharmaceutical composition comprises one or more of L-histidine/L-histidine monohydrochloride buffer, sucrose, or polysorbate.

In some embodiments, the multifunctional molecule comprises a first polypeptide, a second polypeptide, and a third polypeptide; wherein the first polypeptide, the second polypeptide and the third polypeptide are non-contiguous, wherein the first polypeptide comprises a first portion of a dimerization module linked to a first portion of the TCRβV6-binding moiety comprising a VH of the TCRβV6-binding moiety; the second polypeptide comprises a second portion of the dimerization module, wherein the IL-2 or functional fragment or functional variant thereof is covalently linked to the second polypeptide; and the third polypeptide comprises a second portion of the TCRβV6-binding moiety comprising a VL of the TCRβV6-binding moiety.

In some embodiments, the first polypeptide comprises a sequence with at least 80% sequence identity to any one of SEQ ID NOs: 3517, 4000, 4004, 4006, 4008, 4010, 4011, 4014, 4016 and 4018, the second polypeptide comprises a sequence with at least 80% sequence identity to any one of SEQ ID NOs: 3521, 4002, 4030, 4007, 4003, 4013 and 4015 and the third polypeptide comprises a sequence with at least 80% sequence identity to any one of SEQ ID NOs: 3518, 4005, 4009, 4012 and 4017.

In some embodiments, the multifunctional molecule comprises a first polypeptide and a second polypeptide; wherein the first polypeptide and the second polypeptide are non-contiguous, wherein the TCRβV6-binding moiety comprises a heavy chain variable domain (VH) and a light chain variable domain (VL), or a single domain antibody, wherein the first polypeptide comprises a first portion of a dimerization module linked to the TCRβV6-binding moiety; and the second polypeptide comprises a second portion of the dimerization module, wherein the IL-2 or functional fragment or functional variant thereof is covalently linked to the second polypeptide.

In some embodiments, the first polypeptide comprises a sequence with at least 80% sequence identity to any one of SEQ ID NOs: 4019, 4021, 4023, 4025 and 4027, and the second polypeptide comprises a sequence with at least 80% sequence identity to any one of SEQ ID NOs: 4020, 4022, 4024, 4026 and 4028.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows an overview of treatment and assessment plans for this study.

FIG. 2 shows Predicted Effect on Vβ6/Vβ10 CD8+ T Cell Expansion (Frequency) in Humans After a Single 1-hour IV Infusion of Compound 1 Across Various Dose Levels. Body weight of 70 kg. Solid line—median, shaded area—5th and 95th percentile from 1000 simulated individuals, dashed line—20% increase over baseline.

FIG. 3 shows Integrated Summary of Compound 1 Pharmacology, MABEL Estimates, and Cmax Predictions for Phase 1 Dose Escalation. MABEL estimate 1: First diamond from left: EC20 of in vitro human Vβ6/Vβ10 CD8+ T cell expansion (0.7 nM) and second diamond from left: Cmax at the modelled ED20 dose from monkey studies of IV (0.91 nM). MABEL estimate 2: Predicted human Cmax (7.98 nM) associated with starting dose of 0.04 mg/kg at which 20% increase in Vβ6/Vβ10 CD8+ T cell expansion over baseline is predicted in ≥95% patients. *Moderate hunched posture, reduced activity, and reduced appetite observed in monkeys dosed with 1 mg/kg IV Compound 1 4-5 days post infusion in repeat dose GLP toxicology study, with fever, diarrhea, and mild dehydration in monkeys dosed with 1.5 mg/kg Compound 1 in single dose non-GLP pharmacology study. These signs resolved by Day 8-9 post dosing. Sporadic and transient hunched posture and reduced activity observed at 0.5 mg/kg dose level in repeat dose non-GLP monkey pharmacology studies.

FIG. 4 shows Phase 1 Trial Design.

FIG. 5 shows that a multifunctional molecule containing an anti-TCRβVβ6 binding domain and an IL-2 domain (Compound 1) increases TCR signaling as measured by pERK level compared to a multifunctional molecule containing an non-TCR binding domain and IL-2 control and a multifunctional molecule containing two non-TCR binding domains control.

FIG. 6 shows potent single-agent activity of a murine surrogate bispecific antibody (BsAb) of mSTAR with durable response in various tumor models including PD-1 refractory models.

FIG. 7 shows that mSTAR leads to potent tumor regressions in EMT6 model.

FIGS. 8A-8B show that mSTAR remodels tumor infiltrating lymphocytes (TILs), e.g., expansion of Vβ CD8+/CD4+ T effector memory (TEM) cells and Central memory T (TCM) cells. FIG. 8A shows scRNAseq analysis of EMT6 TIL. FIG. 8B shows scRNAseq analysis of TIL subtypes.

FIG. 9 shows that mSTAR induces a novel TEM phenotype. For each violin plot, Vehicle is on the left and mSTAR is on the right.

FIGS. 10A-10B show that mSTAR induces an increase in TCR diversity in TILs. FIG. 10A shows that mSTAR increases Vβ TIL Clonal Diversity. FIG. 10B shows large increase in unique CDR3 transcripts in TILs treated with mSTAR.

FIG. 11 shows that Compound 1 induced expansion of Vβ6 CD8+ T cells in blood of monkeys with minimal Treg.

FIG. 12 shows that Compound 1 induces ex vivo expansion of patient TILs and killing of refractory autologous tumors as compared to pembrolizumab.

FIG. 13 shows mSTAR promotes “functional memory” & long-term protection as a result of Vβ CD8+ T cells.

FIG. 14 shows that Compound 1 and a multifunctional molecule containing an non-TCR binding domain and IL-2 control increase IL-2R signaling as measured by pSTAT5 level compared to an isotype control.

FIG. 15 shows the prevalence of Vβ6 TCR T cells in isolated TILs and PBMCs from cancer patients (n=43) and healthy donors (n=20). For each cancer type, left bar denotes Vβ6-5⁺ TILs and right bar denotes Vβ6-5⁺ PB.

FIG. 16 is a schematic depicting exemplary embodiments of a multifunctional molecule comprising a TCRβV-binding moiety and a cytokine polypeptide (e.g., IL2) as described herein.

FIG. 17 shows that Compound 1 bound similarly as single-arm anti-Vβ6/Vβ10 controls to human CD4+ and CD8+ T cells.

FIG. 18 shows IL-2 bioreactivity as pSTAT5 activity across Compound 1, RSV-IL2, and rhIL2.

FIG. 19 shows a series of FACS plots demonstrating Pan-T cells or Vβ6-5 sorted T cells with high CD25 levels (CD25^{Hi}) or low CD25 levels (CD25^{Lo}). Pan T cells and Vβ6-5 sorted T cells were expanded with anti-CD3/CD28 beads, supplemented with recombinant human IL-2. Aliquot of expanded Vβ6-5+T cells was stained for Vβ6-5 to confirm purity using PE anti-Vβ6-5. Aliquots of expanded pan T cells and Vβ6-5 T cells with CD25^{hi} expression were allowed to rest for 3 days to allow down-regulation of CD25 for a phenotype with CD25^{lo} expression.

FIG. 20 shows that in stimulated and unstimulated-sorted T cell populations (pan-T cells or Vβ6-5 sorted T cells comprising either high or low levels of CD25 following anti-CD3/CD28 stimulation or resting of cells, respectively), Compound 1 bound in a Vβ TCR-dependent manner with greater avidity to Vβ6 CD25Hi and CD25Lo T cells.

FIG. 21 shows gene expression analysis charts for TRBV-specific T cells lines P12-Ichikawa and HSB-2 via Nanostring and CD25 expression via FACS.

FIG. 22 shows dose-dependent binding of Compound 1 to P12-Ichikawa and HSB-2 T cell lines.

FIG. 23 shows a pie chart of the relative frequencies of human T cell Vβ6 (purple) and Vβ10 (blue) transcripts pre- and post-stimulation with Compound 1 (n=3).

FIG. 24 shows in vitro TCR sequencing. PBMCs were incubated with Compound 1 for 5 days and T cells were sequenced for TCR R chain V (TRBV) genes. Compared to unstimulated T cells (grey), Compound 1 selectively expanded T cells bearing TRBV6-1, TRBV6-2, TRBV6-3, TRBV6-5, and TRBV10-3. (n=3 independent donors).

FIG. 25 shows a series of FACS plots showing the expansion of Vβ6/Vβ10 T cells over 8 days.

FIG. 26 shows a series of graphs exhibiting activation of CD4+ and CD8+ T cells as assessed by CD25 expression following stimulation with Compound 1, or anti-RSV Fab x IL2 control, or anti-Vβ6/Vβ10 control in solution.

FIG. 27 shows direct cell counts of CD4+ and CD8+ T cells following stimulation with Compound 1. Mean values±SEM, n=4.

FIG. 28 shows purified T cells incubated with Compound 1 and competing concentrations of soluble IL2R, Vβ6-5 antigen, or a mixture of both. Addition of the competitors elicited a dose dependent inhibition of T cell activation.

FIG. 29 shows a series of FACS plots demonstrating differentiation of Vβ6/Vβ10 CD8+ T cells mediated by Compound 1 (10 nM) in comparison to Isotype and the controls (RSV-IL2 and anti-Vβ6/Vβ10). First column represents Vβ6/Vβ10 T cells, middle column represents Naïve T cells (CD95−), and right column represents central memory. Asterisk denotes plate bound. Treatment with Compound 1, or the controls lasted for 7 days before the analysis.

FIG. 30 shows a series of graphs depicting the summary analysis of FIG. 23 for CD4+(left) and CD8+ (right) Vβ6/Vβ10 central memory T cells. Squares denote PBMCs, n=3, and circles denote purified T cells, n=2.

FIGS. 31A-31C show assessment of TCR and IL-2R signaling using phospho-SLP76, phospho-ERK and phospho-STAT5 quantification. FIG. 31A shows that Compound 1 increased pSLP76 levels in purified CD8+ T cells compared to those from control molecules. FIG. 31B shows that Compound 1 increased TCR signaling as measured by pERK level compared to single arm controls. FIG. 31C shows that Compound 1 and IL-2 control increased IL-2 signaling as measured by STAT5 level compared to anti-TCRβVβ6/Vβ10 monovalent antibody.

FIG. 32 is a bar graph showing the percentage of CD8+ T cells triple positive for CD25, IFNγ, and Granzyme B after no treatment, treatment with Compound 1, or controls.

FIG. 33 shows activation of murine splenocytes cultured with a dose-titration of mSTAR, RSV-IL2, and isotype control. Top: CD4+ T cells. Bottom: CD8+ T cells. Data showing n=1 of 3 independent donors.

FIG. 34 shows the pharmacokinetic profile of mSTAR after single 0.5, 1.0, or 1.5 mg/kg IP dose in mice.

FIG. 35 shows the pharmacodynamic profiles of Vβ13-2/3 subsets of CD8+ and CD4+ cells, and total Tregs after single 1 mg/kg IP dose in mice.

FIG. 36 shows the biodistribution of mSTAR in tumor, spleen, liver, kidney, and lung tissues of BALB/c EMT6 tumor bearing mice. Biodistribution was measured after 6-, 24-, 48-, 72- and 120-hours post dosing of 1 mg/kg of mSTAR. Data shown as mean+/−SEM.

FIG. 37 shows expansion of Vβ13 T cells in mice after 3 doses 0.5-1.5 mg/kg IP of mSTAR but not after vehicle or rhIL-2.

FIGS. 38A-38C show levels of perivascular leukocyte infiltration in mice dosed with rhIL2, PBS, or different concentrations of mSTAR. FIG. 38A shows IHC staining of lung and liver tissue demonstrating perivascular leukocyte infiltration. FIG. 38B shows quantification of perivascular CD8+ T cells from liver tissue. FIG. 38C shows quantification of perivascular CD8+ T cells from lung tissue.

FIGS. 39A-39D show changes in serum liver enzyme markers in mice dosed with rhIL2, PBS, or mSTAR. FIG. 39A shows aspartate aminotransferase, FIG. 39B shows alanine transaminase, FIG. 39C shows alkaline phosphatase, and FIG. 39D shows albumin.

FIGS. 40A-40B shows effects of mSTAR at various doses on measurements of tumor volume and mouse survival. Increasing concentrations of mSTAR were administered IP once per week for four treatments. Triangles indicate dosing intervals. n=8 per group, **** p<0.0001, *** p<0.001, ** p<0.01, ns=not significant. FIG. 40A shows that mSTAR, at 0.3 mg/kg, 0.5 mg/kg and 1.0 mg/kg, led to potent tumor regressions in EMT6 model. FIG. 40B shows potent single-agent activity of mSTAR at 1.0 mg/kg with durable response in an EMT6 model.

FIGS. 41A-41B shows effects of single-dose mSTAR at 1.0 mg/kg on measurements of tumor volume and mouse survival. Triangle indicates time of dosing. n=8 per group, ** p<0.01, * p<0.05, ns=not significant. FIG. 41A shows that mSTAR led to potent tumor regressions in EMT6 model. FIG. 41B shows potent activity of single-dose mSTAR with durable response in an EMT6 model.

FIG. 42 shows tumor growth curves of mSTAR-treated mice. Studies were performed in randomized mice with tumor volumes of 80-150 mm³. For all models except RM1, mice were dosed for 3-4 weeks with a weekly (QW) dosing of 1 mg/kg and survival was determined based on 2000 mm³ tumor volume end point. For RM1, mice were given 1.5 mg/kg for twice weekly (2QW) doses.

FIG. 43 shows Kaplan-Meier survival curves of treated mice. Studies were performed in randomized mice with tumor volumes of 80-150 mm³. For all models except RM1, mice were dosed for 3-4 weeks with a weekly (QW) dosing of 1 mg/kg and survival was determined based on 2000 mm³ tumor volume end point. For RM1, mice were given 1.5 mg/kg for twice weekly (2QW) doses.

FIG. 44 shows Kaplan-Meier survival curves of treated mice. Studies were performed in randomized mice with tumor volumes of 80-150 mm³. For MC38 and Renca tumor models, mSTAR-treated mice were dosed for 3 weeks with a weekly dosage of 1 mg/kg. For RM1, mice were given 1.5 mg/kg once per week. For anti-PD1, mice were administered 10 mg/kg anti-mouse PD1 twice per week for a total of five treatments. Survival was determined based on 2000 mm³ tumor volume end point.

FIG. 45 shows that mSTAR led to potent tumor regressions in EMT6 model as compared to single-arm controls (n=8, mean+/−SEM, p<0.0001).

FIG. 46 shows IHC staining of EMT6 tumors for CD8 and Granzyme B expression.

FIG. 47 shows immunophenotyping of CD8+ TILs isolated from EMT6 mice after tumor transplant. Mean values±SEM, n=4. **** p<0.0001, *** p<0.001. ** p<0.01, ns=not significant.

FIG. 48 shows immunotyping of NK cells and B cells isolated from EMT6 mice after tumor transplant. Mean values t SEM, n=4. ns=not significant.

FIG. 49 shows immunophenotyping of non-CD8+ TILs isolated from EMT6 mice after tumor transplant. Mean values±SEM, n=4. *** p<0.001. ** p<0.01. * p<0.05, ns=not significant.

FIG. 50 shows immunophenotyping of CD8+ TILs isolated from EMT6 mice after tumor transplant, comparing RSV F(ab)₂x(IL-2)₂ and mSTAR administration. Mean values±SEM, n=4. **** p<0.0001, *** p<0.001, ** p<0.01, * p<0.05, ns=not significant.

FIG. 51 shows that mSTAR 1.0 mg/kg led to potent tumor regressions in EMT6 model. Depletion of Vβ13 T cells abolished the anti-tumor activity of mSTAR.

FIG. 52 shows the results of a tumor rechallenge study. Left: while the EMT6 tumors were rejected, CT26 tumors grew, suggesting that the memory response against EMT6 tumors likely mediated through mSTAR treatment had been established. Right: depletion of CD8+ T cells prior to rechallenge resulted in EMT6 tumor growth.

FIG. 53 shows a UMAP plot illustrating results from single cell analysis of the EMT6 TIL transcriptome for CD4+ and CD8+ gene expression.

FIG. 54 shows single cell RNAseq analysis of single CD4+ or CD8+ TILS isolated from EMT6 mice on day 14 post tumor implant following a single dose of mSTAR (right) or vehicle (left) from n=5 mice pooled per group.

FIG. 55 shows that Vβ13 T cells were labelled positive based on gene expression of TRBV13-2 and TRBV13-3.

FIG. 56 shows Vβ13 T cells across the UMAP plot of FIG. 54 inferred from expression of TRBV13-2 and TRBV13-3 transcripts.

FIG. 57 shows quantification of cell subsets in EMT6 TILs from mice treated with vehicle or mSTAR. For each cell subset, the top bar denotes control (CTRL) and bottom bar denotes mSTAR.

FIG. 58 shows quantification of Vβ13 T cells and TIL subtypes.

FIG. 59 shows a heatmap illustrating the number of differentially expressed genes (DEGs) in TIL when comparing Vβ13+ subsets from mSTAR versus vehicle-treated mice.

FIGS. 60A-60D show a series of volcano plots of differentially expressed genes between targeted-Vβ13 T cell subsets by mSTAR treatment and vehicle control-treated groups.

FIG. 61 shows a heatmap of differentially expressed genes in response to mSTAR treatment compared to vehicle across indicated T cell subsets. Expression values are scaled for each gene.

FIG. 62 shows that mSTAR induced a novel CD8-T_{EM}, phenotype. For each plot, left violin denotes vehicle and right violin denotes mSTAR.

FIG. 63 shows a series of heatmaps of differentially expressed genes identified as distinct with m STAR treatment compared against IL-2, anti-PD-1 and anti-PD-1-IL-2 mutein treatments from published studies.

FIG. 64 shows a series of Venn diagrams showing the number of overlapping genes between distinct Compound 1 genes and genes that are significantly and differentially expressed from vehicle and IL-2, anti-PD-1 and anti-PD-1-IL-2 mutein treatments.

FIG. 65 shows a series of violin plots showing TCR signaling repressor genes after treatment in CD8 effector T cell subsets and CD8 ‘Better effector’ T cells of published studies.

FIG. 66 shows clonal diversity within each TRBV gene from TILs obtained from EMT6 mice treated with vehicle (top) or mSTAR (bottom).

FIG. 67 shows that mSTAR induced an increase in TCR diversity in TILs. Top: Treatment with mSTAR increased clonal diversity in targeted Vβ13 T cells but not non-targeted Vβ5 T cells. Bottom: bubble plots show a large increase in unique CDR3 transcripts in Vβ13 TILs treated with mSTAR compared to those treated with vehicle.

FIG. 68 shows quantification of clonal diversity of TILs between vehicle and mSTAR-treated mice using the inverse Simpson index.

FIG. 69 shows single cell RNAseq analysis of Vβ13 and Vβ5 T cells in vehicle and mSTAR-treated mice.

FIG. 70 shows IFN-γ intracellular FACS staining from an ex vivo tumor antigen recall assay in Vβ13 CD8+ T cell splenocytes isolated from EMT6-tumor bearing mice that were treated with 0, 0.5, 1, or 1.5 mg/kg mSTAR.

FIG. 71 shows clonal diversity in TILs between MC38 mice treated with vehicle, mSTAR, or anti-RSV-IL-2. Left: clonal sizes. Right: inverse Simpson index for diversity.

FIG. 72 shows similar data of FIG. 71 for CT26 tumor mice.

FIG. 73 shows isolated tumor-infiltrating lymphocytes (TILs) from CT26 tumor bearing mice treated with mSTAR stained for tetramers recognizing the tumor-rejection antigen AH1/gp70 within Vβ13+CD8+ and Vβ13− CD8+ T cells.

FIG. 74 shows pharmacokinetic profile (serum concentration over time) of single dose Compound 1 administered IV in cynomolgus monkeys.

FIG. 75 shows T cell frequency in blood following a single IV dose 1 mg/kg of Compound 1. n=3 monkeys.

FIG. 76 shows serum soluble CD25 levels in monkeys administered a single IV dose 0.5 mg/kg of Compound 1. Mean values±SEM, n=6.

FIG. 77 shows serum levels of IFNγ, TNFα, and IL-6 in monkeys administered a single IV dose 0.5 mg/kg of Compound 1. Mean values±SEM, n=6.

FIG. 78 shows serum levels of IL-5 and cosinophil counts in monkeys administered a single IV dose 1 mg/kg of Compound 1. Mean values±SEM, n=3.

FIGS. 79A-79D show serum levels of liver enzyme markers in monkeys following a single IV dose of Compound 1. FIG. 79A shows aspartate aminotransferase, FIG. 79B shows alanine transaminase, FIG. 79C shows alkaline phosphatase, and FIG. 79D shows albumin.

FIG. 80 shows that Compound 1 induced ex vivo expansion of patient TILs and killing of refractory autologous tumors as compared to pembrolizumab. Autologous T cells were incubated with Compound 1 at 3 μg/ml, pembrolizumab at 10 μg/ml, or isotype control at 3 μg/ml, for 5 days.

FIG. 81 shows the frequency of Vβ6/Vβ10 T cells across the four organoid models.

FIG. 82 shows Compound 1-mediated killing of human tumor organoids generated from primary, patient-derived tissue from colorectal and NSCLC cancer patients. Vertical bars represent percentage of organoid area reduced relative to isotype control following incubation of organoids with Compound 1 and autologous TILs. Mean values±SEM, n=4, **** p<0.0001, ** p<0.01, ns=not significant.

FIG. 83 shows dose-dependent cancer organoid killing with Compound 1 in a NSCLC PDX model. Reduction of organoid size was not observed for the IL-2 control molecule.

FIG. 84 shows Compound 1-mediated cx vivo activation of HPV-16 specific T cells in PBMCs of healthy donors. Mean values±SEM, n=7, * p<0.05, ns=not significant. PBMCs were treated for 1 hour with 1 nM Compound 1, isotype control, or media, and then stimulated with HPV-16 peptides or a negative control and stained for intracellular expression of IFNγ, TNFα, IL-2, and CD107a. 1001381FIG. 85 shows Compound 1-mediated ex vivo expansion of tumor antigen specific T cells targeting HPV-16. PBMCs were obtained from a healthy donor and treated for 1 hour with 1 nM Compound 1, anti-TCR V16/Vβ10 antibody, isotype control, or media, and then stimulated for 7 days with HPV-16 overlapping 15-mer peptides or a negative control.

FIG. 86 shows Compound 1-mediated cx vivo activation of HPV-16 specific T cells in PBMCs of a cervical cancer patient. PBMCs were treated for 1 hour with 1 nM Compound 1, isotype control, or media, and then stimulated with HPV-16 peptides or a negative control and stained for intracellular expression of IFNγ, TNFα, IL-2, and CD107a.

FIGS. 87A-87B show two SDS-PAGE gels and chromatography size-exclusion chromatography analysis plots for Compound 1 (FIG. 87A) and mSTAR (FIG. 87B).

### DETAILED DESCRIPTION

### Definition

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

As used in this specification and the appended claims, the singular forms “a.” “an,” and “the” include plural referents unless the content clearly dictates otherwise. The use of the words “a” or “an” when used in conjunction with the term “comprising” herein may mean “one,” but it is also consistent with the meaning of “one or more,” “at least one,” and “one or more than one.”

It should also be noted that the term “or” is generally employed in its sense including “and/or” unless the content clearly dictates otherwise.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

The term “about” when referring to a measurable value such as an amount, a temporal duration, and the like, is meant to encompass variations of ±20% or in some instances ±10%, or in some instances ±5%, or in some instances 1%, or in some instances ±0.1% from the specified value, as such variations are appropriate to perform the disclosed methods. As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

The term “acquire” or “acquiring” as the terms are used herein, refer to obtaining possession of a physical entity (e.g., a sample, a polypeptide, a nucleic acid, or a sequence), or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Directly acquiring a value includes performing a process that includes a physical change in a sample or another substance, e.g., performing an analytical process which includes a physical change in a substance, e.g., a sample.

“Antibody molecule” as used herein refers to a protein, e.g., an immunoglobulin chain or fragment thereof, comprising at least one immunoglobulin variable domain structure and/or sequence. An antibody molecule encompasses antibodies (e.g., full-length antibodies) and antibody fragments. In some embodiments, an antibody molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)₂ fragments, and single chain variable fragments (scFvs). In some embodiments, the antibody molecule is an antibody mimetic. In some embodiments, the antibody molecule is, or comprises, an antibody-like framework or scaffold, such as, fibronectins, ankyrin repeats (e.g., designed ankyrin repeat proteins (DARPins)), avimers, affibody affinity ligands, anticalins, or affilin molecules.

The term “human-like antibody molecule” as used herein refers to a humanized antibody molecule, human antibody molecule or an antibody molecule having at least 95% sequence identity with a non-murine germline framework region, e.g., FR1, FR2, FR3 and/or FR4. In some embodiments, the human-like antibody molecule comprises a framework region having at least 95% sequence identity to a human germline framework region, e.g., a FR1, FR2, FR3 and/or FR4 of a human germline framework region. In some embodiments, the human-like antibody molecule is a recombinant antibody. In some embodiments, the human-like antibody molecule is a humanized antibody molecule. In some embodiments, the human-like antibody molecule is human antibody molecule. In some embodiments, the human-like antibody molecule is a phage display or a yeast display antibody molecule. In some embodiments, the human-like antibody molecule is a chimeric antibody molecule. In some embodiments, the human-like antibody molecule is a CDR grafted antibody molecule.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

In embodiments, an antibody molecule is monospecific, e.g., it comprises binding specificity for a single epitope. In some embodiments, an antibody molecule is multifunctional, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

“Antigen” (Ag) as used herein refers to a molecule that can provoke an immune response, e.g., involving activation of certain immune cells and/or antibody generation. Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

The “antigen-binding site,” or “binding portion” of an antibody molecule refers to the part of an antibody molecule. e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4.

As used herein, an “immune cell” refers to any of various cells that function in the immune system, e.g., to protect against agents of infection and foreign matter. In embodiments, this term includes leukocytes, e.g., neutrophils, eosinophils, basophils, lymphocytes, and monocytes. Innate leukocytes include phagocytes (e.g., macrophages, neutrophils, and dendritic cells), mast cells, cosinophils, basophils, and natural killer cells. Innate leukocytes identify and eliminate pathogens, either by attacking larger pathogens through contact or by engulfing and then killing microorganisms, and are mediators in the activation of an adaptive immune response. The cells of the adaptive immune system are special types of leukocytes, called lymphocytes. B cells and T cells are important types of lymphocytes and are derived from hematopoietic stem cells in the bone marrow. B cells are involved in the humoral immune response, whereas T cells are involved in cell-mediated immune response. The term “immune cell” includes immune effector cells.

“Immune effector cell,” as that term is used herein, refers to a cell that is involved in an immune response, e.g., in the promotion of an immune effector response. Examples of immune effector cells include, but are not limited to, T cells, e.g., alpha/beta T cells and gamma/delta T cells, B cells, natural killer (NK) cells, natural killer T (NK T) cells, and mast cells.

The term “effector function” or “effector response” refers to a specialized function of a cell. Effector function of a T cell, for example, may be cytolytic activity or helper activity including the secretion of cytokines.

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature. An isolated polynucleotide (ribonucleic acid (RNA), deoxyribonucleic acid (DNA)), or polypeptide is free of the genes/nucleic acids or sequences/amino acids that flank it in its naturally-occurring state.

The compositions and methods of the present invention encompass polypeptides and nucleic acids having the sequences specified, or sequences substantially identical or similar thereto, e.g., sequences at least 80%, 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 80%, 85%, 90%, 91%, 92%, 93%, 94%, 95%, 96%, 97%, 98% 99%, 99.5%, 99.9%, or 100% sequence identity to a reference sequence, e.g., a sequence provided herein. In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 80%, 85%, 90%, 91%, 92%, 93% 94%, 95%, 96%, 97%, 98% 99%, 99.5%, 99.9%, or 100% sequence identity to a reference sequence, e.g., a sequence provided herein.

The term “variant” refers to a polypeptide that has a substantially identical amino acid sequence to a reference amino acid sequence, or is encoded by a substantially identical nucleotide sequence. In some embodiments, the variant is a functional variant. In some embodiments, a TCRβV variant can bind to TCRα and form a TCR α:β complex.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

Calculations of homology or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4. The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecule of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

As used herein, the term “molecule” as used in, e.g., antibody molecule, cytokine molecule, receptor molecule, includes full-length, naturally-occurring molecules, as well as variants, e.g., functional variants (e.g., truncations, fragments, mutated (e.g., substantially similar sequences) or derivatized form thereof), so long as at least one function and/or activity of the unmodified (e.g., naturally-occurring) molecule remains.

As used herein, the term “mutation” refers to an alteration in the nucleotide sequence of the genome of an organism, virus, or extrachromosomal DNA. In some embodiments, the mutation may be a large-scale mutation, such as amplifications (or gene duplications) or repetitions of a chromosomal segment, deletions of large chromosomal regions, chromosomal rearrangements (e.g., chromosomal translocations, chromosomal inversions, non-homologous chromosomal crossover, and interstitial deletions), and loss of heterozygosity. In some embodiments, the mutation may be a small-scale mutation, such as insertions, deletions, and substitution mutations. As used herein, the term “substitution mutation” refers to the transition that exchange a single nucleotide for another.

“Interleukin-2” also known as IL2, IL-2, IL 2, TCGF, lymphokine, and interleukin 2, as referred to herein, includes any of the recombinant or naturally-occurring forms of IL-2 or variants or homologs thereof that have or maintain IL-2 activity (e.g., at least 40% 50%, 60%, 70%, 80%, 90%, 95%, 96%, 97%, 98%, 99% or 100% activity). In some aspects, the variants or homologs have at least 40%, 45%, 50%, 55%, 60%, 65%, 70%, 75%, 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring IL-2. In some embodiments. IL-2 is substantially identical to the protein identified by the UniProt reference number P60568 or a variant or homolog having substantial identity thereto.

### Anti-TCRβV Antibodies

### Human T Cell Receptor (TCR) Complex

TCR is a disulfide-linked membrane-anchored heterodimeric protein normally consisting of the highly variable alpha (α) and beta (β) chains expressed as part of a complex with the invariant CD3 chain molecules. TCR on αβ T cells is formed by a heterodimer of one alpha chain and one beta chain. Each alpha or beta chain consists of a constant domain and a highly variable domain classified as the Immunoglobulin superfamily (IgSF) fold. The TCRβV chains can be further classified into 30 subfamilies (TRBV1-30). Despite their high structural and functional homology, the amino acid sequence homology in the TRBV genes is very low. Only 4 amino acids out of approximately 95 are identical while 10 additional amino acids are conserved among all subfamilies. Nevertheless, TCRs formed between alpha and beta chains of highly diverse sequences show a remarkable structural homology and elicit a similar function, e.g., activation of T cells.

T cell receptors (TCR) can be found on the surface of T cells. TCRs recognize antigens, e.g., peptides, presented on, e.g., bound to, major histocompatibility complex (MHC) molecules on the surface of cells, e.g., antigen-presenting cells. TCRs are heterodimeric molecules and can comprise an alpha chain, a beta chain, a gamma chain or a delta chain. TCRs comprising an alpha chain and a beta chain are also referred to as TCRαβ. The TCR beta chain consists of the following regions (also known as segments): variable (V), diversity (D), joining (J) and constant (C) (see Mayer G, and Nyland J. (2010) Chapter 10: Major Histocompatibility Complex and T-cell Receptors-Role in Immune Responses. In: Microbiology and Immunology on-line, University of South Carolina School of Medicine). The TCR alpha chain consists of V. J and C regions. The rearrangement of the T-cell receptor (TCR) through somatic recombination of V (variable), D (diversity), J (joining), and C (constant) regions is a defining event in the development and maturation of a T cell. TCR gene rearrangement takes place in the thymus.

TCRs can comprise a receptor complex, known as the TCR complex, which comprises a TCR heterodimer comprising of an alpha chain and a beta chain, and dimeric signaling molecules, e.g., CD3 co-receptors, e.g., CD3δ/ε, and/or CD3γ/ε.

As used herein, the term “T cell receptor beta variable chain” or “TCRβV,” refers to an extracellular region of the T cell receptor beta chain which comprises the antigen recognition domain of the T cell receptor. The term TCRβV includes isoforms, mammalian, e.g., human TCRβV, species homologs of human and analogs comprising at least one common epitope with TCRβV. Human TCRβV comprises a gene family comprising subfamilies including, but not limited to: a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, a TCRβ V29 subfamily, a TCRβ V1 subfamily, a TCRβ V17 subfamily, a TCRβ V21 subfamily, a TCRβ V23 subfamily, or a TCRβ V26 subfamily, as well as family members of said subfamilies, and variants thereof (e.g., a structural or functional variant thereof). In some embodiments, the TCRβ V6 subfamily comprises: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, TCRβV comprises TCRβ V6-5*01, or a variant thereof, e.g., a variant having 85%, 90%, 95%, 99% or more identity the naturally-occurring sequence. TCRβ V6-5*01 is also known as TRBV65; TCRBV6S5; TCRβV13S1, or TCRβ V13.1. The amino acid sequence of TCRβ V6-5*01, e.g., human TCRβ V6-5*01, is known in that art, e.g., as provided by IMGT ID L36092. In some embodiments, TCRβ V6-5*01 is encoded by the nucleic acid sequence of SEQ ID NO: 43, or a sequence having 85%, 90%, 95%, 99% or more identity thereof. In some embodiments, TCRβ V6-5*01 comprises the amino acid sequence of SEQ ID NO: 44, or a sequence having 85%, 90%, 95%, 99% or more identity thereof.

SEQ ID NO: 43
ATGAGCATCGGCCTCCTGTGCTGTGCAGCCTTGTCTCTCCTGTGG
GCAGGTCCAGTGAATGCTGGTGTCACTCAGACCCCAAAATTCCAG
GTCCTGAAGACAGGACAGAGCATGACACTGCAGTGTGCCCAGGAT
ATGAACCATGAATACATGTCCTGGTATCGACAAGACCCAGGCATG
GGGCTGAGGCTGATTCATTACTCAGTTGGTGCTGGTATCACTGAC
CAAGGAGAAGTCCCCAATGGCTACAATGTCTCCAGATCAACCACA
GAGGATTTCCCGCTCAGGCTGCTGTCGGCTGCTCCCTCCCAGACA
TCTGTGTACTTCTGTGCCAGCAGTTACTC
SEQ ID NO: 44
MSIGLLCCAALSLLWAGPVNAGVTQTPKFQVLKTGQSMTLQCAQD
MNHEYMSWYRQDPGMGLRLIHYSVGAGITDQGEVPNGYNVSRSTT
EDFPLRLLSAAPSQTSVYFCASSY

### TCR Beta V (TCRβV)

Diversity in the immune system enables protection against a huge array of pathogens. Since the germline genome is limited in size, diversity is achieved not only by the process of V(D)J recombination but also by junctional (junctions between V-D and D-J segments) deletion of nucleotides and addition of pseudo-random, non-templated nucleotides. The TCR beta gene undergoes gene arrangement to generate diversity.

The TCR V beta repertoire varies between individuals and populations because of, e.g., 7 frequently occurring inactivating polymorphisms in functional gene segments and a large insertion/deletion-related polymorphism encompassing 2 V beta gene segments.

Provided herein are, inter alia, antibody molecules and fragments thereof, that bind, e.g., specifically bind, to a human TCR beta V chain (TCR 3V), e.g., a TCRβV gene family (also referred to as a group), e.g., a TCRβV subfamily (also referred to as a subgroup), e.g., as described herein. TCR beta V families and subfamilies are known in the art, e.g., as described in Yassai et al., (2009) Immunogenetics 61(7)pp:493-502; Wei S, and Concannon P. (1994) Human Immunology 41(3) pp: 201-206. The antibodies described herein can be recombinant antibodies, e.g., recombinant non-murine antibodies, e.g., recombinant human or humanized antibodies.

The terms TCRBV, TCRβVB, TRBV, TCRβV, TCRβVβ or TRβV are used interchangeably herein and refer to a TCR beta V chain, e.g., as described herein.

In some embodiments, provided herein is an anti-TCRβV antibody molecule that binds to human TCRβV, e.g., a TCRβV family, e.g., gene family or a variant thereof. In some embodiments a TCRβV gene family comprises one or more subfamilies, e.g., as described herein, e.g., in Table 8A or Table 8B. In some embodiments, the TCRβV gene family comprises: a TCRβ V6 subfamily, or a TCRβ V10 subfamily.

In some embodiments, TCRβ V6 subfamily is also known as TCRβ V13.1. In some embodiments, the TCRβ V6 subfamily comprises: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01, or a variant thereof. In some embodiments, TCRβ V6 comprises TCRβ V6-4*01, or a variant thereof. In some embodiments, TCRβ V6 comprises TCRβ V6-4*02, or a variant thereof. In some embodiments, TCRβB V6 comprises TCRβ V6-9*01, or a variant thereof. In some embodiments, TCRβ V6 comprises TCRβ V6-8*01, or a variant thereof. In some embodiments, TCRβ V6 comprises TCRβ V6-5*01, or a variant thereof. In some embodiments, TCRβ V6 comprises TCRβ V6-6*02, or a variant thereof. In some embodiments, TCRβ V6 comprises TCRβ V6-6*01, or a variant thereof. In some embodiments, TCRβ V6 comprises TCRβ V6-2*01, or a variant thereof. In some embodiments, TCRβ V6 comprises TCRβ V6-3*01, or a variant thereof. In some embodiments, TCRβ V6 comprises TCRβ V6-1*01, or a variant thereof.

In some embodiments, TCRβ V6 comprises TCRβ V6-5*01, or a variant thereof. In some embodiments, TCRβ V6, e.g., TCRβ V6-5*01, is recognized, e.g., bound, by SEQ ID NO: 1 and/or SEQ ID NO: 2. In some embodiments, TCRβ V6, e.g., TCRβ V6-5*01, is recognized, e.g., bound, by SEQ ID NO: 9 and/or SEQ ID NO: 10. In some embodiments, TCRβ V6 is recognized, e.g., bound, by SEQ ID NO: 9 and/or SEQ ID NO: 11.

In some embodiments, the TCRβ V10 subfamily comprises: TCRβ V10-1*01, TCRβ V10-1*02, TCRβ V10-3*01 or TCRβ V10-2*01, or a variant thereof.

Exemplary amino acid sequences for TCRβV subfamily members can be found on the ImMunoGeneTics Information System website: http://www.imgt.org/, or in a similar resource.

### Anti-TCRβV Antibodies

Current bispecific constructs designed to redirect T cells to promote tumor cell lysis for cancer immunotherapy typically utilize antibody fragments (Fab, scFv, VH, single domain antibody, etc.) that are derived from monoclonal antibodies (mAb) directed against the CD3e subunit of the T cell receptor (TCR). However, there are limitations to this approach which may prevent the full realization of the therapeutic potential for such bispecific constructs. Previous studies have shown that even low “activating” doses of anti-CD3e mAb can cause long-term T cell dysfunction and exert immunosuppressive effects. In addition, anti-CD3e mAbs have been associated with side effects that result from massive T cell activation. The large number of activated T cells secrete substantial amounts of cytokines, the most important of which is Interferon gamma (IFNγ). This excess amount of IFNγ in turn activates macrophages which then overproduce proinflammatory cytokines such as IL-1beta, IL-6, IL-10 and TNF-alpha, causing a “cytokine storm” known as the cytokine release syndrome (CRS) (Shimabukuro-Vomhagen et al., J Immunother Cancer. 2018 Jun. 15; 6(1):56, herein incorporated by reference in its entirety). Thus, the need exists for developing antibodies that are capable of binding and activating only a subset of effector T cells, e.g., to re-duce the CRS and/or neurotoxicity (NT).

Described herein are molecules targeting the TCRβV chain of TCR and methods thereof. Without wishing to be bound by theory, such molecules arm capable of binding, activating, and/or expanding only a subset of T cells, avoiding or reducing CRS and/or NT and minimizing potential immunosuppressive effects of anti-CD3 mAbs.

Described herein is a class of antibodies, i.e., anti-TCRβV antibody molecules as described herein, which despite having low sequence similarity (e.g., low sequence identity among the different antibody molecules that recognize different TCRβV subfamilies), recognize a structurally conserved, yet sequence-wise variable, region, e.g., domain, on the TCRβV protein and have a similar function (e.g., activation of T cells and a similar cytokine profile as described herein). Thus, the anti-TCRβV antibody molecules as described herein share a structure-function relationship.

Without wishing to be bound by theory, in some embodiments, the anti-TCRβV antibody molecules as described herein bind to an outward facing epitope of a TCRβV protein when it is in a complex with a TCRalpha protein. In some embodiments, the anti-TCRβV antibody molecules as described herein recognize (e.g., bind to), a domain (e.g., an epitope) on the TCRβV protein that is: (1) structurally conserved among different TCRβV subfamilies; and (2) has minimal sequence identity among the different TCRβV subfamilies. TCRβV proteins from the different TCRβV subfamilies share minimal sequence similarity. However, TCRβV proteins which have minimal sequence similarity, share a similar 3D conformation and structure.

The alignment of TCRβV amino acid sequences underscores the diversity of TCR sequences. In particular, the TRBV sequences from different subfamilies are considerably different from each other.

In some embodiments, the anti-TCRβV antibody molecules as described herein do not recognize, e.g., bind to, an interface of a TCRβV:TCRalpha complex. In some embodiments, the anti-TCRβV antibody molecules as described herein do not recognize, e.g., bind to, a constant region of a TCRβV protein. An exemplary antibody that binds to a constant region of a TCRβV region is JOVI.1 as described in Viney et al., (Hybridoma. 1992 December; 11(6):701-13). In some embodiments, the anti-TCRβV antibody molecules as described herein do not recognize, e.g., bind to, one or more (e.g., all) of a complementarity determining region (e.g., CDR1, CDR2 and/or CDR3) of a TCRβV protein.

Provided herein are, inter alia, antibody molecules directed to the variable chain of the beta subunit of TCR (TCRβV) which bind and, e.g., activate a subset of T cells. The anti-TCRβV antibody molecules as described herein result in lesser or no production of cytokines associated with CRS, e.g., IL-6, IL-1beta. IL-10 and TNF alpha; and enhanced and/or delayed production of IL-2 and IFNγ. In some embodiments, the anti-TCRβV antibodies as described herein have a cytokine profile, e.g., as described herein, which differs from a cytokine profile of a T cell engager that binds to a receptor or molecule other than a TCRβV region (“a non-TCRβV-binding T cell engager”). In some embodiments, the non-TCRβV-binding T cell engager comprises an antibody that binds to a CD3 molecule (e.g., CD3 epsilon (CD3e) molecule); or a TCR alpha (TCRα) molecule. In some embodiments, the non-TCRβV-binding T cell engager is an OKT3 antibody or an SP34-2 antibody.

In some embodiments, the anti-TCRβV antibodies as described herein result in expansion of TCRβV+ T cells, e.g., a subset of memory effector T cells known as TEMRA. Without wishing to be bound by theory, it is believed that in some embodiments, TEMRA cells can promote tumor cell lysis but not CRS. Accordingly, provided herein are methods of making said anti-TCRβV antibody molecules and uses thereof. Also described herein are multifunctional molecules, e.g., bispecific molecules comprising said anti-TCRβV antibody molecules. In some embodiments, compositions comprising anti-TCRβV antibody molecules of the present disclosure, can be used, e.g., to: (1) activate and redirect T cells to promote tumor cell lysis for cancer immuno-therapy; and/or (2) expand TCRβV+ T cells. In some embodiments, compositions comprising anti-TCRβV antibody molecules as described herein limit the harmful side-effects of CRS and/or NT, e.g., CRS and/or NT associated with anti-CD3e targeting.

In some embodiments, the anti-TCRβV antibody molecule binds to one or more of TRBV6-1, TRBV6-2, TRBV6-3. TRBV6-4, TRBV6-5, TRBV6-6, TRBV6-8 and TRBV6-9. In some embodiments, the anti-TCRβV antibody molecule is an anti-TRBV6-1, anti-TRBV6-2, anti-TRBV6-3, anti-TRBV6-4, anti-TRBV6-5, anti-TRBV6-6, anti-TRBV6-8, anti-TRBV6-9. Exemplary anti-TCRβV antibody molecules and the corresponding TCRβV subfamilies recognized by said anti-TCRβV antibody molecules are disclosed in Table 10A.

In some embodiments, the anti-TCRβV antibody molecule binds specifically to TRBV6-1. TRBV6-2, TRBV6-3, TRBV6-4, TRBV6-5, TRBV6-6, TRBV6-8 or TRBV6-9. In some embodiments, the anti-TCRβV antibody molecule binds specifically to TRBV6-1. In some embodiments, the anti-TCRβV antibody molecule binds specifically to TRBV6-2. In some embodiments, the anti-TCRβ V antibody molecule binds specifically to TRBV6-3. In some embodiments, the anti-TCRβV antibody molecule binds specifically to TRBV6-4. In some embodiments, the anti-TCRβV antibody molecule binds specifically to TRBV6-5. In some embodiments, the anti-TCRβV antibody molecule binds specifically to TRBV6-6. In some embodiments, the anti-TCRβV antibody molecule binds specifically to TRBV6-8. In some embodiments, the anti-TCRβV antibody molecule binds specifically to TRBV6-9.

In some embodiments, the light or the heavy chain variable framework (e.g., the region encompassing at least FR 1, FR2, FR3, and optionally FR4) of the anti-TCRβV antibody molecule, e.g., anti-TCRβ V6 (e.g., anti-TCRβ V6-5*01) antibody molecule can be chosen from: (a) a light or heavy chain variable framework including at least 80%, 85%, 87% 90%, 92%, 93%, 95%, 97%, 98%, or 100% of the amino acid residues from a human light or heavy chain variable framework. e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework. e.g., a light or heavy chain variable framework residue from a human mature antibody, a human germline sequence, or a human consensus sequence; (c) a non-human framework (e.g., a rodent framework): or (d) a non-human framework that has been modified, e.g., to remove antigenic or cytotoxic determinants, e.g., deimmunized, or partially humanized. In some embodiments, the light or heavy chain variable framework region (particularly FR1, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

In some embodiments, the anti-TCRβV antibody molecule, e.g., anti-TCRβ V6 (e.g., anti-TCRβ V6-5*01) antibody molecule, comprises a heavy chain variable domain having at least one, two, three, four, five, six, seven, ten, fifteen, twenty or more changes, e.g., amino acid substitutions or deletions, from an amino acid sequence of any one of A-H.1 to A-H.85, e.g., A-H.1, A-H.2 or A-H.68, e.g., the amino acid sequence of the FR region in the entire variable region, e.g., shown in SEQ ID NO: 9.

Alternatively, or in combination with the heavy chain substitutions described herein, the anti-TCRβV antibody molecule, e.g., anti-TCRβ V6 (e.g., anti-TCRβ V6-5*01) antibody molecule, comprises a light chain variable domain having at least one, two, three, four, five, six, seven, ten, fifteen, twenty or more amino acid changes, e.g., amino acid substitutions or deletions, from an amino acid sequence of any one of A-H.1 to A-H.85, e.g., A-H.1, A-H.2 or A-H.68, e.g., the amino acid sequence of the FR region in the entire variable region, e.g., shown in SEQ ID NO: 10 or SEQ ID NO: 11.

In some embodiments, the anti-TCRβV antibody molecule, e.g., anti-TCRβ V6 (e.g., anti-TCRβ V6-5*01) antibody molecule, comprises the light chain framework region 1 of A-H.1 or A-H.2.

In some embodiments, the anti-TCRβV antibody molecule, e.g., anti-TCRβ 4 V6 (e.g., anti-TCRβ V6-5*01) antibody molecule, comprises the light chain framework region 2 of A-H.1 or A-H.2.

In some embodiments, the anti-TCRβV antibody molecule, e.g., anti-TCRβ V6 (e.g., anti-TCRβ V6-5*01) antibody molecule, comprises the light chain framework region 3 of A-H.1 or A-H.2.

In some embodiments, the anti-TCRβV antibody molecule, e.g., anti-TCRβ V6 (e.g., anti-TCRβ 4 V6-5*01) antibody molecule, comprises the light chain framework region 4 of A-H.1 or A-H.2.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 1 (FR1), comprising a change. e.g., a substitution (e.g., a conservative substitution) at position 10 according to Kabat numbering. In some embodiments, the FR1 comprises a Phenylalanine at position 10, e.g., a Serine to Phenyalanine substitution. In some embodiments, the substitution is relative to a human germline light chain framework region sequence.

In some embodiments, the anti-TCRβV antibody molecule, e.g., anti-TCRβ V6 (e.g., anti-TCRβ β V6-5*01) antibody molecule, comprises a light chain variable domain comprising a framework region, e.g., framework region 2 (FR2), comprising a change, e.g., a substitution (e.g., a conservative substitution) at a position as described herein according to Kabat numbering. In some embodiments, FR2 comprises a Histidine at position 36, e.g., a substitution at position 36 according to Kabat numbering, e.g., a Tyrosine to Histidine substitution. In some embodiments, FR2 comprises an Alanine at position 46, e.g., a substitution at position 46 according to Kabat numbering, e.g., an Arginine to Alanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 3 (FR3), comprising a change, e.g., a substitution (e.g., a conservative substitution) at a position as described herein according to Kabat numbering. In some embodiments, FR3 comprises a Phenyalanine at position 87, e.g., a substitution at position 87 according to Kabat numbering, e.g., a Tyrosine to Phenyalanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 1 (FR 1) comprising a Phenylalanine at position 10, e.g., a substitution at position 10 according to Kabat numbering, e.g., a Serine to Phenyalanine substitution; (b) a framework region 2 (FR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (c) a framework region 3 (FR3) comprising a Phenylalanine at position 87, e.g., a substitution at position 87 according to Kabat numbering, e.g., a Tyrosine to Phenyalanine substitution, e.g., as shown in the amino acid sequence of SEQ ID NO: 10. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 2 (FR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (b) a framework region 3 (FR3) comprising a Phenylalanine at position 87, e.g., a substitution at position 87 according to Kabat numbering. e.g., a Tyrosine to Phenyalanine substitution, e.g., as shown in the amino acid sequence of SEQ ID NO: 11. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 1 (FR1) comprising a change, e.g., a substitution (e.g., a conservative substitution) at one or more (e.g., all) positions as described herein according to Kabat numbering; (b) a framework region 2 (FR2) comprising a change, e.g., a substitution (e.g., a conservative substitution) at one or more (e.g., all) position as described herein according to Kabat numbering and (c) a framework region 3 (FR3) comprising a change, e.g., a substitution (e.g., a conservative substitution) at one or more (e.g., all) position as described herein according to Kabat numbering. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises the heavy chain framework region 1 of A-H.1 or A-H.2. In some embodiments, the anti-TCRβV antibody molecule, e.g., anti-TCRβ V6 (e.g., anti-TCRβ V6-5*01) antibody molecule, comprises the heavy chain framework region 2 of A-H.1 or A-H.2. In some embodiments, the anti-TCRβV antibody molecule, e.g., anti-TCRβ V6 (e.g., anti-TCRβ V6-5*01) antibody molecule, comprises the heavy chain framework region 3 of A-H.1 or A-H.2. In some embodiments, the anti-TCRβV antibody molecule, e.g., anti-TCRβ V6 (e.g., anti-TCRβ V6-5*01) antibody molecule, comprises the heavy chain framework region 4 of A-H.1 or A-H.2.

In some embodiments, the anti-TCRβV antibody molecule, e.g., anti-TCRβ V6 (e.g., anti-TCRβ V6-5*01) antibody molecule, comprises a heavy chain variable domain comprising a framework region, e.g., framework region 3 (FR3), comprising a change, e.g., a substitution (e.g., a conservative substitution) at a position as described herein according to Kabat numbering. In some embodiments, FR3 comprises a Threonine at position 73, e.g., a substitution at position 73 according to Kabat numbering, e.g., a Glutamic Acid to Threonine substitution. In some embodiments, FR3 comprises a Glycine at position 94, e.g., a substitution at position 94 according to Kabat numbering, e.g., an Arginine to Glycine substitution. In some embodiments, the substitution is relative to a human germline heavy chain framework region sequence.

In some embodiments, the anti-TCRβV antibody molecule, e.g., anti-TCRβ V6 (e.g., anti-TCRβ V6-5*01) antibody molecule, comprises a heavy chain variable domain comprising a framework region 3 (FR3) comprising a Threonine at position 73, e.g., a substitution at position 73 according to Kabat numbering, e.g., a Glutamic Acid to Threonine substitution, and a Glycine at position 94, e.g., a substitution at position 94 according to Kabat numbering, e.g., a Arginine to Glycine substitution, e.g., as shown in the amino acid sequence of SEQ ID NO: 10.

In some embodiments, the anti-TCRβV antibody molecule, e.g., anti-TCRβ V6 (e.g., anti-TCRβ V6-5*01) antibody molecule, comprises the heavy chain framework regions 1-4 of A-H.1 or A-H.2, e.g., SEQ ID NO: 9. In some embodiments, the anti-TCRβV antibody molecule, e.g., anti-TCRβ V6 (e.g., anti-TCRβ V6-5*01) antibody molecule, comprises the light chain framework regions 1-4 of A-H.1, e.g., SEQ ID NO: 10. In some embodiments, the anti-TCRβV antibody molecule, e.g., anti-TCRβ V6 (e.g., anti-TCRβ V6-5*01) antibody molecule, comprises the light chain framework regions 1-4 of A-H.2, e.g., SEQ ID NO: 11. In some embodiments, the anti-TCRβV antibody molecule, e.g., anti-TCRβ V6 (e.g., anti-TCRβ V6-5*01) antibody molecule, comprises the heavy chain framework regions 1-4 of A-H.1, e.g., SEQ ID NO: 9; and the light chain framework regions 1-4 of A-H.1, e.g., SEQ ID NO: 10. In some embodiments, the anti-TCRβ 4V antibody molecule, e.g., anti-TCRβ V6 (e.g., anti-TCRβ V6-5*01) antibody molecule, comprises the heavy chain framework regions 1-4 of A-H.2, e.g., SEQ ID NO: 9; and the light chain framework regions 1-4 of A-H.2, e.g., SEQ ID NO: 11.

In some embodiments, the heavy or light chain variable domain, or both, of the anti-TCRβV antibody molecule, e.g., anti-TCRβ V6 (e.g., anti-TCRβ V6-5*01) antibody molecule, includes an amino acid sequence, which is substantially identical to an amino acid as described herein, e.g., at least 80%, 85%, 90%, 92%, 95%, 97%, 98%, 99% or higher identical to a variable region of an antibody described herein, e.g., an antibody chosen from any one of A-H.1 to A-H.85, e.g., A-H.1, A-H.2 or A-H.68, or as described in Table 1, or encoded by the nucleotide sequence in Table 1; or which differs at least 1 or 5 residues, but less than 40, 30, 20, or 10 residues, from a variable region of an antibody described herein.

In some embodiments, the anti-TCRβV antibody molecule, e.g., anti-TCRβ V6 (e.g., anti-TCRβ V6-5*01) antibody molecule, comprises at least one, two, three, or four antigen-binding regions, e.g., variable regions, having an amino acid sequence as set forth in Table 1, or a sequence substantially identical thereto (e.g., a sequence at least about 85%, 90%, 95%, 99% or more identical thereto, or which differs by no more than 1, 2, 5, 10, or 15 amino acid residues from the sequences shown in Table 1. In another embodiment, the anti-TCRβV antibody molecule, e.g., anti-TCRβ V6 (e.g., anti-TCRβ V6-5*01) antibody molecule includes a VH and/or VL domain encoded by a nucleic acid having a nucleotide sequence as set forth in Table 1, or a sequence substantially identical thereto (e.g., a sequence at least about 85%, 90%, 95%, 99% or more identical thereto, or which differs by no more than 3, 6, 15, 30, or 45 nucleotides from the sequences shown in Table 1. 1002141In some embodiments, the anti-TCRβV antibody molecule, e.g., anti-TCRβ V6 (e.g., anti-TCRβ V6-5*01) antibody molecule, comprises: a VH domain comprising the amino acid sequence of SEQ ID NO: 9, an amino acid sequence at least about 85%, 90%, 95%, 99% or more identical to the amino acid sequence of SEQ ID NO: 9, or an amino acid sequence which differs by no more than 1, 2, 5, 10, or 15 amino acid residues from the amino acid sequence of SEQ ID NO: 9; and/or a VL domain comprising the amino acid sequence of SEQ ID NO: 10, an amino acid sequence at least about 85%, 90%, 95%, 99% or more identical to the amino acid sequence of SEQ ID NO: 10, or an amino acid sequence which differs by no more than 1, 2, 5, 10, or 15 amino acid residues from the amino acid sequence of SEQ ID NO: 10.

In some embodiments, the anti-TCRβV antibody molecule, e.g., anti-TCRβ V6 (e.g., anti-TCRβ V6-5*01) antibody molecule, comprises: a VH domain comprising the amino acid sequence of SEQ ID NO: 9, an amino acid sequence at least about 85%, 90%, 95%, 99% or more identical to the amino acid sequence of SEQ ID NO: 9, or an amino acid sequence which differs by no more than 1, 2, 5, 10, or 15 amino acid residues from the amino acid sequence of SEQ ID NO: 9; and/or a VL domain comprising the amino acid sequence of SEQ ID NO: 11, an amino acid sequence at least about 85%, 90%, 95%, 99% or more identical to the amino acid sequence of SEQ ID NO: 11, or an amino acid sequence which differs by no more than 1, 2, 5, 10, or 15 amino acid residues from the amino acid sequence of SEQ ID NO: 11.

In some embodiments, the anti-TCRβV antibody molecule, e.g., anti-TCRβ V6 (e.g., anti-TCRβ V6-5*01) antibody molecule is a full antibody or fragment thereof (e.g., a Fab, F(ab′)₂, Fv, single domain antibody, or a single chain Fv fragment (scFv)). In embodiments, the anti-TCRβV antibody molecule, e.g., anti-TCRβ V6 (e.g., anti-TCRβ V6-5*01) antibody molecule is a monoclonal antibody or an antibody with single specificity. In some embodiments, the anti-TCRβV antibody molecule, e.g., anti-TCRβ V6 (e.g., anti-TCRβ V6-5*01) antibody molecule, can also be a humanized, chimeric, camelid, shark, or an in vitro-generated antibody molecule. In some embodiments, the anti-TCRβV antibody molecule, e.g., anti-TCRβ V6 (e.g., anti-TCRβ V6-5*01) antibody molecule, is a humanized antibody molecule. The heavy and light chains of the anti-TCRβV antibody molecule, e.g., anti-TCRβ V6 (e.g., anti-TCRβ V6-5*01) antibody molecule, can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRβV antibody molecule, e.g., anti-TCRβ V6 (e.g., anti-TCRβ V6-5*01) antibody molecule, is in the form of a multifunctional molecule, e.g., a bispecific molecule, e.g., as described herein.

In some embodiments, the anti-TCRβV antibody molecule, e.g., anti-TCRβ V6 (e.g., anti-TCRβ V6-5*01) antibody molecule, has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1. In some embodiments, the Fc region comprises a Fc region variant, e.g., as described herein.

In some embodiments, the anti-TCRβV antibody molecule, e.g., anti-TCRβ V6 (e.g., anti-TCRβ V6-5*01) antibody molecule, has a light chain constant region chosen from, e.g., the light chain constant regions of kappa or lambda, preferably kappa (e.g., human kappa). In some embodiments, the constant region is altered, e.g., mutated, to modify the properties of the anti-TCRβV antibody molecule, e.g., anti-TCRβ V6 (e.g., anti-TCRβ V6-5*01) antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218), e.g., relative to human IgG1.

Antibody A-H.1 comprises a heavy chain comprising the amino acid sequence of SEQ ID NO: 3278 and a light chain comprising the amino acid sequence of SEQ ID NO: 72. Antibody A-H.2 comprises a heavy chain comprising the amino acid sequence of SEQ ID NO: 3278 and a light chain comprising the amino acid sequence of SEQ ID NO: 3279. Antibody A-H.68 comprises the amino acid sequence of SEQ ID NO: 1337, or a sequence having at least 85%, 90%, 95%, 96/a, 97%, 98%, 99%, 99.5%, 99.9%, or 100% sequence identity thereto. Antibody A-H.69 comprises the amino acid sequence of SEQ ID NO: 1500, or a sequence having at least 85%, 90%, 95%, 96%, 97%, 98%, 99%, 99.5%, 99.9%, or 100% sequence identity thereto.

Additional exemplary humanized anti-TCRβ V6 antibodies are provided in Table 1. In some embodiments, the anti-TCRβ V6 is antibody A. e.g., humanized antibody A (antibody A-H), as provided in Table 1. In some embodiments, the anti-TCRβV antibody comprises one or more (e.g., all three) of a LC CDR1, LC CDR2, and LC CDR3 provided in Table 1; and/or one or more (e.g., all three) of a HC CDR1, HC CDR2, and HC CDR3 provided in Table 1, or a sequence with at least 85%, 90%, 95%, 96%, 97%, 98%, 99%, 99.5%, 99.9%, or 100% sequence identity thereto. In some embodiments, antibody A comprises a variable heavy chain (VH) and/or a variable light chain (VL) provided in Table 1, or a sequence with at least 85%, 90%, 95%, 96%, 97%, 98%, 99%, 99.5%, 99.9%, or 100% sequence identity thereto.

In some embodiments, the anti-TCRβV antibody molecule, e.g., anti-TCRβ V6 (e.g., anti-TCRβ V6-5*01) antibody molecule comprises a VH of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18. A-H.19, A-H.20, A-H.21, A-H.22. A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72. A-H.73, A-H.74, A-H.75, A-H.76, A-H.77. A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VL of A-H.1, A-H.2, A-H.3, A-H.4, A-H.5, A-H.6. A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48. A-H.49, A-H.50, A-H.51, A-H.52, A-H.53. A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VH of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36. A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto; and a VL of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

Exemplary anti-TCRβV antibody molecules and the corresponding TCRβV subfamilies recognized by said anti-TCRβV antibody molecules are disclosed in Table 10A.

The various TCRβV subfamilies and/or subfamily members can be expressed at different levels in individuals. e.g., healthy individuals, as disclosed in Kitaura K. et al (2016), BMC Immunology vol 17: 38, the entire contents of which are hereby incorporated by reference. For example, TCRβ V6-5 is represented in approximately 3-6% healthy donors.

The representation of various TCRBV subfamilies and/or subfamily members can also be different in cancer cells. For example, TCRβV is present in about 3-6% of tumor infiltrating T cells irrespective of tumor type (see Li B. et al., Nature Genetics, 2016, vol:48(7):725-32 the entire contents of which are hereby incorporated by references). Li et al., also disclose that TCRβ V6-5 is present at a high frequency in tumor cells.

### Anti-TCRβ V6 Antibodies

In one aspect, provided herein is an anti-TCRβV antibody molecule that binds to human TCRβ V6, e.g., a TCRβ V6 subfamily comprising: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments the TCRβ V6 subfamily comprises TCRβ V6-5*01 or a variant thereof. In some embodiments, TCRβ V6 comprises TCRβ V6-4*01, or a variant thereof. In some embodiments, TCRβ V6 comprises TCRβ V6-4*02, or a variant thereof. In some embodiments, TCRβ V6 comprises TCRβ V6-9*01, or a variant thereof. In some embodiments, TCRβ V6 comprises TCRβ V6-8*01, or a variant thereof. In some embodiments, TCRβ V6 comprises TCRβ V6-5*01, or a variant thereof. In some embodiments, TCRβ V6 comprises TCRβ V6-6*02, or a variant thereof. In some embodiments, TCRβ V6 comprises TCRβ V6-6*01, or a variant thereof. In some embodiments, TCRβ V6 comprises TCRβ V6-2*01, or a variant thereof. In some embodiments, TCRβ V6 comprises TCRβ V6-3*01, or a variant thereof. In some embodiments, TCRβ V6 comprises TCRβ V6-1*01, or a variant thereof.

In some embodiments, TCRβ V6-5*01 is encoded by the nucleic acid sequence of SEQ ID NO: 43, or a sequence having 85%, 90%, 95%, 99% or more identity thereof. In some embodiments, TCRβ V6-5*01 comprises the amino acid sequence of SEQ ID NO: 44, or an amino acid sequence having 85%, 90%, 95%, 99% or more identity thereof.

In some embodiments, the anti-TCRβV antibody molecule, e.g., anti-TCRβ V6 (e.g., anti-TCRβ V6-5*01) antibody molecule, is a non-murine antibody molecule, e.g., a human or humanized antibody molecule. In some embodiments, the anti-TCRβV antibody molecule, e.g., anti-TCRβ V6 (e.g., anti-TCRβ V6-5*01) antibody molecule is a human antibody molecule. In some embodiments, the anti-TCRβV antibody molecule, e.g., anti-TCRβ V6 (e.g., anti-TCRβ V6-5*01) antibody molecule is a humanized antibody molecule.

In some embodiments, the anti-TCRβV antibody molecule, e.g., anti-TCRβ V6 (e.g., anti-TCRβ V6-5*01) antibody molecule, is isolated or recombinant.

In some embodiments, the anti-TCRβV antibody molecule, e.g., anti-TCRβ V6 (e.g., anti-TCRβ V6-5*01) antibody molecule, comprises at least one antigen-binding region, e.g., a variable region or an antigen-binding fragment thereof, from an antibody described herein. e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one, two, three or four variable regions from an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one or two heavy chain variable regions from an antibody described herein, e.g., an antibody chosen from any one of A-H.1 to A-H.85, e.g., A-H.1, A-H.2 or A-H.68, or an antibody molecule described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one or two light chain variable regions from an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises a heavy chain constant region for an IgG4, e.g., a human IgG4. In still another embodiment, the anti-TCRβV antibody molecule, e.g., anti-TCRβ V6 (e.g., anti-TCRβ V6-5*01) antibody molecule includes a heavy chain constant region for an IgG1, e.g., a human IgG1. In some embodiments, the heavy chain constant region comprises an amino sequence set forth in Table 3, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V6 (e.g., anti-TCRβ V6-5*01) antibody molecule, includes a kappa light chain constant region, e.g., a human kappa light chain constant region. In some embodiments, the light chain constant region comprises an amino sequence set forth in Table 3, or a sequence substantially identical (e.g., at least 80%, 85%, 90%, 92%, 95%, 97/a, 98%, 99% or higher identical) thereto.

In some embodiments, the anti-TCRβV antibody molecule, e.g., anti-TCRβ V6 (e.g., anti-TCRβ V6-5*01) antibody molecule, includes at least one, two, or three complementarity determining regions (CDRs) from a heavy chain variable region (VH) of an antibody described herein. e.g., an antibody chosen from any one of A-H.1 to A-H.85. e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, includes at least one, two, or three CDRs (or collectively all of the CDRs) from a heavy chain variable region comprising an amino acid sequence shown in Table 1, or encoded by a nucleotide sequence shown in Table 1. In some embodiments, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In some embodiments, the anti-TCRβV antibody molecule, e.g., anti-TCRβ V6 (e.g., anti-TCRβ V6-5*01) antibody molecule, includes at least one, two, or three complementarity determining regions (CDRs) from a light chain variable region of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ 4 V6-5*01) antibody molecule, includes at least one, two, or three CDRs (or collectively all of the CDRs) from a light chain variable region comprising an amino acid sequence shown in Table 1, or encoded by a nucleotide sequence shown in Table 1. In some embodiments, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In some embodiments, the anti-TCRβV antibody molecule, e.g., anti-TCRβ V6 (e.g., anti-TCRβ V6-5*01) antibody molecule, includes at least one, two, three, four, five or six CDRs (or collectively all of the CDRs) from a heavy and light chain variable region comprising an amino acid sequence shown in Table 1, or encoded by a nucleotide sequence shown in Table 1. In some embodiments, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In some embodiments, the anti-TCRβV antibody molecule, e.g., anti-TCRβ V6 (e.g., anti-TCRβ V6-5*01) antibody molecule, molecule includes all six CDRs from an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or closely related CDRs, e.g., CDRs which are identical or which have at least one amino acid alteration, but not more than two, three or four alterations (e.g., substitutions, deletions, or insertions, e.g., conservative substitutions). In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule includes at least one, two, or three CDRs according to Kabat et al. (e.g., at least one, two, or three CDRs according to the Kabat definition as set out in Table 1) from a heavy chain variable region of an antibody described herein, e.g., an antibody chosen from any one of A-H.1 to A-H.85, e.g., A-H.1, A-H.2 or A-H.68, or an antibody described in Table 1, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 1.

In some embodiments, the anti-TCRβV antibody molecule, e.g., anti-TCRβ V6 (e.g., anti-TCRβ V6-5*01) antibody molecule includes at least one, two, or three CDRs according to Kabat et al. (e.g., at least one, two, or three CDRs according to the Kabat definition as set out in Table 1) from a light chain variable region of an antibody described herein, e.g., an antibody chosen from any one of A-H.1 to A-H.85, e.g., A-H.1, A-H.2 or A-H.68, or an antibody described in Table 1, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 1.

In some embodiments, the anti-TCRβV antibody molecule, e.g., anti-TCRβ V6 (e.g., anti-TCRβ V6-5*01) antibody molecule, includes at least one, two, three, four, five, or six CDRs according to Kabat et al. (e.g., at least one, two, three, four, five, or six CDRs according to the Kabat definition as set out in Table 1) from the heavy and light chain variable regions of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 1.

In some embodiments, the anti-TCRβV antibody molecule, e.g., anti-TCRβ V6 (e.g., anti-TCRβ V6-5*01) antibody molecule, includes all six CDRs according to Kabat et al. (e.g., all six CDRs according to the Kabat definition as set out in Table 1) from the heavy and light chain variable regions of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1; or a sequence substantially identical (e.g., at least 80%, 85%,o, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 1. In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule, includes at least one, two, or three hypervariable loops that have the same canonical structures as the corresponding hypervariable loop of an antibody described herein, e.g., an antibody chosen from chosen from any one of A-H.1 to A-H.85, e.g., A-H.1, A-H.2 or A-H.68, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In some embodiments, the anti-TCRβV antibody molecule, e.g., anti-TCRβ V6 (e.g., anti-TCRβ V6-5*01) antibody molecule includes at least one, two, or three CDRs according to Chothia et al. (e.g., at least one, two, or three CDRs according to the Chothia definition as set out in Table 1) from a heavy chain variable region of an antibody described herein, e.g., an antibody chosen from any one of A-H.1 to A-H.85, e.g., A-H.1, A-H.2 or A-H.68, or as described in Table 1, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 1.

In some embodiments, the anti-TCRβV antibody molecule, e.g., anti-TCRβ V6 (e.g., anti-TCRβ V6-5*01) antibody molecule includes at least one, two, or three CDRs according to Chothia et al. (e.g., at least one, two, or three CDRs according to the Chothia definition as set out in Table 1) from a light chain variable region of an antibody described herein, e.g., an antibody chosen from any one of A-H.1 to A-H.85, e.g., A-H.1, A-H.2 or A-H.68, or an antibody described in Table 1, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 1.

In some embodiments, the anti-TCRβV antibody molecule, e.g., anti-TCRβ V6 (e.g., anti-TCRβ V6-5*01) antibody molecule, includes at least one, two, three, four, five, or six CDRs according to Chothia er al. (e.g., at least one, two, three, four, five, or six CDRs according to the Chothia definition as set out in Table 1) from the heavy and light chain variable regions of an antibody described herein, e.g., an antibody chosen from any one of A-H.1 to A-H.85, e.g., A-H.1, A-H.2 or A-H.68, or an antibody described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Chothia et al. shown in Table 1.

In some embodiments, the anti-TCRβV antibody molecule, e.g., anti-TCRβ V6 (e.g., anti-TCRβ V6-5*01) antibody molecule, includes all six CDRs according to Chothia et al. (e.g., all six CDRs according to the Chothia definition as set out in Table 1) from the heavy and light chain variable regions of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Chothia et al. shown in Table 1. In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule, molecule includes a combination of CDRs or hypervariable loops defined according to Kabat et al., Chothia et al., or as described in Table 1.

In some embodiments, the anti-TCRβV antibody molecule, e.g., anti-TCRβ V6 (e.g., anti-TCRβ V6-5*01) antibody molecule, can contain any combination of CDRs or hypervariable loops according to the Kabat and Chothia definitions.

In some embodiments, a combined CDR as set out in Table 1 is a CDR that comprises a Kabat CDR and a Chothia CDR. 1002561In some embodiments, the anti-TCRβV antibody molecule, e.g., anti-TCRβ V6 (e.g., anti-TCRβ V6-5*01) antibody molecule, molecule includes a combination of CDRs or hypervariable loops identified as combined CDRs in Table 1. In some embodiments, the anti-TCRβV antibody molecule, e.g., anti-TCRβV6 (e.g., anti-TCRβ V6-5*01) antibody molecule, can contain any combination of CDRs or hypervariable loops according the “combined” CDRs are described in Table 1.

In some embodiments, e.g., an embodiment comprising a variable region, a CDR (e.g., a combined CDR, Chothia CDR or Kabat CDR), or other sequence referred to herein, e.g., in Table 1, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multifunctional molecule, e.g., a bispecific molecule, e.g., as described herein.

In some embodiments, the anti-TCRβV antibody molecule, e.g., anti-TCRβ V6 (e.g., anti-TCRβ V6-5*01) antibody molecule includes: (i) one, two or all of a light chain complementarity determining region 1 (LC CDR1), a light chain complementarity determining region 2 (LC CDR2), and a light chain complementarity determining region 3 (LC CDR3) of SEQ ID NO: 2, SEQ ID NO: 10 or SEQ ID NO: 11, and/or (ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 1 or SEQ ID NO: 9.

In some embodiments, the anti-TCRβV antibody molecule, e.g., anti-TCRβ 4 V6 (e.g., anti-TCRβ V6-5*01) antibody molecule comprises a LC CDR1, LC CDR2, and LC CDR3 of SEQ ID NO: 2, and a HC CDR1, HC CDR2, and HC CDR3 of SEQ ID NO: 1.

In some embodiments the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 10, and a HC CDR1, HC CDR2, and HC CDR3 of SEQ ID NO: 9.

In some embodiments, the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 11, and a HC CDR1, HC CDR2, and HC CDR3 of SEQ ID NO: 9.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 6, a LC CDR2 amino acid sequence of SEQ ID NO: 7, or a LC CDR3 amino acid sequence of SEQ ID NO: 8; and/or (ii) a HC CDR1 amino acid sequence of SEQ ID NO: 3, a HC CDR2 amino acid sequence of SEQ ID NO: 4, or a HC CDR3 amino acid sequence of SEQ ID NO: 5.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 6, a LC CDR2 amino acid sequence of SEQ ID NO: 7, or a LC CDR3 amino acid sequence of SEQ ID NO: 8, and/or (ii) a heavy chain variable region (VH) comprising a HC CDR1 amino acid sequence of SEQ ID NO: 3, a HC CDR2 amino acid sequence of SEQ ID NO: 4, or a HC CDR3 amino acid sequence of SEQ ID NO: 5.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 51, a LC CDR2 amino acid sequence of SEQ ID NO: 52, or a LC CDR3 amino acid sequence of SEQ ID NO: 53; and/or (ii) a HC CDR1 amino acid sequence of SEQ ID NO: 45, a HC CDR2 amino acid sequence of SEQ ID NO: 46, or a HC CDR3 amino acid sequence of SEQ ID NO: 47.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 51, a LC CDR2 amino acid sequence of SEQ ID NO: 52, or a LC CDR3 amino acid sequence of SEQ ID NO: 53; and/or (ii) a heavy chain variable region (VH) comprising a HC CDR1 amino acid sequence of SEQ ID NO: 45, a HC CDR2 amino acid sequence of SEQ ID NO: 46, or a HC CDR3 amino acid sequence of SEQ ID NO: 47.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 54, a LC CDR2 amino acid sequence of SEQ ID NO: 55, or a LC CDR3 amino acid sequence of SEQ ID NO: 56; and/or (ii) a HC CDR1 amino acid sequence of SEQ ID NO: 48, a HC CDR2 amino acid sequence of SEQ ID NO: 49, or a HC CDR3 amino acid sequence of SEQ ID NO: 50.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 54, a LC CDR2 amino acid sequence of SEQ ID NO: 55, or a LC CDR3 amino acid sequence of SEQ ID NO: 56; and/or (ii) a heavy chain variable region (VH) comprising a HC CDR1 amino acid sequence of SEQ ID NO: 48, a HC CDR2 amino acid sequence of SEQ ID NO: 49, or a HC CDR3 amino acid sequence of SEQ ID NO: 50.

In some embodiments, the anti-TCRβV antibody molecule, e.g., anti-TCRβ V6 (e.g., anti-TCRβ V6-5*01) antibody molecule comprises a VH and/or a VL of an antibody described in Table 1, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VH and a VL of an antibody described in Table 1, or a sequence with at least 80%, 85%, 90%, 95%, 96%, 97%, 98%, 99% or more identity thereto.

In some embodiments, an anti-TCRVb antibody as described herein has an antigen binding domain having a VL having a consensus sequence of SEQ ID NO: 230, wherein position 30 is G, E, A or D; position 31 is N or D; position 32 is R or K; position 36 is Y or H; and/or position 56 is K or S.

In some embodiments, an anti-TCRVb antibody as described herein has an antigen binding domain having a VH having a consensus sequence of SEQ ID NO: 231, wherein: position 27 is H or T or G or Y; position 28 is D or T or S; position 30 is H or R or D or K or T; position 31 is L or D or K or T or N; position 32 is W or F or T or I or Y or G; position 49 is R or W; position 50 is V or I or F; position 51 is F or S or Y; position 52 is A or P; position 56 is N or S; position 57 is T or V or Y or I; position 58 is K or R; position 97 is G or V: position 99 is Y or I: position 102 is Y or A; and/or position 103 is D or G.

### Anti-TCRβ V10 Antibodies

In one aspect, provided herein is an anti-TCRβV antibody molecule that binds to a human TCRβ V10 subfamily member. In some embodiments, TCRβ V10 subfamily is also known as TCRβ V12. In some embodiments, the TCRβ V 10 subfamily comprises: TCRβ V10-1*01, TCRβ V10-1*02, TCRβ V10-3*01 or TCRβ V10-2*01, or a variant thereof.

Exemplary anti-TCRβ V10 antibodies are provided in Table 12. In some embodiments, the anti-TCRβ V10 is antibody D, e.g., humanized antibody D (antibody D-H), as provided in Table 12. In some embodiments, antibody D comprises one or more (e.g., three) light chain CDRs and/or one or more (e.g., three) heavy chain CDRs provided in Table 12, or a sequence with at least 95% sequence identity thereto. In some embodiments, antibody D comprises a variable heavy chain (VH) and/or a variable light chain (VL) provided in Table 12, or a sequence with at least 95% sequence identity thereto.

In some embodiments, the anti-TCRβ V10 antibody molecule comprises a VH or a VL of an antibody described in Table 12, or a sequence with at least 80%, 85%, 90%, 95%, 96%, 97%, 98%, 99% or more identity thereto.

In some embodiments, the anti-TCRβ V10 antibody molecule comprises a VH and a VL of an antibody described in Table 12, or a sequence with at least 80%, 85%, 90%, 95%, 96%, 97%, 98%, 99% or more identity thereto.

### Antibody-Like Frameworks or Scaffolds

A wide variety of antibody/immunoglobulin frameworks or scaffolds can be employed in the anti-TCRvb antibody molecules as described herein or multifunctional formats thereof so long as the resulting polypeptide includes at least one binding region which specifically binds to the target antigen, e.g., a TCRvb, a tumor antigen, among others. Such frameworks or scaffolds include the 5 main idiotypes of human immunoglobulins, or fragments thereof, and include immunoglobulins of other animal species, preferably having humanized aspects. Novel frameworks, scaffolds and fragments continue to be discovered and developed by those skilled in the art.

In some embodiments, the anti-TCRvb antibody molecules as described herein or multifunctional formats thereof include non-immunoglobulin based antibodies using non-immunoglobulin scaffolds onto which CDRs can be grafted. Any non-immunoglobulin frameworks and scaffolds may be employed, as long as they comprise a binding region specific for the target antigen (e.g., TCRvb or a tumor antigen). Exemplary non-immunoglobulin frameworks or scaffolds include, but are not limited to, fibronectin (Compound Therapeutics, Inc., Waltham, MA), ankyrin (Molecular Partners AG, Zurich, Switzerland), domain antibodies (Domantis, Ltd., Cambridge, MA, and Ablynx nv, Zwijnaarde, Belgium), lipocalin (Pieris Proteolab AG, Freising, Germany), small modular immuno-pharmaceuticals (Trubion Pharmaceuticals Inc., Seattle, WA), maxybodies (Avidia, Inc., Mountain View, CA), Protein A (Affibody AG, Sweden), and affilin (gamma-crystallin or ubiquitin) (Scil Proteins GmbH, Halle, Germany).

Fibronectin scaffolds are typically based on fibronectin type III domain (e.g., the tenth module of the fibronectin type III (10 Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). Because of this structure, the non-immunoglobulin antibody mimics antigen binding properties that are similar in nature and affinity to those of antibodies. These scaffolds can be used in a loop randomization and shuffling strategy in vitro that is similar to the process of affinity maturation of antibodies in vivo. These fibronectin-based molecules can be used as scaffolds where the loop regions of the molecule can be replaced with CDRs of the invention using standard cloning techniques.

The ankyrin technology is based on using proteins with ankyrin derived repeat modules as scaffolds for bearing variable regions which can be used for binding to different targets. The ankyrin repeat module typically is a about 33 amino acid polypeptide consisting of two anti-parallel α-helices and a β-turn. Binding of the variable regions can be optimized by using ribosome display.

Avimers are used by nature for protein-protein interactions and in human over 250 proteins are structurally based on A-domains. Avimers consist of a number of different “A-domain” monomers (2-10) linked via amino acid linkers. Avimers can be created that can bind to the target antigen using the methodology described in, for example, U.S. Patent Application Publication Nos. 20040175756; 20050053973; 20050048512; and 20060008844.

Affibody affinity ligands are small, simple proteins composed of a three-helix bundle based on the scaffold of one of the IgG-binding domains of Protein A. Protein A is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, they have a molecular weight of 6 kDa, compared to the molecular weight of antibodies, which is 150 kDa. In spite of its small size, the binding site of affibody molecules is similar to that of an antibody.

Anticalins are known commercially, e.g., Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids. The protein architecture is reminiscent of immunoglobulins, with hypervariable loops on top of a rigid framework. However, in contrast with antibodies or their recombinant fragments, lipocalins are composed of a single polypeptide chain with 160 to 180 amino acid residues, being just marginally bigger than a single immunoglobulin domain. The set of four loops, which makes up the binding pocket, shows pronounced structural plasticity and tolerates a variety of side chains. The binding site can thus be reshaped in a proprietary process in order to recognize prescribed target molecules of different shape with high affinity and specificity. One protein of lipocalin family, the bilin-binding protein (BBP) of Pieris Brassicac has been used to develop anticalins by mutagenizing the set of four loops. One example of a patent application describing anticalins is in PCT Publication No. WO 199916873.

Affilin molecules are small non-immunoglobulin proteins which are designed for specific affinities towards proteins and small molecules. New affilin molecules can be very quickly selected from two libraries, each of which is based on a different human derived scaffold protein. Affilin molecules do not show any structural homology to immunoglobulin proteins. Currently, two affilin scaffolds are employed, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

Protein epitope mimetics (PEM) are medium-sized, cyclic, peptide-like molecules (MW 1-2 kDa) mimicking beta-hairpin secondary structures of proteins, the major secondary structure involved in protein-protein interactions.

Domain antibodies (dAbs) can be used in the anti-TCRvb antibody molecules as described herein or multifunctional formats thereof are small functional binding fragments of antibodies, corresponding to the variable regions of either the heavy or light chains of antibodies. Domain antibodies are well expressed in bacterial, yeast, and mammalian cell systems. Further details of domain antibodies and methods of production thereof are known in the art (see, for example, U.S. Pat. Nos. 6,291,158; 6,582,915; 6,593,081; 6,172,197; 6,696,245; European Patents 0368684 & 0616640; WO05/035572, WO04/101790, WO04/081026, WO04/058821, WO04/003019 and WO03/002609. Nanobodies are derived from the heavy chains of an antibody.

A nanobody typically comprises a single variable domain and two constant domains (CH2 and CH3) and retains antigen-binding capacity of the original antibody. Nanobodies can be prepared by methods known in the art (See e.g., U.S. Pat. No. 6,765,087. U.S. Pat. No. 6,838,254, WO 06/079372). Unibodies consist of one light chain and one heavy chain of an IgG4 antibody. Unibodies may be made by the removal of the hinge region of IgG4 antibodies. Further details of unibodies and methods of preparing them may be found in WO2007/059782.

### Anti-TCRβVP Antibody Effector Function and Fc Variants

In some embodiments, an anti-TCRVβ antibody as described herein comprises an Fc region, e.g., as described herein. In some embodiments, the Fc region is a wildtype Fc region, e.g., a wildtype human Fc region. In some embodiments, the Fc region comprises a variant, e.g., an Fc region comprising an addition, substitution, or deletion of at least one amino acid residue in the Fc region which results in, e.g., reduced or ablated affinity for at least one Fc receptor.

The Fc region of an antibody interacts with a number of receptors or ligands including Fc Receptors (e.g., FcγRI, FcγRIIA, FcγRIIIA), the complement protein CIq, and other molecules such as proteins A and G. These interactions are essential for a variety of effector functions and downstream signaling events including: antibody dependent cell-mediated cytotoxicity (ADCC), Antibody-dependent cellular phagocytosis (ADCP) and complement dependent cytotoxicity (CDC).

In some embodiments, an anti-TCRVβ antibody comprising a variant Fc region has reduced, e.g., ablated, affinity for an Fc receptor, e.g., an Fc receptor described herein. In some embodiments, the reduced affinity is compared to an otherwise similar antibody with a wildtype Fc region.

In some embodiments, an anti-TCRVβ antibody comprising a variant Fc region has one or more of the following properties: (1) reduced effector function (e.g., reduced ADCC, ADCP and/or CDC); (2) reduced binding to one or more Fc receptors; and/or (3) reduced binding to C1q complement. In some embodiments, the reduction in any one, or all of properties (1)-(3) is compared to an otherwise similar antibody with a wildtype Fc region.

In some embodiments, an anti-TCRVβ antibody comprising a variant Fc region has reduced affinity to a human Fc receptor, e.g., FcγR I, FcγR II and/or FcγR III. In some embodiments, the anti-TCRVβ antibody comprising a variant Fc region comprises a human IgG1 region or a human IgG4 region.

In some embodiments, an anti-TCRVβ antibody comprising a variant Fc region activates and/or expands T cells, e.g., as described herein. In some embodiments, an anti-TCRVβ antibody comprising a variant Fc region has a cytokine profile described herein, e.g., a cytokine profile that differs from a cytokine profile of a T cell engager that binds to a receptor or molecule other than a TCRβV region (“a non-TCRβV-binding T cell engager”). In some embodiments, the non-TCRβV-binding T cell engager comprises an antibody that binds to a CD3 molecule (e.g., CD3 epsilon (CD3e) molecule): or a TCR alpha (TCRα) molecule.

Exemplary Fc region variants are provided in Table 14 and also disclosed in Saunders O, (2019) Frontiers in Immunology; vol 10, article1296, the entire contents of which is hereby incorporated by reference.

In some embodiments, an anti-TCRVβ antibody as described herein comprises any one or all, or any combination of Fc region variants disclosed in Table 14.

In some embodiments, an anti-TCRVβ antibody as described herein comprises any one or all, or any combination of Fc region variants. e.g., mutations, disclosed in Table 14. In some embodiments, an anti-TCRVβ antibody as described herein comprise an Asn297Ala (N297A) mutation. In some embodiments, an anti-TCRVβ antibody as described herein comprise a Leu234Ala/Leu235Ala (LALA) mutation.

### Multifunctional Molecules

The terms “multifunctional molecule” and “multispecific molecule.” as used herein interchangeably, refer to a molecule, e.g., a polypeptide, that has two or more functionalities, e.g., two or more binding specificities. In some embodiments, the functionalities can include one or more immune cell engagers, one or more tumor binding molecules, one or more cytokine molecules, one or more stromal modifiers, and other moieties described herein. In some embodiments, the multifunctional molecule is a multifunctional antibody molecule, e.g., a bispecific antibody molecule. In some embodiments, the multifunctional molecule includes an anti-TCRVb antibody molecule as described herein.

Described herein, in certain embodiments, is a multifunctional polypeptide molecule comprising a first polypeptide, a second polypeptide, a third polypeptide, a fourth polypeptide, and at least one cytokine polypeptide or a functional fragment or a functional variant thereof, wherein the first polypeptide, the second polypeptide, the third polypeptide, and the fourth polypeptide are non-contiguous, wherein: (i) the first polypeptide comprising a first portion of a first T cell receptor variable beta (TCRβV)-binding moiety and a first dimerization module linked to the first portion of the first TCRβV-binding moiety; (ii) the second polypeptide comprising a second portion of the first TCRβV-binding moiety; (iii) the third polypeptide comprising a first portion of a second TCRβV-binding moiety and a second dimerization module linked to the first portion of the second TCRβV-binding moiety; and (iv) the fourth polypeptide comprising a second portion of the second TCRβV-binding moiety; and wherein the at least one cytokine polypeptide or a functional fragment or a functional variant thereof is covalently linked to the first polypeptide, the second polypeptide, the third polypeptide, the fourth polypeptide, or a combination thereof.

Described herein, in certain embodiments, is a multifunctional polypeptide molecule comprising a first polypeptide, a second polypeptide, a third polypeptide, and at least one cytokine polypeptide or a functional fragment or a functional variant thereof, wherein the first polypeptide, the second polypeptide, and the third polypeptide are non-contiguous, wherein: (i) the first polypeptide comprising a first portion of a first TCRβV-binding moiety and a first dimerization module linked to the first portion of the first TCRβV-binding moiety; (ii) the second polypeptide comprising a second portion of the first TCRβV-binding moiety; and (iii) the third polypeptide comprising a second dimerization module; and wherein the at least one cytokine polypeptide or a functional fragment or a functional variant thereof is covalently linked to the first polypeptide, the second polypeptide, the third polypeptide, or a combination thereof.

Described herein, in certain embodiments, is a multifunctional polypeptide molecule comprising a first polypeptide, a second polypeptide, a third polypeptide, and at least one cytokine polypeptide or a functional fragment or a functional variant thereof, wherein the first polypeptide, the second polypeptide, and the third polypeptide are non-contiguous, wherein: (i) the first polypeptide comprising a first portion of a first TCRβV-binding moiety and a first dimerization module linked to the first portion of the first TCRβV-binding moiety; (ii) the second polypeptide comprising a second portion of the first TCRβV-binding moiety; and (iii) the third polypeptide comprising a second dimerization module: wherein the at least one cytokine polypeptide or a functional fragment or a functional variant thereof is covalently linked to the first polypeptide, the second polypeptide, the third polypeptide, or a combination thereof; and wherein the multifunctional polypeptide molecule does not comprise an additional TCRβV-binding moiety except the first TCRβV-binding moiety.

In some embodiments, the first portion of the first TCRβV-binding moiety comprises a first heavy chain variable domain (VH) and a first heavy chain constant domain 1 (CH1) linked to the first VH. In some embodiments, the first CH1 is linked to the C-terminus of the first VH. In some embodiments, the second portion of the first TCRβV-binding moiety comprises a first light chain variable domain (VL) and a first light chain constant domain (CL) linked to the first VL. In some embodiments, first CL is linked to the C-terminus of the first VL. In some embodiments, wherein the first dimerization module is linked to the first portion of the first TCRβV-binding moiety. In some embodiments, the first dimerization module is linked to the C-terminus of the first portion of the first TCRβV-binding moiety. In some embodiments, wherein the first portion of the second TCRβV-binding moiety comprises a second VH and a second CH1 linked to the second VH. In some embodiments, the second CH1 is linked to the C-terminus of the second VH. In some embodiments, the second portion of the second TCRβV-binding moiety comprises a second VL and a second CL linked to the second VL. In some embodiments, the second CL is linked to the C-terminus of the second VL. In some embodiments, the second dimerization module is linked to the first portion of the second TCRβV-binding moiety. In some embodiments, the second dimerization module is linked to the C-terminus of the first portion of the second TCRβV-binding moiety.

In some embodiments, (a) the N-terminus of the first polypeptide is linked to a first cytokine polypeptide or a functional fragment or a functional variant thereof: the C-terminus of the first polypeptide is linked to a second cytokine polypeptide or a functional fragment or a functional variant thereof; or a combination thereof; (b) the N-terminus of the second polypeptide is linked to a third cytokine polypeptide or a functional fragment or a functional variant thereof; the C-terminus of the second polypeptide is linked to a fourth cytokine polypeptide or a functional fragment or a functional variant thereof; or a combination thereof, (c) the N-terminus of the third polypeptide is linked to a fifth cytokine polypeptide or a functional fragment or a functional variant thereof: the C-terminus of the third polypeptide is linked to a sixth cytokine polypeptide or a functional fragment or a functional variant thereof; or a combination thereof: (d) the N-terminus of the fourth polypeptide is linked to a seventh cytokine polypeptide or a functional fragment or a functional variant thereof, the C-terminus of the fourth polypeptide is linked to an eighth cytokine polypeptide or a functional fragment or a functional variant thereof: or a combination thereof; or (e) a combination thereof.

In some embodiments, (a-1) the N-terminus of the first polypeptide is linked to the first cytokine polypeptide or a functional fragment or a functional variant thereof; the C-terminus of the first polypeptide is linked to the second cytokine polypeptide or a functional fragment or a functional variant thereof; or a combination thereof; and (a-2) the N-terminus of the second polypeptide is linked to the third cytokine polypeptide or a functional fragment or a functional variant thereof; the C-terminus of the second polypeptide is linked to the fourth cytokine polypeptide or a functional fragment or a functional variant thereof, or a combination thereof; (b-1) the N-terminus of the first polypeptide is linked to the first cytokine polypeptide or a functional fragment or a functional variant thereof; the C-terminus of the first polypeptide is linked to the second cytokine polypeptide or a functional fragment or a functional variant thereof; or a combination thereof; and (b-2) the N-terminus of the third polypeptide is linked to the fifth cytokine polypeptide or a functional fragment or a functional variant thereof; the C-terminus of the third polypeptide is linked to the sixth cytokine polypeptide or a functional fragment or a functional variant thereof; or a combination thereof; (c-1) the N-terminus of the first polypeptide is linked to the first cytokine polypeptide or a functional fragment or a functional variant thereof; the C-terminus of the first polypeptide is linked to the second cytokine polypeptide or a functional fragment or a functional variant thereof; or a combination thereof; and (c-2) the N-terminus of the fourth polypeptide is linked to the seventh cytokine polypeptide or a functional fragment or a functional variant thereof; the C-terminus of the fourth polypeptide is linked to the eighth cytokine polypeptide or a functional fragment or a functional variant thereof; or a combination thereof; (d-1) the N-terminus of the second polypeptide is linked to the third cytokine polypeptide or a functional fragment or a functional variant thereof: the C-terminus of the second polypeptide is linked to the fourth cytokine polypeptide or a functional fragment or a functional variant thereof; or a combination thereof; and (d-2) the N-terminus of the third polypeptide is linked to the fifth cytokine polypeptide or a functional fragment or a functional variant thereof: the C-terminus of the third polypeptide is linked to the sixth cytokine polypeptide or a functional fragment or a functional variant thereof; or a combination thereof; (e-1) the N-terminus of the second polypeptide is linked to the third cytokine polypeptide or a functional fragment or a functional variant thereof; the C-terminus of the second polypeptide is linked to the fourth cytokine polypeptide or a functional fragment or a functional variant thereof; or a combination thereof; and (c-2) the N-terminus of the fourth polypeptide is linked to the seventh cytokine polypeptide or a functional fragment or a functional variant thereof: the C-terminus of the fourth polypeptide is linked to the eighth cytokine polypeptide or a functional fragment or a functional variant thereof; or a combination thereof; or (f-1) the N-terminus of the third polypeptide is linked to the fifth cytokine polypeptide or a functional fragment or a functional variant thereof: the C-terminus of the third polypeptide is linked to the sixth cytokine polypeptide or a functional fragment or a functional variant thereof; or a combination thereof; and (f-2) the N-terminus of the fourth polypeptide is linked to the seventh cytokine polypeptide or a functional fragment or a functional variant thereof; the C-terminus of the fourth polypeptide is linked to the eighth cytokine polypeptide or a functional fragment or a functional variant thereof: or a combination thereof.

In some embodiments, (a-1) the N-terminus of the first polypeptide is linked to the first cytokine polypeptide or a functional fragment or a functional variant thereof; the C-terminus of the first polypeptide is linked to the second cytokine polypeptide or a functional fragment or a functional variant thereof; or a combination thereof; (a-2) the N-terminus of the second polypeptide is linked to the third cytokine polypeptide or a functional fragment or a functional variant thereof; the C-terminus of the second polypeptide is linked to the fourth cytokine polypeptide or a functional fragment or a functional variant thereof; or a combination thereof; and (a-3) the N-terminus of the third polypeptide is linked to the fifth cytokine polypeptide or a functional fragment or a functional variant thereof; the C-terminus of the third polypeptide is linked to the sixth cytokine polypeptide or a functional fragment or a functional variant thereof, or a combination thereof; (b-1) the N-terminus of the first polypeptide is linked to the first cytokine polypeptide or a functional fragment or a functional variant thereof; the C-terminus of the first polypeptide is linked to the second cytokine polypeptide or a functional fragment or a functional variant thereof; or a combination thereof; (b-2) the N-terminus of the second polypeptide is linked to the third cytokine polypeptide or a functional fragment or a functional variant thereof; the C-terminus of the second polypeptide is linked to the fourth cytokine polypeptide or a functional fragment or a functional variant thereof; or a combination thereof; and (b-3) the N-terminus of the fourth polypeptide is linked to the seventh cytokine polypeptide or a functional fragment or a functional variant thereof; the C-terminus of the fourth polypeptide is linked to the eighth cytokine polypeptide or a functional fragment or a functional variant thereof; or a combination thereof; or (c-1) the N-terminus of the second polypeptide is linked to the third cytokine polypeptide or a functional fragment or a functional variant thereof; the C-terminus of the second polypeptide is linked to the fourth cytokine polypeptide or a functional fragment or a functional variant thereof; or a combination thereof; (c-2) the N-terminus of the third polypeptide is linked to the fifth cytokine polypeptide or a functional fragment or a functional variant thereof; the C-terminus of the third polypeptide is linked to the sixth cytokine polypeptide or a functional fragment or a functional variant thereof; or a combination thereof; and (c-3) the N-terminus of the fourth polypeptide is linked to the seventh cytokine polypeptide or a functional fragment or a functional variant thereof; the C-terminus of the fourth polypeptide is linked to the eighth cytokine polypeptide or a functional fragment or a functional variant thereof; or a combination thereof.

In some embodiments. (1) the N-terminus of the first polypeptide is linked to the first cytokine polypeptide or a functional fragment or a functional variant thereof; the C-terminus of the first polypeptide is linked to the second cytokine polypeptide or a functional fragment or a functional variant thereof; or a combination thereof; (2) the N-terminus of the second polypeptide is linked to the third cytokine polypeptide or a functional fragment or a functional variant thereof; the C-terminus of the second polypeptide is linked to the fourth cytokine polypeptide or a functional fragment or a functional variant thereof; or a combination thereof; (3) the N-terminus of the third polypeptide is linked to the fifth cytokine polypeptide or a functional fragment or a functional variant thereof; the C-terminus of the third polypeptide is linked to the sixth cytokine polypeptide or a functional fragment or a functional variant thereof; or a combination thereof; and (4) the N-terminus of the fourth polypeptide is linked to the seventh cytokine polypeptide or a functional fragment or a functional variant thereof; the C-terminus of the fourth polypeptide is linked to the eighth cytokine polypeptide or a functional fragment or a functional variant thereof; or a combination thereof.

In some embodiments, the first cytokine polypeptide, the second cytokine polypeptide, or a combination thereof is within a single contiguous polypeptide chain of the first polypeptide, the third cytokine polypeptide, the fourth cytokine polypeptide, or a combination thereof is within a single contiguous polypeptide chain of the second polypeptide, the fifth cytokine polypeptide, the sixth cytokine polypeptide, or a combination thereof is within a single contiguous polypeptide chain of the third polypeptide, the seventh cytokine polypeptide, the eighth cytokine polypeptide, or a combination thereof is within a single contiguous polypeptide chain of the fourth polypeptide, or a combination thereof.

In some embodiments, (a) the N-terminus of the first polypeptide is linked to a first cytokine polypeptide or a functional fragment or a functional variant thereof; the C-terminus of the first polypeptide is linked to a second cytokine polypeptide or a functional fragment or a functional variant thereof; or a combination thereof; (b) the N-terminus of the second polypeptide is linked to a third cytokine polypeptide or a functional fragment or a functional variant thereof; the C-terminus of the second polypeptide is linked to a fourth cytokine polypeptide or a functional fragment or a functional variant thereof; or a combination thereof; (c) the N-terminus of the third polypeptide is linked to a fifth cytokine polypeptide or a functional fragment or a functional variant thereof; the C-terminus of the third polypeptide is linked to a sixth cytokine polypeptide or a functional fragment or a functional variant thereof; or a combination thereof; or (d) a combination thereof.

In some embodiments, (a-1) the N-terminus of the first polypeptide is linked to the first cytokine polypeptide or a functional fragment or a functional variant thereof; the C-terminus of the first polypeptide is linked to the second cytokine polypeptide or a functional fragment or a functional variant thereof; or a combination thereof; and (a-2) the N-terminus of the second polypeptide is linked to the third cytokine polypeptide or a functional fragment or a functional variant thereof; the C-terminus of the second polypeptide is linked to the fourth cytokine polypeptide or a functional fragment or a functional variant thereof; or a combination thereof; (b-1) the N-terminus of the first polypeptide is linked to the first cytokine polypeptide or a functional fragment or a functional variant thereof; the C-terminus of the first polypeptide is linked to the second cytokine polypeptide or a functional fragment or a functional variant thereof; or a combination thereof; and (b-2) the N-terminus of the third polypeptide is linked to the fifth cytokine polypeptide or a functional fragment or a functional variant thereof; the C-terminus of the third polypeptide is linked to the sixth cytokine polypeptide or a functional fragment or a functional variant thereof; or a combination thereof; or (c-1) the N-terminus of the second polypeptide is linked to the third cytokine polypeptide or a functional fragment or a functional variant thereof; the C-terminus of the second polypeptide is linked to the fourth cytokine polypeptide or a functional fragment or a functional variant thereof: or a combination thereof; and (c-2) the N-terminus of the third polypeptide is linked to the fifth cytokine polypeptide or a functional fragment or a functional variant thereof; the C-terminus of the third polypeptide is linked to the sixth cytokine polypeptide or a functional fragment or a functional variant thereof; or a combination thereof.

In some embodiments, (1) the N-terminus of the first polypeptide is linked to the first cytokine polypeptide or a functional fragment or a functional variant thereof: the C-terminus of the first polypeptide is linked to the second cytokine polypeptide or a functional fragment or a functional variant thereof; or a combination thereof; (2) the N-terminus of the second polypeptide is linked to the third cytokine polypeptide or a functional fragment or a functional variant thereof; the C-terminus of the second polypeptide is linked to the fourth cytokine polypeptide or a functional fragment or a functional variant thereof, or a combination thereof; and (3) the N-terminus of the third polypeptide is linked to the fifth cytokine polypeptide or a functional fragment or a functional variant thereof; the C-terminus of the third polypeptide is linked to the sixth cytokine polypeptide or a functional fragment or a functional variant thereof: or a combination thereof.

In some embodiments, the first cytokine polypeptide, the second cytokine polypeptide, or a combination thereof is within a single contiguous polypeptide chain of the first polypeptide, the third cytokine polypeptide, the fourth cytokine polypeptide, or a combination thereof is within a single contiguous polypeptide chain of the second polypeptide, the fifth cytokine polypeptide, the sixth cytokine polypeptide, or a combination thereof is within a single contiguous polypeptide chain of the third polypeptide, or a combination thereof.

In some embodiments, the multifunctional polypeptide molecule as described herein further comprises a linker between the first portion of the first TCRβV-binding moiety and the first dimerization module, a linker between the first portion of the second TCRβV-binding moiety and the second dimerization module, a linker between the first VH and the first CH1, a linker between the first VL and the first CL, a linker between the second VH and the second CH1, a linker between the second VL and the second CL, a linker between the at least one cytokine polypeptide or a functional fragment or a functional variant thereof and the first polypeptide, a linker between the at least one cytokine polypeptide or a functional fragment or a functional variant thereof and the second polypeptide, a linker between the at least one cytokine polypeptide or a functional fragment or a functional variant thereof and the third polypeptide, a linker between the at least one cytokine polypeptide or a functional fragment or a functional variant thereof and the fourth polypeptide, or a combination thereof.

In some embodiments, the multifunctional polypeptide molecule as described herein further comprises comprising a linker between the first portion of the first TCRβV-binding moiety and the first dimerization module, a linker between the first VH and the first CH1, a linker between the first VL and the first CL, a linker between the at least one cytokine polypeptide or a functional fragment or a functional variant thereof and the first polypeptide, a linker between the at least one cytokine polypeptide or a functional fragment or a functional variant thereof and the second polypeptide, a linker between the at least one cytokine polypeptide or a functional fragment or a functional variant thereof and the third polypeptide, or a combination thereof. In some embodiments, linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

Described herein, in certain embodiments, is a multifunctional polypeptide molecule comprising a first polypeptide, a second polypeptide, a third polypeptide, a fourth polypeptide, a first cytokine polypeptide or a functional fragment or a functional variant thereof, and a second cytokine polypeptide or a functional fragment or a functional variant thereof, wherein the first polypeptide, the second polypeptide, the third polypeptide, and the fourth polypeptide are non-contiguous, wherein: (i) the first polypeptide comprising a first portion of a first TCRβV-binding moiety and a first dimerization module linked to the first portion of the first TCRβV-binding moiety; (ii) the second polypeptide comprising a second portion of the first TCRβV-binding moiety; (iii) the third polypeptide comprising a first portion of a second TCRβV-binding moiety and a second dimerization module linked to the first portion of the second TCRβV-binding moiety; and (iv) the fourth polypeptide comprising a second portion of the second TCRβV-binding moiety; and wherein the first cytokine polypeptide or a functional fragment or a functional variant thereof is covalently linked to the C-terminus of the second polypeptide, and the second cytokine polypeptide or a functional fragment or a functional variant thereof is covalently linked to the C-terminus of the fourth polypeptide.

Described herein, in certain embodiments, is a multifunctional polypeptide molecule comprising a first polypeptide, a second polypeptide, a third polypeptide, a fourth polypeptide, a cytokine polypeptide or a functional fragment or a functional variant thereof, wherein the first polypeptide, the second polypeptide, the third polypeptide, and the fourth polypeptide are non-contiguous, wherein: (i) the first polypeptide comprising a first portion of a first TCRβV-binding moiety and a first dimerization module linked to the first portion of the first TCRβV-binding moiety; (ii) the second polypeptide comprising a second portion of the first TCRβV-binding moiety: (iii) the third polypeptide comprising a first portion of a second TCRβV-binding moiety and a second dimerization module linked to the first portion of the second TCRβV-binding moiety; and (iv) the fourth polypeptide comprising a second portion of the second TCRβV-binding moiety; and wherein the cytokine polypeptide or a functional fragment or a functional variant thereof is covalently linked to the C-terminus of the second polypeptide or the C-terminus of the fourth polypeptide.

Described herein, in certain embodiments, is a multifunctional polypeptide molecule comprising a first polypeptide, a second polypeptide, a third polypeptide, a fourth polypeptide, a cytokine polypeptide or a functional fragment or a functional variant thereof, wherein the first polypeptide, the second polypeptide, the third polypeptide, and the fourth polypeptide are non-contiguous, wherein: (i) the first polypeptide comprising a first portion of a first TCRβV-binding moiety and a first dimerization module linked to the first portion of the first TCRβV-binding moiety; (ii) the second polypeptide comprising a second portion of the first TCRβV-binding moiety; (iii) the third polypeptide comprising a first portion of a second TCRβV-binding moiety and a second dimerization module linked to the first portion of the second TCRβV-binding moiety; and (iv) the fourth polypeptide comprising a second portion of the second TCRβV-binding moiety; and wherein the cytokine polypeptide or a functional fragment or a functional variant thereof is covalently linked to the C-terminus of the first polypeptide or the C-terminus of the third polypeptide.

Described herein, in certain embodiments, is a multifunctional polypeptide molecule comprising a first polypeptide, a second polypeptide, a third polypeptide, and a cytokine polypeptide or a functional fragment or a functional variant thereof, wherein the first polypeptide, the second polypeptide, and the third polypeptide are non-contiguous, wherein: (i) the first polypeptide comprising a first portion of a first TCRβV-binding moiety and a first dimerization module linked to the first portion of the first TCRβV-binding moiety; (ii) the second polypeptide comprising a second portion of the first TCRβV-binding moiety; and (iii) the third polypeptide comprising a second dimerization module; wherein the at least one cytokine polypeptide or a functional fragment or a functional variant thereof is covalently linked to the N terminus of the third polypeptide; and wherein the multifunctional polypeptide molecule does not comprise an additional TCR 3V-binding moiety except the first TCRβV-binding moiety.

In some embodiments, the first portion of the first TCRβV-binding moiety comprises a first VH and a first CH1 linked to the first VH. In some embodiments, the first CH1 is linked to the C-terminus of the first VH.

In some embodiments, the second portion of the first TCRβV-binding moiety comprises a first VL and a first CL linked to the first VL. In some embodiments, first CL is linked to the C-terminus of the first VL.

In some embodiments, the first dimerization module is linked to the first portion of the first TCRβV-binding moiety. In some embodiments, the first dimerization module is linked to the C-terminus of the first portion of the first TCRβV-binding moiety. In some embodiments, the first portion of the second TCRβV-binding moiety comprises a second VH and a second CH1 linked to the second VH. In some embodiments, the second CH1 is linked to the C-terminus of the second VH. In some embodiments, the second portion of the second TCRβV-binding moiety comprises a second VL and a second CL linked to the second VL. In some embodiments, the second CL is linked to the C-terminus of the second VL. In some embodiments, the second dimerization module is linked to the first portion of the second TCRβV-binding moiety. In some embodiments, the second dimerization module is linked to the C-terminus of the first portion of the second TCRβV-binding moiety.

In some embodiments, the multifunctional polypeptide molecule as described herein further comprises a linker between the first portion of the first TCRβV-binding moiety and the first dimerization module, a linker between the first portion of the second TCRβV-binding moiety and the second dimerization module, a linker between the first VH and the first CH1, a linker between the first VL and the first CL, a linker between the second VH and the second CH1, a linker between the second VL and the second CL, a linker between the at least one cytokine polypeptide or a functional fragment or a functional variant thereof and the first polypeptide, a linker between the at least one cytokine polypeptide or a functional fragment or a functional variant thereof and the second polypeptide, a linker between the at least one cytokine polypeptide or a functional fragment or a functional variant thereof and the third polypeptide, a linker between the at least one cytokine polypeptide or a functional fragment or a functional variant thereof and the fourth polypeptide, or a combination thereof. In some embodiments, the multifunctional polypeptide molecule as described herein further comprises a linker between the first portion of the first TCRβV-binding moiety and the first dimerization module, a linker between the first VH and the first CH1, a linker between the first VL and the first CL, a linker between the at least one cytokine polypeptide or a functional fragment or a functional variant thereof and the third polypeptide, or a combination thereof. In some embodiments, linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

In some embodiments, the first TCRβV-binding moiety, the second TCRβV-binding moiety, or a combination thereof comprises any one selected from the group consisting of a Fab, F(ab′)2, Fv, a single chain Fv (scFv), a single domain antibody, a diabody (dAb), a camelid antibody and a combination thereof. In some embodiments, the first TCRβV-binding moiety, the second TCRβV-binding moiety, or a combination thereof comprises a scFv or a Fab.

In some embodiments, the multifunctional polypeptide molecule does not comprise an additional antigen-binding moiety except the TCRβV-binding moiety. In some embodiments, the multifunctional polypeptide molecule further comprise an additional antigen-binding moiety that is not the TCRβV-binding moiety.

Described herein, in certain embodiments, is a multifunctional polypeptide molecule comprising a first polypeptide, a second polypeptide, and at least one cytokine polypeptide or a functional fragment or a functional variant thereof, wherein the first polypeptide and the second polypeptide are non-contiguous, wherein: (i) the first polypeptide comprising a first TCRβV-binding moiety and a first dimerization module linked to the C-terminus of the first TCRβV-binding moiety, wherein the first TCRβV-binding moiety comprises a first VL and a first VH; and (ii) the second polypeptide comprising a second TCRβV-binding moiety and a second dimerization module linked to the C-terminus of the second TCRβV-binding moiety; wherein the at least one cytokine polypeptide or a functional fragment or a functional variant thereof is covalently linked to the first polypeptide, the second polypeptide, or a combination thereof; wherein the first TCRβV-binding moiety, the second TCRβV-binding moiety, or a combination thereof comprises a scFv; and wherein the multifunctional polypeptide molecule does not comprise an additional antigen-binding moiety except the first TCRβV-binding moiety and the second TCRβV-binding moiety.

Described herein, in certain embodiments, is a multifunctional polypeptide molecule comprising a first polypeptide, a second polypeptide, and at least one cytokine polypeptide or a functional fragment or a functional variant thereof, wherein the first polypeptide and the second polypeptide are non-contiguous, wherein: (i) the first polypeptide comprising a first TCRβV-binding moiety and a first dimerization module linked to the C-terminus of the first TCRβV-binding moiety, wherein the first TCRβV-binding moiety comprises a first VL and a first VH; and (ii) the second polypeptide comprising a second dimerization module; wherein the at least one cytokine polypeptide or a functional fragment or a functional variant thereof is covalently linked to the first polypeptide, the second polypeptide, or a combination thereof; wherein the first TCRβV-binding moiety comprises a scFv; wherein the multifunctional polypeptide molecule does not comprise an additional antigen-binding moiety except the first TCRβV-binding moiety; and wherein the multifunctional polypeptide molecule does not comprise an additional TCRβV-binding moiety except the first TCRβV-binding moiety.

In some embodiments, (a) the N-terminus of the first polypeptide is linked to a first cytokine polypeptide or a functional fragment or a functional variant thereof; the C-terminus of the first polypeptide is linked to a second cytokine polypeptide or a functional fragment or a functional variant thereof; or a combination thereof; (b) the N-terminus of the second polypeptide is linked to a third cytokine polypeptide or a functional fragment or a functional variant thereof; the C-terminus of the second polypeptide is linked to a fourth cytokine polypeptide or a functional fragment or a functional variant thereof; or a combination thereof; or (e) a combination thereof.

In some embodiments, the first cytokine polypeptide, the second cytokine polypeptide, or a combination thereof is within a single contiguous polypeptide chain of the first polypeptide, the third cytokine polypeptide, the fourth cytokine polypeptide, or a combination thereof is within a single contiguous polypeptide chain of the second polypeptide, or a combination thereof.

In some embodiments, the multifunctional polypeptide molecule as described herein further comprises a linker between the first TCRβV-binding moiety and the first dimerization module, a linker between the second TCRβV-binding moiety and the second dimerization module, a linker between the at least one cytokine polypeptide or a functional fragment or a functional variant thereof and the first polypeptide, a linker between the at least one cytokine polypeptide or a functional fragment or a functional variant thereof and the second polypeptide, or a combination thereof.

In some embodiments, the multifunctional polypeptide molecule as described herein further comprises a linker between the first TCRβV-binding moiety and the first dimerization module, a linker between the at least one cytokine polypeptide or a functional fragment or a functional variant thereof and the first polypeptide, a linker between the at least one cytokine polypeptide or a functional fragment or a functional variant thereof and the second polypeptide, or a combination thereof. In some embodiments, the linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

In some embodiments, the multifunctional polypeptide molecule comprises at least two of the cytokine polypeptide. In some embodiments, the multifunctional polypeptide molecule comprises at least three of the cytokine polypeptide. In some embodiments, the multifunctional polypeptide molecule comprises at least four of the cytokine polypeptide. In some embodiments, the multifunctional polypeptide molecule comprises at least five of the cytokine polypeptide. In some embodiments, the multifunctional polypeptide molecule comprises at least six of the cytokine polypeptide. In some embodiments, the multifunctional polypeptide molecule comprises at least seven of the cytokine polypeptide. In some embodiments, the multifunctional polypeptide molecule comprises at least eight of the cytokine polypeptide. In some embodiments, the multifunctional polypeptide molecule comprises two of the cytokine polypeptide. In some embodiments, the multifunctional polypeptide molecule comprises three of the cytokine polypeptide. In some embodiments, the multifunctional polypeptide molecule comprises four of the cytokine polypeptide. In some embodiments, the multifunctional polypeptide molecule comprises five of the cytokine polypeptide. In some embodiments, the multifunctional polypeptide molecule comprises six of the cytokine polypeptide. In some embodiments, the multifunctional polypeptide molecule comprises seven of the cytokine polypeptide. In some embodiments, the multifunctional polypeptide molecule comprises eight of the cytokine polypeptide. In some embodiments, the multifunctional polypeptide molecule comprises two of the cytokine polypeptide, each of which is linked to the first polypeptide and the second polypeptide; the first polypeptide and the third polypeptide, the first polypeptide and the fourth polypeptide: the second and the third polypeptide: the second polypeptide and the fourth polypeptide; or the third polypeptide and the fourth polypeptide, respectively. In some embodiments, the multifunctional polypeptide molecule comprises three of the cytokine polypeptide, each of which is linked to the first polypeptide, the second polypeptide, and the third polypeptide: the first polypeptide, the second polypeptide, and the fourth polypeptide; the first polypeptide, the third polypeptide, and the fourth polypeptide; or the second polypeptide, the third polypeptide, and the fourth polypeptide, respectively. In some embodiments, the multifunctional polypeptide molecule comprises four of the cytokine polypeptide, each of which is linked to the first polypeptide, the second polypeptide, the third polypeptide, and the fourth polypeptide, respectively. In some embodiments, the cytokine polypeptide is not linked to the polypeptides that comprise the first TCRβV-binding moiety.

In some embodiments, the at least one cytokine polypeptide is selected from the group consisting of interleukin-2 (IL-2) or a fragment or a functional fragment or a functional variant thereof, or a combination thereof.

In some embodiments, the at least one cytokine polypeptide comprises interleukin-2 (IL-2) or a fragment thereof. In some embodiments, the at least one cytokine polypeptide is interleukin-2 (IL-2) or a fragment thereof. In some embodiments, the at least one cytokine polypeptide comprises a sequence having at least 75%, 80%, 85%, 90%, 95%, 99%, 99.5%, 99.9%, or 100% sequence identity to the sequence of SEQ ID NO: 2191. In some embodiments, the at least one cytokine polypeptide comprises the sequence of SEQ ID NO: 2191. In some embodiments, the sequence of the at least one cytokine polypeptide is a sequence having at least 75%, 80%, 85%, 90%, 95%, 99%, 99.5%, 99.9%, or 100% sequence identity to the sequence of SEQ ID NO: 2191. In some embodiments, the sequence of the at least one cytokine polypeptide is the sequence of SEQ ID NO: 2191.

In some embodiments, the variant of the at least one cytokine polypeptide comprises an IL-2 variant comprising a mutation. In some embodiments, the mutation comprises an insertion mutation, a deletion mutation, or a substitution mutation. In some embodiments, the mutation comprises the substitution mutation. In some embodiments, the variant comprises an IL-2 variant comprising C125A mutation. In some embodiments, the variant of the at least one cytokine polypeptide is an IL-2 variant comprising a mutation. In some embodiments, the mutation is an insertion mutation, a deletion mutation, or a substitution mutation. In some embodiments, the mutation is the substitution mutation. In some embodiments, the variant is an IL-2 variant comprising C125A mutation. In some embodiments, the variant comprises a sequence having at least 75%, 80%, 85%, 90%, 95%, 99%, 99.5%, 99.9%, or 100% sequence identity to the sequence of SEQ ID NO: 2270. In some embodiments, the variant comprises the sequence of SEQ ID NO: 2270. In some embodiments, the sequence of the variant is a sequence having at least 75%, 80%, 85%, 90%, 95%, 99%, 99.5%, 99.9%, or 100% sequence identity to the sequence of SEQ ID NO: 2270. In some embodiments, the sequence of the variant is the sequence of SEQ ID NO: 2270.

In some embodiments, the first dimerization module comprises a first immunoglobulin constant regions (Fc regions) and the second dimerization module comprises a second Fc region. In some embodiments, the first dimerization module is a first immunoglobulin constant regions (Fc regions) and the second dimerization module is a second Fc region.

In some embodiments, the first Fc region, the second Fc region, or a combination thereof is selected from an IgG1 Fc region or a fragment thereof, an IgG2 Fc region or a fragment thereof, an IgG3 Fc region or a fragment thereof, an IgGA 1 Fc region or a fragment thereof, an IgGA2 Fc region or a fragment thereof, an IgG4 Fc region or a fragment thereof, an IgJ Fc region or a fragment thereof, an IgM Fc region or a fragment thereof, an IgD Fc region or a fragment thereof, and an IgE Fc region or a fragment thereof.

In some embodiments, the first Fc region, the second Fc region, or a combination thereof is selected from a human IgG1 Fc region or a fragment thereof, a human IgG2 Fc region or a fragment thereof, and a human IgG4 Fc region or a fragment thereof.

In some embodiments, the first Fc region, the second Fc region, or a combination thereof comprises an Fc interface with one or more of: a paired cavity-protuberance, an electrostatic interaction, or a strand-exchange, wherein the dimerization of the first Fc region and the second Fc region is enhanced as indicated by a greater ratio of heteromultimer:homomultimer forms relative to a dimerization of Fc regions with a non-engineered interface. In some embodiments, the dimerization of the first Fc region and the second Fc region is enhanced at least by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold. 7 fold, 8 fold, 9 fold, 10 fold, 15 fold, 20 fold, 25 fold, 30 fold, 35 fold, 40 fold, 45 fold, 50 fold, 55 fold, 60 fold, 65 fold. 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface. In some embodiments, the dimerization of the first Fe region and the second Fc region is enhanced at most by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold. 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface. In some embodiments, the dimerization of the first Fc region and the second Fc region is enhanced by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold. 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface.

In some embodiments, the first Fc region, the second Fc region, or a combination thereof comprises an amino acid substitution listed in Table 14.

In some embodiments, the first Fc region, the second Fc region, or a combination thereof comprises an Asn297Ala (N297A) mutation or a Leu234Ala/Leu235Ala (LALA) mutation.

In some embodiments, the first Fc region, the second Fc region, or a combination thereof comprises a sequence having at least 75%, 80%, 85%, 90%, 95%, 99%, 99.5%, 99.9%, or 100% sequence identity to the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649. In some embodiments, the first Fc region, the second Fc region, or a combination thereof comprises the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649.

In some embodiments, the sequence of the first Fc region, the second Fc region, or a combination thereof is a sequence having at least 75%, 80%, 85%, 90%, 95%, 99%, 99.5%, 99.9%, or 100% sequence identity to the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649. In some embodiments, the sequence of the first Fc region, the second Fc region, or a combination thereof is the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649. 1003401In some embodiments, the first TCRβV-binding moiety, the second TCRβV-binding moiety, or a combination thereof binds to one or more of a TCRβV subfamily selected from the group consisting of: (i) the TCRβ V6 subfamily comprising one or more selected from TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01, and TCRβ V6-1*01, and (ii) TCRβ V10 subfamily comprising one or more selected from TCRβ V10-1*01, TCRβ V10-1*02, TCRβ V10-3*01, and TCRβ V10-2*01.

In some embodiments, the first TCRβV-binding moiety and the second TCRβV-binding moiety are same. In some embodiments, the first TCRβV-binding moiety and the second TCRβV-binding moiety are different.

In some embodiments, the first TCRβV-binding moiety and the second TCRβV-binding moiety binds one or more of a TCRβ V6 subfamily member and one or more of a TCRβ V10 subfamily member, respectively.

In some embodiments, the first TCRβV-binding moiety, the second TCRβ 4V-binding moiety, or a combination thereof comprises: (i) a HC CDR1, a HC CDR2 and a HC CDR3 of an amino acid sequence having at least 75%, 80%, 85%, 90%, 95%, 99%, 99.5%, 99.9%, or 100% sequence identity to any one of the CDR1, CDR2, and CDR3 sequences listed in Table L: (ii) a LC CDR1, a LC CDR2, and a LC CDR3 of an amino acid sequence having at least 75%, 80%, 85%, 90%, 95/a, 99%, 99.5%, 99.9%, or 100% sequence identity to any one of the CDR1. CDR2, and CDR3 the sequences listed in Table 1; or (iii) a combination thereof. In some embodiments, the first TCRβV-binding moiety, the second TCRβV-binding moiety, or a combination thereof comprises: (i) a HC CDR 1, a HC CDR2 and a HC CDR3 having any one of the CDR1, CDR2, and CDR3 sequences listed in Table 1; (ii) a LC CDR1, a LC CDR2, and a LC CDR3 having any one of the CDR1, CDR2, and CDR3 the sequences listed in Table 1; or (iii) a combination thereof.

In some embodiments, the first TCRβV-binding moiety, the second TCRβV-binding moiety, or a combination thereof comprises: (i) a HC CDR1, a HC CDR2 and a HC CDR3 of an amino acid sequence having at least 75%, 80%, 85%, 90%, 95%, 99%, 99.5%, 99.9%, or 100% sequence identity to any one of the CDR1, CDR2, and CDR3 sequences listed in Table 1, respectively; (ii) a LC CDR1, a LC CDR2, and a LC CDR3 of an amino acid sequence having at least 75%, 80%, 85%, 90%, 95%, 99%, 99.5%, 99.9%, or 100% sequence identity to any one of the CDR1, CDR2, and CDR3 the sequences listed in Table 1, respectively; or (iii) a combination thereof. In some embodiments, the first TCRβV-binding moiety, the second TCRβV-binding moiety, or a combination thereof comprises: (i) a HC CDR1, a HC CDR2 and a HC CDR3 having any one of the CDR1, CDR2, and CDR3 sequences listed in Table 1, respectively; (ii) a LC CDR1, a LC CDR2, and a LC CDR3 having any one of the CDR1, CDR2, and CDR3 the sequences listed in Table 1, respectively; or (iii) a combination thereof.

In some embodiments, the first TCRβV-binding moiety, the second TCRβV-binding moiety, or a combination thereof comprises: (i) a VH comprising a framework region (FR) comprising a framework 1 (FR1), a framework region 2 (FR2), a framework region 3 (FR3), and a framework region 4 (FR4) that have at least 75/a, 80%, 85%, 90%, 95%, 99%, 99.5%, 99.9%, or 100% sequence identity with a non-murine germline FR1, a non-murine germline FR2, a non-murine germline FR3, and a non-murine germline FR4; (ii) a VL comprising a FR comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4; or (iii) a combination thereof. In some embodiments, the first TCRβV-binding moiety, the second TCRβV-binding moiety, or a combination thereof comprises: (i) a VH comprising a FR comprising a FR1, a FR2, a FR3, and a FR4 having the sequences of a non-murine germline FR 1, a non-murine germline FR2, a non-murine germline FR3, and a non-murine germline FR4; (ii) a VL comprising a FR comprising a FR1, a FR2, a FR3, and a FR4 having the sequences of a non-murine germline FR1, a non-murine germline FR2, a non-murine germline FR3, and a non-murine germline FR4; or (iii) a combination thereof.

In some embodiments, the first TCRβV-binding moiety, the second TCRβV-binding moiety, or a combination thereof comprises: (i) a VH comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4, respectively; (ii) a VL comprising a FR comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4, respectively; or (iii) a combination thereof. In some embodiments, the first TCRβV-binding moiety, the second TCRβV-binding moiety, or a combination thereof comprises: (i) a VH comprising a FR comprising a FR1, a FR2, a FR3, and a FR4 having the sequences of a non-murine germline FR1, a non-murine germline FR2, a non-murine germline FR3, and a non-murine germline FR4, respectively; (ii) a VL comprising a FR comprising a FR1, a FR2, a FR3, and a FR4 having the sequences of a non-murine germline FR1, a non-murine germline FR2, a non-murine germline FR3, and a non-murine germline FR4, respectively: or (iii) a combination thereof.

In some embodiments, the VH comprises the FR3 comprising (i) a Threonine at position 73 according to Kabat numbering: (ii) a Glycine a position 94 according to Kabat numbering; or (iii) a combination thereof. In some embodiments, the VL comprises the FR1 comprising a Phenyalanine at position 10 according to Kabat numbering. In some embodiments, the VL comprises the FR2 comprising (i) a Histidine at position 36 according to Kabat numbering; (ii) an Alanine at position 46 according to Kabat numbering; or (iii) a combination thereof. In some embodiments, the VL comprises the FR3 comprising a Phenyalanine at position 87 according to Kabat numbering.

In some embodiments, the first polypeptide, the second polypeptide, the third polypeptide, the fourth polypeptide, or a combination thereof comprises a heavy chain constant region having a sequence having at least 75%, 80%, 85%, 90%, 95%, 99%, 99.5%, 99.9%, or 100% sequence identity to any one of the sequences listed in Table 3 or a combination thereof. In some embodiments, the first polypeptide, the second polypeptide, the third polypeptide, the fourth polypeptide, or a combination thereof comprises a heavy chain constant region having any one of the sequences listed in Table 3 or a combination thereof. In some embodiments, the first polypeptide, the second polypeptide, the third polypeptide, the fourth polypeptide, or a combination thereof comprises a heavy chain constant region of which sequence is a sequence having at least 75%, 80%, 85%, 90%, 95%, 99%, 99.5%, 99.9%, or 100% sequence identity to any one of the sequences listed in Table 3 or a combination thereof. In some embodiments, the first polypeptide, the second polypeptide, the third polypeptide, the fourth polypeptide, or a combination thereof comprises a heavy chain constant region having any one of the heavy chain constant region sequences listed in Table 3 or a combination thereof. In some embodiments, the first polypeptide, the second polypeptide, the third polypeptide, the fourth polypeptide, or a combination thereof comprises a heavy chain constant region of an IgM or a fragment thereof. In some embodiments, the heavy chain constant region of the IgM comprises a sequence having at least 75%, 80%, 85%, 90%, 95%, 99%, 99.5%, 99.9%, or 100% sequence identity to the sequence of SEQ ID NO: 73. In some embodiments, the heavy chain constant region of the IgM comprises the sequence of SEQ ID NO: 73. In some embodiments, the sequence of the heavy chain constant region of the IgM is the sequence of SEQ ID NO: 73.

In some embodiments, the first polypeptide, the second polypeptide, the third polypeptide, the fourth polypeptide, or a combination thereof comprises a heavy chain constant region of an IgJ or a fragment thereof. In some embodiments, the heavy chain constant region of the IgJ comprises a sequence having at least 75%, 80%, 85%, 90%, 95%, 99%, 99.5%, 99.9%, or 100% sequence identity to the sequence of SEQ ID NO: 76. In some embodiments, the heavy chain constant region of the IgJ comprises the sequence of SEQ ID NO: 76. In some embodiments, the sequence of the heavy chain constant region of the IgJ is the sequence of SEQ ID NO: 76.

In some embodiments, the first polypeptide, the second polypeptide, the third polypeptide, the fourth polypeptide, or a combination thereof comprises a heavy chain constant region of an IgGA 1 or a fragment thereof. In some embodiments, the heavy chain constant region of the IgGA1 comprises a sequence having at least 75%, 80%, 85%, 90%, 95%, 99%, 99.5%, 99.9%, or 100% sequence identity to the sequence of SEQ ID NO: 74. In some embodiments, the heavy chain constant region of the IgGA1 comprises the sequence of SEQ ID NO: 74. In some embodiments, the sequence of the heavy chain constant region of the IgGA 1 is the sequence of SEQ ID NO: 74.

In some embodiments, the first polypeptide, the second polypeptide, the third polypeptide, the fourth polypeptide, or a combination thereof comprises a heavy chain constant region of an IgGA2 or a fragment thereof. In some embodiments, the heavy chain constant region of the IgGA2 comprises a sequence having at least 75%, 80%, 85%, 90%, 95%, 99%, 99.5%, 99.9%, or 100% sequence identity to the sequence of SEQ ID NO: 75. In some embodiments, the heavy chain constant region of the IgGA2 comprises the sequence of SEQ ID NO: 75. In some embodiments, the sequence of the heavy chain constant region of the IgGA2 is the sequence of SEQ ID NO: 75.

In some embodiments, the first polypeptide, the second polypeptide, the third polypeptide, the fourth polypeptide, or a combination thereof comprises a heavy chain constant region of an IgG1 or a fragment thereof. In some embodiments, the heavy chain constant region of the IgG1 comprises a sequence having at least 75%, 80%, 85%, 90%, 95%, 99%, 99.5%, 99.9%, or 100% sequence identity to the sequence of SEQ ID NO: 41. In some embodiments, the heavy chain constant region of the IgG1 comprises the sequence of SEQ ID NO: 41. In some embodiments, the sequence of the heavy chain constant region of the IgG1 is the sequence of SEQ ID NO: 41. In some embodiments, the heavy chain constant region of the IgG1 comprises a sequence having at least 75%, 80%, 85%, 90%, 95%, 99%, 99.5%, 99.9%, or 100% sequence identity to the sequence of SEQ ID NO: 3645. In some embodiments, the heavy chain constant region of the IgG1 comprises the sequence of SEQ ID NO: 3645. In some embodiments, the sequence of the heavy chain constant region of the IgG1 is the sequence of SEQ ID NO: 3645.

In some embodiments, the first polypeptide, the second polypeptide, the third polypeptide, the fourth polypeptide, or a combination thereof comprises a light chain constant region having a sequence having at least 75%, 80%, 85%, 90%, 95%, 99%, 99.5%, 99.9%, or 100% sequence identity to any one of the sequences listed in Table 3 or a combination thereof. In some embodiments, the first polypeptide, the second polypeptide, the third polypeptide, the fourth polypeptide, or a combination thereof comprises a light chain constant region having any one of the sequences listed in Table 3 or a combination thereof. In some embodiments, the first polypeptide, the second polypeptide, the third polypeptide, the fourth polypeptide, or a combination thereof comprises a light chain constant region having any one of the light chain constant region sequences listed in Table 3 or a combination thereof.

In some embodiments, the first polypeptide, the second polypeptide, the third polypeptide, the fourth polypeptide, or a combination thereof comprises a light chain constant region of a kappa chain or a fragment thereof. In some embodiments, the light chain constant region of a kappa chain comprises a light chain constant region sequence listed in Table 3.

In some embodiments, the light chain constant region of a kappa chain comprises a sequence having at least 75%, 80%, 85%, 90%, 95%, 99%, 99.5%, 99.9%, or 100% sequence identity to the sequence of SEQ ID NO: 39 or SEQ ID NO: 3644. In some embodiments, the light chain constant region of a kappa chain comprises the sequence of SEQ ID NO: 39 or SEQ ID NO: 3644. In some embodiments, the sequence of the light chain constant region of a kappa chain is a sequence having at least 75%, 80%, 85%, 90%, 95%, 99%, 99.5/a, 99.9%, or 100% sequence identity to the sequence of SEQ ID NO: 39 or SEQ ID NO: 3644. In some embodiments, the sequence of the light chain constant region of a kappa chain is the sequence of SEQ ID NO: 39 or SEQ ID NO: 3644.

In some embodiments, the first TCRβV-binding moiety, the second TCRβV-binding moiety, or a combination thereof comprises a light chain comprising a FR1 comprising: (i) an Aspartic Acid at position 1 according to Kabat numbering; (ii) an Asparagine at position 2 according to Kabat numbering; (iii) a Leucine at position 4 according to Kabat numbering; or (iv) a combination thereof.

In some embodiments, the first TCRβV-binding moiety, the second TCRβV-binding moiety, or a combination thereof comprises a light chain comprising a FR3 comprising: (i) a Glycine at position 66 according to Kabat numbering; (ii) an Asparagine at position 69 according to Kabat numbering; (iii) a Tyrosine at position 71 according to Kabat numbering; or (iv) a combination thereof

In some embodiments, the first TCRβV-binding moiety, the second TCRβV-binding moiety, or a combination thereof binds to an outward facing region on a TCRβV protein. In some embodiments, the outward facing region on the TCRβV protein comprises a structurally conserved region of TCRβV having a similar structure across one or more TCRβV subfamilies.

### Cytokine Molecules

In some embodiments, the multifunctional molecule includes a cytokine molecule. As used herein, a “cytokine molecule” or a “cytokine polypeptide” as interchangeably used herein, refers to full length, a fragment or a variant of a cytokine; a cytokine further comprising a receptor domain, e.g., a cytokine receptor dimerizing domain; or an agonist of a cytokine receptor, e.g., an antibody molecule (e.g., an agonistic antibody) to a cytokine receptor, that elicits at least one activity of a naturally-occurring cytokine. In some embodiments the cytokine molecule is interleukin-2 (IL-2), or a fragment or variant thereof, or a combination. The cytokine molecule can be a monomer or a dimer. In embodiments, the cytokine molecule can further include a cytokine receptor dimerizing domain. In other embodiments, the cytokine molecule is an agonist of a cytokine receptor.

Cytokines are generally polypeptides that influence cellular activity, for example, through signal transduction pathways. Accordingly, a cytokine of the multispecific or multifunctional polypeptide is useful and can be associated with receptor-mediated signaling that transmits a signal from outside the cell membrane to modulate a response within the cell. Cytokines are proteinaceous signaling compounds that are mediators of the immune response. They control many different cellular functions including proliferation, differentiation and cell survival/apoptosis: cytokines are also involved in several pathophysiological processes including viral infections and autoimmune diseases. Cytokines are synthesized under various stimuli by a variety of cells of both the innate (monocytes, macrophages, dendritic cells) and adaptive (T- and B-cells) immune systems. Cytokines can be classified into two groups: pro- and anti-inflammatory. Pro-inflammatory cytokines, including IFNγ, IL-1, IL-6 and TNF-alpha, are predominantly derived from the innate immune cells and Th1 cells. Anti-inflammatory cytokines, including IL-10, IL-4, IL-13 and IL-5, are synthesized from Th2 immune cells.

Provided herein are, inter alia, multispecific (e.g., bi-, tri-, quad-specific) or multifunctional molecules, that include, e.g., arc engineered to contain, one or more cytokine molecules, e.g., immunomodulatory (e.g., proinflammatory) cytokines and variants, e.g., functional variants, thereof. Accordingly, in some embodiments, the cytokine molecule is an interleukin or a variant, e.g., a functional variant thereof. In some embodiments the interleukin is a proinflammatory interleukin. In some embodiments the interleukin is interleukin-2 (IL-2). In some embodiments, the cytokine molecule is a proinflammatory cytokine.

In certain embodiments, the cytokine is a single chain cytokine. In certain embodiments, the cytokine is a multichain cytokine (e.g., the cytokine comprises 2 or more (e.g., 2) polypeptide chains.

Examples of useful cytokines include, but are not limited to, IL-2. In some embodiments the cytokine of the multispecific or multifunctional polypeptide is IL-2.In certain embodiments the cytokine is mutated to remove N- and/or O-glycosylation sites. Elimination of glycosylation increases homogeneity of the product obtainable in recombinant production.

In some embodiments, the cytokine of the multispecific or multifunctional polypeptide is IL-2. In a specific embodiment, the IL-2 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity. In another particular embodiment the IL-2 cytokine is a mutant IL-2 cytokine having reduced binding affinity to the .alpha.-subunit of the IL-2 receptor. Together with the .beta.- and .gamma.-subunits (also known as CD122 and CD132, respectively), the .alpha.-subunit (also known as CD25) forms the heterotrimeric high-affinity IL-2 receptor, while the dimeric receptor consisting only of the β- and γ-subunits is termed the intermediate-affinity IL-2 receptor. As described in PCT patent application number PCT/EP2012/051991, which is incorporated herein by reference in its entirety, a mutant IL-2 polypeptide with reduced binding to the .alpha.-subunit of the IL-2 receptor has a reduced ability to induce IL-2 signaling in regulatory T cells, induces less activation-induced cell death (AICD) in T cells, and has a reduced toxicity profile in vivo, compared to a wild-type IL-2 polypeptide. The use of such an cytokine with reduced toxicity is particularly advantageous in a multispecific or multifunctional polypeptide according to the invention, having a long serum half-life due to the presence of an Fc domain. In some embodiments, the mutant IL-2 cytokine of the multispecific or multifunctional polypeptide according to the invention comprises at least one amino acid mutation that reduces or abolishes the affinity of the mutant IL-2 cytokine to the .alpha.-subunit of the IL-2 receptor (CD25) but preserves the affinity of the mutant IL-2 cytokine to the intermediate-affinity IL-2 receptor (consisting of the β and γ subunits of the IL-2 receptor), compared to the non-mutated IL-2 cytokine. In some embodiments the one or more amino acid mutations are amino acid substitutions. In a specific embodiment, the mutant IL-2 cytokine comprises one, two or three amino acid substitutions at one, two or three position(s) selected from the positions corresponding to residue 42, 45, and 72 of human IL-2. In a more specific embodiment, the mutant IL-2 cytokine comprises three amino acid substitutions at the positions corresponding to residue 42, 45 and 72 of human IL-2. In an even more specific embodiment, the mutant IL-2 cytokine is human IL-2 comprising the amino acid substitutions F42A, Y45A and L72G. In some embodiments the mutant IL-2 cytokine additionally comprises an amino acid mutation at a position corresponding to position 3 of human IL-2, which eliminates the O-glycosylation site of IL-2. Particularly, said additional amino acid mutation is an amino acid substitution replacing a threonine residue by an alanine residue. A particular mutant IL-2 cytokine useful in the invention comprises four amino acid substitutions at positions corresponding to residues 3, 42, 45 and 72 of human IL-2. Specific amino acid substitutions are T3A, F42A, Y45A and L72G. As demonstrated in PCT patent application number PCT/EP2012/051991 and in the appended Examples, said quadruple mutant IL-2 polypeptide (IL-2 qm) exhibits no detectable binding to CD25, reduced ability to induce apoptosis in T cells, reduced ability to induce IL-2 signaling in T.sub.reg cells, and a reduced toxicity profile in vivo. However, it retains ability to activate IL-2 signaling in effector cells, to induce proliferation of effector cells, and to generate IFN-γ as a secondary cytokine by NK cells.

The IL-2 or mutant IL-2 cytokine according to any of the above embodiments may comprise additional mutations that provide further advantages such as increased expression or stability. For example, the cysteine at position 125 may be replaced with a neutral amino acid such as alanine, to avoid the formation of disulfide-bridged IL-2 dimers. Thus, in certain embodiments the IL-2 or mutant IL-2 cytokine of the multispecific or multifunctional polypeptide according to the invention comprises an additional amino acid mutation at a position corresponding to residue 125 of human IL-2. In some embodiments said additional amino acid mutation is the amino acid substitution C125A. 1003661In a specific embodiment the IL-2 cytokine of the multispecific or multifunctional polypeptide comprises the polypeptide sequence of SEQ ID NO: 2270 [APTSSSTKKTQLQLEHLLLDLQMILNGINNYKNPKLTRMLTFKFYMPKKATELKHLQCLEEELK PLEEVLNLAQSKNFHLRPRDLISNINVIVLELKGSETTFMCEYADETATIVEFLNRWITFAQSIISTL T].

In another specific embodiment the IL-2 cytokine of the multispecific or multifunctional polypeptide comprises the polypeptide sequence of SEQ ID NO: 2280 [A PASSSTKKTQLQLEHLLLDLQMILNGINNYKNPKLTRMLTAKFAMPKKATELKHLQCLEEELK PLEEVLNGAQSKNFHLRPRDLISNINVIVLELKGSETTFMCEYADETATIVEFLNRWITFAQSIISTL T].

Mutant cytokine molecules useful as effector moieties in the multispecific or multifunctional polypeptide can be prepared by deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing. Substitution or insertion may involve natural as well as non-natural amino acid residues. Amino acid modification includes well known methods of chemical modification such as the addition or removal of glycosylation sites or carbohydrate attachments, and the like.

In some embodiments, the multispecific or multifunctional polypeptide of the invention binds to an cytokine receptor with a dissociation constant (K_{D}) that is at least about 1, 1.5, 2, 2.5, 3, 3.5, 4, 4.5, 5, 5.5, 6, 6.5, 7, 7.5, 8, 8.5, 9, 9.5 or 10 times greater than that for a control cytokine. In another embodiment, the multispecific or multifunctional polypeptide binds to an cytokine receptor with a K, that is at least 2, 3, 4, 5, 6, 7, 8, 9, or 10 times greater than that for a corresponding multispecific or multifunctional polypeptide comprising two or more effector moieties. In another embodiment, the multispecific or multifunctional polypeptide binds to an cytokine receptor with a dissociation constant K_{D}that is about 10 times greater than that for a corresponding the multispecific or multifunctional polypeptide comprising two or more cytokines.

In some embodiments, the multispecific molecules as described herein include a cytokine molecule. In embodiments, the cytokine molecule includes a full length, a fragment or a variant of a cytokine; a cytokine receptor domain, e.g., a cytokine receptor dimerizing domain: or an agonist of a cytokine receptor, e.g., an antibody molecule (e.g., an agonistic antibody) to a cytokine receptor.

In other embodiments, the cytokine molecule is IL-2, e.g., human IL-2 (e.g., comprising the amino acid sequence: APTSSSTKKTQLQLEHLLLDLQMILNGINNYKNPKLTRMLTFKFYMPKKATELKHLQCLEEELKP LEEVLNLAQSKNFHLRPRDLISNINVIVLELKGSETIFMCEYADETATIVEFLNRWITFCQSIISTLT (SEQ ID NO: 2191), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO:2191).

### Immune Cell Engagers

In some embodiments, the multifunctional molecule further includes an immune cell engager. “An immune cell engager” refers to one or more binding specificities that bind and/or activate an immune cell, e.g., a cell involved in an immune response. In embodiments, the immune cell is chosen from a T cell, an NK cell, a B cell, a dendritic cell, and/or the macrophage cell. The immune cell engager can be an antibody molecule, a receptor molecule (e.g., a full length receptor, receptor fragment, or fusion thereof (e.g., a receptor-Fc fusion)), or a ligand molecule (e.g., a full length ligand, ligand fragment, or fusion thereof (e.g., a ligand-Fc fusion)) that binds to the immune cell antigen (e.g., the T cell, the NK cell antigen, the B cell antigen, the dendritic cell antigen, and/or the macrophage cell antigen). In embodiments, the immune cell engager specifically binds to the target immune cell, e.g., binds preferentially to the target immune cell. For example, when the immune cell engager is an antibody molecule, it binds to an immune cell antigen (e.g., a T cell antigen, an NK cell antigen, a B cell antigen, a dendritic cell antigen, and/or a macrophage cell antigen) with a dissociation constant of less than about 10 nM.

The immune cell engagers, e.g., first and/or second immune cell engager, of the multispecific or multifunctional molecules as described herein can mediate binding to, and/or activation of, an immune cell, e.g., an immune effector cell. In some embodiments, the immune cell is chosen from a T cell, an NK cell, a B cell, a dendritic cell, or a macrophage cell engager, or a combination thereof. In some embodiments, the immune cell engager is chosen from one, two, three, or all of a T cell engager, NK cell engager, a B cell engager, a dendritic cell engager, or a macrophage cell engager, or a combination thereof. The immune cell engager can be an agonist of the immune system. In some embodiments, the immune cell engager can be an antibody molecule, a ligand molecule (e.g., a ligand that further comprises an immunoglobulin constant region, e.g., an Fc region), a small molecule, a nucleotide molecule.

### Antibody Molecules

In some embodiments, the antibody molecule binds to a cancer antigen, e.g., a tumor antigen or a stromal antigen. In some embodiments, the cancer antigen is, e.g., a mammalian, e.g., a human, cancer antigen. In other embodiments, the antibody molecule binds to an immune cell antigen, e.g., a mammalian, e.g., a human, immune cell antigen. For example, the antibody molecule binds specifically to an epitope, e.g., linear or conformational epitope, on the cancer antigen or the immune cell antigen.

In some embodiments, an antibody molecule is a monospecific antibody molecule and binds a single epitope. E.g., a monospecific antibody molecule having a plurality of immunoglobulin variable domain sequences, each of which binds the same epitope.

In some embodiments, an antibody molecule is a multispecific or multifunctional antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a multifunctional antibody molecule comprises a third, fourth or fifth immunoglobulin variable domain. In some embodiments, a multifunctional antibody molecule is a bispecific antibody molecule, a trispecific antibody molecule, or a tetraspecific antibody molecule.

In some embodiments, a multifunctional antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In some embodiments, a bispecific antibody molecule comprises a half antibody having binding specificity for a first epitope and a half antibody having binding specificity for a second epitope. In some embodiments, a bispecific antibody molecule comprises a half antibody, or fragment thereof, having binding specificity for a first epitope and a half antibody, or fragment thereof, having binding specificity for a second epitope. In some embodiments, a bispecific antibody molecule comprises a scFv or a Fab, or fragment thereof, have binding specificity for a first epitope and a scFv or a Fab, or fragment thereof, have binding specificity for a second epitope.

In some embodiments, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable domain sequence (abbreviated herein as VH), and a light (L) chain variable domain sequence (abbreviated herein as VL). In some embodiments, an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may, be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains: (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain: (vi) a camelid or camelized variable domain: (vii) a single chain Fv (scFv), see e.g., Bird et al. (1988) Science 242:423426; and Huston et al. (1988) Proc. Natl. Acad Sci. USA 85:5879-5883), (viii) a single domain antibody. These antibody fragments are obtained using conventional techniques known to those with skill in the art, and the fragments are screened for utility in the same manner as are intact antibodies.

Antibody molecules include intact molecules as well as functional fragments thereof. Constant regions of the antibody molecules can be altered. e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242: Chothia. C. er al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S, and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani er al., (1997) JMB 273, 927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3).

Each VH and VL typically includes three CDRs and four FRs, arranged from amino-terminus to carboxy-terminus in the following order: FR1, CDR1, FR2, CDR2, FR3, CDR3, FR4.

The antibody molecule can be a polyclonal or a monoclonal antibody.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

The antibody can be recombinantly produced, e.g., produced by phage display or by combinatorial methods, or by yeast display.

Phage display and combinatorial methods for generating antibodies are known in the art (as described in, e.g., Ladner eat. U.S. Pat. No. 5,223,409; Kang et at. International Publication No. WO 92/18619; Dower et at. International Publication No. WO 91/17271; Winter et al International Publication WO 92/20791; Markland et at. International Publication No. WO 92/15679; Breitling et al. International Publication WO 93/01288; McCafferty et at. International Publication No. WO 92/01047; Garrard et at. International Publication No. WO 92/09690; Ladner et al. International Publication No. WO 90/02809; Fuchs et at. (1991) Bio/Technology 9:1370-1372: Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

The yeast display method for generating or identifying antibodies is known in the art, e.g., as described in Chao et al. (2006) Nature Protocols 1(2):755-68, the entire contents of which is incorporated by reference herein.

In some embodiments, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741: Lonberg et al. International Application WO 92/03918; Kay et al. International Application 92/03917: Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody molecule can be one in which the variable region, or a portion thereof, e.g., the CDRs, are generated in a non-human organism, e.g., a rat or mouse. Chimeric, CDR-grafted, and humanized antibodies are within the invention. Antibody molecules generated in a non-human organism, e.g., a rat or mouse, and then modified, e.g., in the variable framework or constant region, to decrease antigenicity in a human are within the invention.

An “effectively human” protein is a protein that does substantially not evoke a neutralizing antibody response. e.g., the human anti-murine antibody (HAMA) response. HAMA can be problematic in a number of circumstances. e.g., if the antibody molecule is administered repeatedly, e.g., in treatment of a chronic or recurrent disease condition. A HAMA response can make repeated antibody administration potentially ineffective because of an increased antibody clearance from the serum (see. e.g., Saleh et al., Cancer Immunol. Immunother., 32:180-190 (1990)) and also because of potential allergic reactions (see. e.g., LoBuglio et al., Hybridoma, 5:5117-5123 (1986)).

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043): Liu et al. (1987) PNAS 84:3439-3443; Liu et al., 1987, J. Immunol. 139:3521-3526: Sun et al. (1987) PNAS 84:214-218; Nishimura et al., 1987, Canc. Res. 47:999-1005: Wood et al. (1985) Nature 314:446-449; and Shaw er al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding to the antigen. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In some embodiments, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody molecule can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which arc hereby incorporated by reference).

Humanized or CDR-grafted antibody molecules can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525: Verhoeyan et al. 1988 Science 239:1534: Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibody molecules in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In some embodiments the antibody has: effector function; and can fix complement. In other embodiments the antibody does not: recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

### CDR-Grafted Scaffolds

In some embodiments, the antibody molecule is a CDR-grafted scaffold domain. In some embodiments, the scaffold domain is based on a fibronectin domain, e.g., fibronectin type III domain. The overall fold of the fibronectin type III (Fn3) domain is closely related to that of the smallest functional antibody fragment, the variable domain of the antibody heavy chain. There are three loops at the end of Fn3; the positions of BC, DE and FG loops approximately correspond to those of CDR1, 2 and 3 of the VH domain of an antibody. Fn3 does not have disulfide bonds; and therefore Fn3 is stable under reducing conditions, unlike antibodies and their fragments (see, e.g., WO 98/56915; WO 01/64942: WO 00/34784). An Fn3 domain can be modified (e.g., using CDRs or hypervariable loops described herein) or varied, e.g., to select domains that bind to an antigen/marker/cell described herein.

In some embodiments, a scaffold domain, e.g., a folded domain, is based on an antibody, e.g., a “minibody” scaffold created by deleting three beta strands from a heavy chain variable domain of a monoclonal antibody (see, e.g., Tramontano et al., 1994, J Mol. Recognit. 7:9; and Martin et al., 1994, EMBO J. 13:5303-5309). The “minibody” can be used to present two hypervariable loops. In some embodiments, the scaffold domain is a V-like domain (see, e.g., Coia et al. WO 99/45110) or a domain derived from tendamistatin, which is a 74 residue, six-strand beta sheet sandwich held together by two disulfide bonds (see, e.g., McConnell and Hoess, 1995, J Mol. Biol. 250:460). For example, the loops of tendamistatin can be modified (e.g., using CDRs or hypervariable loops) or varied, e.g., to select domains that bind to a marker/antigen/cell described herein. Another exemplary scaffold domain is a beta-sandwich structure derived from the extracellular domain of CTLA-4 (see, e.g., WO 00/60070).

Other exemplary scaffold domains include but are not limited to T-cell receptors: MHC proteins: extracellular domains (e.g., fibronectin Type III repeats, EGF repeats): protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains). See, e.g., US 20040009530 and U.S. Pat. No. 7,501,121, incorporated herein by reference.

In some embodiments, a scaffold domain is evaluated and chosen, e.g., by one or more of the following criteria: (1) amino acid sequence, (2) sequences of several homologous domains, (3) 3-dimensional structure, and/or (4) stability data over a range of pH, temperature, salinity, organic solvent, oxidant concentration. In some embodiments, the scaffold domain is a small, stable protein domain, e.g., a protein of less than 100, 70, 50, 40 or 30 amino acids. The domain may include one or more disulfide bonds or may chelate a metal, e.g., zinc.

### Antibody-Based Fusions

A variety of formats can be generated which contain additional binding entities attached to the N or C terminus of antibodies. These fusions with single chain or disulfide stabilized Fvs or Fabs result in the generation of tetravalent molecules with bivalent binding specificity for each antigen. Combinations of scFvs and scFabs with IgGs enable the production of molecules which can recognize three or more different antigens.

### Antibody-Fab Fusion

Antibody-Fab fusions are bispecific antibodies comprising a traditional antibody to a first target and a Fab to a second target fused to the C terminus of the antibody heavy chain. Commonly the antibody and the Fab will have a common light chain. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Antibody-scFv Fusion

Antibody-scFv Fusions are bispecific antibodies comprising a traditional antibody and a scFv of unique specificity fused to the C terminus of the antibody heavy chain. The scFv can be fused to the C terminus through the Heavy Chain of the scFv either directly or through a linker peptide. Antibody fusions can be produced by (I) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Variable Domain Immunoglobulin DVD

A related format is the dual variable domain immunoglobulin (DVD), which are composed of VH and VL domains of a second specificity place upon the N termini of the V domains by shorter linker sequences.

Other exemplary multifunctional antibody formats include, e.g., those described in the following US20160114057A1, US20130243775A1, US20140051833, US20130022601, US20150017187A1, US20120201746A1, US20150133638A1, US20130266568A1, US20160145340A1, WO2015127158A1, US20150203591A1. US20140322221A 1, US20130303396A1, US20110293613, US20130017200A1, US20160102135A1, WO2015197598A2, WO2015197582A1, U.S. Pat. No. 9,359,437, US20150018529, WO2016115274A1, WO2016087416A1, US20080069820A1, U.S. Pat. Nos. 9,145,588B, 7,919,257, and US20150232560A 1. Exemplary multifunctional molecules utilizing a full antibody-Fab/scFab format include those described in the following, U.S. Pat. No. 9,382,323B2, US20140072581A 1, US20140308285A 1, US20130165638A1, US20130267686A1, US20140377269A1, U.S. Pat. No. 7,741,446B2, and WO1995009917A1. Exemplary multifunctional molecules utilizing a domain exchange format include those described in the following, US20150315296A1, WO2016087650A1, US20160075785A1, WO2016016299A1, US20160130347A 1, US20150166670, U.S. Pat. No. 8,703,132B2, US20100316645, U.S. Pat. No. 8,227,577B2, US20130078249.

### Fc-Containing Multifunctional Molecules

In some embodiments, the multifunctional molecules as described herein includes an immunoglobulin constant region (e.g., an Fc region). Exemplary Fc regions can be chosen from the heavy chain constant regions of IgG1, IgG2, IgG3 or IgG4: more particularly, the heavy chain constant region of human IgG1, IgG2, IgG3, or IgG4.

In some embodiments, the immunoglobulin chain constant region (e.g., the Fc region) is altered, e.g., mutated, to increase or decrease one or more of: Fc receptor binding, antibody glycosylation, the number of cysteine residues, effector cell function, or complement function.

In other embodiments, an interface of a first and second immunoglobulin chain constant regions (e.g., a first and a second Fc region) is altered, e.g., mutated, to increase or decrease dimerization, e.g., relative to a non-engineered interface, e.g., a naturally-occurring interface. For example, dimerization of the immunoglobulin chain constant region (e.g., the Fc region) can be enhanced by providing an Fc interface of a first and a second Fc region with one or more of: a paired protuberance-cavity (“knob-in-a hole”), an electrostatic interaction, or a strand-exchange, such that a greater ratio of heteromultimer to homomultimer forms, e.g., relative to a non-engineered interface.

In some embodiments, the multifunctional molecules include a paired amino acid substitution at a position chosen from one or more of 347, 349, 350, 351, 366, 368, 370, 392, 394, 395, 397, 398, 399, 405, 407, or 409, e.g., of the Fc region of human IgG1 For example, the immunoglobulin chain constant region (e.g., Fc region) can include a paired an amino acid substitution chosen from: T366S, L368A, or Y407V (e.g., corresponding to a cavity or hole), and T366W (e.g., corresponding to a protuberance or knob).

In other embodiments, the multifunctional molecule includes a half-life extender, e.g., a human serum albumin or an antibody molecule to human serum albumin.

In some embodiments, Fc contains exemplary Fc modifications listed in Table 14.

### Heterodimerized Antibody Molecules & Methods of Making

Various methods of producing multifunctional antibodies have been disclosed to address the problem of incorrect heavy chain pairing. Exemplary methods are described below. Exemplary multifunctional antibody formats and methods of making said multifunctional antibodies are also disclosed in e.g., Speiss et al. Molecular Immunology 67 (2015) 95-106; and Klein et al mAbs 4:6, 653-663; November/December 2012: the entire contents of each of which are incorporated by reference herein.

Heterodimerized bispecific antibodies are based on the natural IgG structure, wherein the two binding arms recognize different antigens. IgG derived formats that enable defined monovalent (and simultaneous) antigen binding are generated by forced heavy chain heterodimerization, combined with technologies that minimize light chain mispairing (e.g., common light chain). Forced heavy chain heterodimerization can be obtained using, e.g., knob-in-hole OR strand exchange engineered domains (SEED).

### Knob-In-Hole

Knob-in-Hole as described in U.S. Pat. No. 5,731,116. U.S. Pat. No. 7,476,724 and Ridgway, J. et al. (1996) Prot. Engineering 9(7): 617-621, broadly involves: (1) mutating the CH3 domain of one or both antibodies to promote heterodimerization; and (2) combining the mutated antibodies under conditions that promote heterodimerization. “Knobs” or “protuberances” are typically created by replacing a small amino acid in a parental antibody with a larger amino acid (e.g., T366Y or T366W): “Holes” or “cavities” are created by replacing a larger residue in a parental antibody with a smaller amino acid (e.g., Y407T, T366S, L368A and/or Y407V).

For bispecific antibodies including an Fc domain, introduction of specific mutations into the constant region of the heavy chains to promote the correct heterodimerization of the Fc portion can be utilized. Several such techniques are reviewed in Klein et al. (mAbs (2012) 4:6, 1-11), the contents of which are incorporated herein by reference in their entirety. These techniques include the “knobs-into-holes” (KiH) approach which involves the introduction of a bulky residue into one of the CH3 domains of one of the antibody heavy chains. This bulky residue fits into a complementary “hole” in the other CH3 domain of the paired heavy chain so as to promote correct pairing of heavy chains (see e.g., U.S. Pat. No. 7,642,228).

Exemplary KiH mutations include S354C, T366W in the “knob” heavy chain and Y349C. T366S, L368A, Y407V in the “hole” heavy chain. Other exemplary KiH mutations are provided in Table 4, with additional optional stabilizing Fc cysteine mutations.

Other Fc mutations are provided by Igawa and Tsunoda who identified 3 negatively charged residues in the CH3 domain of one chain that pair with three positively charged residues in the CH3 domain of the other chain. These specific charged residue pairs are: E356-K439, E357-K370, D399-K409 and vice versa. By introducing at least two of the following three mutations in chain A: E356K, E357K and D399K, as well as K370E, K409D, K439E in chain B, alone or in combination with newly identified disulfide bridges, they were able to favor very efficient heterodimerization while suppressing homodimerization at the same time (Martens T et al. A novel one-armed antic-Met antibody inhibits glioblastoma growth in vivo. Clin Cancer Res 2006; 12:6144-52; PMID:17062691). Xencor defined 41 variant pairs based on combining structural calculations and sequence information that were subsequently screened for maximal heterodimerization, defining the combination of S364H, F405A (HA) on chain A and Y349T, T394F on chain B (TF) (Moore G L et al. A novel bispecific antibody format enables simultaneous bivalent and monovalent co-engagement of distinct target antigens. MAbs 2011; 3:546-57; PMID: 22123055).

Other exemplary Fc mutations to promote heterodimerization of multifunctional antibodies include those described in the following references, the contents of each of which is incorporated by reference herein, WO2016071377A1, US20140079689A 1, US20160194389A 1, US20160257763, WO2016071376A2, WO2015107026A1, WO2015107025A1, WO2015107015A1, US20150353636A1, US20140199294A1, U.S. Pat. No. 7,750,128B2, US20160229915A1, US20150344570A1, U.S. Pat. No. 8,003,774A1, US20150337049A1, US20150175707A1, US20140242075A1, US20130195849A1, US20120149876A1, US20140200331A 1, US9309311B2, U.S. Pat. No. 8,586,713, US20140037621A1, US20130178605A1, US20140363426A1, US20140051835A1 and US20110054151A1.

Stabilizing cysteine mutations have also been used in combination with KiH and other Fc heterodimerization promoting variants, see e.g., U.S. Pat. No. 7,183,076. Other exemplary cysteine modifications include, e.g., those disclosed in US20140348839A1. U.S. Pat. No. 7,855,275B2, and U.S. Pat. No. 9,000,130B2.

### Strand Exchange Engineered Domains (SEED)

Heterodimeric Fc platform that support the design of bispecific and asymmetric fusion proteins by devising strand-exchange engineered domain (SEED) C(H)3 heterodimers are known. These derivatives of human IgG and IgA C(H)3 domains create complementary human SEED C(H)3 heterodimers that are composed of alternating segments of human IgA and IgG C(H)3 sequences. The resulting pair of SEED C(H)3 domains preferentially associates to form heterodimers when expressed in mammalian cells. SEEDbody (Sb) fusion proteins consist of [IgG1 hinge]-C(H)2-[SEED C(H)3], that may be genetically linked to one or more fusion partners (see e.g., Davis J H et al. SEEDbodies: fusion proteins based on strand exchange engineered domain (SEED) CH3 heterodimers in an Fc analogue platform for asymmetric binders or immunofusions and bispecific antibodies. Protein Eng Des Sel 2010; 23:195-202; PMID:20299542 and U.S. Pat. No. 8,871,912. The contents of each of which are incorporated by reference herein).

### Fc-Containing Entities (Mini-Antibodies)

Fc-containing entities, also known as mini-antibodies, can be generated by fusing scFv to the C-termini of constant heavy region domain 3 (CH3-scFv) and/or to the hinge region (scFv-hinge-Fc) of an antibody with a different specificity. Trivalent entities can also be made which have disulfide stabilized variable domains (without peptide linker) fused to the C-terminus of CH3 domains of IgGs.

### Duobody

“Duobody” technology to produce bispecific antibodies with correct heavy chain pairing are known. The DuoBody technology involves three basic steps to generate stable bispecific human IgG1antibodies in a post-production exchange reaction. In a first step, two IgG1s, each containing single matched mutations in the third constant (CH3) domain, are produced separately using standard mammalian recombinant cell lines. Subsequently, these IgG1 antibodies are purified according to standard processes for recovery and purification. After production and purification (post-production), the two antibodies are recombined under tailored laboratory conditions resulting in a bispecific antibody product with a very high yield (typically >95%) (see e.g., Labrijn et al. PNAS 2013:110(13):5145-5150 and Labrijn et al. Nature Protocols 2014; 9(10):2450-63, the contents of each of which are incorporated by reference herein).

### Electrostatic Interactions

Methods of making multifunctional antibodies using CH3 amino acid changes with charged amino acids such that homodimer formation is electrostatically unfavorable are disclosed. EP1870459 and WO 2009089004 describe other strategies for favoring heterodimer formation upon co-expression of different antibody domains in a host cell. In these methods, one or more residues that make up the heavy chain constant domain 3 (CH3), CH3-CH3 interfaces in both CH3 domains are replaced with a charged amino acid such that homodimer formation is electrostatically unfavorable and heterodimerization is electrostatically favorable. Additional methods of making multifunctional molecules using electrostatic interactions are described in the following references, the contents of each of which is incorporated by reference herein, include US20100015133, U.S. Pat. No. 8,592,562B2, U.S. Pat. No. 9,200,060B2, US20140154254A1, and U.S. Pat. No. 9,358,286A1.

### Common Light Chain

Light chain mispairing needs to be avoided to generate homogenous preparations of bispecific IgGs. One way to achieve this is through the use of the common light chain principle, i.e. combining two binders that share one light chain but still have separate specificities. An exemplary method of enhancing the formation of a desired bispecific antibody from a mixture of monomers is by providing a common variable light chain to interact with each of the heteromeric variable heavy chain regions of the bispecific antibody. Compositions and methods of producing bispecific antibodies with a common light chain as disclosed in, e.g., U.S. Pat. No. 7,183,076B2, US20110177073A1, EP2847231A1, WO2016079081A1, and EP3055329A 1, the contents of each of which is incorporated by reference herein.

### CrossMab

Another option to reduce light chain mispairing is the CrossMab technology which avoids non-specific L chain mispairing by exchanging CH1 and CL domains in the Fab of one half of the bispecific antibody. Such crossover variants retain binding specificity and affinity, but make the two arms so different that L chain mispairing is prevented. The CrossMab technology (as reviewed in Klein et al. Supra) involves domain swapping between heavy and light chains so as to promote the formation of the correct pairings. Briefly, to construct a bispecific IgG-like CrossMab antibody that could bind to two antigens by using two distinct light chain-heavy chain pairs, a two-step modification process is applied. First, a dimerization interface is engineered into the C-terminus of each heavy chain using a heterodimerization approach, e.g., Knob-into-hole (KiH) technology, to ensure that only a heterodimer of two distinct heavy chains from one antibody (e.g., Antibody A) and a second antibody (e.g., Antibody B) is efficiently formed. Next, the constant heavy 1 (CH1) and constant light (CL) domains of one antibody are exchanged (Antibody A), keeping the variable heavy (VH) and variable light (VL) domains consistent. The exchange of the CH1 and CL domains ensured that the modified antibody (Antibody A) light chain would only efficiently dimerize with the modified antibody (antibody A) heavy chain, while the unmodified antibody (Antibody B) light chain would only efficiently dimerize with the unmodified antibody (Antibody B) heavy chain; and thus only the desired bispecific CrossMab would be efficiently formed (see e.g., Cain, C. SciBX 4(28); doi:10.1038/scibx.2011.783, the contents of which are incorporated by reference herein).

### Common Heavy Chain

An exemplary method of enhancing the formation of a desired bispecific antibody from a mixture of monomers is by providing a common variable heavy chain to interact with each of the heteromeric variable light chain regions of the bispecific antibody. Compositions and methods of producing bispecific antibodies with a common heavy chain are disclosed in, e.g., US20120184716, US20130317200, and US20160264685A 1, the contents of each of which is incorporated by reference herein.

### Amino Acid Modifications

Alternative compositions and methods of producing multifunctional antibodies with correct light chain pairing include various amino acid modifications. For example, Zymeworks describes heterodimers with one or more amino acid modifications in the CH1 and/or CL domains, one or more amino acid modifications in the VH and/or VL domains, or a combination thereof, which are part of the interface between the light chain and heavy chain and create preferential pairing between each heavy chain and a desired light chain such that when the two heavy chains and two light chains of the heterodimer pair are co-expressed in a cell, the heavy chain of the first heterodimer preferentially pairs with one of the light chains rather than the other (see e.g., WO2015181805). Other exemplary methods are described in WO2016026943 (Argen-X), US20150211001, US2014007258IA 1, US20160039947A1, and US20150368352.

### Lambda/Kappa Formats

Multifunctional molecules (e.g., multispecific antibody molecules) that include the lambda light chain polypeptide and a kappa light chain polypeptides, can be used to allow for heterodimerization. Methods for generating bispecific antibody molecules comprising the lambda light chain polypeptide and a kappa light chain polypeptides are disclosed in PCT/US17/53053 filed on Sep. 22, 2017 and designated publication number WO 2018/057955, incorporated herein by reference in its entirety.

In some embodiments, the multifunctional molecule includes a multispecific antibody molecule, e.g., an antibody molecule comprising two binding specificities, e.g., a bispecific antibody molecule. The multispecific antibody molecule includes:
a lambda light chain polypeptide 1 (LLCP1) specific for a first epitope,
a heavy chain polypeptide 1 (HCP1) specific for the first epitope;
a kappa light chain polypeptide 2 (KLCP2) specific for a second epitope; and
a heavy chain polypeptide 2 (HCP2) specific for the second epitope.

“Lambda light chain polypeptide 1 (LLCP1)”, as that term is used herein, refers to a polypeptide comprising sufficient light chain (LC) sequence, such that when combined with a cognate heavy chain variable region, can mediate specific binding to its epitope and complex with an HCP1. In some embodiments, it comprises all or a fragment of a CH1 region. In some embodiments, an LLCP1 comprises LC-CDR1, LC-CDR2, LC-CDR3, FR1, FR2, FR3. FR4, and CH1, or sufficient sequence therefrom to mediate specific binding of its epitope and complex with an HCP1. LLCP1, together with its HCP1, provide specificity for a first epitope (while KLCP2, together with its HCP2, provide specificity for a second epitope). As described elsewhere herein, LLCP1 has a higher affinity for HCP1 than for HCP2.

“Kappa light chain polypeptide 2 (KLCP2)”, as that term is used herein, refers to a polypeptide comprising sufficient light chain (LC) sequence, such that when combined with a cognate heavy chain variable region, can mediate specific binding to its epitope and complex with an HCP2. In some embodiments, it comprises all or a fragment of a CH1 region. In some embodiments, a KLCP2 comprises LC-CDR1, LC-CDR2, LC-CDR3, FR1, FR2, FR3. FR4, and CH1, or sufficient sequence therefrom to mediate specific binding of its epitope and complex with an HCP2. KLCP2, together with its HCP2, provide specificity for a second epitope (while LLCP1, together with its HCP1, provide specificity for a first epitope).

“Heavy chain polypeptide 1 (HCP1)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In some embodiments, it comprises all or a fragment of a CH1region. In some embodiments, it comprises all or a fragment of a CH2 and/or CH3 region. In some embodiments, an HCP1 comprises HC-CDR 1, HC-CDR2, HC-CDR3, FR1, FR2, FR3, FR4, CH1, CH2, and CH3, or sufficient sequence therefrom to: (i) mediate specific binding of its epitope and complex with an LLCP1, (ii) to complex preferentially, as described herein to LLCP1 as opposed to KLCP2; and (iii) to complex preferentially, as described herein, to an HCP2, as opposed to another molecule of HCP1. HCP1, together with its LLCP1, provide specificity for a first epitope (while KLCP2, together with its HCP2, provide specificity for a second epitope).

“Heavy chain polypeptide 2 (HCP2)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In some embodiments, it comprises all or a fragment of a CH1 region. In some embodiments, it comprises all or a fragment of a CH2 and/or CH3 region. In some embodiments, an HCP1 comprises HC-CDR1, HC-CDR2, HC-CDR3, FR1, FR2, FR3, FR4, CH1. CH2, and CH3, or sufficient sequence therefrom to: (i) mediate specific binding of its epitope and complex with an KLCP2, (ii) to complex preferentially, as described herein to KLCP2 as opposed to LLCP1; and (iii) to complex preferentially, as described herein, to an HCP1, as opposed to another molecule of HCP2. HCP2, together with its KLCP2, provide specificity for a second epitope (while LLCP1, together with its HCP1, provide specificity for a first epitope).

In some embodiments, in the multifunctional polypeptide molecule as described herein:
LLCP1 has a higher affinity for HCP1 than for HCP2; and/or
KLCP2 has a higher affinity for HCP2 than for HCP1.

In some embodiments, the affinity of LLCP1 for HCP1 is sufficiently greater than its affinity for HCP2, such that under preselected conditions, e.g., in aqueous buffer, e.g., at pH 7, in saline, e.g., at pH 7, or under physiological conditions, at least 75, 80, 90, 95, 98, 99, 99.5, or 99.9% of the multispecific antibody molecule molecules have a LLCP1 complexed, or interfaced with, a HCP1.

In some embodiments, in the multifunctional polypeptide molecule as described herein: the HCP1 has a greater affinity for HCP2, than for a second molecule of HCP1; and/or the HCP2 has a greater affinity for HCP1, than for a second molecule of HCP2.

In some embodiments, the affinity of HCP1 for HCP2 is sufficiently greater than its affinity for a second molecule of HCP1, such that under preselected conditions, e.g., in aqueous buffer, e.g., at pH 7, in saline, e.g., at pH 7, or under physiological conditions, at least 75%, 80, 90, 95, 98, 99 99.5 or 99.9% of the multifunctional antibody molecule molecules have a HCP1 complexed, or interfaced with, a HCP2.

In another aspect, described herein is a method for making, or producing, a multifunctional antibody molecule. The method includes:
(i) providing a first heavy chain polypeptide (e.g., a heavy chain polypeptide comprising one, two, three or all of a first heavy chain variable region (first VH), a first CH1, a first heavy chain constant region (e.g., a first CH2, a first CH3, or both));
(ii) providing a second heavy chain polypeptide (e.g., a heavy chain polypeptide comprising one, two, three or all of a second heavy chain variable region (second VH), a second CH1, a second heavy chain constant region (e.g., a second CH2, a second CH3, or both));
(iii) providing a lambda chain polypeptide (e.g., a lambda light variable region (VLλ), a lambda light constant chain (VLλ), or both) that preferentially associates with the first heavy chain polypeptide (e.g., the first VH); and
(iv) providing a kappa chain polypeptide (e.g., a lambda light variable region (VLλ), a lambda light constant chain (VLλ), or both) that preferentially associates with the second heavy chain polypeptide (e.g., the second VH), under conditions where (i)-(iv) associate.

In some embodiments, the first and second heavy chain polypeptides form an Fc interface that enhances heterodimerization.

In some embodiments, (i)-(iv) (e.g., nucleic acid encoding (i)-(iv)) are introduced in a single cell, e.g., a single mammalian cell, e.g., a CHO cell. In some embodiments, (i)-(iv) are expressed in the cell. In some embodiments, (i)-(iv) (e.g., nucleic acid encoding (i)-(iv)) are introduced in different cells, e.g., different mammalian cells, e.g., two or more CHO cell. In some embodiments, (i)-(iv) are expressed in the cells.

In some embodiments, the method further comprises purifying a cell-expressed antibody molecule, e.g., using a lambda- and/or kappa-specific purification, e.g., affinity chromatography.

In some embodiments, the method further comprises evaluating the cell-expressed multifunctional antibody molecule. For example, the purified cell-expressed multifunctional antibody molecule can be analyzed by techniques known in the art, include mass spectrometry. In some embodiments, the purified cell-expressed antibody molecule is cleaved, e.g., digested with papain to yield the Fab moieties and evaluated using mass spectrometry.

In some embodiments, the method produces correctly paired kappa/lambda multispecific, e.g., bispecific, antibody molecules in a high yield, e.g., at least 75%, 80, 90, 95, 98, 99 99.5 or 99.9%,

In other embodiments, the multispecific, e.g., a bispecific, antibody molecule that includes:
(i) a first heavy chain polypeptide (HCP1) (e.g., a heavy chain polypeptide comprising one, two, three or all of a first heavy chain variable region (first VH), a first CH1, a first heavy chain constant region (e.g., a first CH2, a first CH3, or both)), e.g., wherein the HCP1 binds to a first epitope;
(ii) a second heavy chain polypeptide (HCP2) (e.g., a heavy chain polypeptide comprising one, two, three or all of a second heavy chain variable region (second VH), a second CH1, a second heavy chain constant region (e.g., a second CH2, a second CH3, or both)), e.g., wherein the HCP2 binds to a second epitope;
(iii) a lambda light chain polypeptide (LLCP1) (e.g., a lambda light variable region (VLλ), a lambda light constant chain (VLλ), or both) that preferentially associates with the first heavy chain polypeptide (e.g., the first VH), e.g., wherein the LLCP1 binds to a first epitope; and
(iv) a kappa light chain polypeptide (KLCP2) (e.g., a kappa light variable region (VLκ), a kappa light constant chain (VLκ), or both) that preferentially associates with the second heavy chain polypeptide (e.g., the second VH), e.g., wherein the KLCP2 binds to a second epitope.

In some embodiments, the first and second heavy chain polypeptides form an Fc interface that enhances heterodimerization. In some embodiments, the multifunctional antibody molecule has a first binding specificity that includes a hybrid VLλ-CLλ heterodimerized to a first heavy chain variable region connected to the Fc constant, CH2-CH3 domain (having a knob modification) and a second binding specificity that includes a hybrid VLκ-CLκ heterodimerized to a second heavy chain variable region connected to the Fc constant, CH2-CH3 domain (having a hole modification).

### Multispecific or Multifunctional Antibody Molecules

Exemplary structures of multispecific and multifunctional molecules defined herein are described throughout. Exemplary structures are further described in: Weidle U et al. (2013) The Intriguing Options of Multispecific Antibody Formats for Treatment of Cancer. Cancer Genomics & Proteomics 10: 1-18 (2013); and Spiess C et al. (2015) Alternative molecular formats and therapeutic applications for bispecific antibodies. Molecular Immunology 67: 95-106; the full contents of each of which is incorporated by reference herein).

In some embodiments, multispecific antibody molecules can comprise more than one antigen-binding site, where different sites are specific for different antigens. In some embodiments, multispecific antibody molecules can bind more than one (e.g., two or more) epitopes on the same antigen. In some embodiments, multispecific antibody molecules comprise an antigen-binding site specific for a target cell (e.g., cancer cell) and a different antigen-binding site specific for an immune effector cell. In some embodiments, the multispecific antibody molecule is a bispecific antibody molecule. Bispecific antibody molecules can be classified into five different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates.

BsIgG is a format that is monovalent for each antigen. Exemplary BsIgG formats include but are not limited to crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, ick-body, orthogonal Fab. See Spiess et al. Mol. Immunol. 67(2015):95-106. Exemplary BsIgGs include catumaxomab (Fresenius Biotech, Trion Pharma, Neopharm), which contains an anti-CD3 arm and an anti-EpCAM arm; and ertumaxomab (Neovii Biotech, Fresenius Biotech), which targets CD3 and HER2. In some embodiments, BsIgG comprises heavy chains that are engineered for heterodimerization. For example, heavy chains can be engineered for heterodimerization using a “knobs-into-holes” strategy, a SEED platform, a common heavy chain (e.g., in ck-bodies), and use of heterodimeric Fc regions. See Spiess et al. Mol. Immunol. 67(2015):95-106. Strategies that have been used to avoid heavy chain pairing of homodimers in BsIgG include knobs-in-holes, duobody, azymetric, charge pair, HA-TF, SEEDbody, and differential protein A affinity. Se Id. BsIgG can be produced by separate expression of the component antibodies in different host cells and subsequent purification/assembly into a BsIgG. BsIgG can also be produced by expression of the component antibodies in a single host cell. BsIgG can be purified using affinity chromatography, e.g., using protein A and sequential pH elution.

IgG appended with an additional antigen-binding moiety is another format of bispecific antibody molecules. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). See Id Examples of appended IgG formats include dual variable domain IgG (DVD-Ig), IgG(I)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. An example of an IgG-scFv is MM-141 (Merrimack Pharmaceuticals), which binds IGF-1R and HER3. Examples of DVD-Ig include ABT-981 (AbbVie), which binds IL-1α and IL-1β; and ABT-122 (AbbVie), which binds TNF and IL-17A.

Bispecific antibody fragments (BsAb) are a format of bispecific antibody molecules that lack some or all of the antibody constant domains. For example, some BsAb lack an Fc region. In some embodiments, bispecific antibody fragments include heavy and light chain regions that are connected by a peptide linker that permits efficient expression of the BsAb in a single host cell. Exemplary bispecific antibody fragments include but are not limited to nanobody, nanobody-HAS, BiTE, Diabody, DART, TandAb, scDiabody, scDiabody-CH3. Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fe, tandem scFv-Fc, and intrabody. See Id. For example, the BiTE format comprises tandem scFvs, where the component scFvs bind to CD3 on T cells and a surface antigen on cancer cells.

Bispecific fusion proteins include antibody fragments linked to other proteins, e.g., to add additional specificity and/or functionality. An example of a bispecific fusion protein is an immTAC, which comprises an anti-CD3 scFv linked to an affinity-matured T-cell receptor that recognizes HLA-presented peptides. In some embodiments, the dock-and-lock (DNL) method can be used to generate bispecific antibody molecules with higher valency. Also, fusions to albumin binding proteins or human serum albumin can be extend the serum half-life of antibody fragments. See Id.

In some embodiments, chemical conjugation, e.g., chemical conjugation of antibodies and/or antibody fragments, can be used to create BsAb molecules. See Id. An exemplary bispecific antibody conjugate includes the CovX-body format, in which a low molecular weight drug is conjugated site-specifically to a single reactive lysine in each Fab arm or an antibody or fragment thereof. In some embodiments, the conjugation improves the serum half-life of the low molecular weight drug. An exemplary CovX-body is CVX-241 (NCT01004822), which comprises an antibody conjugated to two short peptides inhibiting either VEGF or Ang2. Se Id.

The antibody molecules can be produced by recombinant expression, e.g., of at least one or more component, in a host system. Exemplary host systems include eukaryotic cells (e.g., mammalian cells, e.g., CHO cells, or insect cells, e.g., SF9 or S2 cells) and prokaryotic cells (e.g., E. coli). Bispecific antibody molecules can be produced by separate expression of the components in different host cells and subsequent purification/assembly. Alternatively, the antibody molecules can be produced by expression of the components in a single host cell. Purification of bispecific antibody molecules can be performed by various methods such as affinity chromatography, e.g., using protein A and sequential pH elution. In other embodiments, affinity tags can be used for purification, e.g., histidine-containing tag, myc tag, or streptavidin tag.

### Linkers

The multispecific or multifunctional molecule as described herein can further include a linker, e.g., a linker between one or more of: the antigen binding domain and the cytokine molecule, the antigen binding domain and the immune cell engager, the antigen binding domain and the stromal modifying moiety, the cytokine molecule and the immune cell engager, the cytokine molecule and the stromal modifying moiety, the immune cell engager and the stromal modifying moiety, the antigen binding domain and the immunoglobulin chain constant region, the cytokine molecule and the immunoglobulin chain constant region, the immune cell engager and the immunoglobulin chain constant region, or the stromal modifying moiety and the immunoglobulin chain constant region. In some embodiments, the linker is chosen from: a cleavable linker, a non-cleavable linker, a peptide linker, a flexible linker, a rigid linker, a helical linker, or a non-helical linker, or a combination thereof.

In some embodiments, the multispecific molecule can include one, two, three or four linkers, e.g., a peptide linker. In some embodiments, the peptide linker includes Gly and Ser. In some embodiments, the peptide linker is selected from GGGGS (SEQ ID NO: 3307); GGGGSGGGGS (SEQ ID NO: 3308); GGGGSGGGGSGGGGS (SEQ ID NO: 3309): DVPSGPGGGGGSGGGGS (SEQ ID NO: 3310); and GGGGSGGGGSGGGGGS (SEQ ID NO: 3643). In some embodiments, the peptide linker is a A(EAAAK)nA (SEQ ID NO: 3437) family of linkers (e.g., as described in Protein Eng. (2001) 14 (8): 529-532). These are stiff helical linkers with n ranging from 2-5. In some embodiments, the peptide linker is selected from AEAAAKEAAAKAAA (SEQ ID NO: 3314); AEAAAKEAAAKEAAAKAAA (SEQ ID NO: 3315): AEAAAKEAAAKEAAAKEAAAKAAA (SEQ ID NO: 3316); and AEAAAKEAAAKEAAAKEAAAKEAAAKAAA(SEQ ID NO: 3317).

### Nucleic Acids

Described herein, in certain embodiments, is an isolated nucleic acid molecule comprising a nucleotide sequence having at least 75%, 80%, 85%, 90%, 95%, 99%, 99.5%, 99.9%, or 100% sequence identity to the nucleotide sequence encoding the multifunctional polypeptide molecule as described herein.

Nucleic acids encoding the aforementioned antibody molecules, e.g., anti-TCRβV antibody molecules, multispecific or multifunctional molecules are also disclosed.

In certain embodiments, the invention features nucleic acids comprising nucleotide sequences that encode heavy and light chain variable regions and CDRs or hypervariable loops of the antibody molecules, as described herein. For example, the invention features a first and second nucleic acid encoding heavy and light chain variable regions, respectively, of an antibody molecule chosen from one or more of the antibody molecules as described herein. The nucleic acid can comprise a nucleotide sequence as set forth in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein.

In certain embodiments, the nucleic acid can comprise a nucleotide sequence encoding at least one, two, or three CDRs or hypervariable loops from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In other embodiments, the nucleic acid can comprise a nucleotide sequence encoding at least one, two, or three CDRs or hypervariable loops from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In yet another embodiment, the nucleic acid can comprise a nucleotide sequence encoding at least one, two, three, four, five, or six CDRs or hypervariable loops from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs or hypervariable loops from a heavy chain variable region having the nucleotide sequence as set forth in the tables herein, a sequence substantially homologous thereto (e.g., a sequence at least about 85%, 90%, 95%, 99% or more identical thereto, and/or capable of hybridizing under the stringency conditions described herein). In another embodiment, the nucleic acid can comprise a nucleotide sequence encoding at least one, two, or three CDRs or hypervariable loops from a light chain variable region having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein). In yet another embodiment, the nucleic acid can comprise a nucleotide sequence encoding at least one, two, three, four, five, or six CDRs or hypervariable loops from heavy and light chain variable regions having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein).

In certain embodiments, the nucleic acid can comprise a nucleotide sequence encoding a cytokine molecule, an immune cell engager, or a stromal modifying moiety as described herein.

In another aspect, the application features host cells and vectors containing the nucleic acids described herein. The nucleic acids may be present in a single vector or separate vectors present in the same host cell or separate host cell, as described in more detail hereinbelow.

### Vectors

Described herein, in certain embodiments, is a vector comprising one or more of the nucleic acid molecules as described herein.

Further provided herein are vectors comprising the nucleotide sequences encoding antibody molecules, e.g., anti-TCRβV antibody molecules, or a multispecific or multifunctional molecule described herein. In some embodiments, the vectors comprise nucleic acid sequences encoding antibody molecules, e.g., anti-TCRβV antibody molecules, or multispecific or multifunctional molecule described herein. In some embodiments, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

Described herein, in certain embodiments, is a cell comprising the nucleic acid as described herein or the vector as described herein.

In another aspect, described herein are host cells and vectors containing the nucleic acids. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell.

In some embodiments, described herein are host cells comprising a nucleic acid encoding an antibody molecule as described herein.

In some embodiments, described herein are the host cells genetically engineered to comprise nucleic acids encoding the antibody molecule.

In some embodiments, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

In some embodiments, described herein are host cells comprising the vectors described herein. The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

Method of Expanding Cells with Anti-TCRVB Antibodies

Any of the compositions and methods described herein can be used to expand an immune cell population. An immune cell provided herein includes an immune cell derived from a hematopoietic stem cell or an immune cell derived from a non-hematopoietic stem cell, e.g., by differentiation or de-differentiation.

An immune cell includes a hematopoietic stem cell, progeny thereof and/or cells that have differentiated from said HSC, e.g., lymphoid cells or myeloid cells. An immune cell can be an adaptive immune cell or an innate immune cell. Examples of immune cells include T cells, B cells, Natural Killer cells, Natural Killer T cells, neutrophils, dendritic cells, monocytes, macrophages, and granulocytes.

In some embodiments, an immune cell is a T cell. In some embodiments, a T cell includes a CD4+ T cell, a CD8+ T cell, a TCR alpha-beta T cell, a TCR gamma-delta T cell. In some embodiments, a T cell comprises a memory T cell (e.g., a central memory T cell, or an effector memory T cell (e.g., a TEMRA) or an effector T cell. In some embodiments, a T cell comprises a tumor infiltrating lymphocyte (TIL).

In some embodiments, an immune cell is an NK cell.

In some embodiments, an immune cell is a TIL. TILs are immune cells (e.g., T cells, B cells or NK cells) that can be found in a tumor or around a tumor (e.g., in the stroma or tumor microenvironment of a tumor), e.g., a solid tumor, e.g., as described herein. TILs can be obtained from a sample from a subject having cancer, e.g., a biopsy or a surgical sample. In some embodiments, TILs can be expanded using a method as described herein. In some embodiments, a population of expanded TILs, e.g., expanded using a method as described herein, can be administered to a subject to treat a disease, e.g., a cancer.

In some embodiments, immune cells, e.g., T cells (e.g., TILs), can be obtained from a unit of blood collected from a subject using any number of techniques known to the skilled artisan, such as Ficoll™ separation. In one aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In some embodiments, the cells are washed with phosphate buffered saline (PBS). In an alternative embodiment, the wash solution lacks calcium and may lack magnesium or may lack many if not all divalent cations. The methods described herein can include more than one selection step, e.g., more than one depletion step.

In some embodiments, the methods of the application can utilize culture media conditions comprising DMEM, DMEM F12, RPMI 1640, and/or AIM V media. The media can be supplemented with glutamine. HEPES buffer (e.g., 10 mM), serum (e.g., heat-inactivated serum, e.g., 10%), and/or beta mercaptoethanol (e.g., 55 uM). IN some embodiments, the culture conditions as described herein comprise one or more supplements, cytokines, growth factors, or hormones. In some embodiments, the culture condition comprises one or more of IL-2, IL-15, or IL-7, or a combination thereof.

Immune effector cells such as T cells may be activated and expanded generally using methods as described, for example, in U.S. Pat. Nos. 6,352,694; 6,534,055; or 6,905,680. Generally, a population of immune cells, may be expanded by contact with an agent that stimulates a CD3/TCR complex associated signal and a ligand that stimulates a costimulatory molecule on the surface of the T cells; and/or by contact with a cytokine, e.g., IL-2, IL-15 or IL-7. T cell expansion protocols can also include stimulation, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3. B-T3. XR-CD28 (Diaclone, Besangon, France) can be used as can other methods commonly known in the art (Berg et al., Transplant Proc. 30(8):3975-3977, 1998: Haanen et al., J. Exp. Med. 190(9):13191328, 1999; Garland et al., J. Immunol Meth. 227(1-2):53-63, 1999).

A TIL population can also be expanded by methods known in the art. For example, a population of TILs can be expanded as described in Hall et al., Journal for Immunol Therapy of Cancer (2016) 4:61, the entire contents of which are hereby incorporated by reference. Briefly. TILs can be isolated from a sample by mechanical and/or physical digestion. The resultant TIL population can be stimulated with an anti-CD3 antibody in the presence of non-dividing feeder cells. In some embodiments, the TIL population can be cultured, e.g., expanded, in the presence of IL-2, e.g., human IL-2. In some embodiments, the TIL cells can be cultured, e.g., expanded for a period of at least 1-21 days, e.g., at least 1, 2, 3, 4, 5, 6, 7, 8, 9, 10, 11, 12, 13, 14, 15, 16, 17, 18, 19, 20 or 21 days.

As described herein, in some embodiments, an immune cell population (e.g., a T cell (e.g., a T_{EMRA} cell or a TIL population) can be expanded by contacting the immune cell population with an anti-TCRβVB antibody, e.g., as described herein.

In some embodiments, the expansion occurs in vivo, e.g., in a subject. In some embodiments, a subject is administered the multispecific or multifunctional molecules comprising TCRβV-binding moieties as described herein resulting in expansion of immune cells in vivo.

In some embodiments, the expansion occurs ex vivo, e.g., in vitro. In some embodiments, cells from a subject, e.g., T cells, e.g., TIL cells, are expanded in vitro with the multispecific or multifunctional molecules as described herein. In some embodiments, the expanded TILs are administered to the subject to treat a disease or a symptom of a disease.

In some embodiments, a method of expansion as described herein results in an expansion of at least 1.1-10 fold, 10-20 fold, or 20-50 fold expansion. In some embodiments, the expansion is at least 1.1. 1.2, 1.3, 1.4, 1.5, 2, 3, 4, 5, 6, 7, 8, 9, 10, 15, 20, 25, 30, 35, 40, 45 or 50 fold expansion.

In some embodiments, a method of expansion as described herein comprises culturing. e.g., expanding, the cells for at least about 4 hours, 6 hours, 10 hours, 12 hours, 15 hours, 18 hours, 20 hours, or 22 hours. In some embodiments, a method of expansion as described herein comprises culturing, e.g., expanding, the cells for at least 1, 2, 3, 4, 5, 6, 7, 8, 9, 10, 11, 12, 13, 14, 15, 16, 17, 18, 19, 20 or 21 days. In some embodiments, a method of expansion as described herein comprises culturing, e.g., expanding, the cells for at least about 1 week, 2 weeks, 3 weeks, 4 weeks, 5 weeks, 6 weeks, 7 weeks or 8 weeks.

In some embodiments, a method of expansion as described herein is performed on immune cells obtained from a healthy subject.

In some embodiments, a method of expansion as described herein is performed on immune cells (e.g., TILs) obtained from a subject having a disease. e.g., a cancer, e.g., a solid tumor as described herein.

In some embodiments, a method of expansion as described herein further comprises contacting the population of cells with an agent, that promotes, e.g., increases, immune cell expansion. In some embodiments, the agent comprises an immune checkpoint inhibitor. e.g., a PD-1 inhibitor, a LAG-3 inhibitor, a CTLA4 inhibitor, or a TIM-3 inhibitor. In some embodiments, the agent comprises a 4-1BB agonist, e.g., an anti-4-1BB antibody.

Without wishing to be bound by theory, in some embodiments, the multispecific or multifunctional molecules as described herein can expand, e.g., selectively or preferentially expand, T cells expressing a T cell receptor (TCR) comprising a TCR alpha and/or TCR beta molecule, e.g., TCR alpha-beta T cells (αβ T cells). In some embodiments, the multispecific or multifunctional molecules as described herein do not expand, or induce proliferation of T cells expressing a TCR comprising a TCR gamma and/or TCR delta molecule, e.g., TCR gamma-delta T cells (γδ T cells). In some embodiments, the multispecific or multifunctional molecules as described herein selectively or preferentially expand αβ T cells over γδ T cells.

Without wishing to be bound by theory, it is believed that, in some embodiments, γδ T cells are associated with cytokine release syndrome (CRS) and/or neurotoxicity (NT). In some embodiments, the multispecific or multifunctional molecules as described herein result in selective expansion of non-γδ T cells, e.g., expansion of ai T cells, thus reducing CRS and/or NT.

In some embodiments, any of the compositions or methods as described herein result in an immune cell population having a reduction of, e.g., depletion of, γδ T cells. In some embodiments, the immune cell population is contacted with an agent that reduces, e.g., inhibits or depletes, γδ T cells, e.g., an anti-IL-17 antibody or an agent that binds to a TCR gamma and/or TCR delta molecule.

### CRS Grading

In some embodiments, CRS (Cytokine Release Syndrome) can be graded in severity from 1-5 as follows. Grades 1-3 are less than severe CRS. Grades 4-5 are severe CRS. For Grade 1 CRS, only symptomatic treatment is needed (e.g., nausea, fever, fatigue, myalgias, malaise, headache) and symptoms are not life threatening. For Grade 2 CRS, the symptoms require moderate intervention and generally respond to moderate intervention. Subjects having Grade 2 CRS develop hypotension that is responsive to either fluids or one low-dose vasopressor; or they develop grade 2 organ toxicity or mild respiratory symptoms that are responsive to low flow oxygen (<40% oxygen). In Grade 3 CRS subjects, hypotension generally cannot be reversed by fluid therapy or one low-dose vasopressor. These subjects generally require more than low flow oxygen and have grade 3 organ toxicity (e.g., renal or cardiac dysfunction or coagulopathy) and/or grade 4 transaminitis. Grade 3 CRS subjects require more aggressive intervention, e.g., oxygen of 40% or higher, high dose vasopressor(s), and/or multiple vasopressors. Grade 4 CRS subjects suffer from immediately life-threatening symptoms, including grade 4 organ toxicity or a need for mechanical ventilation. Grade 4 CRS subjects generally do not have transaminitis. In Grade 5 CRS subjects, the toxicity causes death. Sets of criteria for grading CRS are provided herein as Table 5, Table 6, and Table 7. Unless otherwise specified, CRS as used herein refers to CRS according to the criteria of Table 6.

In some embodiments, CRS is graded according to Table 5.

The term “cytokine profile” as used herein, refers to the level and/or activity of on one or more cytokines or chemokines, e.g., as described herein. In some embodiments, a cytokine profile comprises the level and/or activity of a naturally occurring cytokine, a fragment or a functional fragment or a functional variant thereof. In some embodiments, a cytokine profile comprises the level and/or activity of one or more cytokines and/or one or more chemokines (e.g., as described herein). In some embodiments, a cytokine profile comprises the level and/or activity of a naturally occurring cytokine, a fragment or a functional fragment or a functional variant thereof. In some embodiments, a cytokine profile comprises the level and/or activity of a naturally occurring chemokine, a fragment or a functional fragment or a functional variant thereof. In some embodiments, a cytokine profile comprises the level and/or activity of one or more of: IL-2 (e.g., full length, a variant, or a fragment thereof); IL-1beta (e.g., full length, a variant, or a fragment thereof); IL-6 (e.g., full length, a variant, or a fragment thereof); TNFα (e.g., full length, a variant, or a fragment thereof): IFNgamma (e.g., full length, a variant, or a fragment thereof) IL-10 (e.g., full length, a variant, or a fragment thereof); IL-4 (e.g., full length, a variant, or a fragment thereof); TNF alpha (e.g., full length, a variant, or a fragment thereof); IL-12p70 (e.g., full length, a variant, or a fragment thereof); IL-13 (e.g., full length, a variant, or a fragment thereof); IL-8 (e.g., full length, a variant, or a fragment thereof); Eotaxin (e.g., full length, a variant, or a fragment thereof); Eotaxin-3 (e.g., full length, a variant, or a fragment thereof): IL-8 (HA) (e.g., full length, a variant, or a fragment thereof); IP-10 (e.g., full length, a variant, or a fragment thereof); MCP-1 (e.g., full length, a variant, or a fragment thereof); MCP-4 (e.g., full length, a variant, or a fragment thereof); MDC (e.g., full length, a variant, or a fragment thereof); MIP-1a (e.g., full length, a variant, or a fragment thereof); MIP-1b (e.g., full length, a variant, or a fragment thereof); TARC (e.g., full length, a variant, or a fragment thereof); GM-CSF (e.g., full length, a variant, or a fragment thereof); IL-12 23p40 (e.g., full length, a variant, or a fragment thereof); IL-15 (e.g., full length, a variant, or a fragment thereof); IL-16 (e.g., full length, a variant, or a fragment thereof); IL-17a (e.g., full length, a variant, or a fragment thereof); IL-1a (e.g., full length, a variant, or a fragment thereof): IL-5 (e.g., full length, a variant, or a fragment thereof); IL-7 (e.g., full length, a variant, or a fragment thereof); TNF-beta (e.g., full length, a variant, or a fragment thereof); or VEGF (e.g., full length, a variant, or a fragment thereof). In some embodiments, a cytokine profile includes secretion of one or more cytokines or chemokines. In some embodiments, a cytokine in a cytokine profile can be modulated, e.g., increased or decreased, by an anti-TCRβV antibody molecule described herein. In some embodiments, the cytokine profile includes cytokines associated with a cytokine storm or cytokine release syndrome (CRS), e.g., IL-6, IL-1beta, TNFalpha and IL-10.

### Pharmaceutical Compositions

Described herein, in certain embodiments, is a pharmaceutical composition comprising the multifunctional polypeptide molecule as described herein, the nucleic acid molecules as described herein, the vector as described herein, or the cell as described herein, and a pharmaceutically acceptable carrier, excipient, or diluent.

Pharmaceutical compositions or formulations comprising the agent, e.g., the multifunctional or multispecific molecules, of the described compositions and for use in any of the described methods can be prepared according to conventional techniques well known in the pharmaceutical industry and described in the published literature. In some embodiments, a pharmaceutical composition or formulation for treating a subject comprises an effective amount of any the multifunctional or multispecific molecules or the compositions as described herein, or a pharmaceutically acceptable salt, solvate, hydrate or ester thereof. The pharmaceutical formulation comprising the multifunctional or multispecific molecules as described herein may further comprise a pharmaceutically acceptable excipient, diluent or carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66: 1-19 (1977), incorporated herein by reference for this purpose. The salts can be prepared in situ during the final isolation and purification of the compounds, or separately by reacting the free base form with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, pahnitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some embodiments, the compositions are formulated into any of many possible dosage forms such as, but not limited to, tablets, capsules, gel capsules, liquid syrups, soft gels, suppositories, and enemas. In some embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In some embodiments, a pharmaceutical formulation or composition as described herein includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In some embodiments, liposomes also include sterically stabilized liposomes. e.g., liposomes comprising one or more specialized lipids. These specialized lipids result in liposomes with enhanced circulation lifetimes. In some embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In some embodiments, the present disclosure employs a penetration enhancer to effect the efficient delivery of the multifunctional or multispecific molecules or the compositions as described herein, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

In some embodiments, the pharmaceutical formulation comprises multiple multifunctional or multispecific molecules as described herein. In some embodiments, the multifunctional or multispecific molecules or the compositions as described herein is administered in combination with another drug or therapeutic agent.

In some embodiments, the pharmaceutical formulation comprises multiple multifunctional or multispecific molecules as described herein and a pharmaceutically acceptable excipient. In some embodiments, the excipient may comprise one or more of L-histidine/L-histidine monohydrochloride buffer, sucrose, or polysorbate. The polysorbate may be polyoxyethylene (20) sorbitan monooleate (e.g., polysorbate 80), polyoxyethylene (20) sorbitan monolaurate (e.g., polysorbate 20), polyoxyethylene (20) sorbitan monopalmitate (e.g., polysorbate 40), polyoxyethylene (20) sorbitan monostearate (e.g., polysorbate 60), or a functional variant thereof. In some embodiments, the polysorbate may be polysorbate-80. In some embodiments, the pharmaceutical composition comprising the pharmaceutically acceptable excipient may be formulated as a stock solution. In some embodiments, the pharmaceutical composition comprising the pharmaceutically acceptable excipient may be formulated as a dose to administer to a subject in need thereof. A pharmaceutical composition formulated as a stock solution may be diluted to a concentration to administer to a subject in need thereof. Following mixture of a stock solution of the pharmaceutical composition, a volume (e.g., at least 1 mL, at least 2 mL, at least 3 mL, at least 4 mL, at least 5 mL, at least 8 mL, or at least 10 mL) of the stock solution may be placed in a vial for dilution. A pharmaceutical composition or formulation may be maintained in a vial for 1 day, 2 days, 3 days, 4 days, 5 days, 6 days, 7 days, 8 days, 9 days, 10 days, or more, prior to administration to a subject in need thereof. On a day of administration to a subject in need thereof, a stock solution of a pharmaceutical composition described herein may be diluted 2-fold, 3-fold, 4-fold, 5-fold, 6-fold, 7-fold, 8-fold, 9-fold, 10-fold, 12-fold, 15-fold, or 20-fold from a concentration of the stock solution. Following dilution, the pharmaceutical composition or formulation may be refrigerated. Following dilution, the pharmaceutical composition or formulation may be stored at room temperature. Following dilution, the pharmaceutical composition or formulation may be stored up to 1 hour, up to 2 hours, up to 3 hours, up to 4 hours, up to 5 hours, up to 6 hours, up to 12 hours, up to 24 hours, up to 36 hours, up to 48 hours, or up to 72 hours in a syringe or IV bag system prior to administration.

In some embodiments, the pharmaceutical composition described herein or a dose of a pharmaceutical composition described herein may comprise L-histidine/L-histidine monohydrochloride buffer, sucrose, polysorbate, or a combination thereof. In some embodiments, a stock solution of the pharmaceutical composition can be formulated to comprise a concentration of L-histidine/L-histidine monohydrochloride buffer, sucrose, polysorbate, the multifunctional molecule as described herein, or a combination thereof.

In some embodiments, a stock solution of the pharmaceutical composition or a diluted formulation of the pharmaceutical composition may comprise a concentration of L-histidine/L-histidine monohydrochloride buffer. In some embodiments, the pharmaceutical composition or formulation described herein may comprise at least about 0.1 mM, at least about 0.2 mM, at least about 0.5 mM, at least about 1 mM, at least about 2 mM, at least about 3 mM, at least about 4 mM, at least about 5 mM, at least about 10 mM, at least about 15 mM, at least about 20 mM, at least about 25 mM, at least about 30 mM, at least about 40 mM, at least about 50 mM, at least about 75 mM, at least about 100 mM, at least about 125 mM, at least about 150 mM, or at least about 200 mM L-histidine/L-histidine monohydrochloride buffer. In some embodiments, the pharmaceutical composition or formulation described herein may comprise at most about 200 mM, at most about 150 mM, at most about 125 mM, at most about 100 mM, at most about 75 mM, at most about 50 mM, at most about 40 mM, at most about 30 mM, at most about 25 mM, at most about 20 mM, at most about 15 mM, at most about 10 mM, at most about 5 mM, at most about 4 mM, at most about 3 mM, at most about 2 mM, at most about 1 mM, at most about 0.5 mM, at most about 0.2 mM, or at most about 0.1 mM L-histidine/L-histidine monohydrochloride buffer. In some embodiments, the pharmaceutical composition or formulation described herein may comprise from about 0.5 mM to about 150 mM L-histidine/L-histidine monohydrochloride buffer. In some embodiments, the pharmaceutical composition or formulation described herein may comprise from about 0.5 mM to about 1 mM, about 0.5 mM to about 2 mM, about 0.5 mM to about 5 mM, about 0.5 mM to about 10 mM, about 0.5 mM to about 15 mM, about 0.5 mM to about 20 mM, about 0.5 mM to about 25 mM, about 0.5 mM to about 50 mM, about 0.5 mM to about 75 mM, about 0.5 mM to about 100 mM, about 0.5 mM to about 150 mM, about 1 mM to about 2 mM, about 1 mM to about 5 mM, about 1 mM to about 10 mM, about 1 mM to about 15 mM, about 1 mM to about 20 mM, about 1 mM to about 25 mM, about 1 mM to about 50 mM, about 1 mM to about 75 mM, about 1 mM to about 100 mM, about 1 mM to about 150 mM, about 2 mM to about 5 mM, about 2 mM to about 10 mM, about 2 mM to about 15 mM, about 2 mM to about 20 mM, about 2 mM to about 25 mM, about 2 mM to about 50 mM, about 2 mM to about 75 mM, about 2 mM to about 100 mM, about 2 mM to about 150 mM, about 5 mM to about 10 mM, about 5 mM to about 15 mM, about 5 mM to about 20 mM, about 5 mM to about 25 mM, about 5 mM to about 50 mM, about 5 mM to about 75 mM, about 5 mM to about 100 mM, about 5 mM to about 150 mM, about 10 mM to about 15 mM, about 10 mM to about 20 mM, about 10 mM to about 25 mM, about 10 mM to about 50 mM, about 10 mM to about 75 mM, about 10 mM to about 100 mM, about 10 mM to about 150 mM, about 15 mM to about 20 mM, about 15 mM to about 25 mM, about 15 mM to about 50 mM, about 15 mM to about 75 mM, about 15 mM to about 100 mM, about 15 mM to about 150 mM, about 20 mM to about 25 mM, about 20 mM to about 50 mM, about 20 mM to about 75 mM, about 20 mM to about 100 mM, about 20 mM to about 150 mM, about 25 mM to about 50 mM, about 25 mM to about 75 mM, about 25 mM to about 100 mM, about 25 mM to about 150 mM, about 50 mM to about 75 mM, about 50 mM to about 100 mM, about 50 mM to about 150 mM, about 75 mM to about 100 mM, about 75 mM to about 150 mM, or about 100 mM to about 150 mM L-histidine/L-histidine monohydrochloride buffer.

In some embodiments, a stock solution of the pharmaceutical composition or a diluted formulation of the pharmaceutical composition may comprise a concentration of sucrose. In some embodiments, the concentration of the sucrose in the pharmaceutical composition or formulation described herein may comprise at least about 0.1% w/v, at least about 0.2% w/v, at least about 0.5% w/v, at least about 1% w/v, at least about 2% w/v, at least about 3% w/v, at least about 4% w/v, at least about 5% w/v, at least about 8% w/v, at least about 10% w/v, at least about 12% w/v, at least about 15% w/v, at least about 18% w/v, at least about 20% w/v, at least about 25% w/v, at least about 30% w/v, at least about 35% w/v, at least about 40% w/v, at least about 45% w/v, or at least about 50% w/v. In some embodiments, the concentration of the sucrose in the pharmaceutical composition or formulation described herein may comprise at most about 50% w/v, at most about 45% w/v, at most about 40% w/v, at most about 35% w/v, at most about 30% w/v, at most about 25% w/v, at most about 20% w/v, at most about 18% w/v, at most about 15% w/v, at most about 12% w/v, at most about 10% w/v, at most about 8% w/v, at most about 5% w/v, at most about 4% w/v, at most about 3% w/v, at most about 2% w/v, at most about 1% w/v, at most about 0.5% w/v, at most about 0.2% w/v, or at most about 0.1% w/v. In some embodiments, the concentration of the sucrose in the pharmaceutical composition or formulation described herein described herein may comprise from about 0.5% w/v to about 20% w/v. In some embodiments, the concentration of the sucrose in the pharmaceutical composition or formulation described herein may comprise from about 0.5% w/v to about 1% w/v, about 0.5% w/v to about 2% w/v, about 0.5% w/v to about 3% w/v, about 0.5% w/v to about 4% w/v, about 0.5% w/v to about 5% w/v, about 0.5% w/v to about 6% w/v, about 0.5% w/v to about 7% w/v, about 0.5% w/v to about 8% w/v, about 0.5% w/v to about 9% w/v, about 0.5% w/v to about 10% w/v, about 0.5% w/v to about 20% w/v, about 1% w/v to about 2% w/v, about 1% w/v to about 3% w/v, about 1% w/v to about 4% w/v, about 1% w/v to about 5% w/v, about 1% w/v to about 6% w/v, about 1% w/v to about 7% w/v, about 1% w/v to about 8% w/v, about 1% w/v to about 9% w/v, about 1% w/v to about 10% w/v, about 1% w/v to about 20% w/v, about 2% w/v to about 3% w/v, about 2% w/v to about 4% w/v, about 2% w/v to about 5% w/v, about 2% w/v to about 6% w/v, about 2% w/v to about 7% w/v, about 2% w/v to about 8% w/v, about 2% w/v to about 9% w/v, about 2% w/v to about 10% w/v, about 2% w/v to about 20% w/v, about 3% w/v to about 4% w/v, about 3% w/v to about 5% w/v, about 3% w/v to about 6% w/v, about 3% w/v to about 7% w/v, about 3% w/v to about 8% w/v, about 3% w/v to about 9% w/v, about 3% w/v to about 10% w/v, about 3% w/v to about 20% w/v, about 4% w/v to about 5% w/v, about 4% w/v to about 6% w/v, about 4% w/v to about 7% w/v, about 4% w/v to about 8% w/v, about 4% w/v to about 9% w/v, about 4% w/v to about 10% w/v, about 4% w/v to about 20% w/v, about 5% w/v to about 6% w/v, about 5% w/to about 7% w/v, about 5% w/v to about 8% w/v, about 5% w/v to about 9% w/v, about 5% w/v to about 10% w/v, about 5% w/v to about 20% w/v, about 6% w/v to about 7% w/v, about 6% w/v to about 8% w/v, about 6% w/v to about 9% w/v, about 6% w/v to about 10% w/v, about 6% w/v to about 20% w/v, about 7% w/v to about 8% w/v, about 7% w/v to about 9% w/v, about 7% w/v to about 10% w/v, about 7% w/v to about 20% w/v, about 8% w/v to about 9% w/v, about 8% w/v to about 10% w/v, about 8% w/v to about 20% w/v, about 9% w/v to about 10% w/v, about 9% w/v to about 20% w/v, or about 10% w/v to about 20% w/v.

In some embodiments, a stock solution of the pharmaceutical composition or a diluted formulation of the pharmaceutical composition may comprise a concentration of polysorbate (e.g., polysorbate-80). In some embodiments, the concentration of the polysorbate (e.g., polysorbate-80) in the pharmaceutical composition or formulation described herein may comprise at least about 0.0001% w/v, at least about 0.0005% w/v, at least about 0.001% w/v, at least about 0.005% w/v, at least about 0.01% w/v, at least about 0.02% w/v, at least about 0.03% w/v, at least about 0.04% w/v, at least about 0.05% w/v, at least about 0.1% w/v, at least about 0.2% w/v, at least about 0.3% w/v, at least about 0.4% w/v, at least about 0.5% w/v, or at least about 1% w/v. In some embodiments, the concentration of the polysorbate (e.g., polysorbate-80) in the pharmaceutical composition or formulation described herein may comprise at most about 1% w/v, at most about 0.5% w/v, at most about 0.1% w/v, at most about 0.05% w/v, at most about 0.04% w/v, at most about 0.03% w/v, at most about 0.02% w/v, at most about 0.01% w/v, at most about 0.005% w/v, at most about 0.001% w/v, at most about 0.0005% w/v, or at most about 0.0001% w/v. In some embodiments, the concentration of the polysorbate (e.g., polysorbate-80) in the pharmaceutical composition or formulation described herein may comprise from about 0.001% w/v to about 0.1% w/v. In some embodiments, the concentration of the polysorbate (e.g., polysorbate-80) in the pharmaceutical composition or formulation described herein may comprise from about 0.001% w/v to about 0.005% w/v, about 0.001% w/v to about 0.01% w/v, about 0.001% w/v to about 0.02% w/v, about 0.001% w/v to about 0.03% w/v, about 0.001% w/v to about 0.04% w/v, about 0.001% w/v to about 0.05% w/v, about 0.001% w/v to about 0.06% w/v, about 0.001% w/v to about 0.07% w/v, about 0.001% w/v to about 0.08% w/v, about 0.001% w/v to about 0.09% w/v, about 0.001% w/v to about 0.1% w/v, about 0.005% w/v to about 0.01% w/v, about 0.005% w/v to about 0.02% w/v, about 0.005% w/v to about 0.03% w/v, about 0.005% w/v to about 0.04% w/v, about 0.005% w/v to about 0.05% w/v, about 0.005% w/v to about 0.06% w/v, about 0.005% w/v to about 0.07% w/v, about 0.005% w/v to about 0.08% w/v, about 0.005% w/v to about 0.09% w/v, about 0.005% w/v to about 0.1% w/v, about 0.01% w/v to about 0.02% w/v, about 0.01% w/v to about 0.03% w/v, about 0.01% w/v to about 0.04% w/v, about 0.01% w/v to about 0.05% w/v, about 0.01% w/v to about 0.06% w/v, about 0.01% w/v to about 0.07% w/v, about 0.01% w/v to about 0.08% w/v, about 0.01% w/v to about 0.09% w/v, about 0.01% w/v to about 0.1% w/v, about 0.02% w/v to about 0.03% w/v, about 0.02% w/v to about 0.04% w/v, about 0.02% w/v to about 0.05% w/v, about 0.02% w/v to about 0.06% w/v, about 0.02% w/v to about 0.07% w/v, about 0.02% w/v to about 0.08% w/v, about 0.02% w/v to about 0.09% w/v, about 0.02% w/v to about 0.1% w/v, about 0.03% w/v to about 0.04% w/v, about 0.03% w/v to about 0.05% w/v, about 0.03% w/v to about 0.06% w/v, about 0.03% w/v to about 0.07% w/v, about 0.03% w/v to about 0.08% w/v, about 0.03% w/v to about 0.09% w/v, about 0.03% w/v to about 0.1% w/v, about 0.04% w/v to about 0.05% w/v, about 0.04% w/v to about 0.06% w/v, about 0.04% w/v to about 0.07% w/v, about 0.04% w/v to about 0.08% w/v, about 0.04% w/v to about 0.09% w/v, about 0.04% w/v to about 0.1% w/v, about 0.05% w/v to about 0.06% w/v, about 0.05% w/v to about 0.07% w/v, about 0.05% w/v to about 0.08% w/v, about 0.05% w/v to about 0.09% w/v, about 0.05% w/v to about 0.1% w/v, about 0.06% w/v to about 0.07% w/v, about 0.06% w/v to about 0.08% w/v, about 0.06% w/v to about 0.09% w/v, about 0.06% w/v to about 0.1% w/v, about 0.07% w/v to about 0.08% w/v, about 0.07% w/v to about 0.09% w/v, about 0.07% w/v to about 0.1% w/v, about 0.08% w/v to about 0.09% w/v, about 0.08% w/v to about 0.1% w/v, or about 0.09% w/v to about 0.1% w/v.

In some embodiments, a stock solution of the pharmaceutical composition or a diluted formulation of the pharmaceutical composition may comprise a concentration of a multifunctional molecule described herein. In some embodiments, the pharmaceutical composition or formulation comprising a pharmaceutically acceptable excipient described herein may comprise the multifunctional molecule described herein at a concentration of at least about at least about 0.01 mg/mL, at least about 0.05 mg/mL, at least about 0.1 mg/mL, at least about 0.5 mg/mL, at least about 1 mg/mL, at least about 2 mg/mL, at least about 3 mg/mL, at least about 4 mg/mL, at least about 5 mg/mL, at least about 10 mg/mL, at least about 15 mg/mL, at least about 20 mg/mL, at least about 25 mg/mL, at least about 30 mg/mL, at least about 40 mg/mL, at least about 50 mg/mL, at least about 75 mg/mL, at least about 100 mg/mL, at least about 125 mg/mL, at least about 150 mg/mL, at least about 200 mg/mL, at least about 250 mg/mL, or at least about 300 mg/mL. In some embodiments, the pharmaceutical composition or formulation comprising a pharmaceutically acceptable excipient described herein may comprise the multifunctional molecule described herein at a concentration of at most about 300 mg/mL, at most about 250 mg/mL, at most about 200 mg/mL, at most about 150 mg/mL, at most about 125 mg/mL, at most about 100 mg/mL, at most about 75 mg/mL, at most about 50 mg/mL, at most about 40 mg/mL, at most about 30 mg/mL, at most about 25 mg/mL, at most about 20 mg/mL, at most about 15 mg/mL, at most about 10 mg/mL, at most about 5 mg/mL, at most about 4 mg/mL, at most about 3 mg/mL, at most about 2 mg/mL, at most about 1 mg/mL, at most about 0.5 mg/mL, at most about 0.1 mg/mL, at most about 0.05 mg/mL, or at most about 0.01 mg/mL. In some embodiments, the pharmaceutical composition or formulation comprising a pharmaceutically acceptable excipient described herein may comprise the multifunctional molecule described herein at a concentration from about 0.01 mg/mL to about 50 mg/mL. In some embodiments, the pharmaceutical composition or formulation comprising a pharmaceutically acceptable excipient described herein may comprise the multifunctional molecule described herein at a concentration from about 0.01 mg/mL to about 0.05 mg/mL, about 0.01 mg/mL to about 0.1 mg/mL, about 0.01 mg/mL to about 0.5 mg/mL, about 0.01 mg/mL to about 1 mg/mL, about 0.01 mg/mL to about 3 mg/mL, about 0.01 mg/mL to about 5 mg/mL, about 0.01 mg/mL to about 10 mg/mL, about 0.01 mg/mL to about 15 mg/mL, about 0.01 mg/mL to about 20 mg/mL, about 0.01 mg/mL to about 25 mg/mL, about 0.01 mg/mL to about 50 mg/mL, about 0.05 mg/mL to about 0.1 mg/mL, about 0.05 mg/mL to about 0.5 mg/mL, about 0.05 mg/mL to about 1 mg/mL, about 0.05 mg/mL to about 3 mg/mL, about 0.05 mg/mL to about 5 mg/mL, about 0.05 mg/mL to about 10 mg/mL, about 0.05 mg/mL to about 15 mg/mL, about 0.05 mg/mL to about 20 mg/mL, about 0.05 mg/mL to about 25 mg/mL, about 0.05 mg/mL to about 50 mg/mL, about 0.1 mg/mL to about 0.5 mg/mL, about 0.1 mg/mL to about 1 mg/mL, about 0.1 mg/mL to about 3 mg/mL, about 0.1 mg/mL to about 5 mg/mL, about 0.1 mg/mL to about 10 mg/mL, about 0.1 mg/mL to about 15 mg/mL, about 0.1 mg/mL to about 20 mg/mL, about 0.1 mg/mL to about 25 mg/mL, about 0.1 mg/mL to about 50 mg/mL, about 0.5 mg/mL to about 1 mg/mL, about 0.5 mg/mL to about 3 mg/mL, about 0.5 mg/mL to about 5 mg/mL, about 0.5 mg/mL to about 10 mg/mL, about 0.5 mg/mL to about 15 mg/mL, about 0.5 mg/mL to about 20 mg/mL, about 0.5 mg/mL to about 25 mg/mL, about 0.5 mg/mL to about 50 mg/mL, about 1 mg/mL to about 3 mg/mL, about 1 mg/mL to about 5 mg/mL, about 1 mg/mL to about 10 mg/mL, about 1 mg/mL to about 15 mg/mL, about 1 mg/mL to about 20 mg/mL, about 1 mg/mL to about 25 mg/mL, about 1 mg/mL to about 50 mg/mL, about 3 mg/mL to about 5 mg/mL, about 3 mg/mL to about 10 mg/mL, about 3 mg/mL to about 15 mg/mL, about 3 mg/mL to about 20 mg/mL, about 3 mg/mL to about 25 mg/mL, about 3 mg/mL to about 50 mg/mL, about 5 mg/mL to about 10 mg/mL, about 5 mg/mL to about 15 mg/mL, about 5 mg/mL to about 20 mg/mL, about 5 mg/mL to about 25 mg/mL, about 5 mg/mL to about 50 mg/mL, about 10 mg/mL to about 15 mg/mL, about 10 mg/mL to about 20 mg/mL, about 10 mg/mL to about 25 mg/mL, about 10 mg/mL to about 50 mg/mL, about 15 mg/mL to about 20 mg/mL, about 15 mg/mL to about 25 mg/mL, about 15 mg/mL to about 50 mg/mL, about 20 mg/mL to about 25 mg/mL, about 20 mg/mL to about 50 mg/mL, or about 25 mg/mL to about 50 mg/mL. 1005211In some embodiments, a stock solution of the pharmaceutical composition may comprise L-histidine/L-histidine monohydrochloride buffer, sucrose, and polysorbate. In some cases, a stock solution of the pharmaceutical composition may comprise about 1 mM to about 200 mM, about 2 mM to about 100 mM, about 10 mM to about 50 mM, about 15 mM to about 25 mM, or about 20 mM L-histidine/L-histidine monohydrochloride buffer. In some cases, a stock solution of the pharmaceutical composition may comprise about 1% (w/v) to about 20% (w/v), about 2% (w/v) to about 15% (w/v), 5% (w/v) to about 12% (w/v), about 6% (w/v) to about 10% (w/v), about 8% (w/v) sucrose. In some cases, a stock solution of the pharmaceutical composition may comprise about 0.001% (w/v) to about 0.1% (w/v), about 0.002% (w/v) to about 0.08% (w/v), 0.005% (w/v) to about 0.06% (w/v), about 0.008% (w/v) to about 0.04% (w/v), about 0.01% (w/v) to about 0.03% (w/v), about 0.02% (w/v) polysorbate-80. In some cases, a stock solution of the pharmaceutical composition may comprise the multifunctional molecule at a concentration of about 0.5 mg/mL to about 200 mg/mL, about 1 mg/mL to about 100 mg/mL, about 2 mg/mL to about 80 mg/mL, about 4 mg/mL to about 50 mg/mL, about 6 mg/mL to about 20 mg/mL, about 8 mg/mL to about 12 mg/mL, or about 10 mg/mL. In some cases, a stock solution of the pharmaceutical composition may comprise the multifunctional molecule at a concentration of about 0.5 mg/mL to about 200 mg/mL, about 1 mg/mL to about 100 mg/mL, about 2 mg/mL to about 80 mg/mL, about 4 mg/mL to about 50 mg/mL, about 6 mg/mL to about 20 mg/mL, about 8 mg/mL to about 12 mg/mL, or about 10 mg/mL, about 1 mM to about 200 mM, about 2 mM to about 100 mM, about 10 mM to about 50 mM, about 15 mM to about 25 mM, or about 20 mM L-histidine/L-histidine monohydrochloride buffer, about 1% (w/v) to about 20% (w/v), about 2% (w/v) to about 15% (w/v), 5% (w/v) to about 12% (w/v), about 6% (w/v) to about 10% (w/v), about 8% (w/v) sucrose, and about 0.001% (w/v) to about 0.1% (w/v), about 0.002% (w/v) to about 0.08% (w/v). 0.005% (w/v) to about 0.06% (w/v), about 0.008% (w/v) to about 0.04% (w/v), about 0.01% (w/v) to about 0.03% (w/v), about 0.02% (w/v) polysorbate-80.

### Treatment of Subjects

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In some embodiments, the individual is a human. In some embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In some embodiments, a fetus is treated in utero, e.g., by administering the multifunctional or multispecific molecules or the compositions as described herein to the fetus directly or indirectly (e.g., via the mother).

Suitable routes for administration of the multifunctional or multispecific molecules or the compositions as described herein may vary depending on cell type to which delivery of the multifunctional or multispecific molecules or the compositions is desired. The multifunctional or multispecific molecules or the compositions as described herein may be administered to patients parenterally, for example, by intrathecal injection, intracerebroventricular injection, intraperitoneal injection, intramuscular injection, subcutaneous injection, or intravenous injection.

In some embodiments, the multifunctional or multispecific molecules or the compositions as described herein are administered with one or more agents capable of promoting penetration of the subject the multifunctional or multispecific molecules or the compositions as described herein across the blood-brain barrier by any method known in the art. For example, delivery of agents by administration of an adenovirus vector to motor neurons in muscle tissue is described in U.S. Pat. No. 6,632,427, “Adenoviral-vector-mediated gene transfer into medullary motor neurons,” incorporated herein by reference. Delivery of vectors directly to the brain, e.g., the striatum, the thalamus, the hippocampus, or the substantia nigra, is described, e.g., in U.S. Pat. No. 6,756,523, “Adenovirus vectors for the transfer of foreign genes into cells of the central nervous system particularly in brain,” incorporated herein by reference.

In some embodiments, the multifunctional or multispecific molecules or the compositions as described herein are linked or conjugated with agents that provide desirable pharmaceutical or pharmacodynamic properties. In some embodiments, the multifunctional or multispecific molecules or the compositions as described herein are coupled to a substance, known in the art to promote penetration or transport across the blood-brain barrier, e.g., an antibody to the transferrin receptor. In some embodiments, the multifunctional or multispecific molecules or the compositions as described herein are linked with a viral vector.

In some embodiments, subjects treated using the methods and compositions are evaluated for improvement in condition using any methods known and described in the art.

The terms “treat,” “treating”, and “treatment,” and the like are used herein to generally mean obtaining a desired pharmacological and/or physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly, a human, and includes: (a) preventing the disease from occurring in a subject which may be predisposed to the disease but has not yet been diagnosed as having it; (b) inhibiting the disease. i.e., arresting its development; or (c) relieving the disease. i.e., mitigating or ameliorating the disease and/or its symptoms or conditions. The term “prophylaxis” is used herein to refer to a measure or measures taken for the prevention or partial prevention of a disease or condition. In some embodiments, the terms “condition,” “disease,” or “disorder,” as used herein, are interchangeable.

By “treating or preventing a disease or a disorder” is meant ameliorating any of the conditions or signs or symptoms associated with the disorder before or after it has occurred. As compared with an equivalent untreated control, such reduction or degree of prevention is at least 3%, 5%, 10%, 20%, 40%, 50%, 60%, 80%, 90%, 95%, or 100% as measured by any standard technique. A patient who is being treated for a disease or a disorder, is one who a medical practitioner has diagnosed as having such a condition. Diagnosis may be by any suitable means. Diagnosis and monitoring may involve, for example, detecting the presence of pathological cells in a biological sample (e.g., tissue biopsy, blood test, or urine test), detecting the level of a surrogate marker of the disorder in a biological sample, or detecting symptoms associated with the disorder. A patient in whom the development of a disorder is being prevented may or may not have received such a diagnosis. One in the art will understand that these patients may have been subjected to the same standard tests as described above or may have been identified, without examination, as one at high risk due to the presence of one or more risk factors (e.g., family history or genetic predisposition).

### Methods of Cancer Treatment

Described herein, in certain embodiments, is a method of treating a condition or disease in a subject in need therefor comprising administering to the subject a therapeutically effective amount of the multifunctional polypeptide molecule as described herein, the nucleic acid molecules as described herein, the vector as described herein, the cell as described herein, the pharmaceutical composition as described herein, or a combination thereof, wherein the administering is effective to treat the condition or disease in the subject.

In some embodiments, the condition or disease is cancer. In some embodiments, the cancer is a solid tumor, a hematological cancer, a metastatic cancer, a soft tissue tumor, or a combination thereof. In some embodiments, the cancer is the solid tumor, and wherein the solid tumor is selected from the group consisting of melanoma, pancreatic cancer, breast cancer, colorectal cancer, lung cancer, skin cancer, ovarian cancer, liver cancer, and a combination thereof. In some embodiments, the cancer is the hematological cancer, and wherein the hematological cancer is selected from the group consisting of Hodgkin's lymphoma, Non-Hodgkin's lymphoma, acute myeloid leukemia (AML), chronic myeloid leukemia, myelodysplastic syndrome, multiple myeloma, T-cell lymphoma, acute lymphocytic leukemia, and a combination thereof. In some embodiments, the Non-Hodgkin's lymphoma is selected from the group consisting of B cell lymphoma, diffuse large B cell lymphoma (DLBCL), follicular lymphoma, chronic lymphocytic leukemia (B-CLL), mantle cell lymphoma, marginal zone B-cell lymphoma. Burkitt lymphoma, lymphoplasmacytic lymphoma, hairy cell leukemia, and a combination thereof. In some embodiments, the T-cell lymphoma is peripheral T-cell lymphoma.

In some embodiments, the cancer is characterized by a cancer antigen present on the cancer. In some embodiments, the cancer antigen is a tumor antigen, a stromal antigen, or a hematological antigen. In some embodiments, the cancer antigen is selected from the group consisting of BCMA, CD19, CD20, CD22, FcRH5, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17. Tyrosinase, TRP-1/-2, MC1R, β-catenin, BRCA 1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, α-actinin-4, TRP1/gp75, TRP2, gp100, Melan-A/MART1, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, Li-CAM, CAIX, gpA33, GD3, GM2, VEGFR, Intergrins, carbohydrates, IGF1R, EPHA3, TRAILR1, TRAILR2, RANKL, FAP, TGF-beta, hyaluronic acid, collagen, tenascin C, and tenascin W.

Methods described herein include treating a cancer in a subject by using multispecific or multifunctional molecules as described herein, e.g., using a pharmaceutical composition described herein. Also provided are methods for reducing or ameliorating a symptom of a cancer in a subject, as well as methods for inhibiting the growth of a cancer and/or killing one or more cancer cells. In some embodiments, the methods described herein decrease the size of a tumor and/or decrease the number of cancer cells in a subject administered with a described herein or a pharmaceutical composition described herein.

Described herein are methods of treating a subject having a cancer comprising acquiring a status of one or more TCRβV molecules in a subject. In some embodiments, a higher, e.g., increased, level or activity of one or more TCRβV molecules in a subject, e.g., in a sample from a subject, is indicative of a bias, e.g., a preferential expansion, e.g., clonal expansion, of T cells expressing said one or more TCRβV molecules in the subject.

Without wishing to be bound by theory, it is believed that a biased T cell population, e.g., a T cell population expressing a TCRβV molecule, is antigen-specific for a disease antigen, e.g., a cancer antigen (Wang C Y, et al., Int J Oncol. (2016) 48(6):2247-56). In some embodiments, the cancer antigen comprises a cancer associated antigen or a neoantigen. In some embodiments, a subject having a cancer, e.g., as described herein, has a higher, e.g., increased, level or activity of one or more TCRβV molecules associated with the cancer. In some embodiments, the TCRβV molecule is associated with, e.g., recognizes, a cancer antigen, e.g., a cancer associated antigen or a neoantigen.

Accordingly, as described herein are methods of expanding an immune effector cell population obtained from a subject, comprising acquiring a status of one or more TCRβV molecules in a sample from the subject, comprising contacting said immune effector cell population with an anti-TCRβV antibody molecule as described herein, e.g., an anti-TCRβV antibody molecule that binds to the same TCRβV molecule that is higher, e.g., increased in the immune effector cell population in the sample from the subject. In some embodiments, contacting the population of immune effector cells (e.g., comprising T cells that express one or more TCRβV molecules) with an anti-TCRβV molecule results in expansion of the population of immune effector cells expressing one or more TCRβV molecules. In some embodiments, the expanded population, or a portion thereof, is administered to the subject (e.g., same subject from whom the immune effector cell population was obtained), to treat the cancer. In some embodiments, the expanded population, or a portion thereof, is administered to a different subject (e.g., not the same subject from whom the immune effector cell population was obtained), to treat the cancer.

Also described herein are methods of treating a subject having a cancer, comprising: acquiring a status of one or more TCRβV molecules in a sample from the subject, and determining whether the one or more TCRβV molecules is higher, e.g., increased, in a sample from the subject compared to a reference value, wherein responsive to said determination, administering to the subject an effective amount of an anti-TCRβV antibody molecule, e.g., an agonistic anti-TCRβV antibody molecule, e.g., as described herein. 1005381In some embodiments, the subject has B-CLL. In some embodiments, a subject having B-CLL has a higher, e.g., increased, level or activity of one or more TCRβV molecules, e.g., one or more TCRβV molecules comprising TCRβ V6 subfamily comprising, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ 3 V6-3*01 or TCRβ V6-1*01.

In some embodiments, a subject having B-CLL has a higher, e.g., increased, level or activity of a TCRβ V6 subfamily comprising, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V6 subfamily. In some embodiments, administration of the multifunctional polypeptide molecule as described herein results in expansion of immune cells expressing one or more members of the TCRβ V6 subfamily.

In some embodiments, the subject has melanoma. In some embodiments, a subject having melanoma has a higher, e.g., increased, level or activity of one or more TCRβV molecules, e.g., one or more TCRβV molecules comprising the TCRβ V6 subfamily comprising, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V6 subfamily. In some embodiments, administration of the multifunctional polypeptide molecule as described herein results in expansion of immune cells expressing one or more members of the TCRβ V6 subfamily.

In some embodiments, acquiring a value for the status, e.g., presence, level and/or activity, of one or more TCRβV molecules comprises acquiring a measure of the T cell receptor (TCR) repertoire of a sample. In some embodiments, the value comprises a measure of the clonotype of a population of T cells in the sample.

In some embodiments, a value for the status of one or more TCRβV molecules is obtained, e.g., measured, using an assay described in Wang C Y, et al., Int J Oncol. (2016) 48(6):2247-56, the entire contents of which are hereby incorporated by reference.

In some embodiments, a value for the status of one or more TCRβV molecules is obtained, e.g., measured, using flow cytometry.

### Combination Therapies

In some embodiments, the method as described herein further comprises administering a second therapeutic agent or therapy to the subject.

In some embodiments, the second therapeutic agent or therapy comprises a chemotherapeutic agent, a biologic agent, a hormonal therapy, radiation, or surgery.

In some embodiments, the second therapeutic agent or therapy is administered in combination with the multifunctional polypeptide molecule as described herein, the nucleic acid molecules as described herein, the vector as described herein, the cell as described herein, the pharmaceutical composition as described herein, sequentially, simultaneously, or concurrently.

The multispecific or multifunctional molecules as described herein can be used in combination with a second therapeutic agent or procedure.

In some embodiments, the multispecific or multifunctional molecules as described herein and the second therapeutic agent or procedure are administered/performed after a subject has been diagnosed with a cancer, e.g., before the cancer has been eliminated from the subject. In some embodiments, the multispecific or multifunctional molecules as described herein and the second therapeutic agent or procedure are administered/performed simultaneously or concurrently. For example, the delivery of one treatment is still occurring when the delivery of the second commences, e.g., there is an overlap in administration of the treatments. In other embodiments, the multispecific or multifunctional molecules as described herein and the second therapeutic agent or procedure are administered/performed sequentially. For example, the delivery of one treatment ceases before the delivery of the other treatment begins.

In some embodiments, combination therapy can lead to more effective treatment than monotherapy with either agent alone. In some embodiments, the combination of the first and second treatment is more effective (e.g., leads to a greater reduction in symptoms and/or cancer cells) than the first or second treatment alone. In some embodiments, the combination therapy permits use of a lower dose of the first or the second treatment compared to the dose of the first or second treatment normally required to achieve similar effects when administered as a monotherapy. In some embodiments, the combination therapy has a partially additive effect, wholly additive effect, or greater than additive effect.

In some embodiments, the anti-TCRβV antibody, multispecific or multifunctional molecule is administered in combination with a therapy, e.g., a cancer therapy (e.g., one or more of anti-cancer agents, immunotherapy, photodynamic therapy (PDT), surgery and/or radiation). The terms “chemotherapeutic.” “chemotherapeutic agent.” and “anti-cancer agent” are used interchangeably herein. The administration of the multispecific or multifunctional molecule and the therapy, e.g., the cancer therapy, can be sequential (with or without overlap) or simultaneous. Administration of the anti-TCRβV antibody, multispecific or multifunctional molecule can be continuous or intermittent during the course of therapy (e.g., cancer therapy). Certain therapies described herein can be used to treat cancers and non-cancerous diseases. For example, PDT efficacy can be enhanced in cancerous and non-cancerous conditions (e.g., tuberculosis) using the methods and compositions described herein (reviewed in, e.g., Agostinis, P. et al. (2011) CA Cancer J. Clin. 61:250-281).

Methods described herein include treating a cancer in a subject by using the multispecific or multifunctional molecules as described herein, e.g., using a pharmaceutical composition as described herein. Also provided are methods for reducing or ameliorating a symptom of a cancer in a subject, as well as methods for inhibiting the growth of a cancer and/or killing one or more cancer cells. In some embodiments, the methods described herein decrease the size of a tumor and/or decrease the number of cancer cells in a subject administered with a described herein or a pharmaceutical composition described herein.

In some embodiments, the cancer is a hematological cancer. In some embodiments, the hematological cancer is a leukemia or a lymphoma. As used herein, a “hematologic cancer” refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sezary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

In some embodiments, the cancer is a myeloproliferative neoplasm, e.g., primary or idiopathic myelofibrosis (MF), essential thrombocytosis (ET), polycythemia vera (PV), or chronic myelogenous leukemia (CML). In some embodiments, the cancer is myelofibrosis. In some embodiments, the subject has myelofibrosis. In some embodiments, the subject has a calreticulin mutation, e.g., a calreticulin mutation as described herein. In some embodiments, the subject does not have the JAK2-V617F mutation. In some embodiments, the subject has the JAK2-V617F mutation. In some embodiments, the subject has a MPL mutation. In some embodiments, the subject does not have a MPL mutation.

In some embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi's sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

In some embodiments, the cancer is acute lymphoblastic leukemia, acute lymphocytic leukemia, acute myelogenous leukemia, aplastic anemia, chronic myelogenous leukemia, desmoplastic small round cell tumor, Ewing's sarcoma, Hodgkin's disease, multiple myeloma, myelodysplasia, Non-Hodgkin's lymphoma, paroxysmal nocturnal hemoglobinuria, radiation poisoning, chronic lymphocytic leukemia, AL amyloidosis, essential thrombocytosis, polycythemia vera, severe aplastic anemia, neuroblastoma, breast tumors, ovarian tumors, renal cell carcinoma, autoimmune disorders, such as systemic sclerosis, osteopetrosis, inherited metabolic disorders, juvenile chronic arthritis, adrenoleukodystrophy, amegakaryocytic thrombocytopenia, sickle cell disease, severe congenital immunodeficiency, Griscelli syndrome type II, Hurler syndrome, Kostmann syndrome, Krabbe disease, metachromatic leukodystrophy, thalassemia, hemophagocytic lymphohistiocytosis, and Wiskott-Aldrich syndrome, leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive. ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g., non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myceloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease. Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer. In some embodiments, the cancer is a solid tumor. In some embodiments, the cancer is hematological.

In some embodiments, the multispecific or multifunctional molecules as described herein (or pharmaceutical composition as described herein) are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition (or multispecific or multifunctional molecules) to be administered can be determined by a physician with consideration of individual differences in tumor size, extent of infection or metastasis, age, weight, and condition of the subject. In some embodiments, the pharmaceutical composition described herein can be administered at a dosage of 10⁴ to 10⁹ cells/kg body weight, e.g., 10⁵ to 10⁶ cells/kg body weight, including all integer values within those ranges. In some embodiments, the pharmaceutical composition described herein can be administered multiple times at these dosages. In some embodiments, the pharmaceutical composition described herein can be administered using infusion techniques described in immunotherapy (see, e.g., Rosenberg et al., New Eng. J. of Med. 319:1676, 1988).

In some embodiments, the multispecific or multifunctional molecules as described herein or the pharmaceutical composition as described herein is administered to the subject parentally. In some embodiments, the cells are administered to the subject intravenously, subcutaneously, intratumorally, intranodally, intramuscularly, intradermally, or intraperitoneally. In some embodiments, the cells are administered, e.g., injected, directly into a tumor or lymph node. In some embodiments, the cells are administered as an infusion (e.g., as described in Rosenberg et al., New Eng. J. of Med. 319:1676, 1988) or an intravenous push. In some embodiments, the cells are administered as an injectable depot formulation.

In some embodiments, the multispecific or multifunctional molecules as described herein may be administered at a dose of at least about 0.0001 mg/kg, at least about 0.0005 mg/kg, 0.001 mg/kg, at least about 0.005 mg/kg, at least about 0.01 mg/kg, at least about 0.05 mg/kg, at least about 0.1 mg/kg, at least about 0.5 mg/kg, at least about 1 mg/kg, at least about 2 mg/kg, at least about 3 mg/kg, at least about 4 mg/kg, at least about 5 mg/kg, at least about 6 mg/kg, at least about 7 mg/kg, at least about 8 mg/kg, at least about 9 mg/kg, at least about 10 mg/kg, at least about 11 mg/kg, at least about 12 mg/kg, at least about 13 mg/kg, at least about 14 mg/kg, at least about 15 mg/kg, at least about 20 mg/kg, at least about 30 mg/kg, at least about 40 mg/kg, at least about 50 mg/kg, at least about 60 mg/kg, or at least about 70 mg/kg. In some embodiments, the multispecific or multifunctional molecules as described herein may be administered at a dose of at most about 70 mg/kg, at most about 60 mg/kg, at most about 50 mg/kg, at most about 40 mg/kg, at most about 30 mg/kg, at most about 20 mg/kg, at most about 15 mg/kg, at most about 14 mg/kg, at most about 13 mg/kg, at most about 12 mg/kg, at most about 11 mg/kg, at most about 10 mg/kg, at most about 9 mg/kg, at most about 8 mg/kg, at most about 7 mg/kg, at most about 6 mg/kg, at most about 5 mg/kg, at most about 4 mg/kg, at most about 3 mg/kg, at most about 2 mg/kg, at most about 1 mg/kg, at most about 0.5 mg/kg, at most about 0.1 mg/kg, at most about 0.05 mg/kg, at most about 0.01 mg/kg, at most about 0.005 mg/kg, at most about 0.001 mg/kg, at most about 0.0005 mg/kg, or at most about 0.0001 mg/kg.

In some embodiments, the multispecific or multifunctional molecules as described herein may be administered at a dose from about 0.0001 mg/kg to about 5 mg/kg. In some embodiments, the multispecific or multifunctional molecules as described herein may be administered at a dose from about 0.0001 mg/kg to about 0.001 mg/kg, about 0.0001 mg/kg to about 0.005 mg/kg, about 0.0001 mg/kg to about 0.01 mg/kg, about 0.0001 mg/kg to about 0.05 mg/kg, about 0.0001 mg/kg to about 0.1 mg/kg, about 0.0001 mg/kg to about 0.5 mg/kg, about 0.0001 mg/kg to about 1 mg/kg, about 0.0001 mg/kg to about 2 mg/kg, about 0.0001 mg/kg to about 3 mg/kg, about 0.0001 mg/kg to about 4 mg/kg, about 0.0001 mg/kg to about 5 mg/kg, about 0.001 mg/kg to about 0.005 mg/kg, about 0.001 mg/kg to about 0.01 mg/kg, about 0.001 mg/kg to about 0.05 mg/kg, about 0.001 mg/kg to about 0.1 mg/kg, about 0.001 mg/kg to about 0.5 mg/kg, about 0.001 mg/kg to about 1 mg/kg, about 0.001 mg/kg to about 2 mg/kg, about 0.001 mg/kg to about 3 mg/kg, about 0.001 mg/kg to about 4 mg/kg, about 0.001 mg/kg to about 5 mg/kg, about 0.005 mg/kg to about 0.01 mg/kg, about 0.005 mg/kg to about 0.05 mg/kg, about 0.005 mg/kg to about 0.1 mg/kg, about 0.005 mg/kg to about 0.5 mg/kg, about 0.005 mg/kg to about 1 mg/kg, about 0.005 mg/kg to about 2 mg/kg, about 0.005 mg/kg to about 3 mg/kg, about 0.005 mg/kg to about 4 mg/kg, about 0.005 mg/kg to about 5 mg/kg, about 0.01 mg/kg to about 0.05 mg/kg, about 0.01 mg/kg to about 0.1 mg/kg, about 0.01 mg/kg to about 0.5 mg/kg, about 0.01 mg/kg to about 1 mg/kg, about 0.01 mg/kg to about 2 mg/kg, about 0.01 mg/kg to about 3 mg/kg, about 0.01 mg/kg to about 4 mg/kg, about 0.01 mg/kg to about 5 mg/kg, about 0.05 mg/kg to about 0.1 mg/kg, about 0.05 mg/kg to about 0.5 mg/kg, about 0.05 mg/kg to about 1 mg/kg, about 0.05 mg/kg to about 2 mg/kg, about 0.05 mg/kg to about 3 mg/kg, about 0.05 mg/kg to about 4 mg/kg, about 0.05 mg/kg to about 5 mg/kg, about 0.1 mg/kg to about 0.5 mg/kg, about 0.1 mg/kg to about 1 mg/kg, about 0.1 mg/kg to about 2 mg/kg, about 0.1 mg/kg to about 3 mg/kg, about 0.1 mg/kg to about 4 mg/kg, about 0.1 mg/kg to about 5 mg/kg, about 0.5 mg/kg to about 1 mg/kg, about 0.5 mg/kg to about 2 mg/kg, about 0.5 mg/kg to about 3 mg/kg, about 0.5 mg/kg to about 4 mg/kg, about 0.5 mg/kg to about 5 mg/kg, about 1 mg/kg to about 2 mg/kg, about 1 mg/kg to about 3 mg/kg, about 1 mg/kg to about 4 mg/kg, about 1 mg/kg to about 5 mg/kg, about 2 mg/kg to about 3 mg/kg, about 2 mg/kg to about 4 mg/kg, about 2 mg/kg to about 5 mg/kg, about 3 mg/kg to about 4 mg/kg, about 3 mg/kg to about 5 mg/kg, or about 4 mg/kg to about 5 mg/kg.

In some embodiments, the multispecific or multifunctional molecules as described herein may be administered at a dose from about 5 mg/kg to about 50 mg/kg. In some embodiments, the multispecific or multifunctional molecules as described herein may be administered at a dose from about 5 mg/kg to about 6 mg/kg, about 5 mg/kg to about 7 mg/kg, about 5 mg/kg to about 8 mg/kg, about 5 mg/kg to about 9 mg/kg, about 5 mg/kg to about 10 mg/kg, about 5 mg/kg to about 12 mg/kg, about 5 mg/kg to about 15 mg/kg, about 5 mg/kg to about 20 mg/kg, about 5 mg/kg to about 30 mg/kg, about 5 mg/kg to about 40 mg/kg, about 5 mg/kg to about 50 mg/kg, about 6 mg/kg to about 7 mg/kg, about 6 mg/kg to about 8 mg/kg, about 6 mg/kg to about 9 mg/kg, about 6 mg/kg to about 10 mg/kg, about 6 mg/kg to about 12 mg/kg, about 6 mg/kg to about 15 mg/kg, about 6 mg/kg to about 20 mg/kg, about 6 mg/kg to about 30 mg/kg, about 6 mg/kg to about 40 mg/kg, about 6 mg/kg to about 50 mg/kg, about 7 mg/kg to about 8 mg/kg, about 7 mg/kg to about 9 mg/kg, about 7 mg/kg to about 10 mg/kg, about 7 mg/kg to about 12 mg/kg, about 7 mg/kg to about 15 mg/kg, about 7 mg/kg to about 20 mg/kg, about 7 mg/kg to about 30 mg/kg, about 7 mg/kg to about 40 mg/kg, about 7 mg/kg to about 50 mg/kg, about 8 mg/kg to about 9 mg/kg, about 8 mg/kg to about 10 mg/kg, about 8 mg/kg to about 12 mg/kg, about 8 mg/kg to about 15 mg/kg, about 8 mg/kg to about 20 mg/kg, about 8 mg/kg to about 30 mg/kg, about 8 mg/kg to about 40 mg/kg, about 8 mg/kg to about 50 mg/kg, about 9 mg/kg to about 10 mg/kg, about 9 mg/kg to about 12 mg/kg, about 9 mg/kg to about 15 mg/kg, about 9 mg/kg to about 20 mg/kg, about 9 mg/kg to about 30 mg/kg, about 9 mg/kg to about 40 mg/kg, about 9 mg/kg to about 50 mg/kg, about 10 mg/kg to about 12 mg/kg, about 10 mg/kg to about 15 mg/kg, about 10 mg/kg to about 20 mg/kg, about 10 mg/kg to about 30 mg/kg, about 10 mg/kg to about 40 mg/kg, about 10 mg/kg to about 50 mg/kg, about 12 mg/kg to about 15 mg/kg, about 12 mg/kg to about 20 mg/kg, about 12 mg/kg to about 30 mg/kg, about 12 mg/kg to about 40 mg/kg, about 12 mg/kg to about 50 mg/kg, about 15 mg/kg to about 20 mg/kg, about 15 mg/kg to about 30 mg/kg, about 15 mg/kg to about 40 mg/kg, about 15 mg/kg to about 50 mg/kg, about 20 mg/kg to about 30 mg/kg, about 20 mg/kg to about 40 mg/kg, about 20 mg/kg to about 50 mg/kg, about 30 mg/kg to about 40 mg/kg, about 30 mg/kg to about 50 mg/kg, or about 40 mg/kg to about 50 mg/kg.

In some embodiments, the multispecific or multifunctional molecules as described herein may be administered at a dose of at least about 0.006 mg, at least about 0.03 mg, 0.06 mg, at least about 0.3 mg, at least about 0.6 mg, at least about 3 mg, at least about 6 mg, at least about 30 mg, at least about 60 mg, at least about 120 mg, at least about 180 mg, at least about 240 mg, at least about 300 mg, at least about 360 mg, at least about 420 mg, at least about 480 mg, at least about 540 mg, at least about 600 mg, at least about 660 mg, at least about 720 mg, at least about 780 mg, at least about 840 mg, at least about 900 mg, at least about 1200 mg, at least about 1800 mg, at least about 2400 mg, at least about 3000 mg, at least about 3600 mg, or at least about 4200 mg. In some embodiments, the multispecific or multifunctional molecules as described herein may be administered at a dose of at most about 4200 mg, at most about 3600 mg, at most about 3000 mg, at most about 2400 mg, at most about 1800 mg, at most about 1200 mg, at most about 900 mg, at most about 840 mg, at most about 780 mg, at most about 720 mg, at most about 660 mg, at most about 600 mg, at most about 540 mg, at most about 480 mg, at most about 420 mg, at most about 360 mg, at most about 300 mg, at most about 240 mg, at most about 180 mg, at most about 120 mg, at most about 60 mg, at most about 30 mg, at most about 6 mg, at most about 3 mg, at most about 0.6 mg, at most about 0.3 mg, at most about 0.06 mg, at most about 0.03 mg, or at most about 0.006 mg.

In some embodiments, the multispecific or multifunctional molecules as described herein may be administered at a dose from about 0.006 mg to about 300 mg. In some embodiments, the multispecific or multifunctional molecules as described herein may be administered at a dose from about 0.006 mg to about 0.06 mg, about 0.006 mg to about 0.3 mg, about 0.006 mg to about 0.6 mg, about 0.006 mg to about 3 mg, about 0.006 mg to about 6 mg, about 0.006 mg to about 30 mg, about 0.006 mg to about 60 mg, about 0.006 mg to about 120 mg, about 0.006 mg to about 180 mg, about 0.006 mg to about 240 mg, about 0.006 mg to about 300 mg, about 0.06 mg to about 0.3 mg, about 0.06 mg to about 0.6 mg, about 0.06 mg to about 3 mg, about 0.06 mg to about 6 mg, about 0.06 mg to about 30 mg, about 0.06 mg to about 60 mg, about 0.06 mg to about 120 mg, about 0.06 mg to about 180 mg, about 0.06 mg to about 240 mg, about 0.06 mg to about 300 mg, about 0.3 mg to about 0.6 mg, about 0.3 mg to about 3 mg, about 0.3 mg to about 6 mg, about 0.3 mg to about 30 mg, about 0.3 mg to about 60 mg, about 0.3 mg to about 120 mg, about 0.3 mg to about 180 mg, about 0.3 mg to about 240 mg, about 0.3 mg to about 300 mg, about 0.6 mg to about 3 mg, about 0.6 mg to about 6 mg, about 0.6 mg to about 30 mg, about 0.6 mg to about 60 mg, about 0.6 mg to about 120 mg, about 0.6 mg to about 180 mg, about 0.6 mg to about 240 mg, about 0.6 mg to about 300 mg, about 3 mg to about 6 mg, about 3 mg to about 30 mg, about 3 mg to about 60 mg, about 3 mg to about 120 mg, about 3 mg to about 180 mg, about 3 mg to about 240 mg, about 3 mg to about 300 mg, about 6 mg to about 30 mg, about 6 mg to about 60 mg, about 6 mg to about 120 mg, about 6 mg to about 180 mg, about 6 mg to about 240 mg, about 6 mg to about 300 mg, about 30 mg to about 60 mg, about 30 mg to about 120 mg, about 30 mg to about 180 mg, about 30 mg to about 240 mg, about 30 mg to about 300 mg, about 60 mg to about 120 mg, about 60 mg to about 180 mg, about 60 mg to about 240 mg, about 60 mg to about 300 mg, about 120 mg to about 180 mg, about 120 mg to about 240 mg, about 120 mg to about 300 mg, about 180 mg to about 240 mg, about 180 mg to about 300 mg, or about 240 mg to about 300 mg.

In some embodiments, the multispecific or multifunctional molecules as described herein may be administered at a dose from about 300 mg to about 3,000 mg. In some embodiments, the multispecific or multifunctional molecules as described herein may be administered at a dose from about 300 mg to about 360 mg, about 300 mg to about 420 mg, about 300 mg to about 480 mg, about 300 mg to about 540 mg, about 300 mg to about 600 mg, about 300 mg to about 720 mg, about 300 mg to about 900 mg, about 300 mg to about 1,200 mg, about 300 mg to about 1,800 mg, about 300 mg to about 2,400 mg, about 300 mg to about 3,000 mg, about 360 mg to about 420 mg, about 360 mg to about 480 mg, about 360 mg to about 540 mg, about 360 mg to about 600 mg, about 360 mg to about 720 mg, about 360 mg to about 900 mg, about 360 mg to about 1,200 mg, about 360 mg to about 1,800 mg, about 360 mg to about 2,400 mg, about 360 mg to about 3,000 mg, about 420 mg to about 480 mg, about 420 mg to about 540 mg, about 420 mg to about 600 mg, about 420 mg to about 720 mg, about 420 mg to about 900 mg, about 420 mg to about 1,200 mg, about 420 mg to about 1,800 mg, about 420 mg to about 2,400 mg, about 420 mg to about 3,000 mg, about 480 mg to about 540 mg, about 480 mg to about 600 mg, about 480 mg to about 720 mg, about 480 mg to about 900 mg, about 480 mg to about 1,200 mg, about 480 mg to about 1,800 mg, about 480 mg to about 2,400 mg, about 480 mg to about 3,000 mg, about 540 mg to about 600 mg, about 540 mg to about 720 mg, about 540 mg to about 900 mg, about 540 mg to about 1,200 mg, about 540 mg to about 1,800 mg, about 540 mg to about 2,400 mg, about 540 mg to about 3,000 mg, about 600 mg to about 720 mg, about 600 mg to about 900 mg, about 600 mg to about 1,200 mg, about 600 mg to about 1,800 mg, about 600 mg to about 2,400 mg, about 600 mg to about 3,000 mg, about 720 mg to about 900 mg, about 720 mg to about 1,200 mg, about 720 mg to about 1,800 mg, about 720 mg to about 2,400 mg, about 720 mg to about 3,000 mg, about 900 mg to about 1,200 mg, about 900 mg to about 1,800 mg, about 900 mg to about 2,400 mg, about 900 mg to about 3,000 mg, about 1,200 mg to about 1,800 mg, about 1,200 mg to about 2,400 mg, about 1,200 mg to about 3,000 mg, about 1,800 mg to about 2,400 mg, about 1,800 mg to about 3,000 mg, or about 2,400 mg to about 3,000 mg.

In some embodiments, the multispecific or multifunctional molecules as described herein (or pharmaceutical composition as described herein) may be administered to a subject in need thereof over a time course. In some embodiments, the multispecific or multifunctional molecules as described herein may be administered over a time course of at least about 10 minutes, at least about 15 minutes, at least about 20 minutes, at least about 25 minutes, at least about 30 minutes, at least about 45 minutes, at least about 60 minutes, at least about 75 minutes, at least about 90 minutes, at least about 105 minutes, at least about 120 minutes, at least about 150 minutes, at least about 180 minutes, at least about 210 minutes, at least about 240 minutes, at least about 270 minutes, or at least about 300 minutes. In some embodiments, the multispecific or multifunctional molecules as described herein may be administered over a time course of at most about 300 minutes, at most about 270 minutes, at most about 240 minutes, at most about 210 minutes, at most about 180 minutes, at most about 150 minutes, at most about 120 minutes, at most about 105 minutes, at most about 90 minutes, at most about 75 minutes, at most about 60 minutes, at most about 45 minutes, at most about 30 minutes, at most about 25 minutes, at most about 20 minutes, at most about 15 minutes, or at most about 10 minutes. In some embodiments, the multispecific or multifunctional molecules as described herein may be administered over a time course of from about 15 minutes to about 300 minutes. In some embodiments, the multispecific or multifunctional molecules as described herein may be administered over a time course of from about 15 minutes to about 30 minutes, about 15 minutes to about 45 minutes, about 15 minutes to about 60 minutes, about 15 minutes to about 90 minutes, about 15 minutes to about 120 minutes, about 15 minutes to about 150 minutes, about 15 minutes to about 180 minutes, about 15 minutes to about 210 minutes, about 15 minutes to about 240 minutes, about 15 minutes to about 270 minutes, about 15 minutes to about 300 minutes, about 30 minutes to about 45 minutes, about 30 minutes to about 60 minutes, about 30 minutes to about 90 minutes, about 30 minutes to about 120 minutes, about 30 minutes to about 150 minutes, about 30 minutes to about 180 minutes, about 30 minutes to about 210 minutes, about 30 minutes to about 240 minutes, about 30 minutes to about 270 minutes, about 30 minutes to about 300 minutes, about 45 minutes to about 60 minutes, about 45 minutes to about 90 minutes, about 45 minutes to about 120 minutes, about 45 minutes to about 150 minutes, about 45 minutes to about 180 minutes, about 45 minutes to about 210 minutes, about 45 minutes to about 240 minutes, about 45 minutes to about 270 minutes, about 45 minutes to about 300 minutes, about 60 minutes to about 90 minutes, about 60 minutes to about 120 minutes, about 60 minutes to about 150 minutes, about 60 minutes to about 180 minutes, about 60 minutes to about 210 minutes, about 60 minutes to about 240 minutes, about 60 minutes to about 270 minutes, about 60 minutes to about 300 minutes, about 90 minutes to about 120 minutes, about 90 minutes to about 150 minutes, about 90 minutes to about 180 minutes, about 90 minutes to about 210 minutes, about 90 minutes to about 240 minutes, about 90 minutes to about 270 minutes, about 90 minutes to about 300 minutes, about 120 minutes to about 150 minutes, about 120 minutes to about 180 minutes, about 120 minutes to about 210 minutes, about 120 minutes to about 240 minutes, about 120 minutes to about 270 minutes, about 120 minutes to about 300 minutes, about 150 minutes to about 180 minutes, about 150 minutes to about 210 minutes, about 150 minutes to about 240 minutes, about 150 minutes to about 270 minutes, about 150 minutes to about 300 minutes, about 180 minutes to about 210 minutes, about 180 minutes to about 240 minutes, about 180 minutes to about 270 minutes, about 180 minutes to about 300 minutes, about 210 minutes to about 240 minutes, about 210 minutes to about 270 minutes, about 210 minutes to about 300 minutes, about 240 minutes to about 270 minutes, about 240 minutes to about 300 minutes, or about 270 minutes to about 300 minutes.

In some embodiments, the multispecific or multifunctional molecules as described herein may be administered as multiple doses to a subject in need thereof. In some embodiments, the multispecific or multifunctional molecules as described herein may be administered in 1 dose, 2 doses, 3 doses, 4 doses, 5 doses, 6 doses, 7 doses, 8 doses, 9 doses, 10 doses, or more. In some embodiments, a subject may be administered an initial dose of the multispecific or multifunctional molecules as described herein and a subsequent dose that is lower than an initial dose. In some embodiments, a subject may be administered an initial dose of the multispecific or multifunctional molecules as described herein and a subsequent dose that is higher than an initial dose. In some embodiments, a subject may be administered an initial dose of the multispecific or multifunctional molecules as described herein and a subsequent dose that is the same as an initial dose.

In some embodiments, an initial dose of the multispecific or multifunctional molecules as described herein may not be well tolerated and a subject may be administered a subsequent dose that is lower than the initial dose. In some embodiments, an initial dose of the multispecific or multifunctional molecules as described herein may be well tolerated and a subject may be administered a subsequent dose that is the same as the initial dose. In some embodiments, an initial dose of the multispecific or multifunctional molecules as described herein may be well tolerated and a subject may be administered a subsequent dose that is higher than the initial dose. In some embodiments, an initial dose of the multispecific or multifunctional molecules as described herein may be effective and a subject may be administered a subsequent dose that is the same as the initial dose. In some embodiments, an initial dose of the multispecific or multifunctional molecules as described herein may be effective and a subject may be administered a subsequent dose that is higher than the initial dose.

In some embodiments, multiple doses of the multispecific or multifunctional molecules as described herein may be spaced out by 1 minute, 2 minutes, 3 minutes, 4 minutes, 5 minutes, 10 minutes, 15 minutes, 30 minutes, 1 hour, 2 hours, 3 hours, 4 hours, 5 hours, 6 hours, 12 hours, 18 hours, 1 day, 2 days, 3 days, 4 days, 5 days, 6 days, 1 week, 2 weeks, 3 weeks, or 4 weeks. A dose of the multispecific or multifunctional molecules as described herein may be followed by a subsequent dose immediately after a previous dose.

In some embodiments, a dose or multiple doses of the multispecific or multifunctional molecules as described herein may be administered over a 7-day cycle, a 10-day cycle, a 14-day cycle, a 17-day cycle, a 21-day cycle, a 24-day cycle, a 28-day cycle, a 32-day cycle, a 35-day cycle, a 38-day cycle, a 42-day cycle, a 45-day cycle, a 49-day cycle, a 54-day cycle, a 56-day cycle, a 60-day cycle, a 63-day cycle, a 65-day cycle, or a 70-day cycle. Within an administration cycle, an intravenous infusion of the multispecific or multifunctional molecules as described herein may be administered every 2 days, every 3 days, every 4 days, every 5 days, every 6 days, every 7 days, every 8 days, every 9 days, every 10 days, every 11 days, every 12 days, every 13 days, every 14 days, every 14 days, every 16 days, every 17 days, every 18 days, every 19 days, every 20 days, every 21 days, or every month.

Multiple doses of the multispecific or multifunctional molecules as described herein may be administered wherein each subsequent dose may be higher than the previous dose. In some embodiments, a first dose may be administered to a subject in need thereof and a second dose may be administered that is higher than the first dose. In some embodiments, a second dose may be administered to a subject in need thereof and a third dose may be administered that is higher than the second dose. In some embodiments, a third dose may be administered to a subject in need thereof and a fourth dose may be administered that is higher than the third dose. In some embodiments, a fourth dose may be administered to a subject in need thereof and a fifth dose may be administered that is higher than the fourth dose. In some embodiments, a fifth dose may be administered to a subject in need thereof and a sixth dose may be administered that is higher than the fifth dose. In some embodiments, the interval between a first dose, a second dose, a third dose, a fourth dose, a fifth dose, and/or a sixth dose may be less than about 1 minutes, less than about 2 minutes, less than about 3 minutes, less than about 4 minutes, less than about 5 minutes, less than about 10 minutes, less than about 15 minutes, less than about 30 minutes, less than about 1 hour, less than about 2 hours, less than about 3 hours, less than about 4 hours, less than about 5 hours, less than about 6 hours, less than about 12 hours, less than about 18 hours, less than about 1 day, less than about 2 days, less than about 3 days, less than about 4 days, less than about 5 days, less than about 6 days, less than about 1 week, less than about 2 weeks, less than about 3 weeks, or less than about 4 weeks. In some embodiments, increasing a dose of the multispecific or multifunctional molecules as described herein from a previous dose amount may alleviate and/or reduce symptoms of cytokine release syndrome (CRS). In some embodiments, increasing a dose of the multispecific or multifunctional molecules as described herein from a previous dose amount may prevent symptoms of cytokine release syndrome (CRS).

In some embodiments, the subject is a mammal. In some embodiments, the subject is a human, monkey, pig, dog, cat, cow, sheep, goat, rabbit, rat, or mouse. In some embodiments, the subject is a human. In some embodiments, the subject is a pediatric subject, e.g., less than 18 years of age. e.g., less than 17, 16, 15, 14, 13, 12, 11, 10, 9, 8, 7, 6, 5, 4, 3, 2, 1 or less years of age. In some embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

### Anti-Cancer Therapies

In other embodiments, the multispecific or multifunctional molecules as described herein is administered in combination with a low or small molecular weight chemotherapeutic agent. Exemplary low or small molecular weight chemotherapeutic agents include, but not limited to, 13-cis-retinoic acid (isotretinoin, ACCUTANE®), 2-CdA (2-chlorodeoxyadenosine, cladribine, LEUSTATIN™), 5-azacitidine (azacitidine, VIDAZA®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomycin, COSMEGEN®), alitretinoin (PANREITN®), all-transretinoic acid (ATRA, tretinoin, VESANOID®), altretamine (hexamethylmelamine, HMM, HEXALEN(®), amethopterin (methotrexate, methotrexate sodium, MTX, TREXALL™, RHEUMATREX®), amifostine (ETHYOL®), arabinosylcytosine (Ara-C, cytarabine, CYTOSAR-UV), arsenic trioxide (TRISENOX®), asparaginase (Erwinia L-asparaginase, L-asparaginase, ELSPAR®, KIDROLASE®), BCNU (carmustine, BiCNU®), bendamustine (TREANDA®), bexarotene (TARGRETIN®), bleomycin (BLENOXANE®), busulfan (BUSULFEX®, MYLERAN®), calcium leucovorin (Citrovorum Factor, folinic acid, leucovorin), camptothecin-11 (CPT-11, irinotecan, CAMPTOSAR®), capecitabine (XELODA®), carboplatin (PARAPLATIN®), carmustine wafer (prolifeprospan 20 with carmustine implant. GLIADEL® wafer), CCI-779 (temsirolimus, TORISEL®), CCNU (lomustine, CeeNU), CDDP (cisplatin, PLATINOL®, PLATINOL-AQ®), chlorambucil (leukeran), cyclophosphamide (CYTOXAN®, NEOSAR®), dacarbazine (DIC, DTIC, imidazole carboxamide, DTIC-DOME®), daunomycin (daunorubicin, daunorubicin hydrochloride, rubidomycin hydrochloride, CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), estramustine (EMCYT®), etoposide (VP-16, etoposide phosphate, TOPOSAR®, VEPESID®, ETOPOPHOS®), floxuridine (FUDR®), fludarabine (FLUDARA®), fluorouracil (cream) (CARAC™, EFUDEX®, FLUOROPLEX®), gemcitabine (GEMZAR®), hydroxyurea (HYDREA®, DROXIA™, MYLOCEL™), idarubicin (IDAMYCIN®), ifosfamide (IFEX®), ixabepilone (IXEMPRA™), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard, MUSTARGEN®), mesna (MESNEX™), mitomycin (mitomycin-C, MTC, MUTAMYCIN®), nelarabine (ARRANON®), oxaliplatin (ELOXATIN™, paclitaxel (TAXOL®, ONXAL™), pegaspargase (PEG-L-asparaginase, ONCOSPAR®), PEMETREXED (ALIMTA®), pentostatin (NIPENTX®), procarbazine (MATULANE®), streptozocin (ZANOSAR®), temozolomide (TEMODAR(®), teniposide (VM-26, VUMON®), TESPA (thiophosphoamide, thiotepa, TSPA, THIOPLEX®), topotecan (HYCAMTIN®), vinblastine (vinblastine sulfate, vincaleukoblastine, VLB, ALKABAN-AQ®, VELBAN®), vinorelbine (vinorelbine tartrate, NAVELBINE®), and vorinostat (ZOLINZA®).

In another embodiment, the multispecific or multifunctional molecules as described herein is administered in conjunction with a biologic. Biologics useful in the treatment of cancers are known in the art and a binding molecule as described herein may be administered, for example, in conjunction with such known biologics. For example, the FDA has approved the following biologics for the treatment of breast cancer: HERCEPTIN® (trastuzumab, Genentech Inc., South San Francisco, Calif.; a humanized monoclonal antibody that has anti-tumor activity in HER2-positive breast cancer); FASLODEX® (fulvestrant, AstraZeneca Pharmaceuticals, LP, Wilmington, Del.; an estrogen-receptor antagonist used to treat breast cancer); ARIMIDEX® (anastrozole, AstraZeneca Pharmaceuticals, LP; a nonsteroidal aromatase inhibitor which blocks aromatase, an enzyme needed to make estrogen); Aromasin, (exemestane, Pfizer Inc., New York, N.Y.; an irreversible, steroidal aromatase inactivator used in the treatment of breast cancer); FEMARA) (letrozole, Novartis Pharmaceuticals, East Hanover, N.J.; a nonsteroidal aromatase inhibitor approved by the FDA to treat breast cancer); and NOLVADEX® (tamoxifen, AstraZeneca Pharmaceuticals. LP; a nonsteroidal antiestrogen approved by the FDA to treat breast cancer). Other biologics with which the binding molecules as described herein may be combined include: AVASTIN® (bevacizumab, Genentech Inc.; the first FDA-approved therapy designed to inhibit angiogenesis); and ZEVALINX® (ibritumomab tiuxetan, Biogen Idec, Cambridge, Mass.; a radiolabeled monoclonal antibody currently approved for the treatment of B-cell lymphomas).

In addition, the FDA has approved the following biologics for the treatment of colorectal cancer: AVASTIN®: ERBITUX® (cetuximab, ImClone Systems Inc., New York, N.Y., and Bristol-Myers Squibb, New York, N.Y.; is a monoclonal antibody directed against the epidermal growth factor receptor (EGFR)); GLEEVEC® (imatinib mesylate; a protein kinase inhibitor); and ERGAMISOL-K (levamisole hydrochloride, Janssen Pharmaceutica Products, LP, Titusville, N.J.; an immunomodulator approved by the FDA in 1990 as an adjuvant treatment in combination with 5-fluorouracil after surgical resection in patients with Dukes' Stage C colon cancer).

For the treatment of lung cancer, exemplary biologics include TARCEVA® (erlotinib HCL, OSI Pharmaceuticals Inc., Melville, N.Y.; a small molecule designed to target the human epidermal growth factor receptor 1 (HER1) pathway).

For the treatment of multiple myeloma, exemplary biologics include VELCADE® (bortezomib, Millennium Pharmaceuticals. Cambridge Mass.; a proteasome inhibitor). Additional biologics include THALIDOMID® (thalidomide, Clegene Corporation, Warren, N.J.; an immunomodulatory agent and appears to have multiple actions, including the ability to inhibit the growth and survival of myeloma cells and anti-angiogenesis).

Additional exemplary cancer therapeutic antibodies include, but are not limited to, 3F8, abagovomab, adecatumumab, afutuzumab, alacizumab pegol, alemtuzumab (CAMPATH®, MABCAMPATH®), altumomab pentetate (HYBRI-CEAKER®), anatumomab mafenatox, anrukinzumab (IMA-638), apolizumab, arcitumomab (CEA-SCAN®), bavituximab, bectumomab (LYMPHOSCAN®), belimumab (BENLYSTA®, LYMPHOSTAT-B®), besilesomab (SCINTIMUN®), bevacizumab (AVASTIN®), bivatuzumab mertansine, blinatumomab, brentuximab vedotin, cantuzumab mertansine, capromab pendetide (PROSTASCINT®), catumaxomab (REMOVAB®), CC49, cetuximab (C225, ERBITUX®), citatuzumab bogatox, cixutumumab, clivatuzumab tetraxetan, conatumumab, dacetuzumab, denosumab (PROLIA®), detumomab, ecromeximab, edrecolomab (PANOREX®), elotuzumab, epitumomab cituxetan, epratuzumab, ertumaxomab (REXOMUN®), etaracizumab, farletuzumab, figitumumab, fresolimumab, galiximab, gemtuzumab ozogamicin (MYLOTARG®), girentuximab, glembatumumab vedotin, ibritumomab (ibritumomab tiuxetan, ZEVALIN®), igovomab (INDIMACIS-125®), intetumumab, inotuzumab ozogamicin, ipilimumab, iratumumab, labetuzumab (CEA-CIDE®), lexatumumab, lintuzumab, lucatumumab, lumiliximab, mapatumumab, matuzumab, milatuzumab, minretumomab, mitumomab, nacolomab tafenatox, naptumomab estafenatox, necitumumab, nimotuzumab (THERACIM®, THERALOC®), nofetumomab merpentan (VERLUMA®), ofatumumab (ARZERRA®), olaratumab, oportuzumab monatox, oregovomab (OVAREX®), panitumumab (VECTIBIX®), pemtumomab (THERAGYN®), pertuzumab (OMNITARG®), pintumomab, pritumumab, ramucirumab, ranibizumab (LUCENTIS®), rilotumumab, rituximab (MABTHERA®, RITUXAN®), robatumumab, satumomab pendetide, sibrotuzumab, siltuximab, sontuzumab, tacatuzumab tetraxetan (AFP-CIDE®), taplitumomab paptox, tenatumomab, TGN1412, ticilimumab (tremelimumab), tigatuzumab, TNX-650, tositumomab (BEXXAR®), trastuzumab (HERCEPTIN®), tremelimumab, tucotuzumab celmoleukin, veltuzumab, volociximab, votumumab (HUMASPECT®), zalutumumab (HUMAX-EGFR®), and zanolimumab (HUMAX-CD4®).

In some embodiments, the multispecific or multifunctional molecules as described herein are administered in combination with a viral cancer therapeutic agent. Exemplary viral cancer therapeutic agents include, but not limited to, vaccinia virus (vvDD-CDSR), carcinoembryonic antigen-expressing measles virus, recombinant vaccinia virus (TK-deletion plus GM-CSF), Seneca Valley virus-001, Newcastle virus, coxsackie virus A21, GL-ONC1, EBNA1 C-terminal/LMP2 chimeric protein-expressing recombinant modified vaccinia Ankara vaccine, carcinoembryonic antigen-expressing measles virus, G207 oncolytic virus, modified vaccinia virus Ankara vaccine expressing p53, OncoVEX GM-CSF modified herpes-simplex 1 virus, fowlpox virus vaccine vector, recombinant vaccinia prostate-specific antigen vaccine, human papillomavirus 16/18 L1 virus-like particle/AS04 vaccine, MVA-EBNA1/LMP2 Inj. vaccine, quadrivalent HPV vaccine, quadrivalent human papillomavirus (types 6, 11, 16, 18) recombinant vaccine (GARDASIL®), recombinant fowlpox-CEA(6D)/TRICOM vaccine; recombinant vaccinia-CEA(6D)-TRICOM vaccine, recombinant modified vaccinia Ankara-5T4 vaccine, recombinant fowlpox-TRICOM vaccine, oncolytic herpes virus NV1020, HPV L1 VLP vaccine V504, human papillomavirus bivalent (types 16 and 18) vaccine (CERVARIX®), herpes simplex virus HF10, Ad5CMV-p53 gene, recombinant vaccinia DF3/MUC1 vaccine, recombinant vaccinia-MUC-1 vaccine, recombinant vaccinia-TRICOM vaccine, ALVAC MART-1 vaccine, replication-defective herpes simplex virus type 1 (HSV-1) vector expressing human Preproenkephalin (NP2), wild-type reovirus, reovirus type 3 Dearing (REOLYSIN®), oncolytic virus HSV1716, recombinant modified vaccinia Ankara (MVA)-based vaccine encoding Epstein-Barr virus target antigens, recombinant fowlpox-prostate specific antigen vaccine, recombinant vaccinia prostate-specific antigen vaccine, recombinant vaccinia-B7.1 vaccine, rAd-p53 gene, Ad5-delta24RGD. HPV vaccine 580299, JX-594 (thymidine kinase-deleted vaccinia virus plus GM-CSF), HPV-16/18 L1/AS04, fowlpox virus vaccine vector, vaccinia-tyrosinase vaccine, MEDI-517 HPV-16/18 VLP AS04 vaccine, adenoviral vector containing the thymidine kinase of herpes simplex virus TK99UN, HspE7, FP253/Fludarabine, ALVAC(2) melanoma multi-antigen therapeutic vaccine, ALVAC-hB7.1, canarypox-hIL-12 melanoma vaccine, Ad-REIC/Dkk-3, rAd-IFN SCH 721015, TIL-Ad-INFg, Ad-ISF35, and coxsackievirus A21 (CVA21, CAVATAK®).

In some embodiments, the multispecific or multifunctional molecules as described herein are administered in combination with a nanopharmaceutical. Exemplary cancer nanopharmaceuticals include, but not limited to, ABRAXANE® (paclitaxel bound albumin nanoparticles). CRLX101 (CPT conjugated to a linear cyclodextrin-based polymer), CRLX288 (conjugating docetaxel to the biodegradable polymer poly (lactic-co-glycolic acid)), cytarabine liposomal (liposomal Ara-C, DEPOCYT™), daunorubicin liposomal (DAUNOXOME®), doxorubicin liposomal (DOXIL®, CAELYX®), encapsulated-daunorubicin citrate liposome (DAUNOXOME®), and PEG anti-VEGF aptamer (MACUGEN,).

In some embodiments, the multispecific or multifunctional molecules as described herein are administered in combination with paclitaxel or a paclitaxel formulation, e.g., TAXOL®, protein-bound paclitaxel (e.g., ABRAXANE®). Exemplary paclitaxel formulations include, but are not limited to, nanoparticle albumin-bound paclitaxel (ABRAXANE®, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17:617-620).

Exemplary RNAi and antisense RNA agents for treating cancer include, but not limited to, CALAA-01, siG12D LODER (Local Drug EluteR), and ALN-VSP02.

Other cancer therapeutic agents include, but not limited to, cytokines (e.g., aldesleukin (IL-2, Interleukin-2, PROLEUKIN®), alpha Interferon (IFN-alpha, Interferon alfa, INTRON® A (Interferon alfa-2b), ROFERON-A®, (Interferon alfa-2a)), Epoetin alfa (PROCRIT®), filgrastim (G-CSF, Granulocyte-Colony Stimulating Factor, NEUPOGEN®), GM-CSF (Granulocyte Macrophage Colony Stimulating Factor, sargramostim, LEUKINE™), IL-11 (Interleukin-11, oprelvekin, NEUMEGA®), Interferon alfa-2b (PEG conjugate) (PEG interferon. PEG-INTRON™), and pegfilgrastim (NEULASTA™)), hormone therapy agents (e.g., aminoglutethimide (CYTADREN®), anastrozole (ARIMIDEX®), bicalutamide (CASODEX®), exemestane (AROMASIN®), fluoxymesterone (HALOTESTIN®), flutamide (EULEXIN®), fulvestrant (FASLODEX®,), goserelin (ZOLADEX®), letrozole (FEMARA®), leuprolide (ELIGARD™, LUPRON™, LUPRON DEPOT®, VIADUR™), megestrol (megestrol acetate, MEGACE®)), nilutamide (ANANDRON®, NILANDRON®), octreotide (octreotide acetate, SANDOSTATIN®, SANDOSTATIN LAR®,), raloxifene (EVISTA®), romiplostim (NPLATE®), tamoxifen (NOVALDEX®), and toremifene (FARESTON,)), phospholipase A2 inhibitors (e.g., anagrelide (AGRYLIN®)), biologic response modifiers (e.g., BCG (THERACYS®, TICE®), and Darbepoetin alfa (ARANESP®)), target therapy agents (e.g., bortezomib (VELCADE®), dasatinib (SPRYCEL™), denileukin diftitox (ONTAK®), erlotinib (TARCEVA®), everolimus (AFTNITOR®), gefitinib (IRESSA®), imatinib mesylate (STI-571, GLEEVEC™), lapatinib (TYKERB®), sorafenib (NEXAVAR®), and SU11248 (sunitinib, SUTENT(®)), immunomodulatory and antiangiogenic agents (e.g., CC-5013 (lenalidomide, REVLIMID®), and thalidomide (THALOMID®)), glucocorticosteroids (e.g., cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE), MEDRALONE®, MEDROL®, M-PREDNISOL®, SOLU-MEDROL®), prednisolone (DELTA-CORTEF(®, ORAPRED®, PEDIAPRED®, PRELONE®), and prednisone (DELTASONE®, LIQUID PRED®, METICORTEN®, ORASONE®)), and bisphosphonates (e.g., pamidronate (AREDIA®), and zoledronic acid (ZOMETA®)).

In some embodiments, the multispecific or multifunctional molecules as described herein are used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., an antibody against VEGF, a VEGF trap, a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-ß inhibitor)), a RAF-1 inhibitor, a KIT inhibitor and a RET inhibitor. In some embodiments, the anti-cancer agent used in combination with the AHCM agent is selected from the group consisting of: axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA(®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951(tivozanib), axitinib, BAY 73-4506 (regorafenib), brivanib alaninate (BMS-582664), brivanib (BMS-540215), cediranib (AZD2171), CHIR-258 (dovitinib), CP 673451. CYC116, E7080, Ki8751, masitinib (AB1010), MGCD-265, motesanib diphosphate (AMG-706), MP-470, OSI-930, Pazopanib Hydrochloride. PD173074, Sorafenib Tosylate (Bay 43-9006), SU 5402, TSU-68(SU6668), vatalanib, XL880 (GSK1363089, EXEL-2880). Selected tyrosine kinase inhibitors are chosen from sunitinib, erlotinib, gefitinib, or sorafenib. In some embodiments, the tyrosine kinase inhibitor is sunitinib.

In some embodiments, the multispecific or multifunctional molecules as described herein are administered in combination with one of more of: an anti-angiogenic agent, or a vascular targeting agent or a vascular disrupting agent. Exemplary anti-angiogenic agents include, but are not limited to, VEGF inhibitors (e.g., anti-VEGF antibodies (e.g., bevacizumab); VEGF receptor inhibitors (e.g., itraconazole); inhibitors of cell proliferatin and/or migration of endothelial cells (e.g., carboxyamidotriazole, TNP-470); inhibitors of angiogenesis stimulators (e.g., suramin), among others. A vascular-targeting agent (VTA) or vascular disrupting agent (VDA) is designed to damage the vasculature (blood vessels) of cancer tumors causing central necrosis (reviewed in, e.g., Thorpe, P. E. (2004) Clin. Cancer Res. Vol. 10:415-427). VTAs can be small-molecule. Exemplary small-molecule VTAs include, but are not limited to, microtubule destabilizing drugs (e.g., combretastatin A-4 disodium phosphate (CA4P), ZD6126, AVE8062, Oxi 4503); and vadimezan (ASA404).

### Immune Checkpoint Inhibitors

In other embodiments, methods described herein comprise use of an immune checkpoint inhibitor in combination with the multispecific or multifunctional molecules as described herein. The methods can be used in a therapeutic protocol in vivo.

In some embodiments, an immune checkpoint inhibitor inhibits a checkpoint molecule. Exemplary checkpoint molecules include but are not limited to CTLA4, PD1, PD-L1, PD-L2, TIM3, LAG3, CD160, 2B4, CD80, CD86, B7-H3 (CD276), B7-H4 (VTCN1), HVEM (TNFRSF14 or CD270), BTLA, KIR, MHC class I, MHC class II, GAL9, VISTA, BTLA, TIGIT, LAIR1, and A2aR. See, e.g., Pardoll. Nat. Rev. Cancer 12.4(2012):252-64, incorporated herein by reference.

In some embodiments, the immune checkpoint inhibitor is a PD-1 inhibitor, e.g., an anti-PD-1 antibody such as Nivolumab. Pembrolizumab or Pidilizumab. Nivolumab (also called MDX-1106, MDX-1106-04, ONO-4538, or BMS-936558) is a fully human IgG4 monoclonal antibody that specifically inhibits PD1. See, e.g., U.S. Pat. No. 8,008,449 and WO2006/121168. Pembrolizumab (also called Lambrolizumab, MK-3475, MK03475. SCH-900475 or KEYTRUDA(®: Merck) is a humanized IgG4 monoclonal antibody that binds to PD-1. See, e.g., Hamid, O. et al. (2013) New England Journal of Medicine 369 (2): 134-44, U.S. Pat. No. 8,354,509 and WO2009/114335. Pidilizumab (also called CT-0II or Cure Tech) is a humanized IgG1k monoclonal antibody that binds to PD1. See, e.g., WO2009/101611. In some embodiments, the inhibitor of PD-1 is an antibody molecule having a sequence substantially identical or similar thereto, e.g., a sequence at least 85%, 90%, 95% identical or higher to the sequence of Nivolumab, Pembrolizumab or Pidilizumab. Additional anti-PD1 antibodies, e.g., AMP 514 (Amplimmune), are described, e.g., in U.S. Pat. No. 8,609,089, US 2010028330, and/or US 20120114649.

In some embodiments, the immune checkpoint inhibitor comprises nivolumab or pembrolizumab. In some embodiments, the immune checkpoint inhibitor is an antibody that blocks the binding of human PD-L1 and human PD-L2 to hPD-1 (e.g., human PD-1). In some embodiments, the antibody blocking the binding of human PD-L1 and human PD-L2 to hPD-1 comprises three light chain CDRs having the amino acid sequences set forth in SEQ ID NOs: 15, 16 and 17. The sequence RASKGVSTSGYSYLH can comprise SEQ ID NO: 15. The sequence LASYLES can comprise SEQ ID NO: 16. The sequence QHSRDLPLT can comprise SEQ ID NO: 17. In some embodiments, the antibody blocking the binding of human PD-L1 and human PD-L2 to hPD-1 comprises three heavy chain CDRs having the amino acid sequences set forth in SEQ ID NOs: 18, 19 and 20. The sequence NYYMY can comprise SEQ ID NO: 18. The sequence GINPSNGGTNFNEKFKN can comprise SEQ ID NO: 19. The sequence RDYRFDMGFDY can comprise SEQ ID NO: 20.

The immune checkpoint inhibitor may be administered to a subject in need thereof to treat a disease or condition. In some embodiments, the immune checkpoint inhibitor may be administered at a dose of at least about 50 mg, at least about 75 mg, at least about 100 mg, at least about 150 mg, at least about 200 mg, at least about 225 mg, at least about 250 mg, at least about 275 mg, at least about 300 mg, at least about 325 mg, at least about 350 mg, at least about 375 mg, at least about 400 mg, at least about 425 mg, at least about 450 mg, at least about 475 mg, at least about 500 mg. In some embodiments, the immune checkpoint inhibitor may be administered at a dose of at most about 500 mg, at most about 475 mg, at most about 450 mg, at most about 425 mg, at most about 400 mg, at most about 375 mg, at most about 350 mg, at most about 325 mg, at most about 300 mg, at most about 275 mg, at most about 250 mg, at most about 225 mg, at most about 200 mg, at most about 150 mg, at most about 100 mg, at most about 75 mg, or at most about 50 mg. In some embodiments, the immune checkpoint inhibitor may be administered at a dose from about 100 mg to about 600 mg. In some embodiments, the immune checkpoint inhibitor may be administered at a dose from about 100 mg to about 150 mg, about 100 mg to about 200 mg, about 100 mg to about 225 mg, about 100 mg to about 250 mg, about 100 mg to about 300 mg, about 100 mg to about 350 mg, about 100 mg to about 400 mg, about 100 mg to about 450 mg, about 100 mg to about 500 mg, about 100 mg to about 550 mg, about 100 mg to about 600 mg, about 150 mg to about 200 mg, about 150 mg to about 225 mg, about 150 mg to about 250 mg, about 150 mg to about 300 mg, about 150 mg to about 350 mg, about 150 mg to about 400 mg, about 150 mg to about 450 mg, about 150 mg to about 500 mg, about 150 mg to about 550 mg, about 150 mg to about 600 mg, about 200 mg to about 225 mg, about 200 mg to about 250 mg, about 200 mg to about 300 mg, about 200 mg to about 350 mg, about 200 mg to about 400 mg, about 200 mg to about 450 mg, about 200 mg to about 500 mg, about 200 mg to about 550 mg, about 200 mg to about 600 mg, about 225 mg to about 250 mg, about 225 mg to about 300 mg, about 225 mg to about 350 mg, about 225 mg to about 400 mg, about 225 mg to about 450 mg, about 225 mg to about 500 mg, about 225 mg to about 550 mg, about 225 mg to about 600 mg, about 250 mg to about 300 mg, about 250 mg to about 350 mg, about 250 mg to about 400 mg, about 250 mg to about 450 mg, about 250 mg to about 500 mg, about 250 mg to about 550 mg, about 250 mg to about 600 mg, about 300 mg to about 350 mg, about 300 mg to about 400 mg, about 300 mg to about 450 mg, about 300 mg to about 500 mg, about 300 mg to about 550 mg, about 300 mg to about 600 mg, about 350 mg to about 400 mg, about 350 mg to about 450 mg, about 350 mg to about 500 mg, about 350 mg to about 550 mg, about 350 mg to about 600 mg, about 400 mg to about 450 mg, about 400 mg to about 500 mg, about 400 mg to about 550 mg, about 400 mg to about 600 mg, about 450 mg to about 500 mg, about 450 mg to about 550 mg, about 450 mg to about 600 mg, about 500 mg to about 550 mg, about 500 mg to about 600 mg, or about 550 mg to about 600 mg.

In some embodiments, the immune checkpoint inhibitor is administered every day, every 2, 3, 4, 5, or 6 days, every week, every 2, 3, 4, 5, 6, 7, or 8 weeks, or every 3, 4, 5, 6, 7, 8, 9, 10, 11, or 12 months.

In some embodiments, the immune checkpoint inhibitor is administered at a dose of at least about 100 mg, at least about 200 mg, at least about 300 mg, or at least about 400 mg every 3 weeks. In some embodiments, the immune checkpoint inhibitor is administered at a dose of 200 mg every 3 weeks. In some embodiments, the immune checkpoint inhibitor is administered at a dose of at least about 200 mg, at least about 300 mg, at least about 400 mg, at least about 500 mg, or at least about 600 mg every 6 weeks. In some embodiments, the immune checkpoint inhibitor is administered at a dose of 400 mg every 6 weeks.

In some embodiments, the immune checkpoint inhibitor is administered at a dose of at least about 100 mg, at least about 200 mg, at least about 300 mg, or at least about 400 mg every 2 weeks. In some embodiments, the immune checkpoint inhibitor is administered at a dose of 240 mg every 2 weeks. In some embodiments, the immune checkpoint inhibitor is administered at a dose of at least about 200 mg, at least about 300 mg, at least about 400 mg, at least about 500 mg, or at least about 600 mg every 4 weeks. In some embodiments, the immune checkpoint inhibitor is administered at a dose of 480 mg every 4 weeks.

In some embodiments, the immune checkpoint inhibitor is administered at a dose of at least about 1 mg/kg, at least about 2 mg/kg, at least about 2.5 mg/kg, at least about 3 mg/kg, at least about 3.5 mg/kg, at least about 4 mg/kg, at least about 4.5 mg/kg, at least about 5 mg/kg, at least about 5.5 mg/kg, or at least about 6 mg/kg body weight. In some embodiments, the immune checkpoint inhibitor is administered at a dose of at most about 6 mg/kg, at most about 5.5 mg/kg, at most about 5 mg/kg, at most about 4.5 mg/kg, at most about 4 mg/kg, at most about 3.5 mg/kg, at most about 3 mg/kg, at most about 2.5 mg/kg, at most about 2 mg/kg or at most about 1 mg/kg. In some embodiments, the immune checkpoint inhibitor is administered at a dose from about 1 mg/kg to about 8 mg/kg body weight. In some embodiments, the immune checkpoint inhibitor is administered at a dose from about 1 mg/kg to about 1.5 mg/kg, about 1 mg/kg to about 2 mg/kg, about 1 mg/kg to about 2.5 mg/kg, about 1 mg/kg to about 3 mg/kg, about 1 mg/kg to about 3.5 mg/kg, about 1 mg/kg to about 4 mg/kg, about 1 mg/kg to about 4.5 mg/kg, about 1 mg/kg to about 5 mg/kg, about 1 mg/kg to about 5.5 mg/kg, about 1 mg/kg to about 6 mg/kg, about 1 mg/kg to about 8 mg/kg, about 1.5 mg/kg to about 2 mg/kg, about 1.5 mg/kg to about 2.5 mg/kg, about 1.5 mg/kg to about 3 mg/kg, about 1.5 mg/kg to about 3.5 mg/kg, about 1.5 mg/kg to about 4 mg/kg, about 1.5 mg/kg to about 4.5 mg/kg, about 1.5 mg/kg to about 5 mg/kg, about 1.5 mg/kg to about 5.5 mg/kg, about 1.5 mg/kg to about 6 mg/kg, about 1.5 mg/kg to about 8 mg/kg, about 2 mg/kg to about 2.5 mg/kg, about 2 mg/kg to about 3 mg/kg, about 2 mg/kg to about 3.5 mg/kg, about 2 mg/kg to about 4 mg/kg, about 2 mg/kg to about 4.5 mg/kg, about 2 mg/kg to about 5 mg/kg, about 2 mg/kg to about 5.5 mg/kg, about 2 mg/kg to about 6 mg/kg, about 2 mg/kg to about 8 mg/kg, about 2.5 mg/kg to about 3 mg/kg, about 2.5 mg/kg to about 3.5 mg/kg, about 2.5 mg/kg to about 4 mg/kg, about 2.5 mg/kg to about 4.5 mg/kg, about 2.5 mg/kg to about 5 mg/kg, about 2.5 mg/kg to about 5.5 mg/kg, about 2.5 mg/kg to about 6 mg/kg, about 2.5 mg/kg to about 8 mg/kg, about 3 mg/kg to about 3.5 mg/kg, about 3 mg/kg to about 4 mg/kg, about 3 mg/kg to about 4.5 mg/kg, about 3 mg/kg to about 5 mg/kg, about 3 mg/kg to about 5.5 mg/kg, about 3 mg/kg to about 6 mg/kg, about 3 mg/kg to about 8 mg/kg, about 3.5 mg/kg to about 4 mg/kg, about 3.5 mg/kg to about 4.5 mg/kg, about 3.5 mg/kg to about 5 mg/kg, about 3.5 mg/kg to about 5.5 mg/kg, about 3.5 mg/kg to about 6 mg/kg, about 3.5 mg/kg to about 8 mg/kg, about 4 mg/kg to about 4.5 mg/kg, about 4 mg/kg to about 5 mg/kg, about 4 mg/kg to about 5.5 mg/kg, about 4 mg/kg to about 6 mg/kg, about 4 mg/kg to about 8 mg/kg, about 4.5 mg/kg to about 5 mg/kg, about 4.5 mg/kg to about 5.5 mg/kg, about 4.5 mg/kg to about 6 mg/kg, about 4.5 mg/kg to about 8 mg/kg, about 5 mg/kg to about 5.5 mg/kg, about 5 mg/kg to about 6 mg/kg, about 5 mg/kg to about 8 mg/kg, about 5.5 mg/kg to about 6 mg/kg, about 5.5 mg/kg to about 8 mg/kg, or about 6 mg/kg to about 8 mg/kg body weight.

In some embodiments, the immune checkpoint inhibitor is administered as an intravenous infusion over 10 minutes, 20 minutes, 30 minutes, 40 minutes, 50 minutes, 60 minutes, 90 minutes, 120 minutes, 150 minutes, 180 minutes, 4 hours, 5 hours, or 6 hours. In some embodiments, the immune checkpoint inhibitor may be administered Q3W or Q6W. In some embodiments, the anti-PD-1 antibody or antigen-binding portion thereof is administered via intravenous infusion at a dose of 200 mg on cycle day 1 every 3 weeks. In some embodiments, the anti-PD-1 antibody or antigen-binding portion thereof is administered via intravenous infusion at a dose of 400 mg on cycle day 1 every 6 weeks. In some embodiments, the anti-PD-1 antibody or antigen-binding portion thereof is administered via intravenous infusion at a dose of 240 mg on cycle day 1 every 2 weeks. In some embodiments, the anti-PD-1 antibody or antigen-binding portion thereof is administered via intravenous infusion at a dose of 480 mg on cycle day 1 every 4 weeks.

In some embodiments, an anti-PD-1 antibody or antigen-binding portion thereof may cross-compete with nivolumab for binding to human PD-1. In some embodiments, an anti-PD-1 antibody or antigen-binding portion thereof may cross-compete with pembrolizumab for binding to human PD-1. In some embodiments, an anti-PD-1 antibody or antigen-binding portion thereof may comprise a heavy chain constant region. In some embodiments, the heavy chain constant region is of a human IgG1 or IgG4 isotype. In some embodiments, an anti-PD-1 antibody or antigen-binding portion thereof is a chimeric, humanized or human monoclonal antibody or a portion thereof. The anti-PD-1 antibody may be nivolumab. The anti-PD-1 antibody may be pembrolizumab. In some embodiments, the anti-PD-1 antibody or antigen-binding portion thereof is administered at a dose ranging from 0.1 to 10.0 mg/kg body weight once every 2, 3 or 4 weeks. In some embodiments, the anti-PD-1 antibody or antigen-binding portion thereof is administered to a patient once every 2, 3, 4, 5, 6, 7, or 8 weeks. In some embodiments, the anti-PD-1 antibody or antigen-binding portion thereof is administered to a patient once every approximately 6 weeks. In some embodiments, the anti-PD-1 antibody or antigen-binding portion thereof is administered to a patient every six weeks, every six weeks ±5 days, ±4 days, ±3 days, ±2 days or ±1 day. In some embodiments, the immune checkpoint inhibitor may be administered to a subject on days 1 and 2 of a treatment cycle, days 1 and 3 of a treatment cycle, days 1 and 4 of a treatment cycle, days 1 and 5 of a treatment cycle, days 1 and 6 of a treatment cycle, days 1 and 7 of a treatment cycle, days 1 and 8 of a treatment cycle, days 1 and 9 of a treatment cycle, days 1 and 10 of a treatment cycle, days 1 and 11 of a treatment cycle, days 1 and 12 of a treatment cycle, days 1 and 13 of a treatment cycle, or days 1 and 14 of a treatment cycle.

In some embodiments, the immune checkpoint inhibitor may be administered to a subject for a total period of at least about 1 week, at least about 2 weeks, at least about 3 weeks, at least about 4 weeks, at least about 5 weeks, at least about 6 weeks, at least about 7 weeks, at least about 8 weeks, at least about 10 weeks, at least about 12 weeks, at least about 15 weeks, at least about 20 weeks, or more. In some embodiments, the immune checkpoint inhibitor may be administered to a subject for a total period of at most about 20 weeks, at most about 15 weeks, at most about 12 weeks, at most about 10 weeks, at most about 8 weeks, at most about 7 weeks, at most about 6 weeks, at most about 5 weeks, at most about 4 weeks, at most about 3 weeks, at most about 2 weeks, or at most about 1 week. In some embodiments, the immune checkpoint inhibitor may be administered to a subject for a total period from about 2 weeks to about 60 weeks. In some embodiments, the immune checkpoint inhibitor may be administered to a subject for a total period from about 2 weeks to about 4 weeks, about 2 weeks to about 8 weeks, about 2 weeks to about 12 weeks, about 2 weeks to about 15 weeks, about 2 weeks to about 20 weeks, about 2 weeks to about 25 weeks, about 2 weeks to about 30 weeks, about 2 weeks to about 36 weeks, about 2 weeks to about 40 weeks, about 2 weeks to about 50 weeks, about 2 weeks to about 60 weeks, about 4 weeks to about 8 weeks, about 4 weeks to about 12 weeks, about 4 weeks to about 15 weeks, about 4 weeks to about 20 weeks, about 4 weeks to about 25 weeks, about 4 weeks to about 30 weeks, about 4 weeks to about 36 weeks, about 4 weeks to about 40 weeks, about 4 weeks to about 50 weeks, about 4 weeks to about 60 weeks, about 8 weeks to about 12 weeks, about 8 weeks to about 15 weeks, about 8 weeks to about 20 weeks, about 8 weeks to about 25 weeks, about 8 weeks to about 30 weeks, about 8 weeks to about 36 weeks, about 8 weeks to about 40 weeks, about 8 weeks to about 50 weeks, about 8 weeks to about 60 weeks, about 12 weeks to about 15 weeks, about 12 weeks to about 20 weeks, about 12 weeks to about 25 weeks, about 12 weeks to about 30 weeks, about 12 weeks to about 36 weeks, about 12 weeks to about 40 weeks, about 12 weeks to about 50 weeks, about 12 weeks to about 60 weeks, about 15 weeks to about 20 weeks, about 15 weeks to about 25 weeks, about 15 weeks to about 30 weeks, about 15 weeks to about 36 weeks, about 15 weeks to about 40 weeks, about 15 weeks to about 50 weeks, about 15 weeks to about 60 weeks, about 20 weeks to about 25 weeks, about 20 weeks to about 30 weeks, about 20 weeks to about 36 weeks, about 20 weeks to about 40 weeks, about 20 weeks to about 50 weeks, about 20 weeks to about 60 weeks, about 25 weeks to about 30 weeks, about 25 weeks to about 36 weeks, about 25 weeks to about 40 weeks, about 25 weeks to about 50 weeks, about 25 weeks to about 60 weeks, about 30 weeks to about 36 weeks, about 30 weeks to about 40 weeks, about 30 weeks to about 50 weeks, about 30 weeks to about 60 weeks, about 36 weeks to about 40 weeks, about 36 weeks to about 50 weeks, about 36 weeks to about 60 weeks, about 40 weeks to about 50 weeks, about 40 weeks to about 60 weeks, or about 50 weeks to about 60 weeks.

In some embodiments, the anti-PD-1 antibody or antigen-binding portion thereof is administered at a dose of 1, 2, 3, 4, 5, or 6 mg/kg body weight once every 2 weeks. In some embodiments, the anti-PD-1 antibody or antigen-binding portion thereof is administered at a dose of 3 mg/kg body weight once every 2 weeks. In some embodiments, the anti-PD-1 antibody or antigen-binding portion thereof is administered at a dose of 3, 4, 5, 6, 7, 8, 9, or 10 mg/kg body weight once every 3 weeks. In some embodiments, the anti-PD-1 antibody or antigen-binding portion thereof is administered at a dose of 5 or 10 mg/kg body weight once every 3 weeks. In some embodiments, the anti-PD-1 antibody or antigen-binding portion thereof is administered at a dose of 2, 3, 4, 5, 6, 7, 8, 9, or 10 mg/kg body weight once every 4 weeks. In some embodiments, the anti-PD-1 antibody or antigen-binding portion thereof is administered at a dose of 4 to 12 mg/kg body weight once every 4 weeks. In some embodiments, the anti-PD-1 antibody or antigen-binding portion thereof is administered at a dose of 2, 3, 4, 5, 6, 7, 8, 9, or 10 mg/kg body weight once every 6 weeks. In some embodiments, the anti-PD-1 antibody or antigen-binding portion thereof is administered at a dose of 4 to 12 mg/kg body weight once every 6 weeks.

An immune checkpoint inhibitor may be administered in combination with the multifunctional molecule described herein. In some embodiments, the immune checkpoint inhibitor may be administered after the multifunctional molecule is administered. In some embodiments, the immune checkpoint inhibitor may be administered before the multifunctional molecule is administered. In some embodiments, the immune checkpoint inhibitor may be administered concurrently with administration of the multifunctional molecule.

Provided herein are methods of treating cancer in a human subject in need thereof comprising administering to the human subject a multifunctional molecule. In some embodiments, the method comprises administering to the human subject an immune checkpoint inhibitor prior to administering the multifunctional molecule. In some embodiments, the immune checkpoint inhibitor may be administered to a human subject during a period of at least about 1, at least about 2, at least about 3, at least about 4, at least about 5, at least about 6, at least about 7, at least about 8, at least about 9, at least about 10, at least about 12, at least about 15, or at least about 20 weeks in which the multifunctional molecule is administered. In some embodiments, the immune checkpoint inhibitor may be administered to a human subject during a period from about 1 week to about 52 weeks in which the multifunctional molecule is administered. In some embodiments, the immune checkpoint inhibitor may be administered to a human subject after a period of at least about 1, at least about 2, at least about 3, at least about 4, at least about 5, at least about 6, at least about 7, at least about 8, at least about 9, at least about 10, at least about 12, at least about 15, or at least about 20 weeks in which the multifunctional molecule is administered. In some embodiments, the immune checkpoint inhibitor may be administered to a human subject after a period from about 1 week to about 52 weeks in which the multifunctional molecule is administered.

In some embodiments, the multifunctional molecule described herein may be administered to a human subject for a total period of at least about 1 week, at least about 2 weeks, at least about 3 weeks, at least about 4 weeks, at least about 5 weeks, at least about 6 weeks, at least about 8 weeks, at least about 12 weeks, at least about 16 weeks, at least about 20 weeks, at least about 24 weeks, or at least about 36 weeks. In some embodiments, the multifunctional molecule described herein may be administered to a human subject for a total period of at most about 36 weeks, at most about 24 weeks, at most about 20 weeks, at most about 16 weeks, at most about 12 weeks, at most about 8 weeks, at most about 6 weeks, at most about 5 weeks, at most about 4 weeks, at most about 3 weeks, at most about 2 weeks, or at most about 1 week. In some embodiments, the multifunctional molecule described herein may be administered to a human subject for a total period from about 1 week to about 72 weeks. In some embodiments, the multifunctional molecule described herein may be administered to a human subject for a total period from about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 48 weeks, about 1 week to about 52 weeks, about 1 week to about 66 weeks, about 1 week to about 72 weeks, about 4 weeks to about 8 weeks, about 4 weeks to about 12 weeks, about 4 weeks to about 16 weeks, about 4 weeks to about 20 weeks, about 4 weeks to about 24 weeks, about 4 weeks to about 36 weeks, about 4 weeks to about 48 weeks, about 4 weeks to about 52 weeks, about 4 weeks to about 66 weeks, about 4 weeks to about 72 weeks, about 8 weeks to about 12 weeks, about 8 weeks to about 16 weeks, about 8 weeks to about 20 weeks, about 8 weeks to about 24 weeks, about 8 weeks to about 36 weeks, about 8 weeks to about 48 weeks, about 8 weeks to about 52 weeks, about 8 weeks to about 66 weeks, about 8 weeks to about 72 weeks, about 12 weeks to about 16 weeks, about 12 weeks to about 20 weeks, about 12 weeks to about 24 weeks, about 12 weeks to about 36 weeks, about 12 weeks to about 48 weeks, about 12 weeks to about 52 weeks, about 12 weeks to about 66 weeks, about 12 weeks to about 72 weeks, about 16 weeks to about 20 weeks, about 16 weeks to about 24 weeks, about 16 weeks to about 36 weeks, about 16 weeks to about 48 weeks, about 16 weeks to about 52 weeks, about 16 weeks to about 66 weeks, about 16 weeks to about 72 weeks, about 20 weeks to about 24 weeks, about 20 weeks to about 36 weeks, about 20 weeks to about 48 weeks, about 20 weeks to about 52 weeks, about 20 weeks to about 66 weeks, about 20 weeks to about 72 weeks, about 24 weeks to about 36 weeks, about 24 weeks to about 48 weeks, about 24 weeks to about 52 weeks, about 24 weeks to about 66 weeks, about 24 weeks to about 72 weeks, about 36 weeks to about 48 weeks, about 36 weeks to about 52 weeks, about 36 weeks to about 66 weeks, about 36 weeks to about 72 weeks, about 48 weeks to about 52 weeks, about 48 weeks to about 66 weeks, about 48 weeks to about 72 weeks, about 52 weeks to about 66 weeks, about 52 weeks to about 72 weeks, or about 66 weeks to about 72 weeks.

The methods described herein may comprise administering to the human subject the multifunctional molecule on days 1 and 8 of a treatment cycle, on days 1 and 16 of a treatment cycle, on days 1 and 24 of a treatment cycle, on days 1 and 32 of a treatment cycle, on days 1 and 40 of a treatment cycle, or on days 1 and 48 of a treatment cycle. In some embodiments, a period of 1 day, 2 days, 3 days, 4 days, 5 days, 6 days, 7 days, 14 days, 21 days, 30 days, or more may occur between doses of the multifunctional molecule.

In some embodiments, the method comprises administering to the human subject the immune checkpoint inhibitor at least about 1, 2, 3, 4, 5, 6, 7, 8, 9, 10, 11, 12, or more times prior to administering the multifunctional molecule. In some embodiments, the method comprises administering to the human subject the immune checkpoint inhibitor at least about 1, 2, 3, 4, 5, 6, 7, 8, 9, 10, 11, 12, or more times after administering the multifunctional molecule.

A human subject administered the multifunctional molecule may be a patient. In some embodiments, the patient may have a tumor with high PD-L 1 expression [e.g., Tumor Proportion Score (TPS)>50%] and/or was not previously treated with platinum-containing chemotherapy. In some embodiments, the patient may have a tumor with high PD-L1 expression [e.g., Tumor Proportion Score (TPS)>50%] and/or was previously treated with platinum-containing chemotherapy. In some embodiments, a patient's tumor may have no EGFR and/or ALK genomic aberrations.

The methods described herein may further comprise administering a platinum-based chemotherapy to a human subject in need thereof. In some embodiments, the methods described herein may further comprise administering pemetrexed to the patient. In some embodiments, the methods described herein may further comprise administering carboplatin to the patient. In some embodiments, the methods described herein may further comprise administering pemetrexed and carboplatin to the patient. In some embodiments, the method comprises administering to the human subject a platinum-based chemotherapy prior to administering the multifunctional molecule. In some embodiments, the method comprises administering to the human subject a platinum-based chemotherapy after administering the multifunctional molecule. In some embodiments, the method comprises administering to the human subject an immune checkpoint inhibitor and a platinum-based chemotherapy prior to administering the multifunctional molecule. In some embodiments, the method comprises administering to the human subject an immune checkpoint inhibitor and a platinum-based chemotherapy after administering the multifunctional molecule. In some embodiments, the method comprises administering to the human subject an immune checkpoint inhibitor and/or a platinum-based chemotherapy prior for a period of at least about 1, at least about 2, at least about 3, at least about 4, at least about 5, at least about 6, at least about 7, at least about 8, at least about 9, at least about 10, or at least about 12 weeks prior to administering the multifunctional molecule. In some embodiments, the method comprises administering to the human subject an immune checkpoint inhibitor and/or a platinum-based chemotherapy prior for a period of at least about 1, at least about 2, at least about 3, at least about 4, at least about 5, at least about 6, at least about 7, at least about 8, at least about 9, at least about 10, or at least about 12 weeks after administering the multifunctional molecule.

In some embodiments, a platinum-based chemotherapy may not be administered to a human subject during or after administration of the multifunctional molecule. In some embodiments, a platinum-based chemotherapy may not be administered to a human subject before administration of the multifunctional molecule. In some embodiments, a platinum-based chemotherapy may not be administered to the human subject following administration of the immune checkpoint inhibitor and/or a platinum-based chemotherapy for a period of at least about 1, at least about 2, at least about 3, at least about 4, at least about 5, at least about 6, at least about 7, at least about 8, at least about 9, at least about 10, or at least about 12 weeks prior to administering the multifunctional molecule.

A platinum-based chemotherapy may be a platinum-based doublet chemotherapy (PT-DC). In some embodiments, the PT-DC may be a combination of pemetrexed and carboplatin. In some embodiments, the PT-DC may be administered concurrently with an anti-PD-1 antibody or antigen-binding portion thereof. In some embodiments, the PT-DC may be administered concurrently with an anti-PD-1 antibody or antigen-binding portion thereof for at least 1, at least 2, at least 3, or at least 4 doses of the anti-PD-1 antibody or antigen-binding portion thereof. In some embodiments, the PT-DC may be administered concurrently with an anti-PD-1 antibody or antigen-binding portion thereof for at least 4 doses of the anti-PD-1 antibody or antigen-binding portion thereof, followed by repeated administration of the anti-PD-1 antibody or antigen-binding portion thereof alone.

A patient may have nonsquamous non-small cell lung cancer. In some embodiments, the patient may have nonsquamous non-small cell lung cancer and a dose of pemetrexed may be administered to the patient. In some embodiments, the patient may have nonsquamous non-small cell lung cancer and pemetrexed may be administered to the patient in an amount of at least about 100 mg/m², at least about 200 mg/m², at least about 300 mg/m2, at least about 400 mg/m², at least about 500 mg/m², at least about 600 mg/m², at least about 700 mg/m2, or at least about 800 mg/m² about every 21 days. In some embodiments, the patient may have nonsquamous non-small cell lung cancer and pemetrexed may be administered to the patient in an amount of 500 mg/m² about every 21 days.

In some embodiments, the methods described herein may further comprise administering a dose of folic acid to the patient at least once per day. In some embodiments, a dose of folic acid may be at least about 200 pg, at least about 300 pg, at least about 400 pg, at least about 500 pg, at least about 600 pg, at least about 700 pg, at least about 800 μg per day. In some embodiments, the methods described herein may further comprise administering from about 400 μg to about 1000 μg of folic acid to the patient once per day, beginning about 7 days prior to administering pemetrexed to the patient. In some embodiments, the methods described herein may further comprise administering from about 400 μg to about 1000 μg of folic acid to the patient once per day, beginning about 7 days prior to administering pemetrexed to the patient, beginning about 7 days prior to administering pemetrexed to the patient and continuing until about 21 days after the patient is administered the last dose of pemetrexed.

In some embodiments, the methods described herein may further comprise administering a dose of vitamin B₁₂ to the patient. In some embodiments, the dose of vitamin B₁₂ may be at least about 0.5 mg, at least about 1 mg, at least about 1.5 mg, at least about 2 mg, at least about 5 mg, or at least about 10 mg. In some embodiments, the methods described herein may further comprise administering about 1 mg of vitamin B₁₂ to the patient at least about 1 week prior to the first administration of pemetrexed. In some embodiments, the methods described herein may further comprise administering about 1 mg of vitamin B₁₂ to the patient about every three cycles of pemetrexed administration. In some embodiments, the methods described herein may further comprise administering about 1 mg of vitamin B₁₂ to the patient at least about 1 week prior to the first administration of pemetrexed and about every three cycles of pemetrexed administration.

In some embodiments, the methods described herein may further comprise administering dexamethasone to the patient twice a day on the day before, the day of, and the day after pemetrexed administration.

In some embodiments, the PD-1 inhibitor is an immunoadhesin, e.g., an immunoadhesin comprising an extracellular/PD-1 binding portion of a PD-1 ligand (e.g., PD-L1 or PD-L2) that is fused to a constant region (e.g., an Fc region of an immunoglobulin). In some embodiments, the PD-1 inhibitor is AMP-224 (B7-DCIg, e.g., described in WO2011/066342 and WO2010/027827), a PD-L2 Fc fusion soluble receptor that blocks the interaction between B7-H1 and PD-1.

In some embodiments, the immune checkpoint inhibitor is a PD-L1 inhibitor, e.g., an antibody molecule. In some embodiments, the PD-L1 inhibitor is YW243.55.S70, MPDL3280A, MEDI-4736, MSB-00I0718C, or MDX-1105. In some embodiments, the anti-PD-L1 antibody is MSB0010718C (also called A09-246-2; Merck Serono), which is a monoclonal antibody that binds to PD-L 1. Exemplary humanized anti-PD-L1 antibodies are described, e.g., in WO2013/079174. In some embodiments, the PD-L1 inhibitor is an anti-PD-L1 antibody, e.g., YW243.55.570. The YW243.55.570 antibody is described, e.g., in WO 2010/077634. In some embodiments, the PD-L1 inhibitor is MDX-1105 (also called BMS-936559), which is described, e.g., in WO2007/005874. In some embodiments, the PD-L1 inhibitor is MDPL3280A (Genentech/Roche), which is a human Fc-optimized IgG1 monoclonal antibody against PD-L1. See, e.g., U.S. Pat. No. 7,943,743 and U.S Publication No.: 20120039906. In some embodiments, the inhibitor of PD-L1 is an antibody molecule having a sequence substantially identical or similar thereto, e.g., a sequence at least 85%, 90%, 95% identical or higher to the sequence of YW243.55.S70. MPDL3280A, MEDI-4736, MSB-0010718C. or MDX-1105.

In some embodiments, the immune checkpoint inhibitor is a PD-L2 inhibitor, e.g., AMP-224 (which is a PD-L2 Fc fusion soluble receptor that blocks the interaction between PD1 and B7-H1. See, e.g., WO2010/027827 and WO2011/066342.

In some embodiments, the immune checkpoint inhibitor is a LAG-3 inhibitor, e.g., an anti LAG-3 antibody molecule. In some embodiments, the anti-LAG-3 antibody is BMS-986016 (also called BMS986016: Bristol-Myers Squibb). BMS-986016 and other humanized anti-LAG-3 antibodies are described, e.g., in US 2011/0150892, WO2010/019570, and WO2014/008218.

In some embodiments, the immune checkpoint inhibitor is a TIM-3 inhibitor, e.g., anti-TIM3 antibody molecule. e.g., described in U.S. Pat. No. 8,552,156. WO 2011/155607, EP 2581113 and U.S Publication No.: 2014/044728.

In some embodiments, the immune checkpoint inhibitor is a CTLA-4 inhibitor, e.g., anti-CTLA-4 antibody molecule. Exemplary anti-CTLA4 antibodies include Tremelimumab (igG2 monoclonal antibody from Pfizer, formerly known as ticilimumab, CP-675,206); and Ipilimumab (also called MDX-010, CAS No. 477202-00-9). Other exemplary anti-CTLA-4 antibodies are described, e.g., in U.S. Pat. No. 5,811,097.

### Method of Expanding Cells

Any of the compositions and methods described herein can be used to expand an immune cell population. An immune cell provided herein includes an immune cell derived from a hematopoietic stem cell or an immune cell derived from a non-hematopoietic stem cell, e.g., by differentiation or de-differentiation.

An immune cell includes a hematopoietic stem cell, progeny thereof and/or cells that have differentiated from said HSC, e.g., lymphoid cells or myeloid cells. An immune cell can be an adaptive immune cell or an innate immune cell. Examples of immune cells include T cells, B cells, Natural Killer cells, Natural Killer T cells, neutrophils, dendritic cells, monocytes, macrophages, and granulocytes.

In some embodiments, an immune cell is a T cell. In some embodiments, a T cell includes a CD4+ T cell, a CD8+ T cell, a TCR alpha-beta T cell, a TCR gamma-delta T cell. In some embodiments, a T cell comprises a memory T cell (e.g., a central memory T cell, or an effector memory T cell (e.g., a TEMRA) or an effector T cell. In some embodiments, a T cell comprises a tumor infiltrating lymphocyte (TIL).

In some embodiments, an immune cell is an NK cell.

In some embodiments, an immune cell is a TIL. TILs are immune cells (e.g., T cells, B cells or NK cells) that can be found in a tumor or around a tumor (e.g., in the stroma or tumor microenvironment of a tumor), e.g., a solid tumor, e.g., as described herein. TILs can be obtained from a sample from a subject having cancer, e.g., a biopsy or a surgical sample. In some embodiments, TILs can be expanded using a method as described herein. In some embodiments, a population of expanded TILs, e.g., expanded using a method as described herein, can be administered to a subject to treat a disease, e.g., a cancer.

In some embodiments, immune cells, e.g., T cells (e.g., TILs), can be obtained from a unit of blood collected from a subject using any number of techniques known to the skilled artisan, such as Ficoll™ separation. In one aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations. The methods described herein can include more than one selection step, e.g., more than one depletion step.

In one embodiment, the methods as described herein can utilize culture media conditions comprising DMEM, DMEM F12, RPMI 1640, and/or AIM V media. The media can be supplemented with glutamine, HEPES buffer (e.g., 10 mM), serum (e.g., heat-inactivated serum, e.g., 10%), and/or beta mercaptocthanol (e.g., 55 uM). In some embodiments, the culture conditions as described herein comprise one or more supplements, cytokines, growth factors, or hormones. In some embodiments, the culture condition comprises one or more of IL-2, IL-15, or IL-7, or a combination thereof.

Immune effector cells such as T cells may be activated and expanded generally using methods as described, for example, in U.S. Pat. Nos. 6,352,694; 6,534,055; or 6,905,680. Generally, a population of immune cells, may be expanded by contact with an agent that stimulates a CD3/TCR complex associated signal and a ligand that stimulates a costimulatory molecule on the surface of the T cells; and/or by contact with a cytokine, e.g., IL-2, IL-15 or IL-7. T cell expansion protocols can also include stimulation, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besangon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, a TIL population can also be expanded by methods known in the art. For example, a population of TILs can be expanded as described in Hall et al., Journal for ImmunoTherapy of Cancer (2016) 4:61, the entire contents of which are hereby incorporated by reference. Briefly, TILs can be isolated from a sample by mechanical and/or physical digestion. The resultant TIL population can be stimulated with an anti-CD3 antibody in the presence of non-dividing feeder cells. In some embodiments, the TIL population can be cultured, e.g., expanded, in the presence of IL-2, e.g., human IL-2. In some embodiments, the TIL cells can be cultured, e.g., expanded for a period of at least 1-21 days, e.g., at least 1, 2, 3, 4, 5, 6, 7, 8, 9, 10, 11, 12, 13, 14, 15, 16, 17, 18, 19, 20 or 21 days.

As described herein, in some embodiments, an immune cell population (e.g., a T cell (e.g., a TEMRA cell or a TIL population) can be expanded by contacting the immune cell population with the multifunctional polypeptide molecule as described herein.

In some embodiments, the expansion occurs in vivo, e.g., in a subject. In some embodiments, a subject is administered the multifunctional polypeptide molecule as described herein resulting in expansion of immune cells in vivo.

In some embodiments, the expansion occurs ex vivo, e.g., in vitro. In some embodiments, cells from a subject, e.g., T cells, e.g., TIL cells, are expanded in vitro with the multifunctional polypeptide molecule as described herein. In some embodiments, the expanded TILs are administered to the subject to treat a disease or a symptom of a disease.

In some embodiments, a method of expansion as described herein results in an expansion of at least 1.1-10 fold, 10-20 fold, or 20-50 fold expansion. In some embodiments, the expansion is at least 1.1, 1.2, 1.3, 1.4, 1.5, 2, 3, 4, 5, 6, 7, 8, 9, 10, 15, 20, 25, 30, 35, 40, 45, 50, 55, 60, 65, 70, 75, 80, 85, 90, 100, 200, 300, 400, 500, 600, 700, 800, 900, 1000, 1500, 2000, 2500, 3000, 3500, 400, 4500, 5000, 5500, 6000, 6500, 7000, 7500, 8000, 8500, 9000, 9500, or 10000 fold expansion.

In some embodiments, a method of expansion as described herein comprises culturing, e.g., expanding, the cells for at least about 4 hours, 6 hours, 10 hours, 12 hours, 15 hours, 18 hours, 20 hours, or 22 hours. In some embodiments, a method of expansion as described herein comprises culturing, e.g., expanding, the cells for at least 1, 2, 3, 4, 5, 6, 7, 8, 9, 10, 11, 12, 13, 14, 15, 16, 17, 18, 19, 20 or 21 days. In some embodiments, a method of expansion as described herein comprises culturing, e.g., expanding, the cells for at least about 1 week, 2 weeks, 3 weeks, 4 weeks, 5 weeks, 6 weeks, 7 weeks or 8 weeks.

In some embodiments, a method of expansion as described herein is performed on immune cells obtained from a healthy subject.

In some embodiments, a method of expansion as described herein is performed on immune cells (e.g., TILs) obtained from a subject having a disease. e.g., a cancer, e.g., a solid tumor as described herein.

In some embodiments, a method of expansion as described herein further comprises contacting the population of cells with an agent, that promotes, e.g., increases, immune cell expansion. In some embodiments, the agent comprises an immune checkpoint inhibitor, e.g., a PD-1 inhibitor, a LAG-3 inhibitor, a CTLA4 inhibitor, or a TIM-3 inhibitor. In some embodiments, the agent comprises a 4-1BB agonist, e.g., an anti-4-1BB antibody.

Without wishing to be bound by theory, in some embodiments, the multifunctional polypeptide molecule as described herein can expand, e.g., selectively or preferentially expand, T cells expressing a T cell receptor (TCR) comprising a TCR alpha and/or TCR beta molecule, e.g., TCR alpha-beta T cells (αβ T cells). In some embodiments, the multifunctional polypeptide molecule as described herein does not expand, or induce proliferation of T cells expressing a TCR comprising a TCR gamma and/or TCR delta molecule, e.g., TCR gamma-delta T cells (γδ T cells). In some embodiments, the multifunctional polypeptide molecule as described herein selectively or preferentially expands as T cells over γδ T cells.

Without wishing to be bound by theory, in some embodiments, γδ T cells are associated with cytokine release syndrome (CRS) and/or neurotoxicity (NT). In some embodiments, the multispecific or multifunctional molecules as described herein result in selective expansion of non-γδ T cells, e.g., expansion of αβ T cells, thus reducing CRS and/or NT.

In some embodiments, any of the compositions or methods as described herein result in an immune cell population having a reduction of, e.g., depletion of, γδ T cells. In some embodiments, the immune cell population is contacted with an agent that reduces, e.g., inhibits or depletes, yS T cells, e.g., an anti-IL-17 antibody or an agent that binds to a TCR gamma and/or TCR delta molecule.

In some embodiments, the multifunctional polypeptide molecule as described herein results in expansion of an immune cell, e.g., a T cell, a tumor infiltrating lymphocyte (TIL), an NK cell, or other immune cells (e.g., as described herein).

In some embodiments, binding of the multifunctional polypeptide molecule as described herein to a TCRβV region results in one, two, three or all of: (i) reduced T cell proliferation kinetics; (ii) cell killing, e.g., target cell killing, e.g. cancer cell killing, e.g., as measured by an assay of Example 4; (iii) increased Natural Killer (NK) cell proliferation, e.g., expansion; or (iv) expansion, e.g., at least about 1.1-10 expansion (e.g., at least about 1.1, 1.2, 1.3, 1.4, 1.5, 2, 3, 4, 5, 6, 7, 8, 9, or 10 fold expansion), of a population of T cells having a memory-like phenotype, e.g., as described herein, e.g., wherein (i)-(iv) are relative to the non-TCRβV-binding T cell engager.

In some embodiments, the method further comprises contacting the population of cells with an agent that promotes, e.g., increases, immune cell expansion. In some embodiments, the agent includes an immune checkpoint inhibitor, e.g., as described herein. In some embodiments, the agent includes a 4-1BB (CD127) agonist, e.g., an anti-4-1BB antibody.

In some embodiments, the method further comprises comprising contacting the population of cells with a non-dividing population of cells, e.g., feeder cells, e.g., irradiated allogenic human PBMCs.

In some embodiments, expansion of the population of immune cells, is compared to expansion of a similar population of cells with an antibody that binds to: a CD3 molecule, e.g., CD3 epsilon (CD3e) molecule: or a TCR alpha (TCRα) molecule.

In some embodiments, expansion of the population of immune cells, is compared to expansion of a similar population of cells not contacted with the anti-TCRβV antibody molecule or the multispecific or multifunctional molecules as described herein.

In some embodiments, expansion of the population of memory effector T cells, e.g., TEM cells, e.g., TEMRA cells, is compared to expansion of a similar population of cells with an antibody that binds to: a CD3 molecule, e.g., CD3 epsilon (CD3e) molecule; or a TCR alpha (TCRα) molecule.

In some embodiments, the method results in expansion of, e.g., selective or preferential expansion of, T cells expressing a T cell receptor (TCR) comprising a TCR alpha and/or TCR beta molecule, e.g., TCR alpha-beta T cells (ap T cells).

In some embodiments, the method results in expansion of αβT cells overexpansion of T cells expressing a TCR comprising a TCR gamma and/or TCR delta molecule, e.g., TCR gamma-delta T cells (γδ T cells). In some embodiments, expansion of γδ T cells over γδ T cells results in reduced production of cytokines associated with CRS. In some embodiments, expansion of αβT cells over γδ T cells results in immune cells that have reduced capacity to, e.g., are less prone to, induce CRS upon administration into a subject.

In some embodiments, an immune cell population (e.g., T cells (e.g., TEMRA cells or TILs) or NK cells) cultured in the presence of, e.g., expanded with, the multifunctional polypeptide molecule as described herein does not induce CRS and/or NT when administered into a subject, e.g., a subject having a disease or condition as described herein.

In some embodiments, provided herein is a multifunctional polypeptide molecule as described herein comprising a non-murine, e.g., a human-like antibody molecule (e.g., a human or humanized antibody molecule), which binds, e.g., specifically binds, to a T cell receptor beta variable (TCRβV) region. In some embodiments, binding of the multifunctional polypeptide molecule as described herein results in expansion, e.g., at least about 1.1-50 fold expansion (e.g., at least about 1.540 fold, 2-35 fold, 3-30 fold, 5-25 fold, 8-20 fold, or 10-15 fold expansion), of a population of T cells, e.g., a population of T cells having a memory-like phenotype, e.g., CD45RA+ CCR7− T cells. In some embodiments, the population of T cells having a memory-like phenotype comprises CD4+ and/or CD8+ T cells. In some embodiments, the population of T cells having a memory-like phenotype comprises a population of memory T cells, e.g., T effector memory (TEM) cells, e.g., TEM cells expressing CD45RA (TEMRA) cells, e.g., CD4+ or CD8+ TEMRA cells. In some embodiments, the population of T cells having a memory-like phenotype does not express a senescent marker, e.g., CD57. In some embodiments, the population of T cells having a memory-like phenotype does not express an inhibitory receptor, e.g., OX40, 4-1BB, and/or ICOS.

In some embodiments, the population of T cells having a memory-like phenotype is a population of T cells with CD45RA+ CCR7−CD57−. In some embodiments, the population of T cells having a memory-like phenotype does not express an inhibitory receptor, e.g., OX40, 4-1BB, and/or ICOS.

In some embodiments, the population of T cells having a memory-like phenotype, e.g., as described herein, has increased proliferative capacity, e.g., as compared to a reference cell population, e.g., an otherwise similar population of cells that has not been contacted with an anti-TCRβV antibody or the multispecific or multifunctional molecules as described herein.

In some embodiments, the expansion is at least about 1.1-10 fold expansion (e.g., at least about 1.1, 1.2, 1.3, 1.4, 1.5, 2, 3, 4, 5, 6, 7, 8, 9, or 10 fold expansion).

In some embodiments, expansion of the population of T cells having a memory-like phenotype, e.g., memory effector T cells, e.g., TEM cells, e.g., TEMRA cells, e.g., CD4+ or CD8+ TEMRA cells, is compared to expansion of a similar population of cells with an antibody that binds to: a CD3 molecule, e.g., CD3 epsilon (CD3e) molecule; or a TCR alpha (TCRα) molecule.

In some embodiments, the population of expanded T cells having a memory-like phenotype, e.g., T effector memory cells, comprises cells T cells, e.g., CD3+, CD8+ or CD4+ T cells. In some embodiments, the population of expanded T cells having a memory-like phenotype, T effector memory cells, comprises CD3+ and CD8+ T cells. In some embodiments, the population of expanded T cells having a memory-like phenotype, e.g., T effector memory cells comprises CD3+ and CD4+ T cells.

In some embodiments, the population of expanded T cells having a memory-like phenotype, T effector memory (TEM) cells, comprises cells T cells, e.g., CD3+, CD8+ or CD4+ T cells, which express or re-express, CD45RA, e.g., CD45RA+. In some embodiments, the population comprises TEM cells expressing CD45RA, e.g., TEMRA cells. In some embodiments, expression of CD45RA on TEMRA cells, e.g., CD4+ or CD8+ TEMRA cells, can be detected by a method as described herein. e.g., flow cytometry.

In some embodiments, the population of T cells having a memory-like phenotype, e.g., TEMRA cells have low or no expression of CCR7, e.g., CCR7− or CCR7 low. In some embodiments, expression of CCR7 on TEMRA cells cannot be detected by a method as described herein, e.g., flow cytometry.

In some embodiments, the population of T cells having a memory-like phenotype, e.g., TEMRA cells express CD95, e.g., CD95+. In some embodiments, expression of CD95 on TEMRA cells can be detected by a method as described herein. e.g., flow cytometry.

In some embodiments, the population of T cells having a memory-like phenotype, e.g., TEMRA cells express CD45RA, e.g., CD45RA+, have low or no expression of CCR7, e.g., CCR7− or CCR7 low, and express CD95, e.g., CD95+. In some embodiments, the population of T cells having a memory-like phenotype, e.g., TEMRA cells can be identified as CD45RA+, CCR7− and CD95+ cells. In some embodiments, the population of T cells having a memory-like phenotype, e.g., TEMRA cells comprise CD3+, CD4+ or CD8+ T cells (e.g., CD3+ T cells, CD3+CD8+ T cells, or CD3+CD4+ T cells).

In some embodiments, the population of T cells having a memory-like phenotype does not express a senescent marker, e.g., CD57.

In some embodiments, the population of T cells having a memory-like phenotype does not express an inhibitory receptor, e.g., OX40, 4-1BB, and/or ICOS.

In some embodiments, binding of the multifunctional polypeptide molecule as described herein results in expansion, e.g., at least about 1.1-50 fold expansion (e.g., at least about 1.5-40 fold, 2-35 fold, 3-30 fold, 5-25 fold, 8-20 fold, or 10-15 fold expansion), of a subpopulation of T cells. In some embodiments, the multifunctional polypeptide molecule as described herein-activated (e.g., expanded) subpopulation of T cells resemble TEMRA cells in high expression of CD45RA and/or low expression of CCR7. In some embodiments, the multifunctional polypeptide molecule as described herein-activated (e.g., expanded) subpopulation of T cells do not display upregulation of the senescence markers CD57 and/or KLRG1. In some embodiments, the multifunctional polypeptide molecule as described herein-activated (e.g., expanded) subpopulation of T cells do not display upregulation of co-stimulatory molecules CD27 and/or CD28. In some embodiments, the multifunctional polypeptide molecule as described herein-activated (e.g., expanded) subpopulation of T cells are highly proliferative. In some embodiments, the multifunctional polypeptide molecule as described herein-activated (e.g., expanded) subpopulation of T cells secrete IL-2. In some embodiments, expression of surface markers on T cells can be detected by a method as described herein, e.g., flow cytometry. In some embodiments, the proliferative capability of T cells can be detected by a method as described herein, e.g., a method described in Example 4. In some embodiments, cytokine expression of T cells can be detected by a method as described herein, e.g., a method described in Examples 10 and 35. In some embodiments, the expansion is at least about 1.1-10 fold expansion (e.g., at least about 1.1, 1.2, 1.3, 1.4, 1.5, 2, 3, 4, 5, 6, 7, 8, 9, or 10 fold expansion). In some embodiments, the expansion is compared to expansion of a similar population of cells with an antibody that binds to a CD3 molecule, e.g., CD3 epsilon (CD3e) molecule; or a TCR alpha (TCRα) molecule.

In some embodiments, binding of the multifunctional polypeptide molecule as described herein results in proliferation, e.g., expansion, e.g., at least about 1.1-50 fold expansion (e.g., at least about 1.5-40 fold, 2-35 fold, 3-30 fold, 5-25 fold, 8-20 fold, or 10-15 fold expansion), of a population of Natural Killer (NK) cells. In some embodiments, the expansion of NK cells is at least about 1.1-30 fold expansion (e.g., at least about 1.1, 1.2, 1.3, 1.4, 1.5, 2, 3, 4, 5, 6, 7, 8, 9, 10, 15, 20, 25, or at least about 1.1-5, 5-10, 10-15, 15-20, 20-25, or 25-30 fold expansion). In some embodiments, the expansion of NK cells is measure by an assay of Example 4. In some embodiments, the expansion of NK cells by, e.g., binding of, the multifunctional polypeptide molecule as described herein is compared to expansion of an otherwise similar population not contacted with the multifunctional polypeptide molecule as described herein.

In some embodiments, binding of the multifunctional polypeptide molecule as described herein results in cell killing, e.g., target cell killing, e.g. cancer cell killing. In some embodiments, the cancer cell is a hematological cancer cell or a solid tumor cell. In some embodiments, the cancer cell is a multiple myeloma cell. In some embodiments, binding of the multifunctional polypeptide molecule as described herein results in cell killing in vitro or in vivo. In some embodiments, cell killing is measured by an assay of Example 4.

In some embodiments, binding of the multifunctional polypeptide molecule as described herein to a TCRβV region results in an increase or decrease of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) of any of the activities described herein compared the activity of 16G8 or TM23 murine antibody, or a humanized version thereof as described in U.S. Pat. No. 5,861,155.

In some embodiments, the method comprises expanding, e.g., increasing the number of, an immune cell population in the subject. In some embodiments, provided herein is a method of expanding, e.g., increasing the number of, an immune cell population comprising, contacting the immune cell population with an effective amount of the multifunctional polypeptide molecule as described herein. In some embodiments, the expansion occurs in vivo or ex vivo (e.g., in vitro).

In some embodiments, provided herein is a method of expanding, e.g., increasing the number of, an immune cell population comprising, contacting the immune cell population with a multifunctional polypeptide molecule as described herein comprising an antibody molecule, e.g., humanized antibody molecule, which binds, e.g., specifically binds, to a T cell receptor beta variable chain (TCRβ 4V) region (e.g., anti-TCRβV antibody molecule), thereby expanding the immune cell population. In some embodiments, the expansion occurs in vivo or ex vivo (e.g., in vitro).

In some embodiments, provided herein is a method of expanding a population of immune effector cells from a subject having a cancer, the method comprising: (i) isolating a biological sample comprising a population of immune effector cells from the subject: e.g., a peripheral blood sample, biopsy sample, or bone marrow sample; (ii) acquiring a value of the status of one or more TCRβV molecules for the subject, e.g., in the biological sample from the subject, wherein said value comprises a measure of the presence of, e.g., level or activity of, a TCRβV molecule in a sample from the subject compared to a reference value, e.g., a sample from a health subject, wherein a value that is higher, e.g., increased, in the subject relative to the reference, e.g., healthy subject, is indicative of the presence of cancer in the subject, and (iii) contacting the biological sample comprising a population of immune effector cells with the multifunctional polypeptide molecule as described herein.

In some embodiments, the method further comprises administering the population of immune effector cells contacted with the multifunctional polypeptide molecule as described herein to the subject.

In some embodiments, a higher, e.g., increased, level or activity of one or more TCRβV molecules in a subject, e.g., in a sample from a subject, is indicative of a bias, e.g., a prferential expansion, e.g., clonal expansion, of T cells expressing said one or more TCRβV molecules in the subject.

Accordingly, provided herein are, inter alia, multispecific or multifunctional molecules comprising TCRβV-binding moieties as described herein (e.g., multispecific or multifunctional antibody molecules) that comprise anti-TCRβV antibody molecules, nucleic acids encoding the same, methods of producing the aforesaid molecules, pharmaceutical compositions comprising aforesaid molecules, and methods of treating a disease or disorder, e.g., cancer, using the aforesaid molecules. The antibody molecules and pharmaceutical compositions as described herein can be used (alone or in combination with other agents or therapeutic modalities) to treat, prevent and/or diagnose disorders and conditions, e.g., cancer, e.g., as described herein.

### Exemplary Multifunctional Polypeptide Molecule

Any of the compositions and methods described herein can be used to expand an immune cell population. An immune cell provided herein includes an immune cell derived from a hematopoietic stem cell or an immune cell derived from a non-hematopoietic stem cell, e.g., by differentiation or de-differentiation.

In some embodiments, the IL-2 molecule or a functional fragment or a functional variant thereof or the IL-2 C125A mutant molecule or a functional fragment or a functional variant thereof is operatively linked to the immunoglobulin heavy chain constant region via a linker.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, and a sequence having at least 50%, 55%, 60/₀, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide comprising the sequence of SEQ ID NO: 1346, and the sequence of SEQ ID NO: 3649; (ii) a second polypeptide comprising the sequence of SEQ ID NO: 2270, and the sequence of SEQ ID NO; 3648; and (iii) a third polypeptide comprising the sequence of SEQ ID NO: 1349, and the sequence of SEQ ID NO: 3644.

In some embodiments, the multifunctional polypeptide molecule comprises: (i) a first polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346 operatively linked to a sequence having at least 50%, 55%, 60%, 65%, 70%, 75%, 80%, 85%, 90%, 95%, 97%, 98%, 99%, 99.5%, 99.6%, 99.7% 99.8%, or 99.9% sequence identity to the sequence of SEQ ID NO: 3649; (ii) a second polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide comprising, from the N-terminus to the C-terminus, the sequence of SEQ ID NO: 1346 operatively linked to the sequence of SEQ ID NO: 3649; (ii) a second polypeptide comprising, from the N-terminus to the C-terminus, the sequence of SEQ ID NO: 2270 operatively linked to the sequence of SEQ ID NO: 3648; and (iii) a third polypeptide comprising, from the N-terminus to the C-terminus, the sequence of SEQ ID NO: 1349 operatively linked to the sequence of SEQ ID NO: 3644.

In some embodiments, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270 is operatively linked to a sequence having at least 50%, 55/o, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, or a combination thereof.

In some embodiments, the sequence of SEQ ID NO: 2270 is operatively linked to the sequence of SEQ ID NO: 3648 via the sequence of SEQ ID NO: 3308, or a combination thereof.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3517, 4000, 4004, 4006, 4008, 4010, 4011, 4014, 4016 or 4018; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3521, 4002, 4030, 4007, 4003, 4013 or 4015; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3518, 4005, 4009, 4012 or 4017.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3517; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3521; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3518.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4000; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4002; and (iii) a third polypeptide comprising a sequence having at least 50%, 55/₀, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3518.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4004; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4030; and (iii) a third polypeptide comprising a sequence having at least 50%, 55%, 60%, 65%, 70%, 75/a, 80%, 85%, 90%, 95%, 97%, 98%, 99%, 99.5%, 99.6%, 99.7%, 99.8%, or 99.9% sequence identity to the sequence of SEQ ID NO: 4005.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4006; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4007; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4005.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4008; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3521; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4009.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4010; (ii) a second polypeptide comprising a sequence having at least 50%, 55%, 60%, 65%, 70%, 75%, 80%, 85%, 90%, 95%, 97%, 98%, 99% 99.5%, 99.6%, 99.7%, 99.8%, or 99.9% sequence identity to the sequence of SEQ ID NO: 4003; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4009.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4011; (ii) a second polypeptide comprising a sequence having at least 50%, 55%, 60%, 65%, 70%, 75%, 80%, 85%, 90%, 95%, 97%, 98%, 99% 99.5%, 99.6%, 99.7%, 99.8%, or 99.9% sequence identity to the sequence of SEQ ID NO: 4013; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4012.

In some embodiments, the multifunctional polypeptide molecule comprises: (i) a first polypeptide comprising a sequence having at least 50%, 55%, 60%, 65%, 70%, 75%, 80%, 85/0, 90%, 95%, 97%, 98%, 99%, 99.5%, 99.6%, 99.7%, 99.8%, or 99.9% sequence identity to the sequence of SEQ ID NO: 4014; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4015; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4012.

In some embodiments, the multifunctional polypeptide molecule comprises: (i) a first polypeptide comprising a sequence having at least 50%, 55%, 60%, 65%, 70%, 75%, 80%, 85/0, 90%, 95%, 97%, 98%, 99%, 99.5%, 99.6%, 99.7%, 99.8%, or 99.9% sequence identity to the sequence of SEQ ID NO: 4016; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3521; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4017.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4018; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4002; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4017.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4019, 4021, 4023, 4025 or 4027; and (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4020, 4022, 4024, 4026 or 4028.

In some embodiments, the multifunctional polypeptide molecule comprises: (i) a first polypeptide comprising a sequence having at least 50%, 55%, 60%, 65%, 70%, 75%, 80%, 85/a, 90%, 95%, 97%, 98%, 99%, 99.5%, 99.6%, 99.7%, 99.8%, or 99.9% sequence identity to the sequence of SEQ ID NO: 4019; and (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4020.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4021; and (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4022.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4023; and (ii) a second polypeptide comprising a sequence having at least 50/₀, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4024.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4025; and (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4026.

In some embodiments, the multifunctional polypeptide molecule comprises: (i) a first polypeptide comprising a sequence having at least 50%, 55%, 60%, 65%, 70%, 75%, 80%, 85/₀, 90%, 95%, 97%, 98%, 99%, 99.5%, 99.6%, 99.7%, 99.8%, or 99.9% sequence identity to the sequence of SEQ ID NO: 4027; and (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 4028.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3530, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3531: (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2191, and a sequence having at least 50%,/₀, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3533; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3527, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3528.

In some embodiments, the multifunctional polypeptide molecule comprises: (i) a first polypeptide comprising the sequence of SEQ ID NO: 3530, and the sequence of SEQ ID NO: 3531; (ii) a second polypeptide comprising the sequence of SEQ ID NO: 2191, and the sequence of SEQ ID NO; 3533; and (iii) a third polypeptide comprising the sequence of SEQ ID NO: 3527, and the sequence of SEQ ID NO: 3528.

In some embodiments, the multifunctional polypeptide molecule comprises: (i) a first polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3530 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3531; (ii) a second polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2191 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3533; and (iii) a third polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3527 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3528.

In some embodiments, the multifunctional polypeptide molecule comprises: (i) a first polypeptide comprising, from the N-terminus to the C-terminus, the sequence of SEQ ID NO: 3530 operatively linked to the sequence of SEQ ID NO: 3531; (ii) a second polypeptide comprising, from the N-terminus to the C-terminus, the sequence of SEQ ID NO: 2191 operatively linked to the sequence of SEQ ID NO: 3533; and (iii) a third polypeptide comprising, from the N-terminus to the C-terminus, the sequence of SEQ ID NO: 3527 operatively linked to the sequence of SEQ ID NO: 3528.

In some embodiments, a sequence having at least 50%, 55%, 60%, 65%, 70%, 75%, 80%, 85%, 90%, 95%, 97%, 98%, 99%, 99.5%, 99.6%, 99.7% 99.8%, or 99.9% sequence identity to the sequence of SEQ ID NO: 2191 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3533 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, or a combination thereof.

In some embodiments, the sequence of SEQ ID NO: 2191 is operatively linked to the sequence of SEQ ID NO: 3533 via the sequence of SEQ ID NO: 3308, or a combination thereof.

In some embodiments, the first polypeptide further comprises a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3547 operatively linked to a sequence having at least 50%, 55%, 60%, 65%, 70%, 75%, 80%, 85%, 90%, 95%, 97%, 98%, 99%/a, 99.5%, 99.6%, 99.7%, 99.8%, or 99.9% sequence identity to the sequence of SEQ ID NO: 3531, the second polypeptide further comprises a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3534 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3533, or a combination thereof.

In some embodiments, the first polypeptide further comprises the sequence of SEQ ID NO: 3547 operatively linked to the sequence of SEQ ID NO: 3531, the second polypeptide further comprises the sequence of SEQ ID NO: 3534 operatively linked to the sequence of SEQ ID NO: 3533, or a combination thereof.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3529 or the sequence of SEQ ID NO: 3548, (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3532 or the sequence of SEQ ID NO: 3549; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3526.

In some embodiments, the multifunctional polypeptide molecule comprises: (i) a first polypeptide comprising the sequence of SEQ ID NO: 3529 or the sequence of SEQ ID NO: 3548: (ii) a second polypeptide comprising the sequence of SEQ ID NO: 3532 or the sequence of SEQ ID NO: 3549; and (iii) a third polypeptide comprising to the sequence of SEQ ID NO: 3526.

In some embodiments, the multifunctional polypeptide molecule comprises: (i) a first polypeptide comprising a sequence having at least 50%, 55%, 60/, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3530 and a sequence having at least 50%, 55%, 60%, 65%, 70%, 75/a, 80%, 85%, 90%, 95%, 97%, 98%, 99%, 99.5%, 99.6%, 99.7%, 99.8%, or 99.9% sequence identity to the sequence of SEQ ID NO: 3537; and (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3527, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3528, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2191.

In some embodiments, the multifunctional polypeptide molecule comprises: (i) a first polypeptide comprising the sequence of SEQ ID NO: 3530 and the sequence of SEQ ID NO: 3537; and (ii) a second polypeptide comprising the sequence of SEQ ID NO: 3527, the sequence of SEQ ID NO: 3528, and the sequence of SEQ ID NO: 2191.

In some embodiments, the multifunctional polypeptide molecule comprises: (i) a first polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3530 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3537; and (ii) a second polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3527 operatively linked to a sequence having at least 50%, 55%, 60%, 65%, 70%, 75%, 80%, 85%, 90%, 95%, 97%, 98%, 99%, 99.5/, 99.6%, 99.7%, 99.8%, or 99.9% sequence identity to the sequence of SEQ ID NO: 3528 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2191.

In some embodiments, the multifunctional polypeptide molecule comprises; (i) a first polypeptide comprising, from the N-terminus to the C-terminus, the sequence of SEQ ID NO: 3530 operatively linked to the sequence of SEQ ID NO: 3537; and (ii) a second polypeptide comprising, from the N-terminus to the C-terminus, the sequence of SEQ ID NO: 3527 operatively linked to the sequence of SEQ ID NO: 3528 operatively linked to the sequence of SEQ ID NO: 2191.

In some embodiments, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3528 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2191 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3309.

In some embodiments, the sequence of SEQ ID NO: 3528 is operatively linked to the sequence of SEQ ID NO: 2191 via the sequence of SEQ ID NO: 3309.

In some embodiments, the multifunctional polypeptide molecule comprises two first polypeptides and two second polypeptides.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3536; and (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3535.

In some embodiments, the multifunctional polypeptide molecule comprises: (i) a first polypeptide comprising the sequence of SEQ ID NO: 3536; and (ii) a second polypeptide comprising the sequence of SEQ ID NO: 3535.

In some embodiments, the multifunctional polypeptide molecule comprises: (i) a first polypeptide comprising one or more components as listed in Table 21; and (ii) a second polypeptide comprising one or more components as listed in Table 21. In some embodiments, the multifunctional polypeptide molecule further comprises: (i) a third polypeptide comprising one or more components as listed in Table 21; and (ii) a fourth polypeptide comprising one or more components as listed in Table 21.

In some embodiments, the multifunctional polypeptide molecule comprises: (i) a first polypeptide comprising one or more components comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the component sequences as listed in Table 21; and (ii) a second polypeptide comprising one or more components comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the component sequences as listed in Table 21. In some embodiments, the multifunctional polypeptide molecule further comprises: (i) a third polypeptide comprising one or more components comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the component sequences as listed in Table 21; and (ii) a fourth polypeptide comprising one or more components comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the component sequences as listed in Table 21.

In some embodiments, the multifunctional polypeptide molecule comprises: (i) a first polypeptide comprising one or more component sequences as listed in Table 21; and (ii) a second polypeptide comprising one or more component sequences as listed in Table 21. In some embodiments, the multifunctional polypeptide molecule further comprises: (i) a third polypeptide comprising one or more component sequences as listed in Table 21; and (ii) a fourth polypeptide comprising one or more component sequences as listed in Table 21.

In some embodiments, the multifunctional polypeptide molecule comprises: (i) a first polypeptide comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the polypeptide sequences as listed in Table 21; and (ii) a second polypeptide comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the polypeptide sequences as listed in Table 21. In some embodiments, the multifunctional polypeptide molecule further comprises: (i) a third polypeptide comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the polypeptide sequences as listed in Table 21; and (ii) a fourth polypeptide comprising an amino acid sequence having at least 50%, 55%, 60%, 65%, 70%, 75%, 80%, 85%, 90°/, 95%, 97%, 98%, 99%, 99.5%, 99.6%, 99.7%, 99.8%, or 99.9% sequence identity to any one of the polypeptide sequences as listed in Table 21.

In some embodiments, the multifunctional polypeptide molecule comprises: (i) a first polypeptide comprising any one of the polypeptide sequences as listed in Table 21; and (ii) a second polypeptide comprising any one of the polypeptide sequences as listed in Table 21. In some embodiments, the multifunctional polypeptide molecule further comprises: (i) a third polypeptide comprising any one of the polypeptide sequences as listed in Table 21; and (ii) a fourth polypeptide comprising one or more comprising any one of the polypeptide sequences as listed in Table 21.

In another aspect, described herein is an antibody comprising an anti-T cell receptor beta variable chain (TCRβV) binding domain comprising: (i) a heavy chain variable region (VH) comprising a heavy chain complementarity determining region 1 (HC CDR 1), a heavy chain complementarity determining region 2 (HC CDR2), and a heavy chain complementarity determining region 3 (HC CDR3) comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to SEQ ID NO: 3650, SEQ ID NO: 3651, and SEQ ID NO: 5, respectively; (ii) a light chain variable region (VL) comprising a light chain complementarity determining region 1 (LC CDR 1), a light chain complementarity determining region 2 (LC CDR2), and a light chain complementarity determining region 3 (LC CDR3) comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to SEQ ID NO: 3655, SEQ ID NO: 3653, and SEQ ID NO: 8, respectively; or (iii) a combination thereof.

In some embodiments, the TCRβV binding domain comprising: (i) a VH comprising a HC CDR 1, a HC CDR2, and a HC CDR3 comprising the amino acid sequence of SEQ ID NO: 3650, SEQ ID NO: 3651, and SEQ ID NO: 5, respectively: (ii) a VL comprising a LC CDR1, a LC CDR2, and a LC CDR3 comprising the amino acid sequence of SEQ ID NO: 3655, SEQ ID NO: 3653, and SEQ ID NO: 8, respectively; or (iii) a combination thereof.

In some embodiments, the TCRβV binding domain comprising: (i) a VH comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to SEQ ID NO: 1346; (ii) a VL comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to SEQ ID NO: 1349; or (iii) a combination thereof.

In some embodiments, the TCRβV binding domain comprising: (i) a VH comprising the amino acid sequence of SEQ ID NO: 1346; (ii) a VL comprising the amino acid sequence of SEQ ID NO: 1349; or (iii) a combination thereof.

In some embodiments, the antibody comprising an anti-T cell receptor beta variable chain (TCRβV) binding domain further comprises a cytokine polypeptide or a functional fragment or a functional variant thereof. In some embodiments, the cytokine polypeptide may be IL-2, IL2-C125A. In embodiments, the cytokine polypeptide may further comprise a cytokine receptor. In some embodiments, the cytokine polypeptide may comprise a cytokine dimer.

TABLE 1
Amino acid and nucleotide sequences for murine, chimeric and humanized antibody molecules
which bind to TCRVB 6, e.g., TCRVB 6-5. The antibody molecules include murine mAb Antibody A,
and humanized mAb Antibody A-H Clones A-H.1 to A-H.85. The amino acid the heavy and
light chain CDRs, and the amino acid and nucleotide sequences of the heavy and light
chain variable regions, and the heavy and light chains are shown.
Antibody A (murine), also referred to as H131, TCRVB 6-5 binder

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 45 | HC CDR1 (Kabat) | TYYIH |
| SEQ ID NO: 46 | HC CDR2 (Kabat) | WFFPGSGNIKYNEKFKG |
| SEQ ID NO: 47 | HC CDR3 (Kabat) | SYYSYDVLDY |
| SEQ ID NO: 48 | HC CDR1 (Chothia) | GYSFTTY |
| SEQ ID NO: 49 | HC CDR2 (Chothia) | FPGSGN |
| SEQ ID NO: 50 | HC CDR3 (Chothia) | SYYSYDVLDY |
| SEQ ID NO: 1 | VH | QVQLQQSGPELVKPGTSVKISCKASGYSFTTYYIHWVKQRPG |
| | | QGLEWIGWFFPGSGNIKYNEKFKGKATLTADTSSSTAYMQLS |
| | | SLTSEESAVYFCAGSYYSYDVLDYWGHGTTLTVSS |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: S1 | LC CDR1 (Kabat) | KASQNVGINVV |
| SEQ ID NO: 52 | LC CDR2 (Kabat) | SSSHRYS |
| SEQ ID NO: 53 | LC CDR3 (Kabat) | QQFKSYPLT |
| SEQ ID NO: 54 | LC CDR1 (Chothia) | KASQNVGINVV |
| SEQ ID NO: 55 | LC CDR2 (chothia) | SSSHRYS |
| SEQ ID NO: 56 | LC CDR3 (chothia) | QQFKSYPLT |
| SEQ ID NO: 2 | VL | DILMTQSQKFMSTSLGDRVSVSCKASQNVGINVVWHQQKPG |
| | | QSPKALIYSSSHRYSGVPDRFTGSGSGTDFTLTINNVQSED |
| | | LAEYFCQQFKSYPLTFGAGTKLELK |

Antibody A humanized (A-H antibody), TCRVB 6-5 binder
A-H.1 antibody (also referred to as BHM1709)

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 9 | VH | QVQLVQSGAEVKKPGSSVKVSCKASGYSFTTYYIHWVRQAP |
| | | GQGLEWMGWFFPGSGNIKYNEKFKGRVTITADTSTSTAYME |
| | | LSSLRSEDTAVYYCAGSYYSYDVLDYWGQGTTVTVSS |
| SEQ ID NO: 12 | DNA VH | CAGGTGCAGCTGGTTCAGTCTGGCGCCGAAGTGAAGAAAC |
| | | CTGGCTCCTCCGTGAAGGTGTCCTGCAAGGCTTCCGGCTAC |
| | | TCCTTCACCACCTACTACATCCACTGGGTCCGACAGGCCCC |
| | | TGGACAAGGATTGGAATGGATGGGCTGGTTCTTCCCCGGC |
| | | TCCGGCAACATCAAGTACAACGAGAAGTTCAAGGGCCGCG |
| | | TGACCATCACCGCCGACACCTCTACCTCTACCGCCTACATG |
| | | GAACTGTCCAGCCTGAGATCTGAGGACACCGCCGTGTACT |
| | | ACTGCGCCGGCTCCTACTACTCTTACGACGTGCTGGATTAC |
| | | TGGGGCCAGGGCACCACAGTGACAGTGTCCTCT |
| SEQ ID NO: 69 | VH-IgM | METDTLLLWVLLLWVPGSTGQVQLVQSGAEVKKPGSSVKVS |
| | constant delta | CKASGYSFTTYYIHWVRQAPGQGLEWMGWFFPGSGNIKYNE |
| | CDC | KFKGRVTITADTSTSTAYMELSSLRSEDTAVYYCAGSYYSYD |
| | | VLDYWGQGTTVTVSSGSASAPTLFPLVSCENSPSDTSSVAVG |
| | | CLAQDFLPDSITFSWKYKNNSDISSTRGFPSVLRGGKYAATSQ |
| | | VLLPSKDVMQGTDEHVVCKVQHPNGNKEKNVPLPVIAELPP |
| | | KVSVFVPPRDGFFGNPRKSKLICQATGFSPRQIQVSWLREGKQ |
| | | VGSGVTTDQVQAEAKESGPTTYKVTSTLTIKESDWLGQSMFT |
| | | CRVDHRGLTFQQNASSMCVPDQDTAIRVFAIPPSFASIFLTKS |
| | | TKLTCLVTDLTTYDSVTISWTRQNGEAVKTHTNISESHPNATF |
| | | SAVGEASICEDDWNSGERFTCTVTHTDLASSLKQTISRPKGVA |
| | | LHRPDVYLLPPAREQLNLRESATITCLVTGFSPADVFVQWMQ |
| | | RGQPLSPEKYVTSAPMPEPQAPGRYFAHSILTVSEEEWNTGET |
| | | YTCVVAHEALPNRVTERTVDKSTGKPTLYNVSLVMSDTAGT |
| | | CY |
| SEQ ID NO: 70 | VH-IgGA1 | METDTLLLWVLLLWVPGSTGQVQLVQSGAEVKKPGSSVKVS |
| | | CKASGYSFTTYYIHWVRQAPGQGLEWMGWFFPGSGNIKYNE |
| | | KFKGRVTITADTSTSTAYMELSSLRSEDTAVYYCAGSYYSYD |
| | | VLDYWGQGTTVTVSSASPTSPKVFPLSLCSTQPDGNVVIACL |
| | | VQGFFPQEPLSVTWSESGQGVTARNFPPSQDASGDLYTTSSQ |
| | | LTLPATQCLAGKSVTCHVKHYTNPSQDVTVPCPVPSTPPTPSP |
| | | STPPTPSPSCCHPRLSLHRPALEDLLLGSEANLTCTLTGLRDAS |
| | | GVTFTWTPSSGKSAVQGPPERDLCGCYSVSSVLPGCAEPWNH |
| | | GKTFTCTAAYPESKTPLTATLSKSGNTFRPEVHLLPPPSEELAL |
| | | NELVTLTCLARGFSPKDVLVRWLQGSQELPREKYLTWASRQ |
| | | EPSQGTTTFAVTSILRVAAEDWKKGDTFSCMVGHEALPLAFT |
| | | QKTIDRLAGKPTHVNVSVVMAEVDGTCY |
| SEQ ID NO: 71 | VH-IgGA2 | METDTLLLWVLLLWVPGSTGQVQLVQSGAEVKKPGSSVKVS |
| | | CKASGYSFTTYYIHWVRQAPGQGLEWMGWFFPGSGNIKYNE |
| | | KFKGRVTITADTSTSTAYMELSSLRSEDTAVYYCAGSYYSYD |
| | | VLDYWGQGTTVTVSSASPTSPKVFPLSLDSTPQDGNVVVACL |
| | | VQGFFPQEPLSVTWSESGQNVTARNFPPSQDASGDLYTTSSQ |
| | | LTLPATQCPDGKSVTCHVKHYTNSSQDVTVPCRVPPPPPCCH |
| | | PRLSLHRPALEDLLLGSEANLTCTLTGLRDASGATFTWTPSSG |
| | | KSAVQGPPERDLCGCYSVSSVLPGCAQPWNHGETFTCTAAHP |
| | | ELKTPLTANITKSGNTFRPEVHLLPPPSEELALNELVTLTCL |
| | | ARGFSPKDVLVRWLQGSQELPREKYLTWASRQEPSQGTTTYA |
| | | VTSILRVAAEDWKKGETFSCMVGHEALPLAFTQKTIDRMAG |
| | | KPTHINVSVVMAEADGTCY |
| SEQ ID NO: | Heavy chain | METDTLLLWVLLLWVPGSTGQVQLVQSGAEVKKPGSSVKVS |
| 3278 | | CKASGYSFTTYYIHWVRQAPGQGLEWMGWFFPGSGNIKYNE |
| | | KFKGRVTITADTSTSTAYMELSSLRSEDTAVYYCAGSYYSYD |
| | | VLDYWGQGTTVTVSSASTKGPSVFPLAPSSKSTSGGTAALGC |
| | | LVKDYFPEPVTVSWNSGALTSGVHTFPAVLQSSGLYSLSSVV |
| | | TVPSSSLGTQTYICNVNHKPSNTKVDKRVEPKSCDKTHTCPP |
| | | CPAPELLGGPSVFLFPPKPKDTLMISRTPEVTCVVVDVSHED |
| | | PEVKFNWYVDGVEVHNAKTKPREEQYNSTYRVVSVLTVLHQ |
| | | DWLNGKEYKCKVSNKALPAPIEKTISKAKGQPREPQVYTLPP |
| | | SREEMTKNQVSLTCLVKGFYPSDIAVEWESNGQPENNYKTTP |
| | | PVLDSDGSFFLYSKLTVDKSRWQQGNVFSCSVMHEALHNHY |
| | | TQKSLSLSPGK |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 10 | VL | DIQMTQSPSFLSASVGDRVTITCKASQNVGINVVWHQQKPGK |
| | | APKALIYSSSHRYSGVPSRFSGSGSGTEFTLTISSLQPEDFATYF |
| | | CQQFKSYPLTFGQGTKLEIK |
| SEQ ID NO: 13 | DNA VL | GACATCCAGATGACCCAGTCTCCATCCTTCCTGTCCGCCTC |
| | | TQTGGGCGACAGAGTGACCATCACATGCAAGGCCTCTCAG |
| | | AACGTGGGCATCAACGTCGTGTGGCACCAGCAGAAGCCTG |
| | | GCAAGGCTCCTAAGGCTCTGATCTACTCCTCCAGCCACCG |
| | | GTACTCTGGCGTGCCCTCTAGATTTTCCGGCTCTGGCTCTG |
| | | GCACCGAGTTTACCCTGACAATCTCCAGCCTGCAGCCTGA |
| | | GGACTTCGCCACCTACTTTTGCCAGCAGTTCAAGAGCTACC |
| | | CTCTGACCTTTGGCCAGGGCACCAAGCTGGAAATCAAG |
| SEQ ID NO: 72 | VL and kappa constant | METDTLLLWVLLLWVPGSTGDIQMTQSPSFLSASVGDRVTIT |
| | region/light chain | CKASQNVGINVVWHQQKPGKAPKALIYSSSHRYSGVPSRFSG |
| | | SGSGTEFTLTISSLQPEDFATYFCQQFKSYPLTFGQGTKLEIKR |
| | | TVAAPSVFIFPPSDEQLKSGTASVVCLLNNFYPREAKVQWKV |
| | | DNALQSGNSQESVTEQDSKDSTYSLSSTLTLSKADYEKHKVY |
| | | ACEVTHQGLSSPVTKSENRGEC |

A-H.2 antibody (also referred to as BHM1710)

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 9 | VH | QVQLVQSGAEVKKPGSSVKVSCKASGYSFTTYYIHWVRQAP |
| | | GQGLEWMGWFFPGSGNIKYNEKFKGRVTITADTSTSTAYME |
| | | LSSLRSEDTAVYYCAGSYYSYDVLDYWGQGTTVTVSS |
| SEQ ID NO: 12 | DNA VH | CAGGTGCAGCTGGTTCAGTCTGGCGCCGAAGTGAAGAAAC |
| | | CTGGCTCCTCCGTGAAGGTGTCCTGCAAGGCTTCCGGCTAC |
| | | TCCTTCACCACCTACTACATCCACTGGGTCCGACAGGCCCC |
| | | TGGACAAGGATTGGAATGGATGGGCTGGTTCTTCCCCGGC |
| | | TCCGGCAACATCAAGTACAACGAGAAGTTCAAGGGCCGCG |
| | | TGACCATCACCGCCGACACCTCTACCTCTACCGCCTACATG |
| | | GAACTGTCCAGCCTGAGATCTGAGGACACCGCCGTGTACT |
| | | ACTGCGCCGGCTCCTACTACTCTTACGACGTGCTGGATTAC |
| | | TGGGGCCAGGGCACCACAGTGACAGTGTCCTCT |
| SEQ ID NO: | Heavy chain | METDTLLLWVLLLWVPGSTGQVQLVQSGAEVKKPGSSVKVS |
| 3278 | | CKASGYSFTTYYIHWVRQAPGQGLEWMGWFFPGSGNIKYNE |
| | | KFKGRVTITADTSTSTAYMELSSLRSEDTAVYYCAGSYYSYD |
| | | VLDYWGQGTTVTVSSASTKGPSVFPLAPSSKSTSGGTAALGC |
| | | LVKDYFPEPVTVSWNSGALTSGVHTFPAVLQSSGLYSLSSVV |
| | | TVPSSSLGTQTYICNVNHKPSNTKVDKRVEPKSCDKTHTCPP |
| | | CPAPELLGGPSVFLFPPKPKDTLMISRTPEVTCVVVDVSHEDP |
| | | EVKFNWYVDGVEVHNAKTKPREEQYNSTYRVVSVLTVLHQ |
| | | DWLNGKEYKCKVSNKALPAPIEKTISKAKGQPREPQVYTLPP |
| | | SREEMTKNQVSLTCLVKGFYPSDIAVEWESNGQPENNYKTTP |
| | | PVLDSDGSFFLYSKLTVDKSRWQQGNVFSCSVMHEALHNHY |
| | | TQKSLSLSPGK |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 11 | VL | DIQMTQSPSSLSASVGDRVTITCKASQNVGINVVWHQQKPGK |
| | | VPKALIYSSSHRYSGVPSRFSGSGSGTDFTLTISSLQPEDVATY |
| | | FCQQFKSYPLTFGQGTKLEIK |
| SEQ ID NO: 14 | DNA VL | GACATCCAGATGACCCAGTCTCCATCCTCTCTGTCCGCCTC |
| | | TGTGGGCGACAGAGTGACCATCACATGCAAGGCCTCTCAG |
| | | AACGTGGGCATCAACGTCGTGTGGCACCAGCAGAAACCTG |
| | | GCAAGGTGCCCAAGGCTCTGATCTACTCCTCCAGCCACAG |
| | | ATACTCCGGCGTGCCCTCTAGATTCTCCGGCTCTGGCTCTG |
| | | GCACCGACTTTACCCTGACAATCTCCAGCCTGCAGCCTGA |
| | | GGACGTGGCCACCTACTTTTGCCAGCAGTTCAAGAGCTAC |
| | | CCTCTGACCTTTGGCCAGGGCACCAAGCTGGAAATCAAG |
| SEQ ID NO: | Light chain | METDTLLLWVLLLWVPGSTGDIQMTQSPSSLSASVGDRVTIT |
| 3279 | | CKASQNVGINVVWHQQKPGKVPKALIYSSSHRYSGVPSRFSG |
| | | SGSGTDFTLTISSLQPEDVATYFCQQFKSYPLTFGQGTKLEIKR |
| | | TVAAPSVFIFPPSDEQLKSGTASVVCLLNNFYPREAKVQWKV |
| | | DNALQSGNSQESVTEQDSKDSTYSLSSTLTLSKADYEKHKVY |
| | | ACEVTHQGLSSPVTKSFNRGEC |

A-H.3 antibody

| | | |
|---|---|---|
| SEQ ID NO: 80 | VH + VL | QVQLVQSGAEVKKPGSSVKVSCKASGTDFKLTYIHWVRQAP |
| | | GQGLEWMGRVSPGSQNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVQDRVTITCKAS |
| | | QNVEDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: 81 | VL | DIQMTQSPSFLSASVGDRVTITCKASQNVEDRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 82 | VH | QVQLVQSGAEVKKPGSSVKVSCKASGTDFKLTYIHWVRQAP |
| | | GQGLEWMGRVSPGSGNTKYNEKFKGRVTITADTSTSTAYME |
| | | LSSLRSEDTAVYYCAGSYYSYDVLDYWGQGTTVTVSS |

A-H.4

| | | |
|---|---|---|
| SEQ ID NO: 83 | VH + VL | QVQLVQSGAEVKKPGSSVKVSCKASGTDFDKIYIHWVRQAP |
| | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: 84 | VL | DIQMTQSPSFLSASVGDRVTITCKASQNVEDRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 85 | VH | QVQLVQSGAEVKKPGSSVKVSCKASGTDFDKIYIHWVRQAP |
| | | GQGLEWMGRISAGSGNVKYNEKFKGRVTITADTSTSTAYME |
| | | LSSLRSEDTAVYYCAGSYYSYDVLDYWGQGTTVTVSS |

A-H.5

| | | |
|---|---|---|
| SEQ ID NO: 86 | VH + VL | QVQLVQSGAEVKKPGSSVKVSCKASGHDFRDFYIHWVRQAP |
| | | GQGLEWMGRVYPGSGSYRYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: 87 | VL | DIQMTQSPSFLSASVGDRVTITCKASQNVDDRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 88 | VH | QVQLVQSGAEVKKPGSSVKVSCKASGHDFRDFYIHWVRQAP |
| | | GQGLEWMQRVYPGSGSYRYNEKFKGRVTITADTSTSTAYME |
| | | LSSLRSEDTAVYYCAGSYYSYDVLDYWGQGTTVTVSS |

A-H.6

| | | |
|---|---|---|
| SEQ ID NO: 89 | VH + VL | QVQLVQSGAEVKKPGSSVKVSCKASGHDFKLTYIHWVRQAP |
| | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: 90 | VL | DIQMTQSPSFLSASVGDRVTITCKASQNVDNRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 91 | VH | QVQLVQSGAEVKKPGSSVKVSCKASGHDFKLTYIHWVRQAP |
| | | GQGLEWMGRISAGSGNVKYNEKFKGRVTITADTSTSTAYME |
| | | LSSLRSEDTAVYYCAGSYYSYDVLDYWGQGTTVTVSS |

A-H.7

| | | |
|---|---|---|
| SEQ ID NO: 92 | VH + VL | QVQLVQSGAEVKKPGSSVKVSCKASGTDFKLTYIHWVRQAP |
| | | GQGLEWMGRIFPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVENKVAWHQQKPGKAPKALIYSSSHRYKGVPSRFSGSGS |
| | | GTEFTLTISSLQPEDFATYFCQQFKSYPLTFGQGTKLEIK |
| SEQ ID NO: 93 | VL | DIQMTQSPSFLSASVGDRVTITCKASQNVENKVAWHQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 94 | VH | QVQLVQSGAEVKKPGSSVKVSCKASGTDFKLTYIHWVRQAP |
| | | GQGLEWMGRIFPGSGNVKYNEKFKGRVTITADTSTSTAYME |
| | | LSSLRSEDTAVYYCAGSYYSYDVLDYWGQGTTVTVSS |

A-H.8

| | | |
|---|---|---|
| SEQ ID NO: 95 | VH + VL | QVQLVQSGAEVKKPGSSVKVSCKASGTDFDKIYIHWVRQAP |
| | | GQGLEWMGRIFAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: 96 | VL | DIQMTQSPSFLSASVGDRVTITCKASQNVDDRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 97 | VH | QVQLVQSGAEVKKPGSSVKVSCKASGTDFDKIYIHWVRQAP |
| | | GQGLEWMGRIFAGSGNTKYNEKFKGRVTITADTSTSTAYME |
| | | LSSLRSEDTAVYYCAGSYYSYDVLDYWGQGTTVTVSS |

A-H.9

| | | |
|---|---|---|
| SEQ ID NO: 98 | VH + VL | QVQLVQSGAEVKKPGSSVKVSCKASGHDFDKFYIHWVRQAP |
| | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGNRVAWYQQKPGKAPKALIYSSSHRYSGVPSRFSGSGS |
| | | GTEFTLTISSLQPEDFATYFCQQFKSYPLTFGQGTKLEIK |
| SEQ ID NO: 99 | VL | DIQMTQSPSFLSASVGDRVTITCKASQNVGNRVAWYQQKPG |
| | | KAPKALIYSSSHRYSGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKFYIHWVRQAP |
| 100 | | GQGLEWMGRVSAGSGNVKYNEKFKGRVTITADTSTSTAYME |
| | | LSSLRSEDTAVYYCAGSYYSYDVLDYWGQGTTVTVSS |

A-H.10

| | | |
|---|---|---|
| SEQ ID NO: | VH + VL | QVQLVQSGAEVKKPGSSVKVSCKASGHDFDKFYIHWVRQAP |
| 101 | | GQGLEWMGRIFAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVAWYQQKPGKAPKALIYSSSHRYKGVPSRFSGSGS |
| | | GTEFTLTISSLQPEDFATYFCQQFKSYPLTFGQGTKLEIKs |
| SEQ ID NO: | VL | DIQMTQSPSFLSASVGDRVTITCKASQNVGDRVAWYQQKPG |
| 102 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKFYIHWVRQAP |
| 103 | | GQGLEWMGRIFAGSGNVKYNEKFKGRVTITADTSTSTAYME |
| | | LSSLRSEDTAVYYCAGSYYSYDVLDYWGQGTTVTVSS |

A-H.11

| | | |
|---|---|---|
| SEQ ID NO: | VH + VL | QVQLVQSGAEVKKPGSSVKVSCKASGTDFKLTYIHWVRQAP |
| 104 | | GQGLEWMGRVSPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO | VL | DIQMTQSPSFLSASVGDRVTITCKASQNVGDRVAWYQQKPG |
| 105 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 106 | | GQGLEWMGRVSPGSGNVKYNEKFKGRVTITADTSTSTAYME |
| | | LSSLRSEDTAVYYCAGSYYSYDVLDYWGQGTTVTVSS |

A-H.12

| | | |
|---|---|---|
| SEQ ID NO: | VH + VL | QVQLVQSGAEVKKPGSSVKVSCKASGTDFDKIYIHWVRQAP |
| 107 | | GQGLEWMGRVSAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGNRVAWYQQKPQKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGNRVAWYQQKPG |
| 108 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 109 | | GQGLEWMGRVSAGSGNTKYNEKFKGRVTITADTSTSTAYME |
| | | LSSLRSEDTAVYYCAGSYYSYDVLDYWGQGTTVTVSS |

A-H.13, also referred to as A-H.69

| | | |
|---|---|---|
| SEQ ID NO: | VH + VL | QVQLVQSGAEVKKPGSSVKVSCKASGTDFKLTYIHWVRQAP |
| 110 | | GQGLEWMGRIFPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDNRVAWYQQKPG |
| 111 | | KAPKALIYSSSHRYKGVPSRFSGSGSGTEFTLTISSLQPED |
| | | FATYFCQQFKSYPLTFGQGTKLEIK |
| SEQ ID NO: | VH | QVQLVQSGAEVKKPGSSVKVSCKASGTDFKLTYIHWVRQAP |
| 112 | | GQGLEWMGRIFPGSGNVKYNEKFKGRVTITADTSTSTAYME |
| | | LSSLRSEDTAVYYCAGSYYSYDVLDYWGQGTTVTVSS |

A-H.14

| | | |
|---|---|---|
| SEQ ID NO: | VH + VL | QVQLVQSGAEVKKPGSSVKVSCKASGTDFDKIYIHWVRQAP |
| 113 | | GQGLEWMGRISAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 114 | | KAPKALIYSSSHRYKGVPSRFSGSGSGTEFTLTISSLQPED |
| | | FATYFCQQFKSYPLTFGQGTKLEIK |
| SEQ ID NO: | VH | QVQLVQSGAEVKKPGSSVKVSCKASGTDFDKIYIHWVRQAP |
| 115 | | GQGLEWMGRISAGSGNTKYNEKFKGRVTITADTSTSTAYME |
| | | LSSLRSEDTAVYYCAGSYYSYDVLDYWGQGTTVTVSS |

A-H.15

| | | |
|---|---|---|
| SEQ ID NO: | VH + VL | QVQLVQSGAEVKKPGSSVKVSCKASGTDFRLTYIHWVRQAP |
| 116 | | GQGLEWMGRVSPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNKVAWHQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDNKVAWHQQKPG |
| 117 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFRLTYIHWVRQAP |
| 118 | | GQGLEWMGRVSPGSGNTKYNEKFKGRVTITADTSTSTAYME |
| | | LSSLRSEDTAVYYCAGSYYSYDVLDYWGQGTTVTVSS |

A-H.16

| | | |
|---|---|---|
| SEQ ID NO: | VH + VL | QVQLVQSGAEVKKPGSSVKVSCKASGGTFRLTYIHWVRQAP |
| 119 | | GQGLEWMGRVYPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 120 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGGTFRLTYIHWVRQAP |
| 121 | | GQGLEWMGRVYPGSGNTKYNEKFKGRVTITADTSTSTAYME |
| | | LSSLRSEDTAVYYCAGSYYSYDVLDYWGQGTTVTVSS |

A-H.17

| | | |
|---|---|---|
| SEQ ID NO: | VH + VL | QVQLVQSGAEVKKPGSSVKVSCKASGTDFRLTYIHWVRQAP |
| 122 | | GQGLEWMGRIFPGSGNTKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVDDRVAWYQQKPGKAPKALIYSSSHRYKGVPSRFSGSGSG |
| | | TEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 123 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFRLTYIHWVRQAP |
| 124 | | GQGLEWMGRIFPGSGNTKYNEKFKGRVTITADTSTSTAYMEL |
| | | SSLRSEDTAVYYCAGSYYSYDVLDYWGQGTTVTVSS |

A-H.18

| | | |
|---|---|---|
| SEQ ID NO: | VH + VL | QVQLVQSGAEVKKPGSSVKVSCKASGTDFKLTYIHWVRQAP |
| 125 | | GQGLEWMGRIFPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVEDRVAWYQQKPG |
| 126 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 127 | | GQGLEWMGRIFPGSGNVKYNEKFKGRVTITADTSTSTAYME |
| | | LSSLRSEDTAVYYCAGSYYSYDVLDYWGQGTTVTVSS |

A-H.19

| | | |
|---|---|---|
| SEQ ID NO: | VH + VL | QVQLVQSGAEVKKPGSSVKVSCKASGGTFRLTYIHWVRQAP |
| 128 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 129 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGGTFRLTYIHWVRQAP |
| 130 | | GQGLEWMGRISAGSGNVKYNEKFKGRVTITADTSTSTAYME |
| | | LSSLRSEDTAVYYCAGSYYSYDVLDYWGQGTTVTVSS |

A-H.20

| | | |
|---|---|---|
| SEQ ID NO: | VH + VL | QVQLVQSGAEVKKPGSSVKVSCKASGGTFDKTYIHWVRQAP |
| 131 | | GQGLEWMGRISAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO | VL | DIQMTQSPSFLSASVGDRVTITCKASQNVDDRVAWYQQKPG |
| 132 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGGTFDKTYIHWVRQAP |
| 133 | | GQGLEWMGRISAGSGNTKYNEKFKGRVTITADTSTSTAYME |
| | | LSSLRSEDTAVYYCAGSYYSYDVLDYWGQGTTVTVSS |

A-H.21

| | | |
|---|---|---|
| SEQ ID NO: | VH + VL | QVQLVQSGAEVKKPGSSVKVSCKASGHDFDKFYIHWVRQAP |
| 134 | | GQGLEWMGRISAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPQKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 135 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKFYIHWVRQAP |
| 136 | | GQGLEWMGRISAGSGNTKYNEKFKGRVTITADTSTSTAYME |
| | | LSSLRSEDTAVYYCAGSYYSYDVLDYWGQGTTVTVSS |

A-H.22

| | | |
|---|---|---|
| SEQ ID NO: | VH + VL | QVQLVQSGAEVKKPGSSVKVSCKASGTDFKLTYIHWVRQAP |
| 137 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNKVAWHQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDNKVAWHQQKPG |
| 138 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 139 | | GQGLEWMGRISAGSGNVKYNEKFKGRVTITADTSTSTAYME |
| | | LSSLRSEDTAVYYCAGSYYSYDVLDYWGQGTTVTVSS |

A-H.23

| | | |
|---|---|---|
| SEQ ID NO: | VH + VL | QVQLVQSGAEVKKPGSSVKVSCKASGHDFRLTYIHWVRQAP |
| 140 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVADRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVADRVAWYQQKPG |
| 141 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFRLTYIHWVRQAP |
| 142 | | GQGLEWMGRISAGSGNVKYNEKFKGRVTITADTSTSTAYME |
| | | LSSLRSEDTAVYYCAGSYYSYDVLDYWGQGTTVTVSS |

A-H.24

| | | |
|---|---|---|
| SEQ ID NO: | VH + VL | QVQLVQSGAEVKKPGSSVKVSCKASGHDFHLWYIHWVRQA |
| 143 | | PGQGLEWMGRVSAGSGNVKYNEKFKGRVTITADTSTSTAYM |
| | | ELSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVDNKVAWHQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVDNKVAWHQQKPG |
| 144 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFHLWYIHWVRQA |
| 145 | | PGQGLEWMGRVSAGSGNVKYNEKFKGRVTITADTSTSTAYM |
| | | ELSSLRSEDTAVYYCAGSYYSYDVLDYWGQGTTVTVSS |

A-H.25

| | | |
|---|---|---|
| SEQ ID NO: | VH + VL | QVQLVQSGAEVKKPGSSVKVSCKASGHDFHLWYIHWVRQA |
| 146 | | PGQGLEWMGRVFAGSGNTKYNEKFKGRVTITADTSTSTAYM |
| | | ELSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVEDKVAWYQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVEDKVAWYQQKPG |
| 147 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFHLWYIHWVRQA |
| 148 | | PGQGLEWMGRVFAGSGNTKYNEKFKGRVTITADTSTSTAYM |
| | | ELSSLRSEDTAVYYCAGSYYSYDVLDYWGQGTTVTVSS |

A-H.26

| | | |
|---|---|---|
| SEQ ID NO: | VH + VL | QVQLVQSGAEVKKPGSSVKVSCKASGTDFKLTYIHWVRQAP |
| 149 | | GQGLEWMGRIFPGSGNTKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVDDRVAWYQQKPGKAPKALIYSSSHRYKGVPSRFSGSGSG |
| | | TEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 150 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 151 | | GQGLEWMGRIFPGSGNTKYNEKFKGRVTITADTSTSTAYMEL |
| | | SSLRSEDTAVYYCAGSYYSYDVLDYWGQGTTVTVSS |

A-H.27

| | | |
|---|---|---|
| SEQ ID NO: | VH + VL | QVQLVQSGAEVKKPGSSVKVSCKASGTDFKLTYIHWVRQAP |
| 153 | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGNRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGNRVAWYQQKPG |
| 154 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 155 | | GQGLEWMGRVSAGSGNVKYNEKFKGRVTITADTSTSTAYME |
| | | LSSLRSEDTAVYYCAGSYYSYDVLDYWGQGTTVTVSS |

A-H.28

| | | |
|---|---|---|
| SEQ ID NO: | VH + VL | QVQLVQSGAEVKKPGSSVKVSCKASGTDFKLTYIHWVRQAP |
| 156 | | GQGLEWMGRISPGSGNTKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVGDRVAWYQQKPGKAPKALIYSSSHRYKGVPSRFSGSGSG |
| | | TEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 157 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 158 | | GQGLEWMGRISPGSGNTKYNEKFKGRVTITADTSTSTAYMEL |
| | | SSLRSEDTAVYYCAGSYYSYDVLDYWGQGTTVTVSS |

A-H.29

| | | |
|---|---|---|
| SEQ ID NO: | VH + VL | QVQLVQSGAEVKKPGSSVKVSCKASGHDFHLWYIHWVRQA |
| 159 | | PGQGLEWMGRISPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVAWHQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGDRVAWHQQKPG |
| 160 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFHLWYIHWVRQA |
| 161 | | PGQGLEWMGRISPGSGNVKYNEKFKGRVTITADTSTSTAYME |
| | | LSSLRSEDTAVYYCAGSYYSYDVLDYWGQGTTVTVSS |

A-H.31

| | | |
|---|---|---|
| SEQ ID NO: | VH + VL | QVQLVQSGAEVKKPGSSVKVSCKASGHDFKLTYIHWVRQAP |
| 162 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 163 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFKLTYIHWVRQAP |
| 164 | | GQGLEWMGRISAGSGNVKYNEKFKGRVTITADTSTSTAYME |
| | | LSSLRSEDTAVYYCAGSYYSYDVLDYWGQGTTVTVSS |

A-H.31

| | | |
|---|---|---|
| SEQ ID NO: | VH + VL | QVQLVQSGAEVKKPGSSVKVSCKASGTDFHLWYIHWVRQAP |
| 165 | | GQGLEWMGRVFAGSGSYRYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 166 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFHLWYIHWVRQAP |
| 167 | | GQGLEWMGRVFAGSGSYRYNEKFKGRVTITADTSTSTAYME |
| | | LSSLRSEDTAVYYCAGSYYSYDVLDYWGQGTTVTVSS |

A-H.32

| | | |
|---|---|---|
| SEQ ID NO: | VH + VL | QVQLVQSGAEVKKPGSSVKVSCKASGTDFDKIYIHWVRQAP |
| 168 | | GQGLEWMGRISAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVADRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVADRVAWYQQKPG |
| 169 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 170 | | GQGLEWMGRISAGSGNTKYNEKFKGRVTITADTSTSTAYME |
| | | LSSLRSEDTAVYYCAGSYYSYDVLDYWGQGTTVTVSS |

A-H.33

| | | |
|---|---|---|
| SEQ ID NO: | VH + VL | QVQLVQSGAEVKKPGSSVKVSCKASGTDFKLTYIHWVRQAP |
| 171 | | GQGLEWMGRISAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVEDRVAWYQQKPG |
| 172 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 173 | | GQGLEWMGRISAGSGNTKYNEKFKGRVTITADTSTSTAYME |
| | | LSSLRSEDTAVYYCAGSYYSYDVLDYWGQGTTVTVSS |

A-H.34

| | | |
|---|---|---|
| SEQ ID NO: | VH + VL | QVQLVQSGAEVKKPGSSVKVSCKASGTDFRLTYIHWVRQAP |
| 174 | | GQGLEWMGRISPGSGNTKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVGNRVAWYQQKPGKAPKALIYSSSHRYKGVPSRFSGSGSG |
| | | TEFTLTISSLQPEDFATYFCQQFKSYPLTFGQGTKLEIK |
| SEQ ID NO: | VL | DIQMTQSPSFLSASVGDRVTITCKASQNVGNRVAWYQQKPG |
| 175 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFRLTYIHWVRQAP |
| 176 | | GQGLEWMGRISPGSGNTKYNEKFKGRVTITADTSTSTAYMEL |
| | | SSLRSEDTAVYYCAGSYYSYDVLDYWGQGTTVTVSS |

A-H.35

| | | |
|---|---|---|
| SEQ ID NO: | VH + VL | QVQLVQSGAEVKKPGSSVKVSCKASGHDFDKTYIHWVRQAP |
| 177 | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVEDRVAWYQQKPG |
| 178 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKTYIHWVRQAP |
| 179 | | GQGLEWMGRVSAGSGNVKYNEKFKGRVTITADTSTSTAYME |
| | | LSSLRSEDTAVYYCAGSYYSYDVLDYWGQGTTVTVSS |

A-H.36

| | | |
|---|---|---|
| SEQ ID NO: | VH + VL | QVQLVQSGAEVKKPGSSVKVSCKASGHDFKLTYIHWVRQAP |
| 180 | | GQGLEWMGRVSPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWHQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVEDRVAWHQQKPG |
| 181 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFKLTYIHWVRQAP |
| 182 | | GQGLEWMGRVSPGSGNTKYNEKFKGRVTITADTSTSTAYME |
| | | LSSLRSEDTAVYYCAGSYYSYDVLDYWGQGTTVTVSS |

A-H.37

| | | |
|---|---|---|
| SEQ ID NO: | VH + VL | QVQLVQSGAEVKKPGSSVKVSCKASGHDEDKTYIHWVRQAP |
| 183 | | GQGLEWMGRIYPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVADRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVADRVAWYQQKPG |
| 184 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKTYIHWVRQAP |
| 185 | | GQGLEWMGRIYPGSGNVKYNEKFKGRVTITADTSTSTAYME |
| | | LSSLRSEDTAVYYCAGSYYSYDVLDYWGQGTTVTVSS |

A-H.38

| | | |
|---|---|---|
| SEQ ID NO: | VH + VL | QVQLVQSGAEVKKPGSSVKVSCKASGTDFDKTYIHWVRQAP |
| 186 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 187 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKTYIHWVRQAP |
| 188 | | GQGLEWMGRISAGSGNVKYNEKFKGRVTITADTSTSTAYME |
| | | LSSLRSEDTAVYYCAGSYYSYDVLDYWGQGTTVTVSS |

A-H.39

| | | |
|---|---|---|
| SEQ ID NO: | VH + VL | QVQLVQSGAEVKKPGSSVKVSCKASGTDFDKIYIHWVRQAP |
| 189 | | GQGLEWMGRISAGSGNIKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVDDRVAWYQQKPGKAPKALIYSSSHRYKGVPSRFSGSGSG |
| | | TEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 190 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 191 | | GQGLEWMGRISAGSGNIKYNEKFKGRVTITADTSTSTAYMEL |
| | | SSLRSEDTAVYYCAGSYYSYDVLDYWGQGTTVTVSS |

A-H.40

| | | |
|---|---|---|
| SEQ ID NO: | VH + VL | QVQLVQSGAEVKKPGSSVKVSCKASGTDFDKIYIHWVRQAP |
| 192 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 193 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 194 | | GQGLEWMGRISAGSGNVKYNEKFKGRVTITADTSTSTAYME |
| | | LSSLRSEDTAVYYCAGSYYSYDVLDYWGQGTTVTVSS |

A-H.41

| | | |
|---|---|---|
| SEQ ID NO: | VH + VL | QVQLVQSGAEVKKPGSSVKVSCKASGGTFKLTYIHWVRQAP |
| 195 | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 196 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGGTFKLTYIHWVRQAP |
| 197 | | GQGLEWMGRVSAGSGNVKYNEKFKGRVTITADTSTSTAYME |
| | | LSSLRSEDTAVYYCAGSYYSYDVLDYWGQGTTVTVSS |

A-H.42

| | | |
|---|---|---|
| SEQ ID NO: | VH + VL | QVQLVQSGAEVKKPGSSVKVSCKASGTDFKLTYIHWVRQAP |
| 198 | | GQGLEWMGRISPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNRVAWHQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDNRVAWHQQKPG |
| 199 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 200 | | GQGLEWMGRISPGSGNVKYNEKFKGRVTITADTSTSTAYME |
| | | LSSLRSEDTAVYYCAGSYYSYDVLDYWGQGTTVTVSS |

A-H.43

| | | |
|---|---|---|
| SEQ ID NO: | VH + VL | QVQLVQSGAEVKKPQSSVKVSCKASGHDFDKFYIHWVRQAP |
| 201 | | GQGLEWMGRVSAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVQDRVTITCKAS |
| | | QNVDNRVAWYQQKPQKAPKALIYSSSHRYKGVPSRFSGSQS |
| | | GTEFTLTISSLQPEDFATYFCQQFKSYPLTFGQGTKLEIK |
| SEQ ID NO: | VL | DIQMTQSPSFLSASVGDRVTITCKASQNVDNRVAWYQQKPG |
| 202 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKFYIHWVRQAP |
| 203 | | GQGLEWMGRVSAGSGNTKYNEKFKGRVTITADTSTSTAYME |
| | | LSSLRSEDTAVYYCAGSYYSYDVLDYWGQGTTVTVSS |

A-H.44

| | | |
|---|---|---|
| SEQ ID NO: | VH + VL | QVQLVQSGAEVKKPGSSVKVSCKASGTDFDKFYIHWVRQAP |
| 204 | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVVWYQQKPGKAPKALIYSSSHRYKGVPSRFSGSGS |
| | | GTEFTLTISSLQPEDFATYFCQQFKSYPLTFGQGTKLEIK |
| SEQ ID NO: | VH | QVQLVQSGAEVKKPGSSVKVSCKASGTDFDKFYIHWVRQAP |
| 205 | | GQGLEWMGRVSAGSGNVKYNEKFKGRVTITADTSTSTAYME |
| | | LSSLRSEDTAVYYCAGSYYSYDVLDYWGQGTTVTVSS |

A-H.45

| | | |
|---|---|---|
| SEQ ID NO: | VH + VL | QVQLVQSGAEVKKPGSSVKVSCKASGYSFTTYYIHWVRQAP |
| 206 | | GQGLEWMQWFSAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 207 | | GQGLEWMGWFSAGSGNTKYNEKFKGRVTITADTSTSTAYME |
| | | LSSLRSEDTAVYYCAVSYYSYDVLDYWGQGTTVTVSS |

A-H.46

| | | |
|---|---|---|
| SEQ ID NO: | VH + VL | QVQLVQSGAEVKKPGSSVKVSCKASGYSFTTYYIHWVRQAP |
| 208 | | GQGLEWMGWFSAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 209 | | GQGLEWMGWFSAGSGNTKYNEKFKGRVTITADTSTSTAYME |
| | | LSSLRSEDTAVYYCAGSYYSYDVLDYWGQGTTVTVSS |

A-H.47

| | | |
|---|---|---|
| SEQ ID NO: | VH + VL | QVQLVQSGAEVKKPGSSVKVSCKASGYSFTTYYIHWVRQAP |
| 210 | | GQGLEWMGWFFPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 211 | | GQGLEWMGWFFPGSGNTKYNEKFKGRVTITADTSTSTAYME |
| | | LSSLRSEDTAVYYCAGSYYSYDVLDYWGQGTTVTVSS |

A-H.48

| | | |
|---|---|---|
| SEQ ID NO: | VH + VL | QVQLVQSGAEVKKPQSSVKVSCKASGYSFTTYYIHWVRQAP |
| 212 | | GQGLEWMGWFSPGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVQDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 213 | | GQGLEWMGWFSPGSGNTKYNEKFKGRVTITADTSTSTAYME |
| | | LSSLRSEDTAVYYCAVSYYSYDVLDYWGQGTTVTVSS |

A-H.49

| | | |
|---|---|---|
| SEQ ID NO: | VH + VL | QVQLVQSGAEVKKPGSSVKVSCKASGYSFTTYYIHWVRQAP |
| 214 | | GQGLEWMGWFSPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 215 | | GQGLEWMGWFSPGSGNTKYNEKFKGRVTITADTSTSTAYME |
| | | LSSLRSEDTAVYYCAGSYYSYDVLDYWGQGTTVTVSS |

A-H.50

| | | |
|---|---|---|
| SEQ ID NO: | VH + VL | QVQLVQSGAEVKKPGSSVKVSCKASGYSFTTYYIHWVRQAP |
| 216 | | GQGLEWMGRIFPGSGNIKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVGINVVWHQQKPGKAPKALIYSSSHRYSGVPSRFSGSGSGT |
| | | EFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 217 | | GQGLEWMGRIFPGSGNIKYNEKFKGRVTITADTSTSTAYMEL |
| | | SSLRSEDTAVYYCAGSYYSYDVLDYWGQGTTVTVSS |

A-H.51

| | | |
|---|---|---|
| SEQ ID NO: | VH + VL | QVQLVQSGAEVKKPGSSVKVSCKASGYSFTTYYIHWVRQAP |
| 218 | | GQGLEWMGWFFPGSGNIKYNEKFKGRVTITADTSTSTAYME |
| | | LSSLRSEDTAVYYCAGSIYSAGVLDYWGQGTTVTVSSGGGGS |
| | | GGGGSGGGGSGGGGSDIQMTQSPSFLSASVGDRVTITCKASQ |
| | | NVGINVVWHQQKPGKAPKALIYSSSHRYSGVPSRFSGSGSGT |
| | | EFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 219 | | GQGLEWMGWFFPGSGNIKYNEKFKGRVTITADTSTSTAYME |
| | | LSSLRSEDTAVYYCAGSIYSAGVLDYWGQGTTVTVSS |

A-H.52

| | | |
|---|---|---|
| SEQ ID NO: | VH + VL | QVQLVQSGAEVKKPGSSVKVSCKASGYSFTLGYIHWVRQAP |
| 220 | | GQGLEWMGWFFPGSGNIKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTLGYIHWVRQAP |
| 221 | | GQGLEWMGWFFPGSGNIKYNEKFKGRVTITADTSTSTAYME |
| | | LSSLRSEDTAVYYCAGSYYSYDVLDYWGQGTTVTVSS |

A-H.53

| | | |
|---|---|---|
| SEQ ID NO: | VH + VL | QVQLVQSGAEVKKPGSSVKVSCKASGYSFRLTYIHWVRQAP |
| 222 | | GQGLEWMGWFFPGSGNIKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFRLTYIHWVRQAP |
| 223 | | GQGLEWMGWFFPGSGNIKYNEKFKGRVTITADTSTSTAYME |
| | | LSSLRSEDTAVYYCAGSYYSYDVLDYWGQGTTVTVSS |

A-H.54

| | | |
|---|---|---|
| SEQ ID NO: | VH + VL | QVQLVQSGAEVKKPGSSVKVSCKASGYSFHNWYIHWVRQA |
| 224 | | PGQGLEWMGWFFPGSGNIKYNEKFKGRVTITADTSTSTAYM |
| | | ELSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVGINVVWHQQKPGKAPKALIYSSSHRYSGVPSRFSGSGS |
| | | GTEFTLTISSLQPEDFATYFCQQFKSYPLTFGQGTKLEIK |
| SEQ ID NO: | VH | QVQLVQSGAEVKKPGSSVKVSCKASGYSFHNWYIHWVRQA |
| 225 | | PGQGLEWMGWFFPGSGNIKYNEKFKGRVTITADTSTSTAYM |
| | | ELSSLRSEDTAVYYCAGSYYSYDVLDYWGQGTTVTVSS |

A-H.55 antibody

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 45 | HC CDR1 (Kabat) | TYYIH |
| SEQ ID NO: 46 | HC CDR2 (Kabat) | WFFPGSGNIKYNEKFKG |
| SEQ ID NO: 47 | HC CDR3 (Kabat) | SYYSYDVLDY |
| SEQ ID NO: 48 | HC CDR1 (Chothia) | GYSFTTY |
| SEQ ID NO: 49 | HC CDR2 (Chothia) | FPGSGN |
| SEQ ID NO: 50 | HC CDR3 (Chothia) | SYYSYDVLDY |
| SEQ ID NO: | VH | QVQLVQSGAEVKKPGSSVKVSCKASGYSFTTYYIHWVRQAP |
| 1100 | | GQGLEWMGWFFPGSGNIKYNEKFKGRVTITADTSTSTAYME |
| | | LSSLRSEDTAVYYCAGSYYSYDVLDYWGQGTTVTVSS |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 51 | LC CDR1 (Kabat) | KASQNVGINVV |
| SEQ ID NO: 52 | LC CDR2 (Kabat) | SSSHRYS |
| SEQ ID NO: 53 | LC CDR3 (Kabat) | QQFKSYPLT |
| SEQ ID NO: 54 | LC CDR1 (Chothia) | KASQNVGINVV |
| SEQ ID NO: 55 | LC CDR2 (chothia) | SSSHRYS |
| SEQ ID NO: 56 | LC CDR3 (chothia) | QQFKSYPLT |
| SEQ ID NO: | VL | QSVLTQPPSVSEAPRQRVTISCKASQNVGINVVWHQQLPGKA |
| 1101 | | PKALIYSSSHRYSGVSDRFSGSGSGTSFSLAISGLQSEDEADYF |
| | | CQQFKSYPLTFGTGTKVTVL |

A-H.56

| | | |
|---|---|---|
| SEQ ID NO: | VH + VL (ScFv) | QVQLVQSGAEVKKPGSSVKVSCKASGHDEDKFYIHWVRQAP |
| 1309 | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGNRVAWYQQKPGKAPKALIYSSSHRYKGVPSRFSGSGS |
| | | GTEFTLTISSLQPEDFATYFCQQFKSYPLTFGQGTKLEIK |

A-H.57

| | | |
|---|---|---|
| SEQ ID NO: | VH + VL (ScFv) | QVQLVQSGAEVKKPGSSVKVSCKASGHDFRLTYIHWVRQAP |
| 1326 | | GQGLEWMGRVSAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGDRVVWHQQKPGKAPKALIYSSSHRYKGVPSRFSGSGS |
| | | GTEFTLTISSLQPEDFATYFCQQFKSYPLTFGQGTKLEIK |

A-H.58

| | | |
|---|---|---|
| SEQ ID NO: | VH + VL (ScFv) | QVQLVQSGAEVKKPGSSVKVSCKASGHDFRLTYIHWVRQAP |
| 1327 | | GQGLEWMGRVSAGSGNVKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGNRVVWHQQKPGKAPKALIYSSSHRYKGVPSRFSGSGS |
| | | GTEFTLTISSLQPEDFATYFCQQFKSYPLTFGQGTKLEIK |

A-H.59

| | | |
|---|---|---|
| SEQ ID NO: | VH + VL (ScFv) | QVQLVQSGAEVKKPGSSVKVSCKASGHDFRLTYIHWVRQAP |
| 1328 | | GQGLEWMGRIYAGSGNVKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVADRVVWHQQKPGKAPKALIYSSSHRYKGVPSRFSGSGS |
| | | GTEFTLTISSLQPEDFATYFCQQFKSYPLTFGQGTKLEIK |

A-H.60

| | | |
|---|---|---|
| SEQ ID NO: | VH + VL (ScFv) | QVQLVQSGAEVKKPGSSVKVSCKASGHDFKLTYIHWVRQAP |
| 1329 | | GQGLEWMGRVSAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGDRVAWHQQKPGKAPKALIYSSSHRYKGVPSRFSGSGS |
| | | GTEFTLTISSLQPEDFATYFCQQFKSYPLTFGQGTKLEIK |

A-H.61

| | | |
|---|---|---|
| SEQ ID NO: | VH + VL (ScFv) | QVQLVQSGAEVKKPGSSVKVSCKASGHDFKLTYIHWVRQAP |
| 1330 | | GQGLEWMGRVSAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVDNRVAWHQQKPGKAPKALIYSSSHRYKGVPSRFSGSGS |
| | | GTEFTLTISSLQPEDFATYFCQQFKSYPLTFGQGTKLEIK |

A-H.62

| | | |
|---|---|---|
| SEQ ID NO: | VH + VL (ScFv) | QVQLVQSGAEVKKPGSSVKVSCKASGHDFRLTYIHWVRQAP |
| 1331 | | GQGLEWMGRVSAGSGNVKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVADRVVWHQQKPGKAPKALIYSSSHRYKGVPSRFSGSGS |
| | | GTEFTLTISSLQPEDFATYFCQQFKSYPLTFGQGTKLEIK |

A-H.63

| | | |
|---|---|---|
| SEQ ID NO: | VH + VL (ScFv) | QVQLVQSGAEVKKPGSSVKVSCKASGHDFRLTYIHWVRQAP |
| 1332 | | GQGLEWMGRVYAGSGNTKYNEKFKGRVTITADTSTSTAYM |
| | | ELSSLRSEDTAVYYCAVSYYSYDVLDYWGQGTTVTVSSGGG |
| | | GSGGGGSGGGGSGGGGSDIQMTQSPSFLSASVGDRVTITCKA |
| | | SQNVEDRVVWHQQKPGKAPKALIYSSSHRYKGVPSRFSGSGS |
| | | GTEFTLTISSLQPEDFATYFCQQFKSYPLTFGQGTKLEIK |

A-H.64

| | | |
|---|---|---|
| SEQ ID NO: | VH + VL (ScFv) | QVQLVQSGAEVKKPGSSVKVSCKASGHDFKLTYIHWVRQAP |
| 1333 | | GQGLEWMGRVSAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVADRVVWHQQKPGKAPKALIYSSSHRYKGVPSRFSGSGS |
| | | GTEFTLTISSLQPEDFATYFCQQFKSYPLTFGQGTKLEIK |

A-H.65

| | | |
|---|---|---|
| SEQ ID NO: | VH + VL (ScFv) | QVQLVQSGAEVKKPGSSVKVSCKASGHDFKLTYIHWVRQAP |
| 1334 | | GQGLEWMGRISAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGDRVVWHQQKPGKAPKALIYSSSHRYKGVPSRFSGSGS |
| | | GTEFTLTISSLQPEDFATYFCQQFKSYPLTFGQGTKLEIK |

A-H.66

| | | |
|---|---|---|
| SEQ ID NO: | VH + VL (ScFv) | QVQLVQSGAEVKKPGSSVKVSCKASGHDFKLTYIHWVRQAP |
| 1335 | | GQGLEWMGRIYAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGDRVVWHQQKPGKAPKALIYSSSHRYKGVPSRFSGSGS |
| | | GTEFTLTISSLQPEDFATYFCQQFKSYPLTFGQGTKLEIK |

A-H.67

| | | |
|---|---|---|
| SEQ ID NO: | VH + VL (ScFv) | QVQLVQSGAEVKKPGSSVKVSCKASGTDFKLTYIHWVRQAP |
| 1336 | | GQGLEWMGRIFPGSGNVKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVDNRVAWYQQKPGKAPKALIYSSSHRYKGVPSRFSGSGS |
| | | GTEFTLTISSLQPEDFATYFCQQFKSYPLTFGQGTKLEIK |

A-H.68

| | | |
|---|---|---|
| SEQ ID NO: | VH + VL (ScFv) | QVQLVQSGAEVKKPGSSVKVSCKASGHDFRLTYIHWVRQAP |
| 1337 | | GQGLEWMGRISAGSGNVKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVADRVAWYQQKPGKAPKALIYSSSHRYKGVPSRFSGSGS |
| | | GTEFTLTISSLQPEDFATYFCQQFKSYPLTFGQGTKLEIK |

A-H.69 (also referred to as A-H.13)

| | | |
|---|---|---|
| SEQ ID NO: | VH + VL (ScFv) | QVQLVQSGAEVKKPGSSVKVSCKASGTDFKLTYIHWVRQAP |
| 110 | | GQGLEWMGRIFPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNRVAWYQQKPGKAPKALIYSSSHRYKGVPSRFSGSGS |
| | | GTEFTLTISSLQPEDFATYFCQQFKSYPLTFGQGTKLEIK |

A-H humanized-matured VH

| | | |
|---|---|---|
| SEQ ID NO | VH-humanized matured | QVQLVQSGAEVKKPGSSVKVSCKASGTDFKLTYIHWVRQAP |
| 1310 | 1 | GQGLEWMGRIFPGSGNVKYNEKFKGRVTITADTSTSTAYME |
| | | LSSLRSEDTAVYYCAGSYYSYDVLDYWGQGTTVTVSS |
| SEQ ID NO: | VH-humanized matured | QVQLVQSGAEVKKPGSSVKVSCKASGTDFKLTYIHWVRQAP |
| 1311 | 2 | GQGLEWMGRIFPGSGNVKYNEKFKGRVTITADTSTSTAYME |
| | | LSSLRSEDTAVYYCAVSYYSYDVLDYWGQGTTVTVSS |
| SEQ ID NO: | VH-humanized matured | QVQLVQSGAEVKKPGSSVKVSCKASGHDFRLTYIHWVRQAP |
| 1312 | 3 | GQGLEWMGRISAGSGNVKYNEKFKGRVTITADTSTSTAYME |
| | | LSSLRSEDTAVYYCAVSYYSYDVLDYWGQGTTVTVSS |

A-H humanized-matured VL

| | | |
|---|---|---|
| SEQ ID NO: | VL-humanized matured | DIQMTQSPSFLSASVGDRVTITCKASQNVDNRVAWYQQKPG |
| 1313 | 1 | KAPKALIYSSSHRYKGVPSRFSGSGSGTEFTLTISSLQPED |
| | | FATYFCQQFKSYPLTFGQGTKLEIK |
| SEQ ID NO: | VL-humanized matured | DIQMTQSPSFLSASVGDRVTITCKASQNVADRVAWYQQKPG |
| 1314 | 2 | KAPKALIYSSSHRYKGVPSRFSGSGSGTEFTLTISSLQPED |
| | | FATYFCQQFKSYPLTFGQGTKLEIK |

A-H.70

| | | |
|---|---|---|
| SEQ ID NO: | HC CDR1 | GHDFRLTYIH |
| 3650 | | |
| SEQ ID NO: | HC CDR2 | RVSAGSGNVKYNEKFKG |
| 3651 | | |
| SEQ ID NO: 5 | HC CDR3 | SYYSYDVLDY |
| SEQ ID NO: | LC CDR1 | KASQNVGNRVV |
| 3652 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWVRQAP |
| 1346 | (CDRs underlined) | GQGLEWMGRVSAGSGNVKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVGNRVVWHQQKPG |
| 1347 | (CDRs underlined) | KAPKALIYSSSHRYKGVPSRFSGSGSGTEFTLTISSLQPED |
| | | FATYFCQQFKSYPLTFGQGTKLEIK |

A-H.71

| | | |
|---|---|---|
| SEQ ID NO | HC CDR1 | GHDFRLTYIH |
| 3650 | | |
| SEQ ID NO: | HC CDR2 | RIYAGSGNVKYNEKFKG |
| 3654 | | |
| SEQ ID NO: 5 | HC CDR3 | SYYSYDVLDY |
| SEQ ID NO: | LC CDR1 | KASQNVADRVV |
| 3655 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWVRQAP |
| 1348 | (CDRs underlined) | GQGLEWMGRIYAGSGNVKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVADRVVWHQQKPG |
| 1349 | (CDRs underlined) | KAPKALIYSSSHRYKGVPSRFSGSGSGTEFTLTISSLQPED |
| | | FATYFCQQFKSYPLTFGQGTKLEIK |

A-H.72

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVGDRVA |
| 3658 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVGDRVAWHQQKPG |
| 1351 | (CDRs underlined) | KAPKALIYSSSHRYKGVPSRFSGSGSGTEFTLTISSLQPED |
| | | FATYFCQQFKSYPLTFGQGTKLEIK |

A-1.73

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1353 | (CDRs underlined) | KAPKALIYSSSHRYKGVPSRFSGSGSGTEFTLTISSLQPED |
| | | FATYFCQQFKSYPLTFGQGTKLEIK |

A-H.74

| | | |
|---|---|---|
| SEQ ID NO: | HC CDR1 | GHDFRLTYIH |
| 3650 | | |
| SEQ ID NO: | HC CDR2 | RVSAGSGNVKYNEKFKG |
| 3651 | | |
| SEQ ID NO: 5 | HC CDR3 | SYYSYDVLDY |
| SEQ ID NO: | LC CDR1 | KASQNVADRVV |
| 3655 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDERLTYIHWVRQAP |
| 1346 | (CDRs underlined) | GQGLEWMGRVSAGSGNVKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVADRVVWHQQKPG |
| 1349 | (CDRs underlined) | KAPKALIYSSSHRYKGVPSRFSGSGSGTEFTLTISSLQPED |
| | | FATYFCQQFKSYPLTFGQGTKLEIK |

A-H.75

| | | |
|---|---|---|
| SEQ ID NO: | HC CDR1 | GHDFRLTYIH |
| 3650 | | |
| SEQ ID NO: | HC CDR2 | RVYAGSGNTKYNEKFKG |
| 3659 | | |
| SEQ ID NO: 5 | HC CDR3 | SYYSYDVLDY |
| SEQ ID NO: | LC CDR1 | KASQNVEDRVV |
| 3660 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWVRQAP |
| 1356 | (CDRs underlined) | GQGLEWMGRVYAGSGNTKYNEKFKGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCKASQNVEDRVVWHQQKPG |
| 1357 | (CDRs underlined) | KAPKALIYSSSHRYKGVPSRFSGSGSGTEFTLTISSLQPED |
| | | FATYFCQQFKSYPLTFGQGTKLEIK |

A-H.76

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVADRVV |
| 3655 | | |
| SEQ ID NO: | LC CDR2 | SSSHRYK |
| 3653 | | |
| SEQ ID NO: 8 | LC CDR3 | QQFKSYPLT |

1350

| | | |
|---|---|---|
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVADRVVWHQQKPG |
| 1349 | (CDRs underlined) | KAPKALIYSSSHRYKGVPSRFSGSGSGTEFTLTISSLQPED |
| | | FATYFCQQFKSYPLTFGQGTKLEIK |

A-H.77

| | | |
|---|---|---|
| SEQ ID NO: | HC CDR1 | GHDFKLTYIH |
| 3656 | | |
| SEQ ID NO: | HC CDR2 | RISAGSGNTKYNEKFKG |
| 3661 | | |
| SEQ ID NO: 5 | HC CDR3 | SYYSYDVLDY |
| SEQ ID NO: | LC CDR1 | KASQNVGDRVV |
| 3662 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1360 | (CDRs underlined) | GQGLEWMGRISAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVGDRVVWHQQKPG |
| 1361 | (CDRs underlined) | KAPKALIYSSSHRYKGVPSRFSGSGSGTEFTLTISSLQPED |
| | | FATYFCQQFKSYPLTFGQGTKLEIK |

A-H.78

| | | |
|---|---|---|
| SEQ ID NO: | HC CDR1 | GHDFKLTYIH |
| 3656 | | |
| SEQ ID NO: | HC CDR2 | RIYAGSGNTKYNEKFKG |
| 3663 | | |
| SEQ ID NO: 5 | HC CDR3 | SYYSYDVLDY |
| SEQ ID NO: | LC CDR1 | KASQNVGDRVV |
| 3662 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1362 | (CDRs underlined) | GQGLEWMGRIYAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVGDRVVWHQQKPG |
| 1361 | (CDRs underlined) | KAPKALIYSSSHRYKGVPSRFSGSGSGTEFTLTISSLQPED |
| | | FATYFCQQFKSYPLTFGQGTKLEIK |

A-H.79

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | RASQNVDNRLG |
| 3666 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCRASQNVDNRLGWHQQKPG |
| 1365 | (CDRs underlined) | KAPKALIYSSSHRYKGVPSRFSGSGSGTEFTLTISSLQPED |
| | | FATYFCQQFKSYPLTFGQGTKLEIK |

A-H.80

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | AASSLQK |
| 3668 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1367 | (CDRs underlined) | KAPKALIYAASSLQKGVPSRFSGSGSGTEFTLTISSLQPED |
| | | FATYFCQQFKSYPLTFGQGTKLEIK |

A-H.81

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | SSSHRYK |
| 3653 | | |
| SEQ ID NO: | LC CDR3 | LQHNSYPLT |
| 3664 | | |
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1369 | (CDRs underlined) | KAPKALIYSSSHRYKGVPSRFSGSGSGTEFTLTISSLQPED |
| | | FATYFCLQHNSYPLTFGQGTKLEIK |

A-H.82

| | | |
|---|---|---|
| SEQ ID NO: | HC CDR1 | GHDFKLTYIH |
| 3656 | | |
| SEQ ID NO: | HC CDR2 | RVSAGSGNVNYAQKFQG |
| 3665 | | |
| SEQ ID NO: 5 | HC CDR3 | SYYSYDVLDY |
| SEQ ID NO: | LC CDR1 | RASQNVDNRLG |
| 3666 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQA |
| 1370 | (CDRs underlined) | PGQGLEWMGRVSAGSGNVNYAQKFQGRVTITADTSTSTAY |
| | | MELSSLRSEDTAVYYCAVSYYSYDVLDYWGQGTTVTVSS |
| SEQ ID NO: | VL | DIQMTQSPSFLSASVGDRVTITCRASQNVDNRLGWHQQKPG |
| 1365 | (CDRs underlined) | KAPKALIYSSSHRYKGVPSRFSGSGSGTEFTLTISSLQPED |
| | | FATYFCQQFKSYPLTFGQGTKLEIK |

A-H.83

| | | |
|---|---|---|
| SEQ ID NO: | HC CDR1 | GHDFKLTYIH |
| 3656 | | |
| SEQ ID NO: | HC CDR2 | RVSAGSGNVNYAQKFQG |
| 3665 | | |
| SEQ ID NO: S | HC CDR3 | SYYSYDVLDY |
| SEQ ID NO: | LC CDR1 | KASQNVDNRVA |
| 3667 | | |
| SEQ ID NO: | LC CDR2 | AASSLQK |
| 3668 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1370 | (CDRs underlined) | GQGLEWMGRVSAGSGNVNYAQKFQGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1367 | (CDRs underlined) | KAPKALIYAASSLQKGVPSRFSGSGSGTEFTLTISSLQPED |
| | | FATYFCQQFKSYPLTFGQGTKLEIK |

A-H.84

| | | |
|---|---|---|
| SEQ ID NO: | HC CDR1 | GHDFKLTYIH |
| 3656 | | |
| SEQ ID NO: | HC CDR2 | RVSAGSGNVNYAQKFQG |
| 3665 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | SSSHRYK |
| 3653 | | |
| SEQ ID NO: | LC CDR3 | LQHNSYPLT |
| 3664 | | |
| SEQ ID NO: | VH | QVQLVQSGAEVKKPGSSVKVSCKASGHDFKLTYIHWVRQAP |
| 1370 | (CDRs underlined) | GQGLEWMGRVSAGSGNVNYAQKFQGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1369 | (CDRs underlined) | KAPKALIYSSSHRYKGVPSRFSGSGSGTEFTLTISSLQPED |
| | | FATYFCLQHNSYPLTFGQGTKLEIK |

A-H.85

| | | |
|---|---|---|
| SEQ ID NO: | HC CDR1 | GHDFRLTYIH |
| 3650 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVGDRVV |
| 3662 | | |
| SEQ ID NO: | LC CDR2 | SSSHRYK |
| 3653 | | |
| SEQ ID NO: 8 | LC CDR3 | QQFKSYPLT |
| SEQ ID NO: | VH (CDRs underlined) | QVQLVQSGAEVKKPGSSVKVSCKASGHDFRLTYIHWVRQAP |
| 1344 | | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVGDRVVWHQQKPG |
| 1361 | (CDRs underlined) | KAPKALIYSSSHRYKGVPSRFSGSGSGTEFTLTISSLQPED |
| | | FATYFCQQFKSYPLTFGQGTKLEIK |

TABLE 3
Constant region amino acid sequences of human IgG
heavy chains and human kappa light chain

| | | |
|---|---|---|
| Human kappa | LC | RTVAAPSVFI FPPSDEQLKS GTASVVCLLN NFYPREAKVQ WKVDNALQSG |
| constant region | | NSQESVTEQD SKDSTYSLSS TLTLSKADYE KHKVYACEVT HQGLSSPVTK |
| SEQ ID NO: 39 | | SFNRGEC |
| Human | LC | RTVAAPSVFIFPPSDEQLKSGTASVVCLLNNFYPREAKVQWKVDNALQSGNSQ |
| Immunoglobulin | | ESVTEQDSKDSTYSLSSTLTLSKADYEKHKVYACEVTHQGLSSPVTKSFNRGE |
| kappa constant C | | C |
| region | | |
| SEQ ID NO: 3644 | | |
| IgG4 (S228P) | HC | ASTKGPSVFPLAPCSRSTSESTAALGCLVKDYFPEPVTVSWNSGALTSGVHTFP |
| mutant constant | | AVLQSSGLYSLSSVVTVPSSSLGTKTYTCNVDHKPSNTKVDKRVESKYGPPCPP |
| region (EU | | CPAPEFLGGPSVFLFPPKPKDTLMISRTPEVTCVVVDVSQEDPEVQFNWYVDG |
| Numbering) | | VEVHNAKTKPREEQFNSTYRVVSVLTVLHQDWLNGKEYKCKVSNKGLPSSIE |
| SEQ ID NO: 40 | | KTISKAKGQPREPQVYTLPPSQEEMTKNQVSLTCLVKGFYPSDIAVEWESNGQ |
| | | PENNYKTTPPVLDSDGSFFLYSRLTVDKSRWQEGNVFSCSVMHEALHNHYTQ |
| | | KSLSLSLG |
| IgG1 wild type | HC | ASTKGPSVFPLAPSSKSTSGGTAALGCLVKDYFPEPVTVSWNSGALTSGVHTFP |
| SEQ ID NO: 41 | | AVLQSSGLYSLSSVVTVPSSSLGTQTYICNVNHKPSNTKVDKRVEPKSCDKTHT |
| | | CPPCPAPELLGGPSVFLFPPKPKDTLMISRTPEVTCVVVDVSHEDPEVKFNWYV |
| | | DGVEVHNAKTKPREEQYNSTYRVVSVLTVLHQDWLNGKEYKCKVSNKALPAPIE |
| | | KTISKAKGQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALHNHYTQKSL |
| | | SLSPGK |
| IgG1 (N297A) | HC | ASTKGPSVFPLAPSSKSTSGGTAALGCLVKDYFPEPVTVSWNSGALTSGVHTFP |
| mutant constant | | AVLQSSGLYSLSSVVTVPSSSLGTQTYICNVNHKPSNTKVDKRVEPKSCDKTHT |
| region (BU | | CPPCPAPELLGGPSVFLFPPKPKDTLMISRTPEVTCVVVDVSHEDPEVKFNWYV |
| Numbering) | | DGVEVHNAKTKPREEQYASTYRVVSVLTVLHQDWLNGKEYKCKVSNKALPAPIE |
| SEQ ID NO: 42 | | KTISKAKGQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALHNHYTQKSL |
| | | SLSPGK |
| IgM constant | HC | GSASAPTLFPLVSCENSPSDTSSVAVGCLAQDFLPDSITFSWKYKNNSDISSTR |
| delta CDC | | GFPSVLRGGKYAATSQVLLPSKDVMQGTDEHVVCKVQHPNGNKEKNVPLPVIA |
| (P311A, P313S) | | ELPPKVSVFVPPRDGFFGNPRKSKLICQATGFSPRQIQVSWLREGKQVGSGVT |
| SEQ ID NO: 73 | | TDQVQAEAKESGPTTYKVTSTLTIKESDWLGQSMFTCRVDHRGLTFQQNASSM |
| | | CVPDQDTAIRVFAIPPSFASIFLTKSTKLTCLVTDLTTYDSVTISWTRQNGEA |
| | | VKTHTNISESHPNATFSAVGEASICEDDWNSGERFTCTVTHTDLASSLKQTIS |
| | | RPKGVALHRPDVYLLPPAREQLNLRESATITCLVTGFSPADVFVQWMQRGQPL |
| | | SPEKYVTSAPMPEPQAPGRYFAHSILTVSEEEWNTGETYTCVVAHEALPNRVT |
| | | ERTVDKSTGKPTLYNVSLVMSDTAGTCY |
| IgGA1 | HC | ASPTSPKVFPLSLCSTQPDGNVVIACLVQGFFPQEPLSVTWSESGQGVTARNFP |
| SEQ ID NO: 74 | | PSQDASGDLYTTSSQLTLPATQCLAGKSVTCHVKHYTNPSQDVTVPCPVPSTPP |
| | | TPSPSTPPTPSPSCCHPRLSLHRPALEDLLLGSEANLTCTLTGLRDASGVTFTW |
| | | TPSSGKSAVQGPPERDLCGCYSVSSVLPGCAEPWNHGKTFTCTAAYPESKTPLT |
| | | ATLSKSGNTFRPEVHLLPPPSEELALNELVTLTCLARGFSPKDVLVRWLQGSQE |
| | | LPREKYLTWASRQEPSQGTTTFAVTSILRVAAEDWKKGDTFSCMVGHEALPLAF |
| | | TQKTIDRLAGKPTHVNVSVVMAEVDGTCY |
| IgGA2 | HC | ASPTSPKVFPLSLDSTPQDGNVVVACLVQGFFPQEPLSVTWSESGQNVTARNFP |
| SEQ ID NO: 75 | | PSQDASGDLYTTSSQLTLPATQCPDGKSVTCHVKHYTNSSQDVTVPCRVPPPPP |
| | | CCHPRLSLHRPALEDLLLGSEANLTCTLTGLRDASGATFTWTPSSGKSAVQGPP |
| | | ERDLCGCYSVSSVLPGCAQPWNHGETFTCTAAHPELKTPLTANITKSGNTFRPE |
| | | VHLLPPPSEELALNELVTLTCLARGFSPKDVLVRWLQGSQELPREKYLTWASR |
| | | QEPSQGTTTYAVTSILRVAAEDWKKGETFSCMVGHEALPLAFTQKTIDRMAG |
| | | KPTHINVSVVMAEADGTCY |
| Human Ig J chain | HC | MKNHLLFWGVLAVFIKAVHVKAQEDERIVLVDNKCKCARITSRIIRSSEDPNED |
| SEQ ID NO: 76 | | IVERNIRIIVPLNNRENISDPTSPLRTRFVYHLSDLCKKCDPTEVELDNQIVTA |
| | | TQSNICDEDSATETCYTYDRNKCYTAVVPLVYGGETKMVETALTPDACYPD |
| Human | HC | ASTKGPSVFPLAPSSKSTSGGTAALGCLVKDYFPEPVTVSWNSGALTSGVHTFP |
| Immunoglobulin | | AVLQSSGLYSLSSVVTVPSSSLGTQTYICNVNHKPSNTKVDKKVEPKSCDKTHT |
| heavy chain | | CPPCPAPELLGGPSVFLFPPKPKDTLMISRTPEVTCVVVDVSHEDPEVKFNWYV |
| SEQ ID NO: 3645 | | DGVEVHNAKTKPREEQYASTYRVVSVLTVLHQDWLNGKEYKCKVSNKALPAPIE |
| | | KTISKAKGQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALHNHYTQKSL |
| | | SLSPGK |
| Human | HC | ASTKGPSVFPLAPSSKSTSGGTAALGCLVKDYFPEPVTVSWNSGALTSGVHTFP |
| Immunoglobulin | | AVLQSSGLYSLSSVVTVPSSSLGTQTYICNVNHKPSNTKVDKKVEPKSCDKTHT |
| heavy chain | | CPPCPAPELLGGPSVFLFPPKPKDTLMISRTPEVTCVVVDVSHEDPEVKFNWYV |
| SEQ ID NO: 3646 | | DGVEVHNAKTKPREEQYASTYRVVSVLTVLHQDWLNGKEYKCKVSNKALPAPIE |
| | | KTISKAKGQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALHNHYTQKSL |
| | | SLSPGKEPEA |
| Human | HC | ASTKGPSVFPLAPSSKSTSGGTAALGCLVKDYFPEPVTVSWNSGALTSGVHTFP |
| Immunoglobulin | | AVLQSSGLYSLSSVVTVPSSSLGTQTYICNVNHKPSNTKVDKRVEPKSCDKTHT |
| heavy chain | | CPPCPAPELLGGPSVFLFPPKPKDTLMISRTPEVTCVVVDVSHEDPEVKFNWYV |
| SEQ ID NO: 3647 | | DGVEVHNAKTKPREEQYNSTYRVVSVLTVLHQDWLNGKEYKCKVSNKALPAPIE |
| | | KTISKAKGQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALHNHYTQKSL |
| | | SLSPGK |
| Human Fc Knob | HC | DKTHTCPPCPAPELLGGPSVELFPPKPKDTLMISRTPEVTCVVVDVSHEDPEV |
| cys N297A | | KFNWYVDGVEVHNAKTKPREEQYASTYRVVSVLTVLHQDWLNGKEYKCKVSN |
| SEQ ID NO: 3648 | | KALPAPIRKTISKAKGQPREPQVYTLPPCREEMTKNQVSLWCLVKGFYPSDIA |
| | | VEWESNGQPENNYKTTPPVLDSDGSFFLYSKLTVDKSRWQQGNVFSCSVMHE |
| | | ALHNHYTQKSLSLSPGK |
| Human IgG1 hole | HC | ASTKGPSVFPLAPSSKSTSGGTAALGCLVKDYFPEPVTVSWNSGALTSGVHTFP |
| cys N297A | | AVLQSSGLYSLSSVVTVPSSSLGTQTYICNVNHKPSNTKVDKRVEPKSCDKTHT |
| SEQ ID NO: 3649 | | CPPCPAPELLGGPSVFLFPPKPKDTLMISRTPEVTCVVVDVSHEDPEVKFNWYV |
| | | DGVEVHNAKTKPREEQYASTYRVVSVLTVLHQDWLNGKEYKCKVSNKALPAPIE |
| | | KTISKAKGQPREPQVCTLPPSREEMTKNQVSLSCAVKGFYPSDIAVEWESNGQP |
| | | ENNYKTTPPVLDSDGSFFLVSKLTVDKSRWQQGNVFSCSVMHEALHNHYTQKSL |
| | | SLSPGK |

TABLE 4
Exemplary Fc KiH mutations and optional Cysteine mutations

| | Knob | Hole |
|---|---|---|
| Position | Mutation | Mutation |
| T366 | T366W | T366S |
| L368 | — | L368A |
| Y407 | — | Y407V |

Additional Cysteine Mutations to form a stabilizing disulfide bridge

| Position | Knob CH3 | Hole CH3 |
|---|---|---|
| S354 | S354C | — |
| Y349 | — | Y349C |

TABLE 5
CRS grading

| | |
|---|---|
| Gr1 | Supportive care only |
| Gr2 | IV therapies +/− hospitalization. |
| Gr3 | Hypotension requiring IV fluids or low-dose |
| | vasoactives or hypoxemia requiring oxygen, |
| | CPAP, or BIPAP. |
| Gr4 | Hypotension requiring high-dose vasoactives |
| | or hypoxemia requiring mechanical ventilation. |
| Gr 5 | Death |

TABLE 6
CTCAE v 4.0 CRS grading scale

| CRS | |
|---|---|
| grade | Characteristics |
| Grade 1 | Mild; No infusion interruption; No intervention |
| Grade 2 | Infusion interruption indicated but responds |
| | promptly to symptomatic treatment (e.g., |
| | antihistamines, NSAIDS, narcotics, IV fluids); |
| | prophylactic medications indicated for <=24 hrs |
| Grade 3 | Prolonged (e.g., not rapidly responsive to |
| | symptomatic medications and/or brief |
| | interruption of infusion); recurrence of |
| | symptoms following initial improvement; |
| | hospitalization indicated for clinical |
| | sequelae (e.g., renal impairment, pulmonary |
| | infiltrates) |
| Grade 4 | Life threatening consequences; pressor or ventilator support |

TABLE 7
NCI CRS grading scale

| CRS | |
|---|---|
| grade | Characteristics |
| Grade 1 | Symptoms are not life threatening and require |
| | symptomatic treatment only; e.g., fever, |
| | nausea, fatigue, headache, myalgias, malaise |
| Grade 2 | Symptoms require and respond to moderate |
| | intervention; Oxygen requirement <40% or |
| | hypotension responsive to fluids or low |
| | dose pressors or Grade 2 organ toxicity |
| Grade 3 | Symptoms require and respond to aggressive |
| | intervention; Oxygen requirement >=40% or |
| | Hypotension requiring high dose or multiple |
| | pressors or grade 3 organ toxicity or grade 4 |
| | transaminitis |
| Grade 4 | Life threatening symptoms Requirement |
| | for ventilator support or Grade 4; organ toxicity |
| | (excluding transaminitis) |

TABLE 8A
List of TCRβV subfamilies and subfamily members

| Reference | Subfamily | Subfamily members |
|---|---|---|
| A | TCRβ V6 | TCRβ V6-4*01, TCRβ V6-4*02, |
| | Also referred | TCRβ V6-9*01, TCRβ V6-8*01, |
| | to as: | TCRβ V6-5*01, TCRβ V6-6*02, |
| | TCR VB 13.1 | TCRβ V6-6*01, TCRβ V6-2*01, |
| | | TCRβ V6-3*01 or TCRβ V6-1*01. |
| B | TCRβ V10 | TCRβ V10-1*01, TCRβ V10-1*02, |
| | Also referred | TCRβ V10-3*01 or TCRβ |
| | to as: | V10-2*01 |
| | TCRβ V12 | |
| C | TCRβ V12 | TCRβ V12-4*01, TCRβ V12-3*01, |
| | Also referred | or TCRβ V12-5*01 |
| | to as: | |
| | TCRβ V8.1 | |
| D | TCRβ V5 | TCRβ V5-5*01, TCRβ V5-6*01, |
| | | TCRβ V5-4*01, TCRβ V5-8*01, |
| | | TCRβ V5-1*01 |
| E | TCRβ V7 | TCRβ V7-7*01, TCRβ V7-6*01, |
| | | TCRβ V7-8*02, TCRβ V7- |
| | | 4*01, TCRβ V7-2*02, TCRβ |
| | | V7-2*03, TCRβ V7-2*01, TCRβ |
| | | V7-3*01, TCRβ V7-9*03, |
| | | or TCRβ V7-9*01 |
| F | TCRβ VII | TCRβ V11-1*01, TCRβ |
| | | V11-2*01 or TCRβ V11-3*01 |
| G | TCRβ V14 | TCRβ V14*01 |
| H | TCRβ V16 | TCRβ V16*01 |
| I | TCRβ V18 | TCRβ V18*01 |
| J | TCRβ V9 | TCRβ V9*01 or TCRβ V9*02 |
| K | TCRβ V13 | TCRβ V13*01 |
| L | TCRβ V4 | TCRβ V4-2*01, TCRβ V4-3*01, |
| | | or TCRβ V4-1*01 |
| M | TCRβ V3 | TCRβ V3-1*01 |
| N | TCRβ V2 | TCRβ V2*01 |
| O | TCRβ V15 | TCRβ V15*01 |
| P | TCRβ V30 | TCRβ V30*01, or TCRβ V30*02 |
| Q | TCRβ V19 | TCRβ V19*01, or TCRβ V19*02 |
| R | TCRβ V27 | TCRβ V27*01. |
| S | TCRβ V28 | TCRβ V28*01. |
| | TCRβ V24 | TCRβ V24-1*01 |
| U | TCRβ V20 | TCRβ V20-1*01, or TCRβ V20-1*02 |
| V | TCRβ V25 | TCRβ V25-1*01 |
| W | TCRβ V29 | TCRβ V29-1*01 |

TABLE 8B
Additional TCRβV subfamilies
Subfamily
TCRβ V1
TCRβ V17
TCRβ V21
TCRβ V23
TCRβ V26

TABLE 10A
Exemplary anti-TCRβV antibody molecules

| | | | | |
|---|---|---|---|---|
| TRBV6-1 | TRBV6-1*01 | BAM13 | Pierce Endogen | Mouse |
| | | (TRBV6-1, | V beta 13 | IgG1 |
| | | TRBV6-5) | BD Biosciences | |
| | | | Vbeta13.1, | |
| | | | 13.3 | |
| TRBV6-2 | TRBV6-2*01 | H132 | Coulter Vbeta13.2 | Mouse |
| TRBV6-3 | TRBV6-3*01 | | | IgG1 |
| TRBV6-4 | TRBV6-4*01 | | | |
| | TRBV6-4*02 | | | |
| TRBV6-5 | TRBV6-5*01 | IMMU 222 | Serotec V | Mouse |
| | | (TRBV6-5, | BETA 13.1 | IgG2b |
| | | TRBV6-6 and | Coulter Vbeta13.1 | |
| | | TRBV6-9) | Pierce Endogen | Mouse |
| | | BAM13 | V beta 13 | IgG1 |
| | | (TRBV6-1, | BD Biosciences | |
| | | TRBV6-5) | Vbeta13.1, | |
| | | | 13.3 | |
| TRBV6-6 | TRBV6-6*01 | JU-74 (TRBV6-6) | Serotec Vbeta 13.6 | Mouse |
| | TRBV6-6*02 | JU74.3 | Coulter Vbeta13.6 | IgG1 |
| | TRBV6-6*03 | (TRBV6-6) | | |
| | TRBV6-6*04 | IMMU 222 | Serotec V | Mouse |
| | TRBV6-6*05 | (TRBV6-5, | BETA 13.1 | IgG2b |
| | | TRBV6-6 and | Coulter Vbeta13.1 | |
| TRBV6-8 | TRBV6-8*01 | TRBV6-9) | | |
| TRBV6-9 | TRBV6-9*01 | IMMU 222 | Serotec V | Mouse |
| | | (TRBV6-5, | BETA 13.1 | lgG2b |
| | | TRBV6-6 and | Coulter Vbeta13.1 | |
| | | TRBV6-9) | | |

TABLE 12
Amino acid sequences for anti TCRβ V10 antibodies. Amino acid and nucleotide sequences
for murine and humanized antibody molecules which bind to TCRBV 10 (e.g., TCRBV 10-1,
TCRBV 10-2 or TCRBV 10-3). The amino acid the heavy and light chain CDRs, and the
amino acid and nucleotide sequences of the heavy and light chain variable regions,
and the heavy and light chains are shown.
Murine antibody D, also referred to as S511 antibody

| | | |
|---|---|---|
| SEQ ID NO: 1288 | HC CDR1 (Kabat) | SYGMS |
| SEQ ID NO: 1289 | HC CDR2 (Kabat) | LISSGGSYTYYTDSVKG |
| SEQ ID NO: 1290 | HC CDR3 (Kabat) | HGGNFFDY |
| SEQ ID NO: 1291 | HC CDR1 (Chothia) | GFTFRSY |
| SEQ ID NO: 1292 | HC CDR2 (Chothia) | SSGGSY |
| SEQ ID NO: 1290 | HC CDR3 (Chothia) | HGGNFFDY |
| SEQ ID NO: 1293 | HC CDR1 (Combined) | GFTFRSYGMS |
| SEQ ID NO: 1289 | HC CDR2 (Combined)) | LISSGGSYTYYTDSVKG |
| SEQ ID NO: 1290 | HC CDR3 (Combined) | HGGNFFDY |
| SEQ ID NO: 1294 | LC CDR1 (Kabat) | SVSSSVSYMH |
| SEQ ID NO: 1295 | LC CDR2 (Kabat) | DTSKLAS |
| SEQ ID NO: 1296 | LC CDR3 (Kabat) | QQWSSNPQYT |
| SEQ ID NO: 1297 | LC CDR1 (Chothia) | SSSVSY |
| SEQ ID NO: 1295 | LC CDR2 (Chothia) | DTSKLAS |
| SEQ ID NO: 1296 | LC CDR3 (Chothia) | QQWSSNPQYT |
| SEQ ID NO: 1294 | LC CDR1 (Combined) | SVSSSVSYMH |
| SEQ ID NO: 1295 | LC CDR2 (Combined) | DTSKLAS |
| SEQ ID NO: 1296 | LC CDR3 (Combined) | QQWSSNPQYT |
| SEQ ID NO: 3183 | VH | EVQLVESGGDLVKPGGSLKLSCAVSGFTFRSYGMSWVR |
| | | QTPDKRLEWVALISSGGSYTYYTDSVKGRFTISRDNAKN |
| | | TLYLQMSSLKSEDTAIYYCSRHGGNFFDYWGQGTTLTVS |
| | | S |
| SEQ ID NO: 3184 | VL | QIVLTQSPSIMSASPGEKVTMTCSVSSSVSYMHWYQQKS |
| | | GTSPKRWIYDTSKLASGVPARFSGSGSGTSYSLTISSMEA |
| | | EDAATYYCQQWSSNPQYTFGGGTKLEIK |

Humanized antibody D (D-H antibody)
Variable light chain (VL)

| | | | |
|---|---|---|---|
| SEQ ID NO: 3185 | VL | D-H.1 | DIVLTQSPAFLSVTPGEKVTITCSVSSSVSYMHWYQQKPDQAPK |
| | | | LLIYDTSKLASGVPSRFSGSGSGTDYTFTISSLEAEDAATYYCQQ |
| | | | WSSNPQYTFGQGTKLEIK |
| SEQ ID NO: 3186 | VL | D-H.2 | AIQLTQSPSSLSASVGDRVTITCSVSSSVSYMHWYQQKPGKAPK |
| | | | LLIYDTSKLASGVPSRFSGSGSGTDYTLTISSLQPEDFATYYCQQ |
| | | | WSSNPQYTFGQGTKLEIK |
| SEQ ID NO: 3187 | VL | D-H.3 | DIQLTQSPSFLSASVGDRVTITCSVSSSVSYMHWYQQKPGKAPK |
| | | | LLIYDTSKLASGVPSRFSGSGSGTEYTLTISSLQPEDFATYYCQQ |
| | | | WSSNPQYTFGQGTKLEIK |
| SEQ ID NO: 3188 | VL | D-H.4 | DIQLTQSPSSLSASVGDRVTITCSVSSSVSYMHWYQQKPGKAPK |
| | | | LLIYDTSKLASGVPSRFSGSGSGTDYTLTISSLQPEDFATYYCQQ |
| | | | WSSNPQYTFGQGTKLEIK |
| SEQ ID NO: 3189 | VL | D-H.5 | DIQLTQSPSSVSASVGDRVTITCSVSSSVSYMHWYQQKPGKAPK |
| | | | LLIYDTSKLASGVPSRFSGSGSGTDYTLTISSLQPEDFATYYCQQ |
| | | | WSSNPQYTFGQGTKLEIK |
| SEQ ID NO: 3190 | VL | D-H.6 | DIQLTQSPSSLSASVGDRVTITCSVSSSVSYMHWYQQKPGKVPK |
| | | | LLIYDTSKLASGVPSRFSGSGSGTDYTLTISSLQPEDVATYYCQQ |
| | | | WSSNPQYTFGQGTKLEIK |
| SEQ ID NO: 3191 | VL | D-H.7 | DIQLTQSPSSLSASVGDRVTITCSVSSSVSYMHWYQQKPGQAPK |
| | | | LLIYDTSKLASGVPSRFSGSGSGTDYTLTISSLQPEDVATYYCQQ |
| | | | WSSNPQYTFGQGTKLEIK |
| SEQ ID NO: 3192 | VL | D-H.8 | EIVLTQSPDFQSVTPKEKVTITCSVSSSVSYMHWYQQKPDQSPKL |
| | | | LIYDTSKLASGVPSRFSGSGSGTDYTLTINSLEAEDAATYYCQQ |
| | | | WSSNPQYTFGQGTKLEIK |
| SEQ ID NO: 3193 | VL | D-H.9 | AIRLTQSPFSLSASVGDRVTITCSVSSSVSYMHWYQQKPAKAPK |
| | | | LFIYDTSKLASGVPSRFSGSGSGTDYTLTISSLQPEDFATYYCQQ |
| | | | WSSNPQYTFGQGTKLEIK |
| SEQ ID NO: 3194 | VL | D- | DIQLTQSPSSLSASVGDRVTITCSVSSSVSYMHWYQQKPGKAPK |
| | | H.10 | LLIYDTSKLASGVPSRFSGSGSGTDYTFTISSLQPEDIATYYCQQ |
| | | | WSSNPQYTFGQGTKLEIK |
| SEQ ID NO: 3195 | VL | D- | EIVLTQSPATLSLSPGERATLSCSVSSSVSYMHWYQQKPGQAPK |
| | | H.11 | LLIYDTSKLASGIPARFSGSGSGTDYTLTISSLEPEDFAVYYCQQ |
| | | | WSSNPQYTFGQGTKLEIK |
| SEQ ID NO: 3196 | VL | D- | DIQLTQSPSTLSASVGDRVTITCSVSSSVSYMHWYQQKPGKAPK |
| | | H.12 | LLIYDTSKLASGVPSRFSGSGSGTEYTLTISSLQPDDFATYYCQQ |
| | | | WSSNPQYTFGQGTKLEIK |
| SEQ ID NO: 3197 | VL | D- | DIQLTQSPSSLSASVGDRVTITCSVSSSVSYMHWYQQKPGKTPK |
| | | H.13 | LLIYDTSKLASGIPSRFSGSGSGTDYTLTIRSLQPEDFATYYCQQ |
| | | | WSSNPQYTFGQGTKLEIK |
| SEQ ID NO: 3198 | VL | D- | EIVLTQSPPTLSLSPGERVTLSCSVSSSVSYMHWYQQKPGQAPKL |
| | | H.14 | LIYDTSKLASGIPARFSGSGSGTDYTLTISSLQPEDFAVYYCQQW |
| | | | SSNPQYTFGQGTKLEIK |
| SEQ ID NO: 3199 | VL | D- | DIQLTQSPSSLSASVGDRVTITCSVSSSVSYMHWYQQKPGKAPK |
| | | H.15 | RLIYDTSKLASGVPSRFSGSGSGTEYTLTISSLQPEDFATYYCQQ |
| | | | WSSNPQYTFGQGTKLEIK |
| SEQ ID NO: 3200 | VL | D- | EIVLTQSPATLSLSPGERATLSCSVSSSVSYMHWYQQKPGQAPK |
| | | H.16 | LLIYDTSKLASGIPARFSGSGPGTDYTLTISSLEPEDFAVYYCQQ |
| | | | WSSNPQYTFGQGTKLEIK |
| SEQ ID NO: 3201 | VL | D- | EIVLTQSPATLSLSPGERATLSCSVSSSVSYMHWYQQKPGQAPK |
| | | H.17 | LLIYDTSKLASGIPARFSGSGSGTDYTLTISRLEPEDFAVYYCQQ |
| | | | WSSNPQYTFGQGTKLEIK |
| SEQ ID NO: 3202 | VL | D- | EIVLTQSPATLSLSPGERATLSCSVSSSVSYMHWYQQKPGQAPK |
| | | H.18 | LLIYDTSKLASGIPARFSGSGSGTDYTLTISSLQPEDFAVYYCQQ |
| | | | WSSNPQYTFGQGTKLEIK |
| SEQ ID NO: 3203 | VL | D- | EIVLTQSPATLSVSPGERATLSCSVSSSVSYMHWYQQKPGQAPK |
| | | H.19 | LLIYDTSKLASGIPARFSGSGSGTEYTLTISSLQSEDFAVYYCQQ |
| | | | WSSNPQYTFGQGTKLEIK |
| SEQ ID NO: 3204 | VL | D- | EIVLTQSPATLSVSPGERATLSCSVSSSVSYMHWYQQKPGQAPK |
| | | H.20 | LLIYDTSKLASGIPARFSGSGSGTEYTLTISILQSEDFAVYYCQQW |
| | | | SSNPQYTFGQGTKLEIK |
| SEQ ID NO: 3205 | VL | D- | EIVLTQSPPTLSLSPGERVTLSCSVSSSVSYMHWYQQKPGQAPKL |
| | | H.2 | LIYDTSKLASSIPARFSGSGSGTDYTLTISSLQPEDFAVYYCQQWS |
| | | | SNPQYTFGQGTKLEIK |
| SEQ ID NO: 3206 | VL | D- | DIQLTQSPSSLSASVGDRVTITCSVSSSVSYMHWYQQKPGKAPK |
| | | H.22 | SLIYDTSKLASGVPSRFSGSGSGTDYTLTISSLQPEDFATYYCQQ |
| | | | WSSNPQYTFGQGTKLEIK |
| SEQ ID NO: 3207 | VI | D- | DIQLTQSPSSLSASVGDRVTITCSVSSSVSYMHWYQQKPGKAPK |
| | | H.23 | RLIYDTSKLASGVPSRFSGSGSGTEYTLTISNLQPEDFATYYCQQ |
| | | | WSSNPQYTFGQGTKLEIK |
| SEQ ID NO: 3208 | VL | D- | DIQLTQSPSAMSASVGDRVTITCSVSSSVSYMHWYQQKPGKVPK |
| | | H.24 | RLIYDTSKLASGVPSRFSGSGSGTEYTLTISSLQPEDFATYYCQQ |
| | | | WSSNPQYTFGQGTKLEIK |
| SEQ ID NO: 3209 | VL | D. | EIVLTQSPATLSLSPGERATLSCSVSSSVSYMHWYQQKPGQAPK |
| | | H.25 | LLIYDTSKLASGIPDRESGSGSGTDYTLTISRLEPEDFAVYYCQQ |
| | | | WSSNPQYTFGQGTKLEIK |
| SEQ ID NO: 3210 | VL | D- | EIVLTQSPATLSLSPGERATLSCSVSSSVSYMHWYQQKPGLAPKL |
| | | H.26 | LIYDTSKLASGIPDRFSGSGSGTDYTLTISRLEPEDFAVYYCQQW |
| | | | SSNPQYTFGQGTKLEIK |
| SEQ ID NO: 3211 | VL | D- | EIVLTQSPGTLSLSPGERATLSCSVSSSVSYMHWYQQKPGQAPK |
| | | H.27 | LLIYDTSKLASGIPDRFSGSGSGTDYTLTISRLEPEDFAVYYCQQ |
| | | | WSSNPQYTFGQGTKLEIK |
| SEQ ID NO: 3212 | VL | D- | DIQLTQSPSSLSASVGDRVTITCSVSSSVSYMHWYQQKPGKAPK |
| | | H.28 | SLIYDTSKLASGVPSKFSGSGSGTDYTLTISSLQPEDFATYYCQQ |
| | | | WSSNPQYTFGQGTKLEIK |
| SEQ ID NO: 3213 | VL | D- | DIQLTQSPSSLSASVGDRVTITCSVSSSVSYMHWYQQKPEKAPKS |
| | | H.29 | LIYDTSKLASGVPSRFSGSGSGTDYTLTISSLQPEDFATYYCQQW |
| | | | SSNPQYTFGQGTKLEIK |
| SEQ ID NO: 3214 | VL | D- | DIVLTQSPDSLAVSLGERATINCSVSSSVSYMHWYQQKPGQPPK |
| | | H.30 | LLIYDTSKLASGVPDRESGSGSGTDYTLTISSLQAEDVAVYYCQQ |
| | | | WSSNPQYTFGQGTKLEIK |
| SEQ ID NO: 3215 | VL | D- | EIVLTQTPLSLSITPGEQASMSCSVSSSVSYMHWYLQKARPVPKL |
| | | H.31 | LIYDTSKLASGVPDRFSGSGSGTDYTLKISRVEAEDFGVYYCQQ |
| | | | WSSNPQYTFGQGTKLEIK |
| SEQ ID NO: 3216 | VL | D- | EIVLTQTPLSLSITPGEQASISCSVSSSVSYMHWYLQKARPVPKLL |
| | | H.32 | IYDTSKLASGVPDRFSGSGSGTDYTLKISRVEAEDFGVYYCQQW |
| | | | SSNPQYTFGQGTKLEIK |
| SEQ ID NO: 3217 | VL | D- | DIVLTQSPLSLPVTPGEPASISCSVSSSVSYMHWYLQKPGQSPKL |
| | | H.33 | LIYDTSKLASGVPDRFSGSGSGTDYTLKISRVEAEDVGVYYCQQ |
| | | | WSSNPQYTFGQGTKLEIK |
| SEQ ID NO: 3218 | VL | D- | DIVLTQSPLSLPVTLGQPASISCSVSSSVSYMHWYQQRPGQSPKR |
| | | H.34 | LIYDTSKLASGVPDRESGSGSGTDYTLKISRVEAEDVGVYYCQQ |
| | | | WSSNPQYTFGQGTKLEIK |
| SEQ ID NO: 3219 | VL | D- | DIVLTQTPLSLPVTPGEPASISCSVSSSVSYMHWYLQKPGQSPKL |
| | | H.35 | LIYDTSKLASGVPDRFSGSGSGTDYTLKISRVEAEDVGVYYCQQ |
| | | | WSSNPQYTFGQGTKLEIK |
| SEQ ID NO: 3220 | VL | D- | DIVLTQTPLSLSVTPGQPASISCSVSSSVSYMHWYLQKPGQSPKL |
| | | H.36 | LIYDTSKLASGVPDRFSGSGSGTDYTLKISRVEAEDVGVYYCQQ |
| | | | WSSNPQYTFGQGTKLEIK |
| SEQ ID NO: 3221 | VL | D- | DIVLTQTPLSLSVTPGQPASISCSVSSSVSYMHWYLQKPGQPPKL |
| | | H.37 | LIYDTSKLASGVPDRFSGSGSGTDYTLKISRVEAEDVGVYYCQQ |
| | | | WSSNPQYTFGQGTKLEIK |
| SEQ ID NO: 3222 | VL | D- | DIQLIQSPSFLSASVGDRVSIICSVSSSVSYMHWYLQKPGKSPKLF |
| | | H.38 | IYDTSKLASGVSSRFSGRGSGTDYTLTIISLKPEDFAAYYCQQWS |
| | | | SNPQYTFGQGTKLEIK |
| SEQ ID NO: 3223 | VL | D- | DIVLTQTPLSSPVTLGQPASISCSVSSSVSYMHWYQQRPGQPPKL |
| | | H.39 | LIYDTSKLASGVPDRFSQSGAGTDYTLKISRVEAEDVGVYYCQQ |
| | | | WSSNPQYTFGQQTKLEIK |
| SEQ ID NO: 3224 | VL | D- | EITLTQSPAFMSATPGDKVNISCSVSSSVSYMHWYQQKPGEAPK |
| | | H.40 | FIIYDTSKLASGIPPRFSGSGYGTDYTLTINNIESEDAAYYYCQQW |
| | | | SSNPQYTFGQGTKLEIK |

Variable HEAVY chain (VH)

| | | | |
|---|---|---|---|
| SEQ ID NO: 3225 | VH | D-H.1 | EVQLVESGGGLVKPGGSLRLSCAVSGFTFRSYGMSWVRQAPGK |
| | | | GLEWVALISSGGSYTYYTDSVKGRFTISRDNSKNTLYLQMNSLK |
| | | | TEDTAVYYCSRHGGNFFDYWGQGTTVTVSS |
| SEQ ID NO: 3226 | VH | D-H.2 | EVQLVESGGALVKPGGSLRLSCAVSGFTFRSYGMSWVRQAPGK |
| | | | GLEWVALISSGGSYTYYTDSVKGRFTISRDNSKNTLYLQMNSLK |
| | | | TEDTAVYYCSRHGGNFFDYWGQGTTVTVSS |
| SEQ ID NO: 3227 | VH | D-H.3 | EVQLVESGGGLVQPGGSLRLSCAVSGFTFRSYGMSWVRQAPGK |
| | | | GLEWVALISSGGSYTYYTDSVKGRFTISRDNAKNTLYLQMNSLR |
| | | | AEDTAVYYCSRHGGNFFDYWGQGTTVTVSS |
| SEQ ID NO: 3228 | VH | D-H.4 | EVQLVESGGGLVQPGGSLRLSCAVSGFTFRSYGMSWVRQAPGK |
| | | | GLEWVALISSGGSYTYYTDSVKGRFTISRDNAKNSLYLQMNSLR |
| | | | AEDTAVYYCSRHGGNFFDYWGQGTTVTVSS |
| SEQ ID NO: 3229 | VH | D-H.5 | EVQLVESGGGLVQPGGSLRLSCAVSGFTFRSYGMSWVRQAPGK |
| | | | GLEWVALISSGGSYTYYTDSVKGRFTISRDNSKNSLYLQMNSLK |
| | | | TEDTAVYYCSRHGGNFFDYWGQGTTVTVSS |
| SEQ ID NO: 3230 | VH | D-H.6 | EVQLVESGGGLVQPGGSLRLSCAVSGFTFRSYGMSWVRQAPGK |
| | | | GLEWVALISSGGSYTYYTDSVKGRFTISRDNAKNSLYLQMNSLR |
| | | | AEDMAVYYCSRHGGNFFDYWGQGTTVTVSS |
| SEQ ID NO: 3231 | VH | D-H.7 | EVQLVESGGGLVQPGGSLRLSCAVSGFTFRSYGMSWVRQAPGK |
| | | | GLEWVALISSGGSYTYYTDSVKGQFTISRDNAKNTLYLQMNSLR |
| | | | AEDMAVYYCSRHGGNFFDYWGQGTTVTVSS |
| SEQ ID NO: 3232 | VH | D-H.8 | EVQLVESGGGLVKPGRSLRLSCTVSGFTFRSYGMSWVRQAPGK |
| | | | GLEWVALISSGGSYTYYTDSVKGRFTISRDNSKNILYLQMNSLK |
| | | | TEDTAVYYCSRHGGNFFDYWGQGTTVTVSS |
| SEQ ID NO: 3233 | VH | D-H.9 | EVQLVESGGGLVKPGGSLRLSCAVSQFTFRSYGMSWVRQAPGK |
| | | | GLEWVALISSGGSYTYYTDSVKGRFTISRDNAKNSLYLQMNSLR |
| | | | AEDTAVYYCSRHQGNFFDYWGQGTTVTVSS |
| SEQ ID NO: 3234 | VH | D- | EVQLVESGGGLVQPGGSLKLSCAVSGFTFRSYGMSWVRQASGK |
| | | H.10 | GLEWVALISSGGSYTYYTDSVKGRFTISRDNSKNTLYLQMNSLK |
| | | | TEDTAVYYCSRHGGNFFDYWGQGTTVTVSS |
| SEQ ID NO: 3235 | VH | D- | QVQLVESGGGVVQPGGSLRLSCAVSGFTFRSYGMSWVRQAPGK |
| | | H.11 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3236 | VH | D- | QVQLVESGGGVVQPGRSLRLSCAVSGFTFRSYGMSWVRQAPGK |
| | | H.12 | GLEWVALISSGGSYTYYTDSVKGRFTISRDNSKNTLYLQMSSLR |
| | | | AEDTAVYYCSRHGGNFFDYWGQGTTVTVSS |
| SEQ ID NO: 3237 | VH | D- | EVQLVESGGGLVQPGGSLRLSCPVSGFTFRSYGMSWVRQAPQK |
| | | H.13 | GLEWVALISSGGSYTYYTDSVKGRFTISRDNANNSLYLQMNSLR |
| | | | AEDTAVYYCSRHGGNFFDYWGQGTTVTVSS |
| SEQ ID NO: 3238 | VH | D- | EVQLVESGGGLVQPGRSLRLSCTVSGFTFRSYGMSWVRQAPGK |
| | | H.14 | GLEWVALISSGGSYTYYTDSVKGRFTISRDNSKNILYLQMNSLK |
| | | | TEDTAVYYCSRHGGNFFDYWGQGTTVTVSS |
| SEQ ID NO: 3239 | VH | D- | EVQLVESGGGLVQPGPSLRLSCTVSGFTFRSYGMSWVRQAPGK |
| | | H.15 | GLEWVALISSGGSYTYYTDSVKGRFTISRDNSKNILYLQMNSLK |
| | | | TEDTAVYYCSRHGGNFFDYWGQGTTVTVSS |
| SEQ ID NO: 3240 | VH | D- | EVQLVESGGGLVQPGGSLRLSCAVSGFTFRSYGMSWVRQAPGK |
| | | H.16 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3241 | VH | D- | EVQLVESGGGLVQPGGSLRLSCAVSGFTFRSYGMSWVRQAPGK |
| | | H.17 | GLEWVALISSGGSYTYYTDSVKGRFTISRDNAKNSLYLQMNSLR |
| | | | DEDTAVYYCSRHGGNFFDYWGQGTTVTVSS |
| SEQ ID NO: 3242 | VH | D- | QVQLVESGGGLVKPGGSLRLSCAVSGFTFRSYGMSWVRQAPGK |
| | | H.18 | GLEWVALISSGGSYTYYTDSVKGRFTISRDNAKNSLYLQMNSLR |
| | | | AEDTAVYYCSRHQGNFFDYWGQGTTVTVSS |
| SEQ ID NO: 3243 | VH | D- | QVQLVESGGGVVQPGRSLRLSCAVSGFTFRSYGMSWVRQAPGK |
| | | H.19 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3244 | VH | D- | EVQLLESGGGLVQPGGSLRLSCAVSGFTFRSYGMSWVRQAPGK |
| | | H.20 | GLEWVALISSGGSYTYYTDSVKQRFTISRDNSKNTLYLQMNSLR |
| | | | AEDTAVYYCSRHGGNFFDYWGQGTTVTVSS |
| SEQ ID NO: 3245 | VH | D- | EVQLVESGGGLVQPGGSLRLSCAVSGFTFRSYGMSWVRQAPGK |
| | | H.21 | GLEWVALISSGGSYTYYTDSVKGRFTISRHNSKNTLYLQMNSLR |
| | | | ABDTAVYYCSRHGGNFFDYWGQGTTVTVSS |
| SEQ ID NO: 3246 | VH | D- | EVQLVESGGGLIQPGGSLRLSCAVSGFTFRSYGMSWVRQPPGKG |
| | | H.22 | LEWVALISSGGSYTYYTDSVKGRFTISRDNSKNTLYLQMNSLRA |
| | | | EDTAVYYCSRHGGNFFDYWGQGTTVTVSS |
| SEQ ID NO: 3247 | VH | D- | EVQLVESGGGLIQPGGSLRLSCAVSGFTFRSYGMSWVRQAPGK |
| | | H.23 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3248 | VH | D- | EVQLVESGGGLVQPGRSLRLSCAVSGFTFRSYGMSWVRQAPGK |
| | | H.24 | GLEWVALISSGGSYTYYTDSVKGRFTISRDNAKNSLYLQMNSLR |
| | | | AEDTALYYCSRHGGNFFDYWGQGTTVTVSS |
| SEQ ID NO: 3249 | VH | D- | QVQLVESGGGVVQPQRSLRLSCAVSGFTFRSYGMSWVRQAPGK |
| | | H.25 | GLEWVALISSGGSYTYYTDSVKGRFTISRDNSKNRLYLQMNSLR |
| | | | AEDTAVYYCSRHGGNFFDYWGQGTTVTVSS |
| SEQ ID NO: 3250 | VH | D- | QVQLVESGGGVVQPGRSLRLSCAVSGFTFRSYGMSWVRQAPGK |
| | | H.26 | GLEWVALISSGGSYTYYTDSVKGRFTISRDNSKNTLYLQMNSLR |
| | | | AEGTAVYYCSRHGGNFFDYWGQGTTVTVSS |
| SEQ ID NO: 3251 | VH | D- | QVQLVESGGGVVQPGRSLRLSCAVSGFTFRSYGMSWVRQAPGK |
| | | H.27 | GLEWVALISSGGSYTYYTDSVKGRFAISRDNSKNTLYLQMNSLR |
| | | | AEDTAVYYCSRHGGNFFDYWGQGTTVTVSS |
| SEQ ID NO: 3252 | VH | D- | QVQLVDSGGGVVQPGRSLRLSCAVSGFTFRSYGMSWVRQAPG |
| | | H.28 | KGLEWVALISSGGSYTYYTDSVKGRFTISRDNSKNTLYLQMNSL |
| | | | RAEDTAVYYCSRHGGNFFDYWGQGTTVTVSS |
| SEQ ID NO: 3253 | VH | D- | EVQLVESGGGVVRPGGSLRLSCAVSGFTFRSYGMSWVRQAPGK |
| | | H.29 | GLEWVALISSGGSYTYYTDSVKGRFTISRDNAKNSLYLQMNSLR |
| | | | AEDTALYHCSRHGGNFFDYWGQGTTVTVSS |
| SEQ ID NO: 3254 | VH | D- | EVQLVESGGVVVQPGGSLRLSCAVSGFTFRSYGMSWVRQAPGK |
| | | H.30 | GLEWVALISSGGSYTYYTDSVKGRFTISRDNSKNSLYLQMNSLR |
| | | | AEDTALYYCSRHGGNFFDYWGQGTTVTVSS |
| SEQ ID NO: 3255 | VH | D- | EVQLVESGGGVVQPGGSLRLSCAVSGFTFRSYGMSWVRQAPGK |
| | | H.31 | GLEWVALISSGGSYTYYTDSVKGRFTISRDNSKNSLYLQMNSLR |
| | | | TEDTALYYCSRHGGNFFDYWGQGTTVTVSS |
| SEQ ID NO: 3256 | VH | D- | EVQLVESGGVVVQPGGSLRLSCAVSGFTFRSYGMSWVRQAPGK |
| | | H.32 | GLEWVALISSGGSYTYYTDSVKGRFTISRDNSKNSLYLQMNSLR |
| | | | TEDTALYYCSRHGGNFFDYWGQGTTVTVSS |
| SEQ ID NO: 3257 | VH | D- | EVQLVETGGGLIQPGGSLRLSCAVSGFTFRSYGMSWVRQAPGK |
| | | H.33 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3258 | VH | D- | EVQLVESGGGLVQPGGSLRLSCAVSGFTFRSYGMSWVRQATGK |
| | | H.34 | GLEWVALISSGGSYTYYTDSVKGRFTISRENAKNSLYLQMNSLR |
| | | | AGDTAVYYCSRHGGNFFDYWGQGTTVTVSS |
| SEQ ID NO: 3259 | VH | D- | EVQLVESRGVLVQPGGSLRLSCAVSGFTFRSYGMSWVRQAPGK |
| | | H.35 | GLEWVALISSGGSYTYYTDSVKGRFTISRDNSKNTLHLQMNSLR |
| | | | AEDTAVYYCSRHGGNFFDYWGQGTTVTVSS |
| SEQ ID NO: 3260 | VH | D- | EVQLVESGGGLVQPGRSLRLSCAVSGFTFRSYGMSWVRQAPGK |
| | | H.36 | GLEWVALISSGGSYTYYTDSVKGRFTISRDNAKNSLYLQMNSLR |
| | | | AEDMALYYCSRHGGNFFDYWGQGTTVTVSS |
| SEQ ID NO: 3261 | VH | D- | QVQLVESGGGLVQPGGSLRLSCSVSGFTFRSYGMSWVRQAPGK |
| | | H.37 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3262 | VH | D- | EVQLVESGGGLVQPGGSLRLSCSVSGFTFRSYGMSWVRQAPGK |
| | | H.38 | GLEWVALISSGGSYTYYTDSVKQRFTISRDNSKNTLYLQMSSLR |
| | | | AEDTAVYYCSRHQGNFFDYWGQGTTVTVSS |
| SEQ ID NO: 3263 | VH | D- | QVQLVESGGGVVQPGRSLRLSCAVSGFTFRSYGMSWVRQAPGK |
| | | H.39 | GLEWVALISSGGSYTYYTDSVKGRFTISRDNSTNTLFLQMNSLR |
| | | | AEDTAVYYCSRHGGNFFDYWGQGTTVTVSS |
| SEQ ID NO: 3264 | VH | D- | QVQLLESGGGLVKPQGSLRLSCAVSGFTFRSYGMSWVRQAPGK |
| | | H.40 | GLEWVALISSGGSYTYYTDSVKGRFTISRDNAKNSLYLQMNSLR |
| | | | AEDTAVYYCSRHGGNFFDYWGQGTTVTVSS |
| SEQ ID NO: 3265 | VH | D- | EVQLVESGEGLVQPGGSLRLSCAVSGFTFRSYGMSWVRQAPGK |
| | | H.41 | GLEWVALISSGGSYTYYTDSVKGRFTISRDNSKNTLYLQMGSLR |
| | | | AEDMAVYYCSRHGGNFFDYWGQGTTVTVSS |
| SEQ ID NO: 3266 | VH | D- | EVQLVESGGGLVQPGGSLRLSCAVSGFTFRSYGMSWVRQAPGK |
| | | H.42 | GLEWVALISSGGSYTYYTDSVKGRFTISRDNSKNTLYLQMGSLR |
| | | | AEDMAVYYCSRHGGNFFDYWGQGTTVTVSS |
| SEQ ID NO: 3267 | VH | D- | EVQLVESGGGLVQPGGSLRLSCSVSGFTFRSYGMSWVRQAPGK |
| | | H.43 | GLEWVALISSGGSYTYYTDSVKGRFTISRDNSKNTLYVQMSSLR |
| | | | AEDTAVYYCSRHGGNFFDYWGQGTTVTVSS |
| SEQ ID NO: 3268 | VH | D- | EVQLVESGGGLVQPGGSLRLSCAVSGFTFRSYGMSWVRQAPGK |
| | | H.44 | GLEWVALISSGGSYTYYTDSVKGRFIISRDNSRNSLYLQKNRRR |
| | | | AEDMAVYYCSRHGGNFFDYWGQGTTVTVSS |
| SEQ ID NO: 3269 | VH | D- | EVQLVESGGGLVQPGGSLRLSCAVSGFTFRSYGMSWVHQAPGK |
| | | H.45 | GLEWVALISSGGSYTYYTDSVKGRFIISRDNSRNTLYLQTNSLRA |
| | | | EDTAVYYCSRHGGNFFDYWGQGTTVTVSS |
| SEQ ID NO: 3270 | VH | D- | EVHLVESGGGLVQPGGALRLSCAVSGFTFRSYGMSWVRQATGK |
| | | H.46 | GLEWVALISSGGSYTYYTDSVKGRFTISRENAKNSLYLQMNSLR |
| | | | AGDTAVYYCSRHGGNFFDYWGQGTTVTVSS |
| SEQ ID NO: 3271 | VH | D- | EVQLVESGGGLVQPRQSLRLSCAVSGFTFRSYGMSWVRQAPGK |
| | | H.47 | GLEWVALISSGGSYTYYTDSVKGRFTISRDNSKNTLYLQMNNLR |
| | | | AEGTAVYYCSRHGGNFFDYWGQGTTVTVSS |
| SEQ ID NO: 3272 | VH | D- | EVQLVESGGGLVQPRGSLRLSCAVSGFTFRSYGMSWVRQAPGK |
| | | H.48 | GLEWVALISSGGSYTYYTDSVKGRFTISRDNSKNTLYLQMNNLR |
| | | | AEGTAAYYCSRHGGNFFDYWGQGTTVTVSS |
| SEQ ID NO: 3273 | VH | D- | QVQLVQSGAEVKKPGASVKVSCKVSGFTFRSYGMSWVRQAPG |
| | | H.49 | KGLEWVALISSGGSYTYYTDSVKGRFTITRDNSTNTLYMELSSL |
| | | | RSEDTAVYYCSRHGGNFFDYWGQGTTVTVSS |
| SEQ ID NO: 3274 | VH | D- | QVQLVQSGSELKKPGASVKVSCKVSGFTFRSYGMSWVRQAPGQ |
| | | H.50 | GLEWVALISSGGSYTYYTDSVKGRFVISRDNSVNTLYLQISSLKA |
| | | | EDTAVYYCSRHGGNFFDYWGQGTTVTVSS |

TABLE 13
Amino acid sequences for additional anti-TCRβ V antibodies. Amino acid and nucleotide
sequences for murine and humanized antibody molecules which bind to various TCRVB
families are disclosed. The amino acid the heavy and light chain CDRs, and the amino
acid and nucleotide sequences of the heavy and light chain variable regions, and the
heavy and light chains are shown. Antibodies disclosed in the table includeIMMU222.
IMMU 222 binds human TCRβV 6-5, TCRβV 6-6, or TCRβV 6-9 (TCRBβ13.1 per old nomenclature).

IMMU222 (murine) also referred to as BJ1461; or Antibody I
Binds to human TCRVβ 6-5, 6-6, 6-9

| | | |
|---|---|---|
| SEQ ID NO: 1141 | HC CDR1 (Kabat) | SYAMS |
| SEQ ID NO: 1142 | HC CDR2 (Kabat) | HISNGGDYIYYADTVKG |
| SEQ ID NO: 1143 | HC CDR3 (Kabat) | PSYYSDPWFFDV |
| SEQ ID NO: 1144 | HC CDR1 (Chothia) | GFTFRSY |
| SEQ ID NO: 1145 | HC CDR2 (Chothia) | SNGGDY |
| SEQ ID NO: 1143 | HC CDR3 (Chothia) | PSYYSDPWFFDV |
| SEQ ID NO: 1146 | HC CDR1 | GFTFRSYAMS |
| | Combined) | |
| SEQ ID NO: 1142 | HC CDR2 | HISNGGDYIYYADTVKG |
| | (Combined) | |
| SEQ ID NO: 1143 | HC CDR3 (Combined) | PSYYSDPWFFDV |
| SEQ ID NO: 1147 | LC CDR1 (Kabat) | SAGSSVSFMH |
| SEQ ID NO: 1148 | LC CDR2 (Kabat) | DTSKLAS |
| SEQ ID NO: 1149 | LC CDR3 (Kabat) | LQGSGFPLT |
| SEQ ID NO: 1150 | LC CDR1 (Chothia) | GSSVSF |
| SEQ ID NO: 1148 | LC CDR2 (Chothia) | DTSKLAS |
| SEQ ID NO: 1149 | LC CDR3 (Chothia) | LQGSGFPLT |
| SEQ ID NO: 1147 | LC CDR1 (Combined) | SAGSSVSFMH |
| SEQ ID NO: 1148 | LC CDR2 (Combined) | DTSKLAS |
| SEQ ID NO: 1149 | LC CDR3 (Combined) | LQGSGFPLT |
| SEQ ID NO: 1151 | VL | ENVLTQSPAIMSASPGEKVTMTCSAGSSVSFMHWYQQKSSTSP |
| | | KLWIYDTSKLASGVPGRFSGSGSGNSFSLTISSMEAEDVAIYYC |
| | | LQGSGFPLTFGSGTKLEIK |
| SEQ ID NO: 1152 | VH | DVKLVESGEGLVKPGGSLKLSCAASGFTFRSYAMSWVRQTPE |
| | | KRLEWVAHISNGGDYIYYADTVKGRFTISRDNARNTLYLQMS |
| | | SLKSEDTAMYYCTRPSYYSDPWFFDVWGTGTTVTVSS |

VH for IMMU222 (humanized) also referred to as Antibody I-H
Binds to human TCRVβ6-5, 6-6, 6-9

| | | |
|---|---|---|
| SEQ ID NO: 1153 | VH-1 | EVQLVESGGGLVQPGGSLRLSCAASGFTFRSYAMSWVRQAPG |
| | | KGLEWVAHISNGGDYIYYADTVKGRFTISRDNAKNSLYLQMN |
| | | SLRAEDTAVYYCTRPSYYSDPWFFDVWGQGTTVTVSS |
| SEQ ID NO: 1154 | VH-2 | QVQLVESGGGVVQPGRSLRLSCAASGFTFRSYAMSWVRQAPG |
| | | KGLEWVAHISNGGDYIYYADTVKGRFTISRDNSKNTLYLQMS |
| | | SLRAEDTAVYYCTRPSYYSDPWFFDVWGQGTTVTVSS |
| SEQ ID NO: 1155 | VH-3 | EVQLVESGGGLVQPGGSLRLSCAASGFTFRSYAMSWVRQAPG |
| | | KGLEWVAHISNGGDYIYYADTVKGRFTISRDNSKNTLYLQMN |
| | | SLRAEDTAVYYCTRPSYYSDPWFFDVWGQGTTVTVSS |
| SEQ ID NO: 1156 | VH-4 | QVQLVQSGSELKKPGASVKVSCKASGFTFRSYAMSWVRQAPG |
| | | QGLEWVAHISNGGDYIYYADTVKGRFVISRDNSVNTLYLQISS |
| | | LKAEDTAVYYCTRPSYYSDPWFFDVWGQGTTVTVSS |
| SEQ ID NO: 1157 | VH-5 | QVQLVQSGAEVKKPGASVKVSCKASGFTFRSYAMSWVRQAP |
| | | GQRLEWVAHISNGGDYIYYADTVKGRFTITRDNSANTLYMEL |
| | | SSLRSEDTAVYYCTRPSYYSDPWFFDVWGQGTTVTVSS |

VL for IMMU222 (humanized)) also referred to as Antibody I-H
Binds to human TCRVβ6-5, 6-6, 6-9

| | | |
|---|---|---|
| SEQ ID NO: 1158 | VL-1 | ENVLTQSPATLSLSPGERATLSCSAGSSVSFMHWYQQKPGQAP |
| | | KLLIYDTSKLASGIPARFSGSGSQNDFTLTISSLEPEDFAVYYCL |
| | | QGSGFPLTFGQGTKLEIK |
| SEQ ID NO: 1159 | VL-2 | ENVLTQSPDFQSVTPKEKVTITCSAGSSVSFMHWYQQKPDQSP |
| | | KLLIYDTSKLASGVPSRFSGSGSGNDFTLTINSLEAEDAATYYC |
| | | LQGSGFPLTFGQGTKLEIK |
| SEQ ID NO: 1160 | VL-3 | DNQLTQSPSSLSASVGDRVTITCSAGSSVSFMHWYQQKPGKVP |
| | | KLLIYDTSKLASGVPSRFSGSGSGNDFTLTISSLQPEDVATYYC |
| | | LQGSGFPLTFGQGTKLEIK |
| SEQ ID NO: 1161 | VL-4 | ANQLTQSPSSLSASVGDRVTITCSAGSSVSFMHWYQQKPGKAP |
| | | KLLIYDTSKLASGVPSRFSGSGSGNDFTLTISSLQPEDFATYYCL |
| | | QGSGFPLTFGQGTKLEIK |
| SEQ ID NO: 1162 | VL-5 | DNVLTQSPDSLAVSLGERATINCSAGSSVSFMHWYQQKPGQPP |
| | | KLLIYDTSKLASGVPDRFSGSGSGNDFTLTISSLQAEDVAVYYC |
| | | LQGSGFPLTFGQGTKLEIK |

TABLE 14
Exemplary Fc modifications

| Modification or mutation | Altered effector function |
|---|---|
| Leu235Glu | ADCC: |
| Leu234Ala/Leu235Ala (LALA) | ADCC; ADCP; CDC |
| Ser228Pro/Leu235Glu | |
| Leu234Ala/Leu235Ala/Pro329Gly | ADCP |
| Pro331Ser/Leu234Glu/Leu235Phe | CDC |
| Asp265Ala | ADCC, ADCP |
| Gly237Ala | ADCP |
| Glu318Ala | ADCP |
| Glu233Pro | |
| Gly236Arg/Leu328Arg | ADCC |
| His268Gln/Val309Leu/Ala330Ser/Pro331Ser | ADCC; ADCP; CDC |
| Val234Ala/Gly237Ala/Pro238Ser/ | ADCC; ADCP; CDC |
| His268Ala/Val309Leu/Ala330Ser/Pro331Ser | |
| Leu234Ala/L235Ala/Gly237Ala/P238Ser/ | ADCC; CDC |
| His268Ala/Ala330Ser/Pro331Ser | |
| Ala330Leu | CDC |
| Asp270Ala | CDC |
| Lys322Ala | CDC |
| Pro329Ala | CDC |
| Pro331Ala | CDC |
| Val264Ala | CDC |
| High mannose glycosylation | CDC |
| Phe241Ala | CDC |
| Asn297Ala or Gly or Gln | ADCC; ADCP; CDC |
| S228P/Phe234Ala/Leu235Ala | ADCC; CDC |

TABLE 21
Exemplary Construct Sequences
mBKM0186

| | SEQ | | |
|---|---|---|---|
| | ID | | |
| | NO: | | |
| Chain 1 | 3529 | Full Sequence | QVQLQQSGTELMKPGASVKISCKASGYTFSNYWIEWIKQRPGHGL |
| (heavy chain) | | | EWVGEILPGAGPTNYNEKFKGKATFTADSSSNTAYMQLSSLTSED |
| (CDRs | | | SAVYYCARTDYDYDWFAYWGQGTLVTVSAAKTTAPSVYPLAPV |
| underlined) | | | SGDTTGSSVTLGCLVKGYFPEPVTLTWNSGSLSSGVHTFPAVLQSD |
| | | | LYTLSSSVTVTSSTWPSQSITCNVAHPASSTKVDKKIEPKSCDKTHT |
| | | | CPPCPAPNAAGGPSVFIFPPKIKDVLMISLSPIVTCVVVDVSEDDPD |
| | | | VQISWFVNNVEVHTAQTQTHREDYNSTLRVVSALPIQHQDWMSG |
| | | | KEFKCKVNNKDLGAPIERTISKPKGSVRAPQVCVLPPPEEEMTKKQ |
| | | | VTLSCAVTDFMPEDIYVEWTNNGKTELNYKNTEPVLDSDGSYFM |
| | | | VSKLRVEKKNWVERNSYSCSVVHEGLHNHHTTKSFSRTPGKEPEA |
| | 3548 | Full Sequence | QVQLQQSGTELMKPGASVKISCKASGYTFSNYWIEWIKQRPGHGL |
| | | without tag | EWVGEILPGAGPTNYNEKFKGKATFTADSSSNTAYMQLSSLTSED |
| | | | SAVYYCARTDYDYDWFAYWGQGTLVTVSAAKTTAPSVYPLAPV |
| | | | SGDTTGSSVTLGCLVKGYFPEPVTLTWNSGSLSSGVHTFPAVLQSD |
| | | | LYTLSSSVTVTSSTWPSQSITCNVAHPASSTKVDKKIEPKSCDKTHT |
| | | | CPPCPAPNAAGGPSVFIFPPKIKDVLMISLSPIVTCVVVDVSEDDPD |
| | | | VQISWFVNNVEVHTAQTQTHREDYNSTLRVVSALPIQHQDWMSG |
| | | | KEFKCKVNNKDLGAPIERTISKPKGSVRAPQVCVLPPPEEEMTKKQ |
| | | | VTLSCAVTDFMPEDIYVEWTNNGKTELNYKNTEPVLDSDGSYFM |
| | | | VSKLRVEKKNWVERNSYSCSVVHEGLHNHHTTKSFSRTPGK |
| | 3530 | anti-TCRvβ VH | QVQLQQSGTELMKPGASVKISCKASGYTFSNYWIEWIKQRPGHGL |
| | | from murine | EWVGEILPGAGPTNYNEKFKGKATFTADSSSNTAYMQLSSLTSED |
| | | MR5-2 | SAVYYCARTDYDYDWFAYWGQGTLVTVSAAKTTAPSVYPLAPV |
| | | | SGDTTGSSVTLGCLVKGYFPEPVTLTWNSGSLSSGVHTFPAVLQSD |
| | | | LYTLSSSVTVTSS |
| | 3531 | mouse Fc heavy | PSVFIFPPKIKDVLMISLSPIVTCVVVDVSEDDPDVQISWFVNNVEV |
| | | chain comprising | TWPSQSITCNVAHPASSTKVDKKIEPKSCDKTHTCPPCPAPNAAGG |
| | | LALAPG mutations | HTAQTQTHREDYNSTLRVVSALPIQHQDWMSGKEFKCKVNNKDL |
| | | (L234A L235A | GAPIERTISKPKGSVRAPQVCVLPPPEEEMTKKQVTLSCAVTDFMP |
| | | and P329G) | EDIYVEWTNNGKTELNYKNTEPVLDSDGSYFMVSKLRVEKKNWV |
| | | | ERNSYSCSVVHEGLHNHHTTKSFSRTPGK |
| | 3547 | Tag | EPEA |
| Chain 2 | 3532 | Full Sequence | APTSSSTKKTQLQLEHLLLDLQMILNGINNYKNPKLTRMLTFKFY |
| (IL2-Fc) | | | MPKKATELKHLQCLEEELKPLEEVLNLAQSKNFHLRPRDLISNINV |
| | | | IVLELKGSETTFMCEYADETATIVEFLNRWITFCQSIISTLTGGGGS |
| | | | GGGGSDKTHTCPPCPAPNAAGGPSVFIFPPKIKDVLMISLSPIVTCV |
| | | | VVDVSEDDPDVQISWFVNNVEVHTAQTQTHREDYNSTLRVVSAL |
| | | | PIQHQDWMSGKEFKCKVNNKDLGAPIERTISKPKGSVRAPQVYVL |
| | | | PPCEEEMTKKQVTLWCMVTDFMPEDIYVEWTNNGKTELNYKNTE |
| | | | PVLDSDGSYFMYSKLRVEKKNWVERNSYSCSVVHEGLHNHHTTK |
| | | | SFSRTPGKGGGGSHHHHHHHH |
| | 3549 | Full Sequence | APTSSSTKKTQLQLEHLLLDLQMILNGINNYKNPKLTRMLTFKFY |
| | | without tag | MPKKATELKHLQCLEEELKPLEEVLNLAQSKNFHLRPRDLISNINV |
| | | | IVLELKGSETTFMCEYADETATIVEFLNRWITFCQSIISTLTGGGGS |
| | | | GGGGSDKTHTCPPCPAPNAAGGPSVFIFPPKIKDVLMISLSPIVTCV |
| | | | VVDVSEDDPDVQISWFVNNVEVHTAQTQTHREDYNSTLRVVSAL |
| | | | PIQHQDWMSGKEFKCKVNNKDLGAPIERTISKPKGSVRAPQVYVL |
| | | | PPCEEEMTKKQVTLWCMVTDFMPEDIYVEWTNNGKTELNYKNTE |
| | | | PVLDSDGSYFMYSKLRVEKKNWVERNSYSCSVVHEGLHNHHTTK |
| | | | SFSRTPGK |
| | 2191 | IL-2 | APTSSSTKKTQLQLEHLLLDLQMILNGINNYKNPKLTRMLTFKFY |
| | | | MPKKATELKHLQCLEEELKPLEEVLNLAQSKNFHLRPRDLISNINV |
| | | | IVLELKGSETTFMCEYADETATIVEFLNRWITFCQSIISTLT |
| | 3308 | linker | GGGGSGGGGS |
| | 3533 | mouse Fc heavy | EDDPDVQISWFVNNVEVHTAQTQTHREDYNSTLRVVSALPIQHQD |
| | | chain comprising | DKTHTCPPCPAPNAAGGPSVFIFPPKIKDVLMISLSPIVTCVVVDVS |
| | | LALAPG mutations | WMSGKEFKCKVNNKDLGAPIERTISKPKGSVRAPQVYVLPPCEEE |
| | | (L234A L235A | MTKKQVTLWCMVTDFMPEDIYVEWTNNGKTELNYKNTEPVLDS |
| | | and P329G) | DGSYFMYSKLRVEKKNWVERNSYSCSVVHEGLHNHHTTKSFSRT |
| | | | PGK |
| | 3534 | Tag | GGGGSHHHHHHHH |
| Chain 3 | 3526 | Full Sequence | DIVMSQSPSSLAVSVGEKVTMSCKSSQSLLYSGNQKNYLAWYQQ |
| (light chain) | | | KPGQSPKLLIYWASTRESGVPDRFTGSGSGTDFTLTINSVKAEDLT |
| (CDRs | | | VYYCQQYYGYPRTFGGGTKVEIKRADAAPTVSIFPPSSEQLTSGGA |
| underlined) | | | SVVCFLNNFYPKDINVKWKIDGSERQNGVLNSWTDQDSKDSTYS |
| | | | MSSTLTLTKDEYERHNSYTCEATHKTSTSPIVKSFNRNEC |
| | 3527 | anti-TCRVβVL | DIVMSQSPSSLAVSVGEKVTMSCKSSQSLLYSGNQKNYLAWYQQ |
| | | from murine | KPGQSPKLLIYWASTRESGVPDRFTGSGSGTDFTLTINSVKAEDLT |
| | | MR5-2 | VYYCQQYYGYPRTFGGGTKVEIK |
| | 3528 | Immunoglobulin | RADAAPTVSIFPPSSEQLTSGGASVVCFLNNFYPKDINVKWKIDGS |
| | | constant region | ERQNGVLNSWTDQDSKDSTYSMSSTLTLTKDEYERHNSYTCEAT |
| | | | HKTSTSPIVKSFNRNEC |

mBKM0281

| | SEQ | | |
|---|---|---|---|
| | ID | | |
| | NO: | | |
| Chain 1 | 3536 | Full Sequence | QVQLQQSGTELMKPGASVKISCKASGYTFSNYWIEWIKQRPGHGL |
| (heavy chain) | | | EWVGEILPGAGPTNYNEKFKGKATFTADSSSNTAYMQLSSLTSED |
| (CDRs | | | SAVYYCARTDYDYDWFAYWGQGTLVTVSAAKTTAPSVYPLAPV |
| underlined) | | | SGDTTGSSVTLGCLVKGYFPEPVTLTWNSGSLSSGVHTFPAVLQSD |
| | | | LYTLSSSVTVTSSTWPSQSITCNVAHPASSTKVDKKIEPKSCDKTHT |
| | | | CPPCPAPNAAGGPSVFIFPPKIKDVLMISLSPIVTCVVVDVSEDDPD |
| | | | VQISWFVNNVEVHTAQTQTHREDYNSTLRVVSALPIQHQDWMSG |
| | | | KEFKCKVNNKDLGAPIERTISKPKGSVRAPQVYVLPPPEEEMTKK |
| | | | QVTLTCMVTDFMPEDIYVEWTNNGKTELNYKNTEPVLDSDGSYF |
| | | | MYSKLRVEKKNWVERNSYSCSVVHEGLHNHHTTKSFSRTPGKEP |
| | | | EA |
| | 3530 | anti-TCRvβ VH | QVQLQQSGTELMKPGASVKISCKASGYTFSNYWIEWIKQRPGHGL |
| | | from murine | EWVGEILPGAGPTNYNEKFKGKATFTADSSSNTAYMQLSSLTSED |
| | | MR5-2 | SAVYYCARTDYDYDWFAYWGQGTLVTVSAAKTTAPSVYPLAPV |
| | | | SGDTTGSSVTLGCLVKGYFPEPVTLTWNSGSLSSGVHTFPAVLQSD |
| | | | LYTLSSSVTVTSS |
| | 3537 | mouse Fc heavy | PSVFIFPPKIKDVLMISLSPIVTCVVVDVSEDDPDVQISWFVNNVEV |
| | | chain comprising | TWPSQSITCNVAHPASSTKVDKKIEPKSCDKTHTCPPCPAPNAAGG |
| | | LALAPG mutations | HTAQTQTHREDYNSTLRVVSALPIQHQDWMSGKEFKCKVNNKDL |
| | | (L234A L235A | GAPIERTISKPKGSVRAPQVYVLPPPEEEMTKKQVTLTCMVTDFMP |
| | | and P329G) | EDIYVEWTNNGKTELNYKNTEPVLDSDGSYFMYSKLRVEKKNWV |
| | | | ERNSYSCSVVHEGLHNHHTTKSFSRTPGKEPEA |
| Chain 2 | 3535 | Full Sequence | DIVMSQSPSSLAVSVGEKVTMSCKSSQSLLYSGNQKNYLAWYQQ |
| (light chain- | | | KPGQSPKLLIYWASTRESGVPDRFTGSGSGTDFTLTINSVKAEDLT |
| IL2) | | | VYYCQQYYGYPRTFGGGTKVEIKRADAAPTVSIFPPSSEQLTSGGA |
| (CDRs | | | SVVCFLNNFYPKDINVKWKIDGSERQNGVLNSWTDQDSKDSTYS |
| underlined) | | | MSSTLTLTKDEYERHNSYTCEATHKTSTSPIVKSFNRNECGGGGSG |
| | | | GGGSGGGGSAPTSSSTKKTQLQLEHLLLDLQMILNGINNYKNPKL |
| | | | TRMLTFKFYMPKKATELKHLQCLEEELKPLEEVLNLAQSKNFHLR |
| | | | PRDLISNINVIVLELKGSETTFMCEYADETATIVEFLNRWITFCQ |
| | | | SIISTLT |
| | 3527 | anti-TCRVβVL | DIVMSQSPSSLAVSVGEKVTMSCKSSQSLLYSGNQKNYLAWYQQ |
| | | from murine | KPGQSPKLLIYWASTRESGVPDRFTGSGSGTDFTLTINSVKAEDLT |
| | | MR5-2 | VYYCQQYYGYPRTFGGGTKVEIK |
| | 3528 | Immunoglobulin | RADAAPTVSIFPPSSEQLTSGGASVVCFLNNFYPKDINVKWKIDGS |
| | | constant region | ERQNGVLNSWTDQDSKDSTYSMSSTLTLTKDEYERHNSYTCEATHK |
| | | | TSTSPIVKSFNRNEC |
| | 3309 | linker | GGGGSGGGGSGGGGS |
| | 2191 | IL-2 | APTSSSTKKTQLQLEHLLLDLQMILNGINNYKNPKLTRMLTFKFY |
| | | | MPKKATELKHLQCLEEELKPLEEVLNLAQSKNFHLRPRDLISNINV |
| | | | IVLELKGSETTFMCEYADETATIVEFLNRWITFCQSIISTLT |

Humanized Molecule 1

| | SEQ | | |
|---|---|---|---|
| | ID | | |
| | NO: | | |
| Chain 1 | 3517 | Full Sequence | QVQLVQSGAEVKKPGSSVKVSCKASGHDFRLTYIHWVRQAPGQG |
| (heavy chain) | | | LEWMGRVSAGSGNVKYNEKFKGRVTITADTSTSTAYMELSSLRSE |
| (CDRs | | | DTAVYYCAVSYYSYDVLDYWGQGTTVTVSSASTKGPSVFPLAPSS |
| underlined) | | | KSTSGGTAALGCLVKDYFPEPVTVSWNSGALTSGVHTFPAVLQSS |
| | | | GLYSLSSVVTVPSSSLGTQTYICNVNHKPSNTKVDKRVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYASTYRVVSVLTVLHQD |
| | | | WLNGKEYKCKVSNKALPAPIEKTISKAKGQPREPQVCTLPPSREE |
| | | | MTKNQVSLSCAVKGFYPSDIAVEWESNGQPENNYKTTPPVLDSDG |
| | | | SFFLVSKLTVDKSRWQQGNVFSCSVMHEALHNHYTQKSLSLSPGK |
| | 1346 | anti-TCRVβVH | QVQLVQSGAEVKKPGSSVKVSCKASGHDFRLTYIHWVRQAPGQG |
| | | | LEWMGRVSAGSGNVKYNEKFKGRVTITADTSTSTAYMELSSLRSE |
| | | | DTAVYYCAVSYYSYDVLDYWGQGTTVTVSS |
| | 3649 | Human IgG1 | ASTKGPSVFPLAPSSKSTSGGTAALGCLVKDYFPEPVTVSWNSGAL |
| | | hole cys N297A | TSGVHTFPAVLQSSGLYSLSSVVTVPSSSLGTQTYICNVNHKPSNT |
| | | | KVDKRVEPKSCDKTHTCPPCPAPELLGGPSVFLFPPKPKDTLMISR |
| | | | TPEVTCVVVDVSHEDPEVKFNWYVDGVEVHNAKTKPREEQYAST |
| | | | YRVVSVLTVLHQDWLNGKEYKCKVSNKALPAPIEKTISKAKGQPR |
| | | | EPQVCTLPPSREEMTKNQVSLSCAVKGFYPSDIAVEWESNGQPEN |
| | | | NYKTTPPVLDSDGSFFLVSKLTVDKSRWQQGNVFSCSVMHEALH |
| | | | NHYTQKSLSLSPGK |
| Chain 2 | 3521 | Full Sequence | APTSSSTKKTQLQLEHLLLDLQMILNGINNYKNPKLTRMLTFKFY |
| (IL2-Fc) | | | MPKKATELKHLQCLEEELKPLEEVLNLAQSKNFHLRPRDLISNINV |
| | | | IVLELKGSETTFMCEYADETATIVEFLNRWITFAQSIISTLTGGGGS |
| | | | GGGGSDKTHTCPPCPAPELLGGPSVFLFPPKPKDTLMISRTPEVTC |
| | | | VVVDVSHEDPEVKFNWYVDGVEVHNAKTKPREEQYASTYRVVS |
| | | | VLTVLHQDWLNGKEYKCKVSNKALPAPIEKTISKAKGQPREPQVY |
| | | | TLPPCREEMTKNQVSLWCLVKGFYPSDIAVEWESNGQPENNYKTT |
| | | | PPVLDSDGSFFLYSKLTVDKSRWQQGNVFSCSVMHEALHNHYTQ |
| | | | KSLSLSPGK |
| | 2270 | JIL-2 C125A | APTSSSTKKTQLQLEHLLLDLQMILNGINNYKNPKLTRMLTFKFY |
| | | | MPKKATELKHLQCLEEELKPLEEVLNLAQSKNFHLRPRDLISNINV |
| | | | IVLELKGSETTFMCEYADETATIVEFLNRWITFAQSIISTLT |
| | 3308 | linker | GGGGSGGGGS |
| | 3648 | Human Fc Knob | DKTHTCPPCPAPELLGGPSVFLFPPKPKDTLMISRTPEVTCVVVDV |
| | | cys N297A | SHEDPEVKFNWYVDGVEVHNAKTKPREEQYASTYRVVSVLTVLH |
| | | | QDWLNGKEYKCKVSNKALPAPIEKTISKAKGQPREPQVYTLPPCR |
| | | | EEMTKNQVSLWCLVKGFYPSDIAVEWESNGQPENNYKTTPPVLD |
| | | | SDGSFFLYSKLTVDKSRWQQGNVFSCSVMHEALHNHYTQKSLSLS |
| | | | PGK |
| Chain 3 | 3518 | Full Sequence | DIQMTQSPSFLSASVGDRVTITCKASQNVADRVVWHQQKPGKAP |
| (light chain) | | | KALIYSSSHRYKGVPSRFSGSGSGTEFTLTISSLQPEDFATYFCQQF |
| (CDRs | | | KSYPLTFGQGTKLEIKRTVAAPSVFIFPPSDEQLKSGTASVVCLLNN |
| underlined) | | | FYPREAKVQWKVDNALQSGNSQESVTEQDSKDSTYSLSSTLTLSK |
| | | | ADYEKHKVYACEVTHQGLSSPVTKSFNRGEC |
| | 1349 | anti-TCRvβ VL | DIQMTQSPSFLSASVGDRVTITCKASQNVADRVVWHQQKPGKAP |
| | | | KALIYSSSHRYKGVPSRFSGSGSGTEFTLTISSLQPEDFATYFCQQF |
| | | | KSYPLTFGQGTKLEIK |
| | 3644 | Immunoglobulin | RTVAAPSVFIFPPSDEQLKSGTASVVCLLNNFYPREAKVQWKVDN |
| | | kappa constant C | ALQSGNSQESVTEQDSKDSTYSLSSTLTLSKADYEKHKVYACEVT |
| | | region | HQGLSSPVTKSFNRGEC |

Humanized Molecule 2

| | SEQ | | |
|---|---|---|---|
| | ID | | |
| | NO: | | |
| Chain 1 | 4000 | Full Sequence | QVQLVQSGAEVKKPGSSVKVSCKASGHDFRLTYIHWVRQAPGQG |
| (heavy chain) | | | LEWMGRVSAGSGNVKYNEKFKGRVTITADTSTSTAYMELSSLRSE |
| (CDRs | | | DTAVYYCAVSYYSYDVLDYWGQGTTVTVSSASTKGPSVFPLAPSS |
| underlined) | | | KSTSGGTAALGCLVKDYFPEPVTVSWNSGALTSGVHTFPAVLQSS |
| | | | GLYSLSSVVTVPSSSLGTQTYICNVNHKPSNTKVDKRVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYASTYRVVSVLTVLHQD |
| | | | WLNGKEYKCKVSNKALPAPIEKTISKAKGQPREPQVCTLPPSREE |
| | | | MTKNQVSLSCAVKGFYPSDIAVEWESNGQPENNYKTTPPVLDSDG |
| | | | SFFLVSKLTVDKSRWQQGNVFSCSVMHEALHNHYTQKSLSLSP |
| | 1346 | anti-TCRvβ VH | QVQLVQSGAEVKKPGSSVKVSCKASGHDFRLTYIHWVRQAPGQG |
| | | | LEWMGRVSAGSGNVKYNEKFKGRVTITADTSTSTAYMELSSLRSE |
| | | | DTAVYYCAVSYYSYDVLDYWGQGTTVTVSS |
| | 4001 | Human IgG1 | ASTKGPSVFPLAPSSKSTSGGTAALGCLVKDYFPEPVTVSWNSGAL |
| | | hole cys N297A | TSGVHTFPAVLQSSGLYSLSSVVTVPSSSLGTQTYICNVNHKPSNT |
| | | | KVDKRVEPKSCDKTHTCPPCPAPELLGGPSVFLFPPKPKDTLMISR |
| | | | TPEVTCVVVDVSHEDPEVKFNWYVDGVEVHNAKTKPREEQYAST |
| | | | YRVVSVLTVLHQDWLNGKEYKCKVSNKALPAPIEKTISKAKGQPR |
| | | | EPQVCTLPPSREEMTKNQVSLSCAVKGFYPSDIAVEWESNGQPEN |
| | | | NYKTTPPVLDSDGSFFLVSKLTVDKSRWQQGNVFSCSVMHEALH |
| | | | NHYTQKSLSLSP |
| Chain 2 | 4002 | Full Sequence | APTSSSTKKTQLQLEHLLLDLQMILNGINNYKNPKLTRMLTFKFY |
| (IL2-Fc) | | | MPKKATELKHLQCLEEELKPLEEVLNLAQSKNFHLRPRDLISNINV |
| | | | IVLELKGSETTFMCEYADETATIVEFLNRWITFAQSIISTLTGGGGS |
| | | | GGGGSDKTHTCPPCPAPELLGGPSVFLFPPKPKDTLMISRTPEVTC |
| | | | VVVDVSHEDPEVKFNWYVDGVEVHNAKTKPREEQYASTYRVVS |
| | | | VLTVLHQDWLNGKEYKCKVSNKALPAPIEKTISKAKGQPREPQVY |
| | | | TLPPCREEMTKNQVSLWCLVKGFYPSDIAVEWESNGQPENNYKTT |
| | | | PPVLDSDGSFFLYSKLTVDKSRWQQGNVFSCSVMHEALHNHYTQ |
| | | | KSLSLSP |
| | 2270 | IL-2 C125A | APTSSSTKKTQLQLEHLLLDLQMILNGINNYKNPKLTRMLTFKFY |
| | | | MPKKATELKHLQCLEEELKPLEEVLNLAQSKNFHLRPRDLISNINV |
| | | | IVLELKGSETTFMCEYADETATIVEFLNRWITFAQSIISTLT |
| | 3308 | linker | GGGGSGGGGS |
| | 4031 | Human Fc Knob | IDKTHTCPPCPAPELLGGPSVFLFPPKPKDTLMISRTPEVTCVVVDV |
| | | cys N297A | SHEDPEVKFNWYVDGVEVHNAKTKPREEQYASTYRVVSVLTVLH |
| | | | QDWLNGKEYKCKVSNKALPAPIEKTISKAKGQPREPQVYTLPPCR |
| | | | EEMTKNQVSLWCLVKGFYPSDIAVEWESNGQPENNYKTTPPVLD |
| | | | SDGSFFLYSKLTVDKSRWQQGNVFSCSVMHEALHNHYTQKSLSLS |
| | | | D |
| Chain 3 | 3518 | Full Sequence | DIQMTQSPSFLSASVGDRVTITCKASQNVADRVVWHQQKPGKAP |
| (light chain) | | | KALIYSSSHRYKGVPSRFSGSGSGTEFTLTISSLQPEDFATYFCQQF |
| (CDRs | | | KSYPLTFGQGTKLEIKRTVAAPSVFIFPPSDEQLKSGTASVVCLLNN |
| underlined) | | | FYPREAKVQWKVDNALQSGNSQESVTEQDSKDSTYSLSSTLTLSK |
| | | | ADYEKHKVYACEVTHQGLSSPVTKSFNRGEC |
| | 1349 | anti-TCRvβ VL | DIQMTQSPSFLSASVGDRVTITCKASQNVADRVVWHQQKPGKAP |
| | | | KALIYSSSHRYKGVPSRFSGSGSGTEFTLTISSLQPEDFATYFCQQF |
| | | | KSYPLTFGQGTKLEIK |
| | 3644 | Immunoglobulin | RTVAAPSVFIFPPSDEQLKSGTASVVCLLNNFYPREAKVQWKVDN |
| | | kappa constant | ALQSGNSQESVTEQDSKDSTYSLSSTLTLSKADYEKHKVYACEVT |
| | | C region | HQGLSSPVTKSFNRGEC |

Humanized Molecule 3

| | SEQ | | |
|---|---|---|---|
| | ID | | |
| | NO: | | |
| Chain 1 | 4004 | Full Sequence | QVQLVQSGAEVKKPGSSVKVSCKASGYSFTTYYIHWVRQAPGQG |
| (heavy chain) | | | LEWMGWFFPGSGNIKYNEKFKGRVTITADTSTSTAYMELSSLRSE |
| (CDRs | | | DTAVYYCAGSYYSYDVLDYWGQGTTVTVSSASTKGPSVFPLAPSS |
| underlined) | | | KSTSGGTAALGCLVKDYFPEPVTVSWNSGALTSGVHTFPAVLQSS |
| | | | GLYSLSSVVTVPSSSLGTQTYICNVNHKPSNTKVDKRVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYASTYRVVSVLTVLHQD |
| | | | WLNGKEYKCKVSNKALPAPIEKTISKAKGQPREPQVCTLPPSREE |
| | | | MTKNQVSLSCAVKGFYPSDIAVEWESNGQPENNYKTTPPVLDSDG |
| | | | SFFLVSKLTVDKSRWQQGNVFSCSVMHEALHNHYTQKSLSLSPGK |
| | 9 | anti-TCRvβ VH | QVQLVQSGAEVKKPGSSVKVSCKASGYSFTTYYIHWVRQAPGQG |
| | | | LEWMGWFFPGSGNIKYNEKFKGRVTITADTSTSTAYMELSSLRSE |
| | | | DTAVYYCAGSYYSYDVLDYWGQGTTVTVSS |
| | 3649 | Human IgG1 | ASTKGPSVFPLAPSSKSTSGGTAALGCLVKDYFPEPVTVSWNSGAL |
| | | hole cys N297A | TSGVHTFPAVLQSSGLYSLSSVVTVPSSSLGTQTYICNVNHKPSNT |
| | | | KVDKRVEPKSCDKTHTCPPCPAPELLGGPSVFLFPPKPKDTLMISR |
| | | | TPEVTCVVVDVSHEDPEVKFNWYVDGVEVHNAKTKPREEQYAST |
| | | | YRVVSVLTVLHQDWLNGKEYKCKVSNKALPAPIEKTISKAKGQPR |
| | | | EPQVCTLPPSREEMTKNQVSLSCAVKGFYPSDIAVEWESNGQPEN |
| | | | NYKTTPPVLDSDGSFFLVSKLTVDKSRWQQGNVFSCSVMHEALH |
| | | | NHYTQKSLSLSPGK |
| Chain 2 | 4030 | Full Sequence | APASSSTKKTQLQLEHLLLDLQMILNGINNYKNPKLTRMLTAKFA |
| (IL2-Fc) | | | MPKKATELKHLQCLEEELKPLEEVLNGAQSKNFHLRPRDLISNINV |
| | | | IVLELKGSETTFMCEYADETATIVEFLNRWITFAQSIISTLTGGGGS |
| | | | GGGGSDKTHTCPPCPAPELLGGPSVFLFPPKPKDTLMISRTPEVTC |
| | | | VVVDVSHEDPEVKFNWYVDGVEVHNAKTKPREEQYASTYRVVS |
| | | | VLTVLHQDWLNGKEYKCKVSNKALPAPIEKTISKAKGQPREPQVY |
| | | | TLPPCREEMTKNQVSLWCLVKGFYPSDIAVEWESNGQPENNYKTT |
| | | | PPVLDSDGSFFLYSKLTVDKSRWQQGNVFSCSVMHEALHNHYTQ |
| | | | KSLSLSPGK |
| | 2280 | IL-2 | APASSSTKKTQLQLEHLLLDLQMILNGINNYKNPKLTRMLTAKFA |
| | | | MPKKATELKHLQCLEEELKPLEEVLNGAQSKNFHLRPRDLISNINV |
| | | | IVLELKGSETTFMCEYADETATIVEFLNRWITFAQSIISTLT |
| | 3308 | linker | GGGGSGGGGS |
| | 3648 | Human Fc Knob | DKTHTCPPCPAPELLGGPSVFLFPPKPKDTLMISRTPEVTCVVVDV |
| | | cys N297A | SHEDPEVKFNWYVDGVEVHNAKTKPREEQYASTYRVVSVLTVLH |
| | | | QDWLNGKEYKCKVSNKALPAPIEKTISKAKGQPREPQVYTLPPCR |
| | | | EEMTKNQVSLWCLVKGFYPSDIAVEWESNGQPENNYKTTPPVLD |
| | | | SDGSFFLYSKLTVDKSRWQQGNVFSCSVMHEALHNHYTQKSLSLS |
| | | | PGK |
| Chain 3 | 4005 | Full Sequence | DIQMTQSPSSLSASVGDRVTITCKASQNVGINVVWHQQKPGKVPK |
| (light chain) | | | ALIYSSSHRYSGVPSRFSGSGSGTDFTLTISSLQPEDVATYFCQQFK |
| (CDRs | | | SYPLTFGQGTKLEIKRTVAAPSVFIFPPSDEQLKSGTASVVCLLNNF |
| underlined) | | | YPREAKVQWKVDNALQSGNSQESVTEQDSKDSTYSLSSTLTLSKA |
| | | | DYEKHKVYACEVTHQGLSSPVTKSFNRGEC |
| | | anti-TCRvβ VL | DIQMTQSPSSLSASVGDRVTITCKASQNVGINVVWHQQKPGKVPK |
| | | | ALIYSSSHRYSGVPSRFSGSGSGTDFTLTISSLQPEDVATYFCQQFK |
| | | | SYPLTFGQGTKLEIK |
| | 3644 | Immunoglobulin | RTVAAPSVFIFPPSDEQLKSGTASVVCLLNNFYPREAKVQWKVDN |
| | | kappa constant C | ALQSGNSQESVTEQDSKDSTYSLSSTLTLSKADYEKHKVYACEVT |
| | | region | HQGLSSPVTKSFNRGEC |

Humanized Molecule 4

| | SEQ | | |
|---|---|---|---|
| | ID | | |
| | NO: | | |
| Chain 1 | 4006 | Full Sequence | QVQLVQSGAEVKKPGSSVKVSCKASGYSFTTYYIHWVRQAPGQG |
| (heavy chain) | | | LEWMGWFFPGSGNIKYNEKFKGRVTITADTSTSTAYMELSSLRSE |
| (CDRs | | | DTAVYYCAGSYYSYDVLDYWGQGTTVTVSSASTKGPSVFPLAPSS |
| underlined) | | | KSTSGGTAALGCLVKDYFPEPVTVSWNSGALTSGVHTFPAVLQSS |
| | | | GLYSLSSVVTVPSSSLGTQTYICNVNHKPSNTKVDKRVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYASTYRVVSVLTVLHQD |
| | | | WLNGKEYKCKVSNKALPAPIEKTISKAKGQPREPQVCTLPPSREE |
| | | | MTKNQVSLSCAVKGFYPSDIAVEWESNGQPENNYKTTPPVLDSDG |
| | | | SFFLVSKLTVDKSRWQQGNVFSCSVMHEALHNHYTQKSLSLSP |
| | 9 | anti-TCRvβ VH | QVQLVQSGAEVKKPGSSVKVSCKASGYSFTTYYIHWVRQAPGQG |
| | | | LEWMGWFFPGSGNIKYNEKFKGRVTITADTSTSTAYMELSSLRSE |
| | | | DTAVYYCAGSYYSYDVLDYWGQGTTVTVSS |
| | 4001 | Human IgG1 | ASTKGPSVFPLAPSSKSTSGGTAALGCLVKDYFPEPVTVSWNSGAL |
| | | hole cys N297A | TSGVHTFPAVLQSSGLYSLSSVVTVPSSSLGTQTYICNVNHKPSNT |
| | | | KVDKRVEPKSCDKTHTCPPCPAPELLGGPSVFLFPPKPKDTLMISR |
| | | | TPEVTCVVVDVSHEDPEVKFNWYVDGVEVHNAKTKPREEQYAST |
| | | | YRVVSVLTVLHQDWLNGKEYKCKVSNKALPAPIEKTISKAKGQPR |
| | | | EPQVCTLPPSREEMTKNQVSLSCAVKGFYPSDIAVEWESNGQPEN |
| | | | NYKTTPPVLDSDGSFFLVSKLTVDKSRWQQGNVFSCSVMHEALH |
| | | | NHYTQKSLSLSP |
| Chain 2 | 4007 | Full Sequence | APASSSTKKTQLQLEHLLLDLQMILNGINNYKNPKLTRMLTAKFA |
| (IL2-Fc) | | | MPKKATELKHLQCLEEELKPLEEVLNGAQSKNFHLRPRDLISNINV |
| | | | IVLELKGSETTFMCEYADETATIVEFLNRWITFAQSIISTLTGGGGS |
| | | | GGGGSDKTHTCPPCPAPELLGGPSVFLFPPKPKDTLMISRTPEVTC |
| | | | VVVDVSHEDPEVKFNWYVDGVEVHNAKTKPREEQYASTYRVVS |
| | | | VLTVLHQDWLNGKEYKCKVSNKALPAPIEKTISKAKGQPREPQVY |
| | | | TLPPCREEMTKNQVSLWCLVKGFYPSDIAVEWESNGQPENNYKTT |
| | | | PPVLDSDGSFFLYSKLTVDKSRWQQGNVFSCSVMHEALHNHYTQ |
| | | | KSLSLSP |
| | 2280 | IL-2 | APASSSTKKTQLQLEHLLLDLQMILNGINNYKNPKLTRMLTAKFA |
| | | | MPKKATELKHLQCLEEELKPLEEVLNGAQSKNFHLRPRDLISNINV |
| | | | IVLELKGSETTFMCEYADETATIVEFLNRWITFAQSIISTLT |
| | 3308 | linker | GGGGSGGGGS |
| | 4031 | Human Fc Knob | DKTHTCPPCPAPELLGGPSVFLFPPKPKDTLMISRTPEVTCVVVDV |
| | | cys N297A | SHEDPEVKFNWYVDGVEVHNAKTKPREEQYASTYRVVSVLTVLH |
| | | | QDWLNGKEYKCKVSNKALPAPIEKTISKAKGQPREPQVYTLPPCR |
| | | | EEMTKNQVSLWCLVKGFYPSDIAVEWESNGQPENNYKTTPPVLD |
| | | | SDGSFFLYSKLTVDKSRWQQGNVFSCSVMHEALHNHYTQKSLSLS |
| | | | P |
| Chain 3 | 4005 | Full Sequence | DIQMTQSPSSLSASVGDRVTITCKASQNVGINVVWHQQKPGKVPK |
| (light chain) | | | ALIYSSSHRYSGVPSRFSGSGSGTDFTLTISSLQPEDVATYFCQQFK |
| (CDRs | | | SYPLTFGQGTKLEIKRTVAAPSVFIFPPSDEQLKSGTASVVCLLNNF |
| underlined) | | | YPREAKVQWKVDNALQSGNSQESVTEQDSKDSTYSLSSTLTLSKA |
| | | | DYEKHKVYACEVTHQGLSSPVTKSFNRGEC |
| | 11 | anti-TCRvβ VL | DIQMTQSPSSLSASVGDRVTITCKASQNVGINVVWHQQKPGKVPK |
| | | | ALIYSSSHRYSGVPSRFSGSGSGTDFTLTISSLQPEDVATYFCQQFK |
| | | | SYPLTFGQGTKLEIK |
| | 3644 | Immunoglobulin | RTVAAPSVFIFPPSDEQLKSGTASVVCLLNNFYPREAKVQWKVDN |
| | | kappa constant C | ALQSGNSQESVTEQDSKDSTYSLSSTLTLSKADYEKHKVYACEVT |
| | | region | HQGLSSPVTKSFNRGEC |

Humanized Molecule 5

| | SEQ | | |
|---|---|---|---|
| | ID | | |
| | NO: | | |
| Chain 1 | 4008 | Full Sequence | QVQLVQSGAEVKKPGSSVKVSCKASGTDFDKIYIHWVRQAPGQG |
| (heavy chain) | | | LEWMGRVSAGSGNTKYNEKFKGRVTITADTSTSTAYMELSSLRSE |
| (CDRs | | | DTAVYYCAGSYYSYDVLDYWGQGTTVTVSSASTKGPSVFPLAPSS |
| underlined) | | | KSTSGGTAALGCLVKDYFPEPVTVSWNSGALTSGVHTFPAVLQSS |
| | | | GLYSLSSVVTVPSSSLGTQTYICNVNHKPSNTKVDKRVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYASTYRVVSVLTVLHQD |
| | | | WLNGKEYKCKVSNKALPAPIEKTISKAKGQPREPQVCTLPPSREE |
| | | | MTKNQVSLSCAVKGFYPSDIAVEWESNGQPENNYKTTPPVLDSDG |
| | | | SFFLVSKLTVDKSRWQQGNVFSCSVMHEALHNHYTQKSLSLSPGK |
| | 109 | anti-TCRvβ VH | QVQLVQSGAEVKKPGSSVKVSCKASGTDFDKIYIHWVRQAPGQG |
| | | | LEWMGRVSAGSGNTKYNEKFKGRVTITADTSTSTAYMELSSLRSE |
| | | | DTAVYYCAGSYYSYDVLDYWGQGTTVTVSS |
| | 3649 | Human IgG1 | ASTKGPSVFPLAPSSKSTSGGTAALGCLVKDYFPEPVTVSWNSGAL |
| | | hole cys N297A | TSGVHTFPAVLQSSGLYSLSSVVTVPSSSLGTQTYICNVNHKPSNT |
| | | | KVDKRVEPKSCDKTHTCPPCPAPELLGGPSVFLFPPKPKDTLMISR |
| | | | TPEVTCVVVDVSHEDPEVKFNWYVDGVEVHNAKTKPREEQYAST |
| | | | YRVVSVLTVLHQDWLNGKEYKCKVSNKALPAPIEKTISKAKGQPR |
| | | | EPQVCTLPPSREEMTKNQVSLSCAVKGFYPSDIAVEWESNGQPEN |
| | | | NYKTTPPVLDSDGSFFLVSKLTVDKSRWQQGNVFSCSVMHEALH |
| | | | NHYTQKSLSLSPGK |
| Chain 2 | 3521 | Full Sequence | APTSSSTKKTQLQLEHLLLDLQMILNGINNYKNPKLTRMLTFKFY |
| (IL2-Fc) | | | MPKKATELKHLQCLEEELKPLEEVLNLAQSKNFHLRPRDLISNINV |
| | | | IVLELKGSETTFMCEYADETATIVEFLNRWITFAQSIISTLTGGGGS |
| | | | GGGGSDKTHTCPPCPAPELLGGPSVFLFPPKPKDTLMISRTPEVTC |
| | | | VVVDVSHEDPEVKFNWYVDGVEVHNAKTKPREEQYASTYRVVS |
| | | | VLTVLHQDWLNGKEYKCKVSNKALPAPIEKTISKAKGQPREPQVY |
| | | | TLPPCREEMTKNQVSLWCLVKGFYPSDIAVEWESNGQPENNYKTT |
| | | | PPVLDSDGSFFLYSKLTVDKSRWQQGNVFSCSVMHEALHNHYTQ |
| | | | KSLSLSPGK |
| | 2270 | IL-2 C125A | APTSSSTKKTQLQLEHLLLDLQMILNGINNYKNPKLTRMLTFKFY |
| | | | MPKKATELKHLQCLEEELKPLEEVLNLAQSKNFHLRPRDLISNINV |
| | | | IVLELKGSETTFMCEYADETATIVEFLNRWITFAQSIISTLT |
| | 3308 | linker | GGGGSGGGGS |
| | 3648 | Human Fc Knob | DKTHTCPPCPAPELLGGPSVFLFPPKPKDTLMISRTPEVTCVVVDV |
| | | cys N297A | SHEDPEVKFNWYVDGVEVHNAKTKPREEQYASTYRVVSVLTVLH |
| | | | QDWLNGKEYKCKVSNKALPAPIEKTISKAKGQPREPQVYTLPPCR |
| | | | EEMTKNQVSLWCLVKGFYPSDIAVEWESNGQPENNYKTTPPVLD |
| | | | SDGSFFLYSKLTVDKSRWQQGNVFSCSVMHEALHNHYTQKSLSLS |
| | | | PGK |
| Chain 3 | 4009 | Full Sequence | DIQMTQSPSFLSASVGDRVTITCKASQNVGNRVAWYQQKPGKAP |
| (light chain) | | | KALIYSSSHRYKGVPSRFSGSGSGTEFTLTISSLQPEDFATYFCQQF |
| (CDRs | | | KSYPLTFGQGTKLEIKRTVAAPSVFIFPPSDEQLKSGTASVVCLLNN |
| underlined) | | | FYPREAKVQWKVDNALQSGNSQESVTEQDSKDSTYSLSSTLTLSK |
| | | | ADYEKHKVYACEVTHQGLSSPVTKSFNRGEC |
| | 108 | anti-TCRvβ VL | DIQMTQSPSFLSASVGDRVTITCKASQNVGNRVAWYQQKPGKAP |
| | | | KALIYSSSHRYKGVPSRFSGSGSGTEFTLTISSLQPEDFATYFCQQF |
| | | | KSYPLTFGQGTKLEIK |
| | 3644 | Immunoglobulin | ALQSGNSQESVTEQDSKDSTYSLSSTLTLSKADYEKHKVYACEVT |
| | | kappa constant C | RTVAAPSVFIFPPSDEQLKSGTASVVCLLNNFYPREAKVQWKVDNH |
| | | region | QGLSSPVTKSFNRGEC |

Humanized Molecule 6

| | SEQ | | |
|---|---|---|---|
| | ID | | |
| | NO: | | |
| Chain 1 | 4010 | Full Sequence | QVQLVQSGAEVKKPGSSVKVSCKASGTDFDKIYIHWVRQAPGQG |
| (heavy chain) | | | LEWMGRVSAGSGNTKYNEKFKGRVTITADTSTSTAYMELSSLRSE |
| (CDRs | | | DTAVYYCAGSYYSYDVLDYWGQGTTVTVSSASTKGPSVFPLAPSS |
| underlined) | | | KSTSGGTAALGCLVKDYFPEPVTVSWNSGALTSGVHTFPAVLQSS |
| | | | GLYSLSSVVTVPSSSLGTQTYICNVNHKPSNTKVDKRVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYASTYRVVSVLTVLHQD |
| | | | WLNGKEYKCKVSNKALPAPIEKTISKAKGQPREPQVCTLPPSREE |
| | | | MTKNQVSLSCAVKGFYPSDIAVEWESNGQPENNYKTTPPVLDSDG |
| | | | SFFLVSKLTVDKSRWQQGNVFSCSVMHEALHNHYTQKSLSLSP |
| | 109 | anti-TCRvβ VH | QVQLVQSGAEVKKPGSSVKVSCKASGTDFDKIYIHWVRQAPGQG |
| | | | LEWMGRVSAGSGNTKYNEKFKGRVTITADTSTSTAYMELSSLRSE |
| | | | DTAVYYCAGSYYSYDVLDYWGQGTTVTVSS |
| | 4001 | Human IgG1 | ASTKGPSVFPLAPSSKSTSGGTAALGCLVKDYFPEPVTVSWNSGAL |
| | | hole cys N297A | TSGVHTFPAVLQSSGLYSLSSVVTVPSSSLGTQTYICNVNHKPSNT |
| | | | KVDKRVEPKSCDKTHTCPPCPAPELLGGPSVFLFPPKPKDTLMISR |
| | | | TPEVTCVVVDVSHEDPEVKFNWYVDGVEVHNAKTKPREEQYAST |
| | | | YRVVSVLTVLHQDWLNGKEYKCKVSNKALPAPIEKTISKAKGQPR |
| | | | EPQVCTLPPSREEMTKNQVSLSCAVKGFYPSDIAVEWESNGQPEN |
| | | | NYKTTPPVLDSDGSFFLVSKLTVDKSRWQQGNVFSCSVMHEALH |
| | | | NHYTQKSLSLSP |
| Chain 2 | 4003 | Full Sequence | APTSSSTKKTQLQLEHLLLDLQMILNGINNYKNPKLTRMLTFKFY |
| (IL2-Fc) | | | MPKKATELKHLQCLEEELKPLEEVLNLAQSKNFHLRPRDLISNINV |
| | | | IVLELKGSETTFMCEYADETATIVEFLNRWITFAQSIISTLTGGGGS |
| | | | GGGGSDKTHTCPPCPAPELLGGPSVFLFPPKPKDTLMISRTPEVTC |
| | | | VVVDVSHEDPEVKFNWYVDGVEVHNAKTKPREEQYASTYRVVS |
| | | | VLTVLHQDWLNGKEYKCKVSNKALPAPIEKTISKAKGQPREPQVY |
| | | | TLPPCREEMTKNQVSLWCLVKGFYPSDIAVEWESNGQPENNYKTT |
| | | | PPVLDSDGSFFLYSKLTVDKSRWQQGNVFSCSVMHEALHNHYTQ |
| | | | KSLSLSP |
| | 2270 | IL-2 C125A | APTSSSTKKTQLQLEHLLLDLQMILNGINNYKNPKLTRMLTFKFY |
| | | | MPKKATELKHLQCLEEELKPLEEVLNLAQSKNFHLRPRDLISNINV |
| | | | IVLELKGSETTFMCEYADETATIVEFLNRWITFAQSIISTLT |
| | 3308 | linker | GGGGSGGGGS |
| | 3648 | Human Fc Knob | DKTHTCPPCPAPELLGGPSVFLFPPKPKDTLMISRTPEVTCVVVDV |
| | | cys N297A | SHEDPEVKFNWYVDGVEVHNAKTKPREEQYASTYRVVSVLTVLH |
| | | | QDWLNGKEYKCKVSNKALPAPIEKTISKAKGQPREPQVYTLPPCR |
| | | | EEMTKNQVSLWCLVKGFYPSDIAVEWESNGQPENNYKTTPPVLD |
| | | | SDGSFFLYSKLTVDKSRWQQGNVFSCSVMHEALHNHYTQKSLSLS |
| | | | PGK |
| Chain 3 | 4009 | Full Sequence | DIQMTQSPSFLSASVGDRVTITCKASQNVGNRVAWYQQKPGKAP |
| (light chain) | | | KALIYSSSHRYKGVPSRFSGSGSGTEFTLTISSLQPEDFATYFCQQF |
| (CDRs | | | KSYPLTFGQGTKLEIKRTVAAPSVFIFPPSDEQLKSGTASVVCLLNN |
| underlined) | | | FYPREAKVQWKVDNALQSGNSQESVTEQDSKDSTYSLSSTLTLSK |
| | | | ADYEKHKVYACEVTHQGLSSPVTKSFNRGEC |
| | 108 | anti-TCRvβ VL | DIQMTQSPSFLSASVGDRVTITCKASQNVGNRVAWYQQKPGKAP |
| | | | KALIYSSSHRYKGVPSRFSGSGSGTEFTLTISSLQPEDFATYFCQQF |
| | | | KSYPLTFGQGTKLEIK |
| | 3644 | Immunoglobulin | ALQSGNSQESVTEQDSKDSTYSLSSTLTLSKADYEKHKVYACEVT |
| | | kappa constant | RTVAAPSVFIFPPSDEQLKSGTASVVCLLNNFYPREAKVQWKVDNH |
| | | C region | QGLSSPVTKSFNRGEC |

Humanized Molecule 7

| | SEQ | | |
|---|---|---|---|
| | ID | | |
| | NO: | | |
| Chain 1 | 4011 | Full Sequence | QVQLVQSGAEVKKPGSSVKVSCKASGHDFKLTYIHWVRQAPGQG |
| (heavy chain) | | | LEWMGRVSPGSGNTKYNEKFKGRVTITADTSTSTAYMELSSLRSE |
| (CDRs | | | DTAVYYCAGSYYSYDVLDYWGQGTTVTVSSASTKGPSVFPLAPSS |
| underlined) | | | KSTSGGTAALGCLVKDYFPEPVTVSWNSGALTSGVHTFPAVLQSS |
| | | | GLYSLSSVVTVPSSSLGTQTYICNVNHKPSNTKVDKRVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYASTYRVVSVLTVLHQD |
| | | | WLNGKEYKCKVSNKALPAPIEKTISKAKGQPREPQVCTLPPSREE |
| | | | MTKNQVSLSCAVKGFYPSDIAVEWESNGQPENNYKTTPPVLDSDG |
| | | | SFFLVSKLTVDKSRWQQGNVFSCSVMHEALHNHYTQKSLSLSPGK |
| | 182 | anti-TCRvβ VH | QVQLVQSGAEVKKPGSSVKVSCKASGHDFKLTYIHWVRQAPGQG |
| | | | LEWMGRVSPGSGNTKYNEKFKGRVTITADTSTSTAYMELSSLRSE |
| | | | DTAVYYCAGSYYSYDVLDYWGQGTTVTVSS |
| | 3649 | Human IgG1 | ASTKGPSVFPLAPSSKSTSGGTAALGCLVKDYFPEPVTVSWNSGAL |
| | | hole cys N297A | TSGVHTFPAVLQSSGLYSLSSVVTVPSSSLGTQTYICNVNHKPSNT |
| | | | KVDKRVEPKSCDKTHTCPPCPAPELLGGPSVFLFPPKPKDTLMISR |
| | | | TPEVTCVVVDVSHEDPEVKFNWYVDGVEVHNAKTKPREEQYAST |
| | | | YRVVSVLTVLHQDWLNGKEYKCKVSNKALPAPIEKTISKAKGQPR |
| | | | EPQVCTLPPSREEMTKNQVSLSCAVKGFYPSDIAVEWESNGQPEN |
| | | | NYKTTPPVLDSDGSFFLVSKLTVDKSRWQQGNVFSCSVMHEALH |
| | | | NHYTQKSLSLSPGK |
| Chain 2 | 4013 | Full Sequence | APTSSSTKKTQLQLEHLLLDLQMILNGINNYKNPKLTRMLTFKFY |
| (IL2-Fc) | | | MPKKATELKHLQCLEEELKPLEEVLNLAQSKNFHLRPRDLISNINV |
| | | | IVLELKGSETTFMCEYADETATIVEFLNRWITFCQSIISTLTGGGGS |
| | | | GGGGSDKTHTCPPCPAPELLGGPSVFLFPPKPKDTLMISRTPEVTC |
| | | | VVVDVSHEDPEVKFNWYVDGVEVHNAKTKPREEQYASTYRVVS |
| | | | VLTVLHQDWLNGKEYKCKVSNKALPAPIEKTISKAKGQPREPQVY |
| | | | TLPPCREEMTKNQVSLWCLVKGFYPSDIAVEWESNGQPENNYKTT |
| | | | PPVLDSDGSFFLYSKLTVDKSRWQQGNVFSCSVMHEALHNHYTQ |
| | | | KSLSLSPGK |
| | 2191 | IL-2 | APTSSSTKKTQLQLEHLLLDLQMILNGINNYKNPKLTRMLTFKFY |
| | | | MPKKATELKHLQCLEEELKPLEEVLNLAQSKNFHLRPRDLISNINV |
| | | | IVLELKGSETTFMCEYADETATIVEFLNRWITFCQSIISTLT |
| | 3308 | linker | GGGGGGGGS |
| | 3648 | Human Fc Knob | DKTHTCPPCPAPELLGGPSVFLFPPKPKDTLMISRTPEVTCVVVDV |
| | | cys N297A | SHEDPEVKFNWYVDGVEVHNAKTKPREEQYASTYRVVSVLTVLH |
| | | | QDWLNGKEYKCKVSNKALPAPIEKTISKAKGQPREPQVYTLPPCR |
| | | | EEMTKNQVSLWCLVKGFYPSDIAVEWESNGQPENNYKTTPPVLD |
| | | | SDGSFFLYSKLTVDKSRWQQGNVFSCSVMHEALHNHYTQKSLSLS |
| | | | PGK |
| Chain 3 | 4012 | Full Sequence | DIQMTQSPSFLSASVGDRVTITCKASQNVEDRVAWHQQKPGKAPK |
| light chain) | | | ALIYSSSHRYKGVPSRFSGSGSGTEFTLTISSLQPEDFATYFCQQFKS |
| (CDRs | | | YPLTFGQGTKLEIKRTVAAPSVFIFPPSDEQLKSGTASVVCLLNNFY |
| underlined) | | | PREAKVQWKVDNALQSGNSQESVTEQDSKDSTYSLSSTLTLSKAD |
| | | | YEKHKVYACEVTHQGLSSPVTKSFNRGEC |
| | 181 | anti-TCRvβ VL | DIQMTQSPSFLSASVGDRVTITCKASQNVEDRVAWHQQKPGKAPK |
| | | | ALIYSSSHRYKGVPSRFSGSGSGTEFTLTISSLQPEDFATYFCQQFKS |
| | | | YPLTFGQGTKLEIK |
| | 3644 | Immunoglobulin | ALQSGNSQESVTEQDSKDSTYSLSSTLTLSKADYEKHKVYACEVT |
| | | kappa constant | RTVAAPSVFIFPPSDEQLKSGTASVVCLLNNFYPREAKVQWKVDNH |
| | | C region | QGLSSPVTKSFNRGEC |

Humanized Molecule 8

| | SEQ | | |
|---|---|---|---|
| | ID | | |
| | NO: | | |
| Chain 1 | 4014 | Full Sequence | QVQLVQSGAEVKKPGSSVKVSCKASGHDFKLTYIHWVRQAPGQG |
| (heavy chain) | | | LEWMGRVSPGSGNTKYNEKFKGRVTITADTSTSTAYMELSSLRSE |
| (CDRs | | | DTAVYYCAGSYYSYDVLDYWGQGTTVTVSSASTKGPSVFPLAPSS |
| underlined) | | | KSTSGGTAALGCLVKDYFPEPVTVSWNSGALTSGVHTFPAVLQSS |
| | | | GLYSLSSVVTVPSSSLGTQTYICNVNHKPSNTKVDKRVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYASTYRVVSVLTVLHQD |
| | | | WLNGKEYKCKVSNKALPAPIEKTISKAKGQPREPQVCTLPPSREE |
| | | | MTKNQVSLSCAVKGFYPSDIAVEWESNGQPENNYKTTPPVLDSDG |
| | | | SFFLVSKLTVDKSRWQQGNVFSCSVMHEALHNHYTQKSLSLSP |
| | 182 | anti-TCRvβ VH | QVQLVQSGAEVKKPGSSVKVSCKASGHDFKLTYIHWVRQAPGQG |
| | | | LEWMGRVSPGSGNTKYNEKFKGRVTITADTSTSTAYMELSSLRSE |
| | | | DTAVYYCAGSYYSYDVLDYWGQGTTVTVSS |
| | 4001 | Human IgG1 | ASTKGPSVFPLAPSSKSTSGGTAALGCLVKDYFPEPVTVSWNSGAL |
| | | hole cys N297A | TSGVHTFPAVLQSSGLYSLSSVVTVPSSSLGTQTYICNVNHKPSNT |
| | | | KVDKRVEPKSCDKTHTCPPCPAPELLGGPSVFLFPPKPKDTLMISR |
| | | | TPEVTCVVVDVSHEDPEVKFNWYVDGVEVHNAKTKPREEQYAST |
| | | | YRVVSVLTVLHQDWLNGKEYKCKVSNKALPAPIEKTISKAKGQPR |
| | | | EPQVCTLPPSREEMTKNQVSLSCAVKGFYPSDIAVEWESNGQPEN |
| | | | NYKTTPPVLDSDGSFFLVSKLTVDKSRWQQGNVFSCSVMHEALH |
| | | | NHYTQKSLSLSP |
| Chain 2 | 4015 | Full Sequence | APTSSSTKKTQLQLEHLLLDLQMILNGINNYKNPKLTRMLTFKFY |
| (IL2-Fc) | | | MPKKATELKHLQCLEEELKPLEEVLNLAQSKNFHLRPRDLISNINV |
| | | | IVLELKGSETTFMCEYADETATIVEFLNRWITFCQSIISTLTGGGGS |
| | | | GGGGSDKTHTCPPCPAPELLGGPSVFLFPPKPKDTLMISRTPEVTC |
| | | | VVVDVSHEDPEVKFNWYVDGVEVHNAKTKPREEQYASTYRVVS |
| | | | VLTVLHQDWLNGKEYKCKVSNKALPAPIEKTISKAKGQPREPQVY |
| | | | TLPPCREEMTKNQVSLWCLVKGFYPSDIAVEWESNGQPENNYKTT |
| | | | PPVLDSDGSFFLYSKLTVDKSRWQQGNVFSCSVMHEALHNHYTQ |
| | | | KSLSLSP |
| | 2191 | IL-2 | APTSSSTKKTQLQLEHLLLDLQMILNGINNYKNPKLTRMLTFKFY |
| | | | MPKKATELKHLQCLEEELKPLEEVLNLAQSKNFHLRPRDLISNINV |
| | | | IVLELKGSETTFMCEYADETATIVEFLNRWITFCQSIISTLT |
| | 3308 | linker | GGGGSGGGGS |
| | 4031 | Human Fc Knob | DKTHTCPPCPAPELLGGPSVFLFPPKPKDTLMISRTPEVTCVVVDV |
| | | cys N297A | SHEDPEVKFNWYVDGVEVHNAKTKPREEQYASTYRVVSVLTVLH |
| | | | QDWLNGKEYKCKVSNKALPAPIEKTISKAKGQPREPQVYTLPPCR |
| | | | EEMTKNQVSLWCLVKGFYPSDIAVEWESNGQPENNYKTTPPVLD |
| | | | SDGSFFLYSKLTVDKSRWQQGNVFSCSVMHEALHNHYTQKSLSLS |
| | | | P |
| Chain 3 | 4012 | Full Sequence | DIQMTQSPSFLSASVGDRVTITCKASQNVEDRVAWHQQKPGKAPK |
| (light chain) | | | ALIYSSSHRYKGVPSRFSGSGSGTEFTLTISSLQPEDFATYFCQQFKS |
| (CDRs | | | YPLTFGQGTKLEIKRTVAAPSVFIFPPSDEQLKSGTASVVCLLNNFY |
| underlined) | | | PREAKVQWKVDNALQSGNSQESVTEQDSKDSTYSLSSTLTLSKAD |
| | | | YEKHKVYACEVTHQGLSSPVTKSFNRGEC |
| | 181 | anti-TCRvβ VL | DIQMTQSPSFLSASVGDRVTITCKASQNVEDRVAWHQQKPGKAPK |
| | | | ALIYSSSHRYKGVPSRFSGSGSGTEFTLTISSLQPEDFATYFCQQFKS |
| | | | YPLTFGQGTKLEIK |
| | 3644 | Immunoglobulin | ALQSGNSQESVTEQDSKDSTYSLSSTLTLSKADYEKHKVYACEVT |
| | | kappa constant | RTVAAPSVFIFPPSDEQLKSGTASVVCLLNNFYPREAKVQWKVDNH |
| | | C region | QGLSSPVTKSFNRGEC |

Humanized Molecule 9

| | SEQ | | |
|---|---|---|---|
| | ID | | |
| | NO: | | |
| Chain 1 | 4016 | Full Sequence | QVQLVQSGAEVKKPGSSVKVSCKASGTDFDKTYIHWVRQAPGQG |
| (heavy chain) | | | LEWMGRISAGSGNVKYNEKFKGRVTITADTSTSTAYMELSSLRSE |
| (CDRs | | | DTAVYYCAGSYYSYDVLDYWGQGTTVTVSSASTKGPSVFPLAPSS |
| underlined) | | | KSTSGGTAALGCLVKDYFPEPVTVSWNSGALTSGVHTFPAVLQSS |
| | | | GLYSLSSVVTVPSSSLGTQTYICNVNHKPSNTKVDKRVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYASTYRVVSVLTVLHQD |
| | | | WLNGKEYKCKVSNKALPAPIEKTISKAKGQPREPQVCTLPPSREE |
| | | | MTKNQVSLSCAVKGFYPSDIAVEWESNGQPENNYKTTPPVLDSDG |
| | | | SFFLVSKLTVDKSRWQQGNVFSCSVMHEALHNHYTQKSLSLSPGK |
| | 4029 | anti-TCRvβ VH | QVQLVQSGAEVKKPGSSVKVSCKASGTDFDKTYIHWVRQAPGQG |
| | | | LEWMGRISAGSGNVKYNEKFKGRVTITADTSTSTAYMELSSLRSE |
| | | | DTAVYYCAGSYYSYDVLDYWGQGTTVTVSS |
| | 3649 | Human IgG1 | ASTKGPSVFPLAPSSKSTSGGTAALGCLVKDYFPEPVTVSWNSGAL |
| | | hole cys N297A | TSGVHTFPAVLQSSGLYSLSSVVTVPSSSLGTQTYICNVNHKPSNT |
| | | | KVDKRVEPKSCDKTHTCPPCPAPELLGGPSVFLFPPKPKDTLMISR |
| | | | TPEVTCVVVDVSHEDPEVKFNWYVDGVEVHNAKTKPREEQYAST |
| | | | YRVVSVLTVLHQDWLNGKEYKCKVSNKALPAPIEKTISKAKGQPR |
| | | | EPQVCTLPPSREEMTKNQVSLSCAVKGFYPSDIAVEWESNGQPEN |
| | | | NYKTTPPVLDSDGSFFLVSKLTVDKSRWQQGNVFSCSVMHEALH |
| | | | NHYTQKSLSLSPGK |
| Chain 2 | 3521 | Full Sequence | APTSSSTKKTQLQLEHLLLDLQMILNGINNYKNPKLTRMLTFKFY |
| (IL2-Fc) | | | MPKKATELKHLQCLEEELKPLEEVLNLAQSKNFHLRPRDLISNINV |
| | | | IVLELKGSETTFMCEYADETATIVEFLNRWITFAQSIISTLTGGGGS |
| | | | GGGGSDKTHTCPPCPAPELLGGPSVFLFPPKPKDTLMISRTPEVTC |
| | | | VVVDVSHEDPEVKFNWYVDGVEVHNAKTKPREEQYASTYRVVS |
| | | | VLTVLHQDWLNGKEYKCKVSNKALPAPIEKTISKAKGQPREPQVY |
| | | | TLPPCREEMTKNQVSLWCLVKGFYPSDIAVEWESNGQPENNYKTT |
| | | | PPVLDSDGSFFLYSKLTVDKSRWQQGNVFSCSVMHEALHNHYTQ |
| | | | KSLSLSPGK |
| | 2270 | IL-2 C125A | APTSSSTKKTQLQLEHLLLDLQMILNGINNYKNPKLTRMLTFKFY |
| | | | MPKKATELKHLQCLEEELKPLEEVLNLAQSKNFHLRPRDLISNINV |
| | | | IVLELKGSETTFMCEYADETATIVEFLNRWITFAQSIISTLT |
| | 3308 | linker | GGGGGGGGS |
| | 3648 | Human Fc Knob | DKTHTCPPCPAPELLGGPSVFLFPPKPKDTLMISRTPEVTCVVVDV |
| | | cys N297A | SHEDPEVKFNWYVDGVEVHNAKTKPREEQYASTYRVVSVLTVLH |
| | | | QDWLNGKEYKCKVSNKALPAPIEKTISKAKGQPREPQVYTLPPCR |
| | | | EEMTKNQVSLWCLVKGFYPSDIAVEWESNGQPENNYKTTPPVLD |
| | | | SDGSFFLYSKLTVDKSRWQQGNVFSCSVMHEALHNHYTQKSLSLS |
| | | | PGK |
| Chain 3 | 4017 | Full Sequence | DIQMTQSPSFLSASVGDRVTITCKASQNVDDRVAWYQQKPGKAP |
| (light chain) | | | KALIYSSSHRYKGVPSRFSGSGSGTEFTLTISSLQPEDFATYFCQQF |
| (CDRs | | | KSYPLTFGQGTKLEIKRTVAAPSVFIFPPSDEQLKSGTASVVCLLNN |
| underlined) | | | FYPREAKVQWKVDNALQSGNSQESVTEQDSKDSTYSLSSTLTLSK |
| | | | ADYEKHKVYACEVTHQGLSSPVTKSFNRGEC |
| | 187 | anti-TCRvβ VL | DIQMTQSPSFLSASVGDRVTITCKASQNVDDRVAWYQQKPGKAP |
| | | | KALIYSSSHRYKGVPSRFSGSGSGTEFTLTISSLQPEDFATYFCQQF |
| | | | KSYPLTFGQGTKLEIK |
| | 3644 | Immunoglobulin | RTVAAPSVFIFPPSDEQLKSGTASVVCLLNNFYPREAKVQWKVDN |
| | | kappa constant C | ALQSGNSQESVTEQDSKDSTYSLSSTLTLSKADYEKHKVYACEVT |
| | | region | HQGLSSPVTKSFNRGEC |

Humanized Molecule 10

| | SEQ | | |
|---|---|---|---|
| | ID | | |
| | NO: | | |
| Chain 1 | 4018 | Full Sequence | QVQLVQSGAEVKKPGSSVKVSCKASGTDFDKTYIHWVRQAPGQG |
| (heavy chain) | | | LEWMGRISAGSGNVKYNEKFKGRVTITADTSTSTAYMELSSLRSE |
| (CDRs | | | DTAVYYCAGSYYSYDVLDYWGQGTTVTVSSASTKGPSVFPLAPSS |
| underlined) | | | KSTSGGTAALGCLVKDYFPEPVTVSWNSGALTSGVHTFPAVLQSS |
| | | | GLYSLSSVVTVPSSSLGTQTYICNVNHKPSNTKVDKRVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYASTYRVVSVLTVLHQD |
| | | | WLNGKEYKCKVSNKALPAPIEKTISKAKGQPREPQVCTLPPSREE |
| | | | MTKNQVSLSCAVKGFYPSDIAVEWESNGQPENNYKTTPPVLDSDG |
| | | | SFFLVSKLTVDKSRWQQGNVFSCSVMHEALHNHYTQKSLSLSP |
| | 4029 | anti-TCRvβ VH | QVQLVQSGAEVKKPGSSVKVSCKASGTDFDKTYIHWVRQAPGQG |
| | | | LEWMGRISAGSGNVKYNEKFKGRVTITADTSTSTAYMELSSLRSE |
| | | | DTAVYYCAGSYYSYDVLDYWGQGTTVTVSS |
| | 4001 | Human IgG1 | ASTKGPSVFPLAPSSKSTSGGTAALGCLVKDYFPEPVTVSWNSGAL |
| | | hole cys N297A | TSGVHTFPAVLQSSGLYSLSSVVTVPSSSLGTQTYICNVNHKPSNT |
| | | | KVDKRVEPKSCDKTHTCPPCPAPELLGGPSVFLFPPKPKDTLMISR |
| | | | TPEVTCVVVDVSHEDPEVKFNWYVDGVEVHNAKTKPREEQYAST |
| | | | YRVVSVLTVLHQDWLNGKEYKCKVSNKALPAPIEKTISKAKGQPR |
| | | | EPQVCTLPPSREEMTKNQVSLSCAVKGFYPSDIAVEWESNGQPEN |
| | | | NYKTTPPVLDSDGSFFLVSKLTVDKSRWQQGNVFSCSVMHEALH |
| | | | NHYTQKSLSLSP |
| Chain 2 | 4002 | Full Sequence | APTSSSTKKTQLQLEHLLLDLQMILNGINNYKNPKLTRMLTFKFY |
| (IL2-Fc) | | | MPKKATELKHLQCLEEELKPLEEVLNLAQSKNFHLRPRDLISNINV |
| | | | IVLELKGSETTFMCEYADETATIVEFLNRWITFAQSIISTLTGGGGS |
| | | | GGGGSDKTHTCPPCPAPELLGGPSVFLFPPKPKDTLMISRTPEVTC |
| | | | VVVDVSHEDPEVKFNWYVDGVEVHNAKTKPREEQYASTYRVVS |
| | | | VLTVLHQDWLNGKEYKCKVSNKALPAPIEKTISKAKGQPREPQVY |
| | | | TLPPCREEMTKNQVSLWCLVKGFYPSDIAVEWESNGQPENNYKTT |
| | | | PPVLDSDGSFFLYSKLTVDKSRWQQGNVFSCSVMHEALHNHYTQ |
| | | | KSLSLSP |
| | 2270 | IL-2 C125A | APTSSSTKKTQLQLEHLLLDLQMILNGINNYKNPKLTRMLTFKFY |
| | | | MPKKATELKHLQCLEEELKPLEEVLNLAQSKNFHLRPRDLISNINV |
| | | | IVLELKGSETTFMCEYADETATIVEFLNRWITFAQSIISTLT |
| | 3308 | linker | GGGGSGGGGS |
| | 4031 | Human Fc Knob | DKTHTCPPCPAPELLGGPSVFLFPPKPKDTLMISRTPEVTCVVVDV |
| | | cys N297A | SHEDPEVKFNWYVDGVEVHNAKTKPREEQYASTYRVVSVLTVLH |
| | | | QDWLNGKEYKCKVSNKALPAPIEKTISKAKGQPREPQVYTLPPCR |
| | | | EEMTKNQVSLWCLVKGFYPSDIAVEWESNGQPENNYKTTPPVLD |
| | | | SDGSFFLYSKLTVDKSRWQQGNVFSCSVMHEALHNHYTQKSLSLS |
| | | | p |
| Chain 3 | 4017 | Full Sequence | DIQMTQSPSFLSASVGDRVTITCKASQNVDDRVAWYQQKPGKAP |
| (light chain) | | | KALIYSSSHRYKGVPSRFSGSGSGTEFTLTISSLQPEDFATYFCQQF |
| (CDRs | | | KSYPLTFGQGTKLEIKRTVAAPSVFIFPPSDEQLKSGTASVVCLLNN |
| underlined) | | | FYPREAKVQWKVDNALQSGNSQESVTEQDSKDSTYSLSSTLTLSK |
| | | | ADYEKHKVYACEVTHQGLSSPVTKSENRGEC |
| | 187 | anti-TCRvβ VL | DIQMTQSPSFLSASVGDRVTITCKASQNVDDRVAWYQQKPGKAP |
| | | | KALIYSSSHRYKGVPSRFSGSGSGTEFTLTISSLQPEDFATYFCQQF |
| | | | KSYPLTFGQGTKLEIK |
| | 3644 | Immunoglobulin | RTVAAPSVFIFPPSDEQLKSGTASVVCLLNNFYPREAKVQWKVDN |
| | | kappa constant C | ALQSGNSQESVTEQDSKDSTYSLSSTLTLSKADYEKHKVYACEVT |
| | | region | HQGLSSPVTKSFNRGEC |

Humanized Molecule 11

| | SEQ | |
|---|---|---|
| | ID | |
| | NO: | |
| Chain 1 | 4019 | QVQLVQSGAEVKKPGSSVKVSCKASGHDFRLTYIHWVRQAPGQGLEWMGRVSAG |
| | | SGNVKYNEKFKGRVTITADTSTSTAYMELSSLRSEDTAVYYCAVSYYSYDVLDYW |
| | | GQGTTVTVSSGGGGSGGGGSGGGGSGGGGSDIQMTQSPSFLSASVGDRVTITCKAS |
| | | QNVADRVVWHQQKPGKAPKALIYSSSHRYKGVPSRFSGSGSGTEFTLTISSLQPEDF |
| | | ATYFCQQFKSYPLTFGQGTKLEIKASTKGPSVFPLAPSSKSTSGGTAALGCLVKDYF |
| | | PEPVTVSWNSGALTSGVHTFPAVLQSSGLYSLSSVVTVPSSSLGTQTYICNVNHKPS |
| | | NTKVDKRVEPKSCDKTHTCPPCPAPELLGGPSVFLFPPKPKDTLMISRTPEVTCVVV |
| | | DVSHEDPEVKFNWYVDGVEVHNAKTKPREEQYASTYRVVSVLTVLHQDWLNGKE |
| | | YKCKVSNKALPAPIEKTISKAKGQPREPQVCTLPPSREEMTKNQVSLSCAVKGFYPS |
| | | DIAVEWESNGQPENNYKTTPPVLDSDGSFFLVSKLTVDKSRWQQGNVFSCSVMHE |
| | | ALHNHYTQKSLSLSP |
| Chain 2 | 4020 | APTSSSTKKTQLQLEHLLLDLQMILNGINNYKNPKLTRMLTFKFYMPKKATELKHL |
| | | QCLEEELKPLEEVLNLAQSKNFHLRPRDLISNINVIVLELKGSETTFMCEYADETATI |
| | | VEFLNRWITFAQSIISTLTGGGGSGGGGSDKTHTCPPCPAPELLGGPSVFLFPPKPKD |
| | | TLMISRTPEVTCVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPCREEMTK |
| | | NQVSLWCLVKGFYPSDIAVEWESNGQPENNYKTTPPVLDSDGSFFLYSKLTVDKSR |
| | | WQQGNVFSCSVMHEALHNHYTQKSLSLSP |

Humanized Molecule 12

| | SEQ | |
|---|---|---|
| | ID | |
| | NO: | |
| Chain 1 | 4021 | QVQLVQSGAEVKKPGSSVKVSCKASGYSFTTYYIHWVRQAPGQGLEWMGWFFPG |
| | | SGNIKYNEKFKGRVTITADTSTSTAYMELSSLRSEDTAVYYCAGSYYSYDVLDYWG |
| | | QGTTVTVSSGGGGGGGGSGGGGSGGGGSDIQMTQSPSSLSASVGDRVTITCKASQ |
| | | NVGINVVWHQQKPGKVPKALIYSSSHRYSGVPSRFSGSGSGTDFTLTISSLQPEDVA |
| | | TYFCQQFKSYPLTFGQGTKLEIKASTKGPSVFPLAPSSKSTSGGTAALGCLVKDYFPE |
| | | PVTVSWNSGALTSGVHTFPAVLQSSGLYSLSSVVTVPSSSLGTQTYICNVNHKPSNT |
| | | KVDKRVEPKSCDKTHTCPPCPAPELLGGPSVFLFPPKPKDTLMISRTPEVTCVVVDV |
| | | SHEDPEVKFNWYVDGVEVHNAKTKPREEQYASTYRVVSVLTVLHQDWLNGKEYK |
| | | CKVSNKALPAPIEKTISKAKGQPREPQVCTLPPSREEMTKNQVSLSCAVKGFYPSDI |
| | | AVEWESNGQPENNYKTTPPVLDSDGSFFLVSKLTVDKSRWQQGNVFSCSVMHEAL |
| | | HNHYTQKSLSLSP |
| Chain 2 | 4022 | APASSSTKKTQLQLEHLLLDLQMILNGINNYKNPKLTRMLTAKFAMPKKATELKHL |
| | | QCLEEELKPLEEVLNGAQSKNFHLRPRDLISNINVIVLELKGSETTFMCEYADETATI |
| | | VEFLNRWITFAQSIISTLTGGGGSGGGGSDKTHTCPPCPAPELLGGPSVFLFPPKPKD |
| | | TLMISRTPEVTCVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPCREEMTK |
| | | NQVSLWCLVKGFYPSDIAVEWESNGQPENNYKTTPPVLDSDGSFFLYSKLTVDKSR |
| | | WQQGNVFSCSVMHEALHNHYTQKSLSLSP |

Humanized Molecule 13

| | SEQ | |
|---|---|---|
| | ID | |
| | NO: | |
| Chain 1 | 4023 | QVQLVQSGAEVKKPGSSVKVSCKASGTDFDKIYIHWVRQAPGQGLEWMGRVSAG |
| | | SGNTKYNEKFKGRVTITADTSTSTAYMELSSLRSEDTAVYYCAGSYYSYDVLDYW |
| | | GQGTTVTVSSGGGGSGGGGSGGGGSGGGGSDIQMTQSPSFLSASVGDRVTITCKAS |
| | | QNVGNRVAWYQQKPGKAPKALIYSSSHRYKGVPSRFSGSGSGTEFTLTISSLQPEDF |
| | | ATYFCQQFKSYPLTFGQGTKLEIKASTKGPSVFPLAPSSKSTSGGTAALGCLVKDYF |
| | | PEPVTVSWNSGALTSGVHTFPAVLQSSGLYSLSSVVTVPSSSLGTQTYICNVNHKPS |
| | | NTKVDKRVEPKSCDKTHTCPPCPAPELLGGPSVFLFPPKPKDTLMISRTPEVTCVVV |
| | | DVSHEDPEVKFNWYVDGVEVHNAKTKPREEQYASTYRVVSVLTVLHQDWLNGKE |
| | | YKCKVSNKALPAPIEKTISKAKGQPREPQVCTLPPSREEMTKNQVSLSCAVKGFYPS |
| | | DIAVEWESNGQPENNYKTTPPVLDSDGSFFLVSKLTVDKSRWQQGNVFSCSVMHE |
| | | ALHNHYTQKSLSLSP |
| Chain 2 | 4024 | APTSSSTKKTQLQLEHLLLDLQMILNGINNYKNPKLTRMLTFKFYMPKKATELKHL |
| | | QCLEEELKPLEEVLNLAQSKNFHLRPRDLISNINVIVLELKGSETTFMCEYADETATI |
| | | VEFLNRWITFAQSIISTLTGGGGSGGGGSDKTHTCPPCPAPELLGGPSVFLFPPKPKD |
| | | TLMISRTPEVTCVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPCREEMTK |
| | | NQVSLWCLVKGFYPSDIAVEWESNGQPENNYKTTPPVLDSDGSFFLYSKLTVDKSR |
| | | WQQGNVFSCSVMHEALHNHYTQKSLSLSP |

Humanized Molecule 14

| | SEQ | |
|---|---|---|
| | ID | |
| | NO: | |
| Chain 1 | 4025 | QVQLVQSGAEVKKPGSSVKVSCKASGHDFKLTYIHWVRQAPGQGLEWMGRVSPG |
| | | SGNTKYNEKFKGRVTITADTSTSTAYMELSSLRSEDTAVYYCAGSYYSYDVLDYW |
| | | GQGTTVTVSSGGGGSGGGGSGGGGSGGGGSDIQMTQSPSFLSASVGDRVTITCKAS |
| | | QNVEDRVAWHQQKPGKAPKALIYSSSHRYKGVPSRFSGSGSGTEFTLTISSLQPEDF |
| | | ATYFCQQFKSYPLTFGQGTKLEIKASTKGPSVFPLAPSSKSTSGGTAALGCLVKDYF |
| | | PEPVTVSWNSGALTSGVHTFPAVLQSSGLYSLSSVVTVPSSSLGTQTYICNVNHKPS |
| | | NTKVDKRVEPKSCDKTHTCPPCPAPELLGGPSVFLFPPKPKDTLMISRTPEVTCVVV |
| | | DVSHEDPEVKFNWYVDGVEVHNAKTKPREEQYASTYRVVSVLTVLHQDWLNGKE |
| | | YKCKVSNKALPAPIEKTISKAKGQPREPQVCTLPPSREEMTKNQVSLSCAVKGFYPS |
| | | DIAVEWESNGQPENNYKTTPPVLDSDGSFFLVSKLTVDKSRWQQGNVFSCSVMHE |
| | | ALHNHYTQKSLSLSP |
| Chain 2 | 4026 | APTSSSTKKTQLQLEHLLLDLQMILNGINNYKNPKLTRMLTFKFYMPKKATELKHL |
| | | QCLEEELKPLEEVLNLAQSKNFHLRPRDLISNINVIVLELKGSETTFMCEYADETATI |
| | | VEFLNRWITFCQSIISTLTGGGGSGGGGSDKTHTCPPCPAPELLGGPSVFLFPPKPKD |
| | | TLMISRTPEVTCVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPCREEMTK |
| | | NQVSLWCLVKGFYPSDIAVEWESNGQPENNYKTTPPVLDSDGSFFLYSKLTVDKSR |
| | | WQQGNVFSCSVMHEALHNHYTQKSLSLSP |

Humanized Molecule 15

| | SEQ | |
|---|---|---|
| | ID | |
| | NO: | |
| Chain 1 | 4027 | QVQLVQSGAEVKKPGSSVKVSCKASGTDFDKTYIHWVRQAPGQGLEWMGRISAGS |
| | | GNVKYNEKFKGRVTITADTSTSTAYMELSSLRSEDTAVYYCAGSYYSYDVLDYWG |
| | | QGTTVTVSSGGGGSGGGGSGGGGSGGGGSDIQMTQSPSFLSASVGDRVTITCKASQ |
| | | NVDDRVAWYQQKPGKAPKALIYSSSHRYKGVPSRFSGSGSGTEFTLTISSLQPEDFA |
| | | TYFCQQFKSYPLTFGQGTKLEIKASTKGPSVFPLAPSSKSTSGGTAALGCLVKDYFPE |
| | | PVTVSWNSGALTSGVHTFPAVLQSSGLYSLSSVVTVPSSSLGTQTYICNVNHKPSNT |
| | | KVDKRVEPKSCDKTHTCPPCPAPELLGGPSVFLFPPKPKDTLMISRTPEVTCVVVDV |
| | | SHEDPEVKFNWYVDGVEVHNAKTKPREEQYASTYRVVSVLTVLHQDWLNGKEYK |
| | | CKVSNKALPAPIEKTISKAKGQPREPQVCTLPPSREEMTKNQVSLSCAVKGFYPSDI |
| | | AVEWESNGQPENNYKTTPPVLDSDGSFFLVSKLTVDKSRWQQGNVFSCSVMHEAL |
| | | HNHYTQKSLSLSP |
| Chain 2 | 4028 | APTSSSTKKTQLQLEHLLLDLQMILNGINNYKNPKLTRMLTFKFYMPKKATELKHL |
| | | QCLEEELKPLEEVLNLAQSKNFHLRPRDLISNINVIVLELKGSETTFMCEYADETATI |
| | | VEFLNRWITFAQSHISTLTGGGGSGGGGSDKTHTCPPCPAPELLGGPSVFLFPPKPKD |
| | | TLMISRTPEVTCVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPCREEMTK |
| | | NQVSLWCLVKGFYPSDIAVEWESNGQPENNYKTTPPVLDSDGSFFLYSKLTVDKSR |
| | | WQQGNVFSCSVMHEALHNHYTQKSLSLSP |

### EXAMPLES

The present disclosure will be more specifically illustrated by the following Examples. However, it should be understood that the present disclosure is not limited by these examples in any manner.

### Example 1: Preclinical Studies

### Investigational Product:

The investigational product for the preclinical studies described in this example is Compound 1, a bifunctional antibody-fusion molecule comprising an antibody Fab region that binds to a subset of human ap T cells expressing the germline-encoded variable beta 6 and 10 (Vβ6/Vβ10) chains of the T cell receptor (TCR) and a physiologically active, unmodified human IL-2 molecule (the other binding arm) fused to the IgG1 antibody Fc domain containing an N297A mutation to prevent interaction with Fc receptors.

### Compound 1 Non-Clinical Pharmacology

### (A) In Vitro Pharmacology

Compound 1 showed high-affinity binding to both human Vβ6-5 and cynomolgus Vβ6-2 TCRs (the dominant TCRs targeted by Compound 1 and representative of the predominant Vβ6 TCR variants expanded by Compound 1 in respective species). In addition. Compound 1 shows comparable binding to both human and Cynomolgus IL-2Rα and IL-2Rβγ heterodimers. In vitro stimulation of human T cells with Compound 1 demonstrated selective expansion of Vβ6/Vβ10 T cells with an approximately 3-fold preferential increase in the expansion of cytotoxic CD8⁺ T cells over CD4⁺ T cells. Besides causing expansion and upregulation of CD25, Compound 1-stimulated T cells in vitro show enhanced T cell effector cytokine production (e.g., IFN-γ and IL-2) in human and NHP primary T cells from peripheral blood mononuclear cells (PBMCs).

TABLE E1
Compound 1 Non-clinical Pharmacology Summary
with Mean EC₅₀ Values
(ND = not done, NA = not applicable):

| | | Human in vitro | | NHP in vitro | |
|---|---|---|---|---|---|
| Com- | | PBMCs | | PMBCs & in vivo | |
| pound 1 | | Mean | EC₅₀ | Mean | EC₅₀ |
| Pharma- | | EC₅₀ | range | EC₅₀ | range |
| cology | Assay | (nM) | (nM) | (nM) | (nM) |
| Non- | TCR Vβ6-5 | 1.7 | NA | 4.9 | NA |
| cellular | binding | | | | |
| target | IL-2Rα | 45 | NA | 48 | NA |
| binding | binding | | | | |
| (SPR) | IL-2Rβγ | 3.8 | NA | 3.3 | NA |
| | binding | | | | |
| Vβ T cell | In vitro Vβ | 2.82 | 0.09-10.14 | 0.13 | 0.003- |
| activation/ | total T cell | | | | 0.166 |
| expansion | expansion | | | | |
| | In vitro Vβ | 1.98 | 0.07-5.36 | ND | ND |
| | CD8⁺ T cell | | | | |
| | expansion | | | | |
| | In vitro Vβ | 5.16 | 0.07-22.2 | ND | ND |
| | CD4⁺ T cell | | | | |
| | expansion | | | | |
| | In vivo Vβ | NA | NA | 3.6 | NA |
| | CD8⁺ T cell | | | | |
| | expansion | | | | |
| IL-2 | In vitro | 0.9 | 0.52-1.87 | ND | ND |
| pathway | pSTAT5 | | | | |
| activation | staining | | | | |
| | CD4+ | | | | |
| | T cells | | | | |
| | In vitro | 26.3 | 15.9-45.8 | ND | ND |
| | pSTAT5 | | | | |
| | staining | | | | |
| | CD8+ | | | | |
| | T cells | | | | |
| In vitro | IFNγ | 1.1 | 0.00375-3.56 | ND | ND |
| cytokine | TNFα | 19.74 | 0.526-27.72 | ND | ND |
| release in | IL-6 | 12.2 | 0.28-43.65 | ND | ND |
| PBMCs | IL-1β | 23.5 | 1.56-35.82 | ND | ND |

In an ex vivo autologous human organoid model established from primary tumor tissues of four patients with microsatellite stable (MSS), high mutational burden (TMB-H) tumors, Compound 1 potently activated and expanded in situ autologous TILs that then effected potent killing of surrounding tumor tissue in 3 out of the 4 donor organoids. In the same model, addition of pembrolizumab to these organoids only led to partial tumor killing in 1 donor organoid.

### (B) In Vivo Pharmacology

Since there is limited homology between human and mouse or other rodent species (e.g., rat, rabbit) at the TRB locus, and a direct murine homolog of the Vβ6 gene (TRBV6) does not exist, the likelihood of species cross-reactivity of Compound 1 beyond monkeys is considered very low. Therefore, efficacy experiments performed in syngeneic murine tumor models were conducted with a mouse surrogate molecule (mSTAR) of the same IL-2 antibody-fusion design, that targets and expands Vβ13 TCR-expressing murine T cells and recapitulates the immunology of Compound 1 in murine T cells. The in vivo pharmacology of Compound 1 was investigated in single and repeat dose studies in cynomolgus monkeys in which the binding and potency of Compound 1 is similar to humans.

### (a) In Vivo Pharmacology—Mouse.

Dosing of the murine surrogate molecule mSTAR to tumor bearing mice was well tolerated across doses from 0.3-1.5 mg/kg when dosed IP weekly or twice weekly, with limited significant effects on body weight within expected limits (within +/−10% of total body weight). Dosing of multiple syngeneic murine solid tumor models known to have a high tumor mutational burden (TMB), mSTAR elicited when dosed weekly as monotherapy elicited potent, dose-dependent and durable anti-tumor effects, including complete regressions of established tumors. The anti-tumor activity of mSTAR was observed in multiple models that are known to be (at least partially) sensitive to other T cell activation approaches (e.g., checkpoint inhibitor [CPIs]), such as CT26. MC38 or EMT6, in addition to CPI-refractory models (e.g., B16F10, RENCA, RM-1).

Anti-tumor activity in these models was associated with increased infiltration of CD8⁺ T cells within the tumor microenvironment, in particular the accumulation of Vb13 CD8⁺ T cells. This is consistent with the proposed mechanism of action of mSTAR that likely induces expansion and infiltration of tumor-reactive Vb13 T cells from the periphery into the tumor microenvironment (TME), as well as local T cell expansion within the tumor. Of note, significant anti-tumor activity was observed in the B16F10 melanoma model known to be poorly infiltrated by immune cells, having low class 1 MHC and being refractory to CPI treatment.

Depletion of Vβ13 cells completely abolished the anti-tumor activity induced by mSTAR. These data highlight the ability of conditional Vβ13 TCR activation in context of IL-2 co-stimulation to drive expansion, activation, and anti-tumor activity in a relatively immune-depleted TME. Furthermore, upon rechallenging cured CT26 and EMT6 mice with the respective tumor cells, no tumor regrowth was observed in any mice, demonstrating induction of long-term functional immune “memory” that also appeared to be dependent on of the accumulation of memory CD8⁺Vβ13 T cells.

In an exploratory study in mice to investigate the potential for idiosyncratic IL-2-mediated vascular leak syndrome (VLS) with the murine surrogate molecule mSTAR, no signs of vascular injury or liver transaminitis were observed following administration of mSTAR at any dose level compared with aldesleukin, which caused significant VLS and transaminitis. This study suggests Compound 1 may have a lower propensity to induce vascular injury or VLS in humans compared with untargeted IL-2.

### (b) In Vivo Pharmacology—Cynomolgus Monkey

A single dose study of IV doses of 0.5, 1, and 1.5 mg/kg Compound 1 and a subsequent repeat dose study of the effects of repeated (3 consecutive) IV administrations of Compound 1 on PK, pharmacodynamics, and exploratory immunological and physiological endpoints was undertaken across 0.02, 0.1, and 0.5 mg/kg dose levels. At the highest dose level (0.5 mg/kg) two different dose schedules were investigated; QW and once every 2-weeks (Q2W) dosing over 3 consecutive doses. Overall, both single and repeat IV doses of Compound 1 were well tolerated with no signs of serious toxicity or body weight loss in either study. There were no deaths in either study.

Compound 1 was generally well tolerated and induced potent expansion of Vβ6/Vβ10 T cells (particularly CD8⁺ T cells) in blood. Analysis of target CD8⁺ Vβ6/Vβ10 T cell expansion in monkeys across a wide dose range suggests maximal pharmacological activity of Compound 1 was achieved at doses of 1 mg/kg with the dose that achieves 50% of the T cell expansion effect in monkeys (ED₅₀) estimated to be 0.029 mg/kg. Expanded monkey peripheral Vβ6/Vβ10 T cells also showed evidence of activation (e.g., upregulation of CD25) and significant IL-2R engagement and pathway activation. Moderate increases in the frequency of Vβ6/Vβ10 conventional CD4′ T cells was also observed at 0.5 mg/kg doses of Compound 1 (˜1-2-fold increase in frequency over baseline) with minimal changes in FoxP3⁺ regulatory T cells vs. baseline levels.

In repeat dose studies, particularly with QW dosing schedules at higher doses (0.5 mg/kg) consistent Vβ6/Vβ10 CD8⁺ T cell expansion was observed over consecutive three weekly dosing cycles, with sustained levels of Vβ6/Vβ10 CD8 T cells through the follow-up period, 21 days post the last dose.

In general, there were no consistent Compound 1-related effects on TNF-a, IL-1 b and IFN-g and levels of these cytokines were generally low (e.g., mean IFN-γ levels 404.7 pg/mL) in non-GLP monkey studies. Levels of serum IL-6 were only detectable in monkeys administered ≥0.5 mg/kg Compound 1 on Day 2 or 3 post dosing. In monkeys receiving 1 mg/kg IV Compound 1, generally low and variable levels of IL-6 were detectable on Day 2 or 3 post infusion (mean 697.5 pg/mL±664.8). In the repeat dose non-GLP study, dose-related increases in the chemokines MCP-1, MIP-1β and also IL-1RA were also observed. Compared to published reports of cytokine release in NHPs studies of anti-CD3 bi-specific antibodies, levels of circulating cytokines in monkeys dosed with IV Compound 1 appeared lower.

Finally, fitted dose-response relationships in Compound 1-dosed monkeys suggests significant differences in the potency of in vivo T cell expansion compared with cytokine release in monkeys. For example, the ED₅₀ for Vβ6/Vβ10 CD8⁺ cell expansion in monkeys is 0.029 mg/kg, whereas the ED₅₀ for IL-6 release is 0.18 mg/kg, further highlighting a potentially favorable therapeutic index for Compound 1.

### Compound 1 Non-Clinical Pharmacokinetics.

### (a) Single Dose Pharmacokinetics—Cynomolgus Monkey.

Single dose pharmacokinetics in monkeys were characterized from the single dose non-GLP pharmacology study in addition to the first cycle of the repeat dose study and are reported together.

TABLE E2
PK Parameters for Compound 1 Following Single Dose Infusion.

| Dose | AUC_{1 week} | AUC_{inf} | | AUC_{1 week}/Dose | AUC_{inf}/Dose | Cₘₐₓ/Dose |
|---|---|---|---|---|---|---|
| (mg/kg) | (nM*h) | (nM*h) | Cₘₐₓ (nM) | (nM*h)/(mg/kg) | (nM*h)/(mg/kg) | (nM)/(mg/kg) |
| 0.02 | 39.55 (19.97, | 40.22 (20.17, | 4.63 (3.17, | 1978 (998.3, | 2011 (1009, | 231.5 (158.5, |
| | 40.3) | 40.71) | 4.835) | 2015) | 2036) | 241.8) |
| 0.1 | 202.3 (198, | 235.3 (234.5, | 18.87 (16.15, | 2023 (1980, | 2353 (2345, | 188.7 (161.5, |
| | 212) | 236.1) | 21.34) | 2120) | 2361) | 213.4) |
| 0.5 | 1866 (1304, | 1866 (1304, | 114.7 (81.4, | 3732 (2608, | 3732 (2608, | 229.4 (162.8, |
| | 2984) | 2985) | 173.6) | 5968) | 5970) | 347.1) |
| 1.0 | 4807 (3051, | 4808 (3052, | 256.7 (147.2, | 4807 (3051, | 4808 (3052, | 256.7 (147.2, |
| | 5653) | 5655) | 272.1) | 5653) | 5655) | 272.1) |
| 1.5 | 8858 (7797, | 8859 (7798, | 297.4 (293.6, | 5905 (5198, | 5906 (5199, | 198.3 (195.7, |
| | 9920) | 9921) | 301.3) | 6613) | 6614) | 200.8) |

As shown in the table above, although the Cₘₐₓ of Compound 1 increased dose proportionally, clearance (CL) appeared to increase with lower doses of Compound 1. This non-linear pharmacokinetics suggests a saturable target-mediated effect on drug disposition (TMDD). Given the non-linear PK of Compound 1 due to TMDD, serum half-life varies with dose, with a non-specific clearance serum elimination half-life (t_{1/2}) of 1.3 days in monkeys. In the single and repeat dose studies, the nominal Tm across most dose groups was 0.25 hours (i.e., the first sampling timepoint as expected for an IV dosed antibody).

### (B) Repeat Dose Pharmacokinetics—Cynomolgus Monkeys.

Following 3 weekly doses of IV Compound 1 Cₘₐₓ was again dose-proportional over consecutive dosing administrations and was consistent over all 3 doses for the 0.5 mg/kg QW regimen. Based on first cycle (single dose) data from this study, the non-linear PK of Compound 1 was again apparent (see the table above).

Significant increases in clearance were observed over subsequent dosing cycles with lower QW doses and particularly in monkeys dosed with 0.5 mg/kg Q2W. Moreover. Compound 1 Cₘₐₓ and AUC parameters decreased following subsequent dosing cycles in all but the 0.5 mg/kg QW group. This apparent increase in clearance of Compound 1 was considered to be due to the accumulation of “clearing” anti-drug antibodies (ADA) upon repeated dosing with Compound 1, as a result of monkey anti-human antibody (MAHA) responses (Section Immunogenicity below).

### (C) Immunogenicity.

Significant ADA responses were detected in the 3-week repeat dose study in all monkeys in the 0.5 mg/kg Q2W dose group and in a single monkey in the 0.5 mg/kg QW dose cohort after the second and third doses of Compound 1 (by Day 21). Furthermore, in the 4-week GLP toxicology study, all monkeys tested positive for ADAs to Compound 1 after dosing of 4 weekly IV infusions of Compound 1 (by Day 22). The development of MAHA to fully human protein therapeutics is common in cynomolgus monkeys and does not necessarily predict the likelihood of human anti-human antibody responses in potential human trial participants.

### Compound 1 Toxicology.

No GLP single dose toxicity studies were conducted with Compound 1. In single and 3-week repeat dose non-GLP pharmacology studies, exploratory clinical observations and physiological assessments were made that are pertinent to the assessment of Compound 1 toxicology and are summarised here. There were no deaths or Compound 1-related variations in body weight in single and repeat-dose non-GLP pharmacology studies in monkeys. Hunched posture, reduced activity, and reduced appetite were noted monkeys receiving >0.5 mg/kg IV Compound 1. In general, these observations were more pronounced and consistent among monkeys dosed with ≥1 mg/kg IV Compound 1 and were accompanied by signs of liquid or soft feces, reduced activity, and mild dehydration (skin turgor). These observations typically lasted 1-2 days after dosing and resolved by Day 8 post dose, without the need for intervention in most cases. In general, most of these clinical signs were not associated with any changes in vital signs, exploratory measurements of blood pressure or ECG parameters in non-GLP pharmacology studies. However, in the non-GLP single dose monkey study, the 2 monkeys receiving the highest dose (1.5 mg/kg) of IV Compound 1 showed hunched posture, reduced appetite, diarrhea, mild dehydration, and elevated body temperature (pyrexia) beginning Day 3 post dosing that required infusion of meloxicam and subcutaneous (150-200 mL) fluids on Days 3 and 4. These animals completely recovered by Day 8. Dose-related increases in serum CRP were also noted in non-GLP pharmacology studies (CRP; 4.67-51.38 mg/dL across 0.02-1.5 mg/kg dose range) on Day 2 post infusion and minimal to mild decreases in red blood cells, hemoglobin, hematocrit, and reticulocyte counts between 4-5 days after end-of-infusion (EOI) were also observed with recovery by Day 8. These effects were all mild and transient and considered secondary to Compound 1-induced T cell activation and associated mild to moderate inflammatory response.

Finally, exploratory modified lead II ECG monitoring and telemetered blood pressure and heart rate (HR) monitoring was carried out in the 3-week repeat dose non-GLP study of IV Compound 1 in cynomolgus monkeys. In this study, there were no Compound 1-related changes in the PR, QRS, or QT corrected algorithm (QTca) interval or any hemodynamic parameters (mean arterial blood pressure, systolic blood pressure, and diastolic blood pressure) compared to the vehicle treated monkey or to pretreatment values at any dose or time point. A mild increase in HR was observed in monkeys receiving the highest dose (0.5 mg/kg) of IV Compound 1, manifesting as a lack of normal decline during the nighttime/resting period that was likely attributable to a mild, inflammation-induced sinus tachycardia that was only revealed during the nighttime/resting period.

### (A) Repeat Dose GLP Toxicology in Cynomolgus Monkeys.

A 4-week GLP toxicology study of repeat (four weekly) IV infusions of Compound 1 in 32 cynomolgus monkeys (22 receiving Compound 1) with a 4-week recovery period was conducted with a low-dose (0.1 mg/kg), a mid-dose (0.5 mg/kg), and a high-dose (1 mg/kg) of IV Compound 1. Primary necropsy was conducted at Day 29 and four additional monkeys receiving 1 mg/kg (and four corresponding control monkeys) were assigned to a recovery group with a 4-week washout period prior to necropsy on Day 57.

There was no treatment-related mortality during the course of the 4-week GLP toxicology study and no treatment-related ophthalmic findings, cardiology findings and no effect on body weight in the GLP toxicology.

There was a single unscheduled death (early euthanasia due to poor animal condition) with inconclusive findings of inflammatory gastritis and possible infectious etiology (Campylobacter). Reduced activity, reduced appetite and abnormally elevated levels of blood neutrophils and platelets before treatment. Therefore, the cause of this unscheduled death was uncertain but possibly related to a pre-morbid condition of possible infectious etiology. The possibility that Compound 1 dosing may have exacerbated this potential pre-existing condition also remains uncertain.

Hunched posture lasting 1 to 2 days was noted in 2 out of 5 male and 3 of 5 female animals in the highest dose group (1 mg/kg) in the GLP toxicology study, typically 4 to 5 days after the first dose. Hunched posture was only observed in animals administered the highest dose of 1 mg/kg IV Compound 1. Occasional instances of liquid or soft feces with reduced appetite were observed in some animals receiving 0.5 mg/kg and 1 mg/kg. These observations were not associated with changes in vital signs or ECG, required no intervention and spontaneously resolved within the recovery period.

Transient, decreases in red blood cells, reticulocyte counts and hemoglobin were noted in the GLP toxicology study that reached significance with doses ≥0.5 mg/kg IV Compound 1 and were considered related to the mild-moderate transient Compound 1-induced inflammatory effect in monkeys. Minimal to mild changes in bilirubin, albumin, and electrolytes were also noted, and marked, dose-related increases in CRP were observed 48 hours post-dosing (20-38 mg/dL across 0.1-1 mg/kg dose range). Following the 4-week recovery period (Day 57), changes noted in albumin and urea nitrogen were still minimally decreased, and CRP were minimally increased in males. Hemoglobin in males was moderately decreased at Day 57, with moderately decreased reticulocyte count.

Microscopically, perivascular mononuclear cell infiltration (minimal to mild and predominantly lymphocytes with minimal/no neutrophils or macrophages) and vascular/perivascular mononuclear cell inflammation (minimal to moderate) was observed in monkeys dosed with Compound 1. In general, the incidence and severity were greater with increasing dose level; however, individual tissues did not always demonstrate a clear dose response. This perivascular infiltrate was not associated with signs of vasculitis, vascular remodeling, vascular injury, or loss of endothelial integrity. Minimal-moderate Compound 1-related monocytic cell infiltration was also noted in other tissues and organs and at the site of administration that was in part considered related to procedural injury. There was partial resolution of perivascular mononuclear cell infiltration with complete resolution of the perivascular mononuclear cell inflammation at Day 57 and complete resolution of the mononuclear cell infiltration in the other tissues.

The mostly minimal to mild mononuclear cell infiltration and mononuclear cell inflammation described in multiple tissues, are considered to be pharmacologically-mediated and a consequence of Compound 1-induced expansion of Vb6/Vb10 T cells and enhancement of diapedesis and trafficking of Vb6/Vb10 T cells to tissues. Consistent with the basic function of T cells, Vβ6/Vβ10 CD8⁺ T cells likely trafficked to vascular sites of tissue entry where they are primed to act but do not do so until they encounter pathologic or non-self-antigen presentation. This consideration is consistent with the histopathology findings in the GLP toxicology study where there was accumulation of T cells but limited histological correlates of tissue injury.

Multi-organ lymphocytic infiltration and inflammation is a common finding in cynomolgus monkeys treated with antibodies that induce T cell activation as evidenced by pathology assessments undertaken in GLP toxicology studies for marketed CPIs such as nivolumab, atezolizumab, and ipilimumab. At 1 mg/kg in monkeys, Compound 1 elicited maximal effect of target Vβ6/Vβ10 T cell expansion and activation, with significant V 6/Vβ10 T cell expansion observed at doses of 0.02 to 0.5 mg/kg.

In the 4-week GLP toxicology study, IV Compound 1 was considered adverse at a dose level of 1 mg/kg/dose due to the multiorgan vascular/perivascular mononuclear cell inflammation on Day 29. Based on these results, the no-observed-adverse-effect level (NOAEL) was considered to be 0.5 mg/kg and the highest non-severely toxic dose (HNSTD) is considered to be 1 mg/kg IV Compound 1. At the NOAEL, the mean Compound 1 Cₘₐₓ and AUCₜₗₐₛₜ values for males were 117.8 nM and 1635.14 nM·h/mL, and for females 116.92 nM and 1556.02 nM·h/mL, respectively, following infusion of first dose of IV Compound 1 in monkeys.

### Example 2: A Phase 1/2, First-in-Human, Open-Label, Dose Escalation and Expansion Study of a Selective T Cell Receptor (TCR)-targeting, Bifunctional Antibody-fusion Molecule, in Subjects with Unresectable, Locally Advanced, or Metastatic Solid Tumors that are Antigen-rich

### Introduction

### Investigational Product, Compound 1

A bifunctional antibody-fusion molecule comprising an antibody Fab region that binds to a subset of human αβ T cells expressing the germline-encoded variable beta 6 and 10 (Vβ6/Vβ10) chains of the T cell receptor (TCR) and a physiologically active, unmodified human IL-2 molecule (the other binding arm) fused to the IgG1 antibody Fc domain containing an N297A mutation to prevent interaction with Fc receptors. Compound 1 will be administered as an intravenous (IV) infusion over 30 to 60 minutes±5 minutes (starting doses will be administered over 60 minutes±5 minutes; infusion times to be revised based on safety and pharmacokinetic (PK) data during the study).

### Study Rationale:

Preclinical studies in Example 1 described below indicate that Compound 1 represents a novel therapeutic strategy and provide a strong scientific rationale to investigate its potential effectiveness and safety in humans. Compound 1 is a first-in-class T cell activator that selectively targets subsets of T cells expressing distinct germline-encoded VP chain variant TCRs that are enriched in tumor infiltrating lymphocytes (TILs). Compound 1 potently expands naïve and antigen-specific T cells from healthy donors and cancer patients. In checkpoint inhibitor (CPI) therapy-refractory human and murine tumor models with a high tumor mutational burden, Compound 1 and its murine surrogate, mSTAR, induced potent anti-tumor activity as monotherapy, including eradicating established tumors. This anti-tumor activity was mediated by selective expansion of targeted Vb CD8⁺ memory T cells. IV infusion of Compound 1 was generally well tolerated in cynomolgus monkeys overdoses that induce significant expansion of activated Vβ6/Vβ10 CD8⁺ T cells. Taken together, these data support the potential that Compound 1 might promote both potent and durable anti-tumor responses as a single agent in humans and provide a strong scientific rationale to investigate its potential effectiveness and safety in this Phase 1/2 trial.

### Benefit Risk Assessment

This FIH study plans to enroll a study population of subjects who have unresectable, locally advanced, or metastatic solid tumors for which standard curative therapies do not exist or are no longer effective, including therapies with CPIs which often represent the limited, if not the last, options. Therefore, these subjects have a high unmet medical need.

As described in relevant sections, It has been shown that Compound 1: 1) has a novel mechanism of action in selectively activating and expanding memory T cells, generally thought to be critical for mediating and maintaining tumor regression in vivo; 2) has demonstrated ex vivo anti-tumor activities and its murine surrogate has led to eradication of established tumors and long-term maintenance of tumor-free status (cure); 3) activates T cells with a different cytokine expression profile than anti-CD3 T cell engagers (TCEs) based on published reports and thus may cause lower toxicities; and 4) has been shown to be generally well tolerated in cynomolgus monkeys. In addition, the proposed starting dose for this trial based on the minimally anticipated biological effect level (MABEL) approach is 50-fold lower than the NOAEL and 16.7-fold lower than the 1/6 of HNSTD determined from these NHP studies. NHP studies also indicated that Compound 1 had its intended pharmacological effects of activating T cells and expanding Vb6/Vb10 CD8+ T cell in vivo.

Taken together, the potential benefit provided by Compound 1, a novel T cell activator with potent anti-tumor activity demonstrated in the preclinical studies and with good tolerability in NHP studies, outweighs the potential risks in the planned study population in this FIH study.

Indication: For treatment of subjects who have histologically confirmed solid tumors that are unresectable, locally advanced, or metastatic and for which standard curative therapies do not exist or are no longer effective.

Trial population and Rationale: Subjects who have tumors with high tumor mutational burden (TMB-H) or microsatellite instability (MSI)-H/mismatch repair (MMR) deficient or virally associated (“immune hot tumors”) for both Phase 1 and 2. Subjects with “immune cold tumors” such as relapsed and refractory epithelial ovarian cancer and metastatic castration-resistant prostate cancer (mCRPC) to test whether Compound 1 could modulate the tumor immune microenvironment of these cancers and have anti-tumor activities in Phase 2.

Tumor Mutational Burden (TMB) has been shown to be a good enrichment/predictive biomarker for clinical response to checkpoint inhibitors (CPIs) and other immunotherapies, on the basis that tumors with TMB-H will likely present more neo-antigens and hence are more immunogenic for triggering a T cell response. For example, in the KEYNOTE158 study for subjects receiving pembrolizumab and assessed for the association between anti-tumor activity and TMB, objective responses were observed in 30 of 102 subjects (29%; 95% confidence interval [CI]: 21-39%) in the TMB-H group and 43 of 688 subjects (6%: 95% CI: 5-8%) in the non-TMB-H group.

Tumors with MSI are deficient in deoxyribonucleic acid (DNA) MMR and consequently have a large mutational burden with presentation of large number of neoantigens. Clinical confirmation was derived from data from subjects who were enrolled in 1 of 5 single-arm clinical trials. In 149 subjects whose tumors harbored MSI, an overall response rate (ORR) of 39.6% (95% CI: 31.7-47.9%) had been shown with responses lasting 6 months or more for 78% of those who responded to pembrolizumab. There were 11 complete responses (CR) and 48 partial responses (PR). ORR was similar irrespective of whether subjects had colorectal cancer (CRC) (36%) or a different cancer type (46% across the 14 other cancer types).

In an ex vivo human autologous TIL organoid model, the Sponsor showed that Compound 1 had a strong anti-tumor activity in the TMB-H tumors.

TABLE E3
Study Objectives and Endpoints:

| | | |
|---|---|---|
| Phase | Objectives | Endpoints |

Primary

| | | |
|---|---|---|
| Phase 1: Dose | To characterize the safety, | DLTs |
| Escalation | tolerability, and dose-limiting | Safety (including but not limited to): |
| | toxicities (DLTs), and determine | treatment-emergent adverse events |
| | the recommended Phase 2 dose | (TEAEs), serious adverse events (SAEs), |
| | (RP2D) of the investigational | deaths, clinical laboratory abnormalities |
| | product | per National Cancer Institute (NCI) |
| | | Common Terminology Criteria for |
| | | Adverse Events (CTCAE), Version 5.0, |
| | | and CRS per American Society for |
| | | Transplantation and Cellular Therapy |
| | | (ASTCT) criteria |
| Phase 2: Dose | To further explore anti-tumor | Overall response rate (ORR) defined as the |
| Expansion | activity of Compound 1 in | proportion of subjects who have complete |
| | unresectable, locally advanced, or | response (CR) + partial response (PR) per |
| | metastatic solid tumors | immune response evaluation criteria in solid |
| | | tumors (iRECIST) |

Secondary

| | | |
|---|---|---|
| Phase 1: Dose | To evaluate preliminary anti- | ORR according to iRECIST |
| Escalation | tumor activity of Compound 1 in | Duration of response (DOR) defined as |
| | unresectable, locally advanced, or | time from the date of first documented |
| | metastatic solid tumors | PR/CR until the first documentation of |
| | | confirmed disease progression per both |
| | | iRECIST and RECIST v1.1 |
| | | Disease control rate (DCR) defined as |
| | | the proportion of subjects achieving |
| | | CR+PR+ SD per both iRECIST and |
| | | RECIST v1.1 |
| | To evaluate the PK profile of | Compound 1 PK parameters following |
| | Compound 1 in plasma after | single-and repeated IV infusions as data |
| | single-and repeated IV infusions | permit (e.g., area under the curve [AUC₍₀₋ₜ₎ |
| | | and AUC_{(0-inf)}] maximum concentration |
| | | [Cₘₐₓ], time to maximum plasma |
| | | concentration [Tₘₐₓ], clearance [CL], volume |
| | | of distribution at steady state [Vss], single- |
| | | dose half-life [t_{1/2}], and repeat-dose Cₘₐₓ and |
| | | concentration reached immediately before the |
| | | next dose [C_{trough}]) |
| Phase 2: Dose | To further evaluate the anti-tumor | ORR per RECIST v1.1 |
| Expansion | activity of the investigational | DOR per both iRECIST and RECIST |
| | product | v1. |
| | | DCR per both iRECIST and RECIST |
| | | v1.1 |
| | | Progression-free survival (PFS) defined |
| | | as the time from the initial infusion of |
| | | study drug until documented disease |
| | | progression or death from any cause per |
| | | both iRECIST and RECIST v1.1 |
| | | Overall survival (OS) |
| | To evaluate the PK profile of | Compound 1 population PK parameters in |
| | Compound 1 in plasma after | expansion cohorts at the RP2D (e.g., Cₘₐₓ, |
| | single-and repeated IV infusions | AUC, CL, volume of distribution [Vd]) |
| | To further characterize the safety | Safety (including but not limited to): TEAEs, |
| | and tolerability of the | SAEs, deaths, clinical laboratory |
| | investigational product | abnormalities per NCI-CTCAE, Version 5.0, |
| | | and CRS per ASTCT criteria |
| Phase 1 and | To assess ADA formation after IV | ADA incidence and titers after single-and |
| Phase 2: Dose | single-and repeat dose | repeat-IV dosing of the investigational |
| Escalation and | administration of the | product |
| Dose Expansion | investigational product | |

Exploratory

| | | |
|---|---|---|
| Phase 1: Dose | To explore the pharmacodynamic | Cell counts and frequency of Vβ6/Vβ10 |
| Escalation | effect of Compound 1 treatment | T cell subsets in peripheral blood |
| | on expansion and biology of target | mononuclear cells (PBMCs) by flow |
| | Vβ6/Vβ10 T cell subsets in blood | cytometry |
| | and tumor samples at baseline, and | Expression levels of Vβ6/Vβ10 in T cells |
| | during and after treatment with the | and other germline Vβ TCR variants in |
| | investigational product | PBMCs and TILs in tumor tissue biopsy |
| | | samples by gene expression (e.g., using |
| | | Nanostring assays) |
| | To assess the levels of serum | Levels of serum cytokines and other soluble |
| | cytokines and their relationship to | factors (e.g., sCD25) by immunoassay |
| | pharmacodynamic, safety, | following single-and repeat-dosing of |
| | tolerability, and efficacy endpoints | Compound 1 |
| | To explore the anti-tumor activity | PFS per both iRECIST and RECIST v1.1 |
| | of the investigational product | OS |
| | | ORR, DOR, DCR, PFS per iRECIST and |
| | | RECIST, and OS in subgroups of TMB- |
| | | H, MSI-H/dMMR and virally associated |
| | | cancers, with each subgroup of subjects |
| | | combined across all dose cohorts |
| Phase 2: Dose | To explore the pharmacodynamic | Cell counts and frequency of Vβ6/Vβ10 |
| Expansion | effect of Compound I treatment | T cell subsets by flow cytometry |
| | on expansion and biology of target | Qualitative and semi-quantitative |
| | Vβ6/Vβ10 T cell subsets in blood | measurement of Vβ6/Vβ10 T cells in |
| | and tumor samples at baseline, and | tumor tissue by Multiplex IHC/IF |
| | during and after treatment with IV | Changes in gene expression profile of T |
| | the investigational product | cells isolated from PBMCs, and TILs |
| | | from tumor tissue |
| | | TCR sequencing (TCRseq) of RNA |
| | | extracted from TILs of the tumor biopsy |
| | | samples |
| | | TIL phenotyping by flow cytometry (will |
| | | be undertaken at selected sites with |
| | | experience) |
| | To explore the correlation of | Circulating ctDNA in the peripheral blood |
| | circulating cell-free tumor DNA | |
| | (ctDNA) with tumor response | |
| | To evaluate the effect of | European Organization for the Research and |
| | Compound 1 on patient-reported | Treatment of Cancer Quality of Life |
| | outcomes (PROs) in the dose | (EORTC QLQ-C30) and EuroQol |
| | expansion phase | 5 dimension 5-level (EQ-5D-5L) |
| | | questionnaires |

### Study Design

This is a two-part, open-label, first-in-human (FIH) Phase 1/2 study to determine the safety, tolerability, PK, pharmacodynamics, and preliminary anti-tumor activity of Compound 1 as a monotherapy in subjects with advanced solid tumors.

The first part (Phase 1) is a dose escalation phase using a hybrid of an accelerated titration and standard 3+3 design to determine the RP2D (i.e., maximum tolerated dose [MTD] or optimal biological dose [OBD] for Compound 1 in subjects as described below. The second part (Phase 2) is a dose expansion phase at the RP2D in 10 provisional cohorts of subjects to further assess the safety and anti-tumor activity of Compound 1. For both Phase 1 and Phase 2, subjects' tumor status will be evaluated every 8 weeks (56 days) per both iRECIST and RECIST v1.1.

### Phase 1—Dose Escalation:

Phase 1 (dose escalation) will investigate initial safety, tolerability, PK, pharmacodynamics, and preliminary anti-tumor activity of Compound 1 administered via an IV infusion once every two weeks (Q2W) in subjects whose tumors are TMB-H, MSI-H/dMMR, or virally associated. Based on the cumulative assessment of safety and Compound 1 pharmacokinetic and pharmacodynamic data, the dosing schedule may be modified (e.g., once weekly [QW] or once every 3 weeks).

Subjects must have one of the following, histologically confirmed solid tumors that are unresectable, locally advanced, or metastatic and for which standard curative therapies do not exist or are no longer effective: (1) High mutational burden solid tumors (TMB-H, per local institutional testing); (2) MSI-H/dMMR cancers (per local institutional testing); (3) Virally associated tumors including Merkel cell carcinoma, cervical, oropharyngeal, anal, penile, vaginal, and vulval cancers.

Phase 1 uses a hybrid design of an accelerated titration and a conventional 3+3 dose escalation in a total of eight provisional dose levels of Compound 1 (see the table below). Dose escalation will begin with an accelerated titration using single subject cohorts for the first two dose levels, followed by a traditional 3+3 design to minimize the number of subjects receiving doses that are less likely to be beneficial. The RP2D will be selected based on integrating safety, clinical activity, PK, and pharmacodynamic data. Phase 1 of the study will consist of a Screening Period, a Treatment Period, which includes a DLT Evaluation Period (28-day period after the first investigational product administration [Cycle 1]), and a Survival Follow up Period.

TABLE E4
Dose Levels for Phase 1 Study

| Dose | Compound 1 | Number of | |
|---|---|---|---|
| Level | Dose (mg/kg) | Subjects | Comments |
| 1 | 0.01 | 1-6 | Single-subject cohort |
| 2 | 0.02 | 1-6 | Single-subject cohort |
| 3 | 0.04 | 3-6 | 3 + 3 Design |
| 4 | 0.08 | 3-6 | |
| 5 | 0.16 | 3-6 | |
| 6 | 0.34 | 3-6 | |
| 7 | 0.68 | 3-6 | |
| 8 | 0.80 | 3-6 | |

| | |
|---|---|
| Total subjects for Phase 1: | 20-48 |

TABLE E5
Schedule of Events: Phase 1 - Dose Escalation Phase (Q2W Dosing, 28-Day Cycle)

| | | Treatment Period | | | | | | | | | | | |
|---|---|---|---|---|---|---|---|---|---|---|---|---|---|
| | | | | | | | | | | | C2 and | | Survival |
| Study | Screening | C1 | C1 | C1 | C1 | C1 | C1 | C1 | C1 | C1 | Subsequent | EoT | Follow-up |
| Assessments^{a} | Period^{b} | D 1 | D 2 | D 3 | D 4 | D 5 | D 6 | D 8 | D 15 | D 21 | Cycles | Visit | Period |

Subject Review

| | | | | | | |
|---|---|---|---|---|---|---|
| Informed | X^{c} | | | | | |
| Consent | | | | | | |
| Eligibility | X | X | | | | |
| Criteria | | | | | | |
| TMB-H | X | | | | | |
| Baseline | X | | | | | |
| Demographics | | | | | | |
| Medical | X | | | | | |
| History | | | | | | |
| Physical Exam | X | X | X | Day 1 and | X | |
| | | | | Day 15 of | | |
| | | | | each cycle^{d} | | |
| Weight and | X | Wt | | Weight only - | Wt | Weight |
| Height | | only | | Day 1 of each | only | only^{e} |
| | | | | cycle | | |
| Archival tumor | X^{f, g} | | | | | |
| Chest X-Ray | X | | | | | |
| CT and/or | X | | | X^{d} | | |
| MRI | | | | | | |

Study Drug Administration

| | | | |
|---|---|---|---|
| Compound 1 | X | X | Day 1 and |
| Administration | | | Day 15 of |
| | | | each cycle |

Safety Assessments

| Vital Signs | X | X^{h} | X | X | Day 1 of each | X |
|---|---|---|---|---|---|---|
| (BP, HR, RR, | | | | | cycle^{h} | |
| Body | | | | | | |
| Temperature) | | | | | | |
| 12-Lead ECGⁱ | X | | | | C2 Day 1 | |
| | | | | | only | |
| ECOG | X | | | | Day 1 of each | X |
| Performance | | | | | cycle | |
| Status | | | | | | |
| Adverse | | Continuous | | | | |
| Event/Safety | | | | | | |
| Monitoring^{j} | | | | | | |
| Concomitant | | Continuous | | | | |
| Medications | | | | | | |

Clinical Laboratory Assessments

| | | | | | | | |
|---|---|---|---|---|---|---|---|
| Hematology | X^{k} | X^{k} | X | X | Day 1 of each | X | X^{e} |
| | | | | | cycle | | |
| Clinical | X^{k} | X^{k} | X | X | Day 1 of each | X | X^{e} |
| Chemistry | | | | | cycle | | |
| Urine Dipstick | X^{k} | X^{k} | | | Day 1 of each | X | X^{e} |
| | | | | | cycle | | |
| HIV/HBV/HC | X | | | | | | |
| V Tests | | | | | | | |
| CMV/HLA | | | | | | | |
| typing | | | | | | | |
| Thyroid | X^{k} | X^{k} | | | Day 1 of each | X | X^{e} |
| Function | | | | | cycle | | |
| (fT4 and TSH) | | | | | | | |
| Pregnancy | | | X | | Day 1 of each | X | |
| Test^{l} | | | | | cycle | | |

PK Assessments

| | | | | | | | | | |
|---|---|---|---|---|---|---|---|---|---|
| Serial PK | X | X | X | X | X | X | X | X^{m} | X |
| (Plasma)^{m} | | | | | | | | | |

Immunogenicity Assessments

| | | | |
|---|---|---|---|
| Serum ADA | X | Xⁿ | X |

Pharmacodynamic Testing

| | | | | | |
|---|---|---|---|---|---|
| Vβ6/Vβ10 | X | X | X | | X^{o} |
| CD8⁺ T Cell^{o} | | | | | |
| Serum | X | X | | | X^{p} |
| Cytokines and | | | | | |
| sCD25^{p} | | | | | |
| Nanostring - | X | | | X | |
| TCR Diversity | | | | | |
| Panel- PBMC | | | | | |
| and Tumor | | | | | |
| Biopsy^{f,g} | | | | | |

Efficacy Assessments

| Tumor Status | X^{b} | X^{q} | |
|---|---|---|---|
| Evaluation per | Day −21 | | |
| RECIST v1.1 | to −1 | | |
| and iRECIST | | | |
| Survival Status | | | X^{f} |

Abbreviations: ADA = anti-drug-antibody; BP = blood pressure; C = cycle; CD = cluster of differentiation; CMV = cytomegalovirus; CT = computed tomography; D = day; DNA = deoxyribonucleic acid; ECG = electrocardiogram; ECOG = Eastern Cooperative Oncology Group; eCRF = electronic case report form; EOI = end of infusion; EoT = end-of-treatment; fT4 = free thyroxine; HBV = hepatitis B virus; HCV = hepatitis C virus; HIV = human immunodeficient virus; HLA = human leukocyte antigen; HR = heart rate; H-TMB = high tumor mutational burden; MRI = magnetic resonance imaging; PBMC = peripheral blood mononuclear cells; PK = pharmacokinetic(s); Q2W = once every 2 weeks; RECIST v1.1 = response evaluation criteria in solid tumors version 1.1; sCD25 = soluble CD25; SOC = standard of care; SOI = start of infusion; TCRseq = T cell receptor sequencing; TSH = thyroid stimulating hormone; Vβ6 = variable β6; Vβ10 = variable β10; wt = weight.
Footnotes:
^{a}Assessments scheduled on days of dosing should be done prior to drug administration, unless otherwise
specified. All other assessments can be done ±3 days unless otherwise specified.
^{b}Assessments must be performed within 21 days prior to first dose unless otherwise specified. CT/MRI
taken as SOC within 28 days prior to first dose may be used.
^{c}Informed consent must be signed before any study-specific assessments are performed.
^{d}Every 8 weeks (56 days) using CT/MRI scan plus tumor markers and other measures as appropriate for
the sites of disease, accompanied by physical examination.
^{e}First Survival Follow-up visit (2-month follow-up) only.
^{f}All subjects to provide an archival tumor sample if available. If not, or if such samples cannot be
retrieved, a tumor biopsy is required for select subjects at Screening (see e.g., below).
^{g}Tumor biopsy at Screening and on study (Cycle 1 Day 21) for subjects enrolled after a dose escalation cohort is identified with clear evidence of Compound 1 biological effect and in backfill subjects.
^{h}On initial (first infusion) dosing day, vital signs must be assessed at pre-dose (within 30 min prior to SOI) and at EOI, and +30 minutes after SOI and EOI. On days where vital sign time points align with PK sampling time points, vital signs should be assessed prior to PK samples being drawn. On C1D 1 and C1D 15 record vital signs including BP, HR, RR and body temperature, every 4 hours after Compound 1 infusion as inpatients for 23 hours; any signs or symptoms experienced by the subject during this time period should also be recorded.
ⁱECGs to be performed as clinically indicated.
^{j}DLT Evaluation Period (28-day period after the first investigational product administration [Cycle 1])
^{k}If completed within 72 h prior to first dose, this assessment does not need to be repeated on C1D 1.
^{l}Serum pregnancy during screening and urine pregnancy test on C1D 8 and on Day 1 of each cycle from Cycle 2 and beyond.
^{m}During Dose Escalation part, blood samples for PK analysis will be collected on Day 1 of Cycle 1 at predose, and post dose at 0.5, 1, 6, 8 h EOI, and Day 2 (24 h), Day 3 (48 h), Day 4 (72 h), Day 5 (96 h), Day 6 (120 h) and Day 8 (168 h) of Cycle 1; the same blood sampling schedule for PK analysis will be repeated at Cycle 6. For details on scheduling timepoints, windows and logistics please refer to the Lab Manual.
ⁿADA samples will be collected prior to infusion on C1, C2, C3, C5, C7 and C9, then every 3rd cycle, at EoT, and follow-up visit.
^{o}Vβ6/Vβ10 CD8+ T Cell expansion flow panel blood samples will be collected on Day 1 of Cycle 1 at predose, and post dose at 6 h EOI and Day 3 (48 h) and Day 8 (168 h); Cycles 3, 6 and 12 predose on Day 1 and postdose on Day 8 (168 h). For details on scheduling timepoints, windows and logistics please refer to the Lab Manual.
^{p}During Dose Escalation part, blood samples for serum cytokines and sCD25 will be collected on Day 1 of Cycle 1 at predose, and post dose at 0.5, 1, and 6 h EOI and Day 3(48 h) and Day 8 (168 h); Cycles 3 and 6 predose and post dose (1 h and 6 h), Day 3 (48 h) and D 8 (168 h). For details on scheduling timepoints, windows and logistics please refer to the Lab Manual.
^{q}Assessments will occur every 8 weeks (56 days).
^{r}Subjects will enter the Survival Follow-up Period after receipt of the last dose of study drug and are to be followed for at least 12 months for survival by electronic communication (e.g., email) or telephone communication every 2 months. For the first 2-month follow-up in the Survival Follow-up Period, subjects will be required to have an in-clinic visit to obtain required assessments.

### Phase 2—Dose Expansion:

Once an RP2D is determined from Phase 1, Phase 2 will be initiated to evaluate Compound 1 at this RP2D in up to 10 cohorts totaling 113 to 317 subjects who have histologically confirmed solid tumors that are unresectable, locally advanced, or metastatic and for which standard curative therapies do not exist or are no longer effective. These 10 cohorts are provisional and additional cohorts may be added based on the emerging data from the dose escalation phase [Phase 1] and preclinical studies The tumor characteristics of these 10 cohorts are described in the table below. For Cohorts 1, 3 and 4, the following subjects will be excluded: (1) subjects whose tumors are primary refractory to CPI (disease progression within 3 months of therapy); (2) subjects who have specific Grade 3 to 4 immune-related adverse events (irAEs) caused by CPI.

These 10 cohorts are provisional and additional cohorts may be added based on the emerging data from the dose escalation phase [Phase 1] and preclinical studies.

Target enrollment for each cohort will be as indicated with interim analyses after 23 subjects for Cohort 1 and after 10 subjects foreach of Cohorts 2 and 10 to allow early termination for futility based on a Simon 2-stage design. If the enrollment of given cohort is anticipated to exceed 40 subjects, a new, separate Investigational New Drug (ND) application will be submitted with anew clinical protocol for that cohort.

Each cohort will be evaluated for the primary outcome of safety and ORR, defined as the incidence of confirmed complete or partial responses (CR+PR) per iRECIST. Tumor response assessment will be performed per RECIST v1.1 and per iRECIST. Phase 2 of the study will consist of a Screening Period, Treatment Period, and Survival Follow-up Period.

TABLE E6
Dose Expansion Cohorts for Phase 2 Study

| Expansion | | Number of |
|---|---|---|
| Cohort | Tumor Type and Characteristics | Subjects |
| 1 | TMB-H (≥10 mut/Mb per central testing | 23-56 |
| | using an Food and Drug Administration | |
| | FDA]-approved assay) and | |
| | CPI experienced | |
| 2 | TMB-H (≥10 mut/Mb per central testing | 10-29 |
| | using an FDA-approved assay ) that have | |
| | not received CPI (in regions or countries | |
| | where CPI is either unavailable or | |
| | inaccessible to subjects) | |
| 3 | MSI-H/DMMR cancers (per local | 10-29 |
| | institutional testing) | |
| 4 | Virally associated tumors (including | 10-29 |
| | Merkel cell carcinoma, cervical, | |
| | oropharyngeal, anal, penile, vaginal, | |
| | and vulvar cancers) | |
| 5 | K-Ras wild-type colorectal cancer (CRC) | 10-29 |
| | and MSI-L/MMR proficient | |
| 6 | K-Ras mutant CRC | 10-29 |
| 7 | Metastatic triple-negative breast | 10-29 |
| | cancer (mTNBC) | |
| 8 | Relapsed and refractory epithelial | 10-29 |
| | ovarian cancer | |
| 9 | Metastatic castration-resistant | 10-29 |
| | prostate cancer (mCRPC) | |
| 10 | Primary stage 4 or recurrent | 10-29 |
| | non-small cell lung cancer (NSCLC) | |

| | |
|---|---|
| Total Number of Subjects | 113-317 |

TABLE E7
Schedule of Events - Phase 2: Dose Expansion Phase (Q2W Dosing, 28-Day Cycle)

| | | Treatment Period | | | | | | | | |
|---|---|---|---|---|---|---|---|---|---|---|
| | | | | | | | C2 and | | Survival | |
| Study | Screening | C1 | C1 | C1 | C1 | C1 | subsequent | | Follow-up | |
| Assessments^{a} | Period^{b} | D 1 | D 3 | D 8 | D 15 | D 21 | cycles | EoT Visit | Period | Notes |

Subject Review

| | | | | | | |
|---|---|---|---|---|---|---|
| Informed | X^{c} | | | | | |
| Consent | | | | | | |
| Eligibility | X | X | | | | |
| Criteria | | | | | | |
| TMB-H^{d} | X | | | | | |
| Baseline | X | | | | | |
| Demographics | | | | | | |
| Medical | X | | | | | |
| History | | | | | | |
| Physical Exam | X | X | X | Day 1 and Day | X | |
| | | | | 15 of each cycle^{e} | | |
| Weight and | X | Weight | | Weight only - | Weight | Weight |
| Height | | only | | Day 1 of each | only | only^{f} |
| | | | | cycle | | |
| Archived | X^{g} | | | | | |
| Tumor | | | | | | |
| Chest X-Ray^{d} | X | | | | | |
| CT and/or | X | | | X^{e} | | |
| MRI^{d} | | | | | | |

Study Drug Administration

| | | | |
|---|---|---|---|
| Compound 1 | X | X | Day 1 and 15 of |
| Administration | | | each cycle |

Safety Assessments

| Vital Signs (BP, | X | X^{h} | X^{h} | X^{h} | Day 1 of each | X^{h} |
|---|---|---|---|---|---|---|
| HR, RR, Body | | | | | cycle^{h} | |
| Temperature)^{h} | | | | | | |
| 12-Lead ECG | X | | | | C2 Day 1 only | |
| ECOG | X | X | | | Day 1 of each | X |
| Performance | | | | | cycle | |
| Status | | | | | | |
| Adverse | Continuous | | | | | |
| Event/Safety | | | | | | |
| Monitoring | | | | | | |
| Concomitant | Continuous | | | | | |
| Medications | | | | | | |

Clinical Laboratory Assessments

| | | | | | | | | |
|---|---|---|---|---|---|---|---|---|
| Hematology | X | Xⁱ | | X | X | Day 1 of each | X | X^{f} |
| | | | | | | cycle | | |
| Serum | X | Xⁱ | X | X | X | Day 1 of each | X | X^{f} |
| Chemistry | | | | | | cycle | | |
| Urine Dipstick | X | X | | | | Day 1 of each | X | X^{f} |
| | | | | | | cycle | | |
| HIV/HBV/HCV | X | | | | | | | |
| Tests | | | | | | | | |
| CMV/HLA | X | | | | | | | |
| typing | | | | | | | | |
| Thyroid | X | Xⁱ | X | X | X | Day 1 of each | X | X^{f} |
| Function | | | | | | cycle | | |
| (fT4 and TSH) | | | | | | | | |
| Pregnancy | X | | | | | Day 1 of each | | |
| Test^{j} | | | | | | cycle | | |

PK Assessments

| | | | |
|---|---|---|---|
| Sparse PK | X | X | X |
| (Plasma)^{k} | | | |

Immunogenicity Assessments

| | | | |
|---|---|---|---|
| ADA | X | X^{l} | X |

Pharmacodynamic Testing

| | | | | | |
|---|---|---|---|---|---|
| Vβ6/Vβ10 | | X | X | | X |
| CD8⁺ T Cell^{m} | | | | | |
| PBL Gene | | X | X | | X |
| Expressionⁿ | | | | | |
| Cell-free DNA^{o} | | X | | | X |
| TCR Vβ6/Vβ10 | X | | | X | |
| Expression by | | | | | |
| Multiplex | | | | | |
| IHC/IF | | | | | |
| TIL Gene | | | | | |
| Expression by | | | | | |
| Nanostring | | | | | |
| CAR-T panel | | | | | |
| TCRseq | | | | | |
| TIL | | | | | |
| Phenotyping | | | | | |
| Including Treg | | | | | |
| by Flow | | | | | |
| Cytometry | | | | | |
| Tumor Biopsy^{g} | | | | | |

Efficacy Assessments

| Tumor Status | X | X^{p} | |
|---|---|---|---|
| Evaluation per | | | |
| RECIST v1.1 | | | |
| and iRECIST | | | |
| Survival Status | | | X^{q} |

Patient-reported Outcomes

| | | | |
|---|---|---|---|
| EQ-5D-3L and | X | Day 1 C2 and | X |
| EORTC QLQ- | | C3, every 3^{rd} | |
| C30 | | cycle beginning | |
| | | with Day 1 C6 | |

Abbreviations: ADA = anti-drug-antibody; BP = blood pressure; C = cycle; CD = cluster of differentiation; CMV = cytomegalovirus; CT = computed tomography; ctDNA = circulating tumor DNA; D = day; DNA = deoxyribonucleic acid; ECG = electrocardiogram; ECOG = Eastern Cooperative Oncology Group; EOI = end of infusion; EORTC QLQ-C30 = European Organization for the Research and Treatment of Cancer Quality of Life; EoT = end-of-treatment; EQ-5D-3L = EuroQol 5-dimension 5-level; fT4 = free thyroxine; HBV = hepatitis B virus; HCV = hepatitis C virus; HIV = human immunodeficiency virus; HLA = human leukocyte antigen; iRECIST = immune Response Evaluation Criteria in Solid Tumors; HR = heart rate; MRI = magnetic resonance imaging; PBL = peripheral blood lymphocytes; PK = pharmacokinetic(s); Q2W = once every 2 weeks; RECIST = Response Evaluation Criteria in Solid Tumors; SOI = start of infusion; TIL = tumor-infiltrating lymphocytes; TMB-H = high tumor mutational burden; Treg = regulatory T cells; TSH = thyroid stimulating hormone; Vβ6 = variable β6; Vβ10 = variable β10.
Footnotes:
^{a}Assessments scheduled on days of dosing should be done prior to drug administration, unless otherwise specified. All other assessments can be done ±3 days unless otherwise specified.
^{b}Assessments must be performed within 21 days prior to first dose unless otherwise specified.
^{c}Informed consent must be signed before any study-specific assessments are performed.
^{d}If assessment done within 30 days of first dose, it may be used for screening purposes.
^{e}Every 8 weeks (56 days) using CT/MRI scan plus tumor markers and other measures as appropriate for the sites of disease, accompanied by physical examination.
^{f}First Survival Follow-up visit (2-month follow-up) only.
^{g}All subjects to provide archival tumor sample if available. If not, or if such samples cannot be retrieved within 2 weeks, a tumor biopsy is required. Tumor biopsy on study (Cycle 1 Day 21) is required.
^{h}On initial (first infusion) dosing day, vital signs must be assessed at pre-dose (within 30 min prior to SOI) and at EOI. On days where vital sign time points align with PK sampling time points, vital signs should be assessed prior to PK samples being drawn.
ⁱIf completed within 72 h prior to first dose, this assessment does not need to be repeated on C1D 1.
^{j}Serum pregnancy during screening and urine pregnancy test on C1D 8 and on Day 1 of cach cycle from Cycle 2 and beyond.
^{k}Blood samples for PK analysis will be collected on Day 1 of Cycle 1 at predose, and post dose at (1 h, 6 h) after EOI; Cycle 3 and Cycle 6 at predose and post dose (1 h). For details on scheduling timepoints, windows and logistics please refer to the Lab Manual.
^{l}All ADA samples will be collected prior to infusion on Day 1 for Cycles 3, 6 and 12.
^{m}Vβ6/Vβ10 CD8+ T Cell expansion flow panel blood samples will be collected on Day 1 of Cycle 1 at predose, and post dose Day 8 (168 h); Cycles 2, 3, 6, and 12 predose on Day 1. For details on scheduling timepoints, windows and logistics please refer to the Lab Manual.
ⁿPBL gene expression whole blood will be collected on Day 1 of Cycle 1 predose and Day 8 (168 h). For details on scheduling timepoints, windows and logistics please refer to the Lab Manual.
^{o}Cell-free DNA blood sample will be collected on Day 1 of Cycle 1 predose and on Cycle 4 Day 1. For details on scheduling timepoints, windows and logistics please refer to the Lab Manual.
^{p}Assessments will occur every 8 weeks (56 days).
^{q}Subjects will enter the Survival Follow up Period after receipt of the last dose of study drug and are to be followed for at least 12 months for survival by electronic communication (e.g., email) or telephone communication every 2 months. For the first 2-month follow-up in the Survival Follow up Period, subjects will be required to have an in-clinic visit to obtain required assessments.

### Screening Period—Phase 1 and Phase 2:

Screening will occur within 21 days of the first study drug administration. Baseline tumor status will be obtained at Screening using computed tomography (CT) and/or magnetic resonance imaging (MRI) scans plus any necessary tumor markers or other exams. If taken as part of standard of care, CT/MRI within 4 weeks (28 days) prior to first dose may be used.

### Treatment Period:

Compound 1 will be administered as an IV infusion Q2W. The dosing schedule may be revised based on safety assessments and PK evaluation. A treatment cycle is defined as 4 weeks (28 days), wherein Compound 1 is administered on Day 1 and Day 15 of the 28-day cycle.

In Phase 1, subjects will be treated as inpatients (stay in-hospital) on Day 1 and Day 15 of the first cycle for all subjects enrolled and monitored closely (vital signs and observations) for 23 hours post start of infusion (SOI). Blood samples will also be taken for PK analysis during this 23-hour period. Subjects will also be asked to stay in close proximity to the study site for up to 1 week after each of these first two doses depending on subject experience of potential delayed adverse events post infusion.

Staggering: Dosing of subjects in the Phase 1 dose escalation part will be staggered such that there is an interval of at least 5 days between initiating treatment of the first and second subject in each new dose escalation cohort.

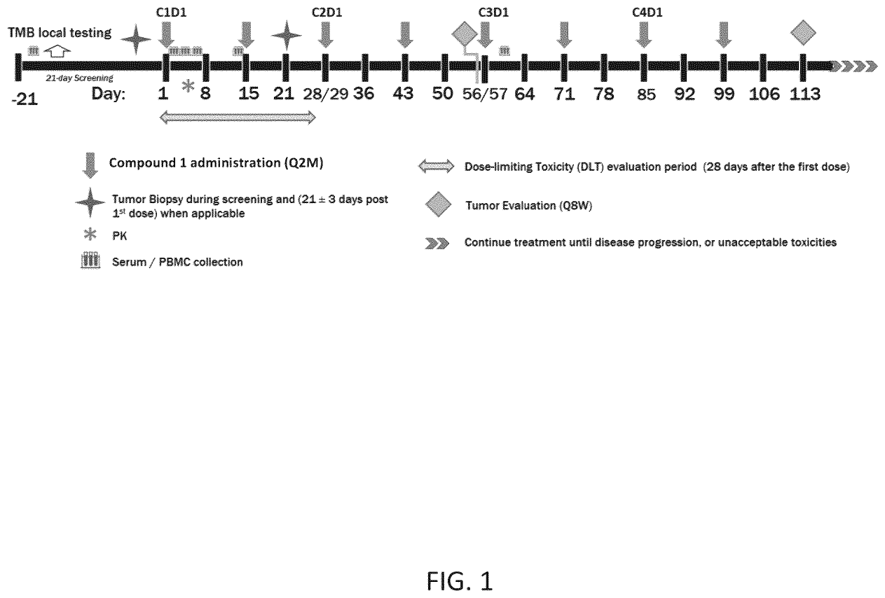

FIG. 1 is an overview of treatment and assessment plans for this study.

In Phase 2, the initial investigational product dose and dosing regimen (the RP2D) used for dose expansion will be based on the safety, tolerability, and PK evaluation in Phase 1, dose escalation.

### DLT Evaluation Period:

Following administration of the subject's initial investigational product dose in Phase 1, the subject will enter the DLT Evaluation Period which will continue up to and including the last day in the first 4-week (28-day) period (Cycle 1). Subjects who experience infusion delays for >6 consecutive days during the DLT Evaluation Period due to non-DLT or other non-drug related events (e.g., unavoidable travel delays, holidays) may remain on treatment, but will not be considered evaluable for DLTs, and will be replaced in that cohort. Subjects discontinued in the DLT Evaluation Period are to complete the End of Treatment (EoT) Visit and enter the Survival Follow-up Period. Dose escalation decisions will be supported by clinical safety and all available PK and pharmacodynamic data during the DLT Evaluation Period. Intermediate dose levels and alternative dosing schedules may be explored during Phase 1, based on review of the cumulative safety data and upon agreement with the Safety Review Committee (SRC). Any decision to implement an intermediate dose level or alternative dosing schedule will be documented and submitted to participating Institutional Review Boards (IRBs)/Independent Ethics Committees (IECs) and regulatory authorities, as appropriate, per local requirements. The MTD is defined as the highest dose level at which <33% of subjects experience a DLT during the DLT Evaluation Period. The OBD will be based on evaluation of pharmacokinetic/pharmacodynamic relationships, including but not limited to safety, the expansion of Vβ6/Vβ10 T cells, cytokine and other soluble factor levels, and clinical activity (i.e., objective tumor response). For initial dose optimization, any dose escalation cohort not exceeding the MTD can be expanded to a maximum of 10 subjects for further evaluation of safety, PK, pharmacodynamics, dosing schedule, and preliminary anti-tumor activity of Compound 1 to identify an initial optimal dose. The additional group of subjects added to a dose escalation cohort are termed backfill subjects and their data will not be used for dose escalation decision: however, their data will inform the overall safety profile and optimal dose prior to Phase 2. In Phase 2, Compound 1 dose and dosing regimen (RP2D) used for dose expansion will be based on the safety, tolerability, and PK evaluation in Phase 1, dose escalation.

### End of Treatment:

Subjects who remain clinically stable and do not experience DLTs or unacceptable toxicity during Cycle 1 (28 days) may continue treatment with Compound 1 until confirmed progressive disease (PD), initiation of alternative cancer therapy, unacceptable toxicity, withdrawal of consent, or other reasons to discontinue treatment occur. At the EoT or early discontinuation from the study, if possible, an EoT visit should be conducted.

### Survival Follow-Up Period:

After the EoT visit or 28 days after receipt of the last dose of study drug, subjects, including those discontinued during the DLT evaluation period, will enter the Survival Follow-up Period and are to be followed for at least 12 months for survival by electronic communication (e.g., email) or telephone communication every 2 months. For the first 2-month follow-up in the Survival Follow-up Period, subjects will be required to have an in-clinic visit to obtain required assessments.

Rules for subject discontinuation: The investigator may discontinue treating a subject with study drug or withdraw the subject from the study at any time for safety or administrative reasons. The subject may decide to discontinue study drug or withdraw from the study at any time for any reason. The reason for discontinuation will be documented. If a subject discontinues study drug, the subject will enter the Follow-Up Period and complete protocol-specified off-treatment visits, procedures, and survival follow-up unless the subject withdraws consent. An Investigator may discontinue a subject from the study drug for these primary reasons: a protocol violation occurs; AE(s)/SAE(s); pregnancy: occurrence of a drug-related DLT; drug interruption for ≥21 days; lost to follow-up; the Sponsor or Investigator terminates the study; withdrawal of consent by subject or proxy: other. Subjects who are confirmed to have disease progression during the study but otherwise are achieving clinically meaningful benefit may choose to continue the treatment at the discretion of the Investigator, if clinically stable defined as: absence of symptoms and signs indicating clinically significant progression of disease: no decline in Eastern Cooperative Oncology Group (ECOG) performance status; absence of symptomatic rapid disease progression requiring urgent medical intervention (e.g., symptomatic pleural effusion, spinal cord compression). Subjects will be required to reconsent to continue treatment. If a subject withdraws consent, the investigator will explain to the subject involved that the study will be discontinued, no additional study drug will be given to the subject, and will provide appropriate medical treatment and other necessary measures for the subject. If a subject is withdrawn from the study or from Compound 1 dosing for any reason other than disease progression, the Sponsor must be alerted within 24 hours.

### Starting Dose Determination

### In Vitro and In Vivo Pharmacology Determinants of MABEL

A summary of relevant non-clinical in vitro and in vivo EC50s for Compound 1 is presented in Example 1 above. The most potent effect of Compound 1 in physiologically relevant systems is the expansion of Vβ6/Vβ10 CD8+ T cells as measured by flow cytometry (EC₅₀=1.98 nM). Based on dose and exposure-response relationships fitted from Vβ6/Vβ10 CD8+ T cell expansion data from both non-GLP and GLP studies of IV Compound 1 in 41 monkeys, the plasma investigational product C., at a dose that achieves 50% of the peak Vβ6NP10 CD8+ T cell expansion in blood (i.e., the ED50) can be used to infer the “in vivo EC50” for Vβ6/Vβ10 CD8+ T cell expansion in monkeys. This monkey in vivo EC50 (3.6 nM) fits reasonably well with the human in vitro EC50 (1.98 nM) and supports the use of both human and monkey T cell expansion data to establish MABEL doses in humans.

The release of the effector lymphokine IFN-γ from human PBMCs stimulated with Compound 1 in vitro (EC50 1.1 nM) is also reflective of Vβ6/Vβ10 CD8+ T cell expansion and likely an important component of the anti-tumor activity of Compound 1. However, static in vitro systems do not recapitulate the complex binding and elimination routes present in whole organisms, and therefore likely overpredict steady-state cytokine concentrations in the blood of humans. This is somewhat confirmed by the generally low levels of IFN-γ observed in the serum of monkeys dosed with ≥mg/kg IV Compound 1. In general, the release of pro-inflammatory cytokines such as TNF-α. IL-6, and IL-1β that are postulated to mediate cytokine release syndrome (CRS) in humans, occur at higher concentrations of Compound 1 both in human in vitro systems, but also in the blood of monkeys dosed with IV Compound 1 (in vitro human and in vivo monkey EC50 range of 16-68 nM). Finally, the higher potency of Compound 1-induced phosphorylation of STAT5 (a proximal signaling molecule common to all IL-2 receptors) in human CD4+ T cells vs. CD8+ T cells is reflective of the enhanced binding of Compound 1 to CD4+ regulatory T cells (Treg) that express high levels of the high-affinity IL-2 receptor complex. However. Compound 1 in vivo induces preferential activation and expansion Vβ6/Vβ10 CD8+ T cells compared with CD4+ T cells and Treg therefore, the Sponsor proposes the in vitro CD4+ T cell pSTAT5 assay is of limited relevance in establishing MABEL in humans.

### Modeled Estimates of Human MABEL Doses

To further support the determination of an appropriate starting dose of IV Compound 1 in humans, a computational model of STAR602 human PK/pharmacodynamics was developed based on PK and pharmacodynamic data generated in 41 cynomolgus monkeys dosed with IV Compound 1 with detailed assessments of Vβ6/Vβ10 CD8+ T cell expansion (and other biomarkers) over time using the same T cell flow cytometry assay to be deployed into the proposed Phase 1/2 trial in humans. This model has been used to simulate both human Compound 1 PK and the kinetics of Vβ6/Vβ10 CD8+ T cell expansion, at various doses of Compound 1 to establish a safe starting dose and escalation schedule. Compared with single point estimates of pharmacological effect from human in vitro assays, the modelled kinetics of human T cell expansion provide a richer assessment of Compound 1 pharmacology in humans overtime. Moreover, the human PK/pharmacodynamic model also considers the effect of inter-subject variability of Compound 1 PK and pharmacodynamics.

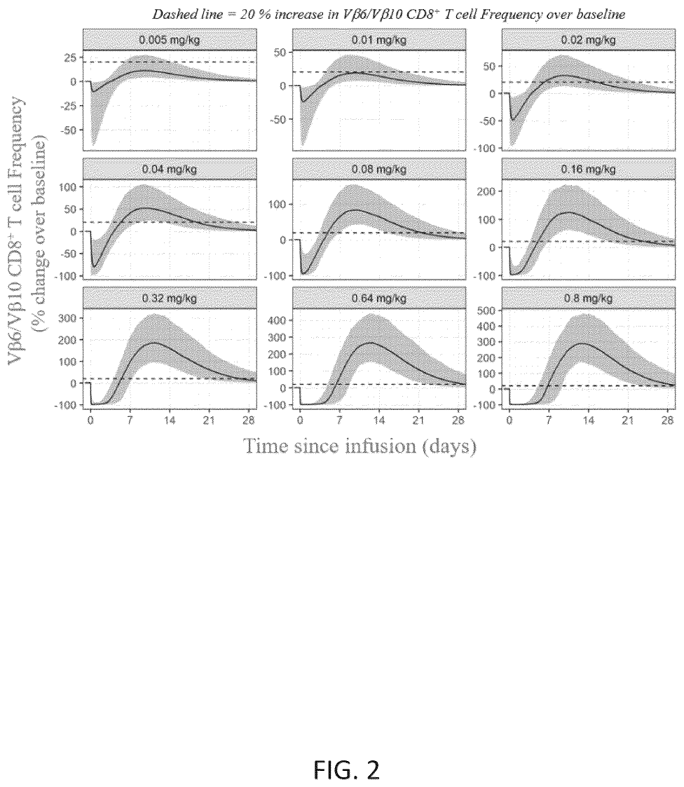

FIG. 2 shows the expected kinetics of Vβ6/Vβ10 CD8+ T cell expansion in the blood of human subjects over time following infusion of various starting doses of IV Compound 1. Using these modelled simulations, it is possible to establish the population-based pharmacodynamic effects of IV Compound 1 with reference to a target MABEL effect of 20% peak increase over baseline in Vβ6NP10 CD8+ T cell frequency. At a starting dose of 0.01 mg/kg IV Compound 1, a 20% increase in Vβ6/Vβ10 CD8+ T cells (e.g., an increase in expected baseline frequency of 6.82% to 8.18% after treatment) is achieved in a typical human subject (solid line/median effect). However, as is apparent from the shaded area, the inter-subject variability around this typical/median effect is such that significantly lower levels of T cell expansion are expected in a large proportion of subjects. Lower starting doses (e.g., 0.005 mg/kg) are predicted to achieve a lower expansion effect (˜10% Vβ6/Vβ10 CD8+ T cell increase over baseline) in a typical human with most patients not achieving the 20% MABEL target effect. In contrast, at a starting dose of 0.04 mg/kg IV Compound 1, a ≥20% increase in T cell expansion is predicted in almost all (>95%) patients with a median predicted increase over baseline of 58% (e.g., an increase from 6.82% at baseline to 10.77% after treatment).

Justification for Starting Dose of 0.01 mg/kg IV Compound 1

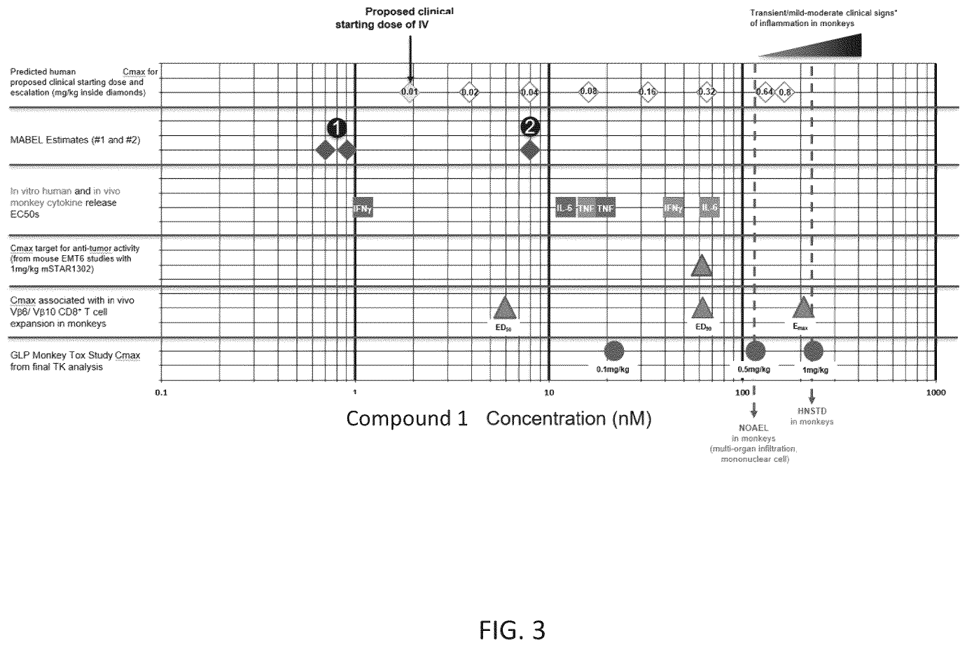

Integrating relevant Compound 1 in vitro human pharmacology parameters with fitted dose-response relationships in monkeys and predicted human Compound 1 T cell expansion allows a more complete assessment of safe starting dose. As illustrated in FIG. 3, an integrated analysis of Compound 1 pharmacology and modeled predictions highlights two investigational product MABEL estimates: (1) A MABEL based on a minimal threshold (20% of maximal effect (EC₂₀)) of the most potent pharmacological effect i.e., Vβ6NP10 CD8⁺ cell expansion. The human in vitro EC₂₀ and monkey in vivo EC₂₀ for Vβ6NP10 CD8⁺ cell expansion is 0.7 nM and 0.91 nM, respectively. Based on this concentration target, a human dose of 0.005 mg/kg IV Compound 1 is predicted to achieve a Cₘₐₓ, of 0.93 nM. (2) A MABEL that considers the modelled population variability in T cell expansion in humans to identify a dose and blood concentration of Compound 1 at which a minimum 20% increase in T cell expansion is achieved in 95% of patients. Based on this target, a dose of 0.04 mg/kg IV Compound 1 is predicted to achieve ≥20% peak increase in Vβ6NP10 CD8⁺ cell expansion in the blood of most trial subjects and is associated with a predicted Compound 1 Cₘₐₓ of 7.98 nM in humans.

Although MABEL #1 provides a point estimate of Compound 1 pharmacological effect at a lower median effect size (i.e., 20% of maximal T cell expansion response; EC₂₀), this dose is based on a static in vitro pharmacology assay that does not consider likely in vivo PK and pharmacodynamic variability in humans. In contrast, the use of modelled T cell expansion kinetics in humans provides a more physiologically relevant assessment of expected Compound 1 pharmacology in humans over time, while also factoring in the pharmacological and physiological factors that underlie the variability in the primary pharmacological effect of Compound 1 (Vβ6/Vβ10 CD8⁺ T cell expansion) in humans.

Based on these human simulations, a dose of 0.005 mg/kg IV Compound 1 is predicted to induce approximately 10% peak increase in Vβ6NP10 CD8− T cell expansion over baseline in a typical human subject, with a significantly lower effect in approximately 50% of subjects (FIG. 2). Furthermore, based on in vivo Vβ6/Vβ10 CD8⁺ cell expansion data in monkeys dosed across 0.02-1.5 mg/kg IV Compound 1 using the same flow cytometry assay as planned for the proposed clinical trial of Compound 1, it is likely that T cell expansion at a dose of 0.005 mg/kg IV Compound 1 could be largely undetectable in most subjects since robustly measurable increases in T cell expansion in monkeys were observed at ≥20% increases over baseline. In contrast, at a dose of 0.04 mg/kg, ≥20% increase in T cell expansion would be robustly detectable in most trial subjects while also resulting in a Cₘₐₓ that is approximately 1.6-8-fold lower than the in vitro human and in vivo monkey EC₅₀ concentrations for pro-inflammatory cytokine release (See Example 1 and FIG. 3).

While ensuring the starting dose of Compound 1 is set at a safe (low) effect level, a reasonable MABEL dose should elicit a measurable biological effect in the context of the anticipated kinetics of the response over time, and also the likely variability in PK and pharmacodynamic response in the human population. This is particularly relevant for the proposed patient population for the trial and the need to avoid administering sub-MABEL doses of Compound 1 that are unlikely to offer any potential for benefit in patients with high unmet medical need.

Taking these considerations into account and balancing the two approaches to the determination of Compound 1 MABEL discussed above, the proposed starting dose of IV Compound 1 for the planned Phase 1/2 trial in patients with advanced metastatic cancer is 0.01 mg/kg. This dose is predicted to achieve a Cₘₐₓ in humans of 1.91 nM with a median increase of ˜20% Vβ6/Vβ10 CD8+ T cell frequency over baseline. This starting dose of 0.01 mg/kg achieves a Cₘₐₓ that falls within the lower quartile of the range between the two MABEL estimates (0.7-7.98 nM) and thus balances a safe starting dose with the need to test pharmacologically relevant and pharmacodynamically detectable doses of Compound 1 in patients with advanced cancer. Acknowledging the possibility that T cell expansion may not be robustly detectable at the proposed starting dose of 0.01 mg/kg, an accelerated titration phase is proposed in the Phase 1 design wherein 0.01 mg/kg and 0.02 mg/kg doses will be assessed in single subjects with appropriate staggering and safety monitoring, before escalating to a 3+3 cohort at 0.04 mg/kg where the minimal ≥20% increase in Vβ6/Vβ10 CD8+ T cell frequency is anticipated to be robustly detectable in most trial subjects.

The safety of the proposed starting dose 0.01 mg/kg and the escalation scheme is considered in the context of findings and limits defined in the 4-week GLP toxicology study of IV Compound 1 in cynomolgus monkeys. The following two tables provide dose and exposure margins for the proposed Compound 1 starting dose and dose escalation scheme based on predicted human investigational product PK and proposed doses vs. toxicokinetic parameters from the first dose (cycle) of the 4-week GLP toxicology study.

TABLE E8
Safety Margin Comparisons for Starting Dose Versus GLP
Toxicology Dose Limits.

| | | Fold cover | Fold cover | Fold cover |
|---|---|---|---|---|
| Dose | Fold cover | over 1/6^{th} | over | over 1/10^{th} |
| (mg/kg) | over HNSTD | HNSTD | NOAEL | NOAEL |
| 0.01 | 100-fold | 16-fold | 50-fold | 5-fold |
| 0.02 | 50-fold | 8-fold | 25-fold | 2.5-fold |
| 0.04 | 25-fold | 4-fold | 12.5-fold | 1.25-fold |
| 0.08 | 12.5-fold | 2-fold | 6.25-fold | — |
| 0.16 | 6.25-fold | Parity | 3.12-fold | — |
| 0.34 | 2.94-fold | — | 1.5-fold | — |
| 0.68 | 1.5-fold | — | — | — |
| 0.8 | 1.25-fold | — | — | — |

HNSTD-1 mg/kg IV Compound 1. 1/6^{th} IV Compound 1-0.16 mg/kg IV Compound 1. NOAEL-0.5 mg/kg IV Compound 1 (minimal to moderate perivascular and multi-organ lymphocytic inflammation considered adverse at 1 mg/kg IV Compound 1). 1/10^{th} NOAEL-0.05 mg/kg IV Compound 1. HNSTD = highest non-severely toxic dose. NOAEL = no-observed-adverse-effect level.

Calculated Compound 1 exposure margins using Compound 1 plasma toxicokinetics in monkeys from the 4-week GLP toxicology study and predicted human plasma Compound 1 exposures across the proposed Phase 1 dose escalation are summarized in the following table.

The predicted human Compound 1 Cₘₐₓ (1.91 nM) and AUC_{0-inf} (10.52 nM·h) associated with a starting dose of 0.01 mg/kg is approximately 121-fold and 318-fold lower than the Cₘₐₓ and AUCtₗₐₛₜ measured in monkeys at the HNSTD of 1 mg/kg. Furthermore, the predicted human Cₘₐₓ and AUC_{0-inf} at a starting dose of 0.01 mg/kg is approximately 61-fold and 151-fold lower than the Cₘₐₓ, and AUCt^{last} associated with the NOAEL of 0.5 mg/kg from the 4-week GLP toxicology study in monkeys (see below).

TABLE E9
Safety Margin Comparison of Predicted Human Compound 1 Exposures Versus
Monkey Toxicokinetics from 4-week GLP Toxicology Study of IV Compound 1.

| | Human Modeled Compound 1 Predictions | | | Exposure Margins vs. | | Exposure Margins vs. | |
|---|---|---|---|---|---|---|---|
| | | | Median predicted | Monkey TK at HNSTD | | Monkey TK at NOAEL | |
| | | | Vβ CD8⁺ T cell | (1 mg/kg) | | (0.5 mg/kg) | |
| Dose | | AUC_{inf} | expansion (% | Cₘₐₓ | AUC | Cₘₐₓ | AUC |
| (mg/kg) | Cₘₐₓ (nM) | (nM · h) | increase over BL) | cover | cover | cover | cover |
| 0.01 | 1.91 | 10.52 | 19.19 | 121.89-fold | 318.93-fold | 61.21-fold | 151.25-fold |
| 0.02 | 3.91 | 28.72 | 34.66 | 59.54-fold | 116.82-fold | 29.90-fold | 55.40-fold |
| 0.04 | 7.98 | 85.86 | 58.03 | 29.17-fold | 39.07-fold | 14.65-fold | 18.53-fold |
| 0.08 | 16.20 | 268.29 | 89.36 | 14.37-fold | 12.5-fold | 7.21-fold | 5.93-fold |
| 0.16 | 32.72 | 825.83 | 136.16 | 7.11-fold | 4.06-fold | 3.57-fold | 1.92-fold |
| 0.32 | 65.80 | 2,387.39 | 202.92 | 3.53-fold | 1.40-fold | 1.77-fold | 0.66-fold |
| 0.64 | 132.00 | 6,337.68 | 289.61 | 1.76-fold | 0.52-fold | 0.88-fold | 0.25-fold |
| 0.8 | 165.10 | 8,520.82 | 321.19 | 1.41-fold | 0.39-fold | 0.70-fold | 0.18-fold |

Cₘₐₓ and AUCₜₗₐₛₜ at the HNSTD (1 mg/kg) in the 4-week monkey GLP toxicology study = 232.82 nM and 3355.23 nM · h, respectively. Cₘₐₓ and AUCₜₗₐₛₜ at the NOAEL (0.5 mg/kg) in the 4-week monkey GLP toxicology study = 116.92 nM and 1591.18 nM · h, respectively. 1 mg/kg (HNSTD) GLP toxicology TK parameters are adjusted values derived from TK data with “non-reportable values” excluded.

### Therapeutic Dose Range and IV Compound 1 Dose Escalation Strategy

Optimal anti-tumor activity in murine solid tumor models was observed at weekly (QW) or less frequent schedules at doses of 1 mg/kg of mSTAR, a murine surrogate molecule for Compound 1. From pharmacokinetic measurements of mSTAR in mice at 1 mg/kg, an exposure (AUC) target of 1731.6 nM*h and Cₘₐₓ of 64.3 nM can be derived as target plasma concentrations for efficacy in humans. Using the human PK/pharmacodynamic model, a human dose of 0.26 mg/kg IV Compound 1 is predicted to achieve this murine AUC exposure target, and a dose of 0.3 mg/kg IV Compound 1 is predicted to achieve the murine Cm target (reflected in FIG. 3).

Similarly, from pharmacodynamic assessments of Vβ CD8+ T cell expansion made in the blood of tumor-bearing mice and healthy monkeys, peripheral T cell expansion targets can be considered into the assessment of human Compound 1 therapeutic dose range. For example, the human dose of IV Compound 1 predicted to achieve the same increase in peripheral Vs CD8+ T cell frequency (˜82% increase over baseline in mice dosed with 1 mg/kg mSTAR) is 0.07 mg/kg. Finally, the human IV Compound 1 dose predicted to achieve the same increase in Vβ6/Vβ10 CD8+ T cell frequency as measured in monkeys dosed with 1 mg/kg IV Compound 1 (approximate Emax associated with ˜310% increase in Vβ6NP10 CD8+ T cells over baseline) is 0.8 mg/kg.

Considering these PK and pharmacodynamic targets from monkey and tumor-bearing mouse studies and the minor differences in potency between the murine surrogate mSTAR and Compound 1 molecules, a target therapeutically effective dose range in humans of 0.3-0.8 mg/kg is proposed.

As illustrated in the table above, the predicted human Cₘₐₓ at 0.3 mg/kg IV Compound 1 is still more than 3-fold lower than the Cₘₐₓ at the HNSTD (1 mg/kg) established in the 4-week GLP toxicology study. Escalation to a highest human dose of 0.8 mg/kg is planned in the proposed Phase 1 dose escalation, which would result in a slightly lower (1.4-fold) Cₘₐₓ than measured at the highest dose in monkeys in the GLP toxicology study (1 mg/kg; the HNSTD). However, based on the conservative assessment of CL incorporated into the human PK model, exposure at a human dose of 0.8 mg/kg (AUC_{0-inf}; 8,520.82 nM·h) is predicted to exceed exposure levels measured at the HNSTD in monkeys (AUCₜₗₐₛₜ; 3355.23 nM·h). Given the lack of severe toxicity observed in monkeys dosed with repeated 1 mg/kg infusions of Compound 1 in the GLP toxicology study, and the careful dose escalation design and safety monitoring plan proposed for the clinical trial, the Sponsor proposes cautious escalation to a highest dose of 0.8 mg/kg in the Phase 1 dose escalation, unless dose-limiting toxicities (DLTs) or the optimal biological dose (OBD) is reached earlier in the escalation.

Modest dose increments are proposed for the Phase 1 dose escalation scheme due to the immune activation potential of Compound 1. However, in contrast to anti-CD3 T cell engagers (TCEs) that induce rapid and robust post-dose cytokine release characterized by high serum levels of IL-6 and TNF-α in both cynomolgus monkeys and humans, dosing of IV Compound 1 in monkeys was associated with only mild to moderate release of IL-6 at high dose levels (≥0.5 mg/kg) 2-3 days post infusion. In published reports of tumor-targeted anti-CD3 bi-specific antibodies in monkeys, peak levels of circulating IL-6 are ˜3-5-fold higher than peak levels of IL-6 observed in monkeys dosed with 1 mg/kg IV Compound 1. Moreover, monkeys dosed with IV Compound 1 showed only mild to moderate and transient clinical signs of inflammation (hunched posture, reduced activity, reduced appetite) with no impact on other vital signs, hemodynamics, or evidence of neurotoxicity. Nevertheless, the proposed dose escalation scheme follows cautious 2-fold dose increments through to the last dose escalation to 0.8 mg/kg wherein a smaller 1.3-fold increase is proposed.

### Dosing Schedule

IV Compound 1 will be administered every two weeks (Q2W) within a 28-day cycle. In murine dose regimen-finding studies, once-weekly (QW) dosing was found to be more efficacious than more frequent dosing schedules (e.g., dosing twice per week). In non-GLP studies of IV Compound 1 in cynomolgus monkeys, repeat dosing (up to 3 doses) was associated with prolonged Vβ6/Vβ10 CD8+ T cell expansion over subsequent dosing cycles. However, when comparing the pharmacodynamic profiles of repeated QW infusions vs. Q2W schedules of Compound 1, a significant increase in the AUC of Vβ6NP10 CD8+ T cell expansion in blood was observed in monkeys dosed with Q2W Compound 1. Given the known kinetics of T cell activation and proliferation, it is possible that T cells benefit from a “rest” between cycles of activation and expansion. Therefore, a Q2W dosing schedule is proposed for the Phase 1/2 trial of IV Compound 1. The need for investigation of additional dosing schedules or modification of the proposed Q2W schedule will be considered as safety, tolerability, PK and pharmacodynamic data become available from the Phase 1 part of the study.

### Study Population Selection

Study Population: Subjects must have histologically confirmed solid tumors that are unresectable, locally advanced, or metastatic and for which standard curative therapies do not exist or are no longer effective.

### Inclusion Criteria:

Each subject must also meet all the following criteria to be enrolled in this study:

General: (1) Age ≥18 years old: (2) Eastern Cooperative Oncology Group (ECOG) performance status of ≤2; (3) Ability to provide written informed consent prior to initiation of any study-related tests or procedures that are not part of standard of care for the subject's disease. Subjects must also be willing and able to comply with study procedures, including the acquisition of specified research specimens. (4) Life expectancy ≥12 weeks. (5) Measurable disease as per RECIST v1.1 criteria and documented by CT and/or MRI. Cutaneous or subcutaneous lesions must be measurable by calipers. Lesions to be used as measurable disease for the purpose of response assessment must either: a) not reside in a field that has been subjected to prior radiotherapy; or b) have demonstrated clear evidence of radiographic progression since the completion of prior radiotherapy and prior to study enrollment.

### Tumor Histology Types:

(6) For Phase 1, subjects must have one of the following solid tumors:
a. High mutational burden (TMB-H per local institutional testing);
b. MSI-H/dMMR cancer (per local institutional testing);
c. Virally associated tumors including Merkel cell carcinoma, cervical, oropharyngeal, anal, penile, vaginal, and vulvar cancers. No viral testing required for Merkel cell carcinoma, cervical, or anal cancers. For oropharyngeal, penile, vaginal, and vulvar cancers, human papilloma virus (HPV) positivity documented per local institutional testing is required.

(7) For Phase 2, subjects must have one of the following solid tumors:
a. High mutational burden (TMB-H≥10 mut/Mb per central testing using an FDA-approved test):
b. MSI-H/dMMR cancer (per local institutional testing);
c. Virally associated tumors including Merkel cell carcinoma, cervical, oropharyngeal, anal, penile, vaginal, and vulvar cancers. No viral testing required for Merkel cell carcinoma, cervical or anal cancers. For oropharyngeal, penile, vaginal, and vulvar cancers, HPV positivity documented per local institutional testing is required:
d. Metastatic triple-negative breast cancer (mTNBC);
e. Relapsed and refractory epithelial ovarian cancer;
f. Metastatic castration-resistant prostate cancer (mCRPC);
g. K-Ras wild type CRC;
h. K-Ras mutant CRC;
i. Primary stage IV or recurrent non-small cell lung cancer (NSCLC).
   (Other histologies may also be included in Phase 2 as additional data emerge to support their inclusion.)

### Tumor Biopsy:

(8) Subjects must allow for acquisition of existing formalin-fixed paraffin-embedded archival tumor sample, either a block or unstained slides that had been procured within 3 months prior to enrollment, if available.

(9) If a study site cannot retrieve such existing archival tumor sample within 2 weeks after the initial attempt, or archival tumor is not available, the following subjects must consent to fresh baseline tumor biopsy during Screening:
Dose Escalation (Phase 1) subjects: only after a dose escalation cohort is identified with clear evidence of Compound 1 biological effect (e.g., expansion of target T cell subsets, anti-tumor effect, or other relevant biomarker assessments), i.e., subjects still enrolling in this dose escalation cohort and at the immediate next higher dose level:
Backfill subjects (in Dose Escalation): the additional subjects enrolled in Dose Escalation at a dose level deemed below or at the MTD;
Dose Expansion Cohort (Phase 2) subjects.

(10) Above subjects must also consent to a second tumor biopsy 21 days (+3 days) after the first dose administration of Compound 1.

### Laboratory Features:

(11) Acceptable laboratory parameters as follows:
Albumin ≥3.0 g/dL;
Platelet count ≥75-103/μL;
Hemoglobin ≥8.5 g/dL:
Absolute neutrophil count ≥1.5×103/μL:
ALT/AST≤3.0×upper limit of normal (ULN); for subjects with hepatic metastases, ALT and AST≤5×ULN:
Total bilirubin ≤1.5×ULN, except subjects with Gilbert's syndrome, who may be enrolled if the conjugated bilirubin is within normal limits:
Calculated creatinine clearance ≥50 mL/min based on Cockcroft-Gault formula.

### Reproductive Features:

(12) Female subjects of childbearing potential (not surgically sterilized and between menarche and 1-year post-menopause) must have a negative serum (screening) or urine pregnancy test performed within 72 hours prior to the initiation of study drug administration. Female subjects of childbearing potential must be willing to use two forms of contraception throughout the study, from the time of consent through 60 days after the last dose of Compound 1. Examples of effective contraception are birth control pills, birth control patch (e.g., Ortho Evra), NuvaRing, intrauterine device, female condom, diaphragm with spermicide, cervical cap, use of a condom by the sexual partner, documented sterile sexual partner or documented evidence of surgical sterilization (e.g., tubal ligation or hysterectomy) at least 6 months prior to Screening. Abstinence is acceptable if this is the established and the preferred contraception method for the subject. It is not necessary to use any other method of contraception when complete abstinence is elected.

(13) Male subjects with partners of childbearing potential must use barrier contraception from the time of consent through 60 days after last dose of Compound 1 and must not donate sperm during this period. In addition, male subjects should also have their partners use contraception (as documented for female subjects) for the same period of time.

### CNS Metastases:

(14) Symptomatic central nervous system (CNS) metastases must have been treated, be asymptomatic for ≥14 days, and meet the following at the time of enrollment:
No concurrent treatment for CNS disease (e.g., surgery, radiation, corticosteroids >10 mg prednisone/day or equivalent);
No concurrent leptomeningeal disease or cord compression.

### Previous Checkpoint Inhibitor Therapy:

(15) Subjects who have previously received a CPI (e.g., anti-PD-L1, anti-PD-1, anti-CTLA-4) prior to enrollment must have CPI immune-related toxicity resolved to either Grade 1 or baseline (prior to the CPI) to be eligible for enrollment.
Subjects who experienced previous CPI-related endocrine abnormalities are eligible for the study regardless of CTCAE grade if well controlled on replacement therapy.
Subjects who have had previous CPI-related Grade 3 to 4 pneumonitis, peri/myocarditis, colitis and bowel perforation, myositis, encephalitis, or peripheral neuropathy should be excluded.

### Exclusion Criteria:

Subjects who meet any of the following criteria will be excluded from the study:

(1) Subjects with a history of known autoimmune disease with exceptions of: (a) Vitiligo: (b) Psoriasis, atopic dermatitis, or other autoimmune skin condition not requiring systemic treatment: (c) History of Graves' disease, now euthyroid for >4 weeks; (d) Hypothyroidism managed by thyroid replacement; (e) Alopecia; (f) Arthritis managed without systemic therapy beyond oral nonsteroidal anti-inflammatory drugs; (g) Adrenal insufficiency-well controlled on replacement therapy.

(2) Major surgery or traumatic injury within 8 weeks before first dose of study drug.

(3) Unhealed wounds from surgery or injury.

(4) Treatment with >10 mg per day of prednisone (or equivalent) or other immune-suppressive drugs within 7 days prior to the initiation of study drug. Exceptions may be made for patients who have had allergic reaction to iodinated contrast media. Steroids for topical, ophthalmic, inhaled, or nasal administration are allowed.

(5) Prior therapy within the following timeframe before the planned infusion of Compound 1 as follows:
Cytotoxic chemotherapy, small molecule inhibitors, radiation, interventional radiology procedure, or similar investigational therapies within ≤2 weeks or subjects who have not recovered (i.e., ≤Grade 1 or to baseline) from AEs due to a previously administered agent;
Monoclonal antibodies, antibody-drug conjugates, radioimmunoconjugates, or similar investigational therapies within 6 weeks prior to the initiation of study drug or subjects who have not recovered (i.e., ≤Grade 1 or to baseline) from AEs due to agents administered more than 6 weeks earlier. Note: Subjects with ≤Grade 2 neuropathy or ≤Grade 2 alopecia are an exception to this criterion and may qualify for the study.
   Note: Concurrent use of hormones either to maintain castrate levels of testosterone in subjects with castration-sensitive prostate cancer or for non-cancer-related conditions (e.g., insulin for diabetes, hormone replacement therapy) is acceptable. Bisphosphonates are permitted for supportive care of bone metastases (e.g., breast or prostate cancer).

(6) Clinically significant cardiovascular/vascular disease including:
Myocardial infarction or unstable angina <6 months prior to the initiation of study drug;
Clinically significant cardiac arrhythmia (e.g., with potential for hemodynamic instability) not controlled or unresponsive to treatment;
Uncontrolled hypertension: systolic blood pressure >180 mmHg and/or diastolic blood pressure >100 mmHg;
Pulmonary embolism, stroke, or transient ischemic attack <6 months prior to initiation of Compound 1:
QTcF (Fridericia correction) prolongation >480 msec:
Congestive heart failure (New York Heart Association Class III-IV);
Pericarditis/clinically significant pericardial effusion:
Myocarditis:
Vasculitis that has not resolved within 6 months prior to study drug.

(7) Clinically significant gastrointestinal disorders including:
Gastrointestinal perforation <6 months prior to study drug administration. Subjects must have documented evidence (e.g., upper endoscopy, colonoscopy) of completely healed area of prior perforation;
Gastrointestinal bleeding <2 months prior to study drug administration. Subjects must have documented evidence (e.g., upper endoscopy, colonoscopy) of completely healed area of prior bleeding;
Pancreatitis <6 months prior to the initiation of study drug. Subjects must have a CT scan negative for evidence of remaining disease or normal pancreatic enzyme levels for >4 weeks prior to the initiation of study drug;
Diverticulitis flare <2 months prior to study drug administration. Subjects must have a CT scan negative for evidence of remaining disease prior to the initiation of study drug;
History of Crohn's disease or ulcerative colitis.

(8) Inflammatory process that has not resolved for ≥4 weeks from the date of first study dose. Subjects with chronic low-grade inflammatory processes such as radiation-induced pneumonitis are excluded regardless of duration.

(9) Clinically significant pulmonary compromise (e.g., requirement for supplemental oxygen on a continuous basis).

(10) Active viral, bacterial, or systemic fungal infection requiring parenteral treatment within 7 days prior to the initiation of study drug. Systemic antiviral, antifungal, or antibacterial therapy must be completed >1 week prior to the initiation of study drug. Antimicrobial prophylaxis (e.g., for Pneumocystis carinii infection) may continue the antimicrobial for that purpose.

(11) Vaccination with any live virus vaccine within 4 weeks prior to the initiation of study drug administration. Inactivated annual influenza vaccination is allowed.

(12) Subjects who are known to be human immunodeficiency virus (HIV) positive or hepatitis B or C positive and have uncontrolled disease. Subjects treated for hepatitis C must have viral titers of 0 for ≥2 years to be eligible. Subjects with hepatitis B having undetectable or ≤500 IU/mL hepatitis B viral DNA are eligible for the investigational product. Subjects treated for HIV must have CD4+ T cell (CD4+) counts ≥350 cells/μL and HIV viral load less than 400 copies/mL to be eligible.

(13) Second primary invasive malignancy not in remission for ≥1 year. Exceptions include non-melanoma locally advanced skin cancer, cervical carcinoma in situ, localized prostate cancer (Gleason score ≤7), resected melanoma in situ, or any malignancy considered to be indolent and never required systemic therapy, with the exception of indolent lymphomas.

(14) Any serious underlying medical or psychiatric condition that would preclude understanding and rendering of informed consent or impair the ability of the subject to receive or tolerate the planned treatment.

(15) Known hypersensitivity to Compound 1 or any excipient (trehalose, histidine, arginine, or polysorbate-80) contained in the drug or diluent formulation.

(16) Any medical or non-medical issue that would contraindicate the subject's participation in the study or confound the results of the study.

(17) Pregnant, likely to become pregnant, or lactating women (where pregnancy is defined as the state of a female after conception and until the termination of gestation).

Efficacy Assessments: Tumor assessments will be obtained at Screening using CT and/or MRI scans plus any necessary tumor markers or other exams at time intervals as specified in the Schedule of Events. Disease status will be evaluated every 8 weeks (56 days) using CT and/or MRI plus tumor markers and other measures as appropriate for the sites of disease, accompanied by physical examination. For both Phase 1 and Phase 2, subjects' tumor status will be evaluated every 8 weeks (56 days) per both iRECIST and RECIST v1.1.

### Study Treatment

Study Drug: Compound 1 for this study is an anti-Vβ6/IL-2 fusion protein and will all be open-label administrations. The formulation is a solution containing 10 mg/mL of Compound 1. Unit does strength is 10 mg/mL. Route of administration is: intravenous (IV) infusion over 30 f 5 minutes or 60 f 5 minutes (Sponsor may increase the infusion duration to 45±5 minutes to 90±5 minutes if the shorter infusion is found to be less tolerable)

### Treatment Administered

Phase 1 Compound 1 Administration: For Phase 1, the initial dose of Compound 1 will be infused over 60 minutes (±5 minutes), with all subsequent infusions administered over 30 minutes (±5 minutes) if deemed safe. Based on the safety and PK review of the 30-minute (±5 minutes) infusion, the Sponsor may increase the infusion duration to 45 to 90 minutes (±5 minutes) if the 30-minute (±5 minutes) infusion is found to be less tolerable. Compound 1 is administered Q2W and a cycle is defined as 4 weeks (28 days) within which Compound 1 is administered on Day 1 and Day 15.

Phase 2 Compound 1 Administration: The Compound 1 dose (the RP2D) and dosing regimen used in Phase 2, dose expansion, will be based on the safety, tolerability, and PK evaluation in Phase 1, dose escalation.

### Selection and Timing of Dose for Each Subject

### (A) Phase 1 Dose Escalation Selection and Timing of Dose.

Dosing in Phase 1 will be staggered. All dose escalation decisions will be made by the SRC. Dose escalation decisions will be supported by clinical safety and all available PK and pharmacodynamic data during the DLT Evaluation Period. Intermediate dose levels and alternative dosing schedules may be explored selectively during the Phase 1, Dose Escalation phase, of the study, based on review of the cumulative safety data and upon agreement with the SRC. Any decision to implement an intermediate dose level or alternative dosing schedule will be documented and submitted to participating IRBs/IECs and regulatory authorities, as appropriate, per local requirements.

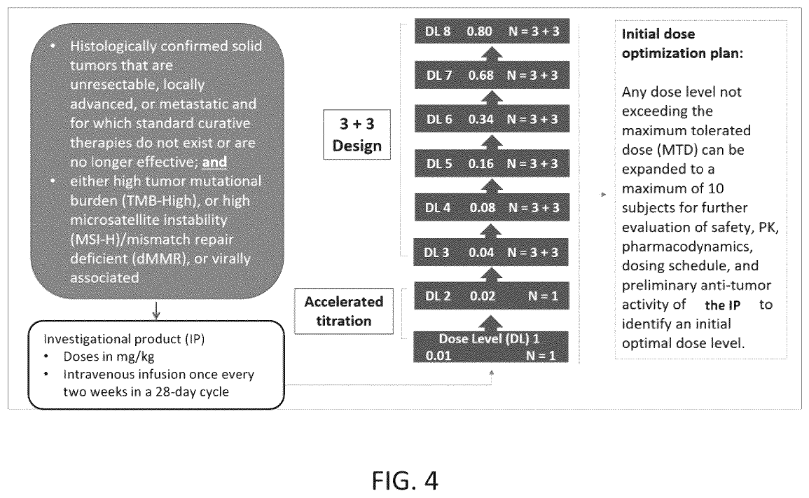

The trial design of Phase 1 (a hybrid design with an initial dose optimization plan) is shown in FIG. 4. In Phase 1, subjects who experience infusion delays >6 consecutive days during the DLT Evaluation Period due to non-dose-limiting drug-related toxicity or other non-drug related events (e.g., unavoidable travel delays, holidays), may remain on treatment, but will not be considered evaluable for DLTs, and will be replaced in that dose level. The decision to dose-escalate to the next dose level will be based on the SRC analysis of the aggregate safety, and available PK and pharmacodynamic data on all subjects participating in the enrolled dose level through the DLT Evaluation period.

Subject enrollment and dosing will be based on the following hybrid of an accelerated-titration and standard 3+3 design: (1) If 0 subjects experience a DLT at any completed dose level, then the study may proceed to the next dose level. (2) Dose levels 1 and 2, which begin with 1 subject each, will be switched to a 3+3 design if any ≥Grade 2 toxicity that is at least possibly related to Compound 1 occurs in each dose level. (3) At and after Dose level 3, cohorts will begin with 3 subjects. If one DLT occurs at any given dose level, 3 additional subjects may be added to that dose level for a total of 6. If a second DLT occurs at this dose level, recruitment/treatment/escalation will be halted. If just 1 DLT is observed in 6 subjects, escalation may continue. (4) If more than 1 DLT occurs at any dose level, it will be considered dose-limiting and additional subjects will be tested at the immediately previous lower dose level to ensure that a total of 6 subjects have been treated safely at that dose. The MTD is defined as the highest dose level at which <33% of subjects experience a DLT during the DLT Evaluation Period. In the situation where there is no lower dose level (i.e., if limiting dose is confirmed in dose level 1), the SRC will make a recommendation regarding study continuation or termination. (5) For initial dose optimization, any dose escalation cohort not exceeding the MTD can be expanded to a maximum of 10 subjects for further evaluation of safety, PK, pharmacodynamics, and preliminary anti-tumor activity of the investigational product. The additional group of subjects added to a dose escalation cohort are termed backfill subjects. (6) Intermediate dose levels may be explored based on emerging data (safety, pharmacodynamic observations) and if explored, will be discussed and agreed by the SRC and such decisions will be documented in writing. (7) As an additional safety measure, the SRC can deny a planned dose escalation at any time and request to treat additional subjects at a certain dose level, even in absence of a DLT. This refers to the occurrence of any non-DLT-level AE that raises concerns regarding safety of study subjects and treatment continuation. This request may also suggest dosing additional subjects at a lower dose level.

Upon confirmation that the first subject has tolerated the study drug and there are no significant toxicities that preclude further dosing, additional subjects may be dosed in a staggered fashion. There will be an interval of at least 5 days between the initial dosing of the first two subjects in each cohort starting from Dose Level 3.

The next higher dose cohort will not start until the previous dose cohort has met the criteria for dose escalation and written or verbal (recorded in minutes) agreement from the SRC has been obtained.

Phase 1—Staggering: Subjects will be assigned to a dose level in the order of study entry. There should be an interval of at least 5 days between initiating treatment of the first and second subject in each new dose escalation cohort to allow for initial assessment of safety before another subject may be dosed. This 5-day staggering interval is anticipated to provide a time window adequate to evaluate any acute serious toxicities arising from activation of the immune system, which are generally expected to occur between the first hours to up to 3 days after drug exposure. Upon confirmation that the first subject has tolerated the study drug and there are no significant toxicities that preclude further dosing, the next subject may be dosed in a staggered fashion. In the event of a de-escalation to a dose already deemed safe, a delay in administration is not required. The Investigator will review each case before allowing the next subject to be dosed.

Re-treatment: Subjects who remain clinically stable and do not experience DLTs or unacceptable toxicity during Cycle 1 (28 days) may continue treatment with Compound 1 until confirmed disease progression, initiation of alternative cancer therapy, unacceptable toxicity, withdrawal of consent, or other reasons to discontinue treatment occur. Subjects who are eligible for re-treatment may receive Compound 1 at a dose level that they have previously tolerated or at a higher dose level that has been shown to be safe based on subsequent dose level cohorts.

### (B) Phase 2 Dose Expansion Selection and Timing of Dose

The RP2D to be used in Phase 2 of the study will be determined based on data from Phase 1 of the study. Eligible subjects will be enrolled and assigned to a cohort in Phase 2 based on the subject's diagnosis.

### (C) Dose Adjustment.

Dose adjustments after Cycle 1 may be allowed at the discretion of the Investigator after discussion with the Sponsor Subjects may be allowed to continue study drug at a reduced dose if this is judged to be in their best interest. Compound 1 dose reductions and interruptions should be considered as outlined in the General Guidelines for Dose Reduction and Interruption (table below). In addition: (1) Subjects who experience toxicities that could have been qualified as DLTs may, at the discretion of the Investigator and after discussion with the Sponsor, be allowed to continue study drug at the reduced dose according to the dose modification guidelines; (2) For subjects who require dose interruption due to Compound 1-related toxicity in Cycle 2 or beyond, the treatment may re-start once the toxicity has been resolved to Grade ≤1 or to baseline.

TABLE E10
General Guidelines for Investigational Product (Compound 1) Dose
Reduction and Interruption

| | | |
|---|---|---|
| Compound 1- | | Approximate |
| related Toxicity^{a} | During Therapy | Dose Adjustment |

Grade 1

| | | |
|---|---|---|
| All occurrences | Continue the investigational | Maintain dose level. |
| | product treatment. | |
| | If CRS, then interrupt the | |
| | investigational product | |
| | until Grade 0. | |

Grade 2^{b}

| | | |
|---|---|---|
| 1^{st} occurrence | Interrupt the investigational | Maintain dose level. |
| | product until resolved to | If CRS, consider |
| | Grade ≤1 or baseline. | dose reduction. |
| | If CRS, then interrupt the | |
| | investigational product | |
| | until Grade 0^{c}. | |
| 2^{nd} occurrence | Interrupt the investigational | Consider reduction |
| (same toxicity) | product until resolved to | by 1 dose level of |
| | Grade ≤1 or baseline. | starting dose if |
| | If CRS, then interrupt the | dose delayed. |
| | investigational product | If CRS, then |
| | until Grade 0^{c}. | consider further dose |
| | | reduction. |
| 3^{rd} occurrence | Interrupt the investigational | Reduce by 1 or 2 |
| (same toxicity) | product until resolved to | dose levels of starting |
| | Grade ≤1 or baseline^{c}. | dose, if appropriate. |
| | If CRS, then discontinue the | |
| | investigational product. | |
| 4^{th} occurrence | Interrupt the investigational | Discuss with |
| (same toxicity) | product until resolved to | the Sponsor. |
| | Grade ≤1 or baseline^{c}. | |

Grade 3^{b}

| | | |
|---|---|---|
| 1^{st} occurrence | Interrupt the investigational | Reduce by 1 dose |
| | product until resolved to | level of starting dose. |
| | Grade ≤1 or baselines. | |
| | If CRS. then discontinue the | |
| | investigational product. | |
| 2^{nd} occurrence | Interrupt the investigational | Reduce by 2 dose |
| (same toxicity) | product until resolved to | levels of starting |
| | Grade ≤1 or baseline^{c}. | dose |
| 3^{rd} occurrence | Discontinue the investigational | Not applicable |
| (same toxicity) | product treatment. | |

Grade 4^{d}: Discontinue the investigational product
Abbreviations: ASTCT = American Society for Transplantation and Cellular Therapy; CRS = cytokine release syndrome.
For grading, see Common Terminology Criteria for Adverse Events (CTCAE v. 5.0); For CRS grading, see ASTCT consensus criteria
^{a}Excluding alopecia, anemia, lymphocytopenia, and asymptomatic neutropenia. Initiate optimal medical management for nausea, vomiting, diarrhea, and/or fever before any Compound 1 interruption or dose reduction.
^{b}Applicable only to those Grade 2 toxicities judged by the subject and Investigator to be intolerable and to all Grade 3 toxicities.
^{c}Interruption of Compound 1 treatment for more than 21 days (due to Compound 1-related toxicities) will require a discussion with the Sponsor before treatment can be resumed.
^{d}Excluding laboratory abnormalities judged to be non-life threatening, in which case manage as Grade 3.

### (D) Infusion Reaction Treatment Guidelines:

TABLE E11
Infusion reaction treatment guidelines are provided in the following table:

| | | Premedication |
|---|---|---|
| | | at Subsequent |
| NCI CTCAE Grade | Treatment | Dosing |
| Grade 1: | Increase monitoring of vital signs | None |
| Mild reaction; | as medically indicated until the | |
| infusion interruption | subject is deemed medically | |
| not indicated; | stable in the opinion of the | |
| intervention not | Investigator. | |
| indicated | | |
| Grade 2: | Stop infusion and | Subject may be |
| Requires infusion | monitor symptoms. | premedicated |
| interruption but | Additional appropriate | 1.5 hours |
| responds promptly | medical therapy may include | (±30 minutes) |
| to symptomatic | but is not limited to: | prior to infusion |
| treatment (e.g., | IV fluids | of Compound 1 |
| antihistamines, | Antihistamines | with: |
| NSAIDS, narcotics, | NSAIDS | Diphenhydramine |
| IV fluids); | Acetaminophen | 50 mg PO (or |
| prophylactic | Narcotics | equivalent dose |
| medications | Increase monitoring of vital | of antihistamine). |
| indicated | signs as medically indicated | Acetaminophen |
| for ≤24 hours | until the subject is deemed | 500 to 1000 mg |
| | medically stable in the | PO (or equivalent |
| | opinion of the Investigator. | dose of |
| | If symptoms resolve | antipyretic). |
| | within 1 hour of stopping | |
| | drug infusion, the infusion | |
| | may be restarted at 50% | |
| | of the original infusion rate | |
| | (e.g., from 100 mL/hr | |
| | to 50 mL/hr). | |
| | Otherwise, dosing will be | |
| | held until symptoms resolve | |
| | and the subject should be | |
| | premedicated for the | |
| | next scheduled dose. | |
| | Subjects who develop Grade 2 | |
| | toxicity despite adequate | |
| | premedication should be | |
| | permanently discontinued from | |
| | further trial treatment | |
| | administration. | |
| Grades 3 or 4 | Stop infusion. | No subsequent |
| Grade 3: | Additional appropriate | dosing |
| Prolonged (i.e., not | medical therapy | |
| rapidly | may include but is not limited to: | |
| responsive to | IV fluids | |
| symptomatic | Antihistamines | |
| medication and/or | NSAIDS | |
| brief interruption of | Acetaminophen | |
| infusion); recurrence | Narcotics | |
| of symptoms | Oxygen | |
| following initial | Vasopressors | |
| improvement; | Corticosteroids | |
| hospitalization | Epinephrine | |
| indicated for other | Increase monitoring of vital | |
| clinical sequelae | signs as medically indicated | |
| renal impairment, | until the (e.g.subject is deemed | |
| pulmonary | medically stable in | |
| infiltrates) | the opinion of the | |
| Grade 4: | Investigator. | |
| Life-threatening; | Hospitalization may be indicated. | |
| pressor, or | Subject is permanently | |
| ventilatory | discontinued from further trial | |
| support indicated | treatment administration. | |

Abbreviations: IV = intravenous; NSAID = Nnn-steroidal anti-inflammatory drugs; PO = taken by mouth.

### (E) Treatment After Radiologic Progression of Disease.

If a subject has initial radiologic progression while waiting the repeat imaging for confirmation per iRECIST, or has confirmed progressive disease (PD) (i.e., 2 scans at least 28 days apart demonstrating PD), the subject may continue Compound 1 treatment if all the following criteria are met and, if in the opinion of the Investigator, the subject will continue to receive benefit from treatment: (1) Absence of signs and symptoms indicating clinically significant progression of disease; (2) No decline in ECOG performance status; (3) Absence of symptomatic rapid disease progression requiring urgent medical intervention (e.g., symptomatic pleural effusion, spinal cord compression). (4) Documentation of the re-consent of the subject using a written informed consent document that details available therapies with potential clinical benefit that the subject may be foregoing in order to continue receiving Compound 1.

### (F) Intervention After the End of the Study.

During this study, subjects will continue treatment with the investigational product, Compound 1, until disease progression or unacceptable toxicity. However, no intervention will be provided to study subjects after the end of the study.

### Investigational Product Storage and Accountability.

The investigational product is a sterile liquid for intravenous infusion. It is formulated at a target concentration of 10 mg/mL of Compound 1 in 20 mM L-histidine/L-histidine monohydrochloride buffer, 8% (w/v) sucrose, 0.02% (w/v) polysorbate 80 at pH 6.0. Each vial is filled with 5 mL for a total of approximately 50 mg/vial. The formulated investigational product is stored at 5±3° C. before dilution for administration.

On the day of administration, the investigational product is diluted to a concentration of 0.2 to 1.5 mg/ml (depending on the dose) in 0.9% saline solution. The investigational product will then be infused immediately using syringe or IV bag systems. The investigational product can be stored refrigerated or at room temperature up to 24 hours in syringe or IV bag.

### Safety Assessments

### Dose Limiting Toxicities

The severity of AEs will be graded according to the NCI-CTCAE v 5.0 for non-CRS toxicities/infusion reaction while the CRS toxicity will be graded per ASTCT consensus guideline. Subjects who do not experience a DLT but are not evaluable for safety for the full DLT Evaluation Period will be replaced in the same dose level cohort. The investigational product dosing should be withheld for AEs potentially meeting criteria for a DLT pending resolution of the event and the investigational product attribution assessment from discussion between the Sponsor and the SRC. If this event type occurs outside of the DLT window or during Phase 2, dosing should be withheld under the same criteria. Safety data from dose escalation and expansion cohorts will be reviewed by the Principal Investigator, and all dose escalation decisions will be made by SRC.

A DLT is defined as any of the following AEs occurring during the DLT Evaluation Period of Cycle 1, graded using the NCI-CTCAE, Version 5.0, which are possibly, probably, or definitely related to the investigational drug and will include: (1) Any death not clearly due to the underlying disease or extraneous causes; (2) Any toxicity that results in a >14-day treatment delay; (3) Non-hematologic toxicity: (a) Any toxicity ≥Grade 3 with the exceptions as noted below: (b) Hy's law cases: (i) Aspartate aminotransferase (AST) and/or alanine aminotransferase (ALT)>3× upper limit of normal (ULN); (ii) Concurrent elevation of total bilirubin >2×ULN: (iii) No alternative etiology can be identified; (c) Any AST or ALT elevation >8×ULN regardless of duration and AST or ALT elevation 5 to 8×ULN that persists for greater than 2 weeks—all regardless of the presence or absence of liver metastasis; (4) Hematologic toxicity: (a) Grade 4 lymphopenia, neutropenia, or thrombocytopenia lasting >7 days (transient peripheral lymphopenia is expected per the mechanism of action of Compound 1); (b) Grade ≥3 neutropenia associated with complications secondary to infection and neutropenic fever lasting >48 hours; (c) Grade 3 thrombocytopenia with clinically significant bleeding.

DLT exceptions: (1) Grade 3 electrolyte abnormality lasting <72 hours, without associated clinical complications, and responding to therapy; (2) Grade 3 fever lasting <72 hours and not associated with hemodynamic instability: (3) Grade 3 nausea or vomiting resolving to Grade ≤1 within 72 hours with or without medical intervention; (4) Grade 3 amylase and/or lipase elevation not associated with either clinical or radiographic evidence of pancreatitis requiring hospitalization: (5) Grade 3 diarrhea, constipation or abdominal pain resolving to Grade ≤1 within 72 hours with or without medical therapy; (6) Grade 3 fatigue resolving to Grade 1 within <7 days; (7) ASTCT Grade 3 CRS/infusion reaction lasting ≤12 hours and responding to medical intervention. Grading is based on overall CRS/infusion reaction event, and not grade of individual signs or symptoms including fever, chills, nausea or vomiting, diarrhea, hypotension, hypertension, or tachycardia: (8) Grade 3 skin toxicity resolving to Grade ≤2 within 7 days of initiation of oral corticosteroids; (9) Grade 3 inflammatory reaction secondary to anti-tumor response such as rash and lymph node pain resolving to Grade ≤1 within 7 days.

### Pregnancy

Any pregnancy in a female subject (or partner of a male subject) in which the estimated date of conception is either before the last visit or within 30 days of last study drug, or any exposure to study drug through breastfeeding during study treatment or within 60 days of last study treatment, must be reported. If an adverse outcome of a pregnancy is suspected to be related to study drug exposure, this should be reported regardless of the length of time that has passed since the exposure to study drug. A congenital anomaly, death during perinatal period, an induced abortion, or a spontaneous abortion are considered to be an SAE and should be reported in the same time frame and in the same format as all other SAEs. All pregnancies must be followed to outcome. The outcome of the pregnancy must be reported as soon as possible but no later than 1 business day from the date the Investigator becomes aware of the outcome. A subject who becomes pregnant should be immediately withdrawn from the study. However, a subject who becomes pregnant may remain in the study if the Investigator judges that the potential benefit to the subject outweighs any potential risk to the subject or the fetus and the subject gives informed consent for the further participation. If a pregnancy is reported, the Investigator should inform the Sponsor within 24 hours of learning of the pregnancy. Follow-up information regarding the course of the pregnancy, including perinatal and neonatal outcome and, where applicable, offspring information must be reported. Abnormal pregnancy outcomes (e.g., spontaneous abortion, fetal death, stillbirth, congenital anomalies, ectopic pregnancy) are considered SAE. Generally, follow-up will be no longer than 6 to 8 weeks following the estimated delivery date. Details of all pregnancies in female partners of male subjects will be collected after study drug administration and until 30 days following study drug administration. In order for the Sponsor or designee to collect any pregnancy surveillance information from the female partner of a male subject, the female partner must sign an ICF for disclosure of this information. The expected date of delivery or expected date of the end of the pregnancy, last menstruation, estimated conception date, pregnancy result and neonatal data, etc., should be included in the pregnancy surveillance information.

### Severity

The assessment of severity of an AE will be rated according to the CTCAE v5.0 criteria with high level definition summarized in the following table.

TABLE E12
Definitions of adverse event severity.

| | |
|---|---|
| Grade 1 (Mild) | Mild; asymptomatic or mild |
| | symptoms; clinical or diagnostic |
| | observations only; intervention |
| | not indicated. The AE does not |
| | interfere with routine activities. The |
| | subject may experience slight |
| | discomfort. |
| Grade 2 (Moderate) | Moderate; minimal, local or |
| | noninvasive intervention indicated; The |
| | AE interferes with routine |
| | activities. The subject may experience |
| | significant discomfort. |
| Grade 3 (Severe) | Severe or medically significant |
| | but not immediately life threatening; |
| | hospitalization or prolongation |
| | of hospitalization indicated. The |
| | subject is unable to perform |
| | routine activities. The subject may |
| | experience intolerable discomfort or pain. |
| Grade 4 (Life | Life-threatening consequences; |
| Threatening) | urgent intervention indicated. |
| Grade 5 (Fatal) | Death related to AE |

Abbreviations: AE = adverse event; CTCAE = Common Terminology Criteria for Adverse Events. Based on the CTCAE v 5.0.

The term “severe” is use to describe the intensity of an AE; the event itself could be of relatively minor clinical significance (e.g., ‘severe’ headache). This is not the same as “serious.” Seriousness of AEs is based on the outcome of an AE and usually associated with events that pose a threat to a subject's life or functioning.

Vital Signs: Vital signs include heart rate, respiratory rate, temperature, and blood pressure (systolic and diastolic).

12-Lead Electrocardiograms: A 12-lead ECG will be performed with conventional lead placement. The subject will remain supine for at least 3 minutes prior to the ECG. The ECG results will be read by the Investigator or designee and results documented.

### ECOG Performance Status

ECOG performance status will be assessed and documented as the following: 0=Fully active, able to carry out all pre-disease activities without restriction. 1=Restricted in physically strenuous activity but ambulatory and able to carry out work of a light or sedentary nature (e.g., light housework, office work). 2=Ambulatory and capable of self-care but unable to carry out any work activities; up and about more than 50% of waking hours. 3=Capable of only limited self-care, confined to bed or chair more than 50% of waking hours. 4=Completely disabled, cannot carry on self-care, totally confined to bed or chair. 5=Dead

Pharmacokinetic and Immunogenicity Assessments: PK and ADA blood sampling should be obtained according to the Schedule of Assessment. Details are also described in the Laboratory Manual for the scheduling timepoints, handling, labelling, and shipping of the PK and ADA blood samples. Based on review of data, changes to the sampling schedule may occur. It is essential that the actual time and date of collection of each blood sample be recorded in the subject's eCRF. The volume of blood to be collected per sample should be approximately 5 mL. Serum concentrations of Compound 1 will be measured using a validated immunoassay. One serum sample will be collected at each time point indicated for Compound 1 PK evaluation in the Laboratory Manual. The following Compound 1 PK parameters will be evaluated after single- and repeat-dose: Phase 1 AUC₀₋ₜ, AUC_{0-inf}, Cₘₐₓ, Tₘₐₓ, V_{SS}, t_{1/2}, and repeat-dose Cₘₐₓ and C_{trough}; Phase 2: Compound 1 population PK parameters in expansion cohorts at the RP2D (e.g., CL, Vd). Other PK parameters may also be reported, as applicable. The generation and characterization of ADAs directed against Compound 1 will be assayed using a validated, bespoke bridging immunoassay with appropriate confirmation assays and validated cut-points. Assessment of the binding specificity of any detected ADAs may be further assessed using other bespoke assays (e.g., neutralizing antibody (Nab) assays).

Biomarker Studies: In nonclinical animal studies, the mSTAR surrogate molecule mediates its anti-tumor activity through selectively activating and expanding Vβ6NP10 T cells in blood and in the tumor. Therefore, the Sponsor will measure the following pharmacodynamic biomarkers to assess activity in trial subjects: (1) Quantitation of Vβ6/Vβ10 T cell subset counts by flow cytometry of PBMCs, qualitative and semi-quantitative assessment of Vβ6/Vβ10 T cells in tumor tissue by IHC/IF, and quantitative assessment of Vβ6/Vβ10 TCR gene expression in tumor tissue at baseline, and during and after completion of IV Compound 1 infusions. (2) Investigation of activation and phenotypic states of T cells by measuring cytokines and soluble sCD25 or using gene expression assays in PBLs and tumor tissue samples at baseline, and during and after completion of IV Compound 1 infusions. (3) Changes in TCR repertoire usage using TCR-specific RNA sequencing (TCRseq) and measurement of phenotypic markers on isolated TILs in tumor tissue samples at baseline, and during and after completion of IV Compound 1 infusions. (4) Whether these biomarkers correlate with other trial endpoints (safety, tumor response, or resistance).

These pharmacodynamic biomarker studies together with PK and safety data and anti-tumor activity will contribute to the improvement of the current trial design and to future studies investigating the safety and efficacy of Compound 1.

For Phase 1 dose escalation, a baseline tumor sample and tumor biopsy on study are required only for selected subjects but are mandatory for all Phase 2 dose expansion subjects.

### Discontinuation Criteria

### Discontinuation of Study or Study Drug

An Investigator may discontinue a subject from the study drug for these primary reasons: (1) A protocol violation occurs; (2) AE(s)/SAE(s); (3) Pregnancy; (4) Occurrence of a drug-related DLT; (5) Drug interruption for ≥21 days; (6) Lost to follow-up: (7) The Sponsor or Investigator terminates the study; (8) Withdrawal of consent by subject or proxy; (9) Other.

Subjects who are confirmed to have disease progression during the study but otherwise are achieving clinically meaningful benefit may choose to continue the treatment at the discretion of the Investigator, if clinically stable defined as: (1) Absence of symptoms and signs indicating clinically significant progression of disease: (2) No decline in Eastern Cooperative Oncology Group (ECOG) performance status; (3) Absence of symptomatic rapid disease progression requiring urgent medical intervention (e.g., symptomatic pleural effusion, spinal cord compression).

### Study Endpoints.

### (A) Phase 1 Dose Escalation.

The Phase 1 primary endpoints are as follows: (1) DLTs: (2) Safety (including but not limited to): TEAEs, SAEs, deaths, clinical laboratory abnormalities per NCI-CTCAE, Version 5.0, and CRS per ASTCT criteria.

The Phase 1 secondary endpoints are as follows: (1) ORR according to iRECIST; (2) DOR defined as time from the date of first documented PR/CR until the first documentation of confirmed disease progression per both iRECIST and RECIST v1.1; (3) DCR defined as the proportion of subjects achieving CR+PR+SD per both iRECIST and RECIST v1.1; (4) Compound 1 PK parameters following single- and repeated IV infusions as data permit (e.g., AUCs, Cₘₐₓ, Tₘₐₓ, CL, Vss, single-dose t_{1/2}, and repeat-dose Cₘₐₓ and C_{trough}); (5) ADA incidence and titers after single- and repeat-IV dosing of Compound 1.

The Phase 1 exploratory endpoints are as follows: (1) Cell counts and frequency of Vβ6/Vβ10 T cell subsets in PBMCs by flow cytometry; (2) Expression levels of Vβ6/Vβ10 in T cells and other germline Vβ TCR variants in PBMCs and TILs in tumor tissue biopsy samples by gene expression analysis (e.g., using Nanostring assays); (3) Levels of serum cytokine sand other soluble factors (e.g., sCD25) by immunoassay following single- and repeat-dosing of Compound 1; (4) PFS defined as the time from the initial infusion of study drug until documented disease progression or death from any cause per both iRECIST and RECIST v1.1; (5) OS: (6) ORR, DOR, DCR, PFS per iRECIST and RECIST, and OS in subgroups of TMB-H, MSI-H/dMMR and virally associated cancers, with each subgroup of subjects combined across all dose cohorts. Subgroup analysis will be performed at the Sponsor's discretion.

### (B) Phase 2: Dose Expansion.

The Phase 2 primary endpoints are as follows: (1) ORR defined as the proportion of subjects who have CR+PR per iRECIST.

The Phase 2 secondary endpoints are as follows: (1) ORR per RECIST v1.1; (2) DOR per both iRECIST and RECIST v1.1: (3) DCR per both iRECIST and RECIST v1.1; (4) PFS defined as the time from the initial infusion of study drug until documented disease progression or death from any cause per both iRECIST and RECIST v1.1; (5) OS; (6) Compound 1 population PK parameters in expansion cohorts at the RP2D (e.g., Cₘ., AUC, CL, Vd); (7) Safety (including but not limited to): TEAEs. SAEs, deaths, clinical laboratory abnormalities per NCI CTCAE, Version 5.0, and CRS per ASTCT criteria: (8) ADA incidence and titers after single- and repeat-IV dosing of Compound 1.

The Phase 2 exploratory endpoints are as follows: (1) Cell counts and frequency of V6/V310 T cell subsets by flow cytometry; (2) Qualitative and semi-quantitative measurement of Vβ6/Vβ10 T cells in tumor tissue by Multiplex IHC/IF; (3) Changes in gene expression profile of T cells isolated from PBMCs, and TILs from tumor tissue: (4) TCR sequencing (TCRseq) of RNA extracted from TILs of the tumor biopsy tissue samples: (5) TIL phenotyping by flow cytometry (will be undertaken at selected sites with experience); (6) Circulating ctDNA in the peripheral blood; (7) EORTC QLQ-C30 and EQ-5D-5L questionnaires.

### Statistical Methods:

Efficacy: ORR. DOR. DCR, and PFS will be determined per iRECIST. Sensitivity analyses will also be performed using RECIST v1.1 for the following variables. (1) ORR will be calculated as the proportion of subjects who have CR+PR. Exact two-sided 95% CIs will be constructed around the ORRs. (2) DOR defined as time from the date of first documented PR/CR until the first documentation of confirmed disease progression or death whichever occurs first. (3) DCR defined as the proportion of subjects who remain free of disease progression (CR+PR+SD). Two sided exact 95% CIs will be constructed around the DCR. (4) PFS will be calculated as the time from the initial infusion of study drug until documented disease progression or death from any cause. Subjects with no PFS event (disease progression or death from any cause) will be censored at the date of their last tumor assessment. Any subject who dies after 2 or more missed assessments will also be censored at the date of their last tumor assessment. Kaplan-Meier estimates will be provided for the median PFS. (5) OS will be calculated as the time from the initial infusion of study drug to death from any cause. Subjects who do not die will be censored at the date that the subject was last known to be alive. Kaplan-Meier estimates will be provided for the median OS.

### Example 3: Additional Experiments

FIG. 4 shows Phase 1 Trial Design.

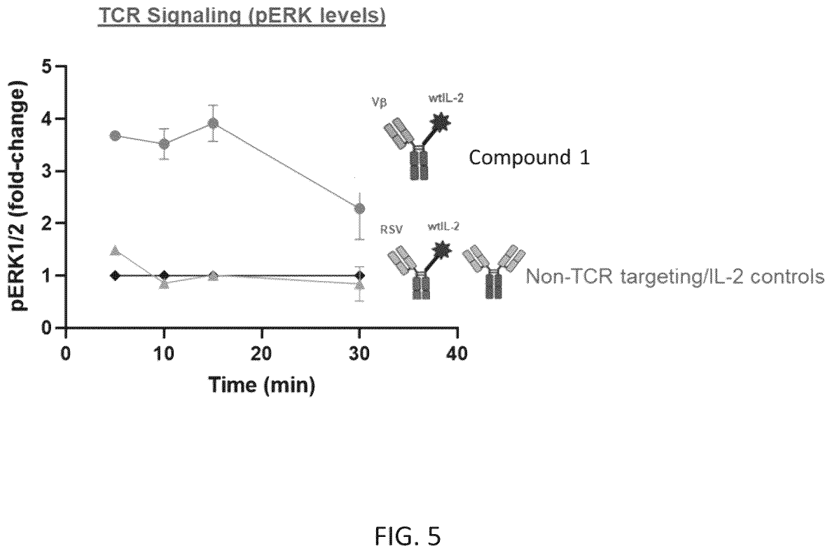

FIG. 5 shows that a multifunctional molecule containing an anti-TCRβVβ6 binding domain and an IL-2 domain (Compound 1) increases TCR signaling as measured by pERK level compared to a multifunctional molecule containing an non-TCR binding domain and IL-2 control and a multifunctional molecule containing two non-TCR binding domains control.

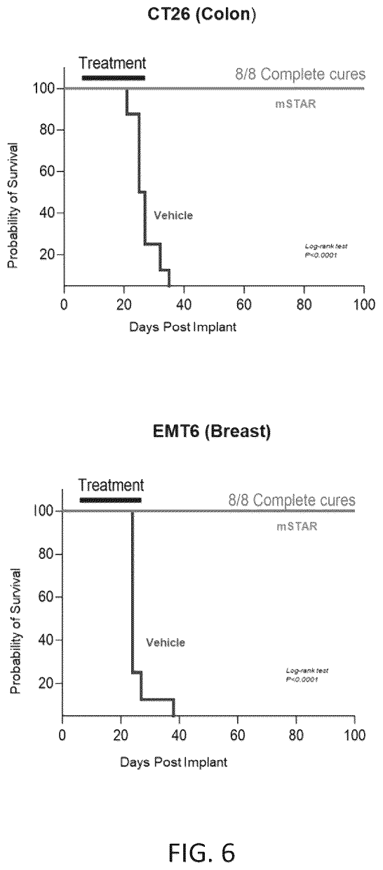

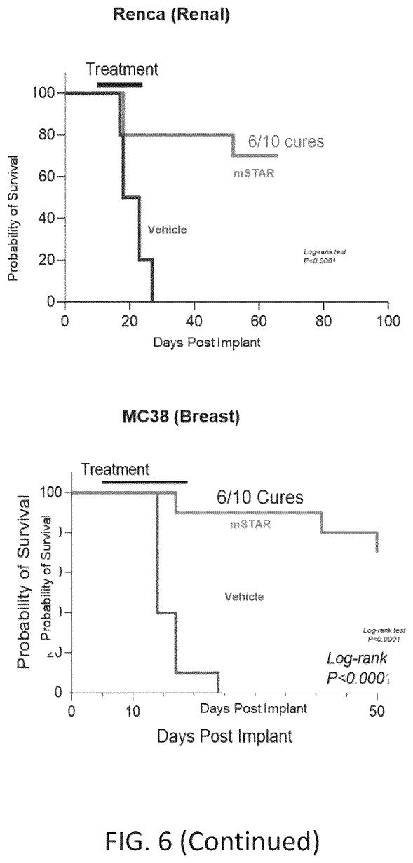

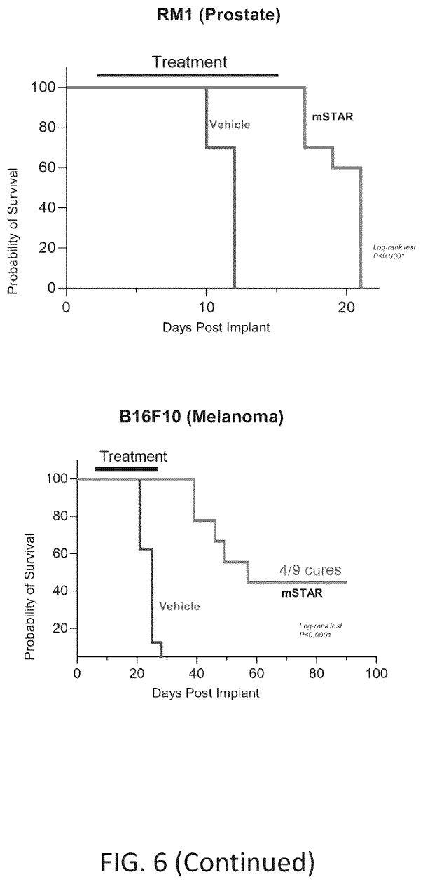

FIG. 6 shows potent single-agent activity of a murine surrogate bispecific antibody (BsAb) of Compound 1 (mSTAR) with durable response in various tumor models including PD-1 refractory models.

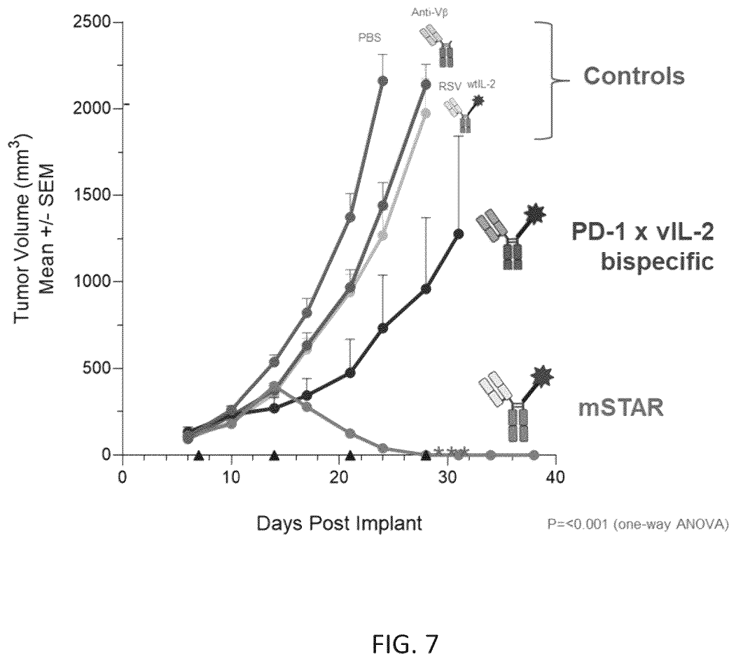

FIG. 7 shows that mSTAR leads to potent tumor regressions in EMT6 model.

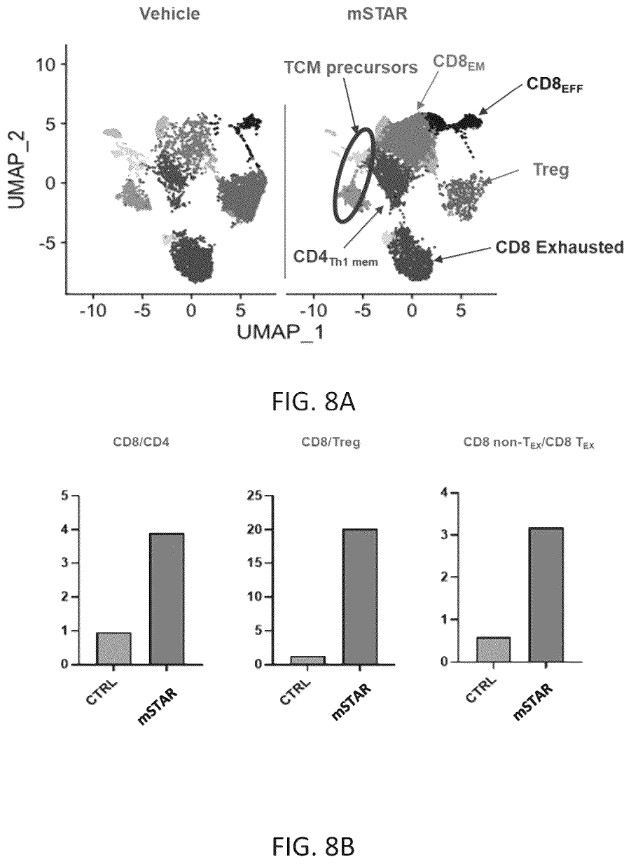

FIGS. 8A-8B show that mSTAR remodels tumor infiltrating lymphocytes (TILs), e.g., expansion of Vβ CD8+/CD4+ T effector memory (TEM) cells and Central memory T (TCM) cells. FIG. 8A shows scRNAseq analysis of EMT6 TIL. FIG. 8B shows scRNAseq analysis of TIL subtypes.

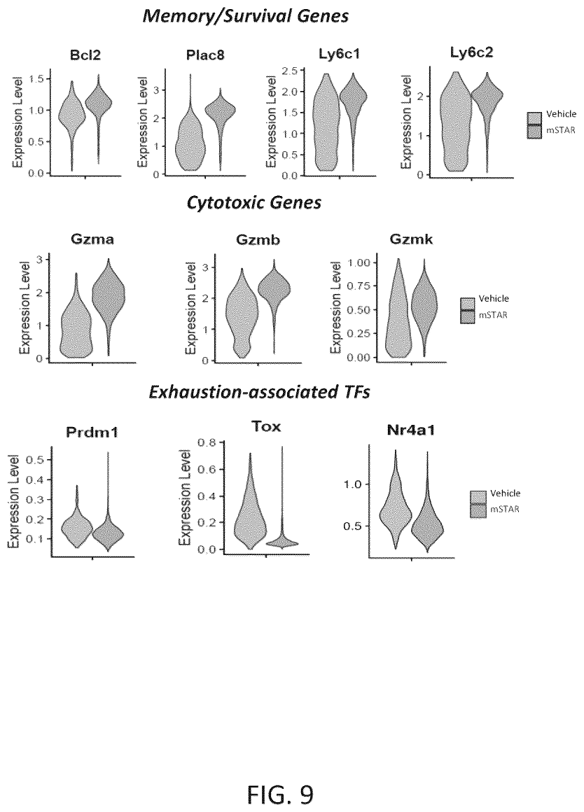

FIG. 9 shows that mSTAR induces a novel TEM phenotype.

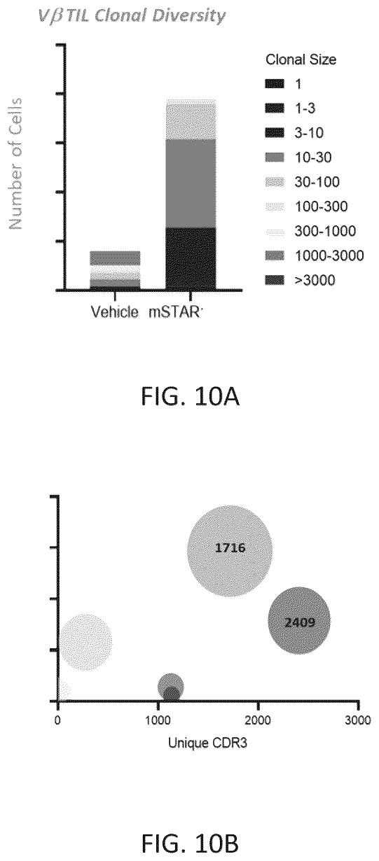

FIGS. 10A-10B show that mSTAR induces an increase in TCR diversity in TILs. FIG. 10A shows that mSTAR increases Vβ TIL Clonal Diversity. FIG. 10B shows large increase in unique CDR3 transcripts in TILs treated with mSTAR.

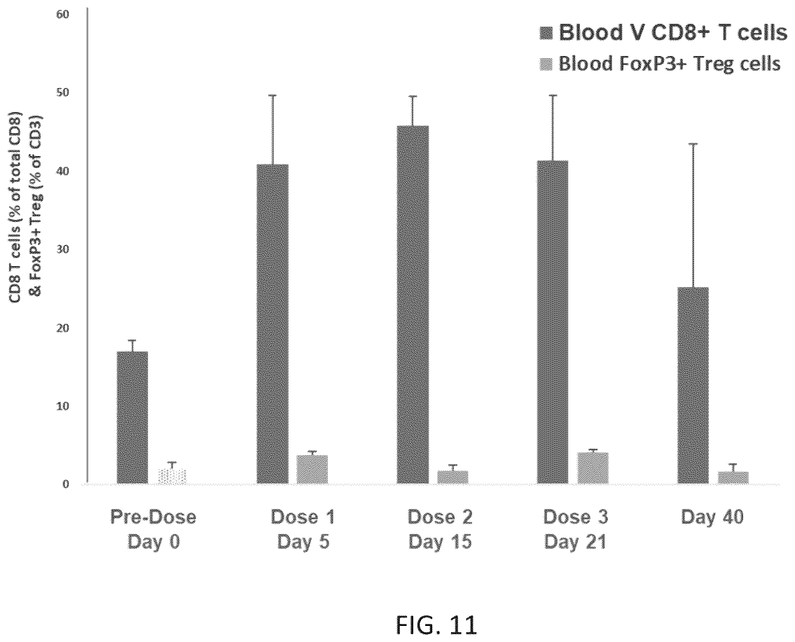

FIG. 11 shows that Compound 1 induced expansion of Vβ6 CD8+ T cells in blood of monkeys with minimal Treg.

FIG. 12 shows that Compound 1 induces ex vivo expansion of patient TILs and killing of refractory autologous tumors as compared to pembrolizumab.

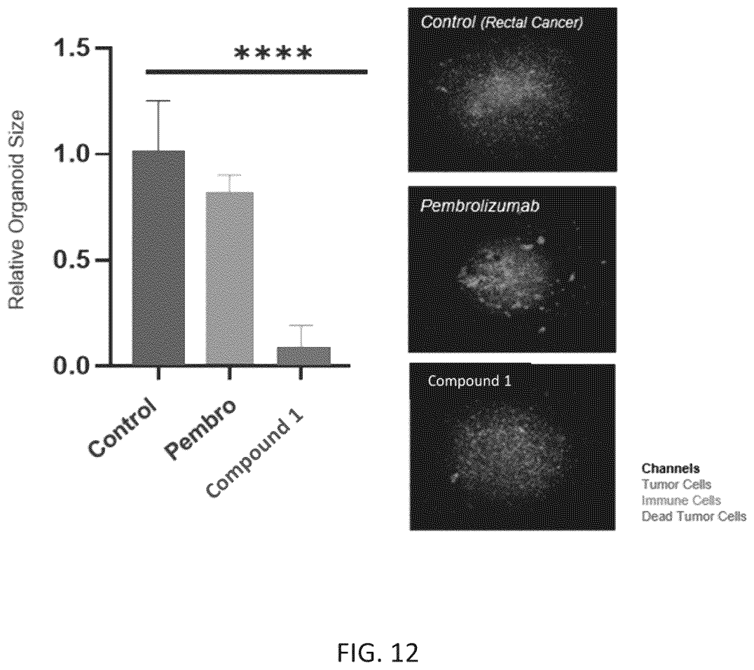

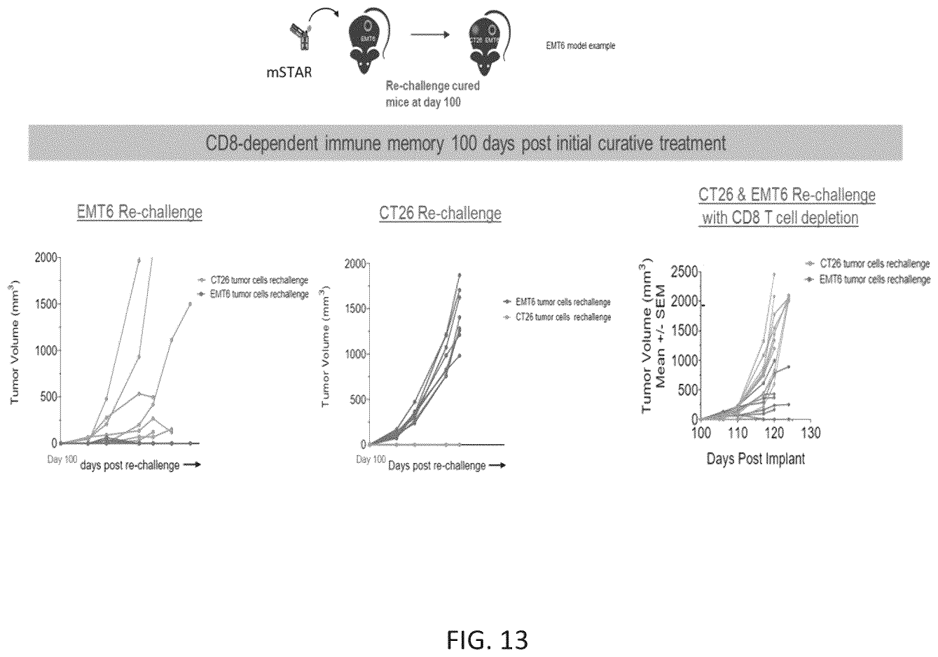

FIG. 13 shows mSTAR promotes “functional memory” & long-term protection as a result of Vβ CD8+ T cells.

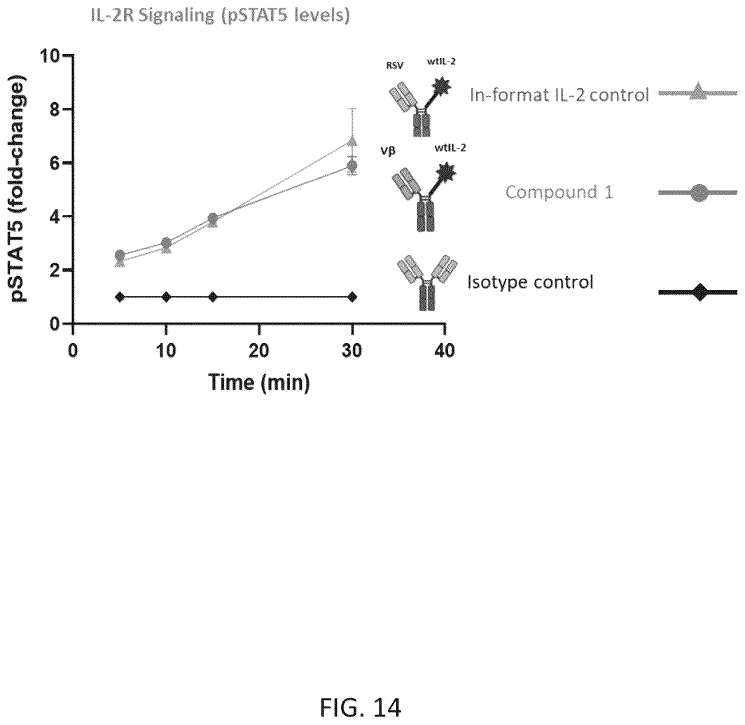

FIG. 14 shows that Compound 1 and a multifunctional molecule containing an non-TCR binding domain and IL-2 control increase IL-2R signaling as measured by pSTAT5 level compared to an isotype control.

### Example 4: A TCR P Chain-Directed Antibody-Fusion Molecule that Activates and Expands Subsets of T Cells and Promotes Antitumor Activity

### Methods

The following are methods used in this study examining a bifunctional molecule capable of inducing T cell activation and expansion and enhancing antitumor activity in CPI-refractory settings.

All sequences were synthesized by GeneArt (Thermo Fisher Scientific) and cloned into the mammalian expression vector pcDNA3.4 (Thermo Fisher Scientific). Each chain was added in a 1:1:1 weight/weight/weight ratio. Each molecule was transiently transfected in ExpiCHO-S cells (Thermo Fisher A29127) and harvested by centrifugation on Day 14 after transfection. Following harvest and filtration, the clarified cell culture supernatant was captured on a Protein A column and eluted using low pH buffer. Following the initial capture step, the Protein A eluate was diluted 10-fold in cation exchange (CEX) Buffer and applied to a Mono S™10/100 GL column (Cytiva 17516901) for further polishing. The material was buffer exchanged into formulation buffer and analyzed via analytical SEC (aSEC) and SDS-PAGE to determine purity.

All interactions between each construct binding domain(s) and the relevant receptor proteins were analyzed by Surface Plasmon Resonance (SPR) on a Biacore T200 instrument (Cytiva). Each construct at 2 ug/mL was immobilized on a Series S CM5 chip (Cytiva BR100530) via human or murine Fc antibody (Cytiva BR100839) to 80 RU. Human TCRβV16-5, 1L2Rα, IL2Rγ, and IL2R trimeric complex as well as cynomolgus TCRβVβ6-2, IL2Rα (Sino Biological 90265-C08H) and 1L2Rβγ were diluted to the appropriate starting concentration and then serially diluted two-fold in 1×HBS-EP+ buffer (Cytiva BR100669) for a total of 10 concentrations. Multi cycle kinetics was run where the chip is regenerated with 3 M Magnesium Chloride between each cycle followed by a new injection of construct. Dissociation in running buffer was performed at 30 uL/min. Sensorgrams were corrected by double reference subtraction using the reference flow cell not treated with construct and a blank cycle of buffer alone. BIAevaluation software (Cytiva) was used for data analysis and the data were fit using a 1:1 Langmuir binding model for calculation of the KD value (Koff/Kon). Due to faster off rates observed for some binding interactions, the data was fit using a steady state model where equilibrium binding response is analyzed.

Human PBMC (purchased from Stemcell Technologies) were isolated from blood from different healthy donors using density gradient separation. Murine whole blood was collected via heart puncture and placed in a 15 mL conical centrifuge tube. Blood was suspended in 5 mL cold ACK lysis buffer and pipetted gently up and down to mix, then incubated on ice for 5 min. Lysis buffer was then quenched by addition of 10 mL MACS buffer. Cells were centrifuged for 5 min at 300 g.

Murine tumor cell lines RENCA (CRL-2947), B16F10 (CRL-6475), CT26 (CRL-2638), RM1 (CRL-3310) and EMT6 (CRL-2755) were obtained from American Type Culture Collection. MC38 were obtained from NIH (ENH204-FP). Cell lines were tested for mycoplasma and other pathogens at Charles River Research Animal Diagnostics services and cultured according to their guidelines.

In general, cells were suspended in flow stain buffer composed of phosphate-buffered saline (PBS) and 0.5% bovine serum albumin (BSA), 2 mM EDTA followed with a choice of Cell Viability Dye, Zombie NIR (Biolegend. #423106). Zombie UV (Biolegend, #423108), or Fixable Viability Dye eFluor 780 (Thermofisher, #65-0865-14); diluted in PBS for 10 min on ice in the dark. Cells were washed with PBS and stained with Fc-block (Biolegend, #422302). Antibody(ies) for staining cell surface markers were added and incubated for 30 min on ice in the dark prior to being fixed and permeabilized (eBioscience, #00-5523-00) by following protocols provided by the manufacturer. Antibody(ies) for intracellular staining were added and incubated for 30 min on ice in the dark, followed by a cell washed and resuspended in flow stain buffer. CountBright Counting Beads (Thermofisher, #C36950) were added per manufacturer instructions for direct cell count quantitation. Details for cell staining antibody cocktails are described in each section.

For measuring Compound 1 binding to human CD4+ and CD8+ T cells, human PBMCs were suspended at 1×106cells/ml in flow stain buffer mixed with a serial dilution of Compound 1 or single arm controls, anti-TCRβVβ6/Vβ10 monovalent Fab and anti-RSV Fab x IL-2 or Isotype Control (all produced internally at Marengo Therapeutics), in PBS with a starting concentration of 100 nM for 30 min at 4° C. Cells were washed and stained with secondary antibody: Alexa Fluor 647 goat anti-human IgG (Jackson ImmunoResearch, #109-607-008) diluted 1:800 per 100 μl in flow stain buffer for 30 min on ice in the dark. Cells were washed for flow cytometry panel staining. Antibody cocktail for cell surface markers: Brilliant Violet (BV)-510 anti-human CD25 (clone M-A251, Biolegend, #356120), BV-650 anti-human CD8 (clone RPA-T8, Biolegend, #301042), BV-786 anti-human CD4 (clone L200, BD Bioscience, #563914). Peridinin-Chlorophyll (PerCP)-Cy5.5 anti-human CD3 (clone SP34-2, BD Bioscience, #552852). Phycoerythrin (PE) anti-human NKG2A (clone Z199, Beckman Coulter. #1M3291U), PE-Cyanine (Cy) 7 anti-human CD20 (clone 2H7, Biolegend, #302312), BV421 anti-human CD14 (clone M5E2, Biolegend, #301830). Antibodies for intracellular staining: Alexa Fluor 488 anti-human FoxP3 (clone 259D, BioLegend, #320212). Stained cells were analyzed using the Cytek Aurora (Cytek). CD4+ and CD8+ T cells were gated from single cells, live cells, CD20-CD14-CD3+ and FoxP3−.

To evaluate IL-2 bioreactivity for Compound 1, RSV Fab x IL-2, and rhIL-2, human PBMCs isolated from healthy donors were stimulated with serial dilution of Compound 1, RSV Fab x IL-2RSV and rhIL2 (Peprotech. Cat #200-02) starting at 100 nM for 5 mins. A warm fixation buffer was added to fix cells and stop the reaction. Cells were washed for flow cytometry panel staining. Antibody cocktail for cell surface markers: Brilliant Violet (BV)-510 anti-human CD3 (clone OKT3, Biolegend, #317332), Spark NIR 685 anti-human CD4 (clone SK3, Biolegend, #344658), Brilliant UltraViolet (BUV)-805 anti-human CD8 (clone RPA-T8, Thermofisher, #368-0088-42). Antibodies for intracellular staining: PE-Cy5 anti-human FoxP3 (clone 236A/E7, Thermofisher, #15-4777-42), PE anti-human pSTAT5 (clone A 17016B.Rec, Biolegend, #936904). Stained cells were analyzed using the Cytek Aurora (Cytek). CD3+ T cells were gated from single cells, live cells, and FoxP3−.

To analyze Compound 1 binding to Vβ6+ and Vβ6-Low T cells with high or low CD25 expression, total pan T cells were isolated from human PBMC using negative bead selection kit (Miltenyi, Cat #130-096-535) following the manufacturer's protocol. Purified T cells were washed and resuspended at 10×106cells/ml in PBS/BSA (0.5%), followed by staining/labeling with PE anti-Vβ6-5 (Biolegend, Cat #362410) at 5 μl per 100 μl of T cells. After 30 min of staining at room temperature, T cells were washed and resuspended at 10×106cells/ml in Xvivo15 media (Lonza, Cat #BE02-060F). Vβ6-5+ T cells were then sorted (2-way sort) using the purity mode on the SH800 Cell Sorter (Sony Biotechnology), with pan-T cells (non-sorted) serving as a Vβ6-5lowcontrol. Pan T cells and sorted Vβ6-5+ T cells were then expanded using the human T cell Activation/Expansion Kit, consisting of anti-CD3/CD28 beads, (Miltenyi, Cat #130-091-441) supplemented with recombinant human IL-2 (100 IU/ml, Peprotech, Cat #200-02) as described by following manufacturer's protocol. At day 14, T cells were washed, collected and/or cryopreserved for storage. An aliquot of expanded Vβ6-5+ T cells was stained for Vβ6-5 to confirm purity using PE anti-Vβ6-5 (Biolegend, Cat #362410), and BV421 anti-CD3 (Biolegend, Cat #317344). Aliquots of expanded pan T cells and Vβ6-5 T cells were allowed to rest for 3 days to allow down-regulation of CD25 for phenotype with CD25lowexpression. Serial dilutions of Compound 1 in PBS with a starting concentration of 250 nM was incubated for binding to cells with the following resulting phenotypes: a) Vβ6-5+CD25hib) Vβ6-5+CD25lowc) Vβ6-5lowCD25hid) Vβ6-5lowCD25low. Stained cells were analyzed using the Cytoflex (Beckman Coulter). CD3+ T cells were gated from single cells and live cells.

For characterizing TRBV germline expression and Compound 1 binding, RNA was extracted from P12-Ichiwaka (DSMZ, #ACC34) and HSB-2 (DSMZ, #ACC435) cell lines using the RNeasy Plus Kits (Qiagen, #74134). RNA was then counted for TRBV expression using the nCounter TCR Diversity Panel (Nanostring, #XT-CSO-HTCR). TRBV genes were counted and represented as a percentage of abundance. Compound 1 binding to T cell lines was performed the same as binding to human PBMC's.

For the flow cytometry analysis for Vβ6/Vβ10 T cell expansion from primary human PBMCs, purified human T cells were stimulated with 10 nM biotinylated-Compound 1 using EZ-Link™Sulfo-NHS-LC-Biotin, (Thermo Fisher, #A39257) followed by surface marker staining with biotinylated anti-TCRβV6/Vβ10 (Marengo. #BKM0259), PerCP-Cy5.5 anti-human-CD3 (clones UCHT1, Biolegend, #300430), BV-421 anti-human CD4 (clone OKT4, BioLegend, #317434), PerCP-Cy5.5 anti-human CD8 (clone SK1. BioLegend, #344710). Secondary antibody staining with Alexa Fluor 647 Streptavidin (Jackson ImmunoResearch, #016-600-084) were diluted 1:25 per 100p0 in flow stain buffer for 30 min on ice in the dark. Stained cells were analyzed using the Cytoflex (Beckman). CD8+T cells were gated from single cells, live cells, and CD3+.

To analyze Compound 1 competition with a T cell expansion assay, purified human T cells were preincubated with competing soluble IL2R and Vβ6-5 antigen or a mixture of both in a dose dependent manner starting at 500 nM for 30 minutes at room temperature. 10 nM of Compound 1 were added as stimulation to evaluate T cell activation (CD25+) and T cell expansion via relative cell counts for CD3+ T cells analyzed using the Cytoflex (Beckman). CD3+ T cells were gated from single cells, live cells.

Human PBMC or purified human T cells were stimulated with 10 nM of Compound 1 and cultured in X-Vivo 15 media (Lonza, #BE02-053Q) for 7 days. Anti-Vβ6/Vβ10 monovalent Fab fused to a disabled Fc, anti-RSV Fab x IL-2 constructs, and an isotype were included as single-arm controls. In addition, anti-Vβ6/Vβ10 monovalent Fab (disabled Fc) and a pan T cells anti-CD3 (disabled Fc) were immobilized on a plate for at 4° C. for 20 hrs at a concentration of 100 nM in PBS serving as a TCR-only-mediated T cell response (without IL-2). T cell central memory phenotype were quantified by flow cytometry staining. Cells were stained with biotinylated anti-Vβ6/Vβ10 monovalent Fab, followed by secondary antibody staining with Alexa Fluor 647 Streptavidin (Jackson ImmunoResearch. #016-600-084). Cells were washed and stained with antibody cocktail for cell surface markers: BV-786 anti-human CD4 (clone L200, BD Bioscience, #563914), PerCP-Cy5.5 anti-human CD8 (clone SKI, Biolegend, #344710). BV-510 anti-human CCR7 (clone G043H7, Biolegend. #353232). Fluorescein Isothiocyanate (FITC) anti-human CD45RA (clone 5H9, BD Biosciences, #556626), BV-605 anti-human CD95 (clone DX2, Biolegend, #305628), PE-Cy7 anti-human CD56 (clone QA17A16, BioLegend, #392412). Stained cells were analyzed using the Cytoflex (Beckman). Central memory T cells were gated from single cells, live cells, CD4+ or CD8+, TCRβVβ6/Vβ10+, CD95+ for CD45RA-CCR7+ expression. Separately, 24 hrs before intracellular staining (i.e. at Day 6), Brefeldin A solution (Biolegend, #420601) diluted 1:1000 were added to samples for the accumulation of cytokine in the Golgi Complex. Antibodies for intracellular staining: BV-421 anti-human Granzyme B (clone QAI8A28, BioLegend, #396414), BV-510 anti-human Interferon-gamma (clone 4S.B3, BioLegend, #502544). Stained cells were analyzed using the Cytek Aurora (Cytek). IFNγ and Granzyme B expression levels were gated from single cells, live cells, CD56-CD3+, and CD8+.

For the analysis measuring TCR/IL2R phosphor-proteomic signaling, purified CD8+ T cells (purchased from Stemcell Technologies) were stimulated with 10 nM of Compound 1 and cultured in X-Vivo 15 media (Lonza, #BE02-053Q) for 5, 10, 15, and 30 mins. Anti-Vβ6/Vβ10 monovalent Fab (10 nM), anti-RSV Fab x IL-2 (10 nM) constructs, and an isotype were included as single-arm controls. In addition, anti-Vβ6/Vβ10 monovalent Fab were immobilized on a plate for at 4° C. for 20 hrs at a concentration of 100 nM in PBS serving as a TCR-only-mediated T cell response. Alpha SureFire assay kits were followed with manufacturer's instructions for the quantification of Phospho/Total ERK1/2 (Thr202/Tyr204) (PerkinElmer, #MPSU-PTERK) and phospho/total STAT5 (Tyr694/695) (PerkinElmer, #MPSU-PTST5). Stimulation was stopped once reached desired timepoint by adding cell lysis buffer. Signaling phosphorylation is normalized to total protein and expressed as a fold-change from samples treated with the isotype control.

For murine immunohistochemistry, tissue tumor, lung and liver samples were fixed in 10% neutral buffered formalin for 24 hours and then transferred to 70% ethanol followed by processing into paraffin blocks and sectioning to approximate 4 μm. Primary antibodies for staining were anti-mouse CD8 (Cell signaling, #98941), anti-mouse Granzyme B (Cell signaling, #44153), followed by the using the Novolink Polymer Detection Systems (Leica Biosystems), as per manufacturer's protocol. Additional lung and liver section slides were also stained with hematoxylin and eosin (H&E stains). Stained section slides were scanned and analyzed on the Aperio scanner and software (Leica Biosystems).

For murine tissue dissociation and TIL isolation, tissue tumor, lung and liver samples were gently cut to about 1 mm³ pieces in gentleMACS C-tubes (Miltenyi Biotec, #130-093-237). Tissues were digested enzymatically containing 20 mM HEPES (Gibco, #15630080), 1 mg/mL Collagenase (Sigma, #C1639), 0.1 mg/mL DNase (Roche, #10104159001) for 30 mins in a 37° C. shaker with gentle agitation. The tissue pieces were gently dissociated with mechanical force using the GentleMACS Octo Dissociator (Miltenyi Biotec). Cells were then filtered through a 70 μm cell strainer and suspended cells were subsequently washed and centrifuged for 5 min at 300 g. After tissue digestion and dissociation, CD4+ and CD8+ TILs (for tumors only) were isolated using the mouse CD4/CD8 (TIL) MicroBeads (Miltenyi, #130-116-480) according to manufacturer's protocol.

For examining murine AH1 tumor antigen T cell staining in tumor infiltrating lymphocytes (TILs). TILs were isolated from CT26 tumor mice after 1 mg/kg mSTAR QW for 2 total treatment, AH1/gp70 tumor antigen (MBL International, #TM-M521-1) in CD8 T cells were stained and analyzed using the Cytoflex (Beckman). Vβ13+ T cells were gated from single cells, live cells, CD3+CD8+.

For murine single cell RNA sequencing and analysis, data output from Cell Ranger was imported in R and processed with Seurat (v4.1.1) for downstream analyses Cells with a threshold of genes counts between 400 and 3500, and mitochondrial genes representing less than 5% per sample were retained. Cell annotation was done using the ProjecTILs R package (v2.0.0, https://github.com/carmonalab/ProjecTILs) with default parameters, leveraging three independent reference atlases for TILs (https://spica.unil.ch/refs/TIL), viral-activated CD4 (https://spica.unil.ch/refs/viral-CD4-T) and viral-activated CD8 (https://spica.unil.ch/refs/viral-CD8-T). Cell annotation was further refined by manually grouping cells based on the gene expression of Cd8a and Cd4 as CD8+ and CD4+ T cells, respectively. Double negative cells were annotated as ‘other’ and the minimal fraction of double positive cells were removed. Sub-populations were identified based on the expression of markers for effector, memory, and exhausted T cells. Raw counts were imputed using the Rmagic package (v2.0.3, https://cran.r-project.org/web/packages/Rmagic/index.html) and results were used for violin plots and heatmap visualizations. Differential gene expression comparing groups was performed using Seurat's default Wilcox test applied to raw count data, excluding genes with a log FC<0.1 or expressed in less than 10% of the cells. Adjusted p-values reported by Seurat use Bonferroni correction at a significance threshold of <0.05 and were used to determine genes whose expression significantly changes across conditions. Cells were labelled positive for TRBV based on the gene expression of Trbv13-2 and Trvb13-3.

For murine pharmacokinetics analyses, naive Balb/c mice were injected IP with a single dose of mSTAR (His-tag construct produced at Marengo Therapeutics) at doses 0.5, 1 and 1.5 mg/kg. At the 0.5, 1, 2, 4, 6, 24, 48, 72 hr timepoints, 20p0 of blood were collected without anti-coagulants by tail prick. For the 168 hr timepoint, animals were euthanized by CO2 inhalation and the final collection was done via cardiac puncture. Blood was dispensed into a Wheaton conical-bottom 350 μl glass insert tube (VWR, #97045-060) which is placed into a 1.5 ml microcentrifuge tube and the blood is allowed to clot for 30 mins at room temperature. Serum samples were collected by centrifuging the tubes at 5000 rpm for 10 minutes for PK analysis. PK assessment of mSTAR was performed using MSD electrochemiluminescence (ECL) sandwich-based (MSD, XL55XA) immunoassays, following manufacturer's protocol. In brief, for mSTAR measurements, human IL-2 antibody (R&D Systems, #MAB202) used as the capture antibody, followed by a two-step biotin anti-6×His-tag antibody (“6×His” disclosed as SEQ ID NO: 4032) (Abcam, #Ab27025) and streptavidin SULFO-tag label (MSD, #R32AD-1) as the detection antibodies. Value of unknown concentrations were determined by PK-solver (Excel add-in) using the nonlinear regression of an 11-point standard curve to known concentrations of mSTAR, with the fourth order polynomial model.

For murine pharmacodynamics analyses, Balb/c mice were injected IP with a single dose of mSTAR at 1 mg/kg. At the 3, 24, 72, 120, and 168 hrs timepoints, animals were euthanized by CO2 inhalation for blood collection via cardiac puncture. Blood and tissue PD assessment for Vβ13+in balb/c mice was done by flow analysis. The list of antibodies used were as follows for cell surface staining: BV-510 anti-mouse CD8 (clone 53-6.7, Biolegend, #100752), BV-605 anti-mouse CD25 (clone PC61, Biolegend, #102036), BV-650 anti-mouse NKp46 (clone 29A 1.4, Biolegend, #137635), BV-711 anti-mouse CD19 (clone 6D5, Biolegend, #115555). BV-750 anti-mouse CD3 (clone 17A2, Biolegend, #100249), PerCP-Cy5.5 anti-mouse CD45 (clone 30-F11, Biolegend, #103132), APC-Cy7 anti-mouse CD49b (clone DX5, Biolegend, #108920), BV-480 anti-mouse CD4 (clone RM4-5, BD Biosciences, #565634). AlexaFluor 488 anti-C-tag (ThermoFisher, #7213252100). Cells were fixed and permeabilized for intracellular staining using FoxP3/Transcription factor staining buffer set (Thermofisher, #00-5523-00) and incubated with fluorochrome-conjugated antibodies for 30 min on ice, in the dark. The list of antibodies used were as follows for intracellular staining: BV-421 granzyme B (clone QA18A28, Biolegend, #396414), PE anti-TCRβVβ8.1, 8.2 (aka 13-2, 13-3) (clone MR5-2, Biolegend, #140104), AlexaFluor anti-mouse FoxP3 (clone 150D, Biolegend, #320012), eFluor 450 anti-Ki67 (clone SolA15, Invitrogen, #48-5698-82). Stained cells were analyzed using the Cytek Aurora. CD8+ T cells were gated from single cells, live cells, CD49b-NKP46-CD19-CD3+ and FoxP3−. Tregs were gated similarly with CD4+ and FoxP3+. Blood samples were also tested for blood biochemistry analysis using the VETSCAN VS2 Chemistry Analyzer (Zoetis). Other dosing regimens includes: mSTAR at 0.5 mg/kg, 1 mg/kg, 1.5 mg/kg QW for total of 2 treatments, and the more potent regimen with mSTAR at 1 mg/kg and RSV F(ab)2×(IL-2)2 at 1.5 mg/ml twice weekly for total of 3 treatments to evaluate IL-2 control effects.

For murine single cell RNA data analysis from published datasets, datasets were pulled from two different publications that comprise single cell RNAseq data from murine models that received treatments with anti-PD-1, IL-2 (Hashimoto et al., DOI: 10.1038/s41586-022-05257-0), and anti-PD-1/IL-2 (Deak et al., DOI: 10.1038/s41586-022-05192-0). The expression matrixes and metadata from these datasets were downloaded and converted to Seurat objects. QC consisted of filtering cells with a UMI count between 300 and 4000 and removing cells with less than 5% reads mapping to mitochondrial gDNA. The dataset was normalized (SCTransform), scaled and then subjected to clustering (unless annotation was already provided in the form of metadata) to ‘CD8+ effector T cell’-Hashimoto et al. or ‘Better effector T cells’-Deak et al., depending on the dataset. Differential gene expression analysis, comparing against vehicle control, was performed as described above for scRNAseq data from mSTAR treated mice and using the same thresholds. Then overlap was calculated using GeneOverlap R package with the 53 gene signature from dataset.

For non-human primate pharmacokinetics analyses, naïve cynomolgus monkeys (Macaca fascicularis, of Cambodian origin, of 2.3-4.9 years old with body weights in the range of 2-4 kg) were intravenous (IV) administered with a single dose of Compound 1 at doses 0.5, 1 and 1.5 mg/kg, 0.5 ml of whole blood was collected via venipuncture in Serum Separation Tubes (BD, #367989) before dosing, and at 15 min, and 2, 6, and 24 hrs, and day 3, day 5, day 8, day 15 timepoints. PK assessment of Compound 1 was performed using MSD electrochemiluminescence (ECL) sandwich-based (MSD, XL55XA) immunoassays, following manufacturer's protocol. For Compound 1 measurements, goat anti-Human Kappa (κ) light chain antibody (Bethyl Laboratories, #A80-115A) used as the capture antibody, followed by a two-step biotinylated anti-human IL-2 antibody (R&D Systems. #BAF202) and streptavidin SULFO-tag label (MSD, #R32AD-1) as the detection antibodies. Value of unknown concentrations were determined by PK-solver (Excel add-in) using the nonlinear regression of an I1-point standard curve to known concentrations of Compound 1, with the fourth order polynomial model.

For non-human primate pharmacodynamics analyses, naïve cynomolgus monkeys (as described in NHP PK) were IV administered with a single dose of Compound 1 at 0.5 or 1 mg/kg. 1 ml of whole blood was collected via venipuncture in K2EDTA tubes (BD, #368841 before dosing, and at 15 min, and 2, 6, and 24 hrs, and day 2, day 5, day 6, day 8 timepoints. PD assessment for Vβ6+/Vβ10+in cynomolgus monkeys was done by flow analysis. Briefly, 50 μL of whole blood (for cell surface staining panel) were processed for flow cytometry analysis. The list of antibodies used were as follows for cell surface staining panel: FITC anti-CD14 (clone M5E2, Biolegend, #301804), PE anti-CD19 (clone J3119, Beckman Coulter, #1M1285U), AF700 anti-CD2 (clone RPA-210, Biolegend, #300238), PE-Cy7 anti-NKG2A (clone Z199, Beckman Coulter, #B10246), BV510 anti-CD25 (clone M-A251, Biolegend, #356120). PerCP-Vio 700 anti-CD3 (clone 10D12, Miltenyi, #130-120-731), BV650 anti-CD8 (clone RPA-T8, BD Bioscience, #563821). BV711 anti-CD4 (clone L200, BD Bioscience, #563913), AF647 anti-Vβ6/Vβ10 (produced at Marengo Therapeutics). Vβ6+/Vβ10+ T cells were gated from single cells, live cells, CD14-NKG2A−, and CD2+CD3+. 95 μL of whole blood (for intracellular staining panel) were processed in a separate panel for assessing pSTAT5 (in CD8+ T cells) and Tregs. The list of antibodies used were as follows for intracellular staining panel: BV711 anti-CD4 (clone L200, BD Bioscience, #563913), PE anti-CD25 (clone M-A251, Biolegend, #356104), Pacific Blue anti-CD8 (clone RPA-T8, BD Bioscience, #558207), APC-Cy7 anti-CD3 (clone SP34-2, BD Bioscience. #557757). PE-Cy7 anti-NKG2A (clone Z199, Beckman Coulter, #B10246), AF488 p-STAT5 (clone 47/STAT5, BD Bioscience, #612598), AF647 FoxP3 (clone 259D, Biolegend, #320214), pSTAT5 CD8+ T cells were gated from single cells, live cells, NKG2A-CD3+CD4−, and CD8+. Tregs were gated similarly with CD4+ and FoxP3+. Stained samples were analyzed utilizing the LSRFortessa II Flow Cytometer (BD Biosciences). Blood samples were also tested for blood biochemistry analysis using the Olympus Cobas 6000 (Roche Diagnostics), and blood hematology analysis using the Avdia 120 Analyzer (Siemens).

For ex-vivo cytotoxicity evaluations of Compound 1, the high content analysis assay using confocal microscopy was developed with three colors. TILs and PDX-O tumor cells are labeled with distinct dyes prior to co-culture, and a dead cell detection dye is added to the co-culture at the study endpoint. On Day 3, cryopreserved PDX fragments were thawed, counted, and stained with CellTracker Deep Red (CTDR). Stained PDX-fragments were washed and re-suspended in PDX-organoid media and plated at a density of ˜5000 cells in 50 μl organoid media/well in a 96-well plate ultra-low attachment round bottom plate. Organoids were allowed to form over a period of 3-days. After three days, on Day 0, PDX-O matched TILs were stained with Cell Trace Violet (CTV). Stained TILs were washed, re-suspended in TIL media, and co-cultured with matched PDX-O at a density of ˜25,000 cells/well (in 50 μL TIL media) in the appropriate wells. On the same day (Day 0), Compound 1 at 3 μg/ml and pembrolizumab at 10 g/ml (in additional 50 μL PDX-O media) were administered in quadruplicates. At the study endpoint, on Day 5, the NucGreen® Dead 488 probe was added directly to the wells to stain dead cells. Image z-stacks were captured approximately 3 hours after dead cell stain at 4× magnification using CellInsight CX7 LZR HCA instrument. Images were collected using filter sets appropriate for each marker and analyzed using HCS Cellomics Studio Software (per well). For all the models examined, PDX-O cytotoxicity was quantified through examination of the organoid size and reported as relative organoid size after normalized to isotype molecule.

Fresh tumor samples were collected from patients with a range of solid tumors from European clinical centers following local ethical approvals (19.11.1461-GI-M). Tumors and matched PBMC were collected from 55 donors immediately after surgery, processed for cell dissociation, stained for expression of V6-5 TCRs using a labelled antibody (clone IM2292, Beckman Coulter), and analyzed by flow cytometry.

Human PBMCs isolated from healthy donors were stimulated with serial dilutions of Compound 1 starting at 100 nM for 5 days with anti-Vβ6/V0p10 monovalent Fab and anti-RSV Fab x IL-2 single-arm controls. T cell activation was quantified by CD25 expression. Counting Beads (Thermofisher, #C36950) were included for direct T cell quantitation. The kinetics of expansion of Vβ6/Vβ10 T cells was measured by flow cytometry by directly staining for Vβ6/Vβ10 T cells (using a biotinylated-anti-Vβ6/Vβ10) over 8 days of Compound 1 treatment.

C57BL/6 albino or BALB/c mice between 5-6 weeks of age were obtained from The Jackson Laboratory. BALB/c mice were implanted with 1×105RENCA, or 5×104CT26 or EMT6 tumor cells, and C57BL/6 mice were implanted with 5×104MC38, or 2×104B16F10 or RM1 tumor cells by subcutaneous injection, with cells resuspended in PBS. Mice were randomized into treatment groups (n=8-10 mice per group) when tumors reached 80-150 mm3 and monitored daily for morbidity and mortality. Treatment antibodies were administered for 3-4 cycles by IP injection once/week. Tumor growth was monitored using calipers and calculated using the formula: tumor volume=0.52×(length)×(width)². Mice were euthanized when tumor volume reached 2000 mm3, or when body weight loss of >20% within 1-week was observed. Cured mice were monitored until day 100 and rechallenged with the same tumor cells up to 28 days post re-challenge: de novo challenge with CT26 cells was included in the opposite flank to serve as a control. In some re-challenge experiments, CD8+ T cells were depleted using an anti-mouse CD8a antibody (BioXCell, #BP0117) at 0.1 mg/kg on days −4 and −2 prior to rechallenge. Single arm control molecules were included as indicated. For Vβ13 T cell depletion studies, EMT6 bearing mice were treated with a depleting (Fc-enabled) anti-Vβ13 antibody at 1 mg/kg by IP injection every 4th day for 3 treatments starting 1-day before the first mSTAR treatment.

CD4+ and CD8+ TILs isolated from EMT6 mice treated with mSTAR or vehicle were loaded onto a 10× Chromium instrument to generate 3′gene expression libraries (10× Genomics, #1000268). Libraries were sequenced on NovaSeq to a minimum sequencing depth of 50,000 reads per cell. Cell Ranger v6.0.I was used to demultiplex FASTQ reads and align to the mm10-2020-A mouse transcriptome. Data quality was checked using FastQC and MultiQC v1.9. Data output from Cell Ranger was imported in R and processed with Seurat (v4.1.1) for downstream analyses.

Total RNA was extracted from murine TILs using Maxwell SimplyRNA Kit (Promega. #AS1390). Sequencing libraries were generated using SMARTer Human TCR a/pProfiling Kit (Takara, #635016), or SMARTer Mouse TCRα/pProfiling Kit (Takara, #634403). Final libraries were then pooled and sequenced on Illumina MiSeq. Data generated was demultiplexed and FastQC was performed after trimming. MiXCR pipeline tool was used to align sequence reads to germline segments of TRA, TRB, TRD and TRG genes and CDR3 sequences. TRBV genes are counted and represented as a percentage of frequency/abundance. Quantification of unique CDR3 clonal sizes were also grouped within each TRBV gene. CDR3 richness is calculated by the inverse Simpson's diversity index.

EMT6, CT26 and B16F10 cells were lysed with cell lysis buffer to harvest cell lysate proteins. Untreated tumor-free naïve splenocytes were used as antigen-presenting cells and were mixed with cell lysates for 30 mins at 37° C. to allow antigen presentation. Separately. T cells were purified by magnetic separation (Miltenyi, #130-095-130) from spleens of EMT6-tumor bearing mice treated with 0.5, 1, 1.5 mg/kg of mSTAR. The antigen presenting cell complex was cocultured with isolated T cells at a 2.5:1 ratio for 16 hours followed by the addition of Brefeldin A (5 μg/ml) for 4 hours. T cell responses to tumor-experienced T cells were quantified by intracellular staining for IFN-γ in Vβ13 CD8+ T cells.

For the cynomolgus studies, subjects were administered single 0.5, 1 or 1.5 mg/kg IV doses of Compound 1. For serum analysis, blood was collected in Serum Separation Tubes. Serum concentrations of Compound 1 were assessed using a bespoke MSD assay, cytokines were analyzed using a Luminex assay (Millipore, #PRCYTOMAG-40k), and sCD25 was analyzed using ELISA (R&D Systems, #DY223). For immunophenotyping analysis, blood was collected in K2EDTA tubes, stained with anti-Vβ6/Vβ10 TCR, anti-CD4, anti-CD8, and anti-FOXP3, then lysed and analyzed via flow cytometry.

The antitumor activity of Compound 1 and Pembrolizumab as a single agent was evaluated ex vivo in 4 human autologous patient organoids (Champions TumorGraft) models comprising donors CTG-3493 and CTG-3571 (non-small cell lung cancer), CTG-3629 (rectal cancer), and CTG-3631 (colorectal cancer)). The expansion of TILs and levels of viable and dead tumor cells was assessed over the 5-day culture by confocal high content image analysis, and tumor cell cytotoxicity established by changes in organoid size relative to isotype treatment. The antitumor activity of anti-RSV Fab x IL-2 was evaluated in the CTG-3571 patient organoid model. 1009711PBMCs from healthy donors from the NIH Clinical Center Blood Bank (NCT00001846) and a cervical cancer patient enrolled in a clinical trial prior to therapy (NCT03427411) were cryopreserved and stimulated with overlapping 15-mer peptides encoding HPV-16 E6 and E7 oncoproteins. DMSO or peptides encoding human leukocyte antigen served as negative controls. Prior to peptide stimulation, cells were treated for 1 hr with 1 nM Compound 1, an isotype control, or anti-V1p6N10 monovalent Fab. The absolute number of CD4+ or CD8+ T lymphocytes producing cytokine (IFN-γ, TNF-α, IL-2) or positive for a degranulation marker (CD107a) at the end of expansion was calculated per 1×106cells at the start of the assay. Multifunctional T cells expressing two or more of IFN-γ, TNF-α, IL-2, or CD107a, were quantified.

Unless otherwise noted, data presented in this Example were analyzed in GraphPad Prism 9 software. Data with two groups were analyzed by unpaired students t-Test or Wilcoxon match-pairs signed rank test. Data with multiple groups were analyzed by One-way ANOVA with Brown-Forsythe and Welch tests. Survival curves were analyzed by log-rank (Mantel-Cox) test. Data shown as mean±SEM. Significance shown as ****P-0.0001, ***P<0.001, **P<0.01, *P<0.05, or ns=not significant. Symbol representations are defined in legends. n represents number of animals or sample sizes.

### Results

The objective of this study was to develop a bispecific antibody fusion molecule that selectively targets certain TCR β chains and induces T cell activation, expansion, and antitumor activity.

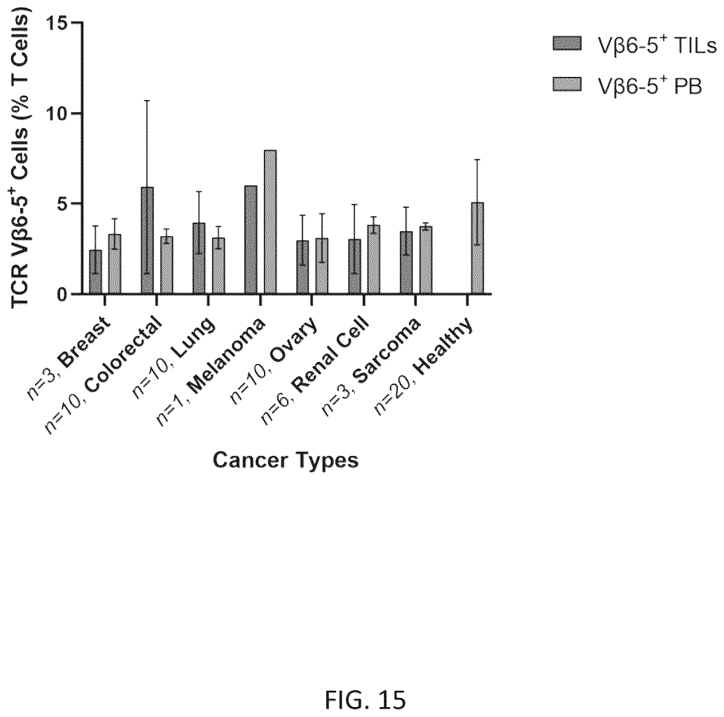

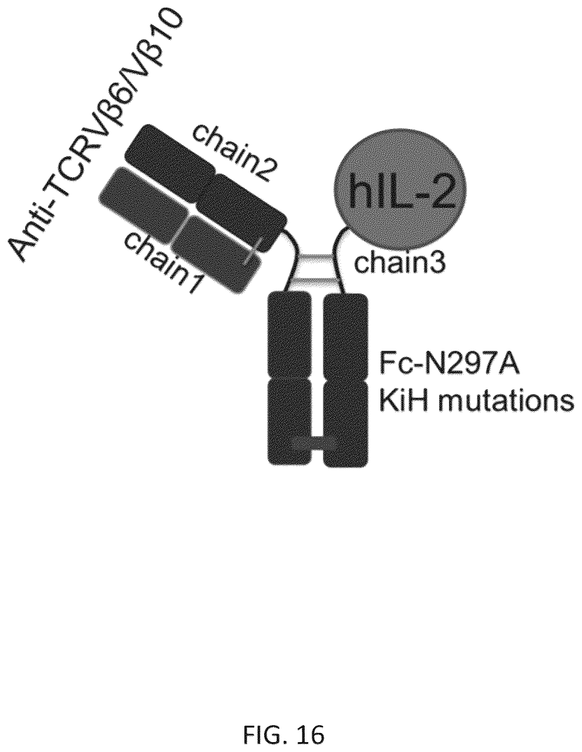

Vβ6 TCR T cell subset was selected since it is enriched in TILs relative to other Vβ subsets and is common across all cancers. To confirm this finding at the protein level, the prevalence of Vβ6-5 TCRs (the most common member of the Vβ6 family) was assessed in TILs from primary tissue collected from patients with a range of solid tumors. As measured by flow cytometry, Vβ6-5 T cells comprised between 2-8% of TILs across tested tumors, with similar frequencies observed in donor-matched peripheral blood mononuclear cells (PBMCs), and healthy donor PBMCs (FIG. 15). Based on this finding, Compound 1 was designed as a bifunctional antibody-fusion molecule comprising an affinity-matured anti-Vβ6-5 Fab, and a native IL-2 molecule fused by its C-terminus to the Fc domain (FIG. 16). The IgG1 Fc domain of Compound 1 contains knob-in-hole mutations to promote Fc heterodimerization and an N297A mutation to abrogate effector functions. Compound 1 bound with high affinity (Kr) to purified human Vβ6-5 (1.7 nM), IL2Ra (45 nM), IL2Rβγ (3.8 nM), and the high affinity trimeric IL2R (0.1 nM), which is similar to the affinity of both recombinant IL-2 (rIL-2) and the “in-format” anti-RSV-IL-2 Fab control molecule (IL-2 control) for the IL2R subunit chains (Table S1).

TABLE S1
Equilibrium dissociation constants of Compound 1
and rhIL2 for human and cynomolgus TCRVβ and IL2R
proteins as determined by surface plasmon resonance.

| Antigen | Compound 1 (K_{D}) | rhIL2 (K_{D}) |
|---|---|---|
| Human Vβ6-5 | 1.7 nM | N/A |
| Cynomolgus Vβ6-2 | 4.9 nM | N/A |
| Human IL2Rα | 45 nM | 72 nM |
| Cynomolgus IL2Rα | 48 nM | — |
| Human IL2Rβγ | 3.8 nM | 5 nM |
| Cynomolgus IL2Rβγ | 3.3 nM | — |
| Human IL2R Trimer | 0.1 nM | 0.8 nM |
| Human Vβ10-2 | 460 nM | N/A |
| Human Vβ10-3 | 685 nM | N/A |

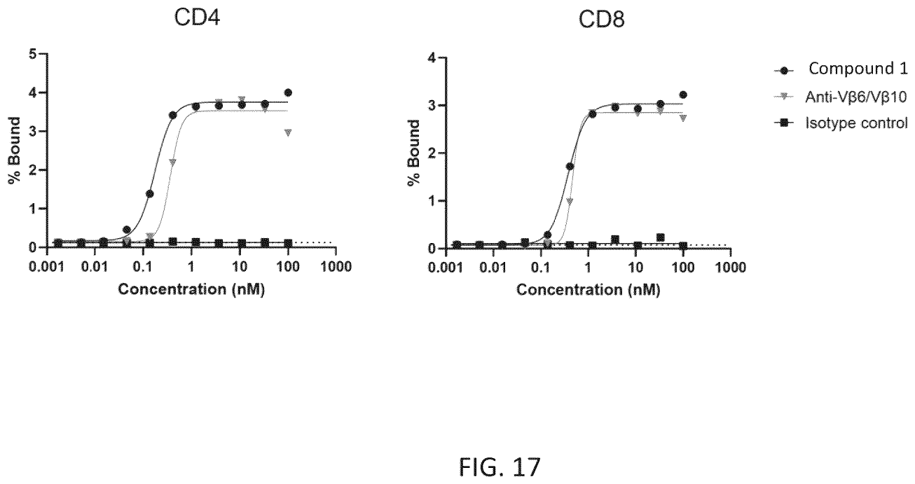

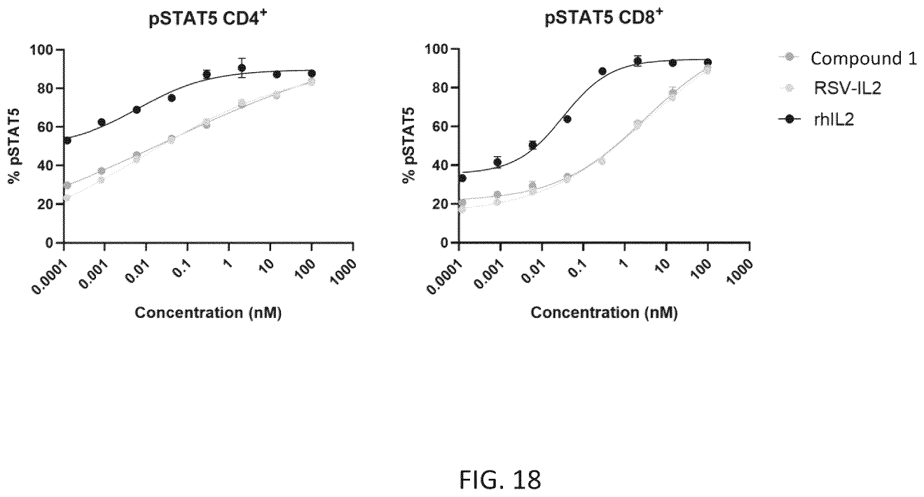

Compound 1 bound to CD4+ and CD8+ T cells with similar affinities, with a half maximal effective concentration (EC₅₀) of 0.21+/−0.03 nM in CD4+ T cells and 0.52+/−0.2 nM in CD8+ T cells (FIG. 17). In a cellular assay of IL-2 bioactivity using the detection of pSTAT5 (low expression levels of IL-2R on naïve T cells prohibits accurate cellular IL-2 binding studies), both Compound 1 and the IL-2 control molecule showed significantly attenuated (˜100-fold lower) IL-2 bioactivity compared with rIL-2 (FIG. 18).

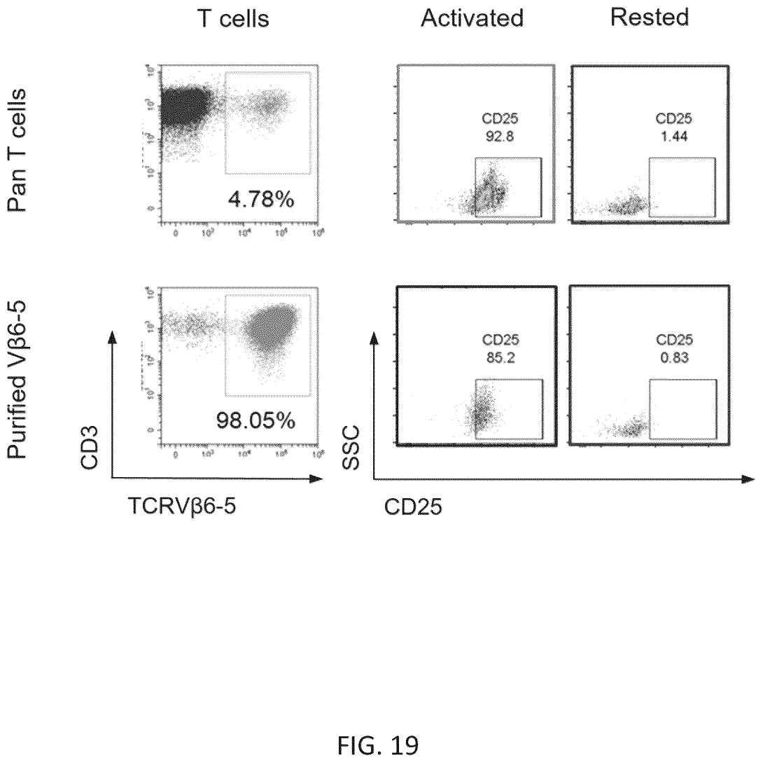

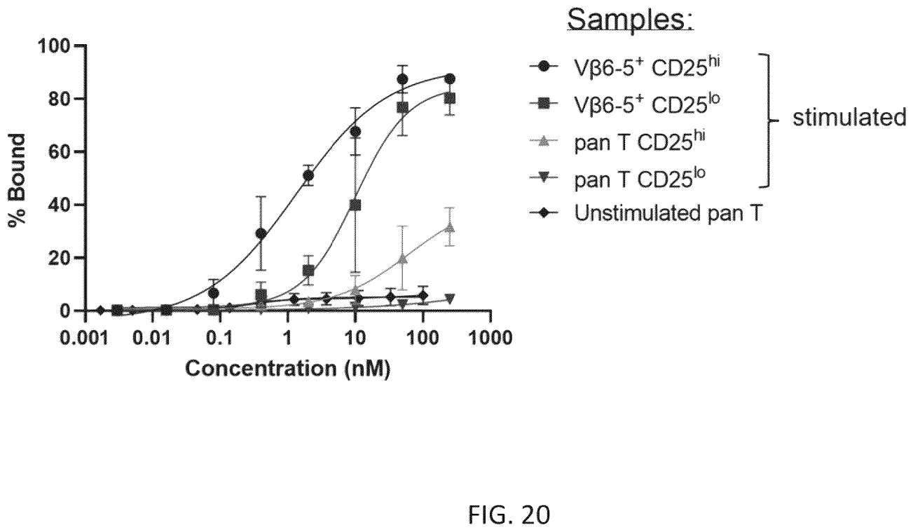

To further explore the mechanism of binding of Compound 1, four human T cell populations were created in vitro using a combination of flow cytometry sorting methods and cycles of T cell stimulation and/or resting to generate populations of cells with contrasting levels of Compound 1 target expression corresponding to the extremes of possible Vβ6-5 T cell and CD25+ T cell frequencies (FIG. 19). Binding of Compound 1, at a single cell level, over a broad concentration range was then measured in these manipulated T cell populations using flow cytometry. In untreated T cells isolated from healthy human donors. Compound 1 bound to 3-5% of T cells over a pharmacologically relevant concentration range, reflecting binding to the expected proportion of V6-5 T cells in a physiologically normal state (FIG. 20). In manipulated T cell populations comprising 98% Vβ6-5 T cells, Compound 1 bound to both CD25low and CD25high cells, with higher affinity binding to CD25high Vβ6-5 T cells (1.3 nM) compared with CD25low (10 nM) Vβ6-5 T cells. In unsorted, in vitro stimulated T cells comprising 3-5% Vβ6-5 T cells and >98% CD25high T cells (an uncommon state for the human T cell compartment), Compound 1 bound with much lower affinity (62 nM).

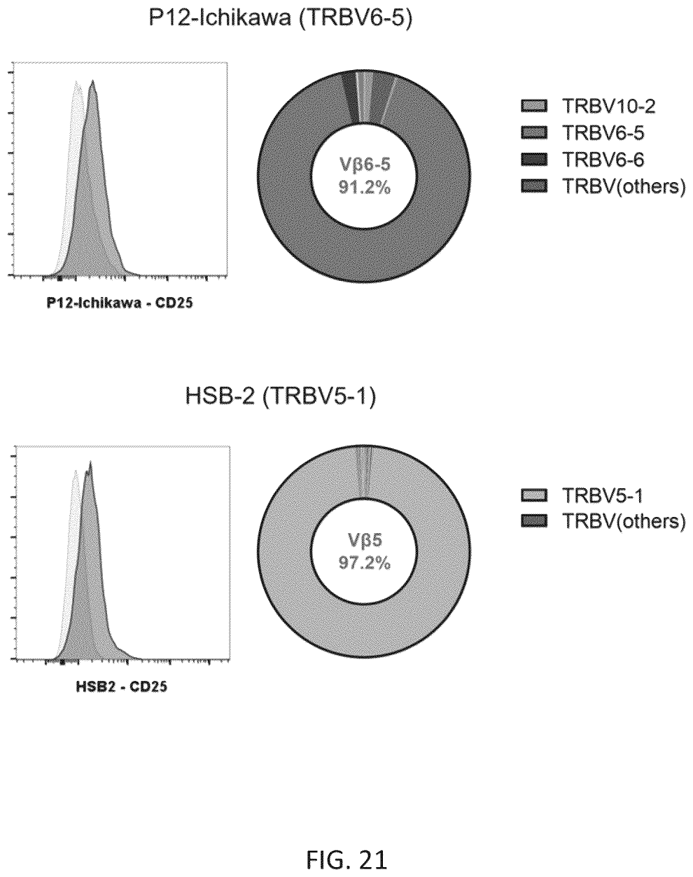

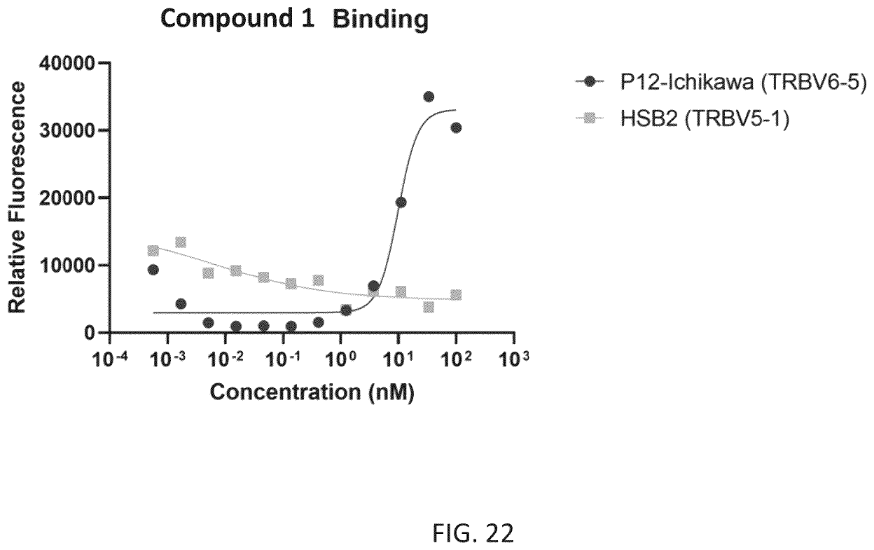

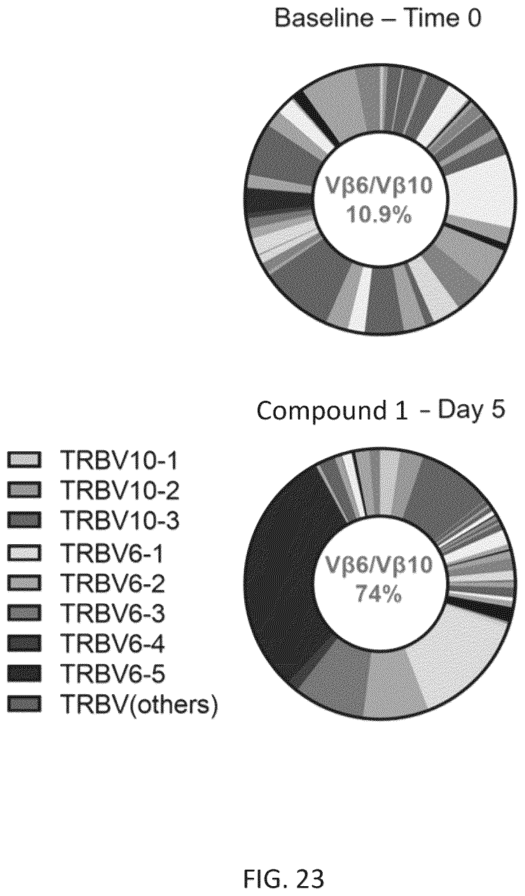

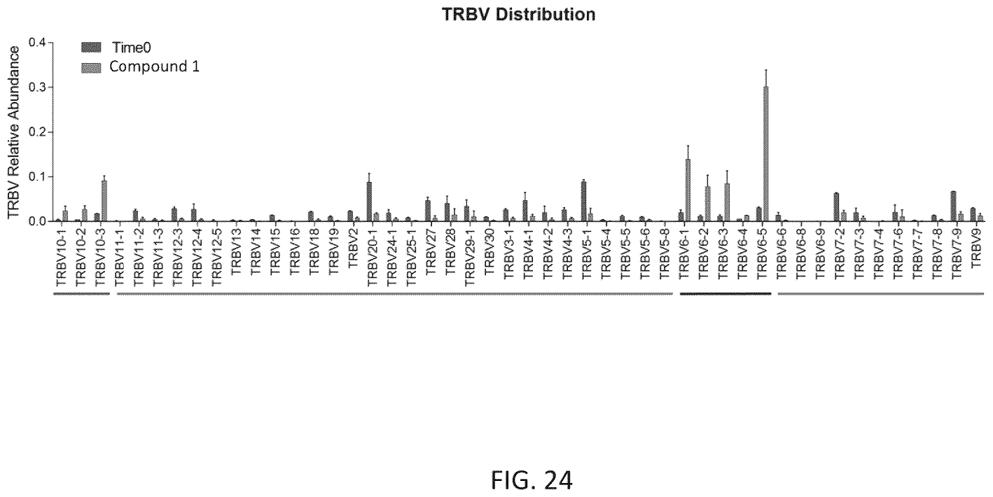

The binding of Compound 1 was evaluated to established T lymphocyte cell lines that exclusively co-expressed CD25 and either Vβ6-5 (P12-Ichikawa) or an unrelated Vβ chain TCR (HSB-2 cells-expressing Vβ5-1) (FIG. 21). Compound 1 bound only to P12-Ichikawa cells whereas HSB-2 cells showed minimal to no binding (FIG. 22). 1009791To assess the functional selectivity of Compound 1, PBMCs from healthy donors were stimulated in vitro with Compound land the relative abundance of VβT cell subsets assessed by TCR sequencing. Among the 48 known germline-encoded Vβchains, Compound 1 promoted the expansion of predominantly Vβ6 T cell subsets (i.e., TRBV6-1, 6-2, 6-3, 6-4, 6-5): some limited activation and expansion of T cells expressing Vβ10 transcripts (TRBV10-1, 10-2, 10-3) was also observed (FIGS. 23-24), reflecting the close sequence homology between these two V gene families. Compound 1 predominantly expanded Vβ6 T cells, with this subset comprising >60% of T cells after Compound 1 stimulation.

In healthy donor PBMCs, Vβ6 T cells comprised on average 5.2% of CD4+ and 6.8% of CD8+ T cells (Table S2). A comparison of the binding affinities of Compound 1 with the single arm control molecules (i.e., an anti-Vβ6-5 monovalent antibody, and an “in-format” IL-2 control comprising an unrelated anti-RSV Fab fused to IL-2) identified that the binding of Compound 1 was mainly driven by the anti-Vβ6-5 Fab.

TABLE S2
Frequency of Compound 1 binding in T cell subsets from healthy donors.

| PBMC Subset | Average (%) | Median | StDev | Range | n |
|---|---|---|---|---|---|
| CD3 | 6.10 | 5.29 | 2.95 | 3.32-14.15 | 26 |
| CD4 | 5.22 | 4.69 | 1.67 | 3.18-10.23 | 26 |
| CD8 | 6.82 | 4.61 | 5.33 | 2.35-22.72 | 26 |
| FoxP3⁺ Treg | 5.59 | 4.05 | 4.79 | 2.45-17.2 | 8 |

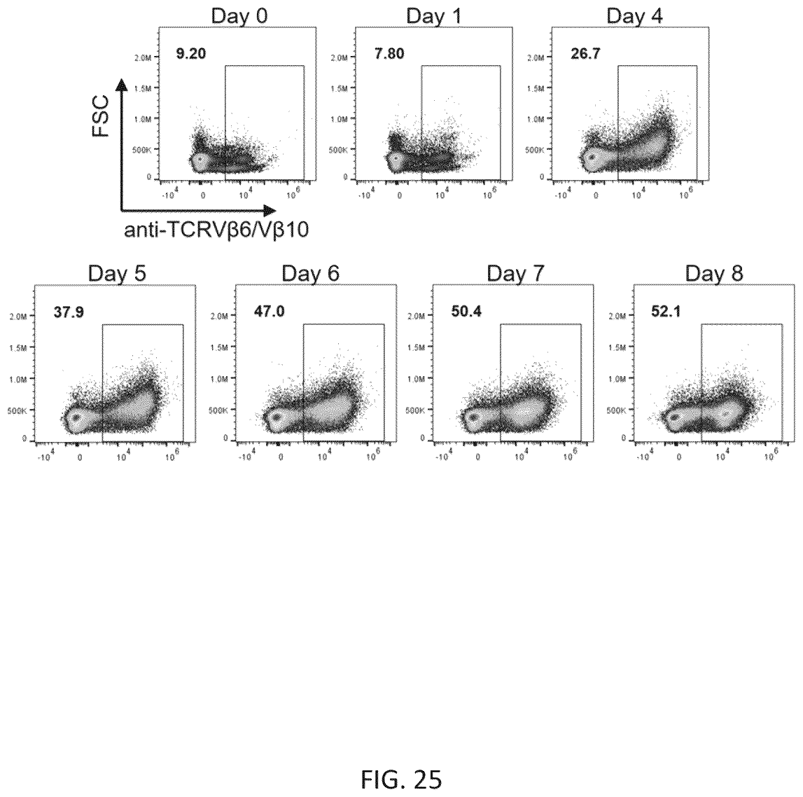

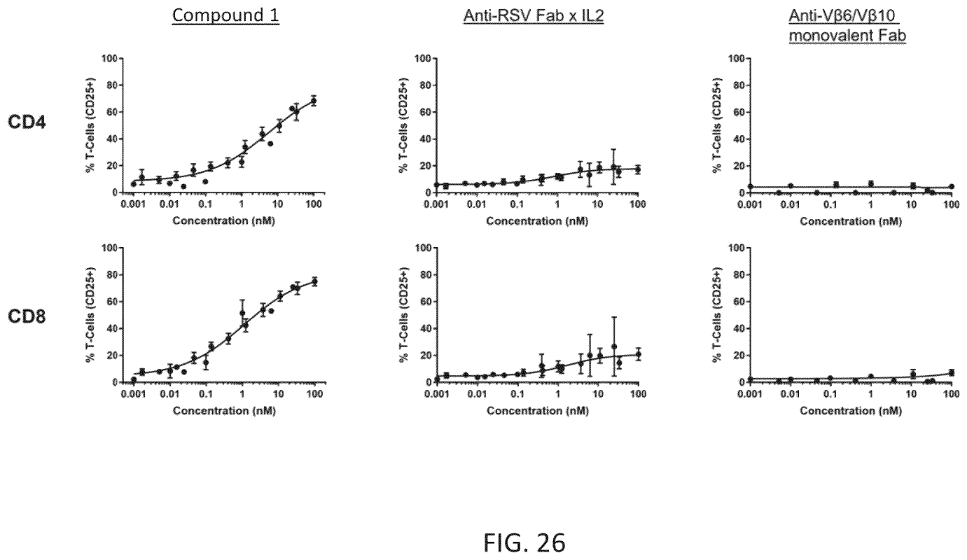

The kinetics of in vitro human Vβ6/Vβ10 T cell expansion by Compound 1 was investigated by enumerating Vβ6/Vβ10 T cells using flow cytometry. As shown in FIG. 25, Vβ6/Vβ10 T cells incrementally increased over time with frequencies rising from a baseline of 9% of T cells to 27%, 38%, 47%, 50%, and 52% on days 4, 5, 6, 7 and 8 post stimulation. The magnitude of in vitro activation of both CD4+ and CD8+T cells by Compound 1 was concentration-dependent (FIG. 26), albeit differences in EC₅₀ values (5.16+/−6.04 nM for CD4+ T cells and 1.98+/−1.81 nM for CD8+ T cells) suggest preferential activation of CD8+ over CD4+ T cells (Table S3).

TABLE S3
Potency of the activation of CD4+ and CD8+ T cells by
Compound 1 as determined by CD25 expression.

| T cell | | | | EC50 | |
|---|---|---|---|---|---|
| Subset | Constructs | EC50 (nM) | StDev (nM) | Range (nM) | n |
| CD4 | Compound 1 | 5.16 | 6.04 | 0.07-22.2 | 20 |
| CD4 | Anti-VB6/VB10 | N/A | N/A | N/A | 5 |
| CD4 | RSV-IL-2 | N/A | N/A | N/A | 6 |
| CD8 | Compound 1 | 1.98 | 1.81 | 0.07-5.36 | 20 |
| CD8 | Anti-VB6/VB10 | N/A | N/A | N/A | 5 |
| CD8 | RSV-IL-2 | N/A | N/A | N/A | 6 |

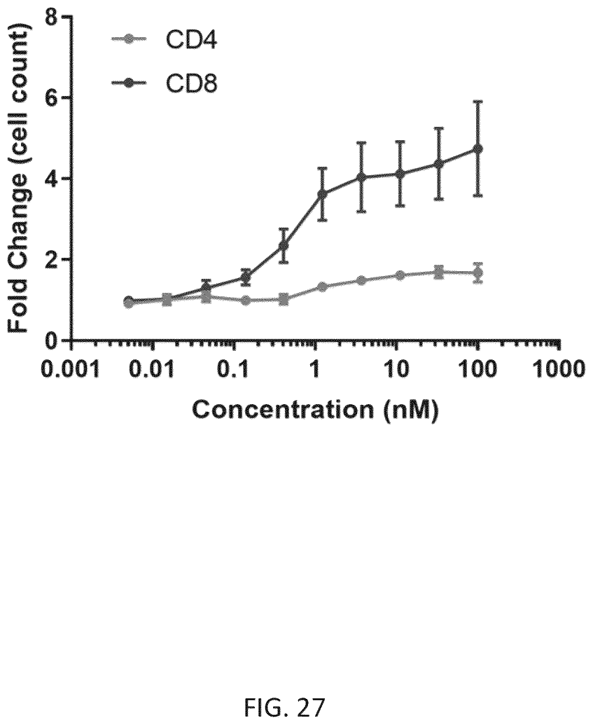

Single arm controls showed minimal to no activation of T cells. To further investigate differential in vitro expansion of human CD8+ versus CD4+ T cells, cell counts normalized to unstimulated samples were compared from the same donor. Compound 1 increased the number of CD8+ T cells to a 3-fold greater extent than CD4+ T cells, although the magnitude of this effect varied across donors (FIG. 27). Thus, although both CD4+ and CD8+ T cells are activated by Compound 1, the expansion of CD8+ T cells was more pronounced.

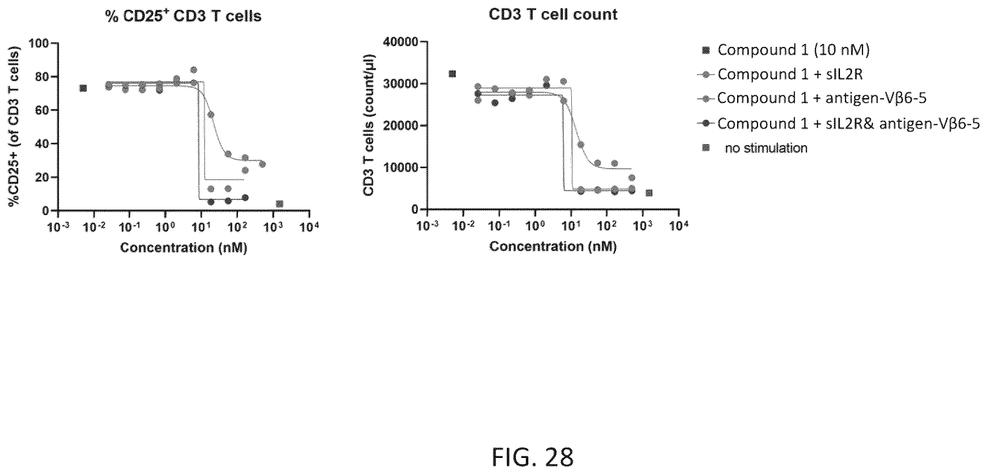

To explore whether this expansion and activation of T cells is dependent on engagement of both arms of Compound 1, purified T cells were incubated for five days with a fixed concentration of Compound 1 (10 nM) and increasing amounts of competing soluble IL2R, Vβ6-5 antigen, or a mixture of both. The addition of these competitors elicited a dose dependent inhibition of T cell activation (as measured by acquisition of CD25 expression) and expansion (FIG. 28).

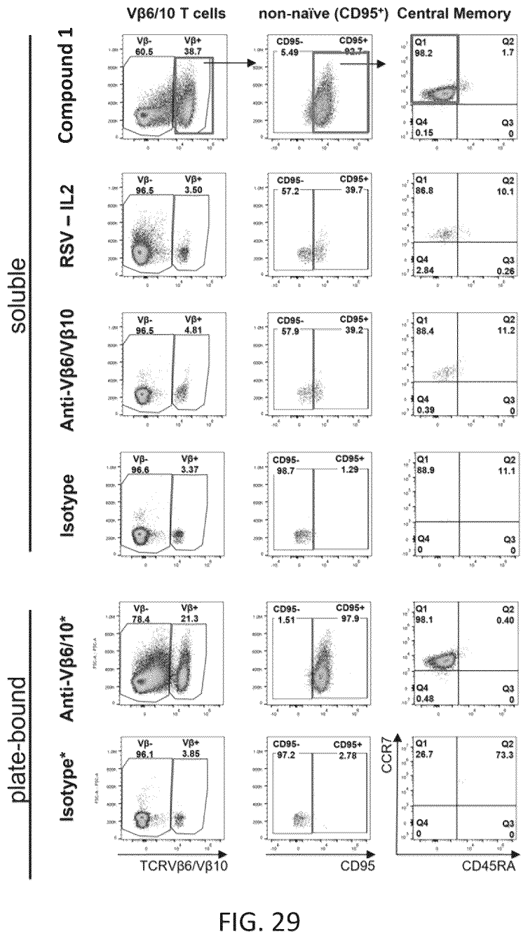

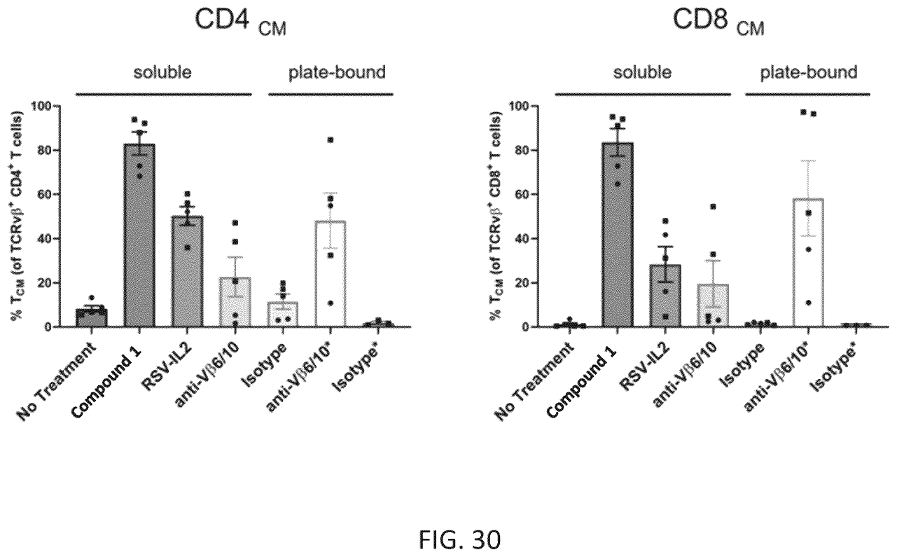

Treatment of human PBMCs or purified T cells with Compound 1 in solution induced a strong central memory (CD95+CCR7+CD45RA−) T cell phenotype (T_{CM}) in Vβ6/Vβ10 CD8+ T cells, that was not observed with single arm controls (FIG. 29). The combined action of the anti-Vβ6/Vβ10 Fab and the IL-2 moiety of Compound 1 was required to drive the activation of the Vβ6/Vβ10 TCR and TCM phenotype. This shift to a TCM phenotype is consistently observed in both CD4+ and CD8+ T cells across multiple donors (FIG. 30) and was not observed for control molecules (anti-Vβ6/Vβ10 monovalent Fab or IL-2 control) in solution.

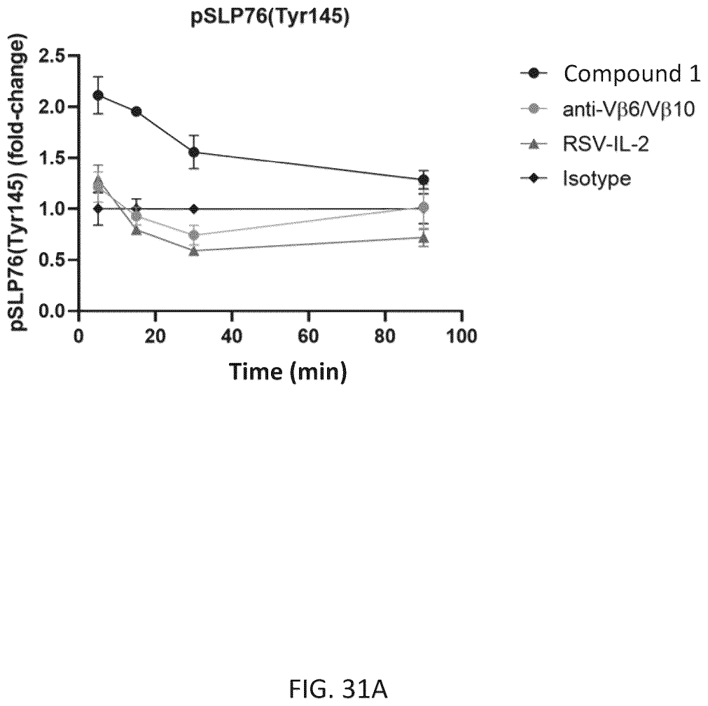

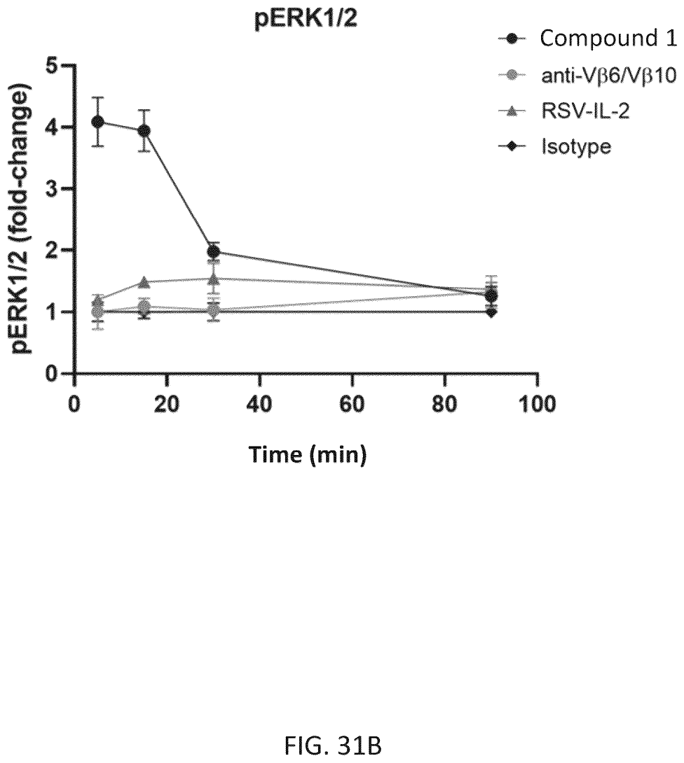

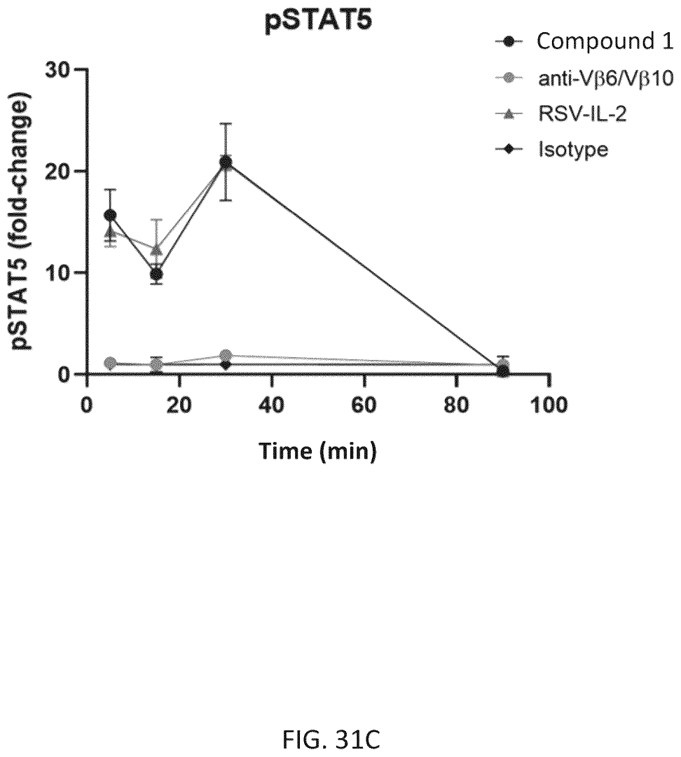

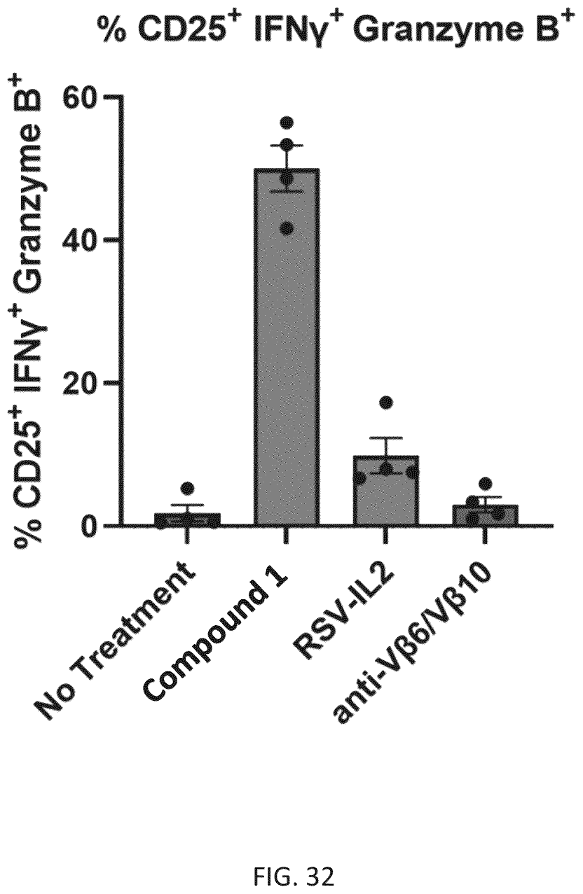

To investigate whether Compound 1 activated signaling pathways downstream of both the TCR and IL-2R, pERK, pSLP76 and pSTAT5 (proximal regulators of TCR and IL-2 signaling) were measured from cellular lysates. In Compound 1-stimulated human CD8+ T cells, pERK levels were increased 3.9-fold and pSLP76 (Tyr145) levels were 2.1-fold higher versus control molecules (FIGS. 31A-B). Plate bound anti-Vβ6/β10 Fab, also activated pERK and pSLP76 to similar levels as Compound 1 in solution, confirming crosslinking of Vβ6/Vβ10 TCRs with both molecules. Furthermore, pSTAT5 levels were increased up to 20-fold after treatment with Compound 1 and the single arm IL-2 control molecule, but not by the anti-Vβ6/Vβ10 monovalent antibody either in solution or plate bound (overlapping curves in FIG. 31C). Consistent with the impact of plate-bound anti-Vβ6/Vβ10 Fab on primary T cells, a marked upregulation of cells co-expressing CD25, intracellular Granzyme B and IFN-t in Compound 1-stimulated human CD8+ T cells was observed, demonstrating Compound 1 induced an atypical TCM phenotype (FIG. 32).

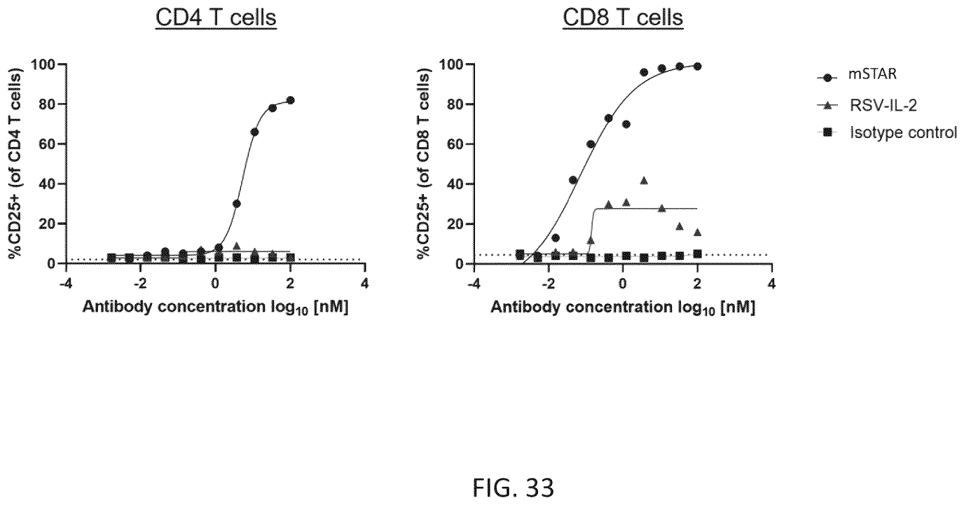

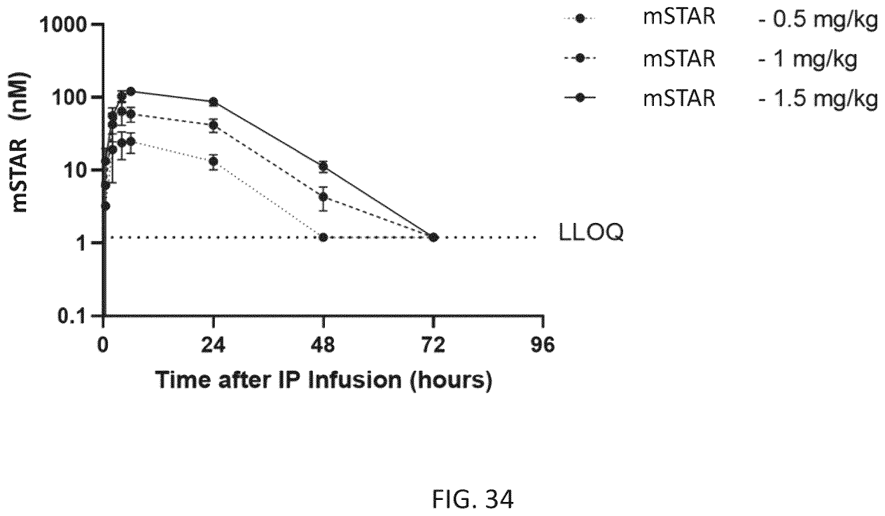

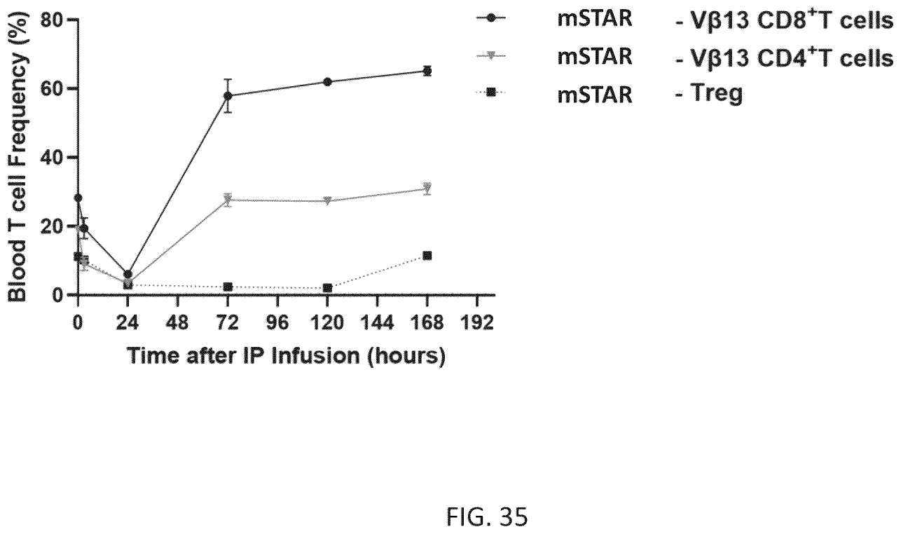

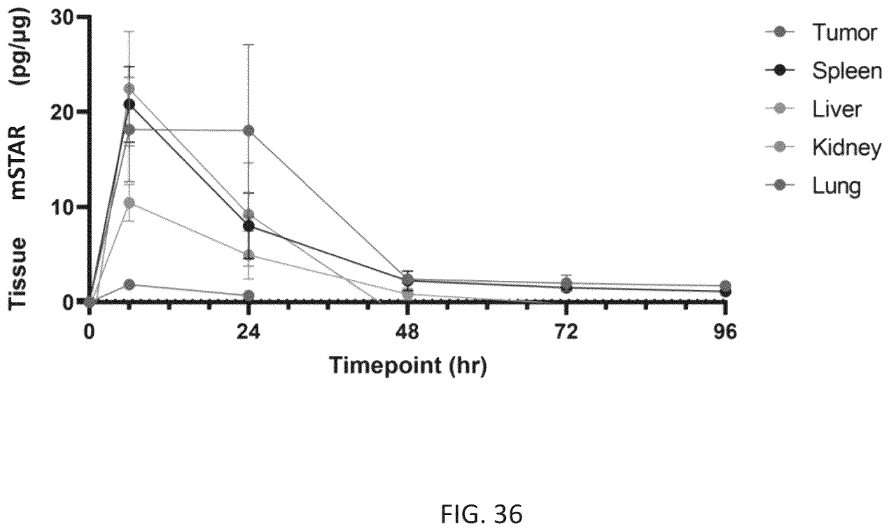

Since a homolog of the Vβ6 gene does not exist in mice, a surrogate molecule (mSTAR) comprising the same IL-2 antibody fusion design as Compound 1 and that targets and expands the most abundant murine Vβ T cell subset (Vβ13) was developed. Consistent with in vitro assessments of Compound 1 in human PBMCs, in vitro stimulation of mouse splenocytes resulted in the activation and expansion of both CD4+ and CD8+ Vβ13 T cells in a concentration dependent manner (FIG. 33), with similar potency to Compound 1 (EC₅₀12+/−13 nM for CD4+T cells and 0.4+/−0.6 nM for CD8+T cells). Following administration of a single intraperitoneal (IP) dose in BALB/c mice, mSTAR showed rapid clearance in serum (FIG. 34), yet induced robust expansion of Vβ13 CD8+T cells and to a lesser extent Vβ13 CD4+T cells in peripheral blood, without expanding T regulatory cells (Tregs) (FIG. 35). Further details of mSTAR pharmacokinetics are shown in Table S4. In BALB/c EMT6 tumor-bearing mice, Compound 1 distributed extensively into peripheral tissues including tumor, spleen, liver, kidney, and lung tissue up to 48 hours post-dosing (FIG. 36).

TABLE S4
Pharmacokinetic parameters of mSTAR in BALB/c mice.

| | Unit | 0.5 mg/kg | 1 mg/kg | 1.5 mg/kg |
|---|---|---|---|---|
| T ½ | h | 8.6 | 12.1 | 11.9 |
| Tmax | h | 6 | 4 | 6 |
| Cmax | nM | 24.8 | 64.3 | 121.0 |
| AUC 0-t | nM*h | 621.4 | 1731.6 | 3505.7 |

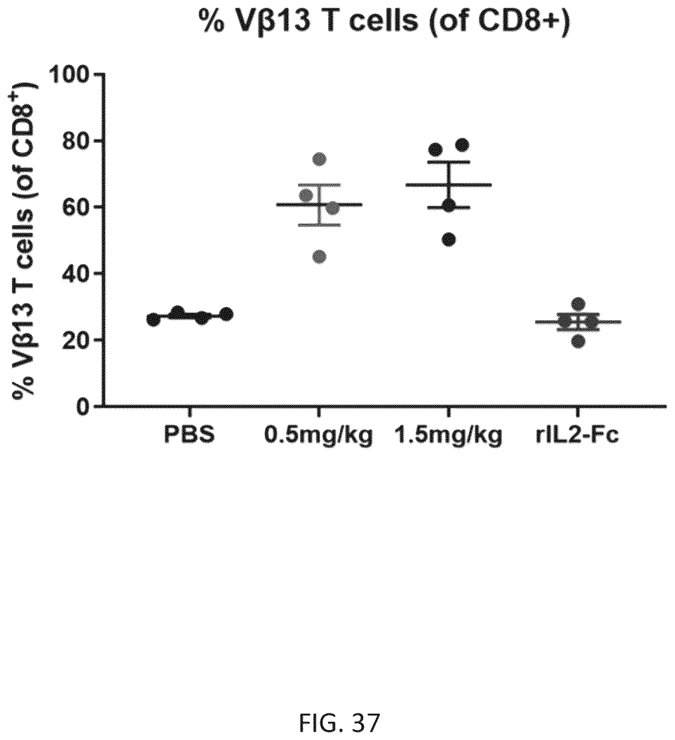

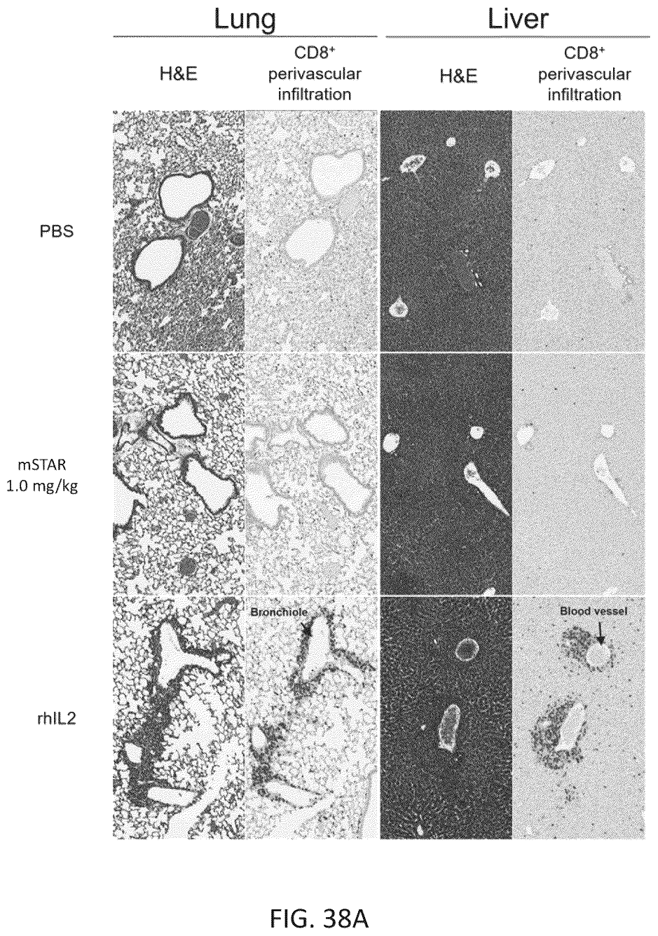

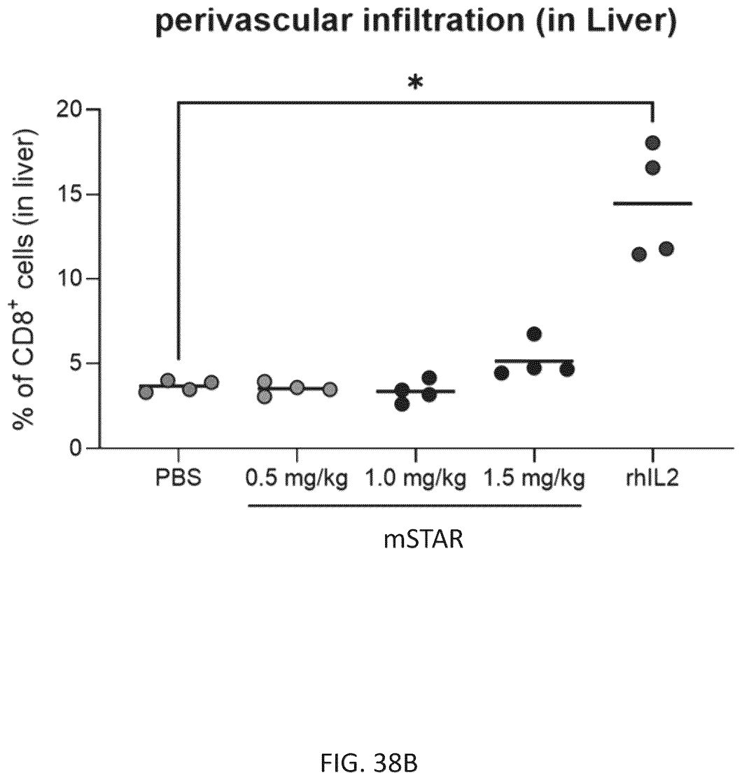

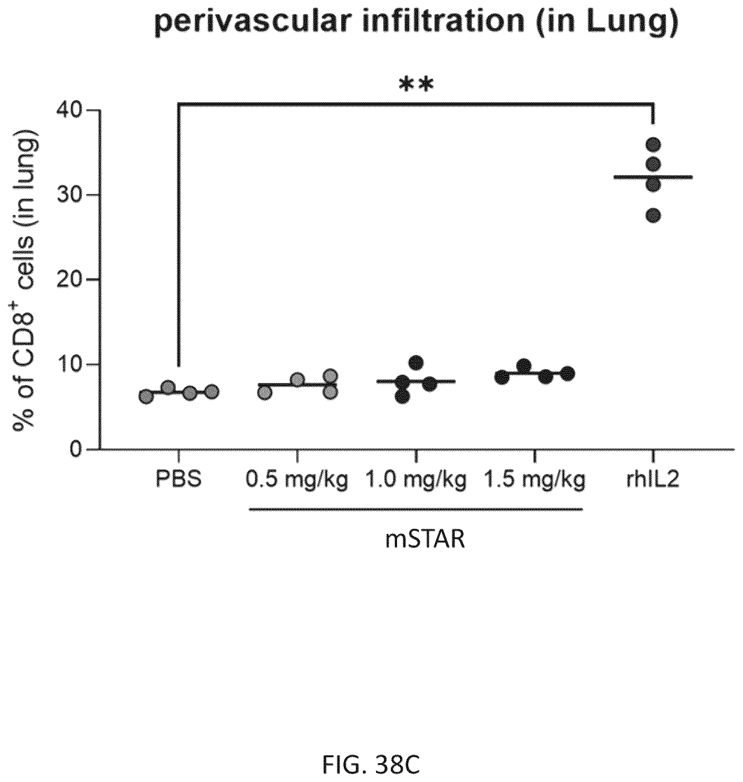

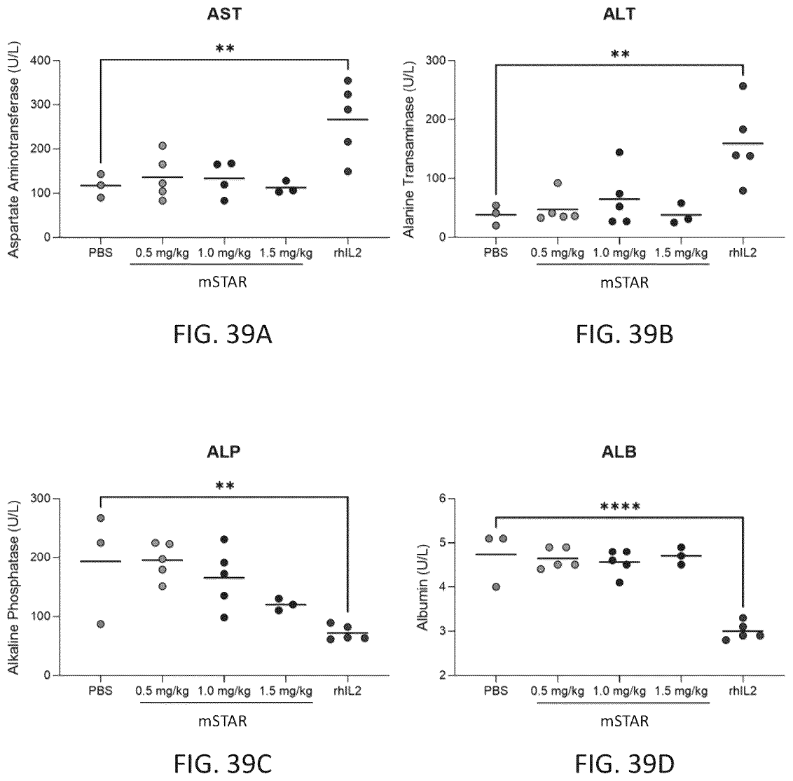

To investigate the potential for idiosyncratic IL-2-mediated toxicity such as vascular inflammation and injury, Balb/c mice were dosed with mSTAR (IP 0.5-1.5 mg/kg BIW), vehicle (PBS), or rhIL-2 at a dose regimen designed to re-capitulate high-dose Aldesleukin use in humans (a 9-day course of 8 mg/kg BID rhIL-2). In blood, targeted Vβ13 CD8+ T cells were expanded in mice dosed with mSTAR (FIG. 37) but not with vehicle or rhIL-2. In the lungs and liver of mice dosed with rhIL-2, pronounced perivascular leukocytic infiltration was observed surrounding sinusoidal liver vessels and pulmonary vessels (FIGS. 38A-C), consistent with early signs of vascular injury. Similar to the effects of Aldesleukin in humans, mice dosed with rhIL-2 also showed significant changes in liver enzyme markers of hepatic injury (FIG. 39A-D). In contrast, no perivascular inflammation or liver enzyme changes were observed in mice administered BIW mSTAR at doses up to and including 1.5 mg/kg.

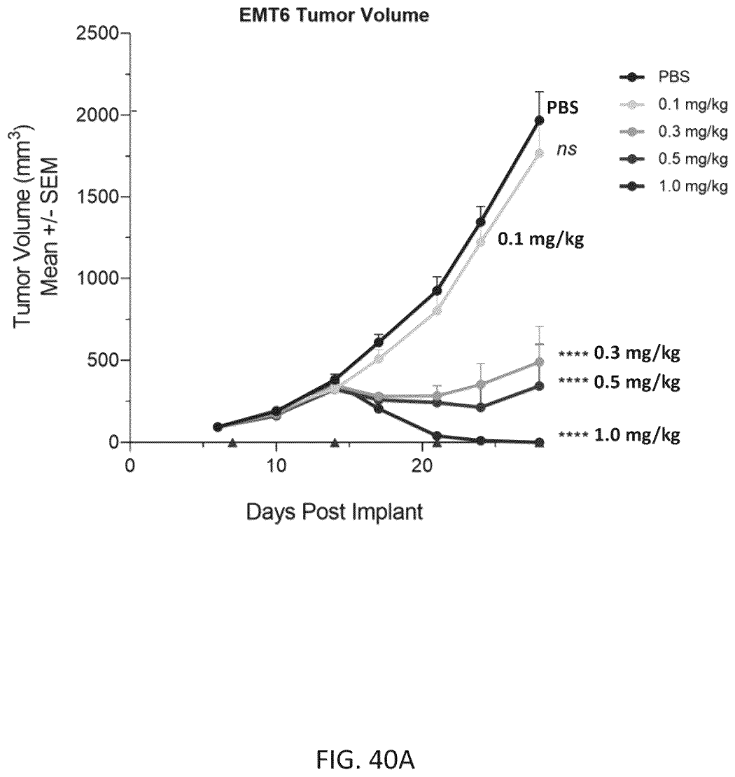

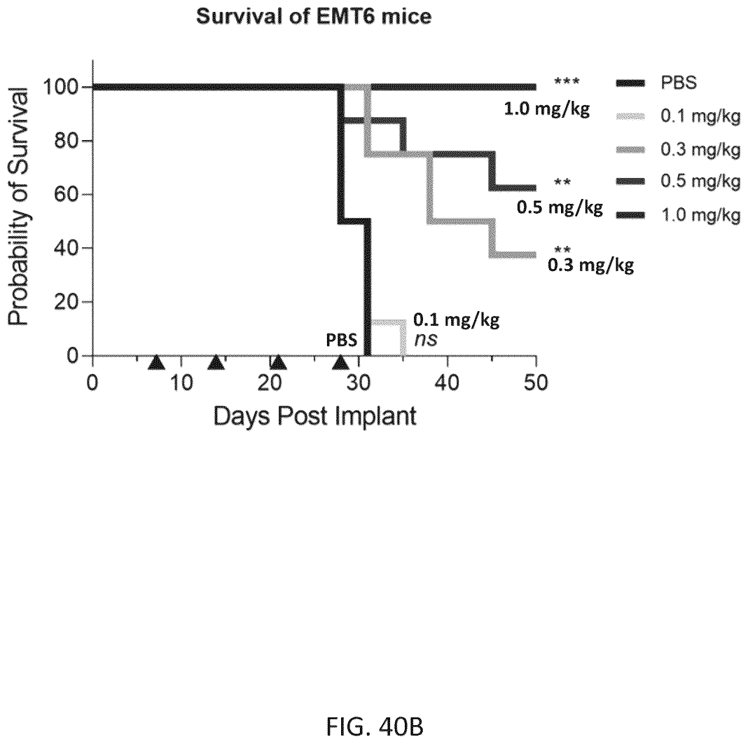

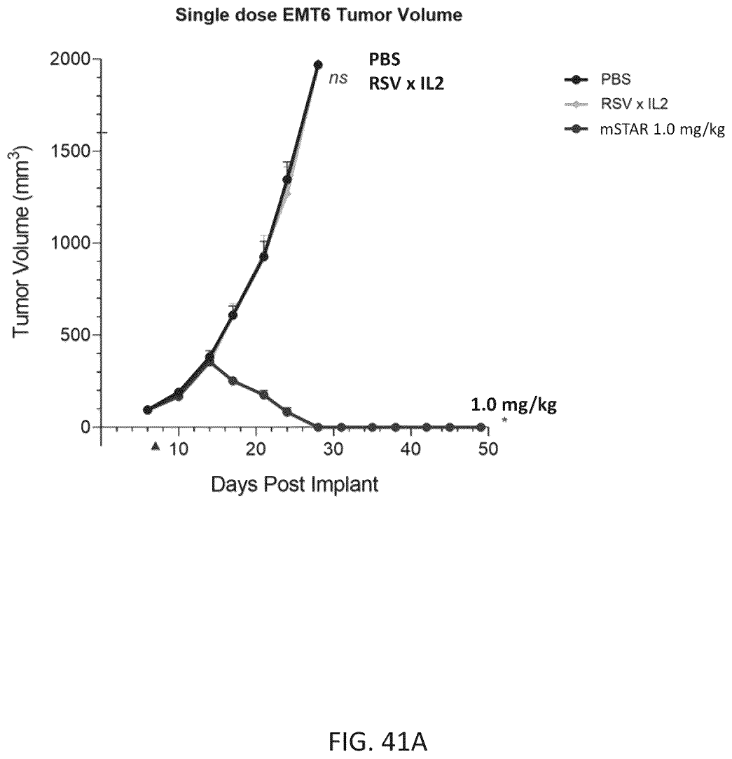

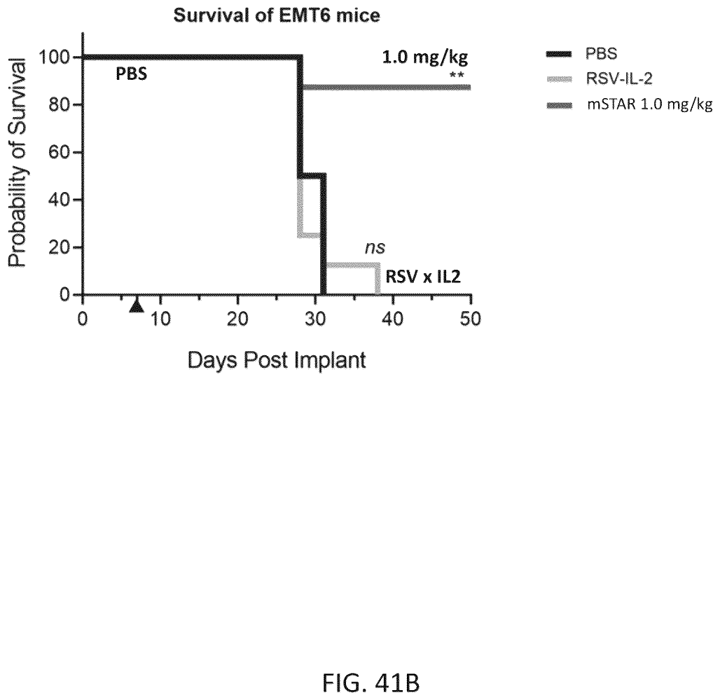

The antitumor activity of mSTAR was assessed in six murine syngeneic solid tumor models with differing levels of T cell infiltration, different tumor microenvironment (TME) conditions, and varying responsiveness to CPI therapy. In dose finding studies of mSTAR in the EMT6 breast cancer model, four consecutive 1 mg/kg doses given once weekly (QW) was determined as optimal in relation to anti-tumor activity (FIG. 40A-B); however, in subsequent scheduling studies, just a single 1 mg/kg dose of mSTAR was shown to elicit similar tumor regression (FIG. 41A-B).

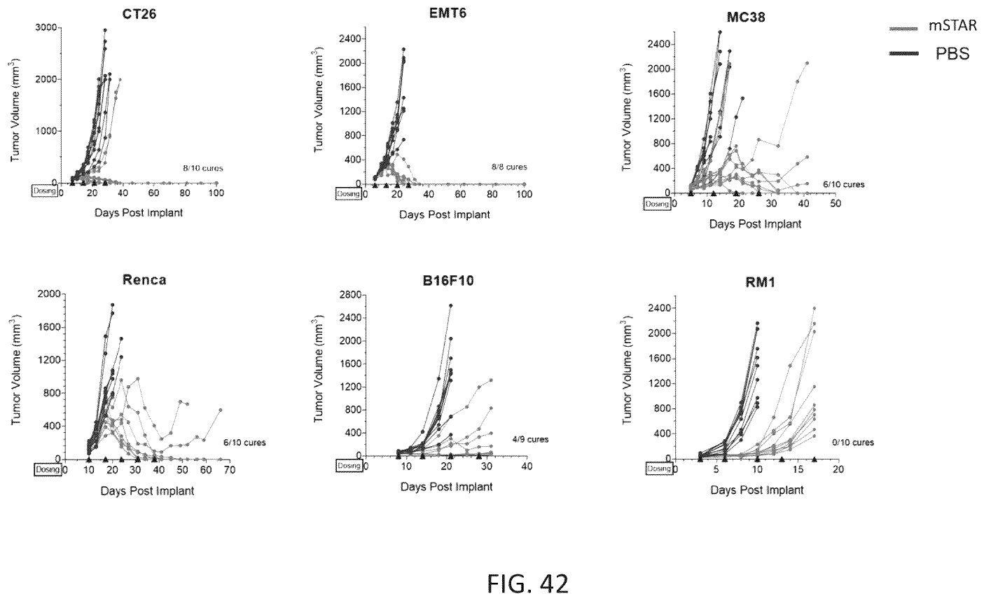

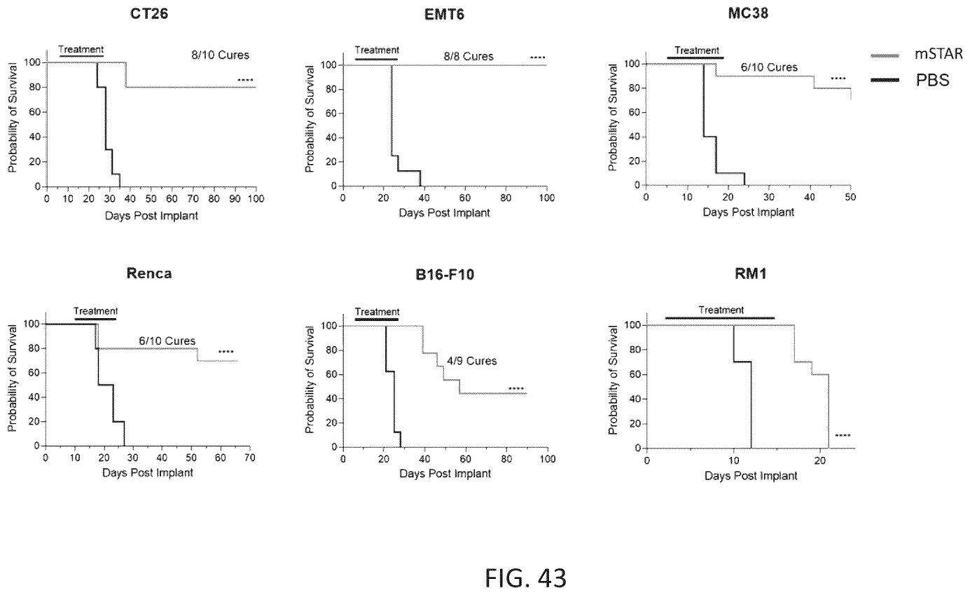

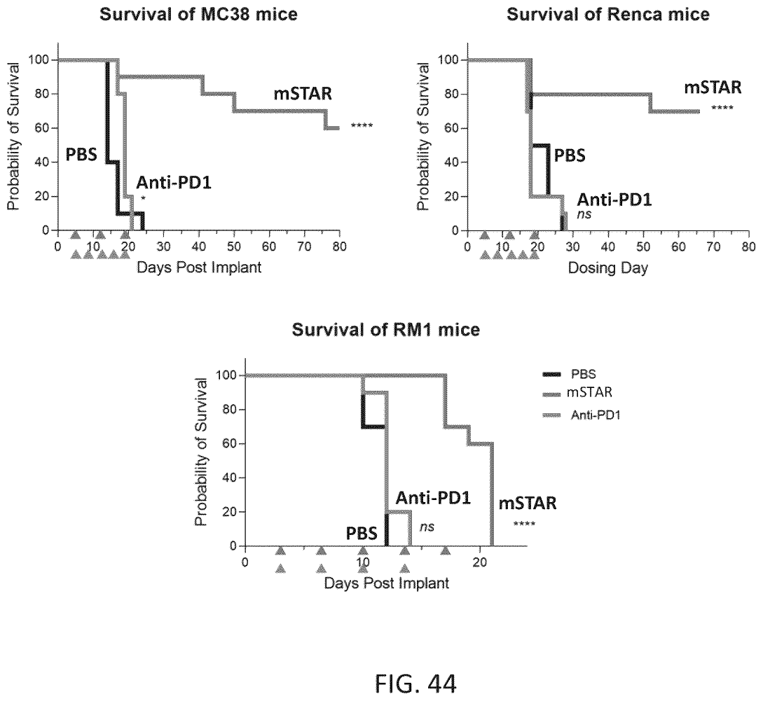

In mice treated with mSTAR as a single agent, significant tumor growth inhibition (TGI) was observed in all 6 syngeneic models with complete tumor regression in the EMT6 model. TGI compared to the PBS-treated group was calculated to be 98% in CT26 (day 24, a time point where the tumors from PBS group exceed 2000 mm3), 70% in MC38 (day 17), 66% in RENCA (day 20), 57% in RM1 (day 12), and 86% in the highly refractory B16F10 model (day 21) (FIG. 42). The anti-tumor activity of mSTAR also significantly increased survival in all 6 tumor models evaluated (FIG. 43). The activity of mSTAR was directly compared to a mouse anti-PD-1 antibody in a subset of CPI-refractory models (MC38, Renca, and RM1) and in all cases, mSTAR significantly improved survival compared to anti-PD-1 treatment (FIG. 44).

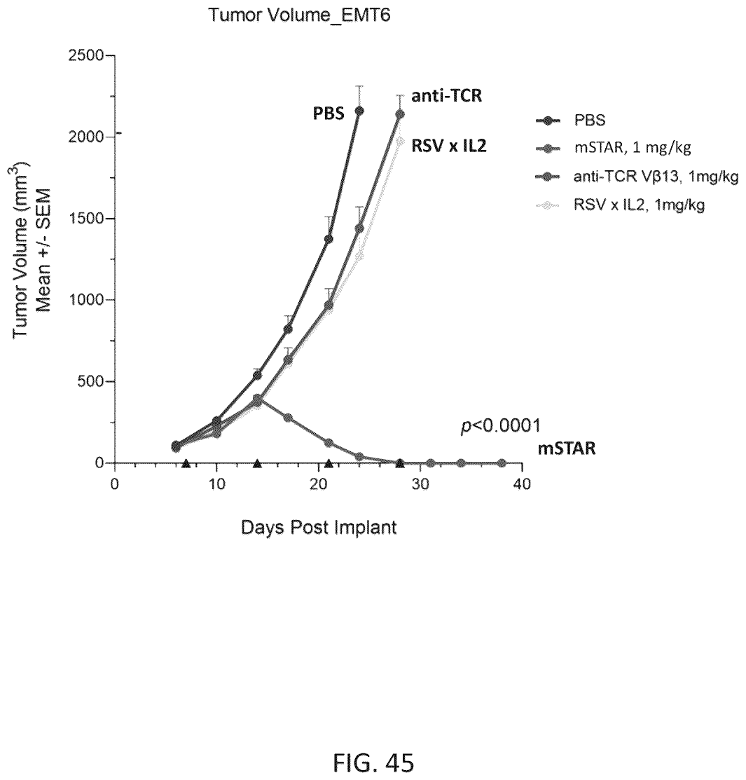

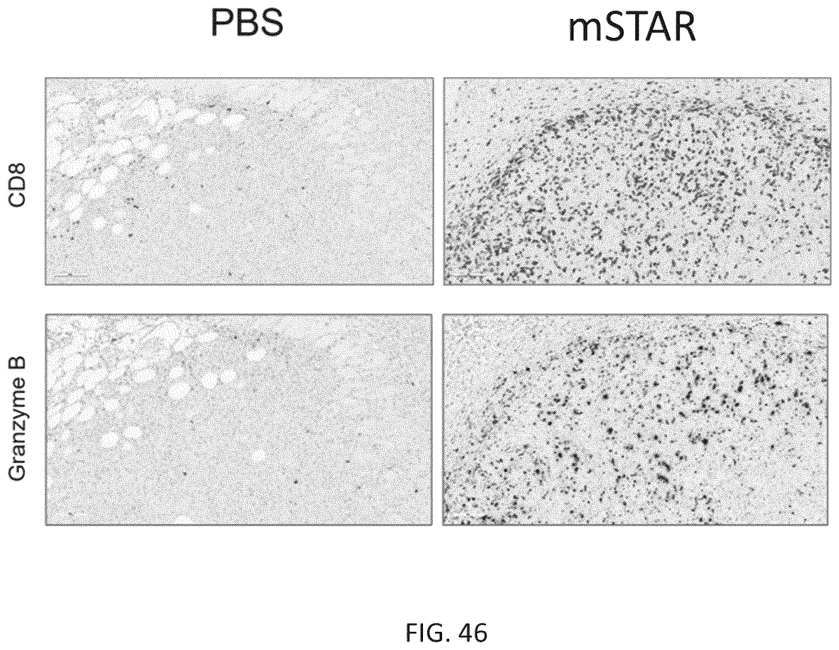

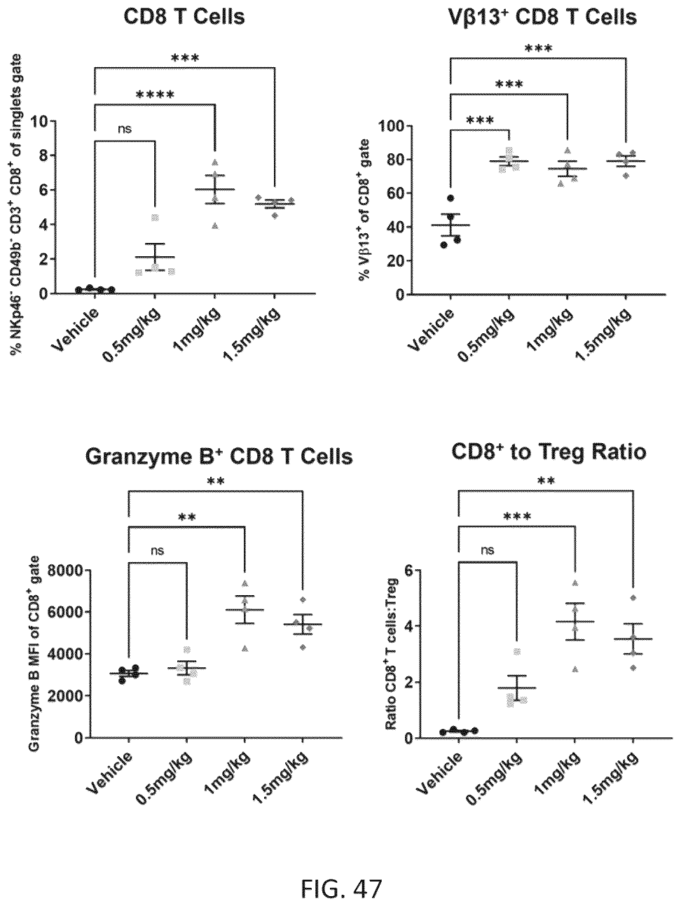

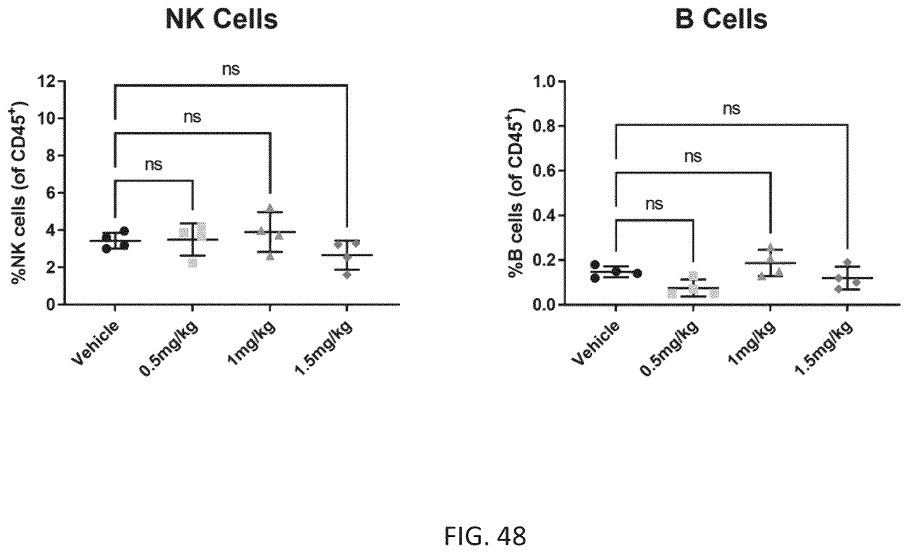

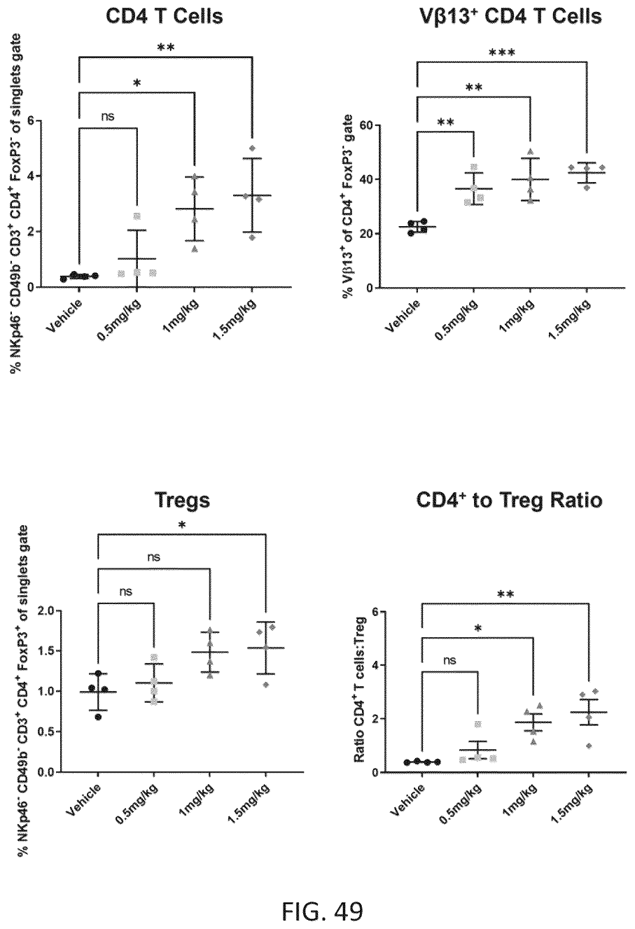

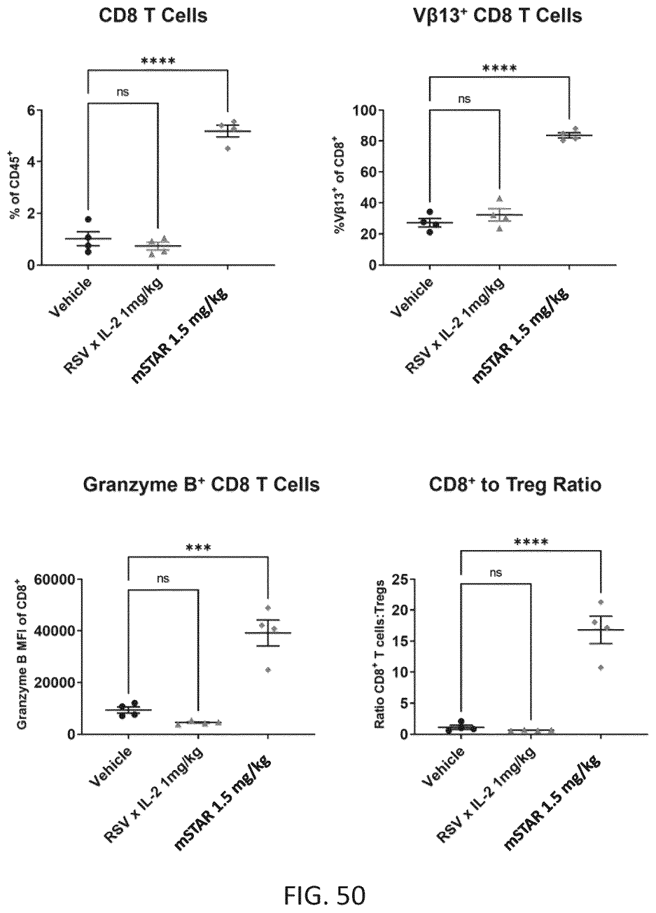

To further investigate the mechanism of action of mSTAR, more detailed assessments were performed in the EMT6 mouse model. In this model, mSTAR induced potent tumor regression compared with single arm control molecules, demonstrating the requirement for both anti-Vβ Fab and IL-2 arms of the molecule (FIG. 45). Compared to mice receiving vehicle, mice receiving mSTAR showed a marked accumulation of both CD8+ T cells and Granzyme B+ T cells as determined by immunohistochemistry staining (FIG. 46). Flow cytometry phenotyping of isolated EMT6 TILs also highlighted mSTAR dose related increases in the frequencies of total CD8+ T cells, Vβ13 CD8+ T cells, and Granzyme B+CD8+ T cells, in addition to a significant increase in the CD8:Treg ratio (FIG. 47). There were no changes in NK or B cell levels (FIG. 48). At doses of 1 mg/kg, mSTAR also significantly increased total CD4+ T cells and Vβ13 CD4+ T cells, without significantly altering Tregs (FIG. 49). Furthermore, TILs isolated from IL-2 control molecule treated mice at doses of 1 mg/kg did not increase total CD8+ T cells, Vβ13 CD8+ T cells, or Granzyme B+ CD8+ T cells (FIG. 50), in line with the lack of tumor growth inhibition with the IL-2 control molecule in the EMT6 breast carcinoma model.

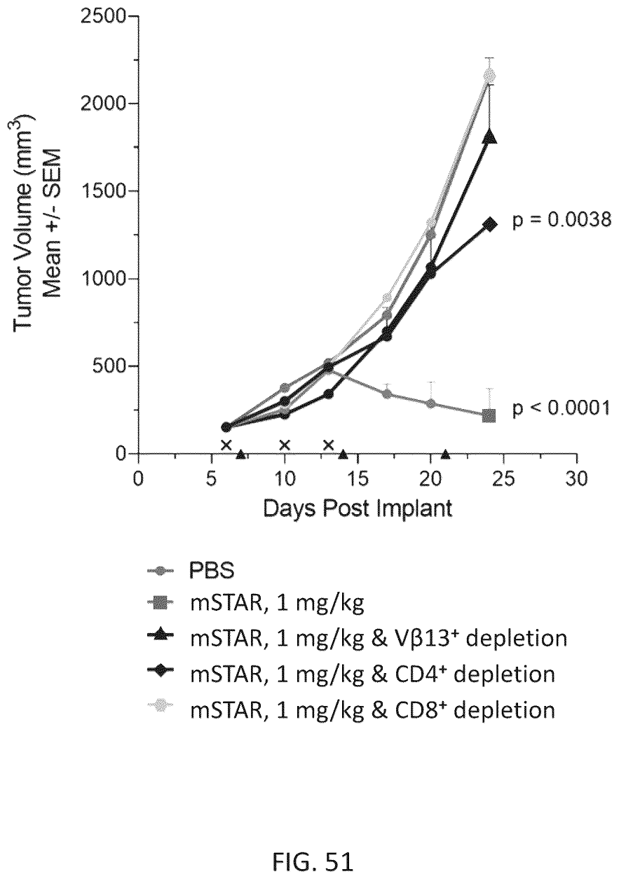

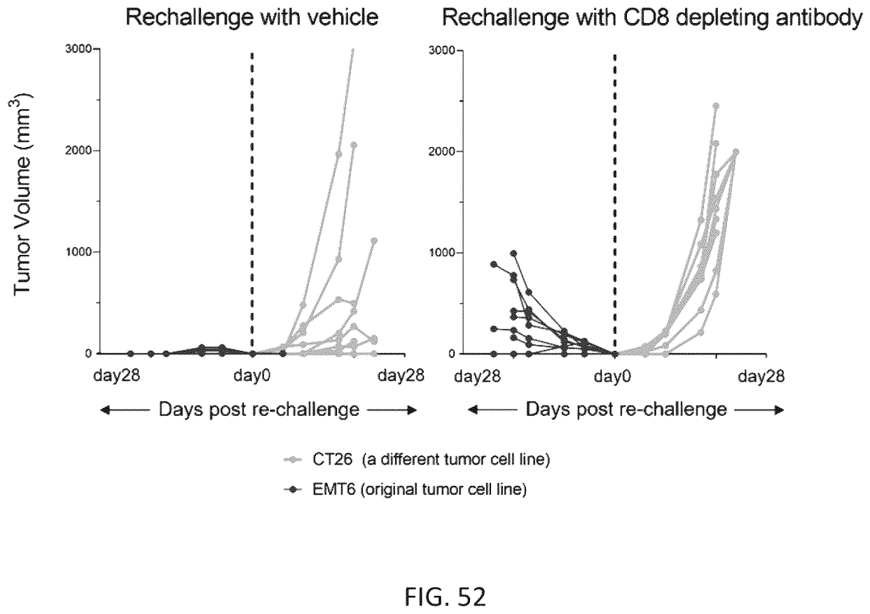

Selective depletion of Vβ13 T cells prior to treatment completely abrogated the anti-tumor activity of mSTAR in the EMT6 model (FIG. 51), suggesting the role of de novo expansion of Vβ13 T cells in the anti-tumor activity of mSTAR. Similarly, depletion of either CD8+ T cells or to a lesser extent CD4+ T cells also resulted in a loss of mSTAR anti-tumor activity. To investigate whether the accumulation of Vβ13 T cells in tumors also promoted long-term protective immunity, mSTAR-cured EMT6 mice were rechallenged 100-days post initial challenge. In all cured EMT6 mice, no tumor regrowth with EMT6 rechallenge was observed through 28 days without further mSTAR treatment (FIG. 52). In contrast, cured EMT6 mice did not reject a simultaneous de novo challenge with CT26 tumors in the opposite flank. These data showed that mSTAR promoted T cell-mediated immunity and long-term protection that is tumor specific.

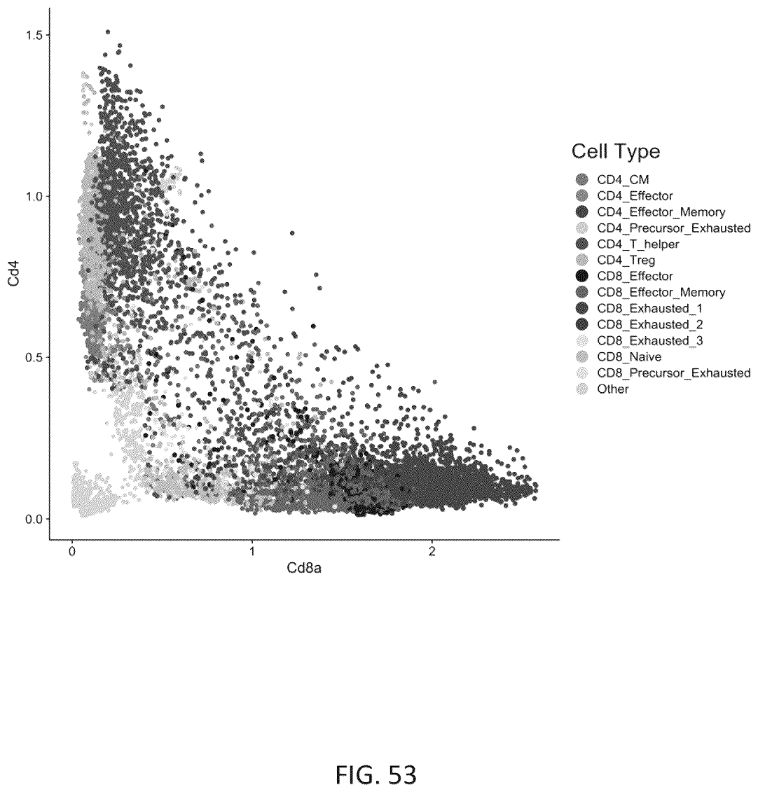

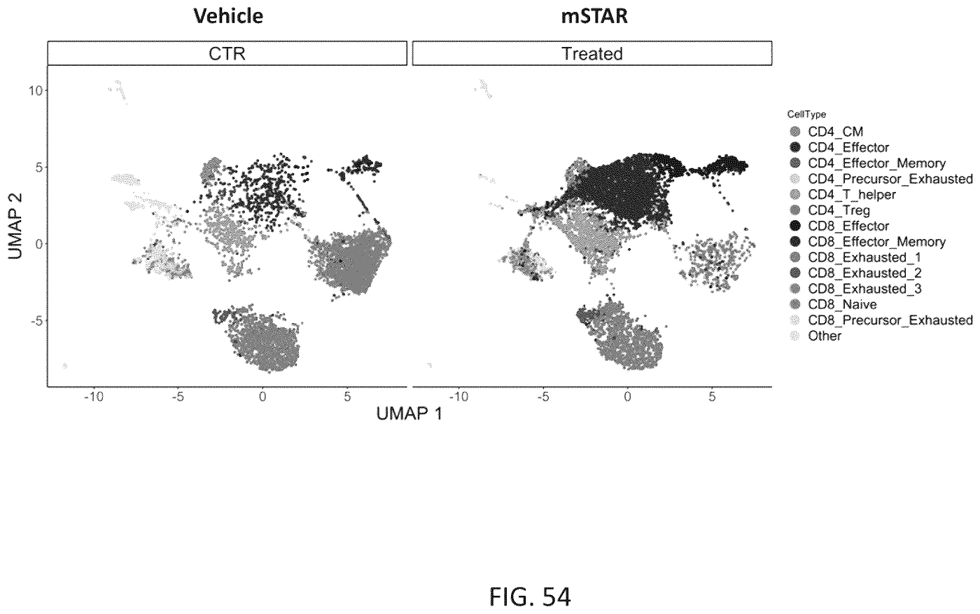

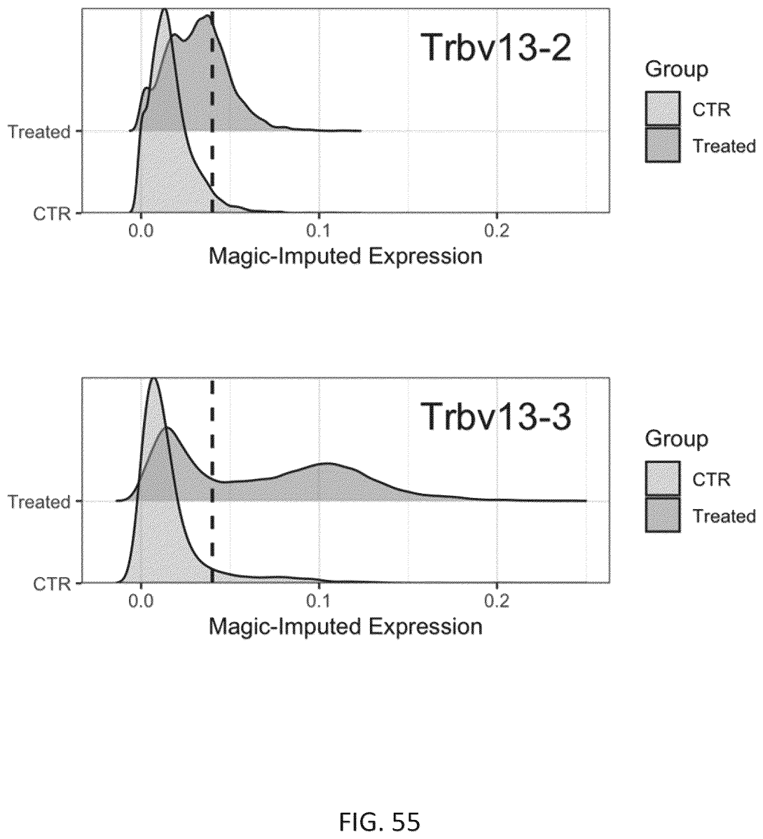

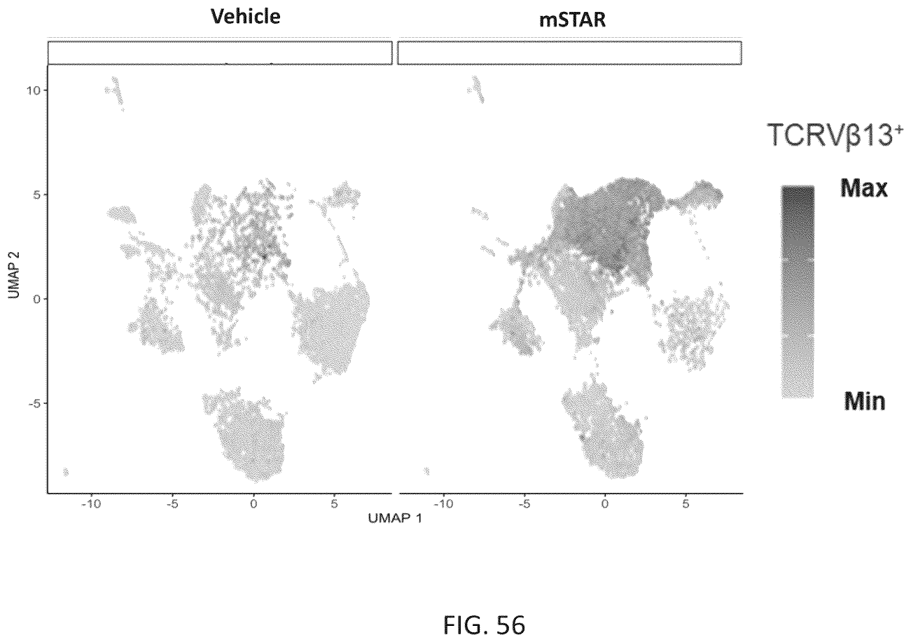

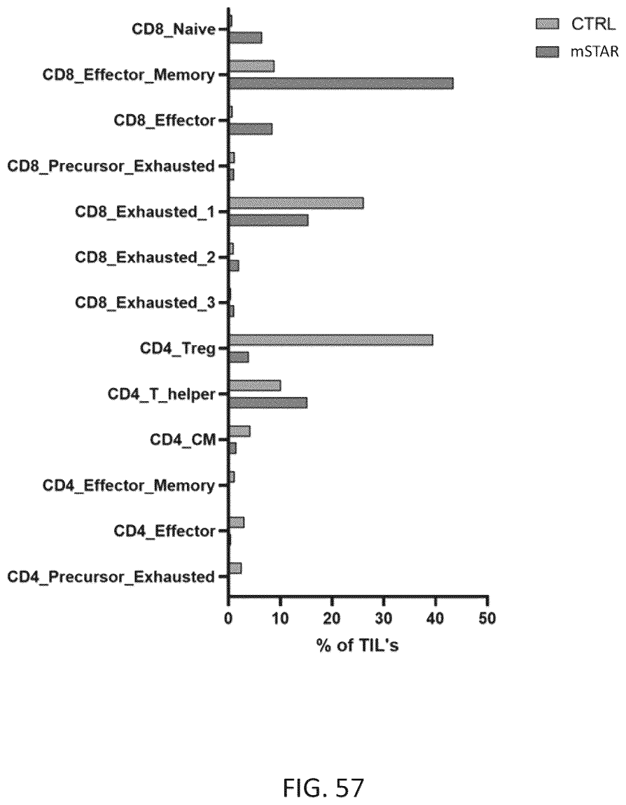

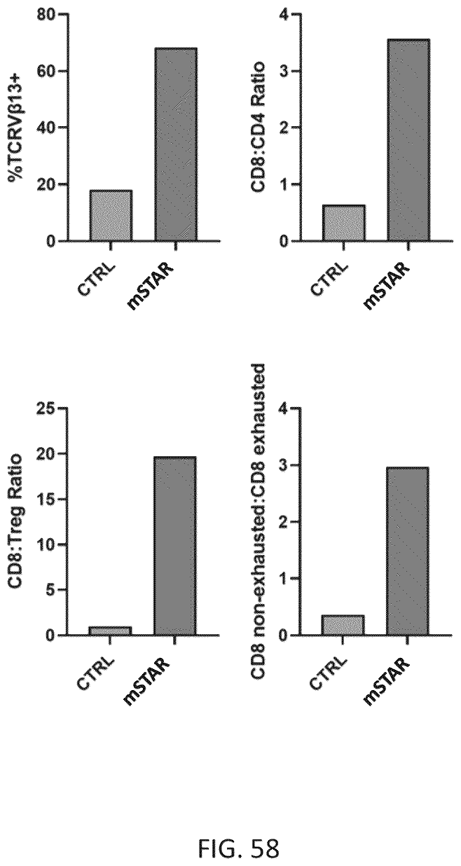

To better understand the immune mechanisms that promote tumor clearance by these Vβ13 T cells, a comprehensive, unbiased analysis of the EMT6 TIL transcriptome was performed at the singe cell level. CD4+ and CD8+ gene expression (FIG. 53) was weighted to assist for UMAP Seurat clustering and cell type annotations. As illustrated in the UMAP plot (FIG. 54), compared with TILs from vehicle treated EMT6 mice, mSTAR treatment profoundly remodeled the TIL compartment with increases in CD8+ Effector Memory T cells (T_{EM}), CD8+ Naïve T cells (T_{N}), and CD8+ Effector T cells (T_{EFF}), and CD4+ T helper cells. In contrast, significant reductions in Treg and CD8+ exhausted T cells were apparent following mSTAR treatment. Most Vβ13 T cells (inferred here through expression of TRBV13-2 and TRBV 13-2 transcripts (FIG. 55), mapped to the CD8+ T_{EM}, cluster (FIG. 56), and quantification of cell numbers further highlighted the remodeling of TILs in mSTAR-treated mice (FIG. 57). Consistent with flow cytometry data, scRNAseq data showed increased CD8:Treg ratios. CD8:CD4+ T cell ratios, and non-exhausted to exhausted CD8+ T cell ratios in mice treated with mSTAR compared to vehicle (FIG. 58).

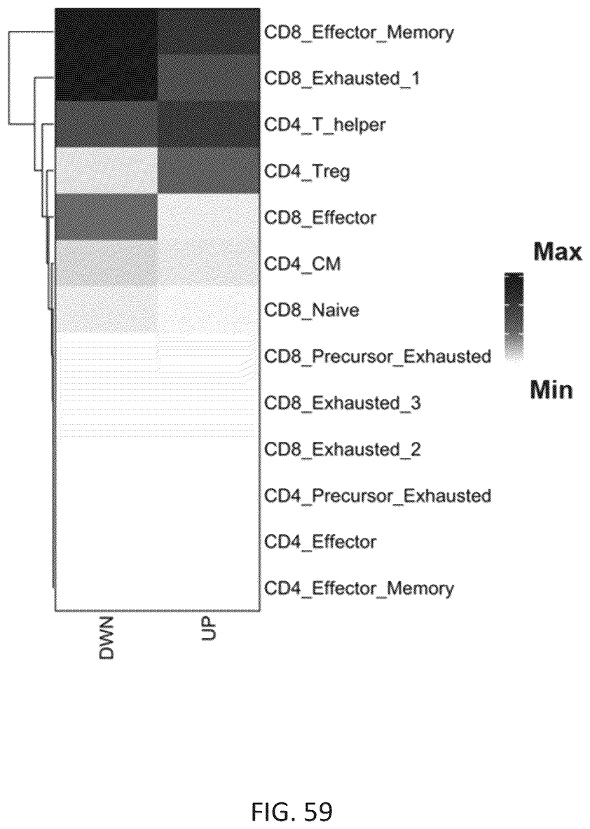

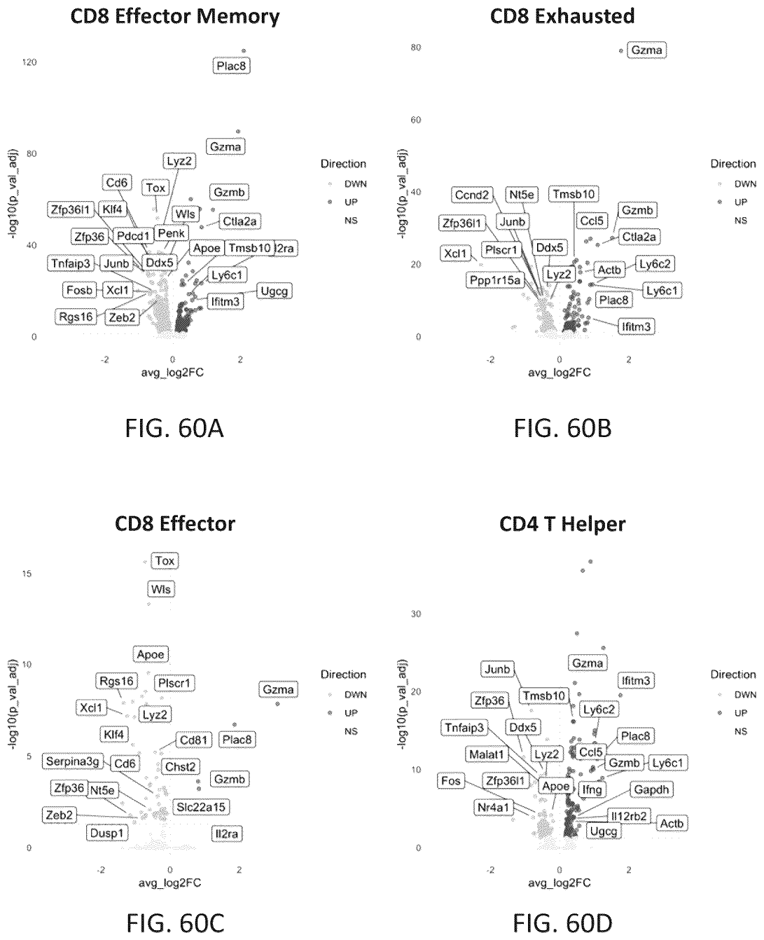

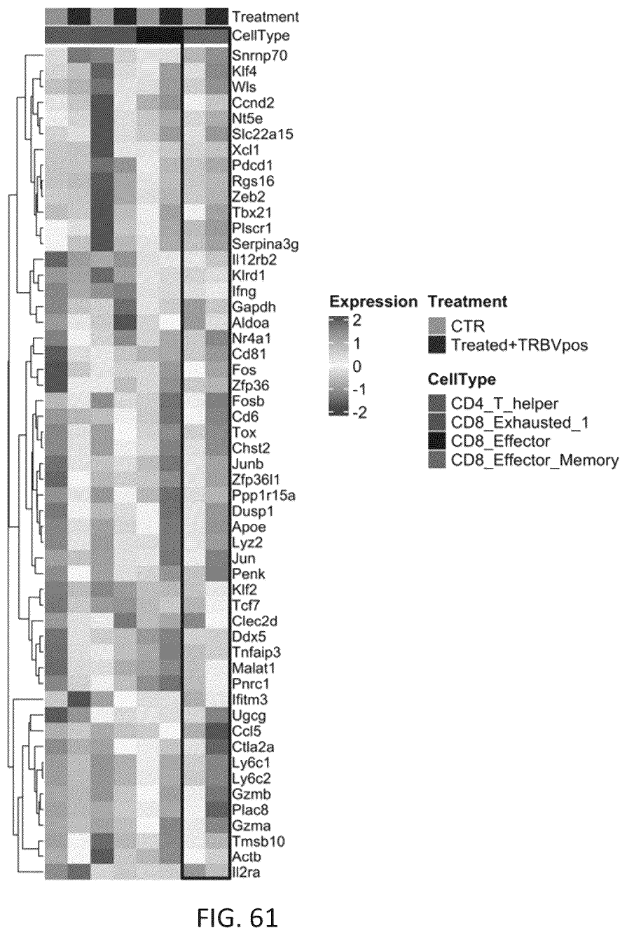

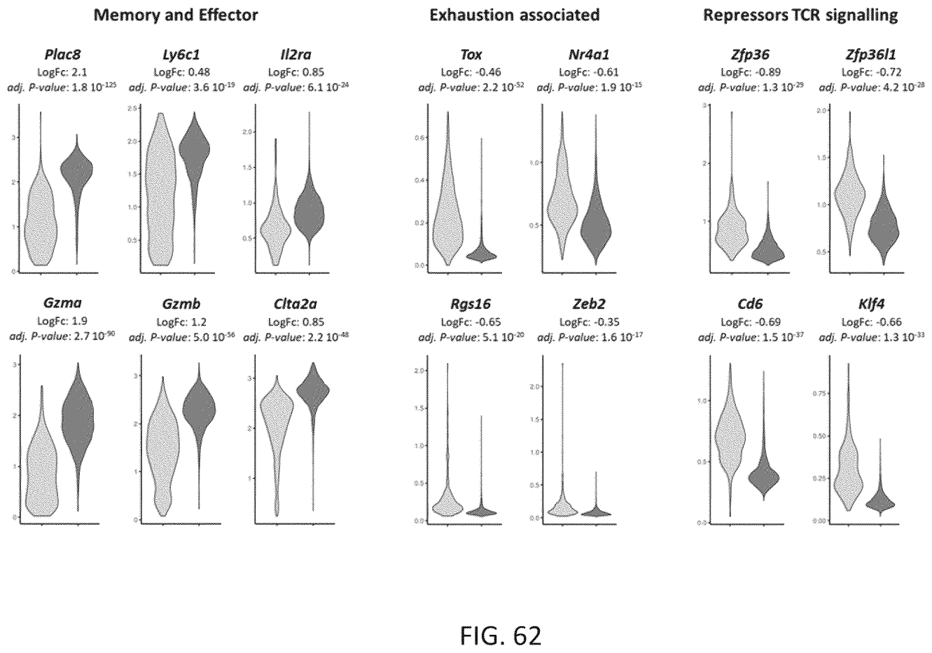

By comparing Vβ13 T cell subsets from mSTAR-treated versus vehicle-treated mice, the largest number of differentially expressed genes (DEGs) in CD8 TEM cells was identified followed by CD8 Exhausted_1, CD4+ T helper, Treg, and CD8+ TEFF clusters (FIG. 59). Volcano plots show individual DEGs from these Vβ13 T cells with recurring genes labeled (FIG. 60A-D). Analysis of genes consistently differentially expressed across Vβ13 CD4+ and CD8+ T cell subsets identified a novel gene signature (excluding ribosomal genes) specific for mSTAR treatment as illustrated in the heatmap shown in FIG. 61. This novel gene signature, most pronounced in the CD8+ T_{EM}, cells, was characterized by genes belonging to three distinct functional groups, including (a) an upregulation of T cell effector, memory, and cytotoxic genes, principally Plac8, Il2ra, Ctla2a, Ly6c1/2, Gzma, and Gzmb, (b) downregulation of genes associated with T cell exhaustion, such as Tox, Nr4a1, Zeb2 and Rgs16 (21-23), and (c) downregulation of repressors of TCR signaling, including Zfp36, Zfp6l1 (24). Cd6, and Klf4 (25, 26) (FIG. 62). The expression of several checkpoint genes such as Pdcd1 were also downregulated in Vβ13 TILs following mSTAR treatment. Taken together, this transcriptomic data showed that mSTAR treatment induced a shift in gene expression in Vβ13 TILs that promoted the acquisition of highly active TEM and TEFF cell phenotypes that could be protected from exhaustion and sensitized for TCR signaling.

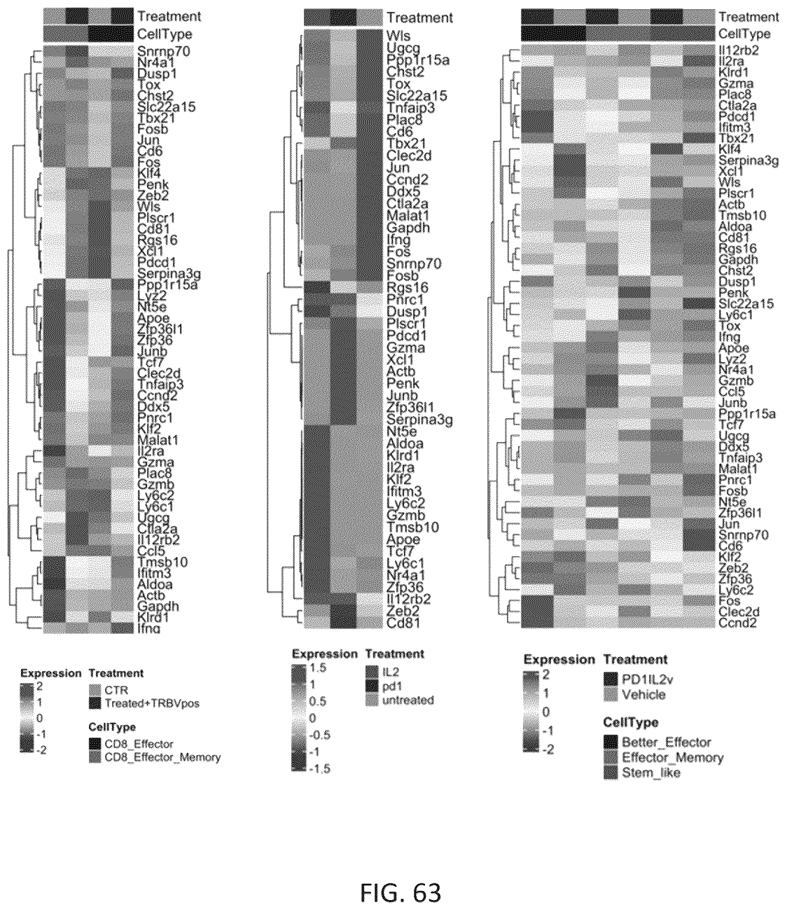

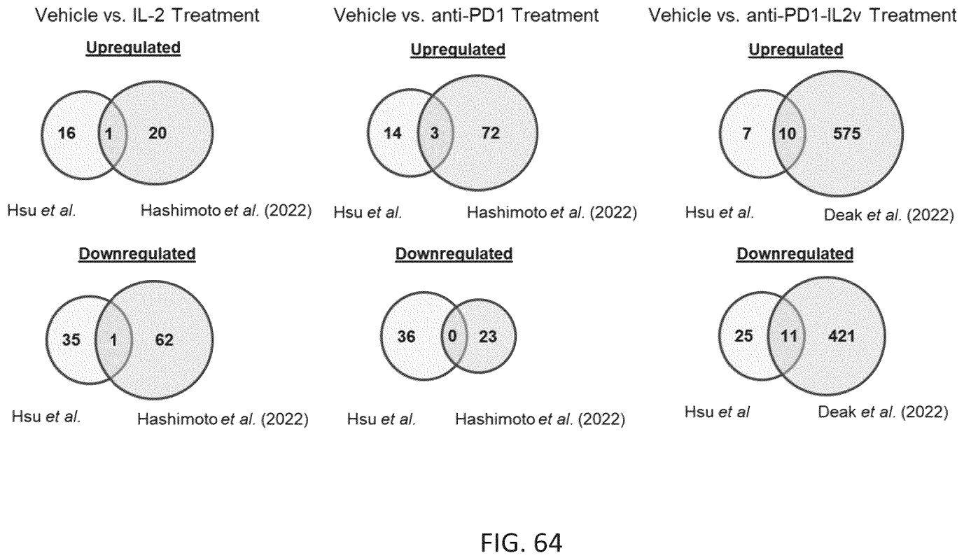

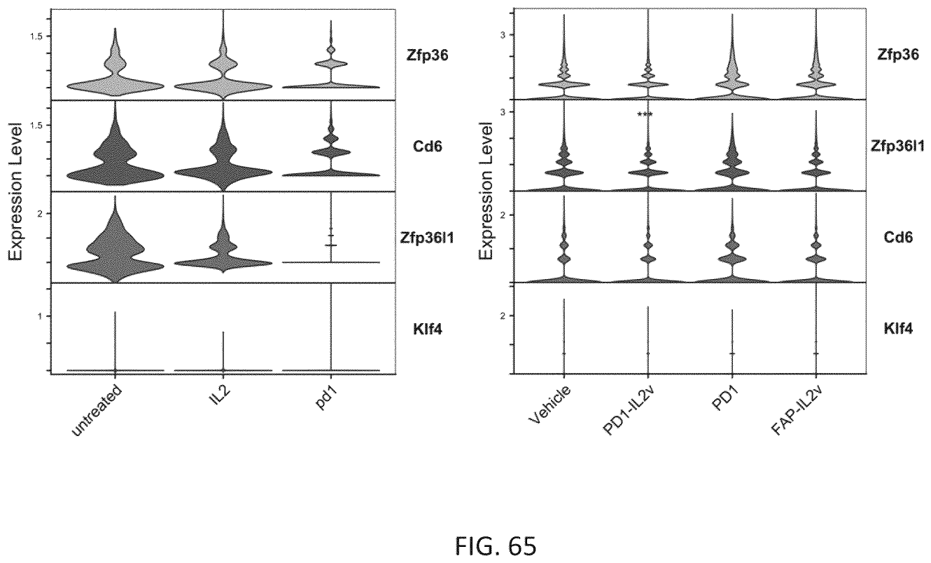

To investigate the specificity of the 53 gene signature identified in TILs from mice treated with mSTAR, genes signatures were compared with published scRNAseq data for IL-2, anti-PD-1, and an anti-PD-1-IL2 mutein (-IL2v) treatment in comparable murine syngeneic models and in similar CD8+ T cell subsets. Heatmaps for this 53-gene signature across these studies illustrated the distinct nature of the mSTAR induced gene expression (FIG. 63). In a gene overlap analysis restricted to those genes that were significantly and differentially expressed, only two of the 53 gene signatures were regulated in the same direction with IL-2 and three genes with anti-PD-1 treatment. The largest overlap of 21 genes shared out of 53 was observed when compared to genes regulated in CD8 ‘better effector’ T cells in tumor-bearing mice treated with an anti-PD-1-IL-2v bifunctional molecule (FIG. 64). Although a very small decrease for one of the TCR repressor genes (Zfp3611) was seen in TILs isolated from tumor-bearing mice treated with the PD-1-IL-2v molecule, in general the magnitude of decreases in TCR signaling repressor genes following mSTAR were not observed in the published data sets (FIG. 65), mSTAR treatment can induce a distinct shift in gene expression in Vβ13 TILs that promotes the acquisition of highly active T_{EM} and T_{EFF} cell phenotypes that can be protected from exhaustion and sensitized for TCR signaling.

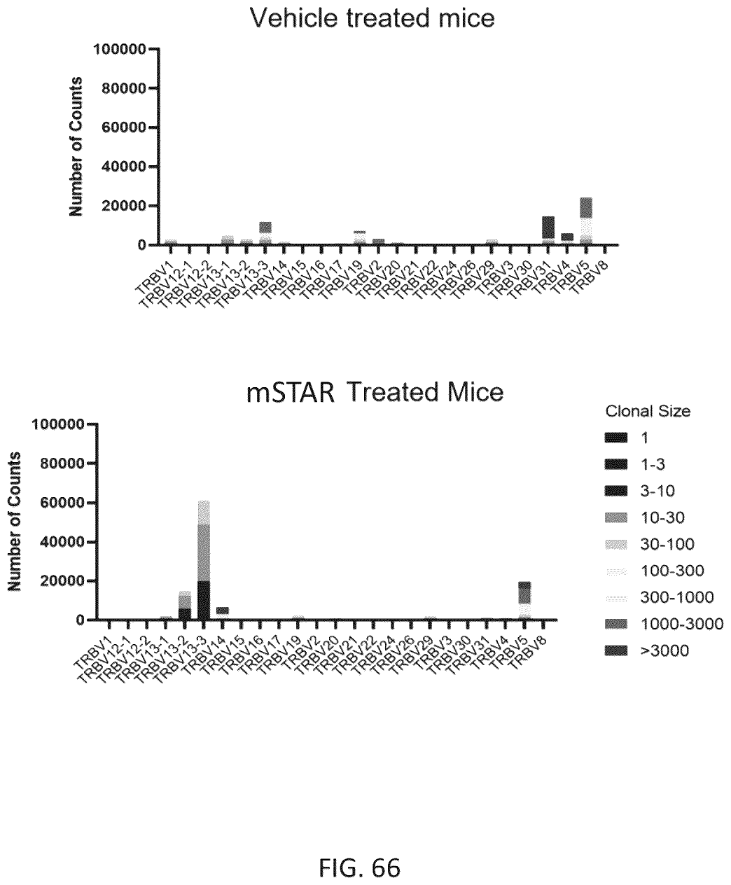

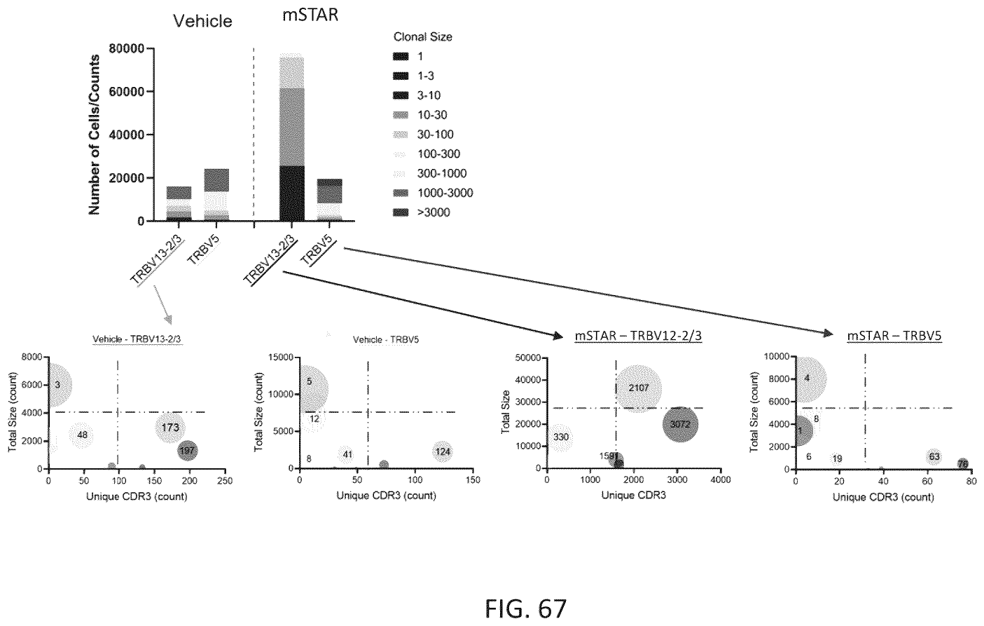

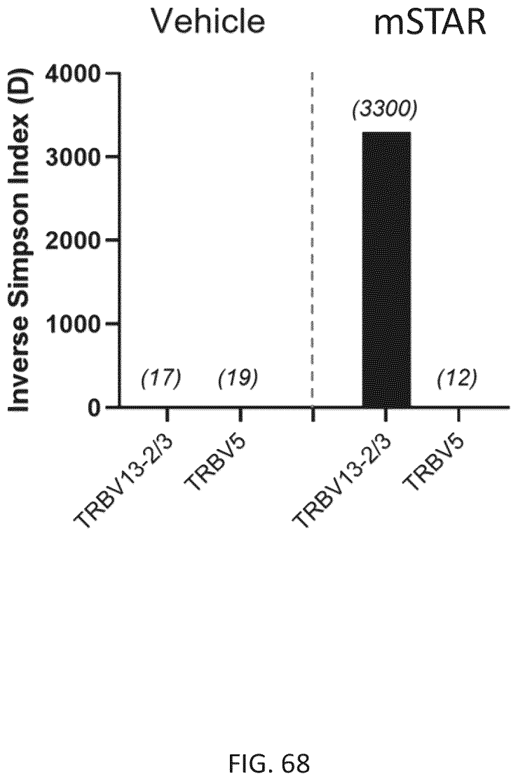

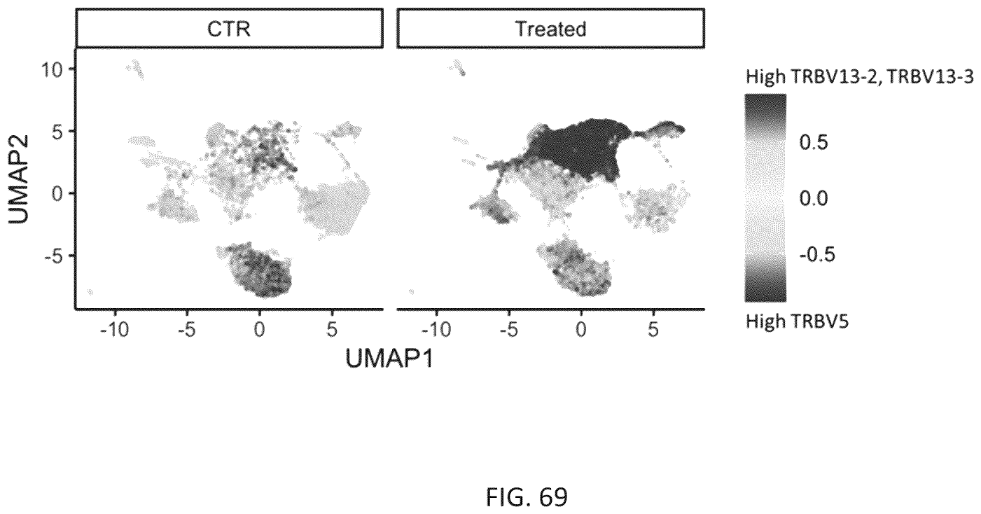

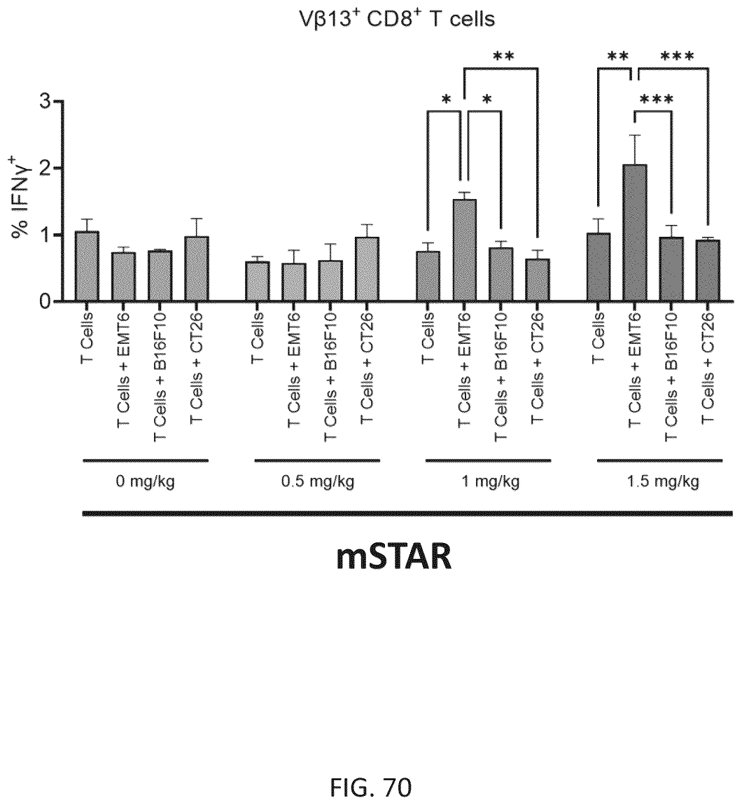

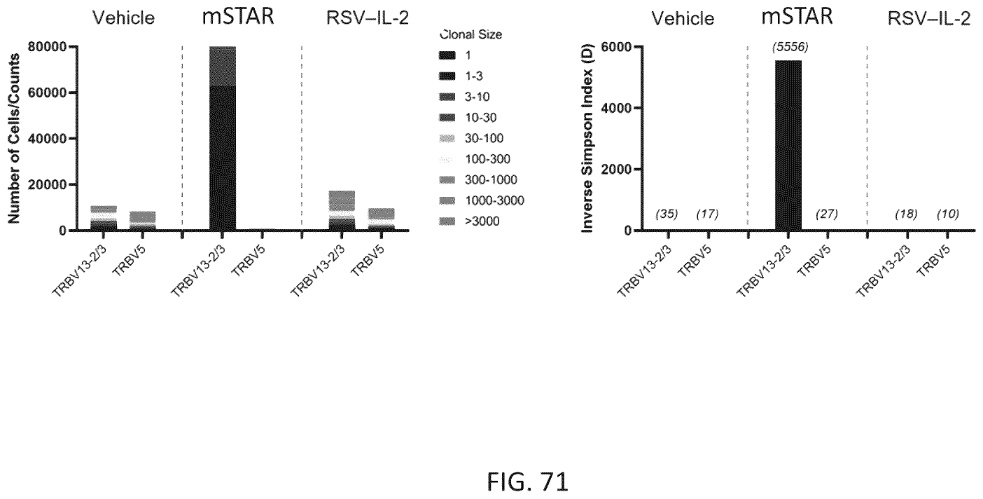

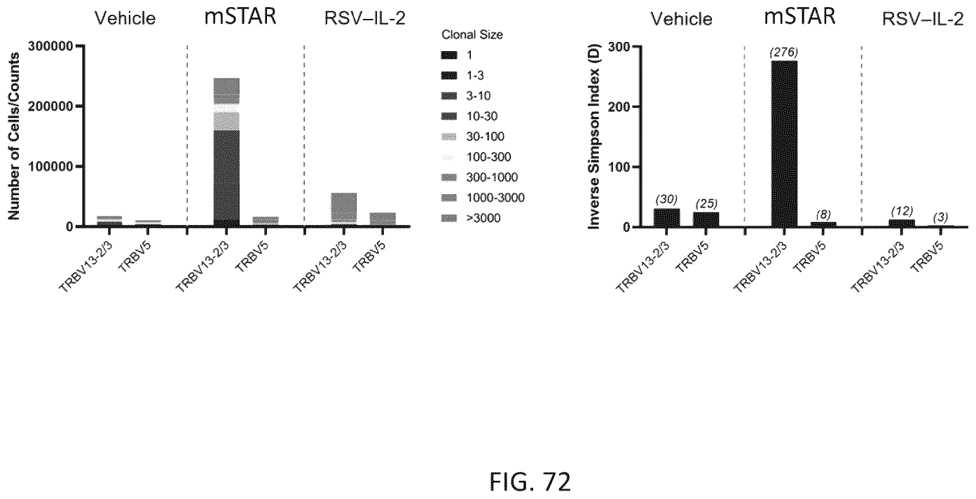

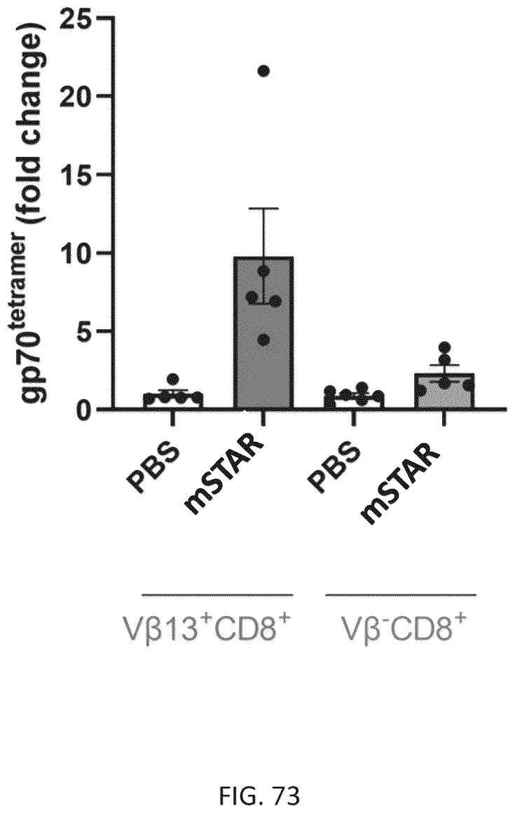

To further understand the impact of mSTAR treatment on TCR repertoire usage in TILs, TCR sequencing of treated EMT6 TILs was performed. TILs isolated from vehicle-treated EMT-6 mice generally exhibited oligoclonal TCRs with a few dominant T cell clones sharing the same CDR3 sequence and most clones expressed within the Vβ5 and Vβ13 subsets (FIG. 66). In contrast, TILs isolated from mSTAR-treated mice showed a striking increase in the number of unique CDR3s with smaller clonal sizes (indicating increased diversity of CDR3 repertoires) in the expanded Vβ13 TIL population. In contrast, the non-Vβ13 T cells (e.g., Vβ5) that were not targeted by mSTAR maintained a low TCR diversity with substantial single CDR3 clonal expansion. This increase in diversity was also apparent when plotting total CDR3 size vs unique CDR3 (FIG. 67) where counts are shown in a “bubble” plot for both Vβ13 and Vβ5 TILs from mice treated with mSTAR or vehicle. TCR diversity was also quantified using an inverse Simpson index that further reflects the increased repertoire diversity associated with mSTAR (FIG. 68). Of note, while most Vβ13 T cells expanded by mSTAR were T_{EM} cells, non-targeted Vβ5 cells were predominantly restricted to the CD8 exhausted cluster (FIG. 69). To further investigate the functional relevance of the observed increase in TIL CDR3 TCR repertoire diversity, an ex vivo antigen recall assay was performed on T cells isolated from spleens of EMT6 tumor-bearing mice treated with different doses of mSTAR. Using naïve splenocytes as antigen-presenting cells, splenocytes from mSTAR-treated EMT6 mice were cocultured with EMT6 tumor cell lysates (B16F10, and CT26 tumor cell lysates were used as negative controls) to explore the tumor specificity of T cell responses using intracellular IFN-γ staining. Using this ex vivo recall assay, significant T cell responses were observed in Vβ13 CD8+ T cells from mSTAR-treated animals challenged with EMT6 tumor cell lysates, whereas no T cell activation was observed in response to challenge with B16F10 or CT26 tumor cell lysates (FIG. 70). Consistent with the data from EMT6 TILs. TCR diversity as determined by clonal sizes and the inverse Simpson index was also increased substantially in Vβ13 T cells after STAR1302 treatment of mice bearing MC38 tumors (FIG. 71) or CT26 tumor (FIG. 72). This increase was not observed for mice treated with the single arm IL-2 control. Finally, to further confirm the repertoire of tumor antigen-specific T cell responses within Vβ13 T cells, TILs isolated from mSTAR-treated CT26 tumors were stained with tetramers to recognize the tumor-rejection antigen AH1/gp70 (a murine leukemia virus (MuLV) envelope protein). Significantly higher levels of AH1/gp70+CD8+ T cells were present in Vβ13 CD8+ T cells from mSTAR-treated mice compared to PBS-treated mice, with minimal levels of AH1/gp70-specific specific T cells present in non-Vβ13 T cells (FIG. 73). By matching antigen-specific TCR CDR3 sequences from AH1/gp70-specific murine T cells to CDR3 sequences within the Vβ13 (TRBV13) repertoire from mSTAR-treated mice, approximately 20 known AH1/gp70 CDR3 sequences were identified that were not observed in Vβ13T cells from PBS-treated mice (Table SS).

TABLE SS
AH1-specific CDR3 sequences found only in
Vβ13+ TILs from each mSTAR treated mice
bearing CT26 tumors

| SEQ | |
|---|---|
| ID | |
| NO: | |
| | mSTAR |
| 4033 | CASGDAWGSAETLYF |
| 4034 | CASGDGWGGAETLYF |
| 4035 | CASGDLGYEQYF |
| 4036 | CASGTGAYEQYF |
| 4037 | CASSRQNSDYTF |
| 4038 | CASSAGTTEVFF |
| 4039 | CASAGGYEQYF |
| 4040 | CASRTGGSYEQYF |
| 4041 | CASSAGGSDYTF |
| 4042 | CASSDAGYEQYF |
| 4043 | CASSDAQYEQYF |
| 4044 | CASSDGGSYEQYF |
| 4045 | CASSDGRYEQYF |
| 4046 | CASSDGSYEQYF |
| 4047 | CASSDGTGYEQYF |
| 4048 | CASSDNSGNTLYF |
| 4049 | CASSEGSYEQYF |
| 4050 | CASSGDWGDEQYF |
| 4051 | CASSLTGGAETLYF |
| 4052 | CASSPGSYEQYF |
| | PBS (Control) |
| 4053 | CASQDAWGVAETLYF |
| 4054 | CASSAFYEQYF |
| 4055 | CASSDGWGYEQYF |

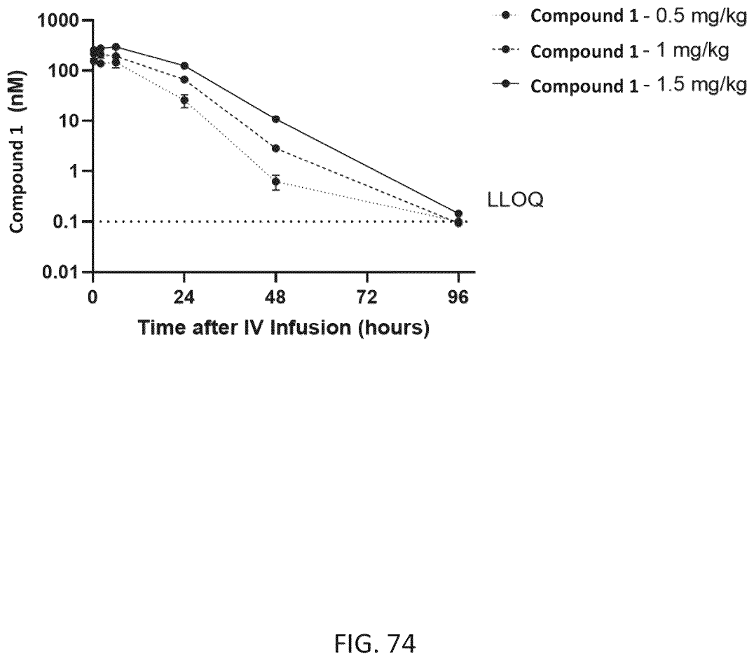

To support the characterization of Compound 1 in non-human primates, cross-reactivity was confirmed by measuring the binding of Compound 1 to purified cynomolgus Vβ6 TCR and IL2R proteins. Compound 1 bound to cynomolgus Vβ6-2 (4.9 nM), IL2Rα (CD25, 48 nM), and IL2Rβγ (3.3 nM) with high affinity that was similar to binding with human Vβ6 TCR and IL-2R proteins (Table S1). The pharmacokinetics of a single intravenous (IV) dose of Compound 1 was characterized by rapid non-linear clearance over the dose range tested (FIG. 74). Further details of Compound 1 pharmacokinetics are shown in Table S6.

TABLE S6
Pharmacokinetic parameters of Compound 1 in cynomolgus monkeys.

| | Unit | 0.5 mg/kg | 1 mg/kg | 1.5 mg/kg |
|---|---|---|---|---|
| T ½ | h | 5.3 | 8.1 | 11.0 |
| Tmax | h | 0.25 | 0.25 | 6 |
| Cmax | nM | 154.3 | 217.1 | 297.4 |
| AUC 0-t | nM*h | 2712.0 | 4448.0 | 7358.4 |

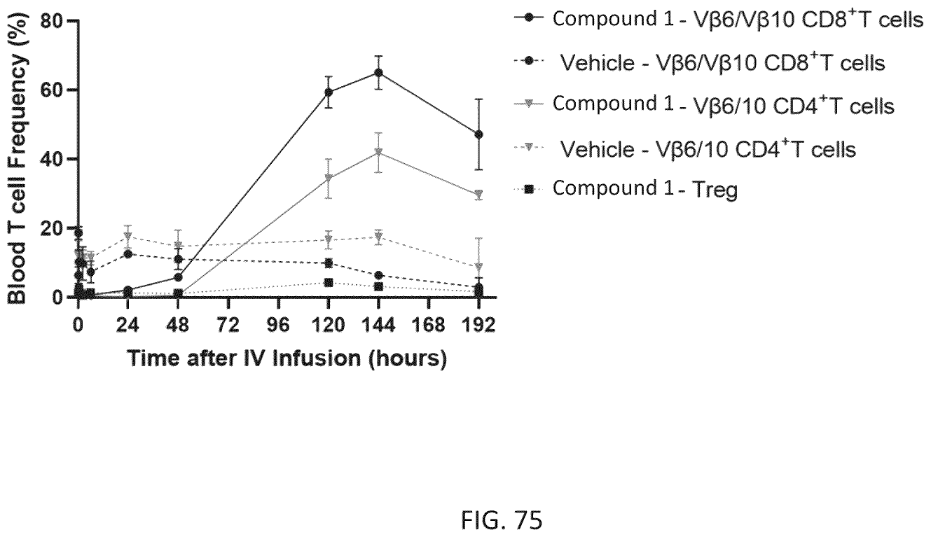

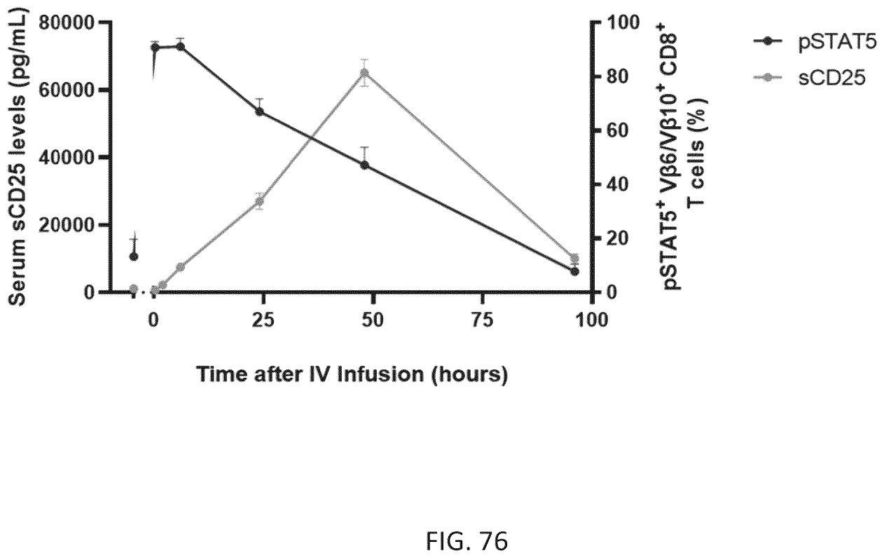

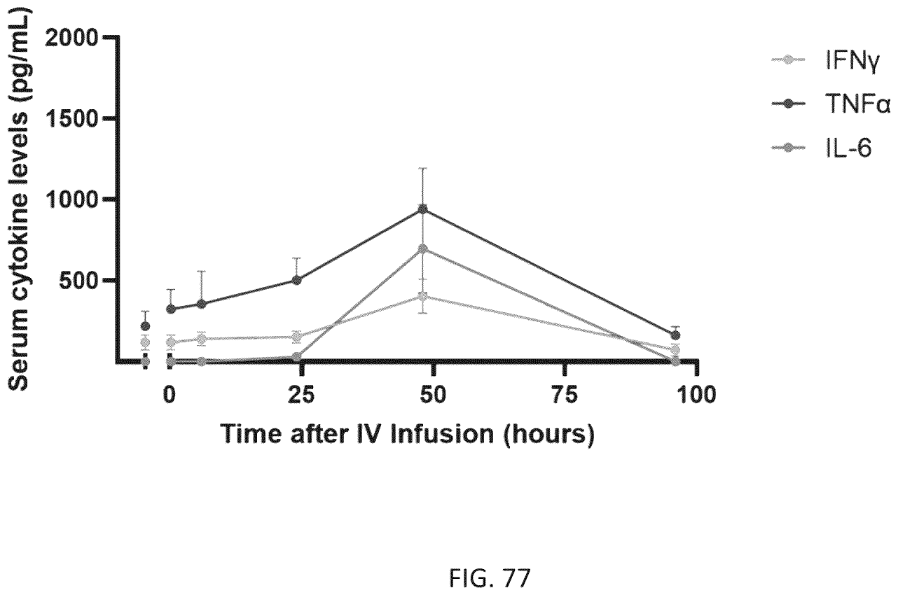

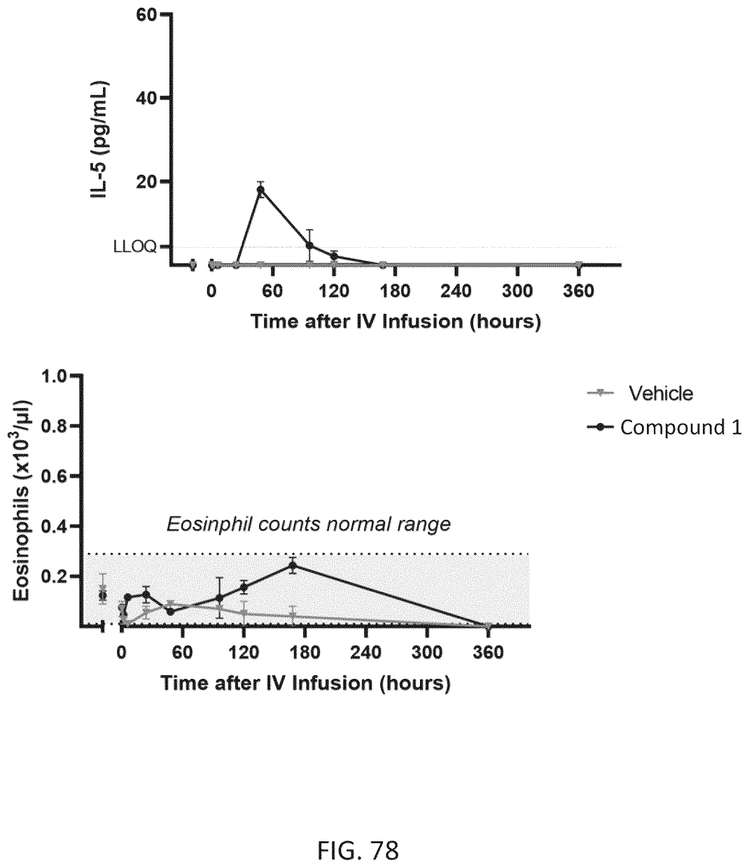

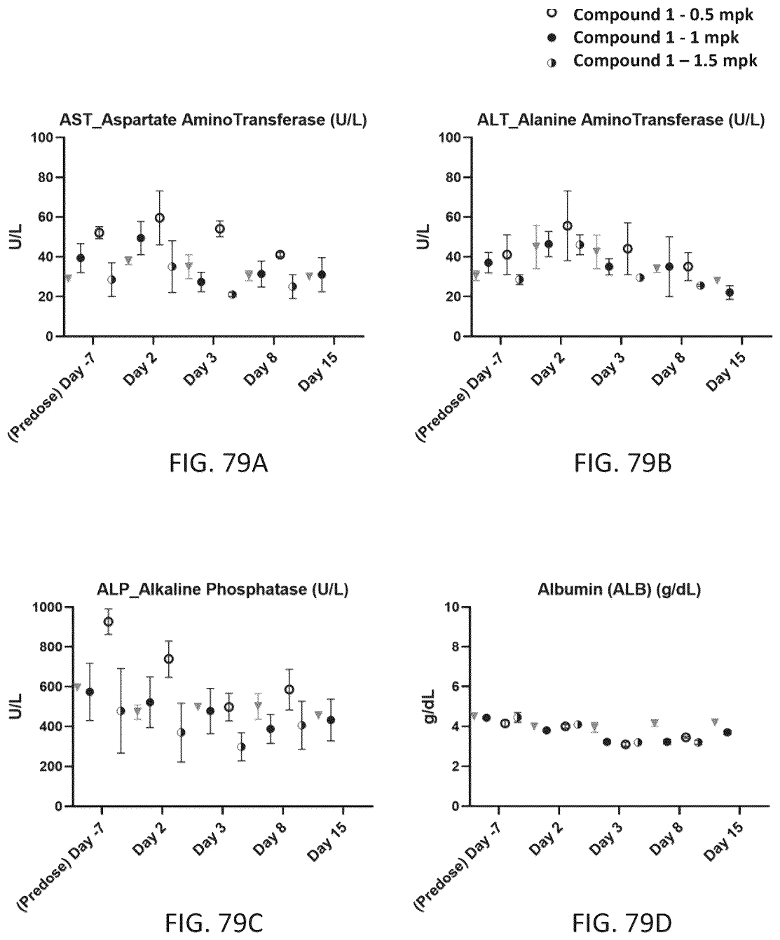

Despite this rapid clearance, sustained expansion of target Vβ6/Vβ10 CD8+ and, to a lesser extent, CD4+ T cells (2-3-fold increase over baseline) was observed in the blood of Compound 1-treated monkeys, peaking at day 6 post infusion in the absence of significant expansion of FoxP3+ Treg cells (FIG. 75). This V 6N10 lymphocytosis was more profound in the CD8+ T cell compartment and was preceded by transient peripheral lymphopenia due to tissue margination of activated T cells immediately after dosing. The magnitude of Vβ6/Vβ10 T cell expansion observed in the blood of monkeys was consistent with Vβ13 expansion associated with antitumor activity of mSTAR in tumor-bearing mice. To confirm engagement of the IL-2R pathway by Compound 1, pSTAT5 and soluble CD25 were assessed in PBMCs and serum from dosed monkeys. pSTAT5 levels increased immediately following dosing with Compound 1 indicating significant activation of the IL-2R pathway, with sCD25 levels peaking at 48 hours post infusion (FIG. 76). Finally, only moderate cytokine release was observed in the blood of monkeys following infusion of Compound 1 (FIG. 77). Cytokine levels generally peaked around 48 hours following Compound 1 and peaked at 0.4 ng/ml for IFN-γ, 0.7 ng/ml for IL-6 and 1.0 ng/ml for TNFα. These peak levels were generally delayed by 42 hrs, albeit at relatively low levels (50-90% lower compared to other reports of cytokine release in monkeys with IV dosing of bispecific anti-CD3 antibodies). Regarding the potential for IL-2-related toxicities, a minimal increase in peripheral IL-5 levels and cosinophils counts was observed (established markers of IL-2 toxicity in monkeys dosed with 1 mg/kg Compound 1 (FIG. 78). Similarly, no discernible changes in serum concentrations of liver enzyme markers of IL-2-induced hepatic injury were observed across 0.5-1.5 mg/kg Compound 1 doses (FIGS. 79A-D). Furthermore, no serious toxicities, body weight loss, or deaths were observed following dosing of IV Compound 1 in cynomolgus monkeys.

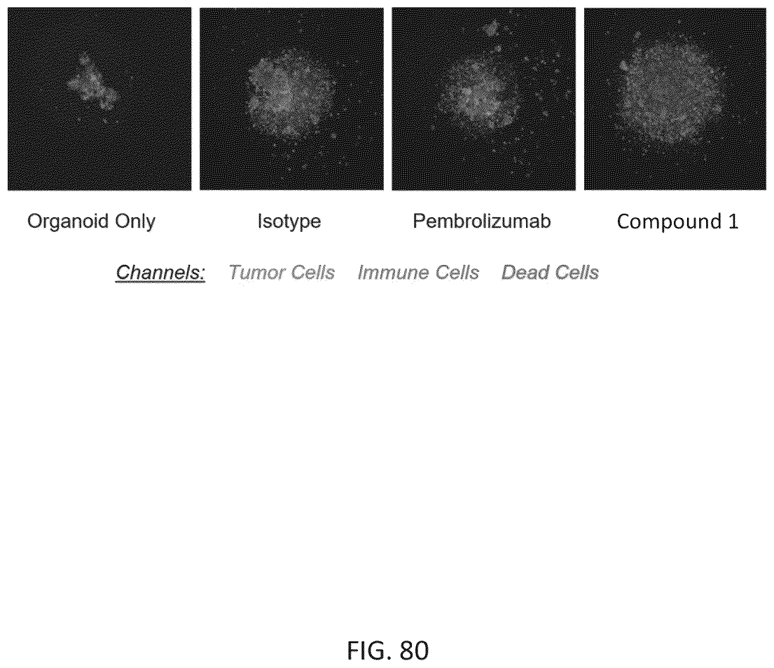

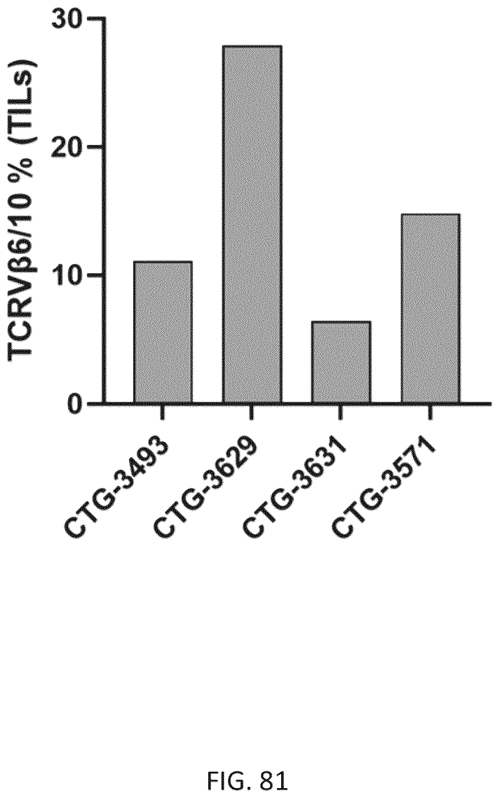

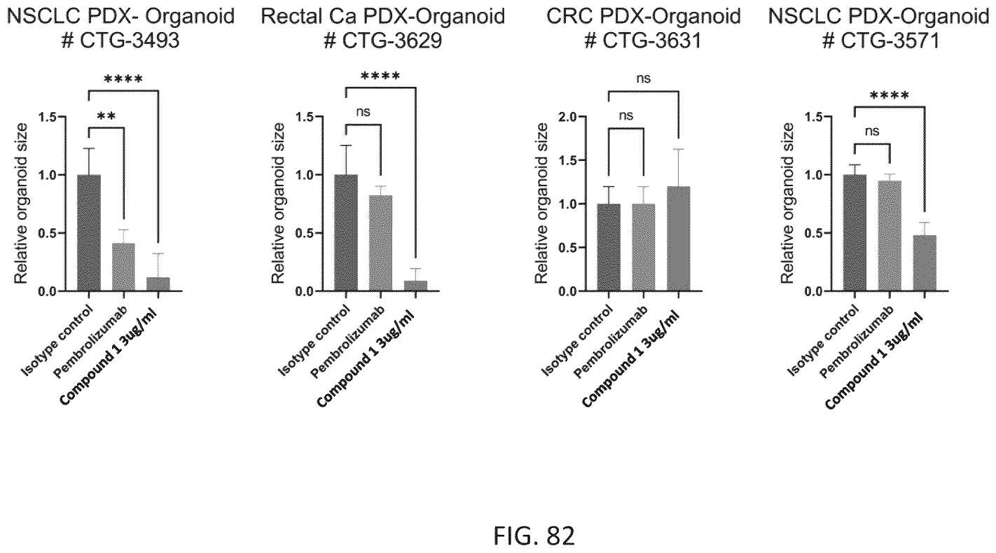

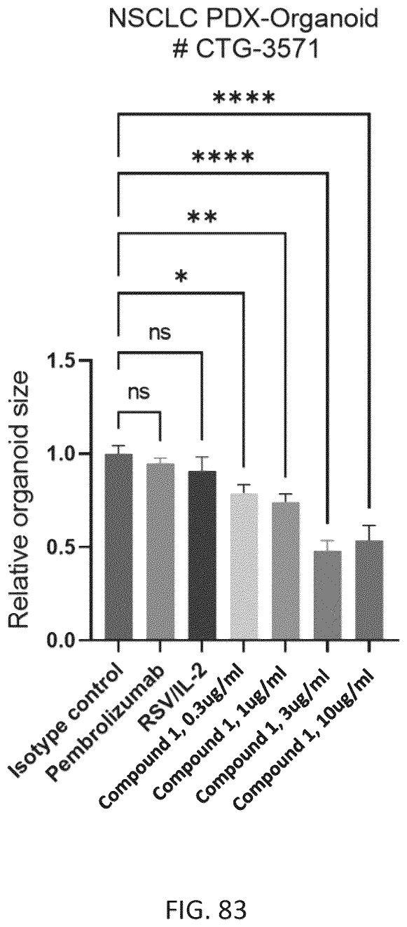

As shown in representative images from an ex vivo human tumor organoid culture derived from a rectal cancer patient. Compound 1 promoted the expansion of human TILs, effectively reduced organoid size, and increased the proportion of dead cells (FIG. 80). The frequency of Vβ6/Vβ10 T cells varied among the 4 organoid models tested, ranging from 6 to 28% of autologous TILs (FIG. 81), with three of these models being refractory to pembrolizumab. In these studies, Compound 1 effectively reduced organoid size in 3 of 4 models, two of which were refractory to pembrolizumab (FIG. 82). Furthermore, reduction of organoid size was dependent on Compound 1 concentration and was not observed for the IL-2 control molecule (FIG. 83).

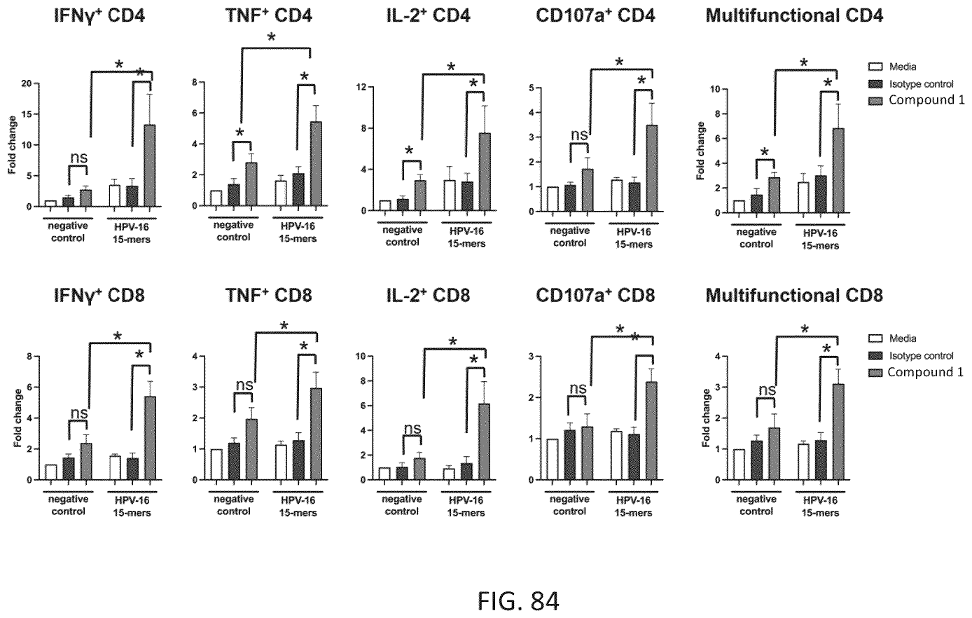

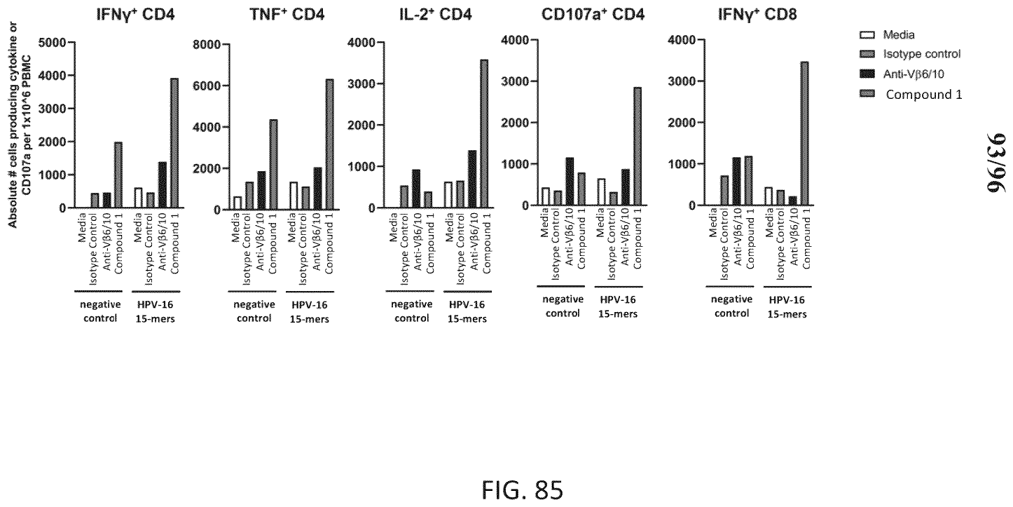

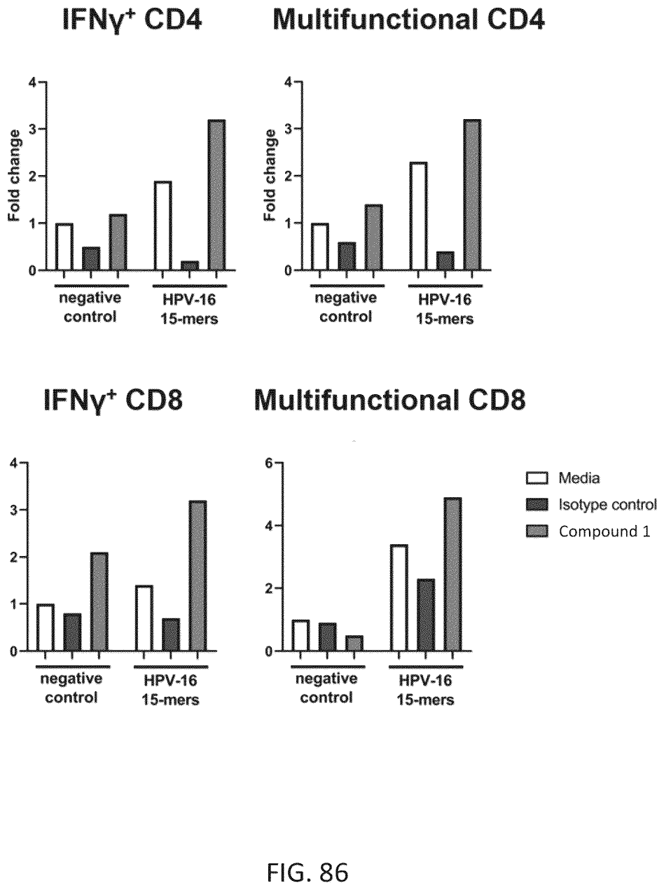

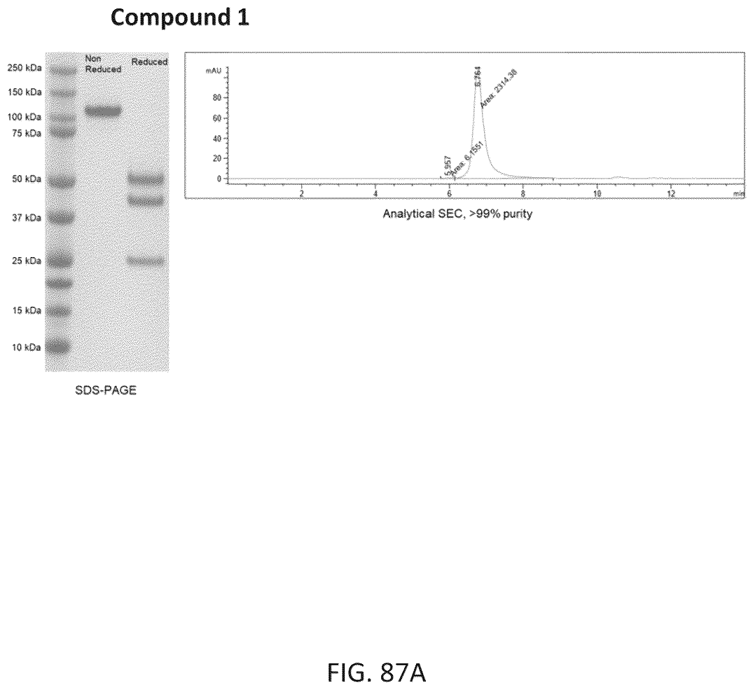

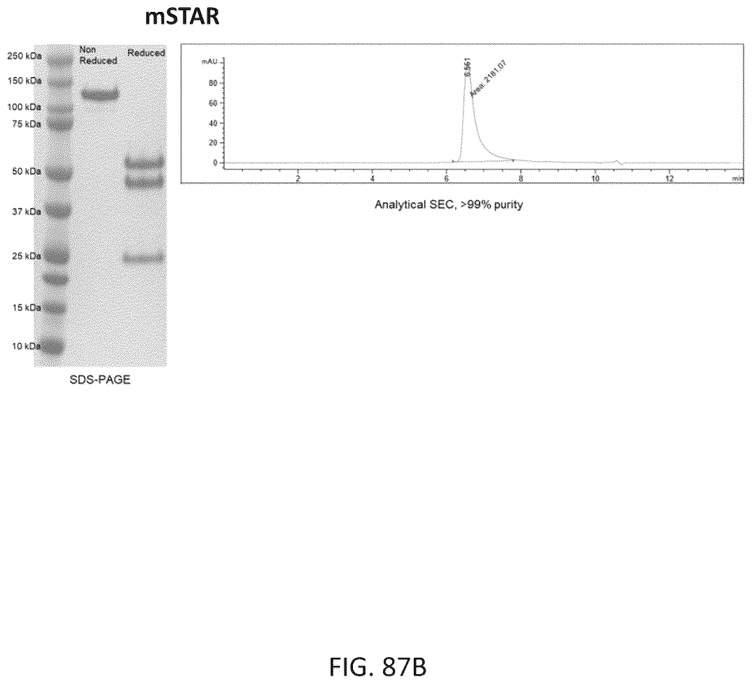

Finally, the ability of Compound 1 to expand human antigen specific T cells in vitro was investigated. As shown in FIG. 84, Compound 1 significantly expanded HPV-16 specific CD4+ and CD8+ T cells when stimulated with HPV-16 peptides, as evidenced by enhanced numbers of T cells producing IFN-γ, TNF-α, IL-2, and positive for CD107a, compared to the isotype control. This expansion was not observed with the soluble anti-Vβ6/Vβ10 antibody control (FIG. 85). Notably, Compound 1 expanded both multifunctional CD4+ and CD8+ HPV-16 specific T cells, defined as T cells positive for 2 or more of the functional markers examined. Similar findings were observed in PBMCs from a cervical cancer patient. Here, Compound 1 increased the number of HPV-16 CD4+ and CD8+ T cells producing IFNγ and multifunctional HPV-16 CD4+ and CD8+ T cells (FIG. 86).

### Discussion

To support the development of a pan-tumor T cell immunotherapy, in this Example, Compound 1 was designed to target Vβ6 T cell subsets, since Vβ6 T cells can be enriched in TILs relative to other Vβ subsets and are present in all cancers. By expanding this smaller subset of T cells over 5-7 days, rather than activating all T cells as with agonist anti-CD3 antibodies, it was hypothesized that adverse events associated with T cell activation (e.g., cytokine release) might be reduced.

These studies demonstrate that the Compound 1 molecule, comprising monovalent binding to Vβ6/Vβ10 TCRs and IL-2 binding to IL-2 receptors, selectively activates and expands human Vβ6/Vβ10 T cells wherein both arms are necessary for T cell activation. Prior studies of TCR activating antibodies suggest a requirement for TCR crosslinking with bivalent antibodies and/or presentation on a solid phase. However, it is shown here that in solution, a monovalent TCR binding Fab fused to a cytokine ligand could robustly stimulate both TCR and IL-2R signaling and recapitulated the T cell characteristics induced by plate coated anti-Vβ6 antibodies. TCR engagement by Compound 1 may involve clustering of IL-2Rs with Vβ6 TCRs on cellular lipid rafts, or a novel allosteric mode of TCR activation. Compound 1 also showed avidity-enhanced binding of the IL-2 moiety in cis in an anti-TCR Fab-dependent manner, thus limiting the potential for on-target/off-tissue toxicity associated with native IL-2 therapies. Indeed, neither histological evidence of vascular injury nor signs of liver inflammation were observed in mice dosed with mSTAR at high doses compared with rhIL-2, wherein signs of IL-2-mediated inflammatory toxicity were prevalent.

Consistent with studies of plate coated anti-Vβ6 antibodies, Compound 1 induced the selective expansion of both CD8+ and CD4+ human Vβ6/Vβ10 T cells that acquired a homogenous CD95+CCR7+CD45RA− central memory (Tc,) phenotype that was considered atypical due to the concomitant expression of cytotoxic and effector molecules such as IFN-γ, Granzyme B and CD25.

The significant tumor regression observed in mice treated with the murine surrogate molecule mSTAR compared with vehicle was durable with cures maintained through >100 days and long-term protection from tumor re-challenge that was dependent on CD8+ T cells. The infiltration of activated CD8+Granzyme B+Vβ13 T cells into the usually immune-excluded EMT6 tumors, and the loss of anti-tumor activity with depletion of these cells, strongly implicated the de novo expansion of memory Vβ13 T cells in the mechanism of action of mSTAR.

The analysis of Seurat clusters from EMT6 TILs further highlighted the mSTAR-induced expansion of Vβ13 T cells in murine tumors with profound remodeling toward a population of Vβ13 CD8+ TEM cells and a significant decrease in FoxP3+ Treg cells. Deeper analysis of the transcriptome of these expanded Vβ13 T cells revealed a gene signature characterized by upregulation of Plac8, with Ctla2a, Il2ra, and the T cell memory/effector gene Ly6c1 across numerous T cell subtypes. Without wishing to be bound by a certain theory, this suggested significant reprogramming to a mixed effector/memory phenotype reminiscent of the phenotype induced in human T cells by Compound 1.

Several important genes were downregulated in Vβ13 TEM cells, including immune checkpoints and the transcription factors Tox, Nr4al and Rgs16 that have been implicated in transcriptional control of exhausted T cells. Without wishing to be bound by a certain theory, reduced expression of several TCR regulatory genes including the RNA binding repressor proteins Zfp36 and Zfp36l1 and other TCR signaling repressors (e.g., Cd6 and Klf4) that control the tempo of T cell activation suggested that de-repression of TCR signaling by mSTAR might also be a mechanism through which treated TILs achieve higher effector potentials.

Without wishing to be bound by a certain theory, taken together with the increases in CDR3 repertoire diversity, these data suggest mSTAR anti-tumor activity is mediated by the significant de novo expansion of a clonally diverse population of Vβ13 CD8+ effector memory T cells and CD4+Th1 memory T cells. This apparent clonal “revival” within expanded Vβ13 TILs suggests expansion of low abundance tumor-resident T cells, or significant clonal replacement through expansion and infiltration of Vβ13 T cell precursors from other niches. Nevertheless, the clonal enrichment observed in TILs from mice treated with mSTAR may reflect the development of more productive T cell responses to low affinity tumor neoantigens, and/or the development of de novo T cell responses to hitherto untargeted tumor neoantigens. In contrast, response to anti-PD-1 therapy in humans is associated with limited expansion of mainly precursor exhausted T cells (T_{EMP}) with modest clonal revival, suggesting that the significant expansion of clonally enriched Vβ T_{EM} TILs might support the potential for improved anti-tumor activity of Compound 1 compared with CPI therapy.

In tumor-bearing mice, the 2-3-fold expansion of Vβ13 T cells in blood and tumor tissue was shown to be a determinant of the anti-tumor activity of mSTAR. Thus, the selective expansion of Vβ6/Vβ10 T cells at a similar magnitude in the blood of monkeys dosed with IV Compound 1 supported the potential for translation of the observed anti-tumor activity in mice into humans. Moreover, the relatively limited and delayed cytokine release observed in non-human primates and limited evidence of IL-2 driven toxicity suggested the potential for a more workable therapeutic index than observed with several published reports of anti-CD3 engagers.

To confirm the potential for anti-tumor activity. Compound 1 was assessed ex vivo in autologous human tumor organoids. In these organoids Compound 1 effectively expanded and converted tumor-resident, antigen-specific Vβ6/Vβ10 T cells into more productive effectors that potently killed tumor cells to a much greater extent than treatment with the anti-PD1 antibody pembrolizumab. This effect was further extended by in vitro studies demonstrating that human HPV-16 specific T cells from both healthy and cancer patients were boosted in the presence of Compound 1.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1.-119. (canceled)

120. A method of treating cancer in a human subject in need thereof comprising administering to the human subject a multifunctional molecule, wherein the multifunctional molecule comprises:
(a) a TCRβV6-binding moiety, and
(b) an interleukin-2 (IL-2) or a functional fragment or variant thereof; and
wherein the multifunctional molecule is administered to the human subject at a first dose of from about 0.001 mg/kg to about 10 mg/kg, thereby treating the cancer in the human subject.

121. The method of claim 120, wherein the method further comprises administering a second dose of the multifunctional molecule to the human subject.

122. The method of claim 120, wherein the first dose is from about 0.001 mg/kg to about 1 mg/kg.

123. The method of claim 120, wherein the first dose is from about 0.01 mg/kg to about 1 mg/kg.

124. The method of claim 123, wherein the first dose is from about 0.05 mg/kg to about 1 mg/kg,

125. The method of claim 124, wherein the first dose is about 0.08 mg/kg.

126. The method of claim 121, wherein the second dose is from about 0.001 mg/kg to about 1 mg/kg.

127. The method of claim 121, wherein the second dose is from about 0.01 mg/kg to about 1 mg/kg.

128. The method of claim 127, wherein the second dose is from about 0.05 mg/kg to about 1 mg/kg.

129. The method of claim 128, wherein the second dose is about 0.08 mg/kg.

130. The method of claim 121, wherein the second dose is administered at least 7 days after administration of the first dose.

131. The method of claim 121, wherein the second dose is administered at least 13 days after administration of the first dose.

132. The method of claim 121, wherein the second dose is administered about 14 days after administration of the first dose.

133. The method of claim 120, wherein the method comprises administering the multifunctional molecule to the human subject once every two weeks.

134. The method of claim 121, wherein the first dose is from about 0.01 mg/kg to about 1 mg/kg and the second dose is from about 0.01 mg/kg to about 1 mg/kg.

135. The method of claim 120, wherein the method comprises administering a dose of from about 0.01 mg/kg to about 1 mg/kg of the multifunctional molecule to the human subject once every two weeks.

136. The method of claim 120, wherein the method comprises administering the multifunctional molecule to the human subject once every week.

137. The method of claim 120, wherein the method comprises administering the multifunctional molecule via intravenous infusion.

138. The method of claim 120, wherein the human subject is at least 18 years old.

139. The method of claim 120, wherein the human subject:
(i) does not have a history of autoimmune disease;
(ii) does not have a major surgery or traumatic injury within 8 weeks before a first administration of the multifunctional molecule or the subject does not have an unhealed wound from surgery or injury;
(iii) is not treated with >10 mg per day of an immune-suppressive drug within 7 days prior to a first administration of the multifunctional molecule;
(iv) is not previously treated with a cytotoxic chemotherapy, a small molecule inhibitor, radiation, or an interventional radiology procedure with 2 weeks prior to a first administration of the multifunctional molecule;
(v) is not previously treated with a monoclonal antibody, an antibody-drug conjugate, a radioimmunoconjugate within 6 weeks prior to a first administration of the multifunctional molecule;
(vi) does not have an inflammatory process that is not resolved within 4 weeks before a first administration of the multifunctional molecule;
(vii) does not have a clinically significant pulmonary compromise; or
(viii) does not have an active viral, bacterial, or systemic fungal infection requiring parenteral treatment within 7 days of a first administration of the multifunctional molecule.

140. The method of claim 120, wherein the human subject is characterized as having a solid tumor, and wherein the solid tumor is selected from the group consisting of a high mutational burden (TMB-H) tumor, microsatellite instability/DNA mismatch repair (MSI-H/dMMR), a virally associated tumor, metastatic triple-negative breast cancer (mTNBC), relapsed and refractory epithelial ovarian cancer, metastatic castration-resistant prostate cancer (mCRPC), colorectal cancer (CRC), and non-small cell lung cancer (NSCLC).

141. The method of claim 120, wherein the method further comprises administrating at least one additional therapeutic agent or therapy to the human subject.

142. The method of claim 120, wherein the multifunctional molecule comprises a first polypeptide, a second polypeptide, and a third polypeptide,
wherein the first polypeptide, the second polypeptide and the third polypeptide are non-contiguous; and wherein:
(i) the first polypeptide comprises an Fc domain linked to a first portion of the TCRβV6-binding moiety, the first portion of the TCRβV6-binding moiety comprising a VH of the TCRβV6-binding moiety;
(ii) the second polypeptide comprises an Fc domain covalently linked to the IL-2 or functional fragment or functional variant thereof, and
(iii) the third polypeptide comprises a second portion of the TCRβV6-binding moiety, the second portion of the TCRβV6-binding moiety comprising a VL of the TCRβV6-binding moiety.

143. The method of claim 142, wherein
(a) the first polypeptide comprises a sequence with at least 90% sequence identity to SEQ ID NO: 3517, the second polypeptide comprises a sequence with at least 90% sequence identity to SEQ ID NO: 3521, and the third polypeptide comprises a sequence with at least 90% sequence identity to SEQ ID NO: 3518;
(b) the first polypeptide comprises a sequence with at least 90% sequence identity to SEQ ID NO: 4000, the second polypeptide comprises a sequence with at least 90% sequence identity to SEQ ID NO: 4002, and the third polypeptide comprises a sequence with at least 90% sequence identity to SEQ ID NO: 3518;
(c) the first polypeptide comprises a sequence with at least 90% sequence identity to SEQ ID NO: 4004, the second polypeptide comprises a sequence with at least 90% sequence identity to SEQ ID NO: 4030, and the third polypeptide comprises a sequence with at least 90% sequence identity to SEQ ID NO: 4005;
(d) the first polypeptide comprises a sequence with at least 90% sequence identity to SEQ ID NO: 4006, the second polypeptide comprises a sequence with at least 90% sequence identity to SEQ ID NO: 4007, and the third polypeptide comprises a sequence with at least 90% sequence identity to SEQ ID NO: 4005;
(e) the first polypeptide comprises a sequence with at least 90% sequence identity to SEQ ID NO: 4008, the second polypeptide comprises a sequence with at least 90% sequence identity to SEQ ID NO: 3521, and the third polypeptide comprises a sequence with at least 90% sequence identity to SEQ ID NO: 4009;
(f) the first polypeptide comprises a sequence with at least 90% sequence identity to SEQ ID NO: 4010, the second polypeptide comprises a sequence with at least 90% sequence identity to SEQ ID NO: 4003, and the third polypeptide comprises a sequence with at least 90% sequence identity to SEQ ID NO: 4009;
(g) the first polypeptide comprises a sequence with at least 90% sequence identity to SEQ ID NO: 4011, the second polypeptide comprises a sequence with at least 90% sequence identity to SEQ ID NO: 4013, and the third polypeptide comprises a sequence with at least 90% sequence identity to SEQ ID NO: 4012;
(h) the first polypeptide comprises a sequence with at least 90% sequence identity to SEQ ID NO: 4014, the second polypeptide comprises a sequence with at least 90% sequence identity to SEQ ID NO: 4015, and the third polypeptide comprises a sequence with at least 90% sequence identity to SEQ ID NO: 3012;
(i) the first polypeptide comprises a sequence with at least 90% sequence identity to SEQ ID NO: 4016, the second polypeptide comprises a sequence with at least 90% sequence identity to SEQ ID NO: 3521, and the third polypeptide comprises a sequence with at least 90% sequence identity to SEQ ID NO: 4017; or
(j) the first polypeptide comprises a sequence with at least 90% sequence identity to SEQ ID NO: 4018, the second polypeptide comprises a sequence with at least 90% sequence identity to SEQ ID NO: 4002, and the third polypeptide comprises a sequence with at least 90% sequence identity to SEQ ID NO: 4017.

144. The method of claim 120, wherein the multifunctional molecule comprises a first polypeptide and a second polypeptide,
wherein the first polypeptide and the second polypeptide are non-contiguous, and wherein:
(i) the first polypeptide comprises an Fc domain linked to the TCRβV6-binding moiety; and
(ii) the second polypeptide comprises an Fc domain covalently linked to the IL-2 or functional fragment or functional variant thereof.

145. The method of claim 144, wherein
(a) the first polypeptide comprises a sequence with at least 90% sequence identity to SEQ ID NO: 4019, and the second polypeptide comprises a sequence with at least 90% sequence identity to SEQ ID NO: 4020;
(b) the first polypeptide comprises a sequence with at least 90% sequence identity to SEQ ID NO: 4021, and the second polypeptide comprises a sequence with at least 90% sequence identity to SEQ ID NO: 4022;
(c) the first polypeptide comprises a sequence with at least 90% sequence identity to SEQ ID NO: 4023, and the second polypeptide comprises a sequence with at least 90% sequence identity to SEQ ID NO: 4024;
(d) the first polypeptide comprises a sequence with at least 90% sequence identity to SEQ ID NO: 4025, and the second polypeptide comprises a sequence with at least 90% sequence identity to SEQ ID NO: 4026; or
(e) the first polypeptide comprises a sequence with at least 90% sequence identity to SEQ ID NO: 4027, and the second polypeptide comprises a sequence with at least 90% sequence identity to SEQ ID NO: 4028.

146. The method of claim 120, wherein administering comprises administering a pharmaceutical composition comprising the multifunctional molecule, and wherein the pharmaceutical composition further comprises a pharmaceutically acceptable excipient, carrier or diluent.

147. The method of claim 146, wherein the pharmaceutical composition comprises a pharmaceutically acceptable diluent that is a saline solution.

148. The method of claim 146, wherein the pharmaceutical composition comprises a pharmaceutically acceptable diluent that is a 0.9% saline solution.

149. The method of claim 146, wherein the multifunctional molecule is present in the pharmaceutical composition at a concentration of from about 0.02 mg/mL to about 15 mg/mL

150. The method of claim 146, wherein the multifunctional molecule is present in the pharmaceutical composition at a concentration of from about 0.02 mg/mL to about 1.5 mg/mL.

151. A dose of a pharmaceutical composition comprising a multifunctional molecule, wherein the multifunctional molecule comprises:
(a) a TCRβV6-binding moiety, and
(b) an interleukin-2 (IL-2) or a functional fragment or variant thereof, and
wherein the dose is from about 0.001 mg/kg to about 10 mg/kg of the multifunctional molecule.

152. A pharmaceutical composition comprising a multifunctional molecule and a pharmaceutically acceptable diluent,
wherein the multifunctional molecule comprises:
(a) a TCRβV6-binding moiety, and
(b) an interleukin-2 (IL-2) or a functional fragment or variant thereof,
wherein the pharmaceutically acceptable diluent is a 0.9% saline solution; and
wherein the multifunctional molecule is present in the pharmaceutical composition at a concentration of from about 0.02 mg/mL to about 15 mg/mL.


SEQUENCE LISTING (4055 sequences)

SEQ ID NO: 1 - protein, 119 aa, synthetic construct
QVQLQQSGPELVKPGTSVKISCKASGYSFTTYYIHWVKQRPGQGLEWIGWFFPGSGNIKYNEKFKGKATLTADTSSSTAYMQLSSLTSEESAVYFCAGSYYSYDVLDYWGHGTTLTVSS

SEQ ID NO: 2 - protein, 107 aa, synthetic construct
DILMTQSQKFMSTSLGDRVSVSCKASQNVGINVVWHQQKPGQSPKALIYSSSHRYSGVPDRFTGSGSGTDFTLTINNVQSEDLAEYFCQQFKSYPLTFGAGTKLELK

SEQ ID NO: 3 - protein, 10 aa, synthetic construct
GYSFTTYYIH

SEQ ID NO: 4 - protein, 17 aa, synthetic construct
WFFPGSGNIKYNEKFKG

SEQ ID NO: 5 - protein, 10 aa, synthetic construct
SYYSYDVLDY

SEQ ID NO: 6 - protein, 11 aa, synthetic construct
KASQNVGINVV

SEQ ID NO: 7 - protein, 7 aa, synthetic construct
SSSHRYS

SEQ ID NO: 8 - protein, 9 aa, synthetic construct
QQFKSYPLT

SEQ ID NO: 9 - protein, 119 aa, synthetic construct
QVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSS

SEQ ID NO: 10 - protein, 107 aa, synthetic construct
DIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 11 - protein, 107 aa, synthetic construct
DIQMTQSPSSLSASVGDRVTITCKASQNVGINVVWHQQKPGKVPKALIYSSSHRYSGVPSRFSGSGSGTDFTLTISSLQPEDVATYFCQQFKSYPLTFGQGTKLEIK

SEQ ID NO: 12 - DNA, 357 bp, synthetic construct
  source 1..357: mol_type = other DNA
caggtgcagctggttcagtctggcgccgaagtgaagaaacctggctcctccgtgaaggtgtcctgcaaggcttccggctactccttcaccacctactacatccactgggtccgacaggcccctggacaaggattggaatggatgggctggttcttccccggctccggcaacatcaagtacaacgagaagttcaagggccgcgtgaccatcaccgccgacacctctacctctaccgcctacatggaactgtccagcctgagatctgaggacaccgccgtgtactactgcgccggctcctactactcttacgacgtgctggattactggggccagggcaccacagtgacagtgtcctct

SEQ ID NO: 13 - DNA, 321 bp, synthetic construct
  source 1..321: mol_type = other DNA
gacatccagatgacccagtctccatccttcctgtccgcctctgtgggcgacagagtgaccatcacatgcaaggcctctcagaacgtgggcatcaacgtcgtgtggcaccagcagaagcctggcaaggctcctaaggctctgatctactcctccagccaccggtactctggcgtgccctctagattttccggctctggctctggcaccgagtttaccctgacaatctccagcctgcagcctgaggacttcgccacctacttttgccagcagttcaagagctaccctctgacctttggccagggcaccaagctggaaatcaag

SEQ ID NO: 14 - DNA, 321 bp, synthetic construct
  source 1..321: mol_type = other DNA
gacatccagatgacccagtctccatcctctctgtccgcctctgtgggcgacagagtgaccatcacatgcaaggcctctcagaacgtgggcatcaacgtcgtgtggcaccagcagaaacctggcaaggtgcccaaggctctgatctactcctccagccacagatactccggcgtgccctctagattctccggctctggctctggcaccgactttaccctgacaatctccagcctgcagcctgaggacgtggccacctacttttgccagcagttcaagagctaccctctgacctttggccagggcaccaagctggaaatcaag

SEQ ID NO: 15 - protein, 15 aa, synthetic construct
RASKGVSTSGYSYLH

SEQ ID NO: 16 - protein, 7 aa, synthetic construct
LASYLES

SEQ ID NO: 17 - protein, 9 aa, synthetic construct
QHSRDLPLT

SEQ ID NO: 18 - protein, 5 aa, synthetic construct
NYYMY

SEQ ID NO: 19 - protein, 17 aa, synthetic construct
GINPSNGGTNFNEKFKN

SEQ ID NO: 20 - protein, 11 aa, synthetic construct
RDYRFDMGFDY

SEQ ID NO: 21-38 - skipped (18 entries)

SEQ ID NO: 39 - protein, 107 aa, synthetic construct [sequence omitted]

SEQ ID NO: 40 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 41 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 344 bp, Homo sapiens
  source 1..344: mol_type = genomic DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttactc

SEQ ID NO: 44 - protein, 114 aa, Homo sapiens
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSY

SEQ ID NO: 45 - protein, 5 aa, synthetic construct
TYYIH

SEQ ID NO: 46 - protein, 17 aa, synthetic construct
WFFPGSGNIKYNEKFKG

SEQ ID NO: 47 - protein, 10 aa, synthetic construct
SYYSYDVLDY

SEQ ID NO: 48 - protein, 7 aa, synthetic construct
GYSFTTY

SEQ ID NO: 49 - protein, 6 aa, synthetic construct
FPGSGN

SEQ ID NO: 50 - protein, 10 aa, synthetic construct
SYYSYDVLDY

SEQ ID NO: 51 - protein, 11 aa, synthetic construct
KASQNVGINVV

SEQ ID NO: 52 - protein, 7 aa, synthetic construct
SSSHRYS

SEQ ID NO: 53 - protein, 9 aa, synthetic construct
QQFKSYPLT

SEQ ID NO: 54 - protein, 11 aa, synthetic construct
KASQNVGINVV

SEQ ID NO: 55 - protein, 7 aa, synthetic construct
SSSHRYS

SEQ ID NO: 56 - protein, 9 aa, synthetic construct
QQFKSYPLT

SEQ ID NO: 57-68 - skipped (12 entries)

SEQ ID NO: 69 - protein, 592 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 70 - protein, 492 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 71 - protein, 479 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 72 - protein, 234 aa, synthetic construct
METDTLLLWVLLLWVPGSTGDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 453 aa, synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 74 - protein, 353 aa, synthetic construct
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 75 - protein, 340 aa, synthetic construct
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 76 - protein, 159 aa, synthetic construct
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 77-79 - skipped (3 entries)

SEQ ID NO: 80 - protein, 246 aa, synthetic construct
QVQLVQSGAEVKKPGSSVKVSCKASGTDFKLTYIHWVRQAPGQGLEWMGRVSPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 81 - protein, 107 aa, synthetic construct
DIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 82 - protein, 119 aa, synthetic construct
QVQLVQSGAEVKKPGSSVKVSCKASGTDFKLTYIHWVRQAPGQGLEWMGRVSPGSGNTKYNEKFKGRVTITADTSTSTAYMELSSLRSEDTAVYYCAGSYYSYDVLDYWGQGTTVTVSS

SEQ ID NO: 83 - protein, 246 aa, synthetic construct
QVQLVQSGAEVKKPGSSVKVSCKASGTDFDKIYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 84 - protein, 107 aa, synthetic construct; identical to SEQ ID NO: 81

SEQ ID NO: 85 - protein, 119 aa, synthetic construct
QVQLVQSGAEVKKPGSSVKVSCKASGTDFDKIYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 86 - protein, 246 aa, synthetic construct
QVQLVQSGAEVKKPGSSVKVSCKASGHDFRDFYIHWVRQAPGQGLEWMGRVYPGSGSYRYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 87 - protein, 107 aa, synthetic construct
DIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 88 - protein, 119 aa, synthetic construct
QVQLVQSGAEVKKPGSSVKVSCKASGHDFRDFYIHWVRQAPGQGLEWMGRVYPGSGSYRYNEKFKGRVTITADTSTSTAYMELSSLRSEDTAVYYCAGSYYSYDVLDYWGQGTTVTVSS

SEQ ID NO: 89 - protein, 246 aa, synthetic construct
QVQLVQSGAEVKKPGSSVKVSCKASGHDFKLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 90 - protein, 107 aa, synthetic construct
DIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 91 - protein, 119 aa, synthetic construct
QVQLVQSGAEVKKPGSSVKVSCKASGHDFKLTYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 92 - protein, 246 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVENKVAWHQQKPGKAPKALIYSSSHRYKGVPSRFSGSGSGTEFTLTISSLQPEDFATYFCQQFKSYPLTFGQGTKLEIK

SEQ ID NO: 93 - protein, 107 aa, synthetic construct
DIQMTQSPSFLSASVGDRVTITCKASQNVENKVAWHQQKPGKAPKALIYSSSHRYKGVPSRFSGSGSGTEFTLTISSLQPEDFATYFCQQFKSYPLTFGQGTKLEIK

SEQ ID NO: 94 - protein, 119 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAGSYYSYDVLDYWGQGTTVTVSS

SEQ ID NO: 95 - protein, 246 aa, synthetic construct
QVQLVQSGAEVKKPGSSVKVSCKASGTDFDKIYIHWVRQAPGQGLEWMGRIFAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 96 - protein, 107 aa, synthetic construct; identical to SEQ ID NO: 87

SEQ ID NO: 97 - protein, 119 aa, synthetic construct
QVQLVQSGAEVKKPGSSVKVSCKASGTDFDKIYIHWVRQAPGQGLEWMGRIFAGSGNTKYNEKFKGRVTITADTSTSTAYMELSSLRSEDTAVYYCAGSYYSYDVLDYWGQGTTVTVSS

SEQ ID NO: 98 - protein, 246 aa, synthetic construct
QVQLVQSGAEVKKPGSSVKVSCKASGHDFDKFYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYSGVPSRFSGSGSGTEFTLTISSLQPEDFATYFCQQFKSYPLTFGQGTKLEIK

SEQ ID NO: 99 - protein, 107 aa, synthetic construct
DIQMTQSPSFLSASVGDRVTITCKASQNVGNRVAWYQQKPGKAPKALIYSSSHRYSGVPSRFSGSGSGTEFTLTISSLQPEDFATYFCQQFKSYPLTFGQGTKLEIK

SEQ ID NO: 100 - protein, 119 aa, synthetic construct
QVQLVQSGAEVKKPGSSVKVSCKASGHDFDKFYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 101 - protein, 247 aa, synthetic construct
QVQLVQSGAEVKKPGSSVKVSCKASGHDFDKFYIHWVRQAPGQGLEWMGRIFAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIKS

SEQ ID NO: 102 - protein, 107 aa, synthetic construct
DIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 103 - protein, 119 aa, synthetic construct
QVQLVQSGAEVKKPGSSVKVSCKASGHDFDKFYIHWVRQAPGQGLEWMGRIFAGSGNVKYNEKFKGRVTITADTSTSTAYMELSSLRSEDTAVYYCAGSYYSYDVLDYWGQGTTVTVSS

SEQ ID NO: 104 - protein, 246 aa, synthetic construct
QVQLVQSGAEVKKPGSSVKVSCKASGTDFKLTYIHWVRQAPGQGLEWMGRVSPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 105 - protein, 107 aa, synthetic construct; identical to SEQ ID NO: 102

SEQ ID NO: 106 - protein, 119 aa, synthetic construct
QVQLVQSGAEVKKPGSSVKVSCKASGTDFKLTYIHWVRQAPGQGLEWMGRVSPGSGNVKYNEKFKGRVTITADTSTSTAYMELSSLRSEDTAVYYCAGSYYSYDVLDYWGQGTTVTVSS

SEQ ID NO: 107 - protein, 246 aa, synthetic construct
QVQLVQSGAEVKKPGSSVKVSCKASGTDFDKIYIHWVRQAPGQGLEWMGRVSAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 108 - protein, 107 aa, synthetic construct
DIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 109 - protein, 119 aa, synthetic construct
QVQLVQSGAEVKKPGSSVKVSCKASGTDFDKIYIHWVRQAPGQGLEWMGRVSAGSGNTKYNEKFKGRVTITADTSTSTAYMELSSLRSEDTAVYYCAGSYYSYDVLDYWGQGTTVTVSS

SEQ ID NO: 110 - protein, 246 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 111 - protein, 107 aa, synthetic construct; identical to SEQ ID NO: 90

SEQ ID NO: 112 - protein, 119 aa, synthetic construct; identical to SEQ ID NO: 94

SEQ ID NO: 113 - protein, 246 aa, synthetic construct
QVQLVQSGAEVKKPGSSVKVSCKASGTDFDKIYIHWVRQAPGQGLEWMGRISAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 114 - protein, 107 aa, synthetic construct; identical to SEQ ID NO: 87

SEQ ID NO: 115 - protein, 119 aa, synthetic construct
QVQLVQSGAEVKKPGSSVKVSCKASGTDFDKIYIHWVRQAPGQGLEWMGRISAGSGNTKYNEKFKGRVTITADTSTSTAYMELSSLRSEDTAVYYCAGSYYSYDVLDYWGQGTTVTVSS

SEQ ID NO: 116 - protein, 246 aa, synthetic construct
QVQLVQSGAEVKKPGSSVKVSCKASGTDFRLTYIHWVRQAPGQGLEWMGRVSPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNKVAWHQQKPGKAPKALIYSSSHRYKGVPSRFSGSGSGTEFTLTISSLQPEDFATYFCQQFKSYPLTFGQGTKLEIK

SEQ ID NO: 117 - protein, 107 aa, synthetic construct
DIQMTQSPSFLSASVGDRVTITCKASQNVDNKVAWHQQKPGKAPKALIYSSSHRYKGVPSRFSGSGSGTEFTLTISSLQPEDFATYFCQQFKSYPLTFGQGTKLEIK

SEQ ID NO: 118 - protein, 119 aa, synthetic construct
QVQLVQSGAEVKKPGSSVKVSCKASGTDFRLTYIHWVRQAPGQGLEWMGRVSPGSGNTKYNEKFKGRVTITADTSTSTAYMELSSLRSEDTAVYYCAGSYYSYDVLDYWGQGTTVTVSS

SEQ ID NO: 119 - protein, 246 aa, synthetic construct
QVQLVQSGAEVKKPGSSVKVSCKASGGTFRLTYIHWVRQAPGQGLEWMGRVYPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 120 - protein, 107 aa, synthetic construct; identical to SEQ ID NO: 87

SEQ ID NO: 121 - protein, 119 aa, synthetic construct
QVQLVQSGAEVKKPGSSVKVSCKASGGTFRLTYIHWVRQAPGQGLEWMGRVYPGSGNTKYNEKFKGRVTITADTSTSTAYMELSSLRSEDTAVYYCAGSYYSYDVLDYWGQGTTVTVSS

SEQ ID NO: 122 - protein, 246 aa, synthetic construct
QVQLVQSGAEVKKPGSSVKVSCKASGTDFRLTYIHWVRQAPGQGLEWMGRIFPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 123 - protein, 107 aa, synthetic construct; identical to SEQ ID NO: 87

SEQ ID NO: 124 - protein, 119 aa, synthetic construct
QVQLVQSGAEVKKPGSSVKVSCKASGTDFRLTYIHWVRQAPGQGLEWMGRIFPGSGNTKYNEKFKGRVTITADTSTSTAYMELSSLRSEDTAVYYCAGSYYSYDVLDYWGQGTTVTVSS

SEQ ID NO: 125 - protein, 246 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 126 - protein, 107 aa, synthetic construct; identical to SEQ ID NO: 81

SEQ ID NO: 127 - protein, 119 aa, synthetic construct; identical to SEQ ID NO: 94

SEQ ID NO: 128 - protein, 246 aa, synthetic construct
QVQLVQSGAEVKKPGSSVKVSCKASGGTFRLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 129 - protein, 107 aa, synthetic construct; identical to SEQ ID NO: 102

SEQ ID NO: 130 - protein, 119 aa, synthetic construct
QVQLVQSGAEVKKPGSSVKVSCKASGGTFRLTYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 131 - protein, 246 aa, synthetic construct
QVQLVQSGAEVKKPGSSVKVSCKASGGTFDKTYIHWVRQAPGQGLEWMGRISAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 132 - protein, 107 aa, synthetic construct; identical to SEQ ID NO: 87

SEQ ID NO: 133 - protein, 119 aa, synthetic construct
QVQLVQSGAEVKKPGSSVKVSCKASGGTFDKTYIHWVRQAPGQGLEWMGRISAGSGNTKYNEKFKGRVTITADTSTSTAYMELSSLRSEDTAVYYCAGSYYSYDVLDYWGQGTTVTVSS

SEQ ID NO: 134 - protein, 246 aa, synthetic construct
QVQLVQSGAEVKKPGSSVKVSCKASGHDFDKFYIHWVRQAPGQGLEWMGRISAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 135 - protein, 107 aa, synthetic construct; identical to SEQ ID NO: 87

SEQ ID NO: 136 - protein, 119 aa, synthetic construct
QVQLVQSGAEVKKPGSSVKVSCKASGHDFDKFYIHWVRQAPGQGLEWMGRISAGSGNTKYNEKFKGRVTITADTSTSTAYMELSSLRSEDTAVYYCAGSYYSYDVLDYWGQGTTVTVSS

SEQ ID NO: 137 - protein, 246 aa, synthetic construct
QVQLVQSGAEVKKPGSSVKVSCKASGTDFKLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNKVAWHQQKPGKAPKALIYSSSHRYKGVPSRFSGSGSGTEFTLTISSLQPEDFATYFCQQFKSYPLTFGQGTKLEIK

SEQ ID NO: 138 - protein, 107 aa, synthetic construct; identical to SEQ ID NO: 117

SEQ ID NO: 139 - protein, 119 aa, synthetic construct
QVQLVQSGAEVKKPGSSVKVSCKASGTDFKLTYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 140 - protein, 246 aa, synthetic construct
QVQLVQSGAEVKKPGSSVKVSCKASGHDFRLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 141 - protein, 107 aa, synthetic construct
DIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 142 - protein, 119 aa, synthetic construct
QVQLVQSGAEVKKPGSSVKVSCKASGHDFRLTYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 143 - protein, 246 aa, synthetic construct
QVQLVQSGAEVKKPGSSVKVSCKASGHDFHLWYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNKVAWHQQKPGKAPKALIYSSSHRYKGVPSRFSGSGSGTEFTLTISSLQPEDFATYFCQQFKSYPLTFGQGTKLEIK

SEQ ID NO: 144 - protein, 107 aa, synthetic construct; identical to SEQ ID NO: 117

SEQ ID NO: 145 - protein, 119 aa, synthetic construct
QVQLVQSGAEVKKPGSSVKVSCKASGHDFHLWYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 146 - protein, 246 aa, synthetic construct
QVQLVQSGAEVKKPGSSVKVSCKASGHDFHLWYIHWVRQAPGQGLEWMGRVFAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDKVAWYQQKPGKAPKALIYSSSHRYKGVPSRFSGSGSGTEFTLTISSLQPEDFATYFCQQFKSYPLTFGQGTKLEIK

SEQ ID NO: 147 - protein, 107 aa, synthetic construct
DIQMTQSPSFLSASVGDRVTITCKASQNVEDKVAWYQQKPGKAPKALIYSSSHRYKGVPSRFSGSGSGTEFTLTISSLQPEDFATYFCQQFKSYPLTFGQGTKLEIK

SEQ ID NO: 148 - protein, 119 aa, synthetic construct
QVQLVQSGAEVKKPGSSVKVSCKASGHDFHLWYIHWVRQAPGQGLEWMGRVFAGSGNTKYNEKFKGRVTITADTSTSTAYMELSSLRSEDTAVYYCAGSYYSYDVLDYWGQGTTVTVSS

SEQ ID NO: 149 - protein, 246 aa, synthetic construct
QVQLVQSGAEVKKPGSSVKVSCKASGTDFKLTYIHWVRQAPGQGLEWMGRIFPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 150 - protein, 107 aa, synthetic construct; identical to SEQ ID NO: 87

SEQ ID NO: 151 - protein, 119 aa, synthetic construct
QVQLVQSGAEVKKPGSSVKVSCKASGTDFKLTYIHWVRQAPGQGLEWMGRIFPGSGNTKYNEKFKGRVTITADTSTSTAYMELSSLRSEDTAVYYCAGSYYSYDVLDYWGQGTTVTVSS

SEQ ID NO: 152 - skipped

SEQ ID NO: 153 - protein, 246 aa, synthetic construct
QVQLVQSGAEVKKPGSSVKVSCKASGTDFKLTYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 154 - protein, 107 aa, synthetic construct; identical to SEQ ID NO: 108

SEQ ID NO: 155 - protein, 119 aa, synthetic construct
QVQLVQSGAEVKKPGSSVKVSCKASGTDFKLTYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 156 - protein, 246 aa, synthetic construct
QVQLVQSGAEVKKPGSSVKVSCKASGTDFKLTYIHWVRQAPGQGLEWMGRISPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 157 - protein, 107 aa, synthetic construct; identical to SEQ ID NO: 102

SEQ ID NO: 158 - protein, 119 aa, synthetic construct
QVQLVQSGAEVKKPGSSVKVSCKASGTDFKLTYIHWVRQAPGQGLEWMGRISPGSGNTKYNEKFKGRVTITADTSTSTAYMELSSLRSEDTAVYYCAGSYYSYDVLDYWGQGTTVTVSS

SEQ ID NO: 159 - protein, 246 aa, synthetic construct
QVQLVQSGAEVKKPGSSVKVSCKASGHDFHLWYIHWVRQAPGQGLEWMGRISPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWHQQKPGKAPKALIYSSSHRYKGVPSRFSGSGSGTEFTLTISSLQPEDFATYFCQQFKSYPLTFGQGTKLEIK

SEQ ID NO: 160 - protein, 107 aa, synthetic construct
DIQMTQSPSFLSASVGDRVTITCKASQNVGDRVAWHQQKPGKAPKALIYSSSHRYKGVPSRFSGSGSGTEFTLTISSLQPEDFATYFCQQFKSYPLTFGQGTKLEIK

SEQ ID NO: 161 - protein, 119 aa, synthetic construct
QVQLVQSGAEVKKPGSSVKVSCKASGHDFHLWYIHWVRQAPGQGLEWMGRISPGSGNVKYNEKFKGRVTITADTSTSTAYMELSSLRSEDTAVYYCAGSYYSYDVLDYWGQGTTVTVSS

SEQ ID NO: 162 - protein, 246 aa, synthetic construct
QVQLVQSGAEVKKPGSSVKVSCKASGHDFKLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 163 - protein, 107 aa, synthetic construct; identical to SEQ ID NO: 87

SEQ ID NO: 164 - protein, 119 aa, synthetic construct; identical to SEQ ID NO: 91

SEQ ID NO: 165 - protein, 246 aa, synthetic construct
QVQLVQSGAEVKKPGSSVKVSCKASGTDFHLWYIHWVRQAPGQGLEWMGRVFAGSGSYRYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 166 - protein, 107 aa, synthetic construct; identical to SEQ ID NO: 87

SEQ ID NO: 167 - protein, 119 aa, synthetic construct
QVQLVQSGAEVKKPGSSVKVSCKASGTDFHLWYIHWVRQAPGQGLEWMGRVFAGSGSYRYNEKFKGRVTITADTSTSTAYMELSSLRSEDTAVYYCAGSYYSYDVLDYWGQGTTVTVSS

SEQ ID NO: 168 - protein, 246 aa, synthetic construct
QVQLVQSGAEVKKPGSSVKVSCKASGTDFDKIYIHWVRQAPGQGLEWMGRISAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 169 - protein, 107 aa, synthetic construct; identical to SEQ ID NO: 141

SEQ ID NO: 170 - protein, 119 aa, synthetic construct; identical to SEQ ID NO: 115

SEQ ID NO: 171 - protein, 246 aa, synthetic construct
QVQLVQSGAEVKKPGSSVKVSCKASGTDFKLTYIHWVRQAPGQGLEWMGRISAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 172 - protein, 107 aa, synthetic construct; identical to SEQ ID NO: 81

SEQ ID NO: 173 - protein, 119 aa, synthetic construct
QVQLVQSGAEVKKPGSSVKVSCKASGTDFKLTYIHWVRQAPGQGLEWMGRISAGSGNTKYNEKFKGRVTITADTSTSTAYMELSSLRSEDTAVYYCAGSYYSYDVLDYWGQGTTVTVSS

SEQ ID NO: 174 - protein, 246 aa, synthetic construct
QVQLVQSGAEVKKPGSSVKVSCKASGTDFRLTYIHWVRQAPGQGLEWMGRISPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 175 - protein, 107 aa, synthetic construct; identical to SEQ ID NO: 108

SEQ ID NO: 176 - protein, 119 aa, synthetic construct
QVQLVQSGAEVKKPGSSVKVSCKASGTDFRLTYIHWVRQAPGQGLEWMGRISPGSGNTKYNEKFKGRVTITADTSTSTAYMELSSLRSEDTAVYYCAGSYYSYDVLDYWGQGTTVTVSS

SEQ ID NO: 177 - protein, 246 aa, synthetic construct
QVQLVQSGAEVKKPGSSVKVSCKASGHDFDKTYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 178 - protein, 107 aa, synthetic construct; identical to SEQ ID NO: 81

SEQ ID NO: 179 - protein, 119 aa, synthetic construct
QVQLVQSGAEVKKPGSSVKVSCKASGHDFDKTYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 180 - protein, 246 aa, synthetic construct
QVQLVQSGAEVKKPGSSVKVSCKASGHDFKLTYIHWVRQAPGQGLEWMGRVSPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWHQQKPGKAPKALIYSSSHRYKGVPSRFSGSGSGTEFTLTISSLQPEDFATYFCQQFKSYPLTFGQGTKLEIK

SEQ ID NO: 181 - protein, 107 aa, synthetic construct
DIQMTQSPSFLSASVGDRVTITCKASQNVEDRVAWHQQKPGKAPKALIYSSSHRYKGVPSRFSGSGSGTEFTLTISSLQPEDFATYFCQQFKSYPLTFGQGTKLEIK

SEQ ID NO: 182 - protein, 119 aa, synthetic construct
QVQLVQSGAEVKKPGSSVKVSCKASGHDFKLTYIHWVRQAPGQGLEWMGRVSPGSGNTKYNEKFKGRVTITADTSTSTAYMELSSLRSEDTAVYYCAGSYYSYDVLDYWGQGTTVTVSS

SEQ ID NO: 183 - protein, 246 aa, synthetic construct
QVQLVQSGAEVKKPGSSVKVSCKASGHDFDKTYIHWVRQAPGQGLEWMGRIYPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 184 - protein, 107 aa, synthetic construct; identical to SEQ ID NO: 141

SEQ ID NO: 185 - protein, 119 aa, synthetic construct
QVQLVQSGAEVKKPGSSVKVSCKASGHDFDKTYIHWVRQAPGQGLEWMGRIYPGSGNVKYNEKFKGRVTITADTSTSTAYMELSSLRSEDTAVYYCAGSYYSYDVLDYWGQGTTVTVSS

SEQ ID NO: 186 - protein, 246 aa, synthetic construct
QVQLVQSGAEVKKPGSSVKVSCKASGTDFDKTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 187 - protein, 107 aa, synthetic construct; identical to SEQ ID NO: 87

SEQ ID NO: 188 - protein, 119 aa, synthetic construct
QVQLVQSGAEVKKPGSSVKVSCKASGTDFDKTYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 189 - protein, 246 aa, synthetic construct
QVQLVQSGAEVKKPGSSVKVSCKASGTDFDKIYIHWVRQAPGQGLEWMGRISAGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 190 - protein, 107 aa, synthetic construct; identical to SEQ ID NO: 87

SEQ ID NO: 191 - protein, 119 aa, synthetic construct
QVQLVQSGAEVKKPGSSVKVSCKASGTDFDKIYIHWVRQAPGQGLEWMGRISAGSGNIKYNEKFKGRVTITADTSTSTAYMELSSLRSEDTAVYYCAGSYYSYDVLDYWGQGTTVTVSS

SEQ ID NO: 192 - protein, 246 aa, synthetic construct
QVQLVQSGAEVKKPGSSVKVSCKASGTDFDKIYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 193 - protein, 107 aa, synthetic construct; identical to SEQ ID NO: 102

SEQ ID NO: 194 - protein, 119 aa, synthetic construct; identical to SEQ ID NO: 85

SEQ ID NO: 195 - protein, 246 aa, synthetic construct
QVQLVQSGAEVKKPGSSVKVSCKASGGTFKLTYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 196 - protein, 107 aa, synthetic construct; identical to SEQ ID NO: 87

SEQ ID NO: 197 - protein, 119 aa, synthetic construct
QVQLVQSGAEVKKPGSSVKVSCKASGGTFKLTYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 198 - protein, 246 aa, synthetic construct
QVQLVQSGAEVKKPGSSVKVSCKASGTDFKLTYIHWVRQAPGQGLEWMGRISPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNRVAWHQQKPGKAPKALIYSSSHRYKGVPSRFSGSGSGTEFTLTISSLQPEDFATYFCQQFKSYPLTFGQGTKLEIK

SEQ ID NO: 199 - protein, 107 aa, synthetic construct
DIQMTQSPSFLSASVGDRVTITCKASQNVDNRVAWHQQKPGKAPKALIYSSSHRYKGVPSRFSGSGSGTEFTLTISSLQPEDFATYFCQQFKSYPLTFGQGTKLEIK

SEQ ID NO: 200 - protein, 119 aa, synthetic construct
QVQLVQSGAEVKKPGSSVKVSCKASGTDFKLTYIHWVRQAPGQGLEWMGRISPGSGNVKYNEKFKGRVTITADTSTSTAYMELSSLRSEDTAVYYCAGSYYSYDVLDYWGQGTTVTVSS

SEQ ID NO: 201 - protein, 246 aa, synthetic construct
QVQLVQSGAEVKKPGSSVKVSCKASGHDFDKFYIHWVRQAPGQGLEWMGRVSAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 202 - protein, 107 aa, synthetic construct; identical to SEQ ID NO: 90

SEQ ID NO: 203 - protein, 119 aa, synthetic construct
QVQLVQSGAEVKKPGSSVKVSCKASGHDFDKFYIHWVRQAPGQGLEWMGRVSAGSGNTKYNEKFKGRVTITADTSTSTAYMELSSLRSEDTAVYYCAGSYYSYDVLDYWGQGTTVTVSS

SEQ ID NO: 204 - protein, 246 aa, synthetic construct
QVQLVQSGAEVKKPGSSVKVSCKASGTDFDKFYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVVWYQQKPGKAPKALIYSSSHRYKGVPSRFSGSGSGTEFTLTISSLQPEDFATYFCQQFKSYPLTFGQGTKLEIK

SEQ ID NO: 205 - protein, 119 aa, synthetic construct
QVQLVQSGAEVKKPGSSVKVSCKASGTDFDKFYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 206 - protein, 246 aa, synthetic construct
QVQLVQSGAEVKKPGSSVKVSCKASGYSFTTYYIHWVRQAPGQGLEWMGWFSAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 207 - protein, 119 aa, synthetic construct
QVQLVQSGAEVKKPGSSVKVSCKASGYSFTTYYIHWVRQAPGQGLEWMGWFSAGSGNTKYNEKFKGRVTITADTSTSTAYMELSSLRSEDTAVYYCAVSYYSYDVLDYWGQGTTVTVSS

SEQ ID NO: 208 - protein, 246 aa, synthetic construct
QVQLVQSGAEVKKPGSSVKVSCKASGYSFTTYYIHWVRQAPGQGLEWMGWFSAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 209 - protein, 119 aa, synthetic construct
QVQLVQSGAEVKKPGSSVKVSCKASGYSFTTYYIHWVRQAPGQGLEWMGWFSAGSGNTKYNEKFKGRVTITADTSTSTAYMELSSLRSEDTAVYYCAGSYYSYDVLDYWGQGTTVTVSS

SEQ ID NO: 210 - protein, 246 aa, synthetic construct
QVQLVQSGAEVKKPGSSVKVSCKASGYSFTTYYIHWVRQAPGQGLEWMGWFFPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 211 - protein, 119 aa, synthetic construct
QVQLVQSGAEVKKPGSSVKVSCKASGYSFTTYYIHWVRQAPGQGLEWMGWFFPGSGNTKYNEKFKGRVTITADTSTSTAYMELSSLRSEDTAVYYCAGSYYSYDVLDYWGQGTTVTVSS

SEQ ID NO: 212 - protein, 246 aa, synthetic construct
QVQLVQSGAEVKKPGSSVKVSCKASGYSFTTYYIHWVRQAPGQGLEWMGWFSPGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 213 - protein, 119 aa, synthetic construct
QVQLVQSGAEVKKPGSSVKVSCKASGYSFTTYYIHWVRQAPGQGLEWMGWFSPGSGNTKYNEKFKGRVTITADTSTSTAYMELSSLRSEDTAVYYCAVSYYSYDVLDYWGQGTTVTVSS

SEQ ID NO: 214 - protein, 246 aa, synthetic construct
QVQLVQSGAEVKKPGSSVKVSCKASGYSFTTYYIHWVRQAPGQGLEWMGWFSPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 215 - protein, 119 aa, synthetic construct
QVQLVQSGAEVKKPGSSVKVSCKASGYSFTTYYIHWVRQAPGQGLEWMGWFSPGSGNTKYNEKFKGRVTITADTSTSTAYMELSSLRSEDTAVYYCAGSYYSYDVLDYWGQGTTVTVSS

SEQ ID NO: 216 - protein, 246 aa, synthetic construct
QVQLVQSGAEVKKPGSSVKVSCKASGYSFTTYYIHWVRQAPGQGLEWMGRIFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 217 - protein, 119 aa, synthetic construct
QVQLVQSGAEVKKPGSSVKVSCKASGYSFTTYYIHWVRQAPGQGLEWMGRIFPGSGNIKYNEKFKGRVTITADTSTSTAYMELSSLRSEDTAVYYCAGSYYSYDVLDYWGQGTTVTVSS

SEQ ID NO: 218 - protein, 246 aa, synthetic construct
QVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSIYSAGVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 219 - protein, 119 aa, synthetic construct
QVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSIYSAGVLDYWGQGTTVTVSS

SEQ ID NO: 220 - protein, 246 aa, synthetic construct
QVQLVQSGAEVKKPGSSVKVSCKASGYSFTLGYIHWVRQAPGQGLEWMGWFFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 221 - protein, 119 aa, synthetic construct
QVQLVQSGAEVKKPGSSVKVSCKASGYSFTLGYIHWVRQAPGQGLEWMGWFFPGSGNIKYNEKFKGRVTITADTSTSTAYMELSSLRSEDTAVYYCAGSYYSYDVLDYWGQGTTVTVSS

SEQ ID NO: 222 - protein, 246 aa, synthetic construct
QVQLVQSGAEVKKPGSSVKVSCKASGYSFRLTYIHWVRQAPGQGLEWMGWFFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 223 - protein, 119 aa, synthetic construct
QVQLVQSGAEVKKPGSSVKVSCKASGYSFRLTYIHWVRQAPGQGLEWMGWFFPGSGNIKYNEKFKGRVTITADTSTSTAYMELSSLRSEDTAVYYCAGSYYSYDVLDYWGQGTTVTVSS

SEQ ID NO: 224 - protein, 246 aa, synthetic construct
QVQLVQSGAEVKKPGSSVKVSCKASGYSFHNWYIHWVRQAPGQGLEWMGWFFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 225 - protein, 119 aa, synthetic construct
QVQLVQSGAEVKKPGSSVKVSCKASGYSFHNWYIHWVRQAPGQGLEWMGWFFPGSGNIKYNEKFKGRVTITADTSTSTAYMELSSLRSEDTAVYYCAGSYYSYDVLDYWGQGTTVTVSS

SEQ ID NO: 226-1099 - skipped (874 entries)

SEQ ID NO: 1100 - protein, 119 aa, synthetic construct; identical to SEQ ID NO: 9

SEQ ID NO: 1101 - protein, 106 aa, synthetic construct
QSVLTQPPSVSEAPRQRVTISCKASQNVGINVVWHQQLPGKAPKALIYSSSHRYSGVSDRFSGSGSGTSFSLAISGLQSEDEADYFCQQFKSYPLTFGTGTKVTVL

SEQ ID NO: 1102-1140 - skipped (39 entries)

SEQ ID NO: 1141 - protein, 5 aa, synthetic construct
SYAMS

SEQ ID NO: 1142 - protein, 17 aa, synthetic construct
HISNGGDYIYYADTVKG

SEQ ID NO: 1143 - protein, 12 aa, synthetic construct
PSYYSDPWFFDV

SEQ ID NO: 1144 - protein, 7 aa, synthetic construct
GFTFRSY

SEQ ID NO: 1145 - protein, 6 aa, synthetic construct
SNGGDY

SEQ ID NO: 1146 - protein, 10 aa, synthetic construct
GFTFRSYAMS

SEQ ID NO: 1147 - protein, 10 aa, synthetic construct
SAGSSVSFMH

SEQ ID NO: 1148 - protein, 7 aa, synthetic construct
DTSKLAS

SEQ ID NO: 1149 - protein, 9 aa, synthetic construct
LQGSGFPLT

SEQ ID NO: 1150 - protein, 6 aa, synthetic construct
GSSVSF

SEQ ID NO: 1151 - protein, 106 aa, synthetic construct
ENVLTQSPAIMSASPGEKVTMTCSAGSSVSFMHWYQQKSSTSPKLWIYDTSKLASGVPGRFSGSGSGNSFSLTISSMEAEDVAIYYCLQGSGFPLTFGSGTKLEIK

SEQ ID NO: 1152 - protein, 121 aa, synthetic construct
DVKLVESGEGLVKPGGSLKLSCAASGFTFRSYAMSWVRQTPEKRLEWVAHISNGGDYIYYADTVKGRFTISRDNARNTLYLQMSSLKSEDTAMYYCTRPSYYSDPWFFDVWGTGTTVTVSS

SEQ ID NO: 1153 - protein, 121 aa, synthetic construct
EVQLVESGGGLVQPGGSLRLSCAASGFTFRSYAMSWVRQAPGKGLEWVAHISNGGDYIYYADTVKGRFTISRDNAKNSLYLQMNSLRAEDTAVYYCTRPSYYSDPWFFDVWGQGTTVTVSS

SEQ ID NO: 1154 - protein, 121 aa, synthetic construct
QVQLVESGGGVVQPGRSLRLSCAASGFTFRSYAMSWVRQAPGKGLEWVAHISNGGDYIYYADTVKGRFTISRDNSKNTLYLQMSSLRAEDTAVYYCTRPSYYSDPWFFDVWGQGTTVTVSS

SEQ ID NO: 1155 - protein, 121 aa, synthetic construct
EVQLVESGGGLVQPGGSLRLSCAASGFTFRSYAMSWVRQAPGKGLEWVAHISNGGDYIYYADTVKGRFTISRDNSKNTLYLQMNSLRAEDTAVYYCTRPSYYSDPWFFDVWGQGTTVTVSS

SEQ ID NO: 1156 - protein, 121 aa, synthetic construct
QVQLVQSGSELKKPGASVKVSCKASGFTFRSYAMSWVRQAPGQGLEWVAHISNGGDYIYYADTVKGRFVISRDNSVNTLYLQISSLKAEDTAVYYCTRPSYYSDPWFFDVWGQGTTVTVSS

SEQ ID NO: 1157 - protein, 121 aa, synthetic construct
QVQLVQSGAEVKKPGASVKVSCKASGFTFRSYAMSWVRQAPGQRLEWVAHISNGGDYIYYADTVKGRFTITRDNSANTLYMELSSLRSEDTAVYYCTRPSYYSDPWFFDVWGQGTTVTVSS

SEQ ID NO: 1158 - protein, 106 aa, synthetic construct
ENVLTQSPATLSLSPGERATLSCSAGSSVSFMHWYQQKPGQAPKLLIYDTSKLASGIPARFSGSGSGNDFTLTISSLEPEDFAVYYCLQGSGFPLTFGQGTKLEIK

SEQ ID NO: 1159 - protein, 106 aa, synthetic construct
ENVLTQSPDFQSVTPKEKVTITCSAGSSVSFMHWYQQKPDQSPKLLIYDTSKLASGVPSRFSGSGSGNDFTLTINSLEAEDAATYYCLQGSGFPLTFGQGTKLEIK

SEQ ID NO: 1160 - protein, 106 aa, synthetic construct
DNQLTQSPSSLSASVGDRVTITCSAGSSVSFMHWYQQKPGKVPKLLIYDTSKLASGVPSRFSGSGSGNDFTLTISSLQPEDVATYYCLQGSGFPLTFGQGTKLEIK

SEQ ID NO: 1161 - protein, 106 aa, synthetic construct
ANQLTQSPSSLSASVGDRVTITCSAGSSVSFMHWYQQKPGKAPKLLIYDTSKLASGVPSRFSGSGSGNDFTLTISSLQPEDFATYYCLQGSGFPLTFGQGTKLEIK

SEQ ID NO: 1162 - protein, 106 aa, synthetic construct
DNVLTQSPDSLAVSLGERATINCSAGSSVSFMHWYQQKPGQPPKLLIYDTSKLASGVPDRFSGSGSGNDFTLTISSLQAEDVAVYYCLQGSGFPLTFGQGTKLEIK

SEQ ID NO: 1163-1287 - skipped (125 entries)

SEQ ID NO: 1288 - protein, 5 aa, synthetic construct
SYGMS

SEQ ID NO: 1289 - protein, 17 aa, synthetic construct
LISSGGSYTYYTDSVKG

SEQ ID NO: 1290 - protein, 8 aa, synthetic construct
HGGNFFDY

SEQ ID NO: 1291 - protein, 7 aa, synthetic construct
GFTFRSY

SEQ ID NO: 1292 - protein, 6 aa, synthetic construct
SSGGSY

SEQ ID NO: 1293 - protein, 10 aa, synthetic construct
GFTFRSYGMS

SEQ ID NO: 1294 - protein, 10 aa, synthetic construct
SVSSSVSYMH

SEQ ID NO: 1295 - protein, 7 aa, synthetic construct
DTSKLAS

SEQ ID NO: 1296 - protein, 10 aa, synthetic construct
QQWSSNPQYT

SEQ ID NO: 1297 - protein, 6 aa, synthetic construct
SSSVSY

SEQ ID NO: 1298-1308 - skipped (11 entries)

SEQ ID NO: 1309 - protein, 246 aa, synthetic construct
QVQLVQSGAEVKKPGSSVKVSCKASGHDFDKFYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 1310 - protein, 119 aa, synthetic construct; identical to SEQ ID NO: 94

SEQ ID NO: 1311 - protein, 119 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAVSYYSYDVLDYWGQGTTVTVSS

SEQ ID NO: 1312 - protein, 119 aa, synthetic construct
QVQLVQSGAEVKKPGSSVKVSCKASGHDFRLTYIHWVRQAPGQGLEWMGRISAGSGNVKYNEKFKGRVTITADTSTSTAYMELSSLRSEDTAVYYCAVSYYSYDVLDYWGQGTTVTVSS

SEQ ID NO: 1313 - protein, 107 aa, synthetic construct; identical to SEQ ID NO: 90

SEQ ID NO: 1314 - protein, 107 aa, synthetic construct; identical to SEQ ID NO: 141

SEQ ID NO: 1315-1325 - skipped (11 entries)

SEQ ID NO: 1326 - protein, 246 aa, synthetic construct
QVQLVQSGAEVKKPGSSVKVSCKASGHDFRLTYIHWVRQAPGQGLEWMGRVSAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGDRVVWHQQKPGKAPKALIYSSSHRYKGVPSRFSGSGSGTEFTLTISSLQPEDFATYFCQQFKSYPLTFGQGTKLEIK

SEQ ID NO: 1327 - protein, 246 aa, synthetic construct
QVQLVQSGAEVKKPGSSVKVSCKASGHDFRLTYIHWVRQAPGQGLEWMGRVSAGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGNRVVWHQQKPGKAPKALIYSSSHRYKGVPSRFSGSGSGTEFTLTISSLQPEDFATYFCQQFKSYPLTFGQGTKLEIK

SEQ ID NO: 1328 - protein, 246 aa, synthetic construct
QVQLVQSGAEVKKPGSSVKVSCKASGHDFRLTYIHWVRQAPGQGLEWMGRIYAGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVADRVVWHQQKPGKAPKALIYSSSHRYKGVPSRFSGSGSGTEFTLTISSLQPEDFATYFCQQFKSYPLTFGQGTKLEIK

SEQ ID NO: 1329 - protein, 246 aa, synthetic construct
QVQLVQSGAEVKKPGSSVKVSCKASGHDFKLTYIHWVRQAPGQGLEWMGRVSAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGDRVAWHQQKPGKAPKALIYSSSHRYKGVPSRFSGSGSGTEFTLTISSLQPEDFATYFCQQFKSYPLTFGQGTKLEIK

SEQ ID NO: 1330 - protein, 246 aa, synthetic construct
QVQLVQSGAEVKKPGSSVKVSCKASGHDFKLTYIHWVRQAPGQGLEWMGRVSAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVDNRVAWHQQKPGKAPKALIYSSSHRYKGVPSRFSGSGSGTEFTLTISSLQPEDFATYFCQQFKSYPLTFGQGTKLEIK

SEQ ID NO: 1331 - protein, 246 aa, synthetic construct
QVQLVQSGAEVKKPGSSVKVSCKASGHDFRLTYIHWVRQAPGQGLEWMGRVSAGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVADRVVWHQQKPGKAPKALIYSSSHRYKGVPSRFSGSGSGTEFTLTISSLQPEDFATYFCQQFKSYPLTFGQGTKLEIK

SEQ ID NO: 1332 - protein, 246 aa, synthetic construct
QVQLVQSGAEVKKPGSSVKVSCKASGHDFRLTYIHWVRQAPGQGLEWMGRVYAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVEDRVVWHQQKPGKAPKALIYSSSHRYKGVPSRFSGSGSGTEFTLTISSLQPEDFATYFCQQFKSYPLTFGQGTKLEIK

SEQ ID NO: 1333 - protein, 246 aa, synthetic construct
QVQLVQSGAEVKKPGSSVKVSCKASGHDFKLTYIHWVRQAPGQGLEWMGRVSAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVADRVVWHQQKPGKAPKALIYSSSHRYKGVPSRFSGSGSGTEFTLTISSLQPEDFATYFCQQFKSYPLTFGQGTKLEIK

SEQ ID NO: 1334 - protein, 246 aa, synthetic construct
QVQLVQSGAEVKKPGSSVKVSCKASGHDFKLTYIHWVRQAPGQGLEWMGRISAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGDRVVWHQQKPGKAPKALIYSSSHRYKGVPSRFSGSGSGTEFTLTISSLQPEDFATYFCQQFKSYPLTFGQGTKLEIK

SEQ ID NO: 1335 - protein, 246 aa, synthetic construct
QVQLVQSGAEVKKPGSSVKVSCKASGHDFKLTYIHWVRQAPGQGLEWMGRIYAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGDRVVWHQQKPGKAPKALIYSSSHRYKGVPSRFSGSGSGTEFTLTISSLQPEDFATYFCQQFKSYPLTFGQGTKLEIK

SEQ ID NO: 1336 - protein, 246 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 1337 - protein, 246 aa, synthetic construct
QVQLVQSGAEVKKPGSSVKVSCKASGHDFRLTYIHWVRQAPGQGLEWMGRISAGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 1338-1343 - skipped (6 entries)

SEQ ID NO: 1344 - protein, 119 aa, synthetic construct
QVQLVQSGAEVKKPGSSVKVSCKASGHDFRLTYIHWVRQAPGQGLEWMGRVSAGSGNTKYNEKFKGRVTITADTSTSTAYMELSSLRSEDTAVYYCAVSYYSYDVLDYWGQGTTVTVSS

SEQ ID NO: 1345 - skipped

SEQ ID NO: 1346 - protein, 119 aa, synthetic construct
QVQLVQSGAEVKKPGSSVKVSCKASGHDFRLTYIHWVRQAPGQGLEWMGRVSAGSGNVKYNEKFKGRVTITADTSTSTAYMELSSLRSEDTAVYYCAVSYYSYDVLDYWGQGTTVTVSS

SEQ ID NO: 1347 - protein, 107 aa, synthetic construct
DIQMTQSPSFLSASVGDRVTITCKASQNVGNRVVWHQQKPGKAPKALIYSSSHRYKGVPSRFSGSGSGTEFTLTISSLQPEDFATYFCQQFKSYPLTFGQGTKLEIK

SEQ ID NO: 1348 - protein, 119 aa, synthetic construct
QVQLVQSGAEVKKPGSSVKVSCKASGHDFRLTYIHWVRQAPGQGLEWMGRIYAGSGNVKYNEKFKGRVTITADTSTSTAYMELSSLRSEDTAVYYCAVSYYSYDVLDYWGQGTTVTVSS

SEQ ID NO: 1349 - protein, 107 aa, synthetic construct
DIQMTQSPSFLSASVGDRVTITCKASQNVADRVVWHQQKPGKAPKALIYSSSHRYKGVPSRFSGSGSGTEFTLTISSLQPEDFATYFCQQFKSYPLTFGQGTKLEIK

SEQ ID NO: 1350 - protein, 119 aa, synthetic construct
QVQLVQSGAEVKKPGSSVKVSCKASGHDFKLTYIHWVRQAPGQGLEWMGRVSAGSGNTKYNEKFKGRVTITADTSTSTAYMELSSLRSEDTAVYYCAVSYYSYDVLDYWGQGTTVTVSS

SEQ ID NO: 1351 - protein, 107 aa, synthetic construct; identical to SEQ ID NO: 160

SEQ ID NO: 1352 - skipped

SEQ ID NO: 1353 - protein, 107 aa, synthetic construct; identical to SEQ ID NO: 199

SEQ ID NO: 1354-1355 - skipped (2 entries)

SEQ ID NO: 1356 - protein, 119 aa, synthetic construct
QVQLVQSGAEVKKPGSSVKVSCKASGHDFRLTYIHWVRQAPGQGLEWMGRVYAGSGNTKYNEKFKGRVTITADTSTSTAYMELSSLRSEDTAVYYCAVSYYSYDVLDYWGQGTTVTVSS

SEQ ID NO: 1357 - protein, 107 aa, synthetic construct
DIQMTQSPSFLSASVGDRVTITCKASQNVEDRVVWHQQKPGKAPKALIYSSSHRYKGVPSRFSGSGSGTEFTLTISSLQPEDFATYFCQQFKSYPLTFGQGTKLEIK

SEQ ID NO: 1358-1359 - skipped (2 entries)

SEQ ID NO: 1360 - protein, 119 aa, synthetic construct
QVQLVQSGAEVKKPGSSVKVSCKASGHDFKLTYIHWVRQAPGQGLEWMGRISAGSGNTKYNEKFKGRVTITADTSTSTAYMELSSLRSEDTAVYYCAVSYYSYDVLDYWGQGTTVTVSS

SEQ ID NO: 1361 - protein, 107 aa, synthetic construct
DIQMTQSPSFLSASVGDRVTITCKASQNVGDRVVWHQQKPGKAPKALIYSSSHRYKGVPSRFSGSGSGTEFTLTISSLQPEDFATYFCQQFKSYPLTFGQGTKLEIK

SEQ ID NO: 1362 - protein, 119 aa, synthetic construct
QVQLVQSGAEVKKPGSSVKVSCKASGHDFKLTYIHWVRQAPGQGLEWMGRIYAGSGNTKYNEKFKGRVTITADTSTSTAYMELSSLRSEDTAVYYCAVSYYSYDVLDYWGQGTTVTVSS

SEQ ID NO: 1363-1364 - skipped (2 entries)

SEQ ID NO: 1365 - protein, 107 aa, synthetic construct
DIQMTQSPSFLSASVGDRVTITCRASQNVDNRLGWHQQKPGKAPKALIYSSSHRYKGVPSRFSGSGSGTEFTLTISSLQPEDFATYFCQQFKSYPLTFGQGTKLEIK

SEQ ID NO: 1366 - skipped

SEQ ID NO: 1367 - protein, 107 aa, synthetic construct
DIQMTQSPSFLSASVGDRVTITCKASQNVDNRVAWHQQKPGKAPKALIYAASSLQKGVPSRFSGSGSGTEFTLTISSLQPEDFATYFCQQFKSYPLTFGQGTKLEIK

SEQ ID NO: 1368 - skipped

SEQ ID NO: 1369 - protein, 107 aa, synthetic construct
DIQMTQSPSFLSASVGDRVTITCKASQNVDNRVAWHQQKPGKAPKALIYSSSHRYKGVPSRFSGSGSGTEFTLTISSLQPEDFATYFCLQHNSYPLTFGQGTKLEIK

SEQ ID NO: 1370 - protein, 119 aa, synthetic construct
QVQLVQSGAEVKKPGSSVKVSCKASGHDFKLTYIHWVRQAPGQGLEWMGRVSAGSGNVNYAQKFQGRVTITADTSTSTAYMELSSLRSEDTAVYYCAVSYYSYDVLDYWGQGTTVTVSS

SEQ ID NO: 1371-2190 - skipped (820 entries)

SEQ ID NO: 2191 - protein, 133 aa, synthetic construct [sequence omitted]

SEQ ID NO: 2192-2269 - skipped (78 entries)

SEQ ID NO: 2270 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 2271-2279 - skipped (9 entries)

SEQ ID NO: 2280 - protein, 133 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 2281-3182 - skipped (902 entries)

SEQ ID NO: 3183 - protein, 117 aa, synthetic construct
EVQLVESGGDLVKPGGSLKLSCAVSGFTFRSYGMSWVRQTPDKRLEWVALISSGGSYTYYTDSVKGRFTISRDNAKNTLYLQMSSLKSEDTAIYYCSRHGGNFFDYWGQGTTLTVSS

SEQ ID NO: 3184 - protein, 107 aa, synthetic construct
QIVLTQSPSIMSASPGEKVTMTCSVSSSVSYMHWYQQKSGTSPKRWIYDTSKLASGVPARFSGSGSGTSYSLTISSMEAEDAATYYCQQWSSNPQYTFGGGTKLEIK

SEQ ID NO: 3185 - protein, 107 aa, synthetic construct
DIVLTQSPAFLSVTPGEKVTITCSVSSSVSYMHWYQQKPDQAPKLLIYDTSKLASGVPSRFSGSGSGTDYTFTISSLEAEDAATYYCQQWSSNPQYTFGQGTKLEIK

SEQ ID NO: 3186 - protein, 107 aa, synthetic construct
AIQLTQSPSSLSASVGDRVTITCSVSSSVSYMHWYQQKPGKAPKLLIYDTSKLASGVPSRFSGSGSGTDYTLTISSLQPEDFATYYCQQWSSNPQYTFGQGTKLEIK

SEQ ID NO: 3187 - protein, 107 aa, synthetic construct
DIQLTQSPSFLSASVGDRVTITCSVSSSVSYMHWYQQKPGKAPKLLIYDTSKLASGVPSRFSGSGSGTEYTLTISSLQPEDFATYYCQQWSSNPQYTFGQGTKLEIK

SEQ ID NO: 3188 - protein, 107 aa, synthetic construct
DIQLTQSPSSLSASVGDRVTITCSVSSSVSYMHWYQQKPGKAPKLLIYDTSKLASGVPSRFSGSGSGTDYTLTISSLQPEDFATYYCQQWSSNPQYTFGQGTKLEIK

SEQ ID NO: 3189 - protein, 107 aa, synthetic construct
DIQLTQSPSSVSASVGDRVTITCSVSSSVSYMHWYQQKPGKAPKLLIYDTSKLASGVPSRFSGSGSGTDYTLTISSLQPEDFATYYCQQWSSNPQYTFGQGTKLEIK

SEQ ID NO: 3190 - protein, 107 aa, synthetic construct
DIQLTQSPSSLSASVGDRVTITCSVSSSVSYMHWYQQKPGKVPKLLIYDTSKLASGVPSRFSGSGSGTDYTLTISSLQPEDVATYYCQQWSSNPQYTFGQGTKLEIK

SEQ ID NO: 3191 - protein, 107 aa, synthetic construct
DIQLTQSPSSLSASVGDRVTITCSVSSSVSYMHWYQQKPGQAPKLLIYDTSKLASGVPSRFSGSGSGTDYTLTISSLQPEDVATYYCQQWSSNPQYTFGQGTKLEIK

SEQ ID NO: 3192 - protein, 107 aa, synthetic construct
EIVLTQSPDFQSVTPKEKVTITCSVSSSVSYMHWYQQKPDQSPKLLIYDTSKLASGVPSRFSGSGSGTDYTLTINSLEAEDAATYYCQQWSSNPQYTFGQGTKLEIK

SEQ ID NO: 3193 - protein, 107 aa, synthetic construct
AIRLTQSPFSLSASVGDRVTITCSVSSSVSYMHWYQQKPAKAPKLFIYDTSKLASGVPSRFSGSGSGTDYTLTISSLQPEDFATYYCQQWSSNPQYTFGQGTKLEIK

SEQ ID NO: 3194 - protein, 107 aa, synthetic construct
DIQLTQSPSSLSASVGDRVTITCSVSSSVSYMHWYQQKPGKAPKLLIYDTSKLASGVPSRFSGSGSGTDYTFTISSLQPEDIATYYCQQWSSNPQYTFGQGTKLEIK

SEQ ID NO: 3195 - protein, 107 aa, synthetic construct
EIVLTQSPATLSLSPGERATLSCSVSSSVSYMHWYQQKPGQAPKLLIYDTSKLASGIPARFSGSGSGTDYTLTISSLEPEDFAVYYCQQWSSNPQYTFGQGTKLEIK

SEQ ID NO: 3196 - protein, 107 aa, synthetic construct
DIQLTQSPSTLSASVGDRVTITCSVSSSVSYMHWYQQKPGKAPKLLIYDTSKLASGVPSRFSGSGSGTEYTLTISSLQPDDFATYYCQQWSSNPQYTFGQGTKLEIK

SEQ ID NO: 3197 - protein, 107 aa, synthetic construct
DIQLTQSPSSLSASVGDRVTITCSVSSSVSYMHWYQQKPGKTPKLLIYDTSKLASGIPSRFSGSGSGTDYTLTIRSLQPEDFATYYCQQWSSNPQYTFGQGTKLEIK

SEQ ID NO: 3198 - protein, 107 aa, synthetic construct
EIVLTQSPPTLSLSPGERVTLSCSVSSSVSYMHWYQQKPGQAPKLLIYDTSKLASGIPARFSGSGSGTDYTLTISSLQPEDFAVYYCQQWSSNPQYTFGQGTKLEIK

SEQ ID NO: 3199 - protein, 107 aa, synthetic construct
DIQLTQSPSSLSASVGDRVTITCSVSSSVSYMHWYQQKPGKAPKRLIYDTSKLASGVPSRFSGSGSGTEYTLTISSLQPEDFATYYCQQWSSNPQYTFGQGTKLEIK

SEQ ID NO: 3200 - protein, 107 aa, synthetic construct
EIVLTQSPATLSLSPGERATLSCSVSSSVSYMHWYQQKPGQAPKLLIYDTSKLASGIPARFSGSGPGTDYTLTISSLEPEDFAVYYCQQWSSNPQYTFGQGTKLEIK

SEQ ID NO: 3201 - protein, 107 aa, synthetic construct
EIVLTQSPATLSLSPGERATLSCSVSSSVSYMHWYQQKPGQAPKLLIYDTSKLASGIPARFSGSGSGTDYTLTISRLEPEDFAVYYCQQWSSNPQYTFGQGTKLEIK

SEQ ID NO: 3202 - protein, 107 aa, synthetic construct
EIVLTQSPATLSLSPGERATLSCSVSSSVSYMHWYQQKPGQAPKLLIYDTSKLASGIPARFSGSGSGTDYTLTISSLQPEDFAVYYCQQWSSNPQYTFGQGTKLEIK

SEQ ID NO: 3203 - protein, 107 aa, synthetic construct
EIVLTQSPATLSVSPGERATLSCSVSSSVSYMHWYQQKPGQAPKLLIYDTSKLASGIPARFSGSGSGTEYTLTISSLQSEDFAVYYCQQWSSNPQYTFGQGTKLEIK

SEQ ID NO: 3204 - protein, 107 aa, synthetic construct
EIVLTQSPATLSVSPGERATLSCSVSSSVSYMHWYQQKPGQAPKLLIYDTSKLASGIPARFSGSGSGTEYTLTISILQSEDFAVYYCQQWSSNPQYTFGQGTKLEIK

SEQ ID NO: 3205 - protein, 107 aa, synthetic construct
EIVLTQSPPTLSLSPGERVTLSCSVSSSVSYMHWYQQKPGQAPKLLIYDTSKLASSIPARFSGSGSGTDYTLTISSLQPEDFAVYYCQQWSSNPQYTFGQGTKLEIK

SEQ ID NO: 3206 - protein, 107 aa, synthetic construct
DIQLTQSPSSLSASVGDRVTITCSVSSSVSYMHWYQQKPGKAPKSLIYDTSKLASGVPSRFSGSGSGTDYTLTISSLQPEDFATYYCQQWSSNPQYTFGQGTKLEIK

SEQ ID NO: 3207 - protein, 107 aa, synthetic construct
DIQLTQSPSSLSASVGDRVTITCSVSSSVSYMHWYQQKPGKAPKRLIYDTSKLASGVPSRFSGSGSGTEYTLTISNLQPEDFATYYCQQWSSNPQYTFGQGTKLEIK

SEQ ID NO: 3208 - protein, 107 aa, synthetic construct
DIQLTQSPSAMSASVGDRVTITCSVSSSVSYMHWYQQKPGKVPKRLIYDTSKLASGVPSRFSGSGSGTEYTLTISSLQPEDFATYYCQQWSSNPQYTFGQGTKLEIK

SEQ ID NO: 3209 - protein, 107 aa, synthetic construct
EIVLTQSPATLSLSPGERATLSCSVSSSVSYMHWYQQKPGQAPKLLIYDTSKLASGIPDRFSGSGSGTDYTLTISRLEPEDFAVYYCQQWSSNPQYTFGQGTKLEIK

SEQ ID NO: 3210 - protein, 107 aa, synthetic construct
EIVLTQSPATLSLSPGERATLSCSVSSSVSYMHWYQQKPGLAPKLLIYDTSKLASGIPDRFSGSGSGTDYTLTISRLEPEDFAVYYCQQWSSNPQYTFGQGTKLEIK

SEQ ID NO: 3211 - protein, 107 aa, synthetic construct
EIVLTQSPGTLSLSPGERATLSCSVSSSVSYMHWYQQKPGQAPKLLIYDTSKLASGIPDRFSGSGSGTDYTLTISRLEPEDFAVYYCQQWSSNPQYTFGQGTKLEIK

SEQ ID NO: 3212 - protein, 107 aa, synthetic construct
DIQLTQSPSSLSASVGDRVTITCSVSSSVSYMHWYQQKPGKAPKSLIYDTSKLASGVPSKFSGSGSGTDYTLTISSLQPEDFATYYCQQWSSNPQYTFGQGTKLEIK

SEQ ID NO: 3213 - protein, 107 aa, synthetic construct
DIQLTQSPSSLSASVGDRVTITCSVSSSVSYMHWYQQKPEKAPKSLIYDTSKLASGVPSRFSGSGSGTDYTLTISSLQPEDFATYYCQQWSSNPQYTFGQGTKLEIK

SEQ ID NO: 3214 - protein, 107 aa, synthetic construct
DIVLTQSPDSLAVSLGERATINCSVSSSVSYMHWYQQKPGQPPKLLIYDTSKLASGVPDRFSGSGSGTDYTLTISSLQAEDVAVYYCQQWSSNPQYTFGQGTKLEIK

SEQ ID NO: 3215 - protein, 107 aa, synthetic construct
EIVLTQTPLSLSITPGEQASMSCSVSSSVSYMHWYLQKARPVPKLLIYDTSKLASGVPDRFSGSGSGTDYTLKISRVEAEDFGVYYCQQWSSNPQYTFGQGTKLEIK

SEQ ID NO: 3216 - protein, 107 aa, synthetic construct
EIVLTQTPLSLSITPGEQASISCSVSSSVSYMHWYLQKARPVPKLLIYDTSKLASGVPDRFSGSGSGTDYTLKISRVEAEDFGVYYCQQWSSNPQYTFGQGTKLEIK

SEQ ID NO: 3217 - protein, 107 aa, synthetic construct
DIVLTQSPLSLPVTPGEPASISCSVSSSVSYMHWYLQKPGQSPKLLIYDTSKLASGVPDRFSGSGSGTDYTLKISRVEAEDVGVYYCQQWSSNPQYTFGQGTKLEIK

SEQ ID NO: 3218 - protein, 107 aa, synthetic construct
DIVLTQSPLSLPVTLGQPASISCSVSSSVSYMHWYQQRPGQSPKRLIYDTSKLASGVPDRFSGSGSGTDYTLKISRVEAEDVGVYYCQQWSSNPQYTFGQGTKLEIK

SEQ ID NO: 3219 - protein, 107 aa, synthetic construct
DIVLTQTPLSLPVTPGEPASISCSVSSSVSYMHWYLQKPGQSPKLLIYDTSKLASGVPDRFSGSGSGTDYTLKISRVEAEDVGVYYCQQWSSNPQYTFGQGTKLEIK

SEQ ID NO: 3220 - protein, 107 aa, synthetic construct
DIVLTQTPLSLSVTPGQPASISCSVSSSVSYMHWYLQKPGQSPKLLIYDTSKLASGVPDRFSGSGSGTDYTLKISRVEAEDVGVYYCQQWSSNPQYTFGQGTKLEIK

SEQ ID NO: 3221 - protein, 107 aa, synthetic construct
DIVLTQTPLSLSVTPGQPASISCSVSSSVSYMHWYLQKPGQPPKLLIYDTSKLASGVPDRFSGSGSGTDYTLKISRVEAEDVGVYYCQQWSSNPQYTFGQGTKLEIK

SEQ ID NO: 3222 - protein, 107 aa, synthetic construct
DIQLIQSPSFLSASVGDRVSIICSVSSSVSYMHWYLQKPGKSPKLFIYDTSKLASGVSSRFSGRGSGTDYTLTIISLKPEDFAAYYCQQWSSNPQYTFGQGTKLEIK

SEQ ID NO: 3223 - protein, 107 aa, synthetic construct
DIVLTQTPLSSPVTLGQPASISCSVSSSVSYMHWYQQRPGQPPKLLIYDTSKLASGVPDRFSGSGAGTDYTLKISRVEAEDVGVYYCQQWSSNPQYTFGQGTKLEIK

SEQ ID NO: 3224 - protein, 107 aa, synthetic construct
EITLTQSPAFMSATPGDKVNISCSVSSSVSYMHWYQQKPGEAPKFIIYDTSKLASGIPPRFSGSGYGTDYTLTINNIESEDAAYYYCQQWSSNPQYTFGQGTKLEIK

SEQ ID NO: 3225 - protein, 117 aa, synthetic construct
EVQLVESGGGLVKPGGSLRLSCAVSGFTFRSYGMSWVRQAPGKGLEWVALISSGGSYTYYTDSVKGRFTISRDNSKNTLYLQMNSLKTEDTAVYYCSRHGGNFFDYWGQGTTVTVSS

SEQ ID NO: 3226 - protein, 117 aa, synthetic construct
EVQLVESGGALVKPGGSLRLSCAVSGFTFRSYGMSWVRQAPGKGLEWVALISSGGSYTYYTDSVKGRFTISRDNSKNTLYLQMNSLKTEDTAVYYCSRHGGNFFDYWGQGTTVTVSS

SEQ ID NO: 3227 - protein, 117 aa, synthetic construct
EVQLVESGGGLVQPGGSLRLSCAVSGFTFRSYGMSWVRQAPGKGLEWVALISSGGSYTYYTDSVKGRFTISRDNAKNTLYLQMNSLRAEDTAVYYCSRHGGNFFDYWGQGTTVTVSS

SEQ ID NO: 3228 - protein, 117 aa, synthetic construct
EVQLVESGGGLVQPGGSLRLSCAVSGFTFRSYGMSWVRQAPGKGLEWVALISSGGSYTYYTDSVKGRFTISRDNAKNSLYLQMNSLRAEDTAVYYCSRHGGNFFDYWGQGTTVTVSS

SEQ ID NO: 3229 - protein, 117 aa, synthetic construct
EVQLVESGGGLVQPGGSLRLSCAVSGFTFRSYGMSWVRQAPGKGLEWVALISSGGSYTYYTDSVKGRFTISRDNSKNSLYLQMNSLKTEDTAVYYCSRHGGNFFDYWGQGTTVTVSS

SEQ ID NO: 3230 - protein, 117 aa, synthetic construct
EVQLVESGGGLVQPGGSLRLSCAVSGFTFRSYGMSWVRQAPGKGLEWVALISSGGSYTYYTDSVKGRFTISRDNAKNSLYLQMNSLRAEDMAVYYCSRHGGNFFDYWGQGTTVTVSS

SEQ ID NO: 3231 - protein, 117 aa, synthetic construct
EVQLVESGGGLVQPGGSLRLSCAVSGFTFRSYGMSWVRQAPGKGLEWVALISSGGSYTYYTDSVKGQFTISRDNAKNTLYLQMNSLRAEDMAVYYCSRHGGNFFDYWGQGTTVTVSS

SEQ ID NO: 3232 - protein, 117 aa, synthetic construct
EVQLVESGGGLVKPGRSLRLSCTVSGFTFRSYGMSWVRQAPGKGLEWVALISSGGSYTYYTDSVKGRFTISRDNSKNILYLQMNSLKTEDTAVYYCSRHGGNFFDYWGQGTTVTVSS

SEQ ID NO: 3233 - protein, 117 aa, synthetic construct
EVQLVESGGGLVKPGGSLRLSCAVSGFTFRSYGMSWVRQAPGKGLEWVALISSGGSYTYYTDSVKGRFTISRDNAKNSLYLQMNSLRAEDTAVYYCSRHGGNFFDYWGQGTTVTVSS

SEQ ID NO: 3234 - protein, 117 aa, synthetic construct
EVQLVESGGGLVQPGGSLKLSCAVSGFTFRSYGMSWVRQASGKGLEWVALISSGGSYTYYTDSVKGRFTISRDNSKNTLYLQMNSLKTEDTAVYYCSRHGGNFFDYWGQGTTVTVSS

SEQ ID NO: 3235 - protein, 117 aa, synthetic construct
QVQLVESGGGVVQPGGSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3236 - protein, 117 aa, synthetic construct
QVQLVESGGGVVQPGRSLRLSCAVSGFTFRSYGMSWVRQAPGKGLEWVALISSGGSYTYYTDSVKGRFTISRDNSKNTLYLQMSSLRAEDTAVYYCSRHGGNFFDYWGQGTTVTVSS

SEQ ID NO: 3237 - protein, 117 aa, synthetic construct
EVQLVESGGGLVQPGGSLRLSCPVSGFTFRSYGMSWVRQAPGKGLEWVALISSGGSYTYYTDSVKGRFTISRDNANNSLYLQMNSLRAEDTAVYYCSRHGGNFFDYWGQGTTVTVSS

SEQ ID NO: 3238 - protein, 117 aa, synthetic construct
EVQLVESGGGLVQPGRSLRLSCTVSGFTFRSYGMSWVRQAPGKGLEWVALISSGGSYTYYTDSVKGRFTISRDNSKNILYLQMNSLKTEDTAVYYCSRHGGNFFDYWGQGTTVTVSS

SEQ ID NO: 3239 - protein, 117 aa, synthetic construct
EVQLVESGGGLVQPGPSLRLSCTVSGFTFRSYGMSWVRQAPGKGLEWVALISSGGSYTYYTDSVKGRFTISRDNSKNILYLQMNSLKTEDTAVYYCSRHGGNFFDYWGQGTTVTVSS

SEQ ID NO: 3240 - protein, 117 aa, synthetic construct
EVQLVESGGGLVQPGGSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3241 - protein, 117 aa, synthetic construct
EVQLVESGGGLVQPGGSLRLSCAVSGFTFRSYGMSWVRQAPGKGLEWVALISSGGSYTYYTDSVKGRFTISRDNAKNSLYLQMNSLRDEDTAVYYCSRHGGNFFDYWGQGTTVTVSS

SEQ ID NO: 3242 - protein, 117 aa, synthetic construct
QVQLVESGGGLVKPGGSLRLSCAVSGFTFRSYGMSWVRQAPGKGLEWVALISSGGSYTYYTDSVKGRFTISRDNAKNSLYLQMNSLRAEDTAVYYCSRHGGNFFDYWGQGTTVTVSS

SEQ ID NO: 3243 - protein, 117 aa, synthetic construct
QVQLVESGGGVVQPGRSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3244 - protein, 117 aa, synthetic construct
EVQLLESGGGLVQPGGSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3245 - protein, 117 aa, synthetic construct
EVQLVESGGGLVQPGGSLRLSCAVSGFTFRSYGMSWVRQAPGKGLEWVALISSGGSYTYYTDSVKGRFTISRHNSKNTLYLQMNSLRAEDTAVYYCSRHGGNFFDYWGQGTTVTVSS

SEQ ID NO: 3246 - protein, 117 aa, synthetic construct
EVQLVESGGGLIQPGGSLRLSCAVSGFTFRSYGMSWVRQPPGKGLEWVALISSGGSYTYYTDSVKGRFTISRDNSKNTLYLQMNSLRAEDTAVYYCSRHGGNFFDYWGQGTTVTVSS

SEQ ID NO: 3247 - protein, 117 aa, synthetic construct
EVQLVESGGGLIQPGGSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3248 - protein, 117 aa, synthetic construct
EVQLVESGGGLVQPGRSLRLSCAVSGFTFRSYGMSWVRQAPGKGLEWVALISSGGSYTYYTDSVKGRFTISRDNAKNSLYLQMNSLRAEDTALYYCSRHGGNFFDYWGQGTTVTVSS

SEQ ID NO: 3249 - protein, 117 aa, synthetic construct
QVQLVESGGGVVQPGRSLRLSCAVSGFTFRSYGMSWVRQAPGKGLEWVALISSGGSYTYYTDSVKGRFTISRDNSKNRLYLQMNSLRAEDTAVYYCSRHGGNFFDYWGQGTTVTVSS

SEQ ID NO: 3250 - protein, 117 aa, synthetic construct
QVQLVESGGGVVQPGRSLRLSCAVSGFTFRSYGMSWVRQAPGKGLEWVALISSGGSYTYYTDSVKGRFTISRDNSKNTLYLQMNSLRAEGTAVYYCSRHGGNFFDYWGQGTTVTVSS

SEQ ID NO: 3251 - protein, 117 aa, synthetic construct
QVQLVESGGGVVQPGRSLRLSCAVSGFTFRSYGMSWVRQAPGKGLEWVALISSGGSYTYYTDSVKGRFAISRDNSKNTLYLQMNSLRAEDTAVYYCSRHGGNFFDYWGQGTTVTVSS

SEQ ID NO: 3252 - protein, 117 aa, synthetic construct
QVQLVDSGGGVVQPGRSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3253 - protein, 117 aa, synthetic construct
EVQLVESGGGVVRPGGSLRLSCAVSGFTFRSYGMSWVRQAPGKGLEWVALISSGGSYTYYTDSVKGRFTISRDNAKNSLYLQMNSLRAEDTALYHCSRHGGNFFDYWGQGTTVTVSS

SEQ ID NO: 3254 - protein, 117 aa, synthetic construct
EVQLVESGGVVVQPGGSLRLSCAVSGFTFRSYGMSWVRQAPGKGLEWVALISSGGSYTYYTDSVKGRFTISRDNSKNSLYLQMNSLRAEDTALYYCSRHGGNFFDYWGQGTTVTVSS

SEQ ID NO: 3255 - protein, 117 aa, synthetic construct
EVQLVESGGGVVQPGGSLRLSCAVSGFTFRSYGMSWVRQAPGKGLEWVALISSGGSYTYYTDSVKGRFTISRDNSKNSLYLQMNSLRTEDTALYYCSRHGGNFFDYWGQGTTVTVSS

SEQ ID NO: 3256 - protein, 117 aa, synthetic construct
EVQLVESGGVVVQPGGSLRLSCAVSGFTFRSYGMSWVRQAPGKGLEWVALISSGGSYTYYTDSVKGRFTISRDNSKNSLYLQMNSLRTEDTALYYCSRHGGNFFDYWGQGTTVTVSS

SEQ ID NO: 3257 - protein, 117 aa, synthetic construct
EVQLVETGGGLIQPGGSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3258 - protein, 117 aa, synthetic construct
EVQLVESGGGLVQPGGSLRLSCAVSGFTFRSYGMSWVRQATGKGLEWVALISSGGSYTYYTDSVKGRFTISRENAKNSLYLQMNSLRAGDTAVYYCSRHGGNFFDYWGQGTTVTVSS

SEQ ID NO: 3259 - protein, 117 aa, synthetic construct
EVQLVESRGVLVQPGGSLRLSCAVSGFTFRSYGMSWVRQAPGKGLEWVALISSGGSYTYYTDSVKGRFTISRDNSKNTLHLQMNSLRAEDTAVYYCSRHGGNFFDYWGQGTTVTVSS

SEQ ID NO: 3260 - protein, 117 aa, synthetic construct
EVQLVESGGGLVQPGRSLRLSCAVSGFTFRSYGMSWVRQAPGKGLEWVALISSGGSYTYYTDSVKGRFTISRDNAKNSLYLQMNSLRAEDMALYYCSRHGGNFFDYWGQGTTVTVSS

SEQ ID NO: 3261 - protein, 117 aa, synthetic construct
QVQLVESGGGLVQPGGSLRLSCSVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3262 - protein, 117 aa, synthetic construct
EVQLVESGGGLVQPGGSLRLSCSVSGFTFRSYGMSWVRQAPGKGLEWVALISSGGSYTYYTDSVKGRFTISRDNSKNTLYLQMSSLRAEDTAVYYCSRHGGNFFDYWGQGTTVTVSS

SEQ ID NO: 3263 - protein, 117 aa, synthetic construct
QVQLVESGGGVVQPGRSLRLSCAVSGFTFRSYGMSWVRQAPGKGLEWVALISSGGSYTYYTDSVKGRFTISRDNSTNTLFLQMNSLRAEDTAVYYCSRHGGNFFDYWGQGTTVTVSS

SEQ ID NO: 3264 - protein, 117 aa, synthetic construct
QVQLLESGGGLVKPGGSLRLSCAVSGFTFRSYGMSWVRQAPGKGLEWVALISSGGSYTYYTDSVKGRFTISRDNAKNSLYLQMNSLRAEDTAVYYCSRHGGNFFDYWGQGTTVTVSS

SEQ ID NO: 3265 - protein, 117 aa, synthetic construct
EVQLVESGEGLVQPGGSLRLSCAVSGFTFRSYGMSWVRQAPGKGLEWVALISSGGSYTYYTDSVKGRFTISRDNSKNTLYLQMGSLRAEDMAVYYCSRHGGNFFDYWGQGTTVTVSS

SEQ ID NO: 3266 - protein, 117 aa, synthetic construct
EVQLVESGGGLVQPGGSLRLSCAVSGFTFRSYGMSWVRQAPGKGLEWVALISSGGSYTYYTDSVKGRFTISRDNSKNTLYLQMGSLRAEDMAVYYCSRHGGNFFDYWGQGTTVTVSS

SEQ ID NO: 3267 - protein, 117 aa, synthetic construct
EVQLVESGGGLVQPGGSLRLSCSVSGFTFRSYGMSWVRQAPGKGLEWVALISSGGSYTYYTDSVKGRFTISRDNSKNTLYVQMSSLRAEDTAVYYCSRHGGNFFDYWGQGTTVTVSS

SEQ ID NO: 3268 - protein, 117 aa, synthetic construct
EVQLVESGGGLVQPGGSLRLSCAVSGFTFRSYGMSWVRQAPGKGLEWVALISSGGSYTYYTDSVKGRFIISRDNSRNSLYLQKNRRRAEDMAVYYCSRHGGNFFDYWGQGTTVTVSS

SEQ ID NO: 3269 - protein, 117 aa, synthetic construct
EVQLVESGGGLVQPGGSLRLSCAVSGFTFRSYGMSWVHQAPGKGLEWVALISSGGSYTYYTDSVKGRFIISRDNSRNTLYLQTNSLRAEDTAVYYCSRHGGNFFDYWGQGTTVTVSS

SEQ ID NO: 3270 - protein, 117 aa, synthetic construct
EVHLVESGGGLVQPGGALRLSCAVSGFTFRSYGMSWVRQATGKGLEWVALISSGGSYTYYTDSVKGRFTISRENAKNSLYLQMNSLRAGDTAVYYCSRHGGNFFDYWGQGTTVTVSS

SEQ ID NO: 3271 - protein, 117 aa, synthetic construct
EVQLVESGGGLVQPRGSLRLSCAVSGFTFRSYGMSWVRQAPGKGLEWVALISSGGSYTYYTDSVKGRFTISRDNSKNTLYLQMNNLRAEGTAVYYCSRHGGNFFDYWGQGTTVTVSS

SEQ ID NO: 3272 - protein, 117 aa, synthetic construct
EVQLVESGGGLVQPRGSLRLSCAVSGFTFRSYGMSWVRQAPGKGLEWVALISSGGSYTYYTDSVKGRFTISRDNSKNTLYLQMNNLRAEGTAAYYCSRHGGNFFDYWGQGTTVTVSS

SEQ ID NO: 3273 - protein, 117 aa, synthetic construct
QVQLVQSGAEVKKPGASVKVSCKVSGFTFRSYGMSWVRQAPGKGLEWVALISSGGSYTYYTDSVKGRFTITRDNSTNTLYMELSSLRSEDTAVYYCSRHGGNFFDYWGQGTTVTVSS

SEQ ID NO: 3274 - protein, 117 aa, synthetic construct
QVQLVQSGSELKKPGASVKVSCKVSGFTFRSYGMSWVRQAPGQGLEWVALISSGGSYTYYTDSVKGRFVISRDNSVNTLYLQISSLKAEDTAVYYCSRHGGNFFDYWGQGTTVTVSS

SEQ ID NO: 3275-3277 - skipped (3 entries)

SEQ ID NO: 3278 - protein, 469 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3279 - protein, 234 aa, synthetic construct
METDTLLLWVLLLWVPGSTGDIQMTQSPSSLSASVGDRVTITCKASQNVGINVVWHQQKPGKVPKALIYSSSHRYSGVPSRFSGSGSGTDFTLTISSLQPEDVATYFCQQFKSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3280-3306 - skipped (27 entries)

SEQ ID NO: 3307 - protein, 5 aa, synthetic construct
GGGGS

SEQ ID NO: 3308 - protein, 10 aa, synthetic construct
GGGGSGGGGS

SEQ ID NO: 3309 - protein, 15 aa, synthetic construct
GGGGSGGGGSGGGGS

SEQ ID NO: 3310 - protein, 17 aa, synthetic construct
DVPSGPGGGGGSGGGGS

SEQ ID NO: 3311-3313 - skipped (3 entries)

SEQ ID NO: 3314 - protein, 14 aa, synthetic construct
AEAAAKEAAAKAAA

SEQ ID NO: 3315 - protein, 19 aa, synthetic construct
AEAAAKEAAAKEAAAKAAA

SEQ ID NO: 3316 - protein, 24 aa, synthetic construct
AEAAAKEAAAKEAAAKEAAAKAAA

SEQ ID NO: 3317 - protein, 29 aa, synthetic construct
AEAAAKEAAAKEAAAKEAAAKEAAAKAAA

SEQ ID NO: 3318-3436 - skipped (119 entries)

SEQ ID NO: 3437 - protein, 27 aa, synthetic construct
  VARIANT 12..26: note = EAAAK repeats may be deleted
AEAAAKEAAAKEAAAKEAAAKEAAAKA

SEQ ID NO: 3438-3516 - skipped (79 entries)

SEQ ID NO: 3517 - protein, 449 aa, synthetic construct
QVQLVQSGAEVKKPGSSVKVSCKASGHDFRLTYIHWVRQAPGQGLEWMGRVSAGSGNVKYNEKFKGRVTITADTSTSTAYMELSSLRSEDTAVYYCAVSYYSYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3518 - protein, 214 aa, synthetic construct
DIQMTQSPSFLSASVGDRVTITCKASQNVADRVVWHQQKPGKAPKALIYSSSHRYKGVPSRFSGSGSGTEFTLTISSLQPEDFATYFCQQFKSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3519-3520 - skipped (2 entries)

SEQ ID NO: 3521 - protein, 370 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3522-3525 - skipped (4 entries)

SEQ ID NO: 3526 - protein, 220 aa, synthetic construct
DIVMSQSPSSLAVSVGEKVTMSCKSSQSLLYSGNQKNYLAWYQQKPGQSPKLLIYWASTRESGVPDRFTGSGSGTDFTLTINSVKAEDLTVYYCQQYYGYPRTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3527 - protein, 113 aa, synthetic construct
DIVMSQSPSSLAVSVGEKVTMSCKSSQSLLYSGNQKNYLAWYQQKPGQSPKLLIYWASTRESGVPDRFTGSGSGTDFTLTINSVKAEDLTVYYCQQYYGYPRTFGGGTKVEIK

SEQ ID NO: 3528 - protein, 107 aa, synthetic construct
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3529 - protein, 452 aa, synthetic construct
QVQLQQSGTELMKPGASVKISCKASGYTFSNYWIEWIKQRPGHGLEWVGEILPGAGPTNYNEKFKGKATFTADSSSNTAYMQLSSLTSEDSAVYYCARTDYDYDWFAYWGQGTLVTVSAAKTTAPSVYPLAPVSGDTTGSSVTLGCLVKGYFPEPVTLTWNSGSLSSGVHTFPAVLQSDLYTLSSSVTVTSSTWPSQSITCNVAHPASSTKVDKKIEPKSCDKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVCVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPGKEPEA

SEQ ID NO: 3530 - protein, 192 aa, synthetic construct
QVQLQQSGTELMKPGASVKISCKASGYTFSNYWIEWIKQRPGHGLEWVGEILPGAGPTNYNEKFKGKATFTADSSSNTAYMQLSSLTSEDSAVYYCARTDYDYDWFAYWGQGTLVTVSAAKTTAPSVYPLAPVSGDTTGSSVTLGCLVKGYFPEPVTLTWNSGSLSSGVHTFPAVLQSDLYTLSSSVTVTSS

SEQ ID NO: 3531 - protein, 256 aa, synthetic construct
TWPSQSITCNVAHPASSTKVDKKIEPKSCDKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVCVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPGK

SEQ ID NO: 3532 - protein, 383 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSDKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPCEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGKGGGGSHHHHHHHH

SEQ ID NO: 3533 - protein, 227 aa, synthetic construct
DKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPCEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3534 - protein, 13 aa, synthetic construct
GGGGSHHHHHHHH

SEQ ID NO: 3535 - protein, 368 aa, synthetic construct
DIVMSQSPSSLAVSVGEKVTMSCKSSQSLLYSGNQKNYLAWYQQKPGQSPKLLIYWASTRESGVPDRFTGSGSGTDFTLTINSVKAEDLTVYYCQQYYGYPRTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 3536 - protein, 452 aa, synthetic construct
QVQLQQSGTELMKPGASVKISCKASGYTFSNYWIEWIKQRPGHGLEWVGEILPGAGPTNYNEKFKGKATFTADSSSNTAYMQLSSLTSEDSAVYYCARTDYDYDWFAYWGQGTLVTVSAAKTTAPSVYPLAPVSGDTTGSSVTLGCLVKGYFPEPVTLTWNSGSLSSGVHTFPAVLQSDLYTLSSSVTVTSSTWPSQSITCNVAHPASSTKVDKKIEPKSCDKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKEPEA

SEQ ID NO: 3537 - protein, 260 aa, synthetic construct
TWPSQSITCNVAHPASSTKVDKKIEPKSCDKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKEPEA

SEQ ID NO: 3538-3546 - skipped (9 entries)

SEQ ID NO: 3547 - protein, 4 aa, synthetic construct
EPEA

SEQ ID NO: 3548 - protein, 448 aa, synthetic construct
QVQLQQSGTELMKPGASVKISCKASGYTFSNYWIEWIKQRPGHGLEWVGEILPGAGPTNYNEKFKGKATFTADSSSNTAYMQLSSLTSEDSAVYYCARTDYDYDWFAYWGQGTLVTVSAAKTTAPSVYPLAPVSGDTTGSSVTLGCLVKGYFPEPVTLTWNSGSLSSGVHTFPAVLQSDLYTLSSSVTVTSSTWPSQSITCNVAHPASSTKVDKKIEPKSCDKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVCVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPGK

SEQ ID NO: 3549 - protein, 370 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSDKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPCEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3550-3642 - skipped (93 entries)

SEQ ID NO: 3643 - protein, 16 aa, synthetic construct
GGGGSGGGGSGGGGGS

SEQ ID NO: 3644 - protein, 107 aa, synthetic construct [sequence omitted]

SEQ ID NO: 3645 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3646 - protein, 334 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKEPEA

SEQ ID NO: 3647 - protein, 330 aa, synthetic construct; identical to SEQ ID NO: 41

SEQ ID NO: 3648 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3649 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3650 - protein, 10 aa, synthetic construct
GHDFRLTYIH

SEQ ID NO: 3651 - protein, 17 aa, synthetic construct
RVSAGSGNVKYNEKFKG

SEQ ID NO: 3652 - protein, 11 aa, synthetic construct
KASQNVGNRVV

SEQ ID NO: 3653 - protein, 7 aa, synthetic construct
SSSHRYK

SEQ ID NO: 3654 - protein, 17 aa, synthetic construct
RIYAGSGNVKYNEKFKG

SEQ ID NO: 3655 - protein, 11 aa, synthetic construct
KASQNVADRVV

SEQ ID NO: 3656 - protein, 10 aa, synthetic construct
GHDFKLTYIH

SEQ ID NO: 3657 - protein, 17 aa, synthetic construct
RVSAGSGNTKYNEKFKG

SEQ ID NO: 3658 - protein, 11 aa, synthetic construct
KASQNVGDRVA

SEQ ID NO: 3659 - protein, 17 aa, synthetic construct
RVYAGSGNTKYNEKFKG

SEQ ID NO: 3660 - protein, 11 aa, synthetic construct
KASQNVEDRVV

SEQ ID NO: 3661 - protein, 17 aa, synthetic construct
RISAGSGNTKYNEKFKG

SEQ ID NO: 3662 - protein, 11 aa, synthetic construct
KASQNVGDRVV

SEQ ID NO: 3663 - protein, 17 aa, synthetic construct
RIYAGSGNTKYNEKFKG

SEQ ID NO: 3664 - protein, 9 aa, synthetic construct
LQHNSYPLT

SEQ ID NO: 3665 - protein, 17 aa, synthetic construct
RVSAGSGNVNYAQKFQG

SEQ ID NO: 3666 - protein, 11 aa, synthetic construct
RASQNVDNRLG

SEQ ID NO: 3667 - protein, 11 aa, synthetic construct
KASQNVDNRVA

SEQ ID NO: 3668 - protein, 7 aa, synthetic construct
AASSLQK

SEQ ID NO: 3669-3999 - skipped (331 entries)

SEQ ID NO: 4000 - protein, 447 aa, synthetic construct
QVQLVQSGAEVKKPGSSVKVSCKASGHDFRLTYIHWVRQAPGQGLEWMGRVSAGSGNVKYNEKFKGRVTITADTSTSTAYMELSSLRSEDTAVYYCAVSYYSYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 4001 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 4002 - protein, 368 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 4003 - protein, 368 aa, synthetic construct; identical to SEQ ID NO: 4002

SEQ ID NO: 4004 - protein, 449 aa, synthetic construct
QVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 4005 - protein, 214 aa, synthetic construct
DIQMTQSPSSLSASVGDRVTITCKASQNVGINVVWHQQKPGKVPKALIYSSSHRYSGVPSRFSGSGSGTDFTLTISSLQPEDVATYFCQQFKSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4006 - protein, 447 aa, synthetic construct
QVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 4007 - protein, 368 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 4008 - protein, 449 aa, synthetic construct
QVQLVQSGAEVKKPGSSVKVSCKASGTDFDKIYIHWVRQAPGQGLEWMGRVSAGSGNTKYNEKFKGRVTITADTSTSTAYMELSSLRSEDTAVYYCAGSYYSYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 4009 - protein, 214 aa, synthetic construct
DIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4010 - protein, 447 aa, synthetic construct
QVQLVQSGAEVKKPGSSVKVSCKASGTDFDKIYIHWVRQAPGQGLEWMGRVSAGSGNTKYNEKFKGRVTITADTSTSTAYMELSSLRSEDTAVYYCAGSYYSYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 4011 - protein, 449 aa, synthetic construct
QVQLVQSGAEVKKPGSSVKVSCKASGHDFKLTYIHWVRQAPGQGLEWMGRVSPGSGNTKYNEKFKGRVTITADTSTSTAYMELSSLRSEDTAVYYCAGSYYSYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 4012 - protein, 214 aa, synthetic construct
DIQMTQSPSFLSASVGDRVTITCKASQNVEDRVAWHQQKPGKAPKALIYSSSHRYKGVPSRFSGSGSGTEFTLTISSLQPEDFATYFCQQFKSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4013 - protein, 370 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4014 - protein, 447 aa, synthetic construct
QVQLVQSGAEVKKPGSSVKVSCKASGHDFKLTYIHWVRQAPGQGLEWMGRVSPGSGNTKYNEKFKGRVTITADTSTSTAYMELSSLRSEDTAVYYCAGSYYSYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 4015 - protein, 368 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 4016 - protein, 449 aa, synthetic construct
QVQLVQSGAEVKKPGSSVKVSCKASGTDFDKTYIHWVRQAPGQGLEWMGRISAGSGNVKYNEKFKGRVTITADTSTSTAYMELSSLRSEDTAVYYCAGSYYSYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 4017 - protein, 214 aa, synthetic construct
DIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4018 - protein, 447 aa, synthetic construct
QVQLVQSGAEVKKPGSSVKVSCKASGTDFDKTYIHWVRQAPGQGLEWMGRISAGSGNVKYNEKFKGRVTITADTSTSTAYMELSSLRSEDTAVYYCAGSYYSYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 4019 - protein, 574 aa, synthetic construct
QVQLVQSGAEVKKPGSSVKVSCKASGHDFRLTYIHWVRQAPGQGLEWMGRVSAGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVADRVVWHQQKPGKAPKALIYSSSHRYKGVPSRFSGSGSGTEFTLTISSLQPEDFATYFCQQFKSYPLTFGQGTKLEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 4020 - protein, 368 aa, synthetic construct; identical to SEQ ID NO: 4002

SEQ ID NO: 4021 - protein, 574 aa, synthetic construct
QVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSSLSASVGDRVTITCKASQNVGINVVWHQQKPGKVPKALIYSSSHRYSGVPSRFSGSGSGTDFTLTISSLQPEDVATYFCQQFKSYPLTFGQGTKLEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 4022 - protein, 368 aa, synthetic construct; identical to SEQ ID NO: 4007

SEQ ID NO: 4023 - protein, 574 aa, synthetic construct
QVQLVQSGAEVKKPGSSVKVSCKASGTDFDKIYIHWVRQAPGQGLEWMGRVSAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 4024 - protein, 368 aa, synthetic construct; identical to SEQ ID NO: 4002

SEQ ID NO: 4025 - protein, 574 aa, synthetic construct
QVQLVQSGAEVKKPGSSVKVSCKASGHDFKLTYIHWVRQAPGQGLEWMGRVSPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWHQQKPGKAPKALIYSSSHRYKGVPSRFSGSGSGTEFTLTISSLQPEDFATYFCQQFKSYPLTFGQGTKLEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 4026 - protein, 368 aa, synthetic construct; identical to SEQ ID NO: 4015

SEQ ID NO: 4027 - protein, 574 aa, synthetic construct
QVQLVQSGAEVKKPGSSVKVSCKASGTDFDKTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 4028 - protein, 368 aa, synthetic construct; identical to SEQ ID NO: 4002

SEQ ID NO: 4029 - protein, 119 aa, synthetic construct; identical to SEQ ID NO: 188

SEQ ID NO: 4030 - protein, 370 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4031 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 4032 - protein, 6 aa, synthetic construct
HHHHHH

SEQ ID NO: 4033 - protein, 15 aa, synthetic construct
CASGDAWGSAETLYF

SEQ ID NO: 4034 - protein, 15 aa, synthetic construct
CASGDGWGGAETLYF

SEQ ID NO: 4035 - protein, 12 aa, synthetic construct
CASGDLGYEQYF

SEQ ID NO: 4036 - protein, 12 aa, synthetic construct
CASGTGAYEQYF

SEQ ID NO: 4037 - protein, 12 aa, synthetic construct
CASSRQNSDYTF

SEQ ID NO: 4038 - protein, 12 aa, synthetic construct
CASSAGTTEVFF

SEQ ID NO: 4039 - protein, 11 aa, synthetic construct
CASAGGYEQYF

SEQ ID NO: 4040 - protein, 13 aa, synthetic construct
CASRTGGSYEQYF

SEQ ID NO: 4041 - protein, 12 aa, synthetic construct
CASSAGGSDYTF

SEQ ID NO: 4042 - protein, 12 aa, synthetic construct
CASSDAGYEQYF

SEQ ID NO: 4043 - protein, 12 aa, synthetic construct
CASSDAQYEQYF

SEQ ID NO: 4044 - protein, 13 aa, synthetic construct
CASSDGGSYEQYF

SEQ ID NO: 4045 - protein, 12 aa, synthetic construct
CASSDGRYEQYF

SEQ ID NO: 4046 - protein, 12 aa, synthetic construct
CASSDGSYEQYF

SEQ ID NO: 4047 - protein, 13 aa, synthetic construct
CASSDGTGYEQYF

SEQ ID NO: 4048 - protein, 13 aa, synthetic construct
CASSDNSGNTLYF

SEQ ID NO: 4049 - protein, 12 aa, synthetic construct
CASSEGSYEQYF

SEQ ID NO: 4050 - protein, 13 aa, synthetic construct
CASSGDWGDEQYF

SEQ ID NO: 4051 - protein, 14 aa, synthetic construct
CASSLTGGAETLYF

SEQ ID NO: 4052 - protein, 12 aa, synthetic construct
CASSPGSYEQYF

SEQ ID NO: 4053 - protein, 15 aa, synthetic construct
CASGDAWGVAETLYF

SEQ ID NO: 4054 - protein, 11 aa, synthetic construct
CASSAFYEQYF

SEQ ID NO: 4055 - protein, 13 aa, synthetic construct
CASSDGWGYEQYF